(19) United States
(12) Patent Application Publication
(10) Pub. No.: US 2024/0360082 A1
(43) Pub. Date: Oct. 31, 2024

(54) SUBSTITUTED PYRIDONE COMPOUNDS USEFUL TO TREAT ORTHOMYXOVIRUS INFECTIONS

(71) Applicant: NOVARTIS AG, Basel (CH)

(72) Inventors: Graham Charles BLOOMFIELD, Essex (GB); Matthew James HESSE, Emeryville, CA (US); Richard Yichong HUANG, Emeryville, CA (US); Michael Robert MASER, Pasadena, CA (US); James Clifford SUTTON, Pleasanton, CA (US); Benjamin Robert TAFT, Emeryville, CA (US); David Charles TULLY, Danville, CA (US)

(73) Assignee: NOVARTIS AG, Basel (CH)

(21) Appl. No.: 18/291,083

(22) PCT Filed: Jul. 22, 2022
(86) PCT No.: PCT/IB2022/056778

§ 371 Date: Jan. 22, 2024

Related U.S. Application Data
(60) Provisional application No. 63/224,691, filed on Jul. 22, 2021.

(51) Int. Cl. C07D 213/82; A61K 31/4418; A61K 31/4427; A61K 31/443; A61K 31/4433; A61K 31/4436; A61K 31/4439; A61K 31/444; A61K 31/4545; A61K 31/4709; A61K 31/4725; A61K 31/497; A61K 31/498; A61K 31/501; A61K 31/506; A61K 31/5377; A61K 31/5383; A61K 31/5386; A61K 31/541; A61K 31/553; C07D 401/10; C07D 401/12; C07D 405/10; C07D 405/14; C07D 409/12; C07D 413/10; C07D 413/12; C07D 413/14; C07D 417/12; C07D 471/04; C07D 487/04; C07D 491/052; C07D 493/08; C07D 493/10; C07D 498/04; C07D 498/08; C07D 498/10

(52) U.S. Cl. CPC C07D 213/82; A61K 31/4418; A61K 31/4427; A61K 31/443; A61K 31/4433; A61K 31/4436; A61K 31/4439; A61K 31/444; A61K 31/4545; A61K 31/4709; A61K 31/4725; A61K 31/497; A61K 31/498; A61K 31/501; A61K 31/506; A61K 31/5377; A61K 31/5383; A61K 31/5386; A61K 31/541; A61K 31/553; C07D 401/10; C07D 401/12; C07D 405/10; C07D 405/14; C07D 409/12; C07D 413/10; C07D 413/12; C07D 413/14; C07D 417/12; C07D 471/04; C07D 487/04; C07D 491/052; C07D 493/08; C07D 493/10; C07D 498/04; C07D 498/08; C07D 498/10

(57) ABSTRACT

The disclosure provides compounds of Formula (I): (I) as further described herein, as well as pharmaceutical compositions comprising such compounds, and methods to use the compounds and pharmaceutical compositions for treatment of certain viral disorders, including influenza.

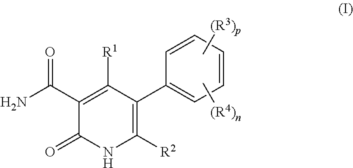

### CROSS-REFERENCE TO RELATED APPLICATIONS

This application claims the benefit of and priority to U.S. Provisional Patent Application Ser. No. 63/224,691, filed Jul. 22, 2021, the content of which is incorporated herein by reference in its entirety.

### FIELD

The disclosure provides compounds that inhibit orthomyxovirus replication, and are accordingly useful for treatment of viral infections caused by orthomyxoviruses. The disclosure further provides pharmaceutical compositions containing these compounds and methods of using these compounds to treat or prevent viral infections caused by orthomyxovirus.

### BACKGROUND

The orthomyxovirus family includes influenza A, influenza B and influenza C, all of which can infect humans, as well as several other genera of viruses that generally do not infect humans. Influenza infects millions of people and kills over 250,000 people globally each year. ‘The flu’ can vary in severity, but is usually only life-threatening for infants, the elderly, and those with underlying cardiopulmonary of immunologic disorders. However, the influenza virus is continually adapting, leading to emergence of new strains that are often more virulent, and these new strains have potential to cause pandemic outbreaks like the so-called Spanish flu (H1N1) that killed millions of people in 1918-1920, and was especially virulent in healthy young adults. Immunizations reduce the risk of infection, but they must be administered annually and are only effective against the particular strains that are predicted to be widespread in the upcoming flu season. Those predictions must be made months before flu season begins, and when the prediction of strain prevalence is wrong, immunizations provide limited protection.

The antivirals currently on the market for treating influenza target the M2 ion channel (amantadine and rimantadine), the neuroaminidase (e.g., oseltamivir), or the endonuclease (baloxavir). These must be administered at an early stage of infection in order to be effective, and resistance to both of these classes of antivirals has been documented (at varying rates depending upon the drug class). Thus there remains a need for new antivirals effective against influenza.

The influenza nucleoprotein (NP) has emerged as a new target for antiviral drug discovery. NP encapsidates viral RNA to form stable ribonucleoprotein (RNP) complexes. NP binds to the ribose-phosphate backbone of the viral RNA, leaving the RNA bases exposed to solvation and accessible to the viral polymerase, rendering the RNP complex is transcriptionally competent. While the precise mechanism by which they act is unclear, compounds that inhibit proper assembly of NP and/or RNP complexes have been shown to have antiviral activity. C. Cianci, et al., Antiviral Chemistry and Chemother., 23, 77-91 (2013).

Influenza A virus (IAV) is the most important of these pathogens in humans, often accounting for the great majority of serious cases of influenza during a typical flu season; importantly, all previous influenza pandemics have been caused by IAV. Thus, there is a special need for antiviral therapeutics effective to treat influenza A. The present disclosure provides new compounds that inhibit replication of orthomyxoviruses specifically influenza A virus (IAV), influenza B virus (IBV), and influenza C virus, and thereby may be useful in the treatment of influenza.

### BRIEF SUMMARY

Provided herein are compounds that inhibit replication of orthomyxoviruses, including influenza A, influenza B, and influenza C. Without being bound by theory, it is believed these compounds achieve their antiviral effects by inhibiting proper assembly of NP or RNP complexes.

In one aspect, the disclosure provide compounds of Formula (I), or stereoisomers thereof or pharmaceutically acceptable salts thereof:

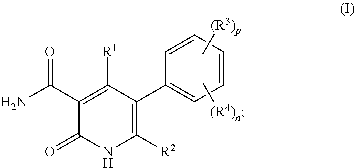

where R¹, R², R³ and R⁴ are as described herein.

Provided compounds include those of Formula (I), the subgenera of Formula (I) described herein, and all stereoisomers (including diastereoisomers and enantiomers), tautomers and isotopically enriched versions thereof (including deuterium substitutions), and pharmaceutically acceptable salts of these compounds.

The compounds of the disclosure are inhibitors of the function of influenza nucleoprotein as shown by the data provided herein, and they inhibit replication of influenza viruses. Accordingly, these compounds are useful to treat or prevent orthomyxovirus infections in mammals susceptible to such infections, and are particularly useful to treat influenza virus infections in humans. They are also useful to inhibit replication of orthomyxoviruses, including influenza viruses, in cells.

In another aspect, the disclosure provides pharmaceutical compositions comprising a provided compound and at least one pharmaceutically acceptable carrier or excipient. In certain embodiments such pharmaceutical compositions comprise a compound as disclosed herein and two or more pharmaceutically acceptable carriers or excipients. In an embodiment of this aspect such pharmaceutical compositions further comprise a therapeutically effective amount of at least one other antiviral agent. In an embodiment of this aspect such pharmaceutical compositions further comprise a therapeutically effective amount of one or more therapeutically active co-agents.

In another aspect, the disclosure provides a method to treat a subject infected with influenza A, B, or C, where the method comprises administering to a subject in need of such treatment an effective amount of a provided compound.

In another aspect, the disclosure provides a method to treat a subject infected with influenza A, B, or C, where the method comprises administering to a subject in need of such treatment an effective amount of a provided compound alone or in combination with at least one other antiviral agent, administered together or separately.

Another aspect of the disclosure is a method of inhibiting influenza virus nucleoprotein (NP), where the method comprises contacting NP with a provided compound.

Another aspect of the disclosure is a method of preventing or treating influenza, where the method comprises administering to a subject a therapeutically effective amount of a provided compound.

Another aspect of the disclosure is a method of treating influenza, where the method comprises administering to a subject in need thereof a therapeutically effective amount of a provided compound.

Another aspect of the disclosure is the use of a provided compound n the manufacture of a medicament for the treatment or prevention of influenza.

Another aspect of the disclosure is the use of a provided compound in the manufacture of a medicament for the treatment or prevention of influenza A, B, or C.

Another aspect of the disclosure is the use of a provided compound as a medicament for the treatment or prevention of influenza.

Another aspect of the disclosure is the use of a provided compound as a medicament for the treatment or prevention of influenza A, B, or C.

Another aspect of the disclosure is the use of a provided compound for the treatment or prevention of influenza.

Another aspect of the disclosure is the use of a provided compound for the treatment or prevention of influenza A, B, or C.

### DETAILED DESCRIPTION

### Definitions

For purposes of interpreting this specification, the following definitions will apply, and whenever appropriate, terms used in the singular will also include the plural. Terms used in the specification have the following meanings unless the context clearly indicates otherwise.

The term “alkyl,” as used herein, refers to a fully saturated branched or straight chain hydrocarbon. In certain embodiments an alkyl group is a “C₁-C₂alkyl”, “C₁-C₃alkyl”, “C₁-C₄alkyl”, “C₁-C₅alkyl”, “C₁-C₆alkyl”, “C₁-C₇alkyl”, “C₁-C₈alkyl”, “C₁-C₉alkyl” or “C₁-C₁₀alkyl”, wherein the terms “C₁-C₂alkyl”, “C₁-C₃alkyl”, “C₁-C₄alkyl”, “C₁-C₅alkyl”, “C₁-C₆alkyl”, “C₁-C₇alkyl”, “C₁-C₈alkyl”, “C₁-C₉alkyl” or “C₁-C₁₀alkyl”, as used herein, refer to an alkyl group containing at least 1, and at most 2, 3, 4, 5, 6, 7, 8, 9 or 10 carbon atoms, respectively. Non-limiting examples of alkyl groups include methyl, ethyl, n-propyl, isopropyl, n-butyl, isobutyl, sec-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene,” as used herein, refers to a saturated branched or straight chain divalent hydrocarbon radical derived from an alkyl group as defined herein. In certain embodiments an alkylene group is a “C₁-C₃alkylene”, “C₁-C₄alkylene”, “C₁-C₅alkylene”, “C₁-C₆alkylene”, “C₁-C₇alkylene”, “C₁-C₈alkylene”, “C₁-C₉alkylene” or “C₁-C₁₀alkylene”, wherein the terms “C₁-C₃alkylene”, “C₁-C₄alkylene”, “C₁-C₅alkylene”, “C₁-C₆alkylene”, “C₁-C₇alkylene” and “C₁-C₈alkylene”, as used herein, refer to an alkylene group containing at least 1, and at most 3, 4, 5, 6, 7, 8, 9 or 10 carbon atoms respectively. Non-limiting examples of alkylene groups as used herein include, methylene, ethylene, n-propylene, isopropylene, n-butylene, isobutylene, sec-butylene, t-butylene, n-pentylene, isopentylene, hexylene, heptylene, octylene, nonylene, decylene and the like.

The term “alkoxy”, as used herein, refers to —O-alkyl or -alkyl-O—, wherein “alkyl” is as defined herein. In certain embodiments an alkoxy group is a “C₁-C₂alkoxy”, “C₁-C₃alkoxy”, “C₁-C₄alkoxy”, “C₁-C₅alkoxy”, “C₁-C₆alkoxy”, “C₁-C₇alkoxy”, “C₁-C₈alkoxy”, “C₁-C₉alkoxy” or “C₁-C₁₀alkoxy”, wherein the terms “C₁-C₃alkoxy”, “C₁-C₄alkoxy”, “C₁-C₅alkoxy”, “C₁-C₆alkoxy”, “C₁-C₇alkoxy”, “C₁-C₈alkoxy”, “C₁-C₇alkoxy” and “C₁-C₁₀alkoxy”, as used herein refer to —O—C₁-C₂alkyl, —O—C₁-C₃alkyl, —O—C₁-C₄alkyl, —O—C₁-C₅alkyl, —O—C₁-C₆alkyl, —O—C₁-C₇alkyl, —O—C₁-C₈alkyl, —O—C₁-C₉alkyl or —O—C₁-C₁₀alkyl, respectively. Non-limiting examples of “alkoxy” groups include methoxy, ethoxy, n-propoxy, isopropoxy, n-butoxy, isobutoxy, sec-butoxy, tert-butoxy, n-pentoxy, isopentoxy, hexoxy, heptoxy, octoxy, nonoxy and decoxy.

The term “C₃-C₈cycloalkyl” as used herein, refers to a fully saturated, monocyclic hydrocarbon ring system having 3 to 8 carbon atoms as ring members. Non-limiting examples of such “C₃-C₈cycloalkyl” groups include cyclopropyl, cyclobutyl, cyclopentyl, cyclohexyl, cycloheptyl and cyclooctyl groups.

The terms “halo” or “halogen” as used herein, refer to fluoro (F), chloro (Cl), bromo (Br) or iodo (I).

The term “haloalkyl” as used herein, refers to an alkyl group as defined herein, wherein at least one of the hydrogen atoms of the alkyl is replaced by a halo group (as defined herein). The haloalkyl may be monohaloalkyl, dihaloalkyl, trihaloalkyl, or polyhaloalkyl including perhaloalkyl. A monohaloalkyl may have one iodo, bromo, chloro or fluoro within the alkyl group. Dihaloalkyl and polyhaloalkyl groups may have two or more of the same halo atoms or a combination of different halo groups within the alkyl. Typically the polyhaloalkyl contains up to 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-alkyl refers to an alkyl having all hydrogen atoms replaced with halo atoms, e.g., trifluoromethyl. Preferred haloalkyl groups, unless specified otherwise, include monofluoro-, difluoro- and trifluoro-substituted methyl and ethyl groups, e.g. CF₃, CHF₂, CH₂F, CH₂CHF₂ and CH₂CF₃.

The term “C₁-C₈haloalkyl” as used herein, refers to the respective “C₁-C₈alkyl”, as defined herein, wherein at least one of the hydrogen atoms of the “C₁-C₈alkyl” is replaced by a halo group (as defined herein). The C₁-C₈haloalkyl groups may be monoC₁-C₈haloalkyl, wherein such C₁-C₈haloalkyl groups have one iodo, one bromo, one chloro or one fluoro. Additionally, the C₁-C₈haloalkyl groups may be diC₁-C₈haloalkyl wherein such C₁-C₈haloalkyl groups may have two halo atoms independently selected from iodo, bromo, chloro or fluoro. Furthermore, the C₁-C₈haloalkyl groups may be polyC₁-C₈haloalkyl wherein such C₁-C₈haloalkyl groups may have two or more of the same halo atoms or a combination of two or more different halo atoms. Such polyC₁-C₈haloalkyl may be perhaloC₁-C₈haloalkyl where all the hydrogen atoms of the respective C₁-C₈alkyl have been replaced with halo atoms and the halo atoms may be the same or a combination of different halo atoms. Non-limiting examples of “C₁-C₈haloalkyl” groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, fluoroethyl, difluoroethyl, trifluoroethyl, difluoropropyl, dichloroethyl and dichloropropyl.

The term “haloalkoxy” as used herein, refers to the group —O-haloalkyl wherein at least one of the hydrogen atoms of the alkyl group of the alkoxy is replaced by a halo group (as defined herein). The haloalkoxy may be monohaloalkoxy, dihaloalkoxy, trihaloalkoxy, or polyhaloalkoxy including perhaloalkoxy. A monohaloalkoxy may have one iodo, bromo, chloro or fluoro within the alkyl group. Dihaloalkoxy and polyhaloalkoxy groups may have two or more of the same halo atoms or a combination of different halo groups within the alkyl. Typically, the polyhaloalkoxy contains up to 6, or 4, or 3, or 2 halo groups. Non-limiting examples of haloalkoxy include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, difluoroethoxy, difluoropropoxy, dichloroethoxy and dichloropropoxy. A perhalo-alkoxy refers to an alkoxy having all hydrogen atoms replaced with halo atoms, e.g., trifluoromethoxy. Preferred haloalkoxy groups, unless specified otherwise, include monofluoro-, difluoro- and trifluoro-substituted methoxy and ethoxygroups, e.g. —OCF₃, —OCHF₂, —OCH₂F, —OCH₂CHF₂ and —OCH₂CF₃.

The term “C₁-C₈haloalkoxy” as used herein, refers to the group —O—C₁-C₈haloalkyl, wherein at least one of the hydrogen atoms of the “C₁-C₈alky” of the “C₁-C₈alkoxy” is replaced by a halo group (as defined herein). The C₁-C₈haloalkoxy groups may be monoC₁-C₈haloalkoxy, wherein such C₁-C₈haloalkoxy groups have one iodo, one bromo, one chloro or one fluoro. Additionally, the C₁-C₈haloalkoxy groups may be diC₁-C₈haloalkoxy wherein such C₁-C₈haloalkoxy groups may have two halo atoms independently selected from iodo, bromo, chloro or fluoro. Furthermore, the C₁-C₈haloalkoxy groups may be polyC₁-C₈haloalkoxy wherein such C₁-C₈haloalkoxy groups may have two or more of the same halo atoms or a combination of two or more different halo atoms. Such polyC₁-C₈haloalkoxy may be perhaloC₁-C₈haloalkoxy where all the hydrogen atoms of the respective C₁-C₈alkoxy have been replaced with halo atoms and the halo atoms may be the same or a combination of different halo atoms. Non-limiting examples of “C₁-C₈haloalkoxy” groups include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, fluoroethoxy, difluoroethoxy, trifluoroethoxy, difluoropropoxy, dichloroethoxy and dichloropropoxy.

The term “5-6 membered heteroaryl”, as used herein, refers to an aromatic, 5 membered or 6 membered system having 1 to 3 ring members independently selected from N, NR⁷, O and S, where R⁷ is as defined herein. Non-limiting examples of such 5 membered heteroaryl groups, as used herein, include furyl, imidazolyl, isoxazolyl, isothiazolyl, oxazolyl, pyrrolyl, pyrazolyl, thiadiazolyl, thiazolyl, thienyl, triazolyl, pyridyl, pyridazinyl, pyrazinyl, and pyrimidinyl.

The term “9-10 membered heteroaryl”, as used herein, refers to an aromatic, 9 or 10 membered fused bicyclic ring system having 1 to 4 ring members independently selected from N, NR⁷, N⁺O⁻, O and S, where R⁷ is as defined herein. Non-limiting examples of such bicyclic heteroaryl groups, as used herein, include indolyl, quinolinyl, isoquinolinyl, indazolyl, purinyl, phthalazinyl, naphthyridinyl, quinazolinyl, cinnolinyl, quinoxalinyl, benzo[c][1,2,5]oxadiazolyl, tetrazolo[1,5-a]pyridinyl, imidazo[1,2-a]pyridinyl, imidazo[4,5-b]pyridinyl, triazolo[4,3-a]pyrimidinyl, thieno[2,3-b]furanyl, 1H-pyrazolo[4,3-d]-oxazolyl, imidazo[2,1-b] thiazolyl, pyrazino[2,3-d]pyridazinyl, imidazo[1,2-b][1,2,4]triazinyl, benzoxazolyl, benzimidazolyl, imidazopyridinyl and benzothiazolyl. In certain embodiments such a bicyclic heteroaryl group is 1H-benzo[d]imidazolyl or 1H-imidazo[4,5-c]pyridinyl.

The term “heteroatoms” or“hetero atoms”, as used herein, refers to nitrogen (N), oxygen (O) or sulfur (S) atoms.

The term “4-8 monocyclic heterocycloalkyl,” as used herein refers to a cycloalkyl group as defined herein having one to three carbon atoms in the ring structure being replaced with one to three groups independently selected from N, NH, NR⁷, O, S, C═O and S(═O)₂, wherein R⁷ is as defined herein. In particular the 4-8 monocyclic heterocycloalkyl is a fully saturated, monocyclic hydrocarbon ring structure having 4 to 8 ring members, wherein one to three of the ring members are independently selected from N, NH, NR⁷, O, S, C═O and S(═O)₂, wherein R⁷ is as defined herein. Non-limiting examples of heterocycloalkyl groups, as used herein, include oxazepanyl, oxomorpholinyl, dioxidothiomorpholinyl, dioxanyl, dioxepanyl, oxopyrrolidinyl, oxooxazolidinyl, azetidinyl, azetidin-1-yl, azetidin-2-yl, azetidin-3-yl, oxetanyl, oxetan-2-yl, oxetan-3-yl, oxetan-4-yl, thietanyl, thietan-2-yl, thietan-3-yl, thietan-4-yl, pyrrolidinyl, pyrrolidin-1-yl, pyrrolidin-2-yl, pyrrolidin-3-yl, pyrrolidin-4-yl, pyrrolidin-5-yl, tetrahydrofuranyl, tetrahydrofuran-2-yl, tetrahydrofuran-3-yl, tetrahydrofuran-4-yl, tetrahydrofuran-5-yl, tetrahydrothienyl, tetrahydrothien-2-yl, tetrahydrothien-3-yl, tetrahydrothien-4-yl, tetrahydrothien-5-yl, piperidinyl, piperidin-1-yl, piperidin-2-yl, piperidin-3-yl, piperidin-4-yl, piperidin-5-yl, piperidin-6-yl, tetrahydropyranyl, tetrahydropyran-2-yl, tetrahydropyran-3-yl, tetrahydropyran-4-yl, tetrahydropyran-5-yl, tetrahydropyran-6-yl, tetrahydrothiopyranyl, tetrahydrothiopyran-2-yl, tetrahydrothiopyran-3-yl, tetrahydrothiopyran-4-yl, tetrahydrothiopyran-5-yl, tetrahydrothiopyran-6-yl, piperazinyl, piperazin-1-yl, piperazin-2-yl, piperazin-3-yl, piperazin-4-yl, piperazin-5-yl, piperazin-6-yl, morpholinyl, morpholin-2-yl, morpholin-3-yl, morpholin-4-yl, morpholin-5-yl, morpholin-6-yl, thiomorpholinyl, thiomorpholin-2-yl, thiomorpholin-3-yl, thiomorpholin-4-yl, thiomorpholin-5-yl, thiomorpholin-6-yl, oxathianyl, oxathian-2-yl, oxathian-3-yl, oxathian-5-yl, oxathian-6-yl, dithianyl, dithian-2-yl, dithian-3-yl, dithian-5-yl, dithian-6-yl, dioxolanyl, dioxolan-2-yl, dioxolan-4-yl, dioxolan-5-yl, thioxanyl, thioxan-2-yl, thioxan-3-yl, thioxan-4-yl, thioxan-5-yl, dithiolanyl, dithiolan-2-yl, dithiolan-4-yl, dithiolan-5-yl, pyrazolidinyl, pyrazolidin-1-yl, pyrazolidin-2-yl, pyrazolidin-3-yl, pyrazolidin-4-yl, pyrazolidin-5-yl, 2-azabicyclo[4.2.0]octanyl, octahydro-1H-cyclopenta[b]pyridine and decahydroquinoline.

The term “9-12 membered heterocyclyl group” as used herein refers to a 9 to 12 membered, partially saturated hydrocarbon ring structure having 1 to 4 ring members independently selected from N, NH, NR⁷, O or —S—, wherein R⁷ is as defined herein, wherein the partially saturated hydrocarbon ring structure may be a monocyclic, a fused bicyclic or a fused tricyclic ring system. The heterocyclic group may be attached to another group at a nitrogen or a carbon atom. In certain embodiments a heterocyclyl can be a 9 to 12 membered fused bicyclic heterocyclyl ring structure having 1 to 4 ring members independently selected from N, NR⁷, O and S, where R⁷ is as defined herein. Non-limiting examples of heterocyclyl groups, as used herein, include benzo[d][1,3]dioxolyl, benzo[b][1,4]dioxepinyl, tetrahydrobenzo[d]isoxazolyl, dihydrobenzofuranyl, tetrahydropyrano[3,4-c]pyrazolyl, chromanyl and 6,7-dihydro-4H-[1,2,3]triazolo[5,1-c][1,4]oxazinyl.

As used herein, the term “an optical isomer” or “a stereoisomer” refers to any of the various stereo isomeric configurations which may exist for a given compound of the present disclosure and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner.

Therefore, the disclosure includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog ‘R—S’ system. When a compound is a pure enantiomer, the stereochemistry at each chiral carbon may be specified by either R or S. Resolved compounds whose absolute configuration is unknown may be designated (+) or (−) depending on the direction (dextro- or levorotatory) which they rotate plane polarized light at the wavelength of the sodium D line.

Certain compounds described herein contain one or more asymmetric centers or axes and may thus give rise to enantiomers, diastereomers, and other stereoisomeric forms that may be defined, in terms of absolute stereochemistry, as (R)- or (S)-.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art for use in a pharmaceutical composition for administration to a human subject (see, for example, Remington: The Science and Practice of Pharmacy, 22nd ed.). Except insofar as any conventional carrier is incompatible with the active ingredient, its use in the therapeutic or pharmaceutical compositions is contemplated.

As used herein, the term “a therapeutically effective amount” of a compound refers to an amount of the compound of the present disclosure that will elicit the biological or medical response in a subject, for example, an amount sufficient to reduce of one or more symptoms, alleviate conditions, slow or delay disease progression, or prevent a disease, etc.

In one non-limiting embodiment, the term “a therapeutically effective amount” refers to the amount of a compound of the present disclosure that, when administered to a subject, is effective to reduce one or more symptoms associated with an influenza virus infection, or to shorten the duration of the symptomatic stage of an influenza virus infection, or to slow the progression of an influenza virus infection, or to reduce or stop the exacerbation of an underlying condition by an influenza virus infection.

In another non-limiting embodiment, the term “a therapeutically effective amount” refers to the amount of the compound of the present disclosure that, when administered to a cell, or a tissue, or a non-cellular biological material, or a medium, is effective to cause a statistically significant reduction in rate of replication or proliferation of a strain of orthomyxovirus.

As used herein, the term “subject” refers to an animal. Typically, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof).

In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discemible symptom), physiologically, (e.g., stabilization of a physical parameter), or both. In yet another embodiment, “treat”, “treating” or “treatment” refers to preventing or delaying the development or progression of the disease or disorder.

As used herein, a subject is “in need of” a treatment if such subject would benefit biologically, medically or in quality of life from such treatment.

As used herein, the terms “a,” “an,” “the” and similar terms used in the context of the present disclosure (especially in the context of the claims) are to be construed to cover both the singular and plural unless otherwise indicated herein or clearly contradicted by the context.

The compound names provided herein were obtained using ChemDraw Professional version 17.1 (PerkinElmer Inc.).

Unless specified otherwise, the term “compounds of the present disclosure”, “compounds of the disclosure” or “compounds provided herein” refers to compounds of Formula (I), and sub-formulae thereof, including Formula (II), Formula (III), Formula (IV), Formula (V), Formula (V-a), Formula (V-b), Formula (V-c), Formula (V-a1), Formula (V-a2), Formula (V-b1), Formula (V-b2), Formula (V-b3), Formula (V-b4), Formula (V-c1), Formula (V-c2), Formula (V-c3), Formula (V-c4), Formula (V-d1), Formula (V-d2), Formula (V-d3), Formula (V-d4), Formula (V-e1), Formula (V-e2), Formula (V-e3) and Formula (V-e4), and pharmaceutically acceptable salts, stereoisomers (including diastereoisomers and enantiomers), rotamers, tautomers and isotopically labeled compounds (including deuterium substitutions), as well as inherently formed moieties.

### Compounds

The disclosure provides compounds having the structure of Formula (I), or a stereoisomer thereof or pharmaceutically acceptable salt thereof:

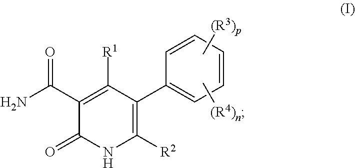

wherein:
R¹ is H or halo;
R² is —C₁-C₈haloalkyl;
R³ is L₁R⁵ or L₂R;
R⁴ is halo, CN, C₁-C₈alkyl, C₁-C₈alkoxy or C₃-C₈cycloalkyl;
L₁ is a bond, —CH₂—, —(CH₂)ₘ—, —OCH₂—, —O—, —CH₂O—, —O(CH₂)ₘ—, —CH₂OCH₂—, —CH(R⁷)—, —OCH(R⁷)—, —CH(R¹⁰)—, —OCH(R¹⁰)—, —CF₂—, —CF₂CH₂—, —OCF₂—, —OCH₂C(═O)—, —OCH₂C(═O)NH—, —OCH₂CH(OH)CH₂—, —CH₂OCH₂C(R⁷)₂—, —C(R⁷)—, —OC(R⁷)₂—, —OCH(R¹⁰)CH₂—, —NH— or —NH(CH₂)ₘ—;
L₂ is a bond, —CH₂—, —(CH₂)ₘ—, —OCH₂—, —CH₂O—, —O(CH₂)ₘ—, —CH₂OCH₂—, —CH(R⁷)—, —OCH(R⁷)—, —CH(R¹⁰)—, —OCH(R¹⁰)—, —C(R⁷)₂—, —OC(R⁷)₂—, —CF₂—, —CF₂CH₂—, —OCF₂—, —OCH₂C(═O)NH—, —OCH₂CH(OH)CH₂—, —CH₂OCH₂C(R⁷)₂—, —NH(CH₂)ₘ—, —OCH(R¹⁰)CH₂— or —OCH₂C(═O)—;
L₃ is a bond, —CH₂—, —(CH₂)ₘ—, —OCH₂—, —O—, —NH—, —NH(CH₂)ₘ—, —OCH₂C(═O)—, —OCH₂C(═O)NH—, —OCH₂CH(OH)CH₂—, —CH₂OCH₂—, —CH₂O—, —O(CH₂)ₘ—, —CH(R⁷)—, —OCH(R⁷)—, —CH(R¹⁰)—, —OCH(R¹⁰)—, —CH₂OCH₂C(R⁷)₂—, —CF₂—, —CF₂CH₂—, —OCF₂—, —C(R⁷)₂—, —OC(R⁷)₂—, or —OCH(R¹⁰)CH₂—;
L₄ is a bond, —CH₂—, —(CH₂)ₘ—, —C(R⁷)₂—, —OC(R⁷)₂, —CF₂—, —OCH₂—, —O(CH₂)ₘ—, —CH₂O—, —CH₂OCH₂—, —CH₂OCH₂C(R⁷)₂—, —NH(CH₂)ₘ—, —OCH₂C(═O)—, —OCH₂C(═O)NH—, —OCH₂CH(OH)CH₂—, —CF₂CH₂—, —OCF₂—, —CH(R¹⁰)—, —OCH(R¹⁰)—, —OCH(R⁷)—, —OC(R⁷)₂—, —CH(R⁷)—, or —OCH(R¹⁰)CH₂;
R⁵ is selected from the group consisting of
   i) phenyl substituted with 0-3 groups independently selected from R⁹;
   ii) C₃-C₈cycloalkyl substituted with 0-3 groups independently selected from R⁹;
   iii) a 4-8 membered monocyclic heterocycloalkyl group having 1 to 3 ring members independently selected from N, NR⁷, O, S, C═O or S(═O)₂, substituted with 0-3 groups independently selected from R⁹;
   iv) a 9-12 membered heterocyclyl group having 1 to 4 ring members independently selected from N, NR⁷, O, or S, substituted with 0-3 groups independently selected from R⁹;
   v) a 7-8 membered bridged-bicyclic heterocycloalkyl group having 1 to 2 ring members independently selected from N, NR⁷, O, S, C═O or S(═O)₂, substituted with 0-3 groups independently selected from R⁹;
   vi) a 6-8 membered fused bicyclic heterocycloalkyl group having 1 to 2 ring members independently selected from N, NR⁷, O, S, C═O or S(═O)₂, substituted with 0-3 groups independently selected from R⁹;
   vii) a 5-6 membered heteroaryl having 1 to 3 ring members independently selected from N, NR⁷, O or S, substituted with 0-3 groups independently selected from R⁹;
   and
   viii) a 9-10 membered heteroaryl having 1 to 4 ring members independently selected from N, NR⁷, N⁺O⁻, O, or S, substituted with 0-3 groups independently selected from R⁹;
R⁶ is —CN, —OH, —NR⁷R⁸, —NR⁷C(═O)R⁸, —NR⁷S(═O)₂R⁸, —C(═O)NR⁷R⁸, —S(═O)₂R⁸, halo, C₁-C₈haloalkoxy, C₁-C₈-alkoxy, —C₁-C₈alkyl or —C₁-C₈alkylene;
each R⁷ is independently selected from H or —C₁-C₈alkyl;
R⁸ is H or —C₁-C₈alkyl;
each R⁹ is independently selected from —C₁-C₈alkyl, —C₁-C₈-alkoxy, C₁-C₈haloalkyl, spiro attached C₃-C₈cycloalkyl, R¹⁰, —OH, CN, halo, —C(═O)R⁷, —NR⁷R⁸, —C(═O)NR⁷R⁸, —NR⁷C(═O)R⁸, —NR⁷C(═O)OR⁸, —C(═O)OR⁸, —NR⁷S(═O)₂R⁸, —S(═O)₂R⁸, —S(═O)₂R¹⁰, NO₂, CN, L₃R¹¹, L₄R¹² or a spiro attached 4-8 membered monocyclic heterocycloalkyl group having 1 to 2 ring members independently selected from N, NR⁷, O, or S;
R¹⁰ is a C₃-C₈cycloalkyl, a 4-8 membered monocyclic heterocycloalkyl group having 1 to 2 ring members independently selected from N, NR⁷, O or S, or a 5-6 membered heteroaryl having 1 to 2 ring members independently selected from N, NR⁷, O, or S;
each R¹¹ is independently selected from the group consisting of
   i) phenyl substituted with 0-3 groups independently selected from R¹³;
   ii) C₃-C₈cycloalkyl substituted with 0-3 groups independently selected from R¹³;
   iii) a 4-8 membered monocyclic heterocycloalkyl group having 1 to 3 ring members independently selected from N, NR⁷, O, S, C═O or S(═O)₂ substituted with 0-3 groups independently selected from R¹³;
   iv) a 9-12 membered heterocyclyl having 1 to 4 ring members independently selected from N, NR⁷, O, or S substituted with 0-3 groups independently selected from R¹³;
   v) a 7-8 membered bridged-bicyclic heterocycloalkyl group having 1 to 2 ring members independently selected from N, NR⁷, O, S, C═O or S(═O)₂, substituted with 0-3 groups independently selected from R¹³;
   vi) a 6-8 membered fused bicyclic heterocycloalkyl group having 1 to 2 ring members independently selected from N, NR⁷, O, S, C═O or S(═O)₂, substituted with 0-3 groups independently selected from R¹³;
   vii) a 5-6 membered heteroaryl having 1 to 3 ring members independently selected from N, NR⁷, O or S substituted with 0-3 groups independently selected from R¹³,
   and
   viii) a 9-10 membered heteroaryl having 1 to 4 ring members independently selected from N, NR⁷, N⁺O⁻, O, or S substituted with 0-3 groups independently selected from R¹³;
each R¹² is independently selected from —CN, —CD₃, —OCD₃, —OH, —NR⁷R⁸, —NR⁷C(═O)R⁸, —NR⁷S(═O)₂R⁸, —C(═O)NR⁷R⁸, —S(═O)₂R⁸, —S(═O)₂R¹⁰, halo, C₁-C₈haloalkoxy, C₁-C₈-alkoxy, C₁-C₈haloalkyl or —C₁-C₈alkyl;
each R¹³ is independently selected from —C₁-C₈alkyl, spiro attached C₃-C₈cycloalkyl, —C₁-C₈-alkoxy, —OH, halo, —C(═O)R⁷, —NR⁷R⁸, —C(═O)NR⁷R⁸, —NR⁷C(═O)R⁸, —NR⁷S(═O)₂R⁸, —S(═O)₂R⁸, NO₂, CN or C₁-C₈haloalkyl;
each m is independently selected from 1, 2, 3 or 4;
each n is independently selected from 0, 1, 2 or 3; and
each p is independently selected from 0, 1, 2 or 3.

Various embodiments are described herein. It will be recognized that features specified in each embodiment may be combined with other specified features to provide further embodiments. The following enumerated embodiments are representative of aspects of the disclosure.

Embodiment 1. A compound having the structure of Formula (I), or a stereoisomer thereof or pharmaceutically acceptable salt thereof

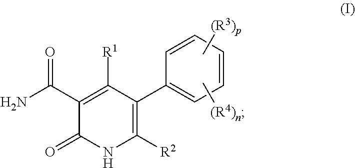

wherein:
R¹ is H or halo;
R² is —C₁-C₈haloalkyl;
R³ is L₁R⁵ or L₂R;
R⁴ is halo, CN, C₁-C₈alkyl, C₁-C₈alkoxy or C₃-C₈cycloalkyl;
L₁ is a bond, —CH₂—, —(CH₂)ₘ—, —OCH₂—, —O—, —CH₂O—, —O(CH₂)ₘ—, —CH₂OCH₂, —CH(R⁷)—, —OCH(R⁷)—, —CH(R¹⁰)—, —OCH(R¹⁰)—, —CF₂—, —CF₂CH₂—, —OCF₂—, —OCH₂C(═O)—, —OCH₂C(═O)NH—, —OCH₂CH(OH)CH₂—, —CH₂OCH₂C(R⁷)₂—, —C(R⁷)—, —OC(R⁷)₂—, —OCH(R¹⁰)CH₂—, —NH— or —NH(CH₂)ₘ—;
L₂ is a bond, —CH₂—, —(CH₂)ₘ—, —OCH₂—, —CH₂O—, —O(CH₂)ₘ—, —CH₂OCH₂—, —CH(R⁷)—, —OCH(R⁷)—, —CH(R¹⁰)—, —OCH(R¹⁰)—, —C(R⁷)₂—, —OC(R⁷)₂—, —CF₂—, —CF₂CH₂—, —OCF₂—, —OCH₂C(═O)NH—, —OCH₂CH(OH)CH₂—, —CH₂OCH₂C(R⁷)₂—, —NH(CH₂)ₘ—, —OCH(R¹⁰)CH₂— or —OCH₂C(═O)—;
L₃ is a bond, —CH₂—, —(CH₂)ₘ—, —OCH₂—, —O—, —NH—, —NH(CH₂)ₘ—, —OCH₂C(═O)—, —OCH₂C(═O)NH—, —OCH₂CH(OH)CH₂—, —CH₂OCH₂—, —CH₂O—, —O(CH₂)ₘ—, —CH(R⁷)—, —OCH(R⁷)—, —CH(R¹⁰)—, —OCH(R¹⁰)—, —CH₂OCH₂C(R⁷)₂—, —CF₂—, —CF₂CH₂—, —OCF₂—, —C(R⁷)₂—, —OC(R⁷)₂—, or —OCH(R¹⁰)CH₂—;
L₄ is a bond, —CH₂—, —(CH₂)ₘ—, —C(R⁷)₂—, —OC(R⁷)₂, —CF₂—, —OCH₂—, —O(CH₂)ₘ—, —CH₂O—, —CH₂OCH₂—, —CH₂OCH₂C(R⁷)₂—, —NH(CH₂)ₘ—, —OCH₂C(═O)—, —OCH₂C(═O)NH—, —OCH₂CH(OH)CH₂—, —CF₂CH₂—, —OCF₂—, —CH(R¹⁰)—, —OCH(R¹⁰)—, —OCH(R⁷)—, —OC(R⁷)₂—, —CH(R⁷)—, or —OCH(R¹⁰)CH₂;
R⁵ is selected from the group consisting of
   i) phenyl substituted with 0-3 groups independently selected from R⁹;
   ii) C₃-C₈cycloalkyl substituted with 0-3 groups independently selected from R⁹;
   iii) a 4-8 membered monocyclic heterocycloalkyl group having 1 to 3 ring members independently selected from N, NR⁷, O, S, C═O or S(═O)₂, substituted with 0-3 groups independently selected from R⁹;
   iv) a 9-12 membered heterocyclyl group having 1 to 4 ring members independently selected from N, NR⁷, O, or S, substituted with 0-3 groups independently selected from R⁹;
   v) a 7-8 membered bridged-bicyclic heterocycloalkyl group having 1 to 2 ring members independently selected from N, NR⁹, O, S, C═O or S(═O)₂, substituted with 0-3 groups independently selected from R⁹;
   vi) a 6-8 membered fused bicyclic heterocycloalkyl group having 1 to 2 ring members independently selected from N, NR⁹, O, S, C═O or S(═O)₂, substituted with 0-3 groups independently selected from R⁹;
   vii) a 5-6 membered heteroaryl having 1 to 3 ring members independently selected from N, NR⁷, O or S, substituted with 0-3 groups independently selected from R⁹;
   and
   viii) a 9-10 membered heteroaryl having 1 to 4 ring members independently selected from N, NR⁷, N⁺O⁻, O, or S, substituted with 0-3 groups independently selected from R⁹;
R⁶ is —CN, —OH, —NR⁷R⁸, —NR⁷C(═O)R⁸, —NR⁷S(═O)₂R⁸, —C(═O)NR⁷R⁸, —S(═O)₂R⁹, halo, C₁-C₈haloalkoxy, C₁-C₈-alkoxy, —C₁-C₈alkyl or —C₁-C₈alkylene;
each R⁷ is independently selected from H or —C₁-C₈alkyl;
R⁸ is H or —C₁-C₈alkyl;
each R⁹ is independently selected from —C₁-C₈alkyl, —C₁-C₈-alkoxy, C₁-C₈haloalkyl, spiro attached C₃-C₈cycloalkyl, R¹⁰, —OH, CN, halo, —C(═O)R⁷, —NR⁷R⁸, —C(═O)NR⁷R⁸, —NR⁷C(═O)R⁸, —NR⁷C(═O)OR⁸, —C(═O)OR⁸, —NR⁷S(═O)₂R⁸, —S(═O)₂R⁸, —S(═O)₂R¹⁰, NO₂, CN, L₃R¹¹, L₄R¹² or a spiro attached 4-8 membered monocyclic heterocycloalkyl group having 1 to 2 ring members independently selected from N, NR⁷, O, or S;
R¹⁰ is a C₃-C₈cycloalkyl, a 4-8 membered monocyclic heterocycloalkyl group having 1 to 2 ring members independently selected from N, NR⁷, O or S, or a 5-6 membered heteroaryl having 1 to 2 ring members independently selected from N, NR⁷, O, or S;
each R¹¹ is independently selected from the group consisting of
   i) phenyl substituted with 0-3 groups independently selected from R¹³;
   ii) C₃-C₈cycloalkyl substituted with 0-3 groups independently selected from R¹³;
   iii) a 4-8 membered monocyclic heterocycloalkyl group having 1 to 3 ring members independently selected from N, NR⁷, O, S, C═O or S(═O)₂ substituted with 0-3 groups independently selected from R¹³;
   iv) a 9-12 membered heterocyclyl having 1 to 4 ring members independently selected from N, NR⁷, O, or S substituted with 0-3 groups independently selected from R¹³;
   v) a 7-8 membered bridged-bicyclic heterocycloalkyl group having 1 to 2 ring members independently selected from N, NR⁷, O, S, C═O or S(═O)₂, substituted with 0-3 groups independently selected from R¹³;
   vi) a 6-8 membered fused bicyclic heterocycloalkyl group having 1 to 2 ring members independently selected from N, NR⁷, O, S, C═O or S(═O)₂, substituted with 0-3 groups independently selected from R¹³;
   vii) a 5-6 membered heteroaryl having 1 to 3 ring members independently selected from N, NR⁷, O or S substituted with 0-3 groups independently selected from R¹³;
   and
   viii) a 9-10 membered heteroaryl having 1 to 4 ring members independently selected from N, NR⁷, N⁺O⁻, O, or S substituted with 0-3 groups independently selected from R¹³;
each R¹² is independently selected from —CN, —CD₃, —OCD₃, —OH, —NR⁷R⁸, —NR⁷C(═O)R⁸, —NR⁷S(═O)₂R⁸, —C(═O)NR⁷R⁸, —S(═O)₂R⁸, —S(═O)₂R¹⁰, halo, C₁-C₈haloalkoxy, C₁-C₈-alkoxy, C₁-C₈haloalkyl or —C₁-C₈alkyl;
each R¹³ is independently selected from —C₁-C₈alkyl, spiro attached C₃-C₈cycloalkyl, —C₁-C₈-alkoxy, —OH, halo, —C(═O)R⁷, —NR⁷R⁸, —C(═O)NR⁷R⁸, —NR⁷C(═O)R⁸, —NR⁷S(═O)₂R⁸, —S(═O)₂R⁸, NO₂, CN or C₁-C₈haloalkyl;
each m is independently selected from 1, 2, 3 or 4;
each n is independently selected from 0, 1, 2 or 3; and
each p is independently selected from 0, 1, 2 or 3.

Embodiment 2. The compound of Embodiment 1 having the structure of Formula (II), or a stereoisomer thereof or pharmaceutically acceptable salt thereof,

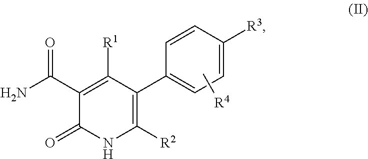

where R¹, R², R³ and R⁴ are as described herein.

Embodiment 3. The compound of Embodiment 1 or Embodiment 2, wherein
R¹ is H;
R² is —C₁-C₈haloalkyl;
R³ is L₁R⁵ or L₂R;
R⁴ is halo, CN, C₁-C₈alkyl, C₁-C₈alkoxy or C₃-C₈cycloalkyl;
L₁ is a —CH₂—, —OCH₂—, —O—, —CH₂O—, —O(CH₂)ₘ—, —CH(R⁷)—, —OCH(R⁷)—, —CH(R¹⁰)—, —OCH(R¹⁰)—, —CF₂CH₂—, —OCF₂—, —OCH₂C(═O)—, —OCH₂C(═O)NH—, or —OCH₂CH(OH)CH₂—;
L₂ is a bond, —CH₂—, —OCH₂—, —O(CH₂)ₘ—, —CH(R⁷)—, —OCH(R¹⁰)—, —OCH(R¹⁰)CH₂— or —OCH₂C(═O)—;
L₃ is a bond, —CH₂—, —OCH₂—, —O—, —NH—, —NH(CH₂)ₘ—, —CH₂OCH₂— or —CH₂O—;
L₄ is CH₂—, —(CH₂)ₘ—, —C(R⁷)₂—, —CF₂—, —OCH₂—, —O(CH₂)ₘ—, —CH₂O—, —CH₂OCH₂—, —CH₂OCH₂C(R⁷)₂— or —NH(CH₂)ₘ—;
R⁵ is selected from the group consisting of
   i) phenyl substituted with 0-3 groups independently selected from R⁹;
   ii) C₃-C₈cycloalkyl substituted with 0-3 groups independently selected from R⁹;
   iii) a 4-8 membered monocyclic heterocycloalkyl group having 1 to 3 ring members independently selected from N, NR⁷, O, S, C═O or S(═O)₂, substituted with 0-3 groups independently selected from R⁹;
   iv) a 9-12 membered heterocyclyl group having 1 to 4 ring members independently selected from N, NR⁷, O, or S, substituted with 0-3 groups independently selected from R⁹;
   v) a 7-8 membered bridged-bicyclic heterocycloalkyl group having 1 to 2 ring members independently selected from N, NR⁷, O or S, substituted with 0-3 groups independently selected from R⁹;
   vi) a 6-8 membered fused bicyclic heterocycloalkyl group having 1 to 2 ring members independently selected from N, NR⁷, O or S, substituted with 0-3 groups independently selected from R⁹;
   vii) a 5-8 membered heteroaryl having 1 to 3 ring members independently selected from N, NR⁷, O or S, substituted with 0-3 groups independently selected from R⁹;
   and
   viii) a 9-10 membered heteroaryl having 1 to 4 ring members independently selected from N, NR⁷, N⁺O⁻, O, or S, substituted with 0-3 groups independently selected from R⁹;
R⁶ is —CN, —OH, —NR⁷R⁸, —NR⁷C(═O)R⁸, —NR⁷S(═O)₂R⁸, —C(═O)NR⁷R⁸, —S(═O)₂R⁸, halo, C₁-C₈haloalkoxy, C₁-C₈-alkoxy, —C₁-C₈alkyl or —C₂-C₈alkylene;
each R⁷ is independently selected from H or —C₁-C₈alkyl;
R⁸ is H or —C₁-C₈alkyl;
each R⁹ is independently selected from —C₁-C₈alkyl, —C₁-C₈-alkoxy, C₁-C₈haloalkyl, spiro attached C₃-C₈cycloalkyl, R¹⁰, —OH, CN, halo, —C(═O)R⁷, —NR⁷R⁸, —C(═O)NR⁷R⁸, —NR⁷C(═O)R⁸, —C(═O)OR⁸, —NR⁷S(═O)₂R⁸, —S(═O)₂R⁸, —S(═O)₂R¹⁰, NO₂, CN, L₃R¹¹, L₄R¹² or a spiro attached 4-8 membered monocyclic heterocycloalkyl group having 1 to 2 ring members independently selected from N, NR⁷, O, or S;
R¹⁰ is a C₃-C₈cycloalkyl, a 4-8 membered monocyclic heterocycloalkyl group having 1 to 2 ring members independently selected from N, NR⁷, O or S, or a 5-6 membered heteroaryl having 1 to 2 ring members independently selected from N, NR⁷, O, or S;
each R¹¹ is independently selected from the group consisting of
   i) phenyl substituted with 0-3 groups independently selected from R¹³;
   ii) C₃-C₈cycloalkyl substituted with 0-3 groups independently selected from R¹³;
   iii) a 4-8 membered monocyclic heterocycloalkyl group having 1 to 3 ring members independently selected from N, NR⁷, O or S, substituted with 0-3 groups independently selected from R¹³;
   and
   iv) a 5-6 membered heteroaryl having 1 to 3 ring members independently selected from N, NR⁷, O or S substituted with 0-3 groups independently selected from R¹³;
each R¹² is independently selected from —CN, —CD₃, —OCD₃, —OH, —NR⁷R⁸, —NR⁷C(═O)R⁸, —NR⁷S(═O)₂R⁸, —C(═O)NR⁷R⁸, —S(═O)₂R⁸, —S(═O)₂R¹⁰, halo, C₁-C₈haloalkoxy, C₁-C₈haloalkyl, —C₁-C₈alkyl or C₁-C₈-alkoxy;
each R¹³ is independently selected from —C₁-C₈alkyl, —C₁-C₈-alkoxy, —OH or halo;
each m is independently selected from 1, 2 or 3,
and
each p is independently selected from 0 or 1.

Embodiment 4. The compound of any one of Embodiments 1 to 3, having the Formula (III), or a stereoisomer thereof or a pharmaceutically acceptable salt thereof,

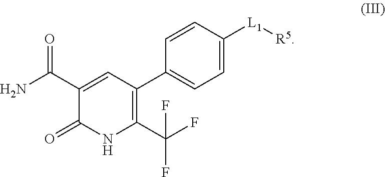

Embodiment 5. The compound of any one of Embodiments 1 to 4, having the Formula (IV), or a stereoisomer thereof or a pharmaceutically acceptable salt thereof,

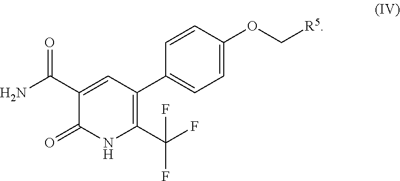

Embodiment 6. The compound of any one of Embodiments 1 to 5, or a stereoisomer thereof or a pharmaceutically acceptable salt thereof, wherein
R⁵ is selected from the group consisting of
   i) phenyl substituted with 0-3 groups independently selected from R⁹;
   ii) cyclopropyl, cyclobutyl or cyclohexyl, each of which is substituted with 0-3 groups independently selected from R⁹;
   iii) morpholinyl, pyrrolidinyl, thiomorpholinyl, oxetanyl, oxazepanyl, azetidinyl, pyrrolidinyl, piperidinyl, oxomorpholinyl, dioxidothiomorpholinyl, tetrahydropyranyl, tetrahydrofuranyl, dioxanyl, dioxepanyl, oxopyrrolidinyl, or oxooxazolidinyl, each of which is substituted with 0-3 groups independently selected from R⁹;
   iv) benzo[d][1,3]dioxolyl, benzo[b][1,4]dioxepinyl, tetrahydrobenzo[d]isoxazolyl, dihydrobenzofuranyl, tetrahydropyrano[3,4-c]pyrazolyl, chromanyl or 6,7-dihydro-4H-[1,2,3]triazolo[5,1-c][1,4]oxazinyl, each of which is substituted with 0-3 groups independently selected from R⁹;
   v) 8-oxa-3-azabicyclo[3.2.1]octanyl, oxabicyclo[2.2.1]heptanyl, or 3,8-dioxabicyclo[3.2.1]octanyl, each of which is substituted with 0-3 groups independently selected from R⁹;
   vi) 3-azabicyclo[3.1.0]hexanyl or 2,5-dioxabicyclo[4.1.0]heptanyl, each of which is substituted with 0-3 groups independently selected from R⁹;
   vii) oxazolyl, isoxazolyl, pyrazolyl, thiophenyl, thiazolyl, thiadiazolyl, imidazolyl, pyridinyl, pyrimidinyl, pyridazinyl or pyrazinyl, each of which is substituted with 0-3 groups independently selected from R⁹;
   and
   viii) benzo[c][1,2,5]oxadiazolyl, indazolyl, tetrazolo[1,5-a]pyridinyl, imidazo[1,2-a]pyridinyl, imidazo[4,5-b]pyridinyl, triazolo[4,3-a]pyrimidinyl, quinoxalinyl, quinolinyl, or isoquinolinyl, each of which is substituted with 0-3 groups independently selected from R⁹.

Embodiment 7. The compound of any one of Embodiments 1 to 6 having the Formula (V), or a stereoisomer thereof or a pharmaceutically acceptable salt thereof,

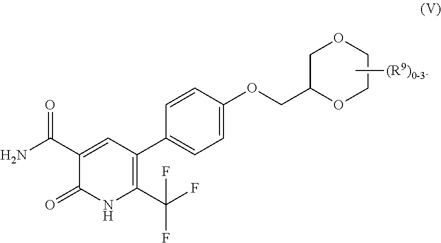

Embodiment 8. The compound of any one of Embodiments 1 to 7 having the Formula (V-a), Formula (V-b) or Formula (V-c), or a stereoisomer thereof or a pharmaceutically acceptable salt thereof,

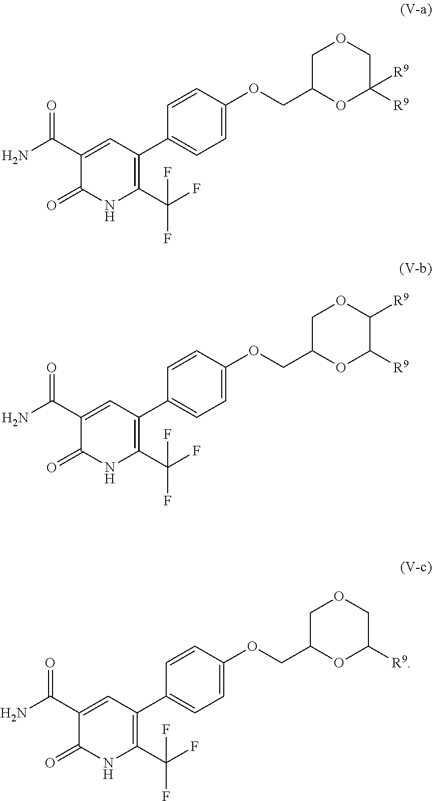

Embodiment 9. The compound of any one of Embodiments 1 to 8 having the Formula (V-a1) or Formula (V-a2), or a pharmaceutically acceptable salt thereof,

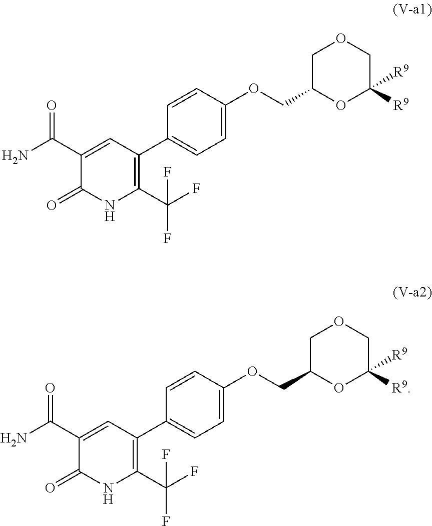

Embodiment 10. The compound of any one of Embodiments 1 to 9, wherein each R⁹ is independently selected from methyl, ethyl, isopropyl, neopentyl, methoxy, ethoxy, propoxy, isopropoxy, —CH₂F, —CHF₂, —CF₃, R¹⁰, —OH, CN, F, C₁, Br, —C(═O)CH₃, —NH₂, —NHCH₃, —NHCH₂CH₃, —NHCH₂CH₂CH₃, —NH(CH₂)SCH₃, —N(CH₃)₂, —C(═O)NH₂, —C(═O)N(CH₃)₂, —C(═O)NHCH₃, —NHC(═O)CH₃, —NHC(═O)OC(CH₃)₃, —C(═O)OC(CH₃)₃, —NHS(═O)₂CH₃, —S(═O)₂CH₃, —S(═O)₂CH(CH₃)₂, —S(═O)₂CH₂CH(CH₃)₂, —S(═O)₂— cyclopropyl, NO₂, CN, L₃R¹¹, L₄R¹², cyclopropyl, cyclopentyl, spiro attached cyclopropyl, spiro attached oxetanyl, spiro attached dioxepanyl or spiro attached tetrahydrofuranyl.

Embodiment 11. The compound of any one of Embodiments 1 to 9, wherein each R⁹ is independently selected from —C₁-C₈alkyl, —C₁-C₈alkoxy, halo, C₁-C₈haloalkyl, spiro attached C₃-C₈cycloalkyl, R¹⁰, L₃R¹, L₄R¹² or a spiro attached 4-8 membered monocyclic heterocycloalkyl group having 1 to 2 ring members independently selected from N, NR⁷, O, or S.

Embodiment 12. The compound of any one of Embodiments 1 to 11, wherein each R⁹ is independently selected from methyl, F, methoxy, —CH₂F, —CHF₂, L₃R¹¹, L₄R¹², —C(═O)N(CH₃)₂, cyclopropyl, cyclopentyl, spiro attached cyclopropyl, spiro attached oxetanyl, spiro attached dioxepanyl or spiro attached tetrahydrofuranyl.

Embodiment 13. The compound of any one of Embodiments 1 to 12, wherein each R⁹ is independently selected from methyl, L₃R¹¹ or L₄R¹².

Embodiment 14. The compound of any one of Embodiments 1 to 13, wherein each R⁹ is independently selected from methyl or L₃R¹¹, and wherein
L₃ is a bond, —CH₂—, —OCH₂—, —O—, —NH—, —NH(CH₂)ₘ—, —CH₂OCH₂—, —CH₂O—, —O(CH₂)ₘ—, —CH(R⁷)—, —OCH(R⁷)—, —CH(R¹⁰)—, —CF₂CH₂—, —C(R⁷)₂—, or —OC(R⁷)₂—;
R¹¹ is phenyl, tetrahydrofuranyl, cyclobutyl, cyclopropyl, cyclopentyl, oxetanyl, azetidinyl, pyrrolyl or pyrazolyl, each of which is substituted with 0 to 2 R¹³ groups,
and
each R¹³ is independently selected from F, OH, methoxy or methyl.

Embodiment 15. The compound of any one of Embodiments 1 to 14, wherein each R⁹ is independently selected from methyl or L₃R¹¹, and wherein
L₃ is bond, —CH₂, —OCH₂—, —O—, —NH—, —NHCH₂—, —CH₂OCH₂— or —CH₂O—;
R¹¹ is phenyl, tetrahydrofuranyl, cyclobutyl, cyclopropyl, cyclopentyl, oxetanyl, azetidinyl, pyrrolyl or pyrazolyl, each of which is substituted with 0 to 2 R¹³ groups,
and
each R¹³ is independently selected from F, OH, methoxy or methyl.

Embodiment 16. The compound of any one of Embodiments 1 to 13, wherein each R⁹ is independently selected from methyl or L₄R¹², and wherein
L₄ is —OCH2CH₂—, —NHCH₂CH₂—, —CH₂—, —CH₂O—, —CH₂CH₂—, —C(CH₃)₂—, —C(CH₂CH₃)₂—, —CF₂—, —CH₂OCH₂—, —CH₂OCH₂CH₂— or —CH₂OCH₂C(CH₃)₂—;
and
R¹² is OH, CN, CD₃, OCD₃, F, Cl, —OCH₂F, —OCHF₂, OCF₃, OCH₂CH₂F, OCH₂CHF₂, OCH₂CHF₃, —CH₂F, CHF₂, CF₃, —CH₂CH₂F, —CH₂CHF₂, —CH₂CHF₃, methoxy, ethoxy, isopropoxy, CN, methyl, ethyl, isopropyl, —S(═O)₂CH₃, —S(═O)₂-cyclopropyl or —N(CH₃)₂.

Embodiment 17. The compound of any one of Embodiments 1 to 13 having the Formula (V-b1), Formula (V-b2), Formula (V-b3) or Formula (V-b4), or a pharmaceutically acceptable salt thereof,

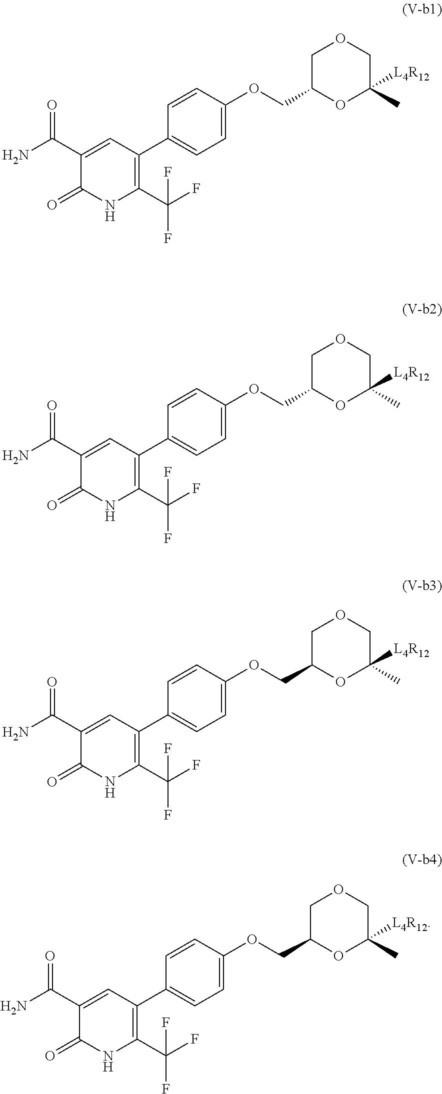

Embodiment 18. The compound of any one of Embodiments 1 to 13 having the Formula (V-c1), Formula (V-c2), Formula (V-c3) or Formula (V-c4), or a pharmaceutically acceptable salt thereof,

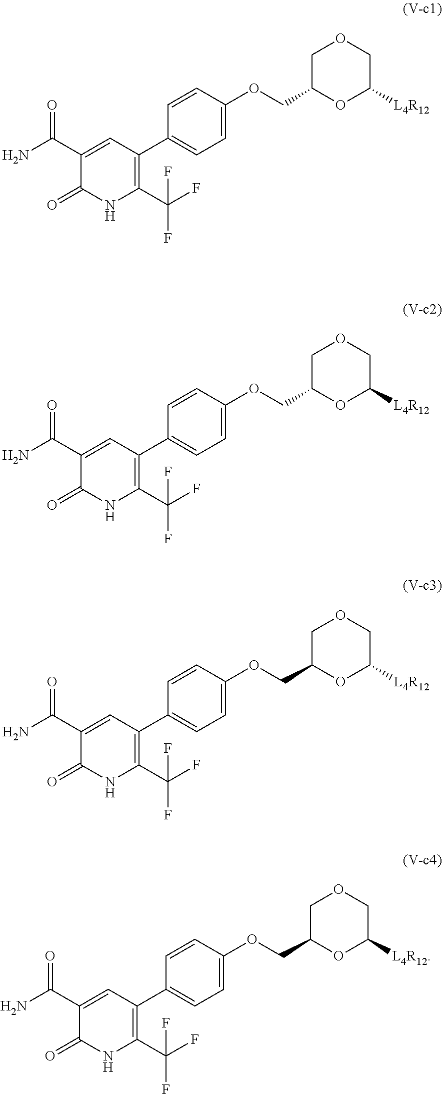

Embodiment 19. The compound of any one of Embodiments 1 to 18, wherein
L₄ is —OCH₂CH₂—, —NHCH₂CH₂—, —CH₂—, —CH₂O—, —CH₂CH₂—, —C(CH₃)₂—, —C(CH₂CH₃)₂—, —CF₂—, —CH₂OCH₂—, —CH₂OCH₂CH₂— or —CH₂OCH₂C(CH₃)₂—;
R¹² is OH, CN, CD₃, OCD₃, F, C₁, —OCH₂F, —OCHF₂, OCF₃, OCH₂CH₂F, OCH₂CHF₂, OCH₂CHF₃, —CH₂F, CHF₂, CF₃, —CH₂CH₂F, —CH₂CHF₂, —CH₂CHF₃, methoxy, ethoxy, isopropoxy, CN, methyl, ethyl, isopropyl, —S(═O)₂CH₃, —S(═O)₂-cyclopropyl or —N(CH₃)₂.

Embodiment 20. The compound of any one of Embodiments 1 to 19, wherein L₄ is a bond, —CH₂—, —(CH₂)ₘ—, —C(R⁷)₂—, —CF₂—, —OCH₂—, —O(CH₂)ₘ—, —CH₂O—, —CH₂OCH₂—, or —CH₂OCH₂C(R⁷)₂—.

Embodiment 21. The compound of any one of Embodiments 1 to 20, wherein L₄ is a bond, —CH₂—, —(CH₂)₂—, —C(CH₃)₂—, —C(CH₂CH₃)₂—, —CF₂—, —CH₂O—, —CH₂OCH₂—, —CH₂OCH₂C(CH₃)₂— or —CH₂OCH₂CH₂.

Embodiment 22. The compound of any one of Embodiments 1 to 21, wherein L₄ is —CH₂—, or —CH₂O—.

Embodiment 23. The compound of any one of Embodiments 1 to 22 having the Formula (V-d1), Formula (V-d2), Formula (V-d3) or Formula (V-d4) or a pharmaceutically acceptable salt thereof,

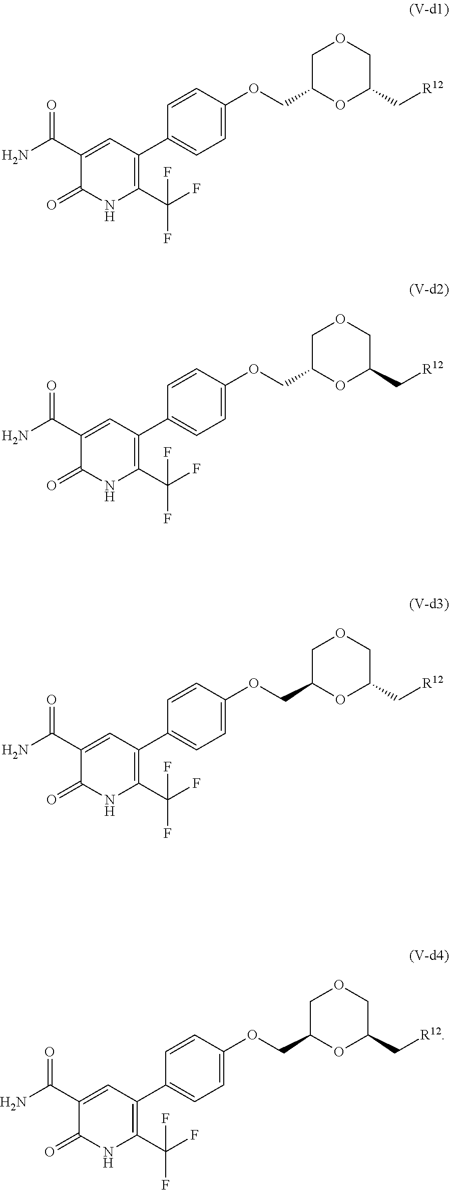

Embodiment 24. The compound of any one of Embodiments 1 to 13 having the Formula (V-e1), Formula (V-e2), Formula (V-e3) or Formula (V-e4), or a pharmaceutically acceptable salt thereof,

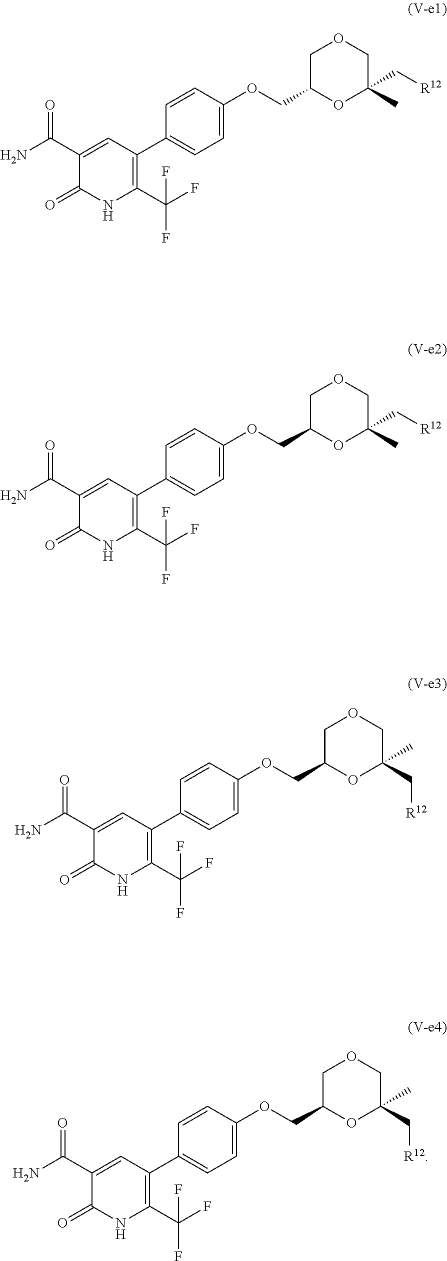

Embodiment 25. The compound of any one of Embodiments 1 to 24, wherein R¹² is CN, CD₃, OCD₃, OH, N(CH₃)₂, —S(O)₂CH₃, —S(O)₂-cyclopropyl, F, Cl, —OCHF₂, —OCHF₂, OCF₃, CH₂F, CHF₂, CF₃, -methyl, methoxy, ethoxy or isopropoxy

Embodiment 26. The compound of any one of Embodiments 1 to 25, wherein R¹² is methoxy, ethoxy or isopropoxy.

Embodiment 27. A compound of Embodiment 1, selected from:
5-(4-(morpholinomethyl)phenyl)-2-oxo-6-(trifluoromethyl)-1,2-dihydropyridine-3-carboxamide;
5-(4-(hydroxymethyl)phenyl)-2-oxo-6-(trifluoromethyl)-1,2-dihydropyridine-3-carboxamide;
5-(4-hydroxyphenyl)-2-oxo-6-(trifluoromethyl)-1,2-dihydropyridine-3-carboxamide;
5-(4-(benzyloxy)phenyl)-2-oxo-6-(trifluoromethyl)-1,2-dihydropyridine-3-carboxamide;
2-oxo-5-(4-(pyrrolidin-1-ylmethyl)phenyl)-6-(trifluoromethyl)-1,2-dihydropyridine-3-carboxamide;
2-oxo-5-(4-phenoxyphenyl)-6-(trifluoromethyl)-1,2-dihydropyridine-3-carboxamide;
5-(3-aminophenyl)-2-oxo-6-(trifluoromethyl)-1,2-dihydropyridine-3-carboxamide;
5-(3-cyanophenyl)-2-oxo-6-(trifluoromethyl)-1,2-dihydropyridine-3-carboxamide;
5-(3-hydroxyphenyl)-2-oxo-6-(trifluoromethyl)-1,2-dihydropyridine-3-carboxamide;
5-(3-formamidophenyl)-2-oxo-6-(trifluoromethyl)-1,2-dihydropyridine-3-carboxamide;
5-(4-(methylsulfonamido)phenyl)-2-oxo-6-(trifluoromethyl)-1,2-dihydropyridine-3-carboxamide;
5-(4-(difluoromethoxy)phenyl)-2-oxo-6-(trifluoromethyl)-1,2-dihydropyridine-3-carboxamide;
5-(4-(methoxymethoxy)phenyl)-2-oxo-6-(trifluoromethyl)-1,2-dihydropyridine-3-carboxamide;
5-(3-fluoro-4-methoxyphenyl)-2-oxo-6-(trifluoromethyl)-1,2-dihydropyridine-3-carboxamide;
5-(4-(methoxymethyl)phenyl)-2-oxo-6-(trifluoromethyl)-1,2-dihydropyridine-3-carboxamide;
2-oxo-5-phenyl-6-(trifluoromethyl)-1,2-dihydropyridine-3-carboxamide;
5-(4-isopropoxyphenyl)-2-oxo-6-(trifluoromethyl)-1,2-dihydropyridine-3-carboxamide;
5-(3-chloro-4-methoxyphenyl)-2-oxo-6-(trifluoromethyl)-1,2-dihydropyridine-3-carboxamide;
5-(4-(methylcarbamoyl)phenyl)-2-oxo-6-(trifluoromethyl)-1,2-dihydropyridine-3-carboxamide;
5-(4-(cyanomethyl)phenyl)-2-oxo-6-(trifluoromethyl)-1,2-dihydropyridine-3-carboxamide;
5-(4-(3-(dimethylamino)propoxy)phenyl)-2-oxo-6-(trifluoromethyl)-1,2-dihydropyridine-3-carboxamide;
5-(3-(cyanomethyl)phenyl)-2-oxo-6-(trifluoromethyl)-1,2-dihydropyridine-3-carboxamide;
5-(3-(morpholinomethyl)phenyl)-2-oxo-6-(trifluoromethyl)-1,2-dihydropyridine-3-carboxamide;
2-oxo-5-(m-tolyl)-6-(trifluoromethyl)-1,2-dihydropyridine-3-carboxamide;
5-(3-(hydroxymethyl)phenyl)-2-oxo-6-(trifluoromethyl)-1,2-dihydropyridine-3-carboxamide;
2-oxo-5-(p-tolyl)-6-(trifluoromethyl)-1,2-dihydropyridine-3-carboxamide;
5-(4-((3-methylmorpholino)methyl)phenyl)-2-oxo-6-(trifluoromethyl)-1,2-dihydropyridine-3-carboxamide;
2-oxo-5-(4-(thiomorpholinomethyl)phenyl)-6-(trifluoromethyl)-1,2-dihydropyridine-3-carboxamide;
5-(4-((2-methylmorpholino)methyl)phenyl)-2-oxo-6-(trifluoromethyl)-1,2-dihydropyridine-3-carboxamide;
5-(4-((7-oxa-4-azaspiro[2.5]octan-4-yl)methyl)phenyl)-2-oxo-6-(trifluoromethyl)-1,2-dihydropyridine-3-carboxamide;
5-(4-(((1R,5S)-8-oxa-3-azabicyclo[3.2.1]octan-3-yl)methyl)phenyl)-2-oxo-6-(trifluoromethyl)-1,2-dihydropyridine-3-carboxamide;
2-oxo-5-(4-((3-(pyridin-3-yl)morpholino)methyl)phenyl)-6-(trifluoromethyl)-1,2-dihydropyridine-3-carboxamide;
5-(4-(1-morpholinoethyl)phenyl)-2-oxo-6-(trifluoromethyl)-1,2-dihydropyridine-3-carboxamide;
5-(4-((1,4-oxazepan-4-yl)methyl)phenyl)-2-oxo-6-(trifluoromethyl)-1,2-dihydropyridine-3-carboxamide;
5-(4-(((2S,6R)-2,8-dimethylmorpholino)methyl)phenyl)-2-oxo-6-(trifluoromethyl)-1,2-dihydropyridine-3-carboxamide;
5-(3-fluoro-4-(morpholinomethyl)phenyl)-2-oxo-6-(trifluoromethyl)-1,2-dihydropyridine-3-carboxamide;
5-(4-(1-hydroxyethyl)phenyl)-2-oxo-6-(trifluoromethyl)-1,2-dihydropyridine-3-carboxamide;
5-(4-((3-methoxyazetidin-1-yl)methyl)phenyl)-2-oxo-6-(trifluoromethyl)-1,2-dihydropyridine-3-carboxamide;
5-(4-((2,2-dimethylmorpholino)methyl)phenyl)-2-oxo-6-(trifluoromethyl)-1,2-dihydropyridine-3-carboxamide;
(S)-5-(4-((3-methoxypyrrolidin-1-yl)methyl)phenyl)-2-oxo-6-(trifluoromethyl)-1,2-dihydropyridine-3-carboxamide;
(R)-5-(4-((3-methoxypyrrolidin-1-yl)methyl)phenyl)-2-oxo-6-(trifluoromethyl)-1,2-dihydropyridine-3-carboxamide;
5-(2-fluoro-4-(morpholinomethyl)phenyl)-2-oxo-6-(trifluoromethyl)-1,2-dihydropyridine-3-carboxamide;
5-(3-methyl-4-(morpholinomethyl)phenyl)-2-oxo-6-(trifluoromethyl)-1,2-dihydropyridine-3-carboxamide;
2-oxo-5-(4-(piperidin-1-ylmethyl)phenyl)-6-(trifluoromethyl)-1,2-dihydropyridine-3-carboxamide;
5-(4-((dimethylamino)methyl)phenyl)-2-oxo-6-(trifluoromethyl)-1,2-dihydropyridine-3-carboxamide;
2-oxo-5-(4-((3-oxomorpholino)methyl)phenyl)-6-(trifluoromethyl)-1,2-dihydropyridine-3-carboxamide;
5-(4-((1,1-dioxidothiomorpholino)methyl)phenyl)-2-oxo-6-(trifluoromethyl)-1,2-dihydropyridine-3-carboxamide;
5-(4-(morpholino(oxazol-5-yl)methyl)phenyl)-2-oxo-6-(trifluoromethyl)-1,2-dihydropyridine-3-carboxamide;
5-(4-(methylsulfonamidomethyl)phenyl)-2-oxo-6-(trifluoromethyl)-1,2-dihydropyridine-3-carboxamide;
5-(4-((4-hydroxytetrahydro-2H-pyran-4-yl)methyl)phenyl)-2-oxo-6-(trifluoromethyl)-1,2-dihydropyridine-3-carboxamide;
5-(4-((4-fluorotetrahydro-2H-pyran-4-yl)methyl)phenyl)-2-oxo-6-(trifluoromethyl)-1,2-dihydropyridine-3-carboxamide;
2-oxo-5-(4-((tetrahydro-2H-pyran-4-yl)methyl)phenyl)-6-(trifluoromethyl)-1,2-dihydropyridine-3-carboxamide;
5-(4-(2-(1,4-dioxan-2-yl)-1,1-difluoroethyl)phenyl)-2-oxo-6-(trifluoromethyl)-1,2-dihydropyridine-3-carboxamide;
5-(4-((3-nitrobenzyl)oxy)phenyl)-2-oxo-6-(trifluoromethyl)-1,2-dihydropyridine-3-carboxamide;
5-(4-((1-acetylpiperidin-4-yl)methoxy)phenyl)-2-oxo-6-(trifluoromethyl)-1,2-dihydropyridine-3-carboxamide;
2-oxo-5-(4-((tetrahydro-2H-pyran-4-yl)methoxy)phenyl)-6-(trifluoromethyl)-1,2-dihydropyridine-3-carboxamide;
5-(4-((3-aminobenzyl)oxy)phenyl)-2-oxo-6-(trifluoromethyl)-1,2-dihydropyridine-3-carboxamide;
5-(4-(cyclohexylmethoxy)phenyl)-2-oxo-6-(trifluoromethyl)-1,2-dihydropyridine-3-carboxamide;
5-(4-((4-methoxybenzyl)oxy)phenyl)-2-oxo-6-(trifluoromethyl)-1,2-dihydropyridine-3-carboxamide;
5-(4-((4-(dimethylcarbamoyl)benzyl)oxy)phenyl)-2-oxo-6-(trifluoromethyl)-1,2-dihydropyridine-3-carboxamide;
5-(4-((3-methoxybenzyl)oxy)phenyl)-2-oxo-6-(trifluoromethyl)-1,2-dihydropyridine-3-carboxamide;
5-(4-((3-(methylcarbamoyl)benzyl)oxy)phenyl)-2-oxo-6-(trifluoromethyl)-1,2-dihydropyridine-3-carboxamide;
2-oxo-5-(4-((tetrahydro-2H-pyran-4-yl)oxy)phenyl)-6-(trifluoromethyl)-1,2-dihydropyridine-3-carboxamide;
5-(4-((3-(methylsulfonamido)benzyl)oxy)phenyl)-2-oxo-6-(trifluoromethyl)-1,2-dihydropyridine-3-carboxamide;
5-(4-((3-carbamoylbenzyl)oxy)phenyl)-2-oxo-6-(trifluoromethyl)-1,2-dihydropyridine-3-carboxamide;
2-oxo-5-(4-((3-(propylamino)benzyl)oxy)phenyl)-6-(trifluoromethyl)-1,2-dihydropyridine-3-carboxamide;
5-(4-((3-(dimethylcarbamoyl)benzyl)oxy)phenyl)-2-oxo-6-(trifluoromethyl)-1,2-dihydropyridine-3-carboxamide;
5-(4-((3-acetamidobenzyl)oxy)phenyl)-2-oxo-6-(trifluoromethyl)-1,2-dihydropyridine-3-carboxamide;
5-(4-((4-(methylcarbamoyl)benzyl)oxy)phenyl)-2-oxo-6-(trifluoromethyl)-1,2-dihydropyridine-3-carboxamide;
2-oxo-5-(4-(pyridin-4-ylmethoxy)phenyl)-6-(trifluoromethyl)-1,2-dihydropyridine-3-carboxamide;
5-(4-(benzo[c][1,2,5]oxadiazol-5-ylmethoxy)phenyl)-2-oxo-6-(trifluoromethyl)-1,2-dihydropyridine-3-carboxamide;
2-oxo-5-(4-(pyridin-3-ylmethoxy)phenyl)-6-(trifluoromethyl)-1,2-dihydropyridine-3-carboxamide;
2-oxo-5-(4-(pyridin-2-ylmethoxy)phenyl)-6-(trifluoromethyl)-1,2-dihydropyridine-3-carboxamide;
5-(4-(benzo[c][1,2,5]oxadiazol-4-ylmethoxy)phenyl)-2-oxo-6-(trifluoromethyl)-1,2-dihydropyridine-3-carboxamide;
5-(4-((3-(methylsulfonyl)benzyl)oxy)phenyl)-2-oxo-6-(trifluoromethyl)-1,2-dihydropyridine-3-carboxamide;
5-(4-((2-nitrobenzyl)oxy)phenyl)-2-oxo-6-(trifluoromethyl)-1,2-dihydropyridine-3-carboxamide;
5-(4-((4-cyanobenzyl)oxy)phenyl)-2-oxo-6-(trifluoromethyl)-1,2-dihydropyridine-3-carboxamide;
5-(4-((3-cyanobenzyl)oxy)phenyl)-2-oxo-6-(trifluoromethyl)-1,2-dihydropyridine-3-carboxamide;
2-oxo-6-(trifluoromethyl)-5-(4-((3-(trifluoromethylbenzyl)oxy)phenyl)-1,2-dihydropyridine-3-carboxamide;
(R or S)-2-oxo-5-(4-(1-(pyridin-3-yl)ethoxy)phenyl)-6-(trifluoromethyl)-1,2-dihydropyridine-3-carboxamide;
2-oxo-5-(4-(pyrimidin-5-ylmethoxy)phenyl)-6-(trifluoromethyl)-1,2-dihydropyridine-3-carboxamide;
5-(4-((1-methylpiperidin-4-yl)methoxy)phenyl)-2-oxo-6-(trifluoromethyl)-1,2-dihydropyridine-3-carboxamide;
2-oxo-5-(4-(pyridazin-4-ylmethoxy)phenyl)-6-(trifluoromethyl)-1,2-dihydropyridine-3-carboxamide;
5-(4-((1H-indazol-6-yl)methoxy)phenyl)-2-oxo-6-(trifluoromethyl)-1,2-dihydropyridine-3-carboxamide;
5-(4-((1H-indol-6-yl)methoxy)phenyl)-2-oxo-6-(trifluoromethyl)-1,2-dihydropyridine-3-carboxamide;
2-oxo-6-(trifluoromethyl)-5-(4-((4-(trifluoromethylbenzyl)oxy)phenyl)-1,2-dihydropyridine-3-carboxamide;
2-oxo-5-(4-((5-oxopyrrolidin-3-yl)methoxy)phenyl)-6-(trifluoromethyl)-1,2-dihydropyridine-3-carboxamide;
5-(4-((2-cyanobenzyl)oxy)phenyl)-2-oxo-6-(trifluoromethyl)-1,2-dihydropyridine-3-carboxamide;
2-oxo-5-(4-(tetrazolo[1,5-a]pyridin-7-ylmethoxy)phenyl)-6-(trifluoromethyl)-1,2-dihydropyridine-3-carboxamide;
5-(4-(imidazo[1,2-a]pyridin-7-ylmethoxy)phenyl)-2-oxo-6-(trifluoromethyl)-1,2-dihydropyridine-3-carboxamide;
5-(4-((1-methylpiperidin-3-yl)methoxy)phenyl)-2-oxo-6-(trifluoromethyl)-1,2-dihydropyridine-3-carboxamide;
5-(4-((4-fluoro-3-nitrobenzyl)oxy)phenyl)-2-oxo-6-(trifluoromethyl)-1,2-dihydropyridine-3-carboxamide;
8-((4-(5-carbamoyl-6-oxo-2-(trifluoromethyl)-1,8-dihydropyridin-3-yl)phenoxy)methyl)benzo[c][1,2,5]oxadiazole 1-oxide;
(R or S)-2-oxo-5-(4-(1-(pyrimidin-5-yl)ethoxy)phenyl)-6-(trifluoromethyl)-1,2-dihydropyridine-3-carboxamide;
(R or S)-5-(4-(1-(3-cyanophenyl)ethoxy)phenyl)-2-oxo-6-(trifluoromethyl)-1,2-dihydropyridine-3-carboxamide;
5-(4-((5-cyanopyridin-3-yl)methoxy)phenyl)-2-oxo-6-(trifluoromethyl)-1,2-dihydropyridine-3-carboxamide;
5-(4-(1-(benzo[c][1,2,5]oxadiazol-5-yl)ethoxy)phenyl)-2-oxo-6-(trifluoromethyl)-1,2-dihydropyridine-3-carboxamide;
2-oxo-5-(4-((tetrahydro-2H-pyran-3-yl)methoxy)phenyl)-6-(trifluoromethyl)-1,2-dihydropyridine-3-carboxamide;
5-(4-([1,2,4]triazolo[4,3-a]pyrimidin-6-ylmethoxy)phenyl)-2-oxo-6-(trifluoromethyl)-1,2-dihydropyridine-3-carboxamide;
5-(4-((5-chloro-2-fluoropyridin-3-yl)methoxy)phenyl)-2-oxo-6-(trifluoromethyl)-1,2-dihydropyridine-3-carboxamide;
5-(4-((5-chloropyridin-2-yl)methoxy)phenyl)-2-oxo-6-(trifluoromethyl)-1,2-dihydropyridine-3-carboxamide;
5-(4-((2-methoxypyridin-4-yl)methoxy)phenyl)-2-oxo-6-(trifluoromethyl)-1,2-dihydropyridine-3-carboxamide;
5-(4-((3,5-dimethylisoxazol-4-yl)methoxy)phenyl)-2-oxo-6-(trifluoromethyl)-1,2-dihydropyridine-3-carboxamide;
5-(4-((2-chloro-5-nitrobenzyl)oxy)phenyl)-2-oxo-6-(trifluoromethyl)-1,2-dihydropyridine-3-carboxamide;
5-(4-((1-methyl-3-(trifluoromethyl)-1H-pyrazol-4-yl)methoxy)phenyl)-2-oxo-6-(trifluoromethyl)-1,2-dihydropyridine-3-carboxamide;
2-oxo-5-(4-(thiophen-3-ylmethoxy)phenyl)-6-(trifluoromethyl)-1,2-dihydropyridine-3-carboxamide;
2-oxo-6-(trifluoromethyl)-5-(4-((8-(trifluoromethyl)pyridin-3-yl)methoxy)phenyl)-1,2-dihydropyridine-3-carboxamide;
5-(4-((3,4-difluorobenzyl)oxy)phenyl)-2-oxo-6-(trifluoromethyl)-1,2-dihydropyridine-3-carboxamide;
5-(4-((4-methoxy-3,5-dimethylpyridin-2-yl)methoxy)phenyl)-2-oxo-6-(trifluoromethyl)-1,2-dihydropyridine-3-carboxamide;
5-(4-((3-fluoropyridin-2-yl)methoxy)phenyl)-2-oxo-6-(trifluoromethyl)-1,2-dihydropyridine-3-carboxamide;
2-oxo-5-(4-(thiophen-2-ylmethoxy)phenyl)-6-(trifluoromethyl)-1,2-dihydropyridine-3-carboxamide;
5-(4-((2,5-difluorobenzyl)oxy)phenyl)-2-oxo-6-(trifluoromethyl)-1,2-dihydropyridine-3-carboxamide;
5-(4-((4-chloropyridin-2-yl)methoxy)phenyl)-2-oxo-6-(trifluoromethyl)-1,2-dihydropyridine-3-carboxamide;
5-(4-((2,4-dimethylthiazol-5-yl)methoxy)phenyl)-2-oxo-6-(trifluoromethyl)-1,2-dihydropyridine-3-carboxamide;
5-(4-((2-chloropyridin-3-yl)methoxy)phenyl)-2-oxo-6-(trifluoromethyl)-1,2-dihydropyridine-3-carboxamide;
2-oxo-5-(4-((5-(propylamino)pyridin-3-yl)methoxy)phenyl)-6-(trifluoromethyl)-1,2-dihydropyridine-3-carboxamide;
2-oxo-5-(4-(pyrimidin-2-ylmethoxy)phenyl)-6-(trifluoromethyl)-1,2-dihydropyridine-3-carboxamide;
5-(4-((2-morpholinopyrimidin-5-yl)methoxy)phenyl)-2-oxo-6-(trifluoromethyl)-1,2-dihydropyridine-3-carboxamide;
2-oxo-5-(4-(pyrazin-2-ylmethoxy)phenyl)-6-(trifluoromethyl)-1,2-dihydropyridine-3-carboxamide;
(R or S)-5-(4-(1-(5-cyanopyridin-3-yl)ethoxy)phenyl)-2-oxo-6-(trifluoromethyl)-1,2-dihydropyridine-3-carboxamide;
5-(4-((1-methyl-1H-pyrazol-5-yl)methoxy)phenyl)-2-oxo-6-(trifluoromethyl)-1,2-dihydropyridine-3-carboxamide;
5-(4-((4-methoxypyridin-2-yl)methoxy)phenyl)-2-oxo-6-(trifluoromethyl)-1,2-dihydropyridine-3-carboxamide;
5-(4-((8-morpholinopyridin-3-yl)methoxy)phenyl)-2-oxo-6-(trifluoromethyl)-1,2-dihydropyridine-3-carboxamide;
5-(4-((2-fluoropyridin-3-yl)methoxy)phenyl)-2-oxo-6-(trifluoromethyl)-1,2-dihydropyridine-3-carboxamide;
5-(4-((5-fluoro-2-methoxypyridin-4-yl)methoxy)phenyl)-2-oxo-6-(trifluoromethyl)-1,2-dihydropyridine-3-carboxamide;
2-oxo-5-(4-(quinoxalin-6-ylmethoxy)phenyl)-6-(trifluoromethyl)-1,2-dihydropyridine-3-carboxamide;
5-(4-((1-methyl-1H-indazol-4-yl)methoxy)phenyl)-2-oxo-6-(trifluoromethyl)-1,2-dihydropyridine-3-carboxamide;
5-(4-((4-(methylsulfonyl)benzyl)oxy)phenyl)-2-oxo-6-(trifluoromethyl)-1,2-dihydropyridine-3-carboxamide;
2-oxo-5-(4-(thiazol-5-ylmethoxy)phenyl)-6-(trifluoromethyl)-1,2-dihydropyridine-3-carboxamide;
5-(4-((4,8-dimethylpyridin-2-yl)methoxy)phenyl)-2-oxo-6-(trifluoromethyl)-1,2-dihydropyridine-3-carboxamide;
5-(4-((2-methoxypyridin-3-yl)methoxy)phenyl)-2-oxo-6-(trifluoromethyl)-1,2-dihydropyridine-3-carboxamide;
5-(4-(isoquinolin-7-ylmethoxy)phenyl)-2-oxo-6-(trifluoromethyl)-1,2-dihydropyridine-3-carboxamide;
2-oxo-5-(4-(quinolin-7-ylmethoxy)phenyl)-6-(trifluoromethyl)-1,2-dihydropyridine-3-carboxamide;
5-(4-((3-fluoropyridin-4-yl)methoxy)phenyl)-2-oxo-6-(trifluoromethyl)-1,2-dihydropyridine-3-carboxamide;
5-(4-((4,8-dimethylpyrimidin-2-yl)methoxy)phenyl)-2-oxo-6-(trifluoromethyl)-1,2-dihydropyridine-3-carboxamide;
5-(4-((1-methyl-1H-imidazol-2-yl)methoxy)phenyl)-2-oxo-6-(trifluoromethyl)-1,2-dihydropyridine-3-carboxamide;
5-(4-((2-morpholinopyridin-3-yl)methoxy)phenyl)-2-oxo-6-(trifluoromethyl)-1,2-dihydropyridine-3-carboxamide;
2-oxo-5-(4-(thiazol-2-ylmethoxy)phenyl)-6-(trifluoromethyl)-1,2-dihydropyridine-3-carboxamide;
5-(4-((3,4-dimethoxypyridin-2-yl)methoxy)phenyl)-2-oxo-6-(trifluoromethyl)-1,2-dihydropyridine-3-carboxamide;
5-(4-((1-methyl-1H-indazol-5-yl)methoxy)phenyl)-2-oxo-6-(trifluoromethyl)-1,2-dihydropyridine-3-carboxamide;
5-(4-((5-methoxypyridin-3-yl)methoxy)phenyl)-2-oxo-6-(trifluoromethyl)-1,2-dihydropyridine-3-carboxamide;
5-(4-((2,8-dimethylpyridin-4-yl)methoxy)phenyl)-2-oxo-6-(trifluoromethyl)-1,2-dihydropyridine-3-carboxamide;
5-(4-((8-chlorobenzo[d][1,3]dioxol-5-yl)methoxy)phenyl)-2-oxo-6-(trifluoromethyl)-1,2-dihydropyridine-3-carboxamide;
5-(4-((3,4-dihydro-2H-benzo[b][1,4]dioxepin-7-yl)methoxy)phenyl)-2-oxo-6-(trifluoromethyl)-1,2-dihydropyridine-3-carboxamide;
5-(4-((2-hydroxypyrimidin-5-yl)methoxy)phenyl)-2-oxo-6-(trifluoromethyl)-1,2-dihydropyridine-3-carboxamide;
5-(4-((2-(oxetan-3-ylmethoxy)pyrimidin-5-yl)methoxy)phenyl)-2-oxo-6-(trifluoromethyl)-1,2-dihydropyridine-3-carboxamide;
5-(4-((2-(2-hydroxyethoxy)pyrimidin-5-yl)methoxy)phenyl)-2-oxo-6-(trifluoromethyl)-1,2-dihydropyridine-3-carboxamide;
2-oxo-5-(4-((2-propoxypyrimidin-5-yl)methoxy)phenyl)-6-(trifluoromethyl)-1,2-dihydropyridine-3-carboxamide;
2-oxo-5-(4-((2-((tetrahydrofuran-3-yl)oxy)pyrimidin-5-yl)methoxy)phenyl)-6-(trifluoromethyl)-1,2-dihydropyridine-3-carboxamide;
5-(4-((2-(cyclopentyloxy)pyrimidin-5-yl)methoxy)phenyl)-2-oxo-6-(trifluoromethyl)-1,2-dihydropyridine-3-carboxamide;
5-(4-((2-(oxetan-2-ylmethoxy)pyrimidin-5-yl)methoxy)phenyl)-2-oxo-6-(trifluoromethyl)-1,2-dihydropyridine-3-carboxamide;
2-oxo-5-(4-((2-((tetrahydrofuran-3-yl)methoxy)pyrimidin-5-yl)methoxy)phenyl)-6-(trifluoromethyl)-1,2-dihydropyridine-3-carboxamide;
5-(4-((2-(methylamino)pyrimidin-5-yl)methoxy)phenyl)-2-oxo-6-(trifluoromethyl)-1,2-dihydropyridine-3-carboxamide;
5-(4-((2-(cyclopentylamino)pyrimidin-5-yl)methoxy)phenyl)-2-oxo-6-(trifluoromethyl)-1,2-dihydropyridine-3-carboxamide;
5-(4-((2-(dimethylamino)pyrimidin-5-yl)methoxy)phenyl)-2-oxo-6-(trifluoromethyl)-1,2-dihydropyridine-3-carboxamide;
5-(4-((2-((cyclopropylmethyl)amino)pyrimidin-5-yl)methoxy)phenyl)-2-oxo-6-(trifluoromethyl)-1,2-dihydropyridine-3-carboxamide;
5-(4-((2-((2-hydroxyethylamino)pyrimidin-5-yl)methoxy)phenyl)-2-oxo-6-(trifluoromethyl)-1,2-dihydropyridine-3-carboxamide;
5-(4-((2-(cyclopropylamino)pyrimidin-5-yl)methoxy)phenyl)-2-oxo-6-(trifluoromethyl)-1,2-dihydropyridine-3-carboxamide;
5-(4-((2-(heptylamino)pyrimidin-5-yl)methoxy)phenyl)-2-oxo-6-(trifluoromethyl)-1,2-dihydropyridine-3-carboxamide;
5-(4-((2-(oxetan-3-ylamino)pyrimidin-5-yl)methoxy)phenyl)-2-oxo-6-(trifluoromethyl)-1,2-dihydropyridine-3-carboxamide;
2-oxo-5-(4-((2-((tetrahydrofuran-3-yl)amino)pyrimidin-5-yl)methoxy)phenyl)-6-(trifluoromethyl)-1,2-dihydropyridine-3-carboxamide;
5-(4-((6-(cyclopentylamino)pyridin-3-yl)methoxy)phenyl)-2-oxo-6-(trifluoromethyl)-1,2-dihydropyridine-3-carboxamide;
5-(4-((6-((cyclopropylmethylamino)pyridin-3-yl)methoxy)phenyl)-2-oxo-6-(trifluoromethyl)-1,2-dihydropyridine-3-carboxamide;
5-(4-((6-(3,3-difluoroazetidin-1-yl)pyridin-3-yl)methoxy)phenyl)-2-oxo-6-(trifluoromethyl)-1,2-dihydropyridine-3-carboxamide;
5-(4-((6-(3-methoxyazetidin-1-yl)pyridin-3-yl)methoxy)phenyl)-2-oxo-6-(trifluoromethyl)-1,2-dihydropyridine-3-carboxamide;
5-(4-((2-cyclopropylpyrimidin-5-yl)methoxy)phenyl)-2-oxo-6-(trifluoromethyl)-1,2-dihydropyridine-3-carboxamide;
5-(4-((2-(cyclopentylmethyl)pyrimidin-5-yl)methoxy)phenyl)-2-oxo-6-(trifluoromethyl)-1,2-dihydropyridine-3-carboxamide;
5-(4-((2-neopentylpyrimidin-5-yl)methoxy)phenyl)-2-oxo-6-(trifluoromethyl)-1,2-dihydropyridine-3-carboxamide;
5-(4-((4-(cyclopentylamino)pyrimidin-5-yl)methoxy)phenyl)-2-oxo-6-(trifluoromethyl)-1,2-dihydropyridine-3-carboxamide;
5-(4-((3-(cyclopropylmethyl)-3H-imidazo[4,5-b]pyridin-6-yl)methoxy)phenyl)-2-oxo-6-(trifluoromethyl)-1,2-dihydropyridine-3-carboxamide;
5-(4-((8-chloropyridin-3-yl)methoxy)phenyl)-2-oxo-6-(trifluoromethyl)-1,2-dihydropyridine-3-carboxamide;
2-oxo-5-(4-((pyridin-3-yloxy)methyl)phenyl)-6-(trifluoromethyl)-1,2-dihydropyridine-3-carboxamide;
5-(4-((5-(furan-3-yl)pyridin-3-yl)methoxy)phenyl)-2-oxo-6-(trifluoromethyl)-1,2-dihydropyridine-3-carboxamide;
5-(4-((5-morpholinopyridin-3-yl)methoxy)phenyl)-2-oxo-6-(trifluoromethyl)-1,2-dihydropyridine-3-carboxamide;
2-oxo-5-(4-((5-(tetrahydrofuran-3-yl)pyridin-3-yl)methoxy)phenyl)-6-(trifluoromethyl)-1,2-dihydropyridine-3-carboxamide;
5-(4-((5-(3-hydroxyoxetan-3-yl)pyridin-3-yl)methoxy)phenyl)-2-oxo-6-(trifluoromethyl)-1,2-dihydropyridine-3-carboxamide;
5-(4-((5-(3-fluorooxetan-3-yl)pyridin-3-yl)methoxy)phenyl)-2-oxo-6-(trifluoromethyl)-1,2-dihydropyridine-3-carboxamide;
2-oxo-5-(4-((3-(pyridin-3-yl)oxetan-3-yl)oxy)phenyl)-6-(trifluoromethyl)-1,2-dihydropyridine-3-carboxamide;
5-(4-(cyclopropyl(pyridin-3-yl)methoxy)phenyl)-2-oxo-6-(trifluoromethyl)-1,2-dihydropyridine-3-carboxamide;
2-oxo-5-(4-((1-(pyridin-3-yl)pent-4-en-1-yl)oxy)phenyl)-6-(trifluoromethyl)-1,2-dihydropyridine-3-carboxamide;
5-(4-(2-methoxy-1-(pyridin-3-yl)ethoxy)phenyl)-2-oxo-6-(trifluoromethyl)-1,2-dihydropyridine-3-carboxamide;
5-(4-(2-hydroxy-1-(pyrazin-2-yl)ethoxy)phenyl)-2-oxo-6-(trifluoromethyl)-1,2-dihydropyridine-3-carboxamide;
5-(4-((4-acetylmorpholin-2-yl)methoxy)phenyl)-2-oxo-6-(trifluoromethyl)-1,2-dihydropyridine-3-carboxamide;
5-(4-(morpholin-2-ylmethoxy)phenyl)-2-oxo-6-(trifluoromethyl)-1,2-dihydropyridine-3-carboxamide;
tert-butyl 2-((4-(5-carbamoyl-6-oxo-2-(trifluoromethyl)-1,8-dihydropyridin-3-yl)phenoxy)methyl)morpholine-4-carboxylate;
5-(4-((4-(isopropylsulfonyl)morpholin-2-yl)methoxy)phenyl)-2-oxo-6-(trifluoromethyl)-1,2-dihydropyridine-3-carboxamide;
5-(4-((4-(2,2-difluoroethyl)morpholin-2-yl)methoxy)phenyl)-2-oxo-6-(trifluoromethyl)-1,2-dihydropyridine-3-carboxamide;
5-(4-((4-(2-methoxyethyl)morpholin-2-yl)methoxy)phenyl)-2-oxo-6-(trifluoromethyl)-1,2-dihydropyridine-3-carboxamide;
5-(4-((4-(cyclopropylmethyl)morpholin-2-yl)methoxy)phenyl)-2-oxo-6-(trifluoromethyl)-1,2-dihydropyridine-3-carboxamide;
5-(4-((1,4-oxazepan-2-yl)methoxy)phenyl)-2-oxo-6-(trifluoromethyl)-1,2-dihydropyridine-3-carboxamide;
5-(4-((4-(oxetan-3-yl)-1,4-oxazepan-2-yl)methoxy)phenyl)-2-oxo-6-(trifluoromethyl)-1,2-dihydropyridine-3-carboxamide;
5-(4-((4-(isopropylsulfonyl)-1,4-oxazepan-2-yl)methoxy)phenyl)-2-oxo-6-(trifluoromethyl)-1,2-dihydropyridine-3-carboxamide;
5-(4-((4-(isobutylsulfonyl)-1,4-oxazepan-2-yl)methoxy)phenyl)-2-oxo-6-(trifluoromethyl)-1,2-dihydropyridine-3-carboxamide;
5-(4-(2-morpholino-2-oxoethoxy)phenyl)-2-oxo-6-(trifluoromethyl)-1,2-dihydropyridine-3-carboxamide;
5-(4-(2-(butylamino)-2-oxoethoxy)phenyl-2-oxo-6-(trifluoromethyl)-1,2-dihydropyridine-3-carboxamide;
2-oxo-5-(4-(2-oxo-2-(pyridin-3-ylamino)ethoxy)phenyl)-6-(trifluoromethyl)-1,2-dihydropyridine-3-carboxamide;
5-(4-((4-methoxytetrahydro-2H-pyran-2-yl)methoxy)phenyl)-2-oxo-6-(trifluoromethyl)-1,2-dihydropyridine-3-carboxamide;
5-(4-(((2S,5S)-5-(hydroxymethyl)-1,4-dioxan-2-yl)methoxy)phenyl)-2-oxo-6-(trifluoromethyl)-1,2-dihydropyridine-3-carboxamide;
5-(4-((2,8-dioxa-5-azaspiro[3.5]nonan-6-yl)methoxy)phenyl)-2-oxo-6-(trifluoromethyl)-1,2-dihydropyridine-3-carboxamide;
5-(4-((5-methyl-2,8-dioxa-5-azaspiro[3.5]nonan-6-yl)methoxy)phenyl)-2-oxo-6-(trifluoromethyl)-1,2-dihydropyridine-3-carboxamide;
2-oxo-6-(trifluoromethyl)-5-(4-((4,5,5-trimethylmorpholin-3-yl)methoxy)phenyl)-1,2-dihydropyridine-3-carboxamide;
5-(4-(((5S)-4,5-dimethylmorpholin-3-yl)methoxy)phenyl)-2-oxo-6-(trifluoromethyl)-1,2-dihydropyridine-3-carboxamide;
5-(4-(((3S,5S)-5-cyclopropyl-4-methylmorpholin-3-yl)methoxy)phenyl)-2-oxo-6-(trifluoromethyl)-1,2-dihydropyridine-3-carboxamide;
5-(4-(((3R,5S)-5-cyclopropyl-4-methylmorpholin-3-yl)methoxy)phenyl)-2-oxo-6-(trifluoromethyl)-1,2-dihydropyridine-3-carboxamide;
5-(4-(((5S)-5-isopropyl-4-methylmorpholin-3-yl)methoxy)phenyl)-2-oxo-6-(trifluoromethyl)-1,2-dihydropyridine-3-carboxamide;
5-(4-((5-bromopyridin-3-yl)methoxy)phenyl)-2-oxo-6-(trifluoromethyl)-1,2-dihydropyridine-3-carboxamide;
5-(4-((5-chloropyridin-3-yl)methoxy)phenyl)-2-oxo-6-(trifluoromethyl)-1,2-dihydropyridine-3-carboxamide;
2-oxo-5-(4-(pyridazin-3-ylmethoxy)phenyl)-6-(trifluoromethyl)-1,2-dihydropyridine-3-carboxamide;
2-oxo-5-(4-(pyrimidin-4-ylmethoxy)phenyl)-6-(trifluoromethyl)-1,2-dihydropyridine-3-carboxamide;
5-(4-((8-chloropyrazin-2-yl)methoxy)phenyl)-2-oxo-6-(trifluoromethyl)-1,2-dihydropyridine-3-carboxamide;
5-(4-((1-ethyl-1H-pyrazol-5-yl)methoxy)phenyl)-2-oxo-6-(trifluoromethyl)-1,2-dihydropyridine-3-carboxamide;
5-(4-((1-(2-methoxyethyl)-1H-pyrazol-4-yl)methoxy)phenyl)-2-oxo-6-(trifluoromethyl)-1,2-dihydropyridine-3-carboxamide;
5-(4-((1-(2-methoxyethyl)-1H-pyrazol-5-yl)methoxy)phenyl)-2-oxo-6-(trifluoromethyl)-1,2-dihydropyridine-3-carboxamide;
5-(4-((1-methyl-1H-pyrazol-4-yl)methoxy)phenyl)-2-oxo-6-(trifluoromethyl)-1,2-dihydropyridine-3-carboxamide;
5-(4-((1-(cyclopropylmethyl)-1H-pyrazol-4-yl)methoxy)phenyl)-2-oxo-6-(trifluoromethyl)-1,2-dihydropyridine-3-carboxamide;
5-(4-((1-ethyl-1H-pyrazol-4-yl)methoxy)phenyl)-2-oxo-6-(trifluoromethyl)-1,2-dihydropyridine-3-carboxamide;
5-(4-(isoxazol-3-ylmethoxy)phenyl)-2-oxo-6-(trifluoromethyl)-1,2-dihydropyridine-3-carboxamide;
5-(4-((5-methylisoxazol-3-yl)methoxy)phenyl)-2-oxo-6-(trifluoromethyl)-1,2-dihydropyridine-3-carboxamide;
5-(4-(oxetan-2-ylmethoxy)phenyl)-2-oxo-6-(trifluoromethyl)-1,2-dihydropyridine-3-carboxamide;
5-(4-((3-fluorooxetan-3-yl)methoxy)phenyl)-2-oxo-6-(trifluoromethyl)-1,2-dihydropyridine-3-carboxamide;
5-(4-((3-methyl)oxetan-3-yl)methoxy)phenyl)-2-oxo-6-(trifluoromethyl)-1,2-dihydropyridine-3-carboxamide;
5-(4-((3-ethyl)oxetan-3-yl)methoxy)phenyl)-2-oxo-6-(trifluoromethyl)-1,2-dihydropyridine-3-carboxamide;
5-(4-((3-(cyanomethyl)oxetan-3-yl)methoxy)phenyl)-2-oxo-6-(trifluoromethyl)-1,2-dihydropyridine-3-carboxamide;
5-(4-((2-oxaspiro[3.3]heptan-6-yl)oxy)phenyl)-2-oxo-6-(trifluoromethyl)-1,2-dihydropyridine-3-carboxamide;
2-oxo-5-(4-((tetrahydrofuran-3-yl)methoxy)phenyl)-6-(trifluoromethyl)-1,2-dihydropyridine-3-carboxamide;
2-oxo-5-(4-((tetrahydrofuran-2-yl)methoxy)phenyl)-6-(trifluoromethyl)-1,2-dihydropyridine-3-carboxamide;
5-(4-((3-methyl-2-oxooxazolidin-5-yl)methoxy)phenyl)-2-oxo-6-(trifluoromethyl)-1,2-dihydropyridine-3-carboxamide;
5-(4-((3-ethyl-2-oxooxazolidin-5-yl)methoxy)phenyl)-2-oxo-6-(trifluoromethyl)-1,2-dihydropyridine-3-carboxamide;
5-(4-((3-methyl-2-oxooxazolidin-4-yl)methoxy)phenyl)-2-oxo-6-(trifluoromethyl)-1,2-dihydropyridine-3-carboxamide;
2-oxo-5-(4-((tetrahydro-2H-pyran-2-yl)methoxy)phenyl)-6-(trifluoromethyl)-1,2-dihydropyridine-3-carboxamide;
5-(4-((1,4-dioxan-2-yl)methoxy)phenyl)-2-oxo-6-(trifluoromethyl)-1,2-dihydropyridine-3-carboxamide;
5-(4-((1,4-dioxan-2-yl)methoxy)-3-fluorophenyl)-2-oxo-6-(trifluoromethyl)-1,2-dihydropyridine-3-carboxamide;
5-(4-((1,4-dioxan-2-yl)methoxy)-3-chlorophenyl)-2-oxo-6-(trifluoromethyl)-1,2-dihydropyridine-3-carboxamide;
5-(4-((1,4-dioxan-2-yl)methoxy)-3-cyanophenyl)-2-oxo-6-(trifluoromethyl)-1,2-dihydropyridine-3-carboxamide;
5-(4-((1,4-dioxan-2-yl)methoxy)-3-methylphenyl)-2-oxo-6-(trifluoromethyl)-1,2-dihydropyridine-3-carboxamide;
5-(4-((1,4-dioxan-2-yl)methoxy)-3-cyclopropylphenyl)-2-oxo-6-(trifluoromethyl)-1,2-dihydropyridine-3-carboxamide;
5-(4-((1,4-dioxan-2-yl)methoxy)-3-methoxyphenyl)-2-oxo-6-(trifluoromethyl)-1,2-dihydropyridine-3-carboxamide;
5-(4-((1,4-dioxan-2-yl)methoxy)-2-fluorophenyl)-2-oxo-6-(trifluoromethyl)-1,2-dihydropyridine-3-carboxamide;
5-(4-(morpholin-3-ylmethoxy)phenyl)-2-oxo-6-(trifluoromethyl)-1,2-dihydropyridine-3-carboxamide;
5-(4-((4-methylmorpholin-3-yl)methoxy)phenyl)-2-oxo-6-(trifluoromethyl)-1,2-dihydropyridine-3-carboxamide;
5-(4-((4-cyclopropylmorpholin-3-yl)methoxy)phenyl)-2-oxo-6-(trifluoromethyl)-1,2-dihydropyridine-3-carboxamide;
5-(4-((4-ethylmorpholin-3-yl)methoxy)phenyl)-2-oxo-6-(trifluoromethyl)-1,2-dihydropyridine-3-carboxamide;
2-oxo-5-(4-((5-oxomorpholin-3-yl)methoxy)phenyl)-6-(trifluoromethyl)-1,2-dihydropyridine-3-carboxamide;
5-(4-((4-methyl-5-oxomorpholin-3-yl)methoxy)phenyl)-2-oxo-6-(trifluoromethyl)-1,2-dihydropyridine-3-carboxamide;
5-(4-((1-(methylsulfonyl)cyclopropyl)methoxy)phenyl)-2-oxo-6-(trifluoromethyl)-1,2-dihydropyridine-3-carboxamide;
5-(4-((1-(cyclopropylsulfonyl)cyclopropyl)methoxy)phenyl)-2-oxo-6-(trifluoromethyl)-1,2-dihydropyridine-3-carboxamide;
5-(4-(2-methoxyethoxy)phenyl)-2-oxo-6-(trifluoromethyl)-1,2-dihydropyridine-3-carboxamide;
5-(4-(2-hydroxyethoxy)phenyl)-2-oxo-6-(trifluoromethyl)-1,2-dihydropyridine-3-carboxamide;
5-(4-(2-(methylsulfonyl)ethoxy)phenyl)-2-oxo-6-(trifluoromethyl)-1,2-dihydropyridine-3-carboxamide;
2-oxo-5-(4-(2-(2-oxooxazolidin-3-yl)ethoxy)phenyl)-6-(trifluoromethyl)-1,2-dihydropyridine-3-carboxamide;
5-(4-(2-(1H-imidazol-1-yl)ethoxy)phenyl)-2-oxo-6-(trifluoromethyl)-1,2-dihydropyridine-3-carboxamide;
5-(4-(2-morpholinoethoxy)phenyl)-2-oxo-6-(trifluoromethyl)-1,2-dihydropyridine-3-carboxamide;
5-(4-(2-hydroxy-3-morpholinopropoxy)phenyl)-2-oxo-6-(trifluoromethyl)-1,2-dihydropyridine-3-carboxamide;
5-(4-((1,3-dimethyl-1H-pyrazol-5-yl)methoxy)phenyl)-2-oxo-6-(trifluoromethyl)-1,2-dihydropyridine-3-carboxamide;
5-(4-((3-ethyl-1-methyl-1H-pyrazol-5-yl)methoxy)phenyl)-2-oxo-6-(trifluoromethyl)-1,2-dihydropyridine-3-carboxamide;
5-(4-((3-cyclopropyl-1-methyl-1H-pyrazol-5-yl)methoxy)phenyl)-2-oxo-6-(trifluoromethyl)-1,2-dihydropyridine-3-carboxamide;
2-oxo-6-(trifluoromethyl)-5-(4-((1,3,4-trimethyl-1H-pyrazol-5-yl)methoxy)phenyl)-1,2-dihydropyridine-3-carboxamide;
5-(4-((1-(cyclopropylmethyl)-1H-pyrazol-5-yl)methoxy)phenyl)-2-oxo-6-(trifluoromethyl)-1,2-dihydropyridine-3-carboxamide;
5-(4-(oxazol-4-ylmethoxy)phenyl)-2-oxo-6-(trifluoromethyl)-1,2-dihydropyridine-3-carboxamide;
5-(4-(oxazol-5-ylmethoxy)phenyl)-2-oxo-6-(trifluoromethyl)-1,2-dihydropyridine-3-carboxamide;
5-(4-((5-cyclopropylisoxazol-3-yl)methoxy)phenyl)-2-oxo-6-(trifluoromethyl)-1,2-dihydropyridine-3-carboxamide;
5-(4-((5-methylisoxazol-4-yl)methoxy)phenyl)-2-oxo-6-(trifluoromethyl)-1,2-dihydropyridine-3-carboxamide;
5-(4-((5-methyl-3-(pyridin-4-yl)isoxazol-4-yl)methoxy)phenyl)-2-oxo-6-(trifluoromethyl)-1,2-dihydropyridine-3-carboxamide;
5-(4-((2-methylthiazol-5-yl)methoxy)phenyl)-2-oxo-6-(trifluoromethyl)-1,2-dihydropyridine-3-carboxamide;
5-(4-((4-methylthiazol-5-yl)methoxy)phenyl)-2-oxo-6-(trifluoromethyl)-1,2-dihydropyridine-3-carboxamide;
5-(4-((4-methyl-1,2,3-thiadiazol-5-yl)methoxy)phenyl)-2-oxo-6-(trifluoromethyl)-1,2-dihydropyridine-3-carboxamide;
5-(4-((4-methylpyridin-3-yl)methoxy)phenyl)-2-oxo-6-(trifluoromethyl)-1,2-dihydropyridine-3-carboxamide;
5-(4-((4-cyclopropylpyridin-3-yl)methoxy)phenyl)-2-oxo-6-(trifluoromethyl)-1,2-dihydropyridine-3-carboxamide;
5-(4-((4-methoxypyridin-3-yl)methoxy)phenyl)-2-oxo-6-(trifluoromethyl)-1,2-dihydropyridine-3-carboxamide;
5-(4-((4-ethoxypyridin-3-yl)methoxy)phenyl)-2-oxo-6-(trifluoromethyl)-1,2-dihydropyridine-3-carboxamide;
5-(4-((5-methylpyridin-3-yl)methoxy)phenyl)-2-oxo-6-(trifluoromethyl)-1,2-dihydropyridine-3-carboxamide;
5-(4-((5-cyclopropylpyridin-3-yl)methoxy)phenyl)-2-oxo-6-(trifluoromethyl)-1,2-dihydropyridine-3-carboxamide;
5-(4-((4-isopropoxypyridin-3-yl)methoxy)phenyl)-2-oxo-6-(trifluoromethyl)-1,2-dihydropyridine-3-carboxamide;
5-(4-((4-methylpyrimidin-5-yl)methoxy)phenyl)-2-oxo-6-(trifluoromethyl)-1,2-dihydropyridine-3-carboxamide;
5-(4-((4-cyclopropylpyrimidin-5-yl)methoxy)phenyl)-2-oxo-6-(trifluoromethyl)-1,2-dihydropyridine-3-carboxamide;
5-(4-((8-methylpyrazin-2-yl)methoxy)phenyl)-2-oxo-6-(trifluoromethyl)-1,2-dihydropyridine-3-carboxamide;
5-(4-((8-cyclopropylpyrazin-2-yl)methoxy)phenyl)-2-oxo-6-(trifluoromethyl)-1,2-dihydropyridine-3-carboxamide;
5-(4-(imidazo[1,2-a]pyridin-2-ylmethoxy)phenyl)-2-oxo-6-(trifluoromethyl)-1,2-dihydropyridine-3-carboxamide;
5-(4-(imidazo[1,2-a]pyridin-3-ylmethoxy)phenyl)-2-oxo-6-(trifluoromethyl)-1,2-dihydropyridine-3-carboxamide;
2-oxo-5-(4-((4,5,6,7-tetrahydrobenzo[d]isoxazol-3-yl)methoxy)phenyl)-6-(trifluoromethyl)-1,2-dihydropyridine-3-carboxamide;
5-(4-((2,3-dihydrobenzofuran-2-yl)methoxy)phenyl)-2-oxo-6-(trifluoromethyl)-1,2-dihydropyridine-3-carboxamide;
5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carboxamide;
5-(4-((2-methyl-2,4,5,7-tetrahydropyrano[3,4-c]pyrazol-4-yl)methoxy)phenyl)-2-oxo-6-(trifluoromethyl)-1,2-dihydropyridine-3-carboxamide;
5-(4-((1-isopropyl-1,4,5,7-tetrahydropyrano[3,4-c]pyrazol-4-yl)methoxy)phenyl)-2-oxo-6-(trifluoromethyl)-1,2-dihydropyridine-3-carboxamide;
5-(4-((2-isopropyl-2,4,5,7-tetrahydropyrano[3,4-c]pyrazol-4-yl)methoxy)phenyl)-2-oxo-6-(trifluoromethyl)-1,2-dihydropyridine-3-carboxamide;
5-(4-(chroman-2-ylmethoxy)phenyl)-2-oxo-6-(trifluoromethyl)-1,2-dihydropyridine-3-carboxamide;
tert-butyl 3-((4-(5-carbamoyl-6-oxo-2-(trifluoromethyl)-1,6-dihydropyridin-3-yl)phenoxy)methyl)azetidine-1-carboxylate;
tert-butyl ((1r,3r)-3-((4-(5-carbamoyl-6-oxo-2-(trifluoromethyl)-1,6-dihydropyridin-3-yl)phenoxy)methyl)cyclobutyl)carbamate;
(S)-5-(4-((5,5-dimethyltetrahydrofuran-2-yl)methoxy)phenyl)-2-oxo-6-(trifluoromethyl)-1,2-dihydropyridine-3-carboxamide;
tert-butyl 6-((4-(5-carbamoyl-6-oxo-2-(trifluoromethyl)-1,6-dihydropyridin-3-yl)phenoxy)methyl)-3-azabicyclo[3.1.0]hexane-3-carboxylate;
tert-butyl 3-((4-(5-carbamoyl-6-oxo-2-(trifluoromethyl)-1,6-dihydropyridin-3-yl)phenoxy)methyl)pyrrolidine-1-carboxylate;
tert-butyl 3-((4-(5-carbamoyl-6-oxo-2-(trifluoromethyl)-1,6-dihydropyridin-3-yl)phenoxy)methyl)-4-methylpyrrolidine-1-carboxylate;
5-(4-((1-benzyl-3-cyanopyrrolidin-3-yl)methoxy)phenyl)-2-oxo-6-(trifluoromethyl)-1,2-dihydropyridine-3-carboxamide;
tert-butyl (2S,4R)-2-((4-(5-carbamoyl-6-oxo-2-(trifluoromethyl)-1,6-dihydropyridin-3-ylphenoxy)methyl)-4-fluoropyrrolidine-1-carboxylate;
tert-butyl (2S,4S)-2-((4-(5-carbamoyl-6-oxo-2-(trifluoromethyl)-1,6-dihydropyridin-3-ylphenoxy)methyl)-4-fluoropyrrolidine-1-carboxylate;
5-(4-((3-isopropyl-2-oxooxazolidin-5-yl)methoxy)phenyl)-2-oxo-6-(trifluoromethyl)-1,2-dihydropyridine-3-carboxamide;
5-(4-((3-(cyclopropylmethyl)-2-oxooxazolidin-5-yl)methoxy)phenyl)-2-oxo-6-(trifluoromethyl)-1,2-dihydropyridine-3-carboxamide;
5-(4-((3-ethyl-2-oxooxazolidin-4-yl)methoxy)phenyl)-2-oxo-6-(trifluoromethyl)-1,2-dihydropyridine-3-carboxamide;
5-(4-((3-isopropyl-2-oxooxazolidin-4-yl)methoxy)phenyl)-2-oxo-6-(trifluoromethyl)-1,2-dihydropyridine-3-carboxamide;
5-(4-((3-(cyclopropylmethyl)-2-oxooxazolidin-4-yl)methoxy)phenyl)-2-oxo-6-(trifluoromethyl)-1,2-dihydropyridine-3-carboxamide;
5-(4-((7-oxabicyclo[2.2.1]heptan-2-yl)methoxy)phenyl)-2-oxo-6-(trifluoromethyl)-1,2-dihydropyridine-3-carboxamide;
5-(4-((3,8-dioxabicyclo[3.2.1]octan-2-yl)methoxy)phenyl)-2-oxo-6-(trifluoromethyl)-1,2-dihydropyridine-3-carboxamide;
5-(4-((1,4-dioxepan-6-yl)methoxy)phenyl)-2-oxo-6-(trifluoromethyl)-1,2-dihydropyridine-3-carboxamide;
5-(4-((6-methyl-1,4-dioxepan-6-yl)methoxy)phenyl)-2-oxo-6-(trifluoromethyl)-1,2-dihydropyridine-3-carboxamide;
5-(4-((5-methyl-1,4-dioxan-2-yl)methoxy)phenyl)-2-oxo-6-(trifluoromethyl)-1,2-dihydropyridine-3-carboxamide;
5-(4-(((2R,6R)-6-cyclopropyl-1,4-dioxan-2-yl)methoxy)phenyl)-2-oxo-6-(trifluoromethyl)-1,2-dihydropyridine-3-carboxamide;
5-(4-(((2R,6S)-6-cyclopropyl-1,4-dioxan-2-yl)methoxy)phenyl)-2-oxo-6-(trifluoromethyl)-1,2-dihydropyridine-3-carboxamide;
5-(4-(((2R,6R)-6-methyl-1,4-dioxan-2-yl)methoxy)phenyl)-2-oxo-6-(trifluoromethyl)-1,2-dihydropyridine-3-carboxamide;
5-(4-(((2S,6R)-6-methyl-1,4-dioxan-2-yl)methoxy)phenyl)-2-oxo-6-(trifluoromethyl)-1,2-dihydropyridine-3-carboxamide;
5-(4-(((2S,6S)-6-methyl-1,4-dioxan-2-yl)methoxy)phenyl)-2-oxo-6-(trifluoromethyl)-1,2-dihydropyridine-3-carboxamide;
5-(4-(((2R,6S)-6-methyl-1,4-dioxan-2-yl)methoxy)phenyl)-2-oxo-6-(trifluoromethyl)-1,2-dihydropyridine-3-carboxamide;
5-(4-((6-cyclopentyl-1,4-dioxan-2-yl)methoxy)phenyl)-2-oxo-6-(trifluoromethyl)-1,2-dihydropyridine-3-carboxamide;
5-(4-((4,7-dioxaspiro[2.5]octan-5-yl)methoxy)phenyl)-2-oxo-6-(trifluoromethyl)-1,2-dihydropyridine-3-carboxamide;
5-(4-((5,5-difluorotetrahydro-2H-pyran-3-yl)methoxy)phenyl)-2-oxo-6-(trifluoromethyl)-1,2-dihydropyridine-3-carboxamide;
5-(4-((4,7-dioxaspiro[2.5]octan-6-yl)methoxy)phenyl)-2-oxo-6-(trifluoromethyl)-1,2-dihydropyridine-3-carboxamide;
5-(4-((5,5-dimethyl-1,4-dioxan-2-yl)methoxy)phenyl)-2-oxo-6-(trifluoromethyl)-1,2-dihydropyridine-3-carboxamide;
5-(4-((5-(fluoromethyl)-1,4-dioxan-2-yl)methoxy)phenyl)-2-oxo-6-(trifluoromethyl)-1,2-dihydropyridine-3-carboxamide;
5-(4-((5-(difluoromethyl)-1,4-dioxan-2-yl)methoxy)phenyl)-2-oxo-6-(trifluoromethyl)-1,2-dihydropyridine-3-carboxamide;
5-(4-((6-(difluoromethyl)-1,4-dioxan-2-yl)methoxy)phenyl)-2-oxo-6-(trifluoromethyl)-1,2-dihydropyridine-3-carboxamide;
5-(4-((6-(fluoromethyl)-1,4-dioxan-2-yl)methoxy)phenyl)-2-oxo-6-(trifluoromethyl)-1,2-dihydropyridine-3-carboxamide;
5-(4-((6-(1-methoxycyclopropyl)-1,4-dioxan-2-yl)methoxy)phenyl)-2-oxo-6-(trifluoromethyl)-1,2-dihydropyridine-3-carboxamide;
5-(4-((6-(2-hydroxypropan-2-yl)-1,4-dioxan-2-yl)methoxy)phenyl)-2-oxo-6-(trifluoromethyl)-1,2-dihydropyridine-3-carboxamide;
5-(4-((6-(1-methylcyclopropyl)-1,4-dioxan-2-yl)methoxy)phenyl)-2-oxo-6-(trifluoromethyl)-1,2-dihydropyridine-3-carboxamide;
5-(4-((6-(1,1-difluoroethyl)-1,4-dioxan-2-yl)methoxy)phenyl)-2-oxo-6-(trifluoromethyl)-1,2-dihydropyridine-3-carboxamide;
5-(4-((6-(ethoxymethyl)-1,4-dioxan-2-yl)methoxy)phenyl)-2-oxo-6-(trifluoromethyl)-1,2-dihydropyridine-3-carboxamide;
5-(4-((6-((cyclopropylmethoxy)methyl)-1,4-dioxan-2-yl)methoxy)phenyl)-2-oxo-6-(trifluoromethyl)-1,2-dihydropyridine-3-carboxamide;
5-(4-((6-(((1-fluorocyclopropyl)methoxy)methyl)-1,4-dioxan-2-yl)methoxy)phenyl)-2-oxo-6-(trifluoromethyl)-1,2-dihydropyridine-3-carboxamide;
5-(4-((6-((3,3-difluorocyclobutoxy)methyl)-1,4-dioxan-2-yl)methoxy)phenyl)-2-oxo-6-(trifluoromethyl)-1,2-dihydropyridine-3-carboxamide;
5-(4-((6-((2,2-difluoroethoxy)methyl)-1,4-dioxan-2-yl)methoxy)phenyl)-2-oxo-6-(trifluoromethyl)-1,2-dihydropyridine-3-carboxamide;
5-(4-((6-((2-methoxy-2-methylpropoxy)methyl)-1,4-dioxan-2-yl)methoxy)phenyl)-2-oxo-6-(trifluoromethyl)-1,2-dihydropyridine-3-carboxamide;
2-oxo-5-(4-((6-((trifluoromethoxy)methyl)-1,4-dioxan-2-yl)methoxy)phenyl)-6-(trifluoromethyl)-1,2-dihydropyridine-3-carboxamide;
5-(4-((6-((1H-pyrrol-1-yl)methyl)-1,4-dioxan-2-yl)methoxy)phenyl)-2-oxo-6-(trifluoromethyl)-1,2-dihydropyridine-3-carboxamide;
5-(4-((6-((1H-pyrazol-1-yl)methyl)-1,4-dioxan-2-yl)methoxy)phenyl)-2-oxo-6-(trifluoromethyl)-1,2-dihydropyridine-3-carboxamide;
5-(4-((6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide;
5-(4-((3-cyclopropyl-6,7-dihydro-4H-[1,2,3]triazolo[5,1-c][1,4]oxazin-6-yl)methoxy)phenyl)-2-oxo-6-(trifluoromethyl)-1,2-dihydropyridine-3-carboxamide;
5-(4-((3-cyclopropyl-6,7-dihydro-4H-[1,2,3]triazolo[5,1-c][1,4]oxazin-7-yl)methoxy)phenyl)-2-oxo-6-(trifluoromethyl)-1,2-dihydropyridine-3-carboxamide;
5-(4-((6-(2-methoxypropan-2-yl)-1,4-dioxan-2-yl)methoxy)phenyl)-2-oxo-6-(trifluoromethyl)-1,2-dihydropyridine-3-carboxamide;
5-(4-((6-(3-fluoropentan-3-yl)-1,4-dioxan-2-yl)methoxy)phenyl)-2-oxo-6-(trifluoromethyl)-1,2-dihydropyridine-3-carboxamide;
5-(4-((6-(hydroxymethyl)-1,4-dioxan-2-yl)methoxy)phenyl)-2-oxo-6-(trifluoromethyl)-1,2-dihydropyridine-3-carboxamide;
5-(4-((6-((3-fluoroazetidin-1-ylmethyl)-1,4-dioxan-2-yl)methoxy)phenyl)-2-oxo-6-(trifluoromethyl)-1,2-dihydropyridine-3-carboxamide;
5-(4-((6-((3,3-difluoroazetidin-1-yl)methyl)-1,4-dioxan-2-yl)methoxy)phenyl)-2-oxo-6-(trifluoromethyl)-1,2-dihydropyridine-3-carboxamide;
5-(4-((6-((3-methoxyazetidin-1-yl)methyl)-1,4-dioxan-2-yl)methoxy)phenyl)-2-oxo-6-(trifluoromethyl)-1,2-dihydropyridine-3-carboxamide;
5-(4-((2-fluoro-1,4-dioxan-2-yl)methoxy)phenyl)-2-oxo-6-(trifluoromethyl)-1,2-dihydropyridine-3-carboxamide;
5-(4-((1,4-dioxan-2-yl)difluoromethoxy)phenyl)-2-oxo-6-(trifluoromethyl)-1,2-dihydropyridine-3-carboxamide;
5-(4-(((1S,3R,6R)-7,7-difluoro-2,5-dioxabicyclo[4.1.0]heptan-3-yl)methoxy)phenyl)-2-oxo-6-(trifluoromethyl)-1,2-dihydropyridine-3-carboxamide;
aka: 5-(4-((cis-7,7-difluoro-2,5-dioxabicyclo[4.1.0]heptan-3-yl)methoxy)phenyl)-2-oxo-6-(trifluoromethyl)-1,2-dihydropyridine-3-carboxamide;
5-(4-(((1R,3R,6S)-7,7-difluoro-2,5-dioxabicyclo[4.1.0]heptan-3-yl)methoxy)phenyl)-2-oxo-6-(trifluoromethyl)-1,2-dihydropyridine-3-carboxamide;
aka: 5-(4-((trans-7,7-difluoro-2,5-dioxabicyclo[4.1.0]heptan-3-yl)methoxy)phenyl)-2-oxo-6-(trifluoromethyl)-1,2-dihydropyridine-3-carboxamide;
5-(4-((6-(methoxymethyl)-5-methyl-1,4-dioxan-2-yl)methoxy)phenyl)-2-oxo-6-(trifluoromethyl)-1,2-dihydropyridine-3-carboxamide;
5-(4-(((1S,3S,6R)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide;
5-(4-(((1R,3S,6S)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide;
5-(4-(((1R,3R,6S)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide;
5-(4-(((1S,3R,6R)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide;
5-(4-(((2S,6S)-6-(methoxymethyl)-1,4-dioxan-2-yl)methoxy)phenyl)-2-oxo-6-(trifluoromethyl)-1,2-dihydropyridine-3-carboxamide;
5-(4-(((2S,6R)-6-(methoxymethyl)-1,4-dioxan-2-yl)methoxy)phenyl)-2-oxo-6-(trifluoromethyl)-1,2-dihydropyridine-3-carboxamide;
5-(4-(((2R,6R)-6-(methoxymethyl)-1,4-dioxan-2-yl)methoxy)phenyl)-2-oxo-6-(trifluoromethyl)-1,2-dihydropyridine-3-carboxamide;
5-(4-(((2R,6S)-6-(methoxymethyl)-1,4-dioxan-2-yl)methoxy)phenyl)-2-oxo-6-(trifluoromethyl)-1,2-dihydropyridine-3-carboxamide;
5-(4-(((2S,6R)-6-(isopropoxymethyl)-1,4-dioxan-2-yl)methoxy)phenyl)-2-oxo-6-(trifluoromethyl)-1,2-dihydropyridine-3-carboxamide;
5-(4-(((2S,6S)-6-(isopropoxymethyl)-1,4-dioxan-2-yl)methoxy)phenyl)-2-oxo-6-(trifluoromethyl)-1,2-dihydropyridine-3-carboxamide;
5-(4-(((2S,6S)-6-cyclopropyl-1,4-dioxan-2-yl)methoxy)phenyl)-2-oxo-6-(trifluoromethyl)-1,2-dihydropyridine-3-carboxamide;
5-(4-(((2S,6S)-6-(methoxymethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-(methoxymethyl)-6-methyl-1,4-dioxan-2-yl)methoxy)phenyl)-2-oxo-6-(trifluoromethyl)-1,2-dihydropyridine-3-carboxamide;
5-(4-(((2R,6R)-6-(methoxymethyl)-6-methyl-1,4-dioxan-2-yl)methoxy)phenyl)-2-oxo-6-(trifluoromethyl)-1,2-dihydropyridine-3-carboxamide;
5-(4-(((2R,6S)-6-(methoxymethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-(dimethylcarbamoyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-((methoxy-d3)methyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-(cyanomethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-methyl-6-((methylsulfonyl)methyl)-1,4-dioxan-2-yl)methoxy)phenyl)-2-oxo-6-(trifluoromethyl)-1,2-dihydropyridine-3-carboxamide;
5-(4-(((2S,6R)-6-((cyclopropylsulfonyl)methyl)-6-methyl-1,4-dioxan-2-yl)methoxy)phenyl)-2-oxo-6-(trifluoromethyl-1,2-dihydropyridine-3-carboxamide;
(S)-5-(4-((6,6-bis(methoxymethyl)-1,4-dioxan-2-yl)methoxy)phenyl)-2-oxo-6-(trifluoromethyl)-1,2-dihydropyridine-3-carboxamide;
(S)-5-(4-((2,5,8-trioxaspiro[3.5]nonan-6-yl)methoxy)phenyl)-2-oxo-6-(trifluoromethyl)-1,2-dihydropyridine-3-carboxamide;
(S)-5-(4-((1,4,8,11-tetraoxaspiro[5.6]dodecan-2-yl)methoxy)phenyl)-2-oxo-6-(trifluoromethyl)-1,2-dihydropyridine-3-carboxamide;
5-(4-(((2S,6S)-6-(fluoromethyl)-6-(methoxymethyl)-1,4-dioxan-2-yl)methoxy)phenyl)-2-oxo-6-(trifluoromethyl)-1,2-dihydropyridine-3-carboxamide;
5-(4-(((2S,6S)-6-(chloromethyl)-6-(methoxymethyl)-1,4-dioxan-2-yl)methoxy)phenyl)-2-oxo-6-(trifluoromethyl)-1,2-dihydropyridine-3-carboxamide;
5-(4-(((2S,6S)-6-(fluoromethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-(fluoromethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((3-fluoroazetidin-1-yl)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((3,3-difluoroazetidin-1-yl)methyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-((dimethylamino)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((dimethylamino)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((difluoromethoxy)methyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-(2-methoxyethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-methyl-6-((oxetan-3-yloxy)methyl)-1,4-dioxan-2-yl)methoxy)phenyl)-2-oxo-6-(trifluoromethyl)-1,2-dihydropyridine-3-carboxamide;
5-(4-(((2S,6S)-6-(ethoxymethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-methyl-6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide;
5-(4-(((2S,6S)-6-((2-methoxyethoxy)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((2,2-difluoroethoxy)methyl)-6-methyl-1,4-dioxan-2-yl)methoxy)phenyl)-2-oxo-6-(trifluoromethyl-1,2-dihydropyridine-3-carboxamide;
5-(4-(((2S,6R)-6-methyl-6-((2,2,2-trifluoroethoxy)methyl)-1,4-dioxan-2-yl)methoxy)phenyl)-2-oxo-6-(trifluoromethyl)-1,2-dihydropyridine-3-carboxamide;
5-(4-(((2S,6S)-6-(hydroxymethyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-(2,2-difluoroethyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-(2-fluoroethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((2-fluoroethoxy)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-cyclopropyl-6-methyl-1,4-dioxan-2-yl)methoxy)phenyl)-2-oxo-6-(trifluoromethyl)-1,2-dihydropyridine-3-carboxamide;
5-(4-(((2S,6R)-6-cyclopropyl-6-(methoxymethyl)-1,4-dioxan-2-yl)methoxy)phenyl)-2-oxo-6-(trifluoromethyl)-1,2-dihydropyridine-3-carboxamide;
5-(4-(((2S,6S)-6-cyclopropyl-6-(methoxymethyl)-1,4-dioxan-2-yl)methoxy)phenyl)-2-oxo-6-(trifluoromethyl)-1,2-dihydropyridine-3-carboxamide,
and/or
5-(4-(((5R,7S)-2,6,9-trioxaspiro[4.5]decan-7-yl)methoxy)phenyl)-2-oxo-6-(trifluoromethyl)-1,2-dihydropyridine-3-carboxamide.

Embodiment 28. The compound of any one of Embodiments above, wherein R³ is L₁R⁵.

Embodiment 29. The compound of any one of Embodiments above, wherein R³ is L₂R⁹.

Embodiment 30. The compound of any one of Embodiments above, wherein R⁴ is halo, CN, C₁-C₈alkyl, C₁-C₈alkoxy or C₃-C₈cycloalkyl.

Embodiment 31. The compound of any one of Embodiments above, wherein R⁴ is halo.

Embodiment 32. The compound of any one of Embodiments above, wherein R⁴ is CN.

Embodiment 33. The compound of any one of Embodiments above, wherein R⁴ is C₁-C₈alkyl.

Embodiment 34. The compound of any one of Embodiments above, wherein R⁴ is C₁-C₈alkoxy.

Embodiment 35. The compound of any one of Embodiments above, wherein R⁴ is C₃-C₈cycloalkyl.

Embodiment 36. The compound of any one of Embodiments above, wherein L₁ is a bond, —CH₂—, —(CH₂)ₘ—, —OCH₂—, —O—, —CH₂O—, —O(CH₂)ₘ—, —CH₂OCH₂—, —CH(R⁷)—, —OCH(R⁷)—, —CH(R¹⁰)—, —OCH(R¹⁰)—, —CF₂—, —CF₂CH₂—, —OCF₂—, —OCH₂C(═O)—, —OCH₂C(═O)NH—, —OCH₂CH(OH)CH₂—, —CH₂OCH₂C(R⁷)₂—, —C(R⁷)₂—, —OC(R⁷)₂—, —OCH(R¹⁰)CH₂—, —NH— or —NH(CH₂)ₘ—.

Embodiment 37. The compound of any one of Embodiments above, wherein L₁ is —CH₂—, —OCH₂—, —O—, —CH₂O—, —O(CH₂)ₘ—, —CH(R⁷)—, —OCH(R⁷)—, —CH(R¹⁰)—, —OCH(R⁷)—, —CF₂CH₂—, —OCF₂—, —OCH₂C(═O)—, —OCH₂C(═O)NH—, or —OCH₂CH(OH)CH₂.

Embodiment 38. The compound of any one of Embodiments above, wherein L₁ is —CH₂—, —OCH₂—, —O—, or —CH₂O—.

Embodiment 39. The compound of any one of Embodiments above, wherein L₂ is a bond, —CH₂—, —(CH₂)ₘ—, —OCH₂—, —O—, —CH₂O—, —O(CH₂)ₘ—, —CH₂OCH₂—, —CH(R⁷)—, —OCH(R⁷)—, —CH(R¹⁰)—, —OCH(R¹⁰)—, —CF₂—, —CF₂CH₂—, —OCF₂—, —OCH₂C(═O)—, —OCH₂C(═O)NH—, —OCH₂CH(OH)CH₂—, —CH₂OCH₂C(R⁷)₂—, —C(R⁷)₂—, —OC(R⁷)₂—, —OCH(R⁷)CH₂—, —NH— or —NH(CH₂)ₘ—.

Embodiment 40. The compound of any one of Embodiments above, wherein L₂ is bond, —CH₂—, —OCH₂—, —O(CH₂)ₘ—, —CH(R⁷)—, —OCH(R¹⁰)—, —OCH(R¹⁰)CH₂— or —OCH₂C(═O)—.

Embodiment 41. The compound of any one of Embodiments above, wherein L₂ is bond, —CH₂— or —OC_{H}r.

Embodiment 42. The compound of any one of Embodiments above, wherein L₃ is a bond, —CH₂—, —(CH₂)ₘ—, —OCH₂—, —O—, —CH₂O—, —O(CH₂)ₘ—, —CH₂OCH₂—, —CH(R⁷)—, —OCH(R⁷)—, —CH(R¹⁰)—, —OCH(R¹⁰)—, —CF₂—, —CF₂CH₂—, —OCF₂—, —OCH₂C(═O)—, —OCH₂C(═O)NH—, —OCH₂CH(OH)CH₂—, —CH₂OCH₂C(R⁷)₂—, —C(R⁷)₂—, —OC(R⁷)₂—, —OCH(R¹⁰)CH₂—, —NH— or —NH(CH₂)ₘ—.

Embodiment 43. The compound of any one of Embodiments above, wherein L₃ is a bond, —CH₂—, —OCH₂—, —O—, —NH—, —NH(CH₂)ₘ—, —CH₂OCH₂— or —CH₂O—.

Embodiment 44. The compound of any one of Embodiments above, wherein L₃ is a bond, —CH₂—, —OCH₂—, —CH₂OCHᵣ or —CH₂O—.

Embodiment 45. The compound of any one of Embodiments above, wherein L₄ is a bond, —CH₂—, —(CH₂)ₘ—, —OCH₂—, —O—, —CH₂O—, —O(CH₂)ₘ—, —CH₂OCH₂—, —CH(R⁷)—, —OCH(R⁷)—, —CH(R¹⁰)—, —OCH(R¹⁰)—, —CF₂—, —CF₂CH₂—, —OCF₂—, —OCH₂C(═O)—, —OCH₂C(═O)NH—, —OCH₂CH(OH)CH₂—, —CH₂OCH₂C(R⁷)₂—, —C(R⁷)₂—, —OC(R⁷)₂—, —OCH(R¹⁰)CH₂—, —NH— or —NH(CH₂)ₘ—.

Embodiment 46. The compound of any one of Embodiments above, wherein L₄ is a —CH₂—, —(CH₂)ₘ—, —C(R⁷)₂—, —CF₂—, —OCH₂—, —O(CH₂)ₘ—, —CH₂O—, —CH₂OCH₂—, —CH₂OCH₂C(R⁷)₂— or —NH(CH₂)ₘ—.

Embodiment 47. The compound of any one of Embodiments above, wherein L₄ is a bond, —CH₂—, —(CH₂)ₘ—, —C(R⁷)₂—, —CF₂—, —OCH₂—, —O(CH₂)ₘ—, —CH₂O—, —CH₂OCH₂—, or —CH₂OCH₂C(R⁷)₂—.

Embodiment 48. The compound of any one of Embodiments above, wherein L₄ is a bond, —CH₂—, —(CH₂)₂—, —C(CH₃)₂—, —C(CH₂CH₃)₂—, —CF₂—, —CH₂O—, —CH₂OCH₂—, —CH₂OCH₂C(CH₃)₂— or —CH₂OCH₂CH₂.

Embodiment 49. The compound of any one of Embodiments above, wherein L₄ is —CH₂—, or —CH₂O—.

Embodiment 50. The compound of any one of Embodiments above, wherein R⁵ is phenyl substituted with 0-3 groups independently selected from R⁹.

Embodiment 51. The compound of any one of Embodiments above, wherein R⁵ is C₃-C₈cycloalkyl substituted with 0-3 groups independently selected from R⁹;

Embodiment 52. The compound of any one of Embodiments above, wherein R⁵ is a 4-8 membered monocyclic heterocycloalkyl group having 1 to 3 ring members independently selected from N, NR⁷, O, S, C═O or S(═O)₂, substituted with 0-3 groups independently selected from R⁹.

Embodiment 53. The compound of any one of Embodiments above, wherein R⁵ is a 9-12 membered heterocyclyl group having 1 to 4 ring members independently selected from N, NR⁷, O, or S, substituted with 0-3 groups independently selected from R⁹.

Embodiment 54. The compound of any one of Embodiments above, wherein R⁵ is a 7-8 membered bridged-bicyclic heterocycloalkyl group having 1 to 2 ring members independently selected from N, NR⁷, O, S, C═O or S(═O)₂, substituted with 0-3 groups independently selected from R⁹.

Embodiment 55. The compound of any one of Embodiments above, wherein R⁵ is a 6-8 membered fused bicyclic heterocycloalkyl group having 1 to 2 ring members independently selected from N, NR⁷, O, S, C═O or S(═O)₂, substituted with 0-3 groups independently selected from R⁹.

Embodiment 56. The compound of any one of Embodiments above, wherein R⁵ is a 5-8 membered heteroaryl having 1 to 3 ring members independently selected from N, NR⁷, 0 or S, substituted with 0-3 groups independently selected from R⁹.

Embodiment 57. The compound of any one of Embodiments above, wherein R⁵ is a 9-10 membered heteroaryl having 1 to 4 ring members independently selected from N, NR⁷, N⁺O⁻, O, or S, substituted with 0-3 groups independently selected from R⁹.

Embodiment 58. The compound of any one of Embodiments above, wherein R⁶ is —CN, —OH, —NR⁷R⁸, —NR⁷C(═O)R⁸, —NR⁷S(═O)₂R⁸, —C(═O)NR⁷R⁸, —S(═O)₂R⁸, halo, C₁-C₈haloalkoxy, C₁-C₈-alkoxy, —C₁-C₈alkyl or —C₁-C₈alkylene.

Embodiment 59. The compound of any one of Embodiments above, wherein R⁹ is —C₁-C₈alkyl.

Embodiment 60. The compound of any one of Embodiments above, wherein each R⁹ is independently selected from —C₁-C₈alkyl, —C₁-C₈-alkoxy, C₁-C₈haloalkyl, spiro attached C₃-C₈cycloalkyl, R¹⁰, —OH, CN, halo, —C(═O)R⁷, —NR⁷R⁸, —C(═O)NR⁷R⁸, —NR⁷C(═O)R⁸, —NR⁷C(═O)OR⁸, —C(═O)OR⁸, —NR⁷S(═O)₂R⁸, —S(═O)₂R⁸, —S(═O)R¹, NO₂, CN, L₃R¹¹, L₄R¹² or a spiro attached 4-8 membered monocyclic heterocycloalkyl group having 1 to 2 ring members independently selected from N, NR⁷, O, or S.

Embodiment 61. The compound of any one of Embodiments above, wherein each R⁹ is independently selected from —C₁-C₈alkyl, —C₁-C₈-alkoxy, C₁-C₈haloalkyl, spiro attached C₃-C₈cycloalkyl, R¹⁰, —OH, CN, halo, L₄R¹² or a spiro attached 4-8 membered monocyclic heterocycloalkyl group having 1 to 2 ring members independently selected from N, NR⁹, O, or S.

Embodiment 62. The compound of any one of Embodiments above, wherein each R⁹ is independently selected from —R¹⁰ or L₄R¹².

Embodiment 63. The compound of any one of Embodiments above, wherein R¹⁰ is a C₃-C₈cycloalkyl, a 4-8 membered monocyclic heterocycloalkyl group having 1 to 2 ring members independently selected from N, NR⁷, O or S, or a 5-6 membered heteroaryl having 1 to 2 ring members independently selected from N, NR⁹, O, or S.

Embodiment 64. The compound of any one of Embodiments above, wherein R¹⁰ is a C₃-C₈cycloalkyl.

Embodiment 65. The compound of any one of Embodiments above, wherein R¹⁰ is a 4-8 membered monocyclic heterocycloalkyl group having 1 to 2 ring members independently selected from N, NR⁹, O or S.

Embodiment 66. The compound of any one of Embodiments above, wherein R¹⁰ is a 5-6 membered heteroaryl having 1 to 2 ring members independently selected from N, NR⁷, O, or S.

Embodiment 67. The compound of any one of Embodiments above, wherein R¹¹ is phenyl substituted with 0-3 groups independently selected from R¹³.

Embodiment 68. The compound of any one of Embodiments above, wherein R¹¹ is C₃-C₈cycloalkyl substituted with 0-3 groups independently selected from R¹³.

Embodiment 69. The compound of any one of Embodiments above, wherein R¹¹ is a 4-8 membered monocyclic heterocycloalkyl group having 1 to 3 ring members independently selected from N, NR⁷, O, S, C═O or S(═O)₂ substituted with 0-3 groups independently selected from R¹³.

Embodiment 70. The compound of any one of Embodiments above, wherein R¹¹ is a 9-12 membered heterocyclyl having 1 to 4 ring members independently selected from N, NR⁷, O, or S substituted with 0-3 groups independently selected from R¹³.

Embodiment 71. The compound of any one of Embodiments above, wherein R¹¹ is a 7-8 membered bridged-bicyclic heterocycloalkyl group having 1 to 2 ring members independently selected from N, NR⁷, O, S, C═O or S(═O)₂, substituted with 0-3 groups independently selected from R¹.

Embodiment 72. The compound of any one of Embodiments above, wherein R¹¹ is a 6-8 membered fused bicyclic heterocycloalkyl group having 1 to 2 ring members independently selected from N, NR⁷, O, S, C═O or S(═O)₂, substituted with 0-3 groups independently selected from R¹³;

Embodiment 73. The compound of any one of Embodiments above, wherein R¹¹ is a 5-6 membered heteroaryl having 1 to 3 ring members independently selected from N, NR⁷, O or S, substituted with 0-3 groups independently selected from R¹³

Embodiment 74. The compound of any one of Embodiments above, wherein R¹¹ is a 9-10 membered heteroaryl having 1 to 4 ring members independently selected from N, NR⁷, N⁺O⁻, O, or S substituted with 0-3 groups independently selected from R¹³;

Embodiment 75. The compound of any one of Embodiments above, wherein each R¹² is independently selected from —CN, —CD₃, —OCD₃, —OH, —NR⁷R⁸, —NR⁷C(═O)R⁸, —NR⁷S(═O)₂R⁸, —C(═O)NR⁷R⁸, —S(═O)₂R⁸, —S(═O)₂R¹⁰, halo, C₁-C₈haloalkoxy, C₁-C₈-alkoxy, C₁-C₈haloalkyl or —C₁-C₈alkyl.

Embodiment 76. The compound of any one of Embodiments above, wherein each R¹² is independently selected from —CN, —OH, —NR⁷R⁸, —NR⁷C(═O)R⁸, —NR⁷S(═O)₂R⁸, —C(═O)NR⁷R⁸, —S(═O)₂R⁸, —S(═O)₂R¹⁰, halo, C₁-C₈haloalkoxy, C₁-C₈-alkoxy, C₁-C₈haloalkyl or —C₁-C₈alkyl.

Embodiment 77. The compound of any one of Embodiments above, wherein each R¹³ is independently selected from —C₁-C₈alkyl, spiro attached C₃-C₈cycloalkyl, —C₁-C₈-alkoxy, —OH, halo, —C(═O)R⁷, —NR⁷R⁸, —C(═O)NR⁷R⁸, —NR⁷C(═O)R⁸, —NR⁷S(═O)₂R⁸, —S(═O)₂R⁸, NO₂, CN or C₁-C₈haloalkyl.

Embodiment 78. The compound of any one of Embodiments above, wherein each R¹³ is independently selected from —C₁-C₈alkyl, —C₁-C₈-alkoxy, —OH or halo.

Embodiment 79. The compound of any one of Embodiments above, wherein each m is independently selected from 1, 2, 3 or 4.

Embodiment 80. The compound of any one of Embodiments above, wherein each n is independently selected from 0, 1, 2 or 3.

Embodiment 81. The compound of any one of Embodiments above, wherein each p is independently selected from 0, 1, 2 or 3.

Embodiment 82. The compound of any one of Embodiments above, wherein m is independently selected from 1, 2 or 3.

Embodiment 83. The compound of any one of Embodiments above, wherein each n is 0.

Embodiment 84. The compound of any one of Embodiments above, wherein each p is independently selected from 0 and 1.

Embodiment 85. The compound of any one of Embodiments above, wherein each R⁷ is independently selected from H or —C₁-C₈alkyl.

Embodiment 86. The compound of any one of Embodiments above, wherein each R⁷ is H.

Embodiment 87. The compound of any one of Embodiments above, wherein each R⁷ is —C₁-C₈alkyl.

Embodiment 88. The compound of any one of Embodiments above, wherein R⁸ is H or —C₁-C₈alkyl.

Embodiment 89. The compound of any one of Embodiments above, wherein R⁸ is H.

### General Synthetic Procedures

The provided compounds may be produced by organic synthesis methods known to one of ordinary skill in the art with reference to the following reaction general synthetic schemes below and in more detail in the Examples.

All starting materials, building blocks, reagents, acids, bases, dehydrating agents, solvents, and catalysts utilized to synthesize the provided compounds are either commercially available or may be produced by organic synthesis methods known to one of ordinary skill in the art (Houben-Weyl 4th Ed. 1952, Methods of Organic Synthesis, Thieme, Volume 21).

Within the scope of this text, only a readily removable group that is not a constituent of the particular desired end product of the provided compounds is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005.41627 pp. (URL: http-/www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der Organischen Chemie” (Methods of Organic Chemistry), Houben Weyl, 4th edition, Volume 15/1, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they may be removed readily (i.e., without the occurrence of undesired secondary reactions) for example by solvolysis, reduction, photolysis or alternatively under physiological conditions (e.g., by enzymatic cleavage).

The disclosure further provides processes to make the compounds of Formula (I) as disclosed herein, and any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Provided compounds and intermediates may also be converted into each other according to methods generally known to those skilled in the art.

Methods to synthesize compounds of Formula (I) are depicted in Schemes A-G and are illustrated by the Examples herein. Scheme A depicts a way to prepare compounds wherein R can be a variety of groups and linkages. It begins with by forming a 2-oxo-6-(trifluoromethyl)-1,2-dihydropyridine-3-carboxylate compound, where the 3-carboxylate is protected with a suitably removable protecting group which can be converted to an amide and the 5-position of the ring has a suitable leaving group that can readily be condensed with a suitably substituted phenyl borate to provide either the provided compounds or a compound with a suitable linkage for further manipulation as depicted in Schemes B-G.

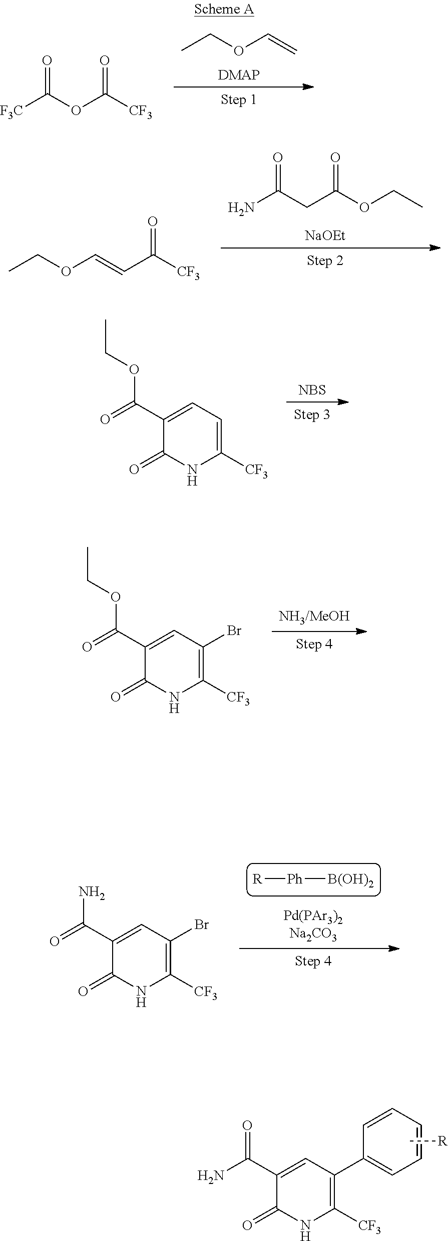

Accordingly, schemes B-G depict methods to make compounds of Formula (I) wherein R is variously substituted aminomethyl group, a substituted methoxy group and more specifically a racemic or chiral substituted-1,4-dioxan-2-yl)methoxy group, respectively.

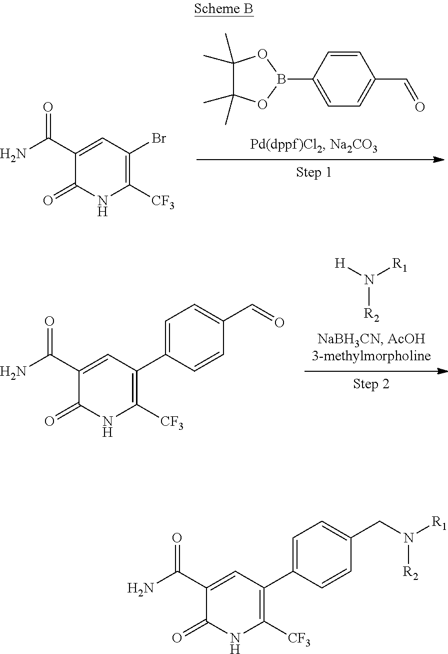

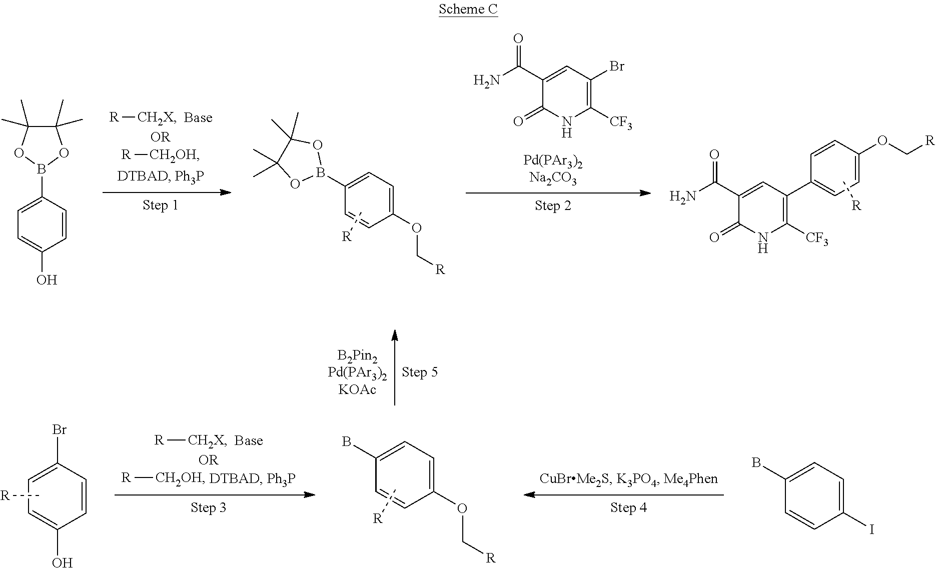

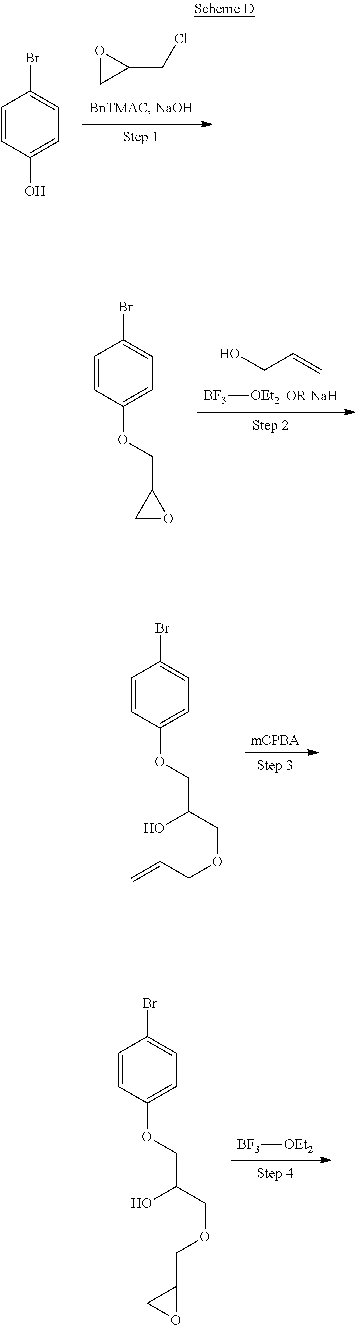

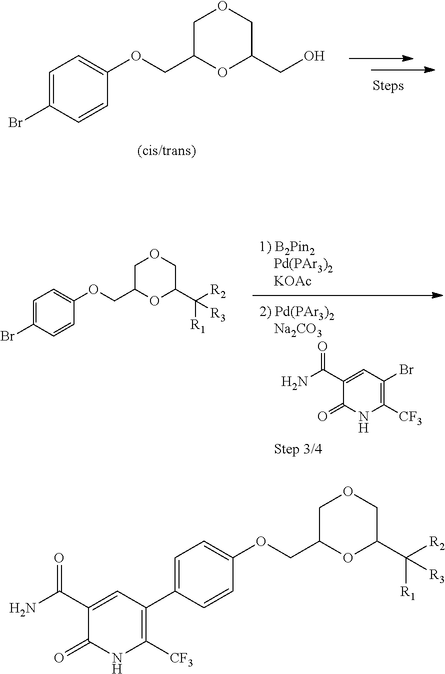

Scheme D depicts methods to make compounds of Formula (I) having a substituted-1,4-dioxan-2-yl)methoxy group.

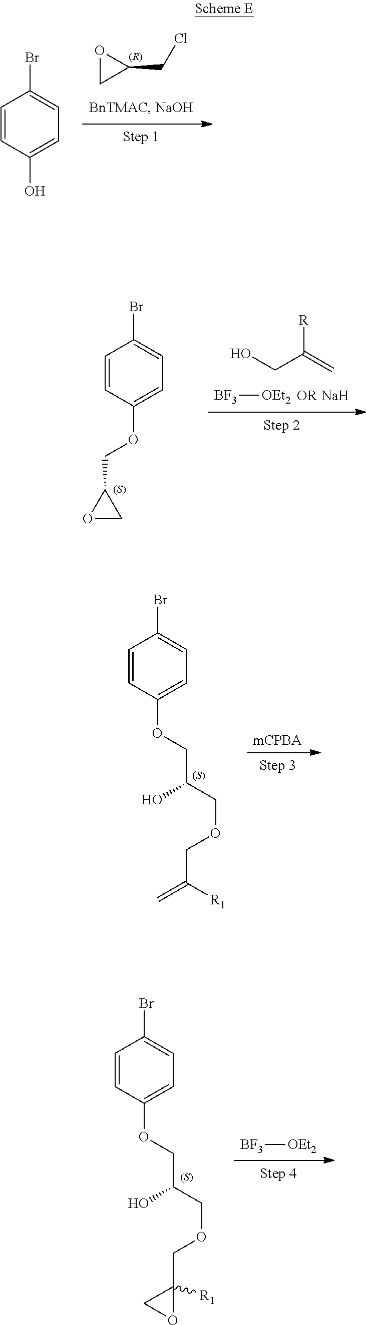

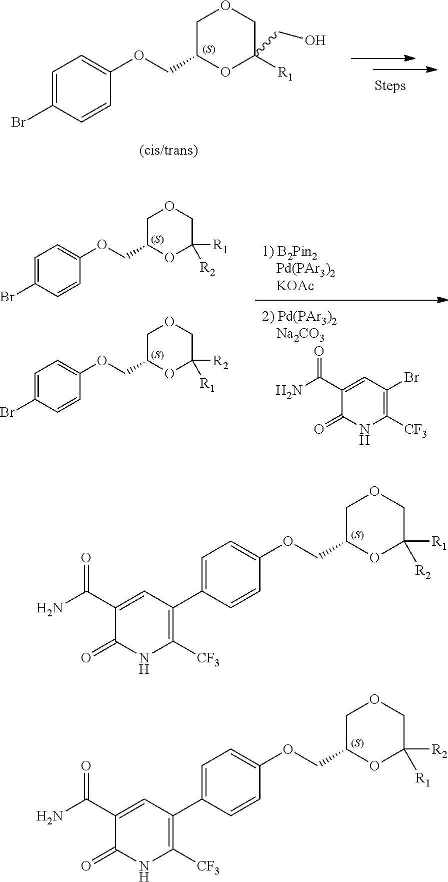

Scheme E depict methods to make compounds of Formula (I) having a chiral substituted-1,4-dioxan-2-yl))methoxy group.

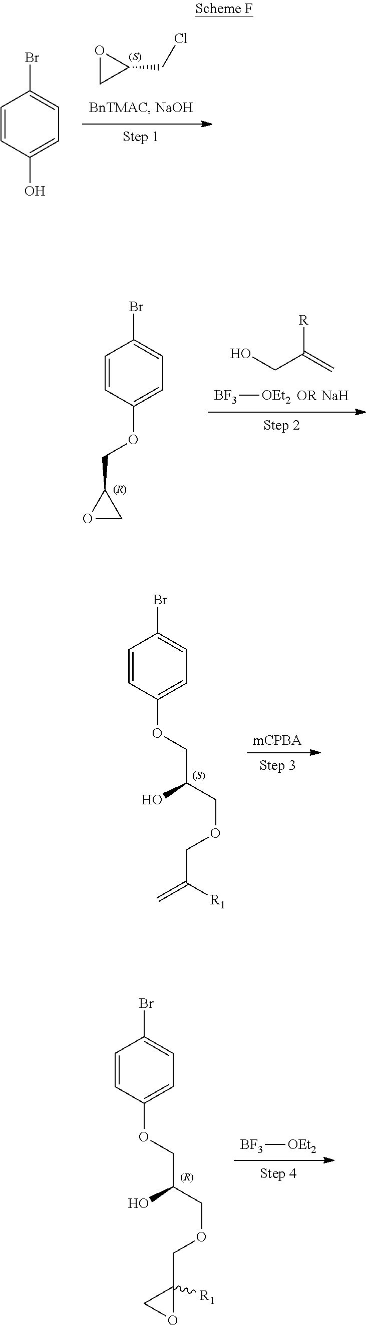

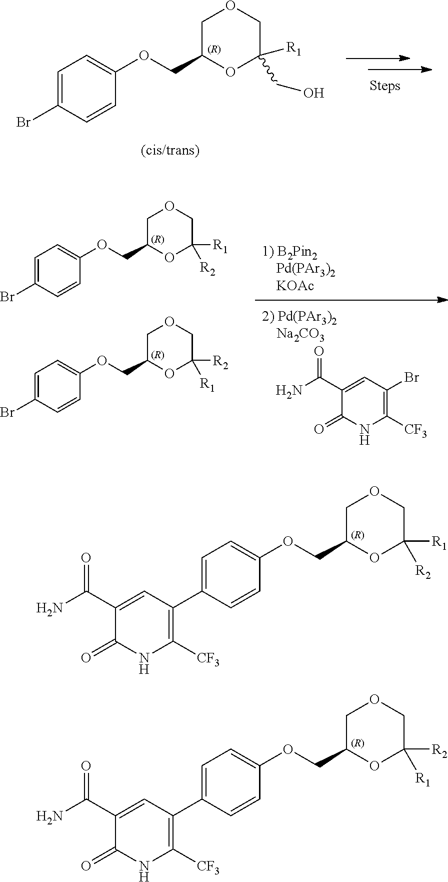

Scheme F depicts methods to make compounds of Formula (I) having a chiral substituted-1,4-dioxan-2-yl))methoxy group

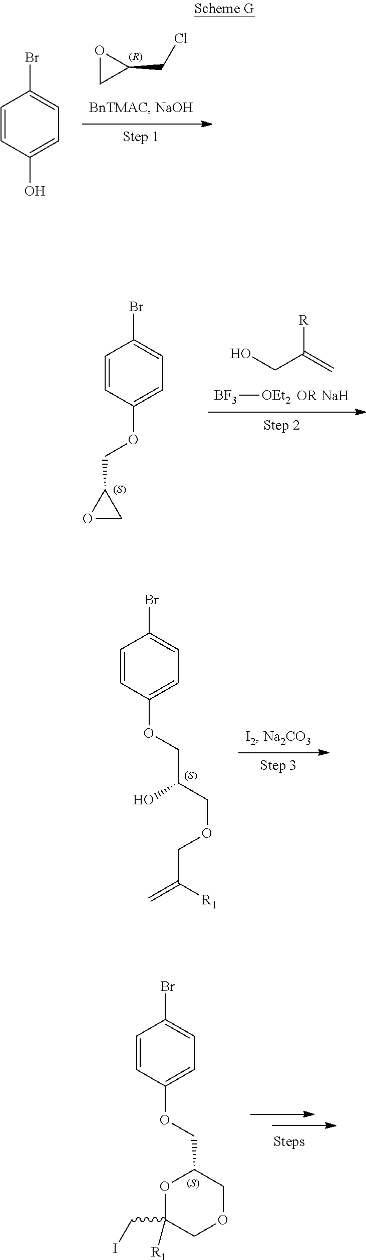

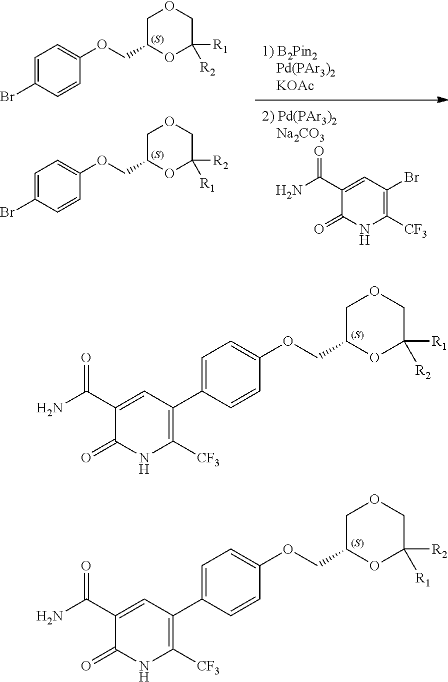

Scheme G depicts methods to make compounds of Formula (I) wherein the compounds have a chiral substituted-1,4-dioxan-2-yl)methoxy group.

Using these synthesis schemes and the examples provided, the skilled person can prepare the compounds of Formula (I).

Depending on the choice of the starting materials and synthesis procedures, the compounds may be present in the form of one of the possible isomers or as mixtures thereof, for example as pure optical isomers, or as isomer mixtures, such as racemates and diastereoisomer mixtures, depending on the number of asymmetric carbon atoms. The present disclosure includes all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration unless specified. If the compound contains a di-substituted cycloalkyl, the cycloalkyl substituent may have a cis- or trans-configuration, unless otherwise specified. All tautomeric forms are also intended to be included.

Any asymmetric atom (e.g., carbon or the like) of the provided compound(s) may be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess of either the (R)- or (S)-configuration; i.e., for optically active compounds, it is often preferred to use one enantiomer to the substantial exclusion of the other enantiomer, so typically an enantiomeric purity of at least 95% is preferred. Substituents at atoms with unsaturated double bonds may, if possible, be present in cis-(Z)- or trans-(E)-form.

Accordingly, as used herein a provided compound may be in the form of one of the possible isomers, rotamers, atropisomers, tautomers or mixtures thereof, for example, as substantially pure geometric (cis or trans) isomers, diastereomers, optical isomers (antipodes), racemates or mixtures thereof. ‘Substantially pure’ or ‘substantially free of other isomers’ as used herein means the product contains less than 5%, and preferably less than 2%, of other isomers relative to the amount of the preferred isomer, by weight.

Resulting mixtures of isomers may typically be separated on the basis of the physicochemical differences of the constituents, into the pure or substantially pure geometric or optical isomers, diastereomers, racemates, for example, by chromatography and/or fractional crystallization.

Racemates of final products or intermediates may typically be resolved into the optical antipodes by known methods, e.g., by separation of the diastereomeric salts thereof, obtained with an optically active acid or base, and liberating the optically active acidic or basic compound. In particular, a basic moiety may thus be employed to resolve the provided compounds into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products may also be resolved by chiral chromatography, e.g., high pressure liquid chromatography (HPLC) using a chiral stationary phase.

In many cases, the provided compounds are capable of forming acid and/or base salts by virtue of the presence of amino and/or carboxyl groups or groups similar thereto. As used herein, the terms “salt” or“salts” refers to an acid addition or base addition salt of a provided compound. “Salts” include in particular “pharmaceutical acceptable salts”. The term “pharmaceutically acceptable salts” refers to salts that retain the biological effectiveness and properties of the provided compounds and which typically are not biologically or otherwise undesirable.

Pharmaceutically acceptable acid addition salts may be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlorotheophyllinate, citrate, ethanedisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate or trifluoroacetate salts. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Inorganic acids from which salts may be derived include, for example, hydrochloric acid, hydrobromic acid, sulfuric acid, nitric acid, phosphoric acid, or the like.

Organic acids from which salts may be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, or the like. Pharmaceutically acceptable base addition salts may be formed with inorganic or organic bases and may have inorganic or organic counterions.

Inorganic counterions for such base salts include, for example, ammonium salts and metals from columns I to XII of the periodic table. In certain embodiments, the counterion is selected from sodium, potassium, ammonium, alkylammonium having one to four C1-C4 alkyl groups, calcium, magnesium, iron, silver, zinc, or copper; particularly suitable salts include ammonium, potassium, sodium, calcium or magnesium salts.

Organic bases from which salts may be derived include, for example, primary, secondary, or tertiary amines, substituted amines including naturally occurring substituted amines, cyclic amines, basic ion exchange resins, and the like. Suitable organic amines include isopropylamine, benzathine, cholinate, diethanolamine, diethylamine, lysine, meglumine, piperazine or tromethamine.

The pharmaceutically acceptable salts of the present disclosure may be synthesized from a basic or acidic moiety, by conventional chemical methods. Generally, such salts may be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, tetrahydrofuran, toluene, chloroform, dichloromethane, methanol, ethanol, isopropanol, or acetonitrile is desirable, where practicable.

Any formula given herein is also intended to represent unlabeled forms (i.e., compounds wherein all atoms are present at natural isotopic abundances, and not isotopically enriched) as well as isotopically enriched or labeled forms of the compounds. Isotopically enriched or labeled compounds have structures depicted by the formulas given herein except that at least one atom of the compound is replaced by an atom having an atomic mass or mass number different from the atomic mass or the atomic mass distribution that occurs naturally. Examples of isotopes that may be incorporated into enriched or labeled compounds of the disclosure include isotopes of hydrogen, carbon, nitrogen, oxygen, phosphorous, fluorine, or chlorine, such as ²H, ³H, ¹¹C, ¹³C, ¹⁴C, ¹⁵N, ¹⁸F, ³¹P, ³²P, ³⁵S, ³⁶Cl, or ¹²⁵I. The disclosure includes various isotopically labeled compounds as defined herein, for example those in which radioactive isotopes, such as ³H and ¹⁴C, or those in which non-radioactive isotopes, such as ²H and ¹³C, are present at levels significantly above the natural abundance for these isotopes. These isotopically labeled compounds are useful in metabolic studies (e.g., with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically-labeled compounds of formula (I) may generally be prepared by conventional techniques known to those skilled in the art or by processes analogous to those described in the accompanying Examples using an appropriate isotopically-labeled reagent in place of the non-labeled reagent otherwise employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a provided compound is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Furthermore, the provided compounds, including their salts, may also be obtained in the form of their hydrates, or include other solvents used for their crystallization. The provided compounds may inherently or by design form solvates with pharmaceutically acceptable solvents (including water); therefore, the disclosure embraces both solvated and unsolvated forms. The term “solvate” refers to a molecular complex of a provided compound (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Pharmaceutically acceptable solvates in accordance with the disclosure include those wherein the solvent of crystallization may be isotopically substituted, e.g. D₂O, d⁶-acetone, d⁶-DMSO, as well as solvates with non-enriched solvents.

The compounds disclosed herein, i.e., compounds of formula (I) that contain groups capable of acting as donors and/or acceptors for hydrogen bonds, may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO2004/078163. Hence the disclosure further provides co-crystals comprising a compound of formula (I).

The disclosure further provides additional compounds that, upon conversion within the body a subject, yield any of the compounds discussed above. These additional compounds are prodrug forms of the compounds discussed above. As used herein, the term “prodrug” refers to a precursor compound that, following administration to a subject, releases the biologically active compound in vivo via some chemical or physiological process (e.g., a prodrug on reaching physiological pH or through enzyme action is converted to the biologically active compound). A prodrug itself may either lack or possess the desired biological activity.

### Pharmaceutical Compositions and Routes of Administration

In another aspect, the present disclosure provides a pharmaceutical composition comprising a provided compound, or a pharmaceutically acceptable salt thereof, and at least one pharmaceutically acceptable carrier. In some embodiments, the pharmaceutical composition comprises at least two pharmaceutically acceptable excipients or carriers. Pharmaceutically acceptable carriers and other excipients are known to those of skill in the art, and may be selected, for example, from carriers and excipients used in approved (registered) formulated therapeutic agents that are administered via similar routes of administration. The pharmaceutical composition may be formulated for particular routes of administration such as oral administration, parenteral administration, rectal administration, or the like. In addition, the provided pharmaceutical compositions may be made up in a solid form (including without limitation capsules, tablets, pills, granules, powders or suppositories), or in a liquid form (including without limitation solutions, suspensions or emulsions). The pharmaceutical compositions may be subjected to conventional pharmaceutical operations such as sterilization and/or may contain conventional inert diluents, lubricating agents, or buffering agents, as well as adjuvants, such as preservatives, stabilizers, wetting agents, emulsifiers and/or buffers, etc.

In one embodiment, the provided compounds are formulated for oral delivery. Typically, these pharmaceutical compositions are tablets or gelatin capsules comprising the active ingredient (at least one compound of Formula (I)) together with one or more excipients selected from:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and/or sweeteners.

Tablets may be either film coated or enteric coated according to methods known in the art.

Suitable compositions for oral administration include an effective amount of a provided compound in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions may contain one or more agents selected from sweetening agents, flavoring agents, coloring agents and/or preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and/or lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate may be employed. Formulations for oral use may be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a provided compound with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems may pertain to an inhalation or to an intranasal application that may be suitable for use to treat influenza, for example, and may contain solubilizers, stabilizers, tonicity enhancing agents, buffers and/or preservatives. They may be conveniently delivered in the form of a dry powder (either alone, as a mixture, for example a dry blend with lactose, or a mixed component particle, for example with phospholipids) from a dry powder inhaler or an aerosol spray presentation from a pressurized container, pump, spray, atomizer or nebulizer, with or without the use of a suitable propellant.

The present disclosure further provides anhydrous pharmaceutical compositions and dosage forms comprising the provided compounds as active ingredients, since water may facilitate the degradation of certain compounds.

Anhydrous pharmaceutical compositions and dosage forms disclosed herein may be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they may be included in suitable formulary kits. Examples of suitable packaging include, but are not limited to, hermetically sealed foils, plastics, unit dose containers (e. g., vials), blister packs, and/or strip packs.

The disclosure further provides pharmaceutical compositions and dosage forms that comprise one or more agents that reduce the rate by which the provided compound as an active ingredient will decompose. Such agents, which are referred to herein as “stabilizers,” include, but are not limited to, antioxidants such as ascorbic acid, pH buffers, and/or salt buffers, etc.

The provided pharmaceutical composition or combination may be in unit dosage containing about 1-1000 mg of active ingredient(s) for a human subject of about 50-70 kg, or about 1-500 mg, or about 1-250 mg, or about 1-150 mg, or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in in vitro and in vivo tests using advantageously mammals, e.g., mice, rats, dogs, monkeys or isolated organs, tissues and preparations thereof. The provided compounds may be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations.

### Pharmacology and Utility

The compounds of formula (I), in free form or in salt form, exhibit valuable pharmacological properties, e.g. they inhibit or prevent replication of orthomyxovirus, as indicated by test data provided in the next sections, and are therefore indicated for therapy or for use as research chemicals, e.g. as tool compounds such as for the study of replication of an orthomyxovirus, particularly Influenza A, Influenza B or Influenza C. Accordingly, compounds disclosed herein are useful in the treatment of an infection caused by an orthymyxovirus, particularly Influenza A, Influenza B or Influenza C, especially in human subjects. In some embodiments, the subject to be treated is a human having or at risk of contracting an influenza viral infection, particularly Influenza A, Influenza B or Influenza C. For example, subjects having pre-existing conditions such as asthma or COPD that may be greatly exacerbated by an influenza infection may be treated with the provided methods or compounds before exhibiting symptoms of an influenza infection, particularly Influenza A, Influenza B or Influenza C, especially if they are at risk of contracting influenza due to close proximity to persons such as family members who have or appear to have influenza. In other embodiments, the subject for treatment by the provided methods and compositions is one diagnosed as having symptoms consistent with an influenza infection, particularly Influenza A, Influenza B or Influenza C. In other embodiments, the subject may be a human who has been tested with known diagnostic methods such as a Rapid Influenza Diagnostic Test (RIDT) or Reverse Transcriptase PCT (RT-PCR) methods to detect the presence of influenza virus, and found to be infected with influenza, regardless of the presence of typical influenza symptoms.

All methods described herein may be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the embodiments otherwise claimed.

In another aspect, the disclosure provides a method to treat a subject infected with influenza A, B, or C, which comprises administering to a subject in need of such treatment an effective amount of a compound of Formula (I) or any subgenus or species thereof as described herein, or a pharmaceutical composition comprising such compound or composition. The subject may be a mammal, and is preferably a human, although the provided compounds and methods are suitable for treatment of other species that contract Influenza A, Influenza B, or influenza C, or other orthomyxoviruses. The disclosure includes compounds of Formula (I) and the subgenera of Formula (I) described herein, and includes all stereoisomers (including diastereoisomers and enantiomers) except where a specific isomer is expressly described, as well as tautomers and isotopically enriched versions thereof (including deuterium substitutions) as well as pharmaceutically acceptable salts of these compounds.

As a further embodiment, the present disclosure provides the use of a compound of formula (I) or any of the embodiments within the scope of Formula (I) as described herein, in therapy. In particular, the compounds are suitable for use to treat a subject having or at particularly high risk for an orthomyxovirus viral infection, especially Influenza A, Influenza B, or Influenza C.

In another embodiment, the disclosure provides a method of treating a disease which is caused by an orthomyxovirus, comprising administration of a therapeutically effective amount of a compound of formula (I) or any of the embodiments within the scope of Formula (I) as described herein to a subject in need of such treatment. In some embodiments, the compound of formula (I) is administered orally. In a further embodiment, the disease is selected from Influenza A, Influenza B, and Influenza C. The method typically comprises administering an effective amount of a compound as described herein, or a pharmaceutical composition comprising an effective amount of such compound, to a subject in need of such treatment. The compound may be administered by any suitable method such as those described herein, and the administration may be repeated at intervals which may be selected by a treating physician. In some embodiments, the compound or pharmaceutical composition is administered orally.

Thus, as a further embodiment, the present disclosure provides the use of a compound of formula (I) or any of the embodiments of such compounds described herein for the manufacture of a medicament. In a particular embodiment, the medicament is for treatment of an orthomyxovirus infection, especially Influenza A, Influenza B, or Influenza C.

A further embodiment of the present disclosure provides the use of a compound of formula (I) or any of the embodiments of such compounds described herein for the manufacture of a medicament for the treatingment of influenza. A further embodiment of the present disclosure provides the use of a compound of formula (I) or any of the embodiments of such compounds described herein for the manufacture of a medicament for the treatment of Influenza A, Influenza B, or Influenza C.

In addition, the disclosure provides the use of a compound of formula (I) for treating a viral infection caused by an orthomyxovirus, particularly influenza, which may be Influenza A, Influenza B or Influenza C. In a further embodiment the disclosure provides the use of a compound of formula (I) for treating influenza. In a further embodiment the disclosure provides the use of a compound of formula (I) for treating Influenza A, Influenza B, or Influenza C.

### Combination Treatment

The compound disclosed herein may be administered either simultaneously with, or before or after, one or more therapeutic co-agent(s). The provided compound may be administered separately, by the same or different route of administration, or together in the same pharmaceutical composition as the co-agent(s). Suitable co-agents for use with the provided compounds include antivirals active on influenza viruses, such as neuraminidase inhibitors including oseltamivir, peramivir, zanamivir and laninamivir, laninamivir octanoate, and adamantanes such as amantadine and rimantadine. Additional co-agents for use in these methods include an M2 protein inhibitor, a polymerase inhibitor, a PB2 inhibitor, favipiravir, fludase, ADS-8902, beraprost, Neugene®, ribavirin, CAS Reg. No. 1422050-75-6, VX-787, Flu Mist Quadrivalent®, Fluarix® Quadrivalent, Fluzone® Quadrivalent, Flucelvax® or FluBlok®.

In one embodiment, the disclosure provides a product comprising a compound of formula (I) and at least one other therapeutic co-agent as a combined preparation for simultaneous, separate or sequential use in therapy. In one embodiment, the therapy is the treatment of a viral infection caused by an orthomyxovirus, particularly Influenza A, Influenza B or Influenza C. Products provided as a combined preparation include a composition comprising a compound of formula (I) and at least one of the other therapeutic co-agent(s) together in the same pharmaceutical composition, or the compound of formula (I) and at least one other therapeutic co-agent(s) in separate form, e.g. in the form of a kit for use to treat a subject by the methods described herein.

In one embodiment, the disclosure provides a pharmaceutical composition comprising a compound of formula (I) and another therapeutic co-agent(s). Suitable co-agents include antivirals active on influenza viruses, such as neuraminidase inhibitors including oseltamivir, peramivir, zanamivir and laninamivir, and adamantanes such as amantadine and rimantadine. Optionally, the pharmaceutical composition may comprise a pharmaceutically acceptable carrier, as described above.

In one embodiment, the disclosure provides a kit comprising two or more separate pharmaceutical compositions, at least one of which contains a compound of formula (I). The other pharmaceutical composition may contain one of the suitable co-agents. In one embodiment, the kit comprises means for separately retaining said compositions, such as a container, divided bottle, or divided foil packet. An example of such a kit is a blister pack, as typically used for the packaging of tablets, capsules and the like.

The kit disclosed herein may be used for administering different dosage forms, for example, oral and parenteral, for administering the separate compositions at different dosage intervals, or for titrating the separate compositions against one another. To assist compliance, the provided kit typically comprises directions for administration.

In the combination therapies disclosed herein, the provided compound and the therapeutic co-agent may be manufactured and/or formulated by the same or different manufacturers. Moreover, the provided compound and the therapeutic co-agent may be brought together into a combination therapy: (i) prior to release of the combination product to physicians (e.g. in the case of a kit comprising the provided compound and the other therapeutic agent); (ii) by the physician themselves (or under the guidance of the physician) shortly before administration; (iii) in the patient themselves, e.g. during sequential administration of the provided compound and the therapeutic co-agent.

Accordingly, the disclosure provides the use of a compound of formula (I) for treating a viral infection caused by an orthomyxovirus, particularly influenza, which may be Influenza A, Influenza B or Influenza C, wherein the medicament is prepared for administration with a therapeutic co-agent. Typically in the methods of using the provided compounds, the serotype of influenza is not identified before treatment. The disclosure also provides the use of therapeutic co-agent for treating a disease or condition, wherein the medicament is administered with a compound of formula (I).

The disclosure also provides a compound of formula (I) for use in a method of treating a viral infection caused by an orthomyxovirus, particularly Influenza A, Influenza B or Influenza C, wherein the compound of formula (I) is prepared for administration with a therapeutic co-agent. The disclosure also provides another therapeutic co-agent for use in a method of treating a viral infection caused by an orthomyxovirus, particularly influenza, e.g., Influenza A, Influenza B or Influenza C, wherein the therapeutic co-agent is prepared for administration with a compound of formula (I). The disclosure also provides a compound of formula (I) for use in a method of treating a viral infection caused by an orthomyxovirus, particularly Influenza A, Influenza B or Influenza C, wherein the compound of formula (I) is administered with a therapeutic co-agent. The disclosure also provides a therapeutic co-agent for use in a method of treating a viral infection caused by an orthomyxovirus, particularly Influenza A, Influenza B or Influenza C, wherein the a therapeutic co-agent is administered with a compound of formula (I).

The disclosure also provides the use of a compound of formula (I) for treating a viral infection caused by an orthomyxovirus, particularly influenza, e.g., Influenza A, Influenza B or Influenza C, wherein the patient has previously (e.g. within 24 hours) been treated with another therapeutic agent. The disclosure also provides the use of another therapeutic agent for treating a viral infection caused by an orthomyxovirus, particularly Influenza A, Influenza B or Influenza C, wherein the patient has previously (e.g. within 24 hours) been treated with a compound of formula (I).

In one embodiment, the therapeutic co-agent is selected from antivirals purported to be useful for treating infections caused by influenza viruses, such as neuraminidase inhibitors including oseltamivir, peramivir, zanamivir and/or laninamivir, and adamantanes such as amantadine and/or rimantadine.

### EXAMPLES

The following examples are intended for illustrative purposes only and are not to be construed as limiting the scope of the embodiments. Temperatures are given in degrees Celsius. If not mentioned otherwise, all evaporations are performed under reduced pressure, typically between about 15 mm Hg and 100 mm Hg (about 20-133 mbar). The structure of final products, intermediates and starting materials is confirmed by standard analytical methods, e.g., microanalysis and spectroscopic characteristics, e.g., MS, IR and NMR. Abbreviations used are those conventional in the art.

All starting materials, building blocks, reagents, acids, bases, dehydrating agents, solvents, and catalysts utilized to synthesize the provided compounds are either commercially available or may be produced by organic synthesis methods known to one of ordinary skill in the art (Houben-Weyl 4th Ed. 1952, Methods of Organic Synthesis, Thieme, Volume 21). Further, the provided compounds may be produced by organic synthesis methods known to one of ordinary skill in the art in view of the following examples.

### Abbreviations

ATP adenosine 5′-triphosphate
Bn benzyl
BOC tertiary butyl carboxy
br broad
BSA bovine serum albumin
d doublet
dd doublet of doublets
DCM dichloromethane
DEAD diethyl azodicarboxylate
DBAD di-tert-butyl azodicarboxylate
DIBAL-H diisobutylaluminum hydride
DIEA diethylisopropylamine
DME 1,4-dimethoxyethane
DMF N,N-dimethylformamide
DMSO dimethylsulfoxide
DTT dithiothreitol
EDTA ethylenediamine tetraacetic acid
ESI electrospray ionization
EtOAc or EA ethyl acetate
FCC flash column chromatography
h hour(s)
HBTU 1-[bis(dimethylamino)methylene]-1H-benzotriazoliumhexafluorophosphate(1-) 3-oxide
HOBt 1-hydroxy-7-azabenzotriazole
HPLC high pressure liquid chromatography
IR infrared spectroscopy
LCMS liquid chromatography and mass spectrometry
MeOH methanol
MS mass spectrometry
MW microwave
m multiplet
min minutes
mL milliliter(s)
m/z mass to charge ratio
NBS N-bromosuccinimide
NCS N-chlorosuccinimide
NMP N-methyl pyrrolidinone
NMR nuclear magnetic resonance
ppm parts per million
PyBOP benzotriazol-1-yloxytripyrrolidinophosphonium hexafluorophosphate
rac racemic
rt room temperature
s singlet
SEM (2-(trimethylsilyl)ethoxy)methyl
t triplet
TBDMS t-butyldimethylsilyl
TBDPS t-butyldiphenylsilyl
TFA trifluoroacetic acid
THF tetrahydrofuran
Tris·HCl aminotris(hydroxymethyl)methane hydrochloride

### Synthesis of Exemplary Compounds

### Example 1: 5-(4-(morpholinomethyl)phenyl)-2-oxo-6-(trifluoromethyl)-1,2-dihydropyridine-3-carboxamide (1)

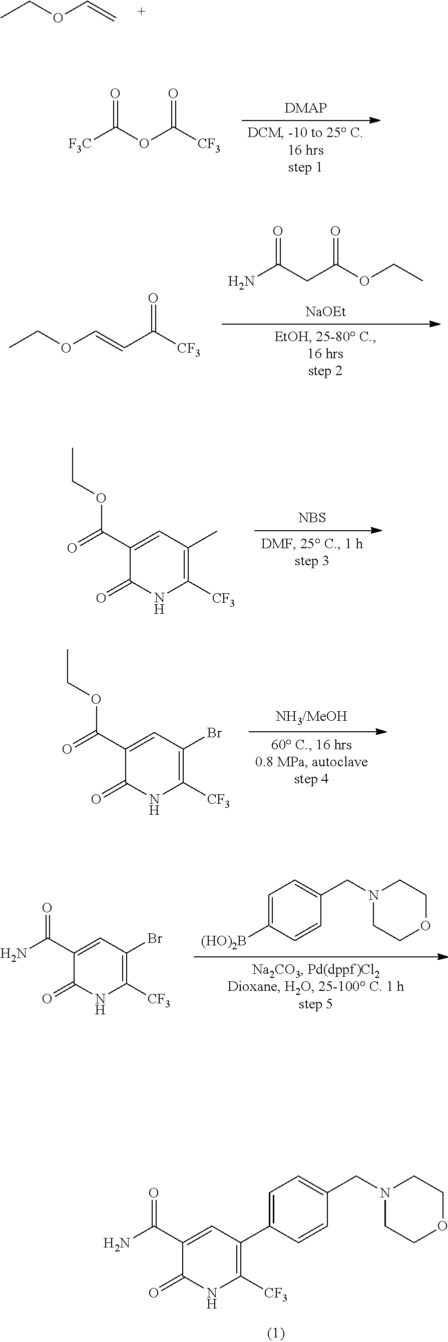

Step 1: To a solution of ethoxyethene (100 g, 1.389 mol) in DCM (1.0 L) was added 2,2,2-trifluoroacetic anhydride (306 g, 1.457 mol) and DMAP (10 g, 81.9 mmol) in turns at −10° C. Then the mixture was stirred at 25° C. for 16 hrs. The mixture was concentrated under vacuum. The residue was purified by column (PE:EA=1:0˜30:1) to give (E)-4-ethoxy-1,1,1-trifluorobut-3-en-2-one. ¹H NMR: (CDCl3, 400 MHz), δ=7.92 (d, J=12.30 Hz, 1H) 5.87 (d, J=12.30 Hz, 1H) 4.12-4.17 (q, 2H) 1.43 (t, J=7.09 Hz, 3H).

Step 2: To a solution of (E)-4-ethoxy-1,1,1-trifluorobut-3-en-2-one (step 1) (50 g, 298 mmol) and NaOEt (40 g, 596 mmol) in EtOH (500 mL) was added ethyl 3-amino-3-oxopropanoate (43 g, 358 mmol) at 25° C. Then the mixture was stirred at 80° C. for 16 hrs. The reaction mixture was poured into water (1.0 L) and then extracted with EA (500 mL×2). The combined organic layers were dried over anhydrous Na₂SO₄, filtered and concentrated to give crude product. The residue was purified by column (PE:EA=10:1˜1:1) to give ethyl 2-oxo-6-(trifluoromethyl)-1,2-dihydropyridine-3-carboxylate. ¹H NMR (400 MHz, CHLOROFORM-d) δ=8.31 (d, J=6.8 Hz, 1H), 7.23 (d, J=6.8 Hz, 1H), 4.42 (m, 2H), 1.36 (t, J=6.4 Hz, 3H); LCMS: Rt 0.702 min, MS m/z 236.0 (M+H).

Step 3: To a solution of ethyl 2-oxo-6-(trifluoromethyl)-1,2-dihydropyridine-3-carboxylate (10 g, 42.6 mmol) in DMF (50 ml) was added NBS (9.1 g, 51.1 mmol) at 25° C. The mixture was stirred at 25° C. for 1 h. The mixture was washed with H₂O (100 mL) and extracted with EA (100 mL×2). The combined organic layers were dried over anhydrous Na₂SO₄, filtered and concentrated to dryness. The residue was purified by column (PE/EA=10/1) to give ethyl 5-bromo-2-oxo-6-(trifluoromethyl)-1,2-dihydropyridine-3-carboxylate. ¹H NMR (400 MHz, DMSO-d6) δ=8.43 (s, 1H), 4.33-4.28 (m, 2H), 1.30 (t, J=8 Hz, 3H) LCMS: Rt 0.864 min, MS m/z 314.0 (M+H).

Step 4: A solution of ethyl 5-bromo-2-oxo-6-(trifluoromethyl)-1,2-dihydropyridine-3-carboxylate (5 g, 16.0 mmol) in NH₃/MeOH (50 ml) was stirred in an autoclave at 60° C. for 16 hrs under 0.8 MPa. The reaction mixture was concentrated to give 5-bromo-2-oxo-6-(trifluoromethyl)-1,2-dihydropyridine-3-carboxamide, which was used for next step without further purification. ¹H NMR: (DMSO, 400 MHz), δ=10.41 (s, 1H), 8.09 (s, 1H), 7.22 (s, 2H); LCMS: Rt 0.665 min, MS m/z 286.9 (M+H).

Step 5: 5-bromo-2-oxo-6-(trifluoromethyl)-1,2-dihydropyridine-3-carboxamide (100 mg, 0.35 mmol, (4-(morpholinomethyl)phenyl)boronic acid (117 mg, 0.529 mmol) and PdCl₂(dppf)-CH₂Cl₂ adduct (28.7 mg, 0.035 mmol) were added to a microwave vial with a stir bar and then purged with nitrogen. 1,4-dioxane (1170 μl) was then added, followed by sodium carbonate (2.0 M in water) (877 μl, 1.754 mmol). The vial was sealed and heated in the microwave at 100° C. for 60 min. The mixture was poured into water (5 mL) and extracted with EA (5 mL×2). The combined organic layers were dried over anhydrous Na₂SO₄, filtered, and concentrated to dryness. The residue was purified by Prep-HPLC under acidic conditions (MeCN/water with a TFA) to give 5-(4-(morpholinomethyl)phenyl)-2-oxo-6-(trifluoromethyl)-1,2-dihydropyridine-3-carboxamide (1). ¹H NMR: (400 MHz, DMSO-d6) δ 8.58 (s, 1H), 8.28 (s, 1H), 8.11 (s, 1H), 7.44-7.36 (m, 2H), 7.35-7.27 (m, 2H), 3.60 (t, J=4.7 Hz, 4H), 2.40 (t, J=4.7 Hz, 4H). LCMS: Rt=0.43 min, m/z=382.3 (M+H).

### Example 2: 5-(4-(hydroxymethyl)phenyl)-2-oxo-6-(trifluoromethyl)-1,2-dihydropyridine-3-carboxamide (2)

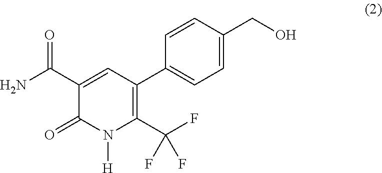 [SMILES: NC(=O)c1cc(-c2ccc(CO)cc2)c(C(F)(F)F)[nH]c1=O]

5-(4-(hydroxymethyl)phenyl)-2-oxo-6-(trifluoromethyl)-1,2-dihydropyridine-3-carboxamide (2) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(hydroxymethyl)phenyl)boronic acid. ¹H NMR: (400 MHz, DMSO-d6) δ 9.55 (bs, 1H), 8.06 (s, 1H), 7.62 (t, J=9.8 Hz, 1H), 7.43-7.30 (m, 2H), 7.24 (d, J=7.9 Hz, 2H), 7.17-6.92 (m, 1H), 5.22 (t, J=6.0 Hz, 1H), 4.54 (d, J=4.8 Hz, 2H). LCMS: Rt=0.50 min, m/z=313.1 (M+H).

### Example 3: 5-(4-hydroxyphenyl)-2-oxo-6-(trifluoromethyl)-1,2-dihydropyridine-3-carboxamide (3)

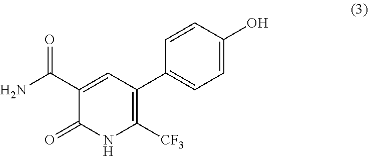 [SMILES: NC(=O)c1cc(-c2ccc(O)cc2)c(C(F)(F)F)[nH]c1=O]

5-(4-hydroxyphenyl)-2-oxo-6-(trifluoromethyl)-1,2-dihydropyridine-3-carboxamide (3) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-hydroxyphenyl)boronic acid. ¹H NMR: (400 MHz, DMSO-d6) δ 9.83 (s, 1H), 9.03-8.69 (m, 1H), 8.24 (s, 1H), 8.10-7.97 (m, 1H), 7.12 (d, J=8.4 Hz, 2H), 6.86 (d, J=8.4 Hz, 1H). LCMS: Rt=0.68 min, m/z=299.0 (M+H).

### Example 4: 5-(4-(benzyloxy)phenyl)-2-oxo-6-(trifluoromethyl)-1,2-dihydropyridine-3-carboxamide (4)

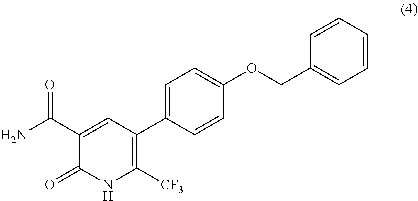 [SMILES: NC(=O)c1cc(-c2ccc(OCc3ccccc3)cc2)c(C(F)(F)F)[nH]c1=O]

5-(4-(benzyloxy)phenyl)-2-oxo-6-(trifluoromethyl)-1,2-dihydropyridine-3-carboxamide (4) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(benzyloxy)phenyl)boronic acid. ¹H NMR: (400 MHz, DMSO-d6) δ 8.59 (s, 1H), 8.43-8.41 (d, J=8, 1H), 8.25 (s, 1H), 8.11 (s, 2H), 8.50-8.47 (m, 3H), 7.43-7.41 (m, 2H), 7.39-7.34 (m, 1H), 7.28-7.26 (m, 2H), 7.10-7.08 (m, 2H), 5.10 (s, 2H). LCMS: Rt=0.86 min, m/z=389.2 (M+H).

### Example 5: 2-oxo-5-(4-(pyrrolidin-1-ylmethyl)phenyl)-6-(trifluoromethyl)-1,2-dihydropyridine-3-carboxamide (5)

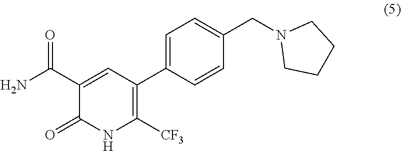 [SMILES: NC(=O)c1cc(-c2ccc(CN3CCCC3)cc2)c(C(F)(F)F)[nH]c1=O]

2-oxo-5-(4-(pyrrolidin-1-ylmethyl)phenyl)-6-(trifluoromethyl)-1,2-dihydropyridine-3-carboxamide (5) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(pyrrolidin-1-ylmethyl)phenyl)boronic acid. ¹H NMR: (400 MHz, DMSO-d6) δ 9.86 (br, s, 1H), 7.99 (s, 1H), 7.45 (d, J=8.0 2H), 7.28 (d, J=8.0 2H), 4.03 (s, 2H), 2.89 (m, 4H), 1.84 (m, 4H). LCMS: Rt=0.53 min, m/z=366.2 (M+H).

### Example 6: 2-oxo-5-(4-phenoxyphenyl)-6-(trifluoromethyl)-1,2-dihydropyridine-3-carboxamide (6)

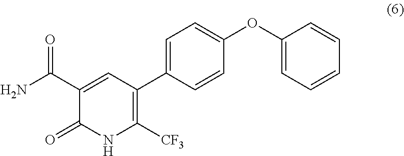 [SMILES: NC(=O)c1cc(-c2ccc(Oc3ccccc3)cc2)c(C(F)(F)F)[nH]c1=O]

2-oxo-5-(4-phenoxyphenyl)-6-(trifluoromethyl)-1,2-dihydropyridine-3-carboxamide (6) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-phenoxyphenyl)boronic acid. ¹H NMR: (400 MHz, DMSO-d6) δ 8.46 (s, 1H), 8.30 (s, 1H), 8.15 (s, 1H), 7.42 (d, J=8.0 Hz, 2H), 7.36 (d, J=8.0 Hz, 2H), 7.19 (t, J=7.2 Hz, 1H), 7.07 (d, J=8.4 Hz, 4H). LCMS: Rt=0.85 min, m/z=375.2 (M+H).

### Example 7: 5-(3-aminophenyl)-2-oxo-6-(trifluoromethyl)-1,2-dihydropyridine-3-carboxamide (7)

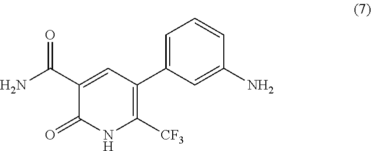 [SMILES: NC(=O)c1cc(-c2cccc(N)c2)c(C(F)(F)F)[nH]c1=O]

5-(3-aminophenyl)-2-oxo-6-(trifluoromethyl)-1,2-dihydropyridine-3-carboxamide (7) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (3-aminophenyl)boronic acid. ¹H NMR: (400 MHz, DMSO-d6) δ 8.17 (s, 1H), 7.05 (t, J=7.8 Hz, 1H), 6.58 (d, J=7.9 Hz, 1H), 6.51-6.46 (m, 1H), 6.45-6.38 (m, 1H). LCMS: Rt=0.61 min, m/z=298.1 (M+H).

### Example 8: 5-(3-cyanophenyl)-2-oxo-6-(trifluoromethyl)-1,2-dihydropyridine-3-carboxamide (8)

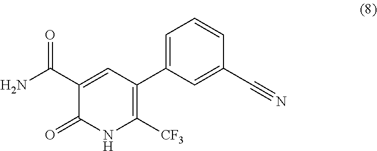 [SMILES: N#Cc1cccc(-c2cc(C(N)=O)c(=O)[nH]c2C(F)(F)F)c1]

5-(3-cyanophenyl)-2-oxo-6-(trifluoromethyl)-1,2-dihydropyridine-3-carboxamide (8) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (3-cyanophenyl)boronic acid. ¹H NMR: (400 MHz, DMSO-d6) δ 13.93-13.72 (m, 1H), 8.51-8.40 (m, 1H), 8.33 (s, 1H), 8.27-8.21 (m, 1H), 7.99-7.88 (m, 2H), 7.72 (s, 2H). LCMS: Rt=0.68 min, m/z=308.2 (M+H).

### Example 9: 5-(3-hydroxyphenyl)-2-oxo-6-(trifluoromethyl)-1,2-dihydropyridine-3-carboxamide (9)

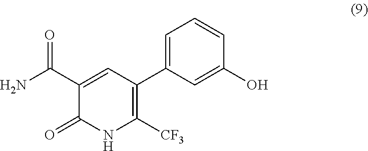 [SMILES: NC(=O)c1cc(-c2cccc(O)c2)c(C(F)(F)F)[nH]c1=O]

5-(3-hydroxyphenyl)-2-oxo-6-(trifluoromethyl)-1,2-dihydropyridine-3-carboxamide (9) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (3-hydroxyphenyl)boronic acid. ¹H NMR: (400 MHz, DMSO-d6) δ 13.81-13.62 (m, 1H), 9.66 (s, 1H), 8.48 (s, 1H), 8.29 (s, 1H), 8.20 (s, 1H), 7.26 (t, J=7.8 Hz, 1H), 6.85 (s, 1H), 6.79-6.66 (m, 2H). LCMS: Rt=0.59 min, m/z=299.2 (M+H).

### Example 10: 5-(3-formamidophenyl)-2-oxo-6-(trifluoromethyl)-1,2-dihydropyridine-3-carboxamide (10)

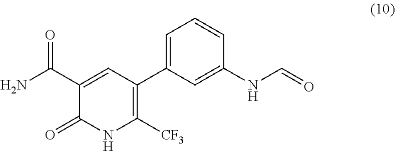 [SMILES: NC(=O)c1cc(-c2cccc(NC=O)c2)c(C(F)(F)F)[nH]c1=O]

5-(3-formamidophenyl)-2-oxo-6-(trifluoromethyl)-1,2-dihydropyridine-3-carboxamide (10) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (3-formamidophenyl)boronic acid. ¹H NMR: (400 MHz, DMSO-d6) δ 10.44-10.33 (m, 1H), 8.57-8.38 (m, 2H), 8.31 (m, 2H), 8.21 (s., 1H), 7.64 (s, 2H), 7.49-7.36 (m, 1H), 7.10-7.04 (m, 1H). LCMS: Rt=0.58 min, m/z=326.0 (M+H).

### Example 11: 5-(4-(methylsulfonamido)phenyl)-2-oxo-6-(trifluoromethyl)-1,2-dihydropyridine-3-carboxamide (11)

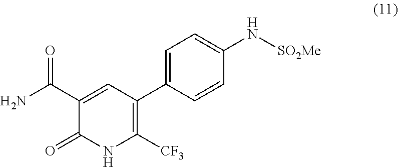 [SMILES: CS(=O)(=O)Nc1ccc(-c2cc(C(N)=O)c(=O)[nH]c2C(F)(F)F)cc1]

5-(4-(methylsulfonamido)phenyl)-2-oxo-6-(trifluoromethyl)-1,2-dihydropyridine-3-carboxamide (11) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(methylsulfonamido)phenyl)boronic acid. LCMS: Rt=0.61 min, m/z=376.1 (M+H).

### Example 12: 5-(4-(difluoromethoxy)phenyl)-2-oxo-6-(trifluoromethyl)-1,2-dihydropyridine-3-carboxamide (12)

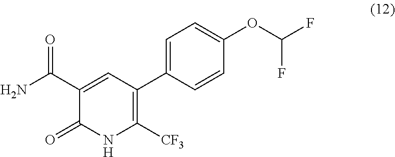 [SMILES: NC(=O)c1cc(-c2ccc(OC(F)F)cc2)c(C(F)(F)F)[nH]c1=O]

5-(4-(difluoromethoxy)phenyl)-2-oxo-6-(trifluoromethyl)-1,2-dihydropyridine-3-carboxamide (12) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(difluoromethoxy)phenyl)boronic acid. LCMS: Rt=0.77 min, m/z=349.0 (M+H).

### Example 13: 5-(4-(methoxymethoxy)phenyl)-2-oxo-6-(trifluoromethyl)-1,2-dihydropyridine-3-carboxamide (13)

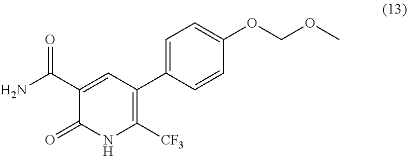 [SMILES: COCOc1ccc(-c2cc(C(N)=O)c(=O)[nH]c2C(F)(F)F)cc1]

5-(4-(methoxymethoxy)phenyl)-2-oxo-6-(trifluoromethyl)-1,2-dihydropyridine-3-carboxamide (13) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(methoxymethoxy)phenyl)boronic acid. LCMS: Rt=2.94 min, m/z=343.0 (M+H).

### Example 14: 5-(3-fluoro-4-methoxyphenyl)-2-oxo-6-(trifluoromethyl)-1,2-dihydropyridine-3-carboxamide (14)

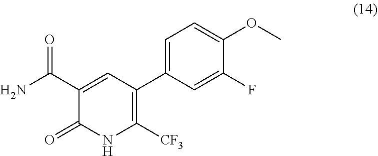 [SMILES: COc1ccc(-c2cc(C(N)=O)c(=O)[nH]c2C(F)(F)F)cc1F]

5-(3-fluoro-4-methoxyphenyl)-2-oxo-6-(trifluoromethyl)-1,2-dihydropyridine-3-carboxamide (14) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (3-fluoro-4-methoxyphenyl)boronic acid. LCMS: Rt=2.02 min, m/z=329.0 (M−H).

### Example 15: 5-(4-(methoxymethyl)phenyl)-2-oxo-6-(trifluoromethyl)-1,2-dihydropyridine-3-carboxamide (15)

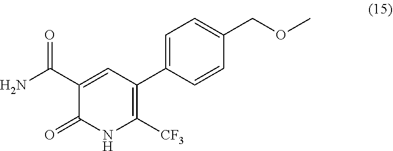 [SMILES: COCc1ccc(-c2cc(C(N)=O)c(=O)[nH]c2C(F)(F)F)cc1]

5-(4-(methoxymethyl)phenyl)-2-oxo-6-(trifluoromethyl)-1,2-dihydropyridine-3-carboxamide (15) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(methoxymethyl)phenyl)boronic acid. LCMS: Rt=2.91 min, m/z=327.0 (M+H).

### Example 16: 2-oxo-5-phenyl-6-(trifluoromethyl)-1,2-dihydropyridine-3-carboxamide (16)

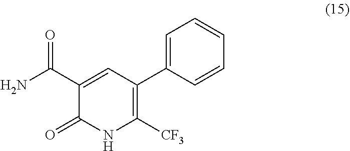 [SMILES: NC(=O)c1cc(-c2ccccc2)c(C(F)(F)F)[nH]c1=O]

2-oxo-5-phenyl-6-(trifluoromethyl)-1,2-dihydropyridine-3-carboxamide (16) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with phenylboronic acid. LCMS: Rt=0.96 min, m/z=283.5 (M+H).

### Example 17: 5-(4-isopropoxyphenyl)-2-oxo-6-(trifluoromethyl)-1,2-dihydropyridine-3-carboxamide (17)

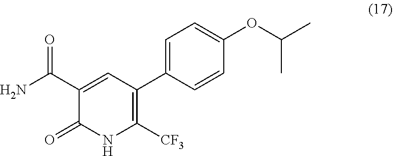 [SMILES: CC(C)Oc1ccc(-c2cc(C(N)=O)c(=O)[nH]c2C(F)(F)F)cc1]

5-(4-isopropoxyphenyl)-2-oxo-6-(trifluoromethyl)-1,2-dihydropyridine-3-carboxamide (17) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-isopropoxyphenyl)boronic acid. LCMS: Rt=3.20 min, m/z=339.0 (M−H).

### Example 18: 5-(3-chloro-4-methoxyphenyl)-2-oxo-6-(trifluoromethyl)-1,2-dihydropyridine-3-carboxamide (18)

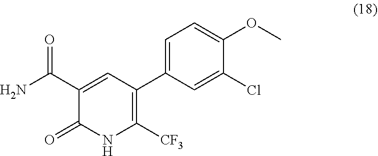 [SMILES: COc1ccc(-c2cc(C(N)=O)c(=O)[nH]c2C(F)(F)F)cc1Cl]

5-(3-chloro-4-methoxyphenyl)-2-oxo-6-(trifluoromethyl)-1,2-dihydropyridine-3-carboxamide (18) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (3-chloro-4-methoxyphenyl)boronic acid. LCMS: Rt=2.08 min, m/z=345.0 (M−H).

### Example 19: 5-(4-(methylcarbamoyl)phenyl)-2-oxo-6-(trifluoromethyl)-1,2-dihydropyridine-3-carboxamide (19)

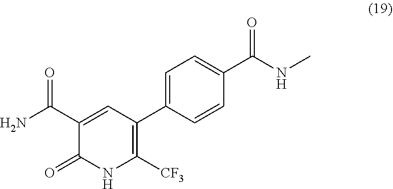 [SMILES: CNC(=O)c1ccc(-c2cc(C(N)=O)c(=O)[nH]c2C(F)(F)F)cc1]

5-(4-(methylcarbamoyl)phenyl)-2-oxo-6-(trifluoromethyl)-1,2-dihydropyridine-3-carboxamide (19) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(methylcarbamoyl)phenyl)boronic acid. LCMS: Rt=2.40 min, m/z=340.0 (M+H).

### Example 20: 5-(4-(cyanomethyl)phenyl)-2-oxo-6-(trifluoromethyl)-1,2-dihydropyridine-3-carboxamide (20)

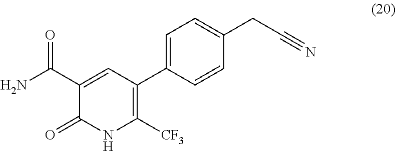 [SMILES: N#CCc1ccc(-c2cc(C(N)=O)c(=O)[nH]c2C(F)(F)F)cc1]

5-(4-(cyanomethyl)phenyl)-2-oxo-6-(trifluoromethyl)-1,2-dihydropyridine-3-carboxamide (20) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(cyanomethyl)phenyl)boronic acid. LCMS: Rt=2.80 min, m/z=322.0 (M+H).

### Example 21: 5-(4-(3-(dimethylamino)propoxy)phenyl)-2-oxo-6-(trifluoromethyl)-1,2-dihydropyridine-3-carboxamide (21)

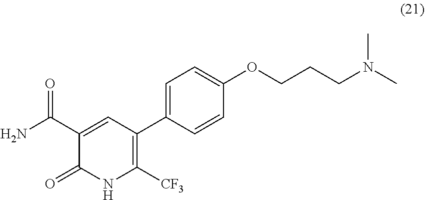 [SMILES: CN(C)CCCOc1ccc(-c2cc(C(N)=O)c(=O)[nH]c2C(F)(F)F)cc1]

5-(4-(3-(dimethylamino)propoxy)phenyl)-2-oxo-6-(trifluoromethyl)-1,2-dihydropyridine-3-carboxamide (21) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (4-(3-(dimethylamino)propoxy)phenyl)boronic acid. LCMS: Rt=2.13 min, m/z=384.0 (M+H).

### Example 22: 5-(3-(cyanomethyl)phenyl)-2-oxo-6-(trifluoromethyl)-1,2-dihydropyridine-3-carboxamide (22)

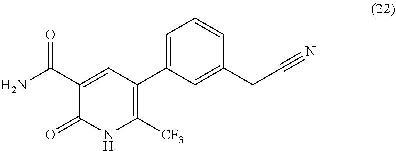 [SMILES: N#CCc1cccc(-c2cc(C(N)=O)c(=O)[nH]c2C(F)(F)F)c1]

5-(3-(cyanomethyl)phenyl)-2-oxo-6-(trifluoromethyl)-1,2-dihydropyridine-3-carboxamide (22) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (3-(cyanomethyl)phenyl)boronic acid. LCMS: Rt=2.77 min, m/z=320.0 (M−H).

### Example 23: 5-(3-(morpholinomethyl)phenyl)-2-oxo-6-(trifluoromethyl)-1,2-dihydropyridine-3-carboxamide (23)

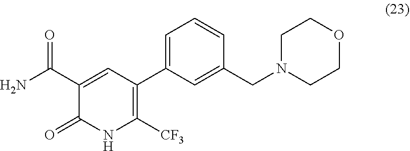 [SMILES: NC(=O)c1cc(-c2cccc(CN3CCOCC3)c2)c(C(F)(F)F)[nH]c1=O]

5-(3-(morpholinomethyl)phenyl)-2-oxo-6-(trifluoromethyl)-1,2-dihydropyridine-3-carboxamide (23) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (3-(morpholinomethyl)phenyl)boronic acid. LCMS: Rt=1.95 min, m/z=380.0 (M−H).

### Example 24: 2-oxo-5-(m-tolyl)-6-(trifluoromethyl)-1,2-dihydropyridine-3-carboxamide (24)

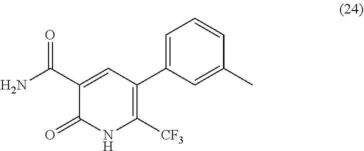 [SMILES: Cc1cccc(-c2cc(C(N)=O)c(=O)[nH]c2C(F)(F)F)c1]

2-oxo-5-(m-tolyl)-6-(trifluoromethyl)-1,2-dihydropyridine-3-carboxamide (24) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with m-tolylboronic acid. LCMS: Rt=3.04 min, m/z=295.2 (M−H).

### Example 25: 5-(3-(hydroxymethyl)phenyl)-2-oxo-6-(trifluoromethyl)-1,2-dihydropyridine-3-carboxamide (25)

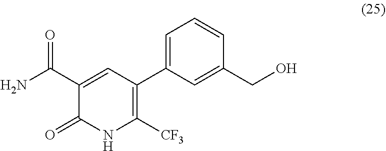 [SMILES: NC(=O)c1cc(-c2cccc(CO)c2)c(C(F)(F)F)[nH]c1=O]

5-(3-(hydroxymethyl)phenyl)-2-oxo-6-(trifluoromethyl)-1,2-dihydropyridine-3-carboxamide (25) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with (3-(hydroxymethyl)phenyl)boronic acid. LCMS: Rt=1.17 min, m/z=311.0 (M−H).

### Example 26: 2-oxo-5-(p-tolyl)-6-(trifluoromethyl)-1,2-dihydropyridine-3-carboxamide (26)

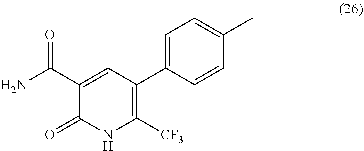 [SMILES: Cc1ccc(-c2cc(C(N)=O)c(=O)[nH]c2C(F)(F)F)cc1]

2-oxo-5-(p-tolyl)-6-(trifluoromethyl)-1,2-dihydropyridine-3-carboxamide (26) was made using a procedure similar to that used to make 5-(4-(morpholinomethyl)phenyl)-2-oxo-6-(trifluoromethyl)-1,2-dihydropyridine-3-carboxamide (1), except (4-(morpholinomethyl)phenyl)boronic acid was replaced with p-tolylboronic acid. LCMS: Rt=3.02 min, m/z=295.0 (M−H).

### Example 27: 5-(4-((3-methylmorpholino)methyl)phenyl)-2-oxo-6-(trifluoromethyl)-1,2-dihydropyridine-3-carboxamide (27)

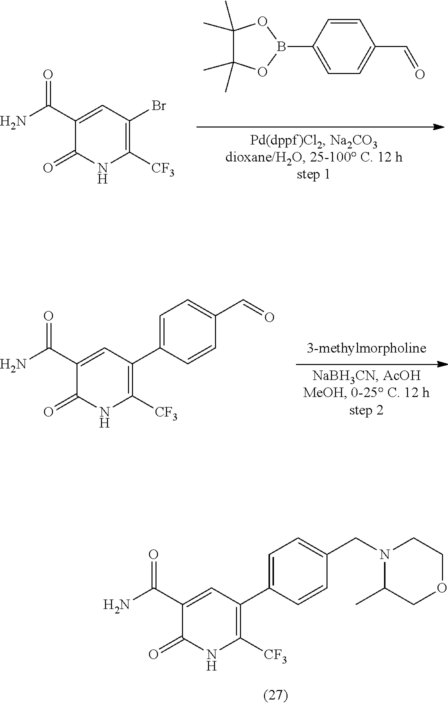 [SMILES: NC(=O)c1cc(Br)c(C(F)(F)F)[nH]c1=O ; CC1(C)OB(c2ccc(C=O)cc2)OC1(C)C ; "Pd(dppf)Cl2, Na2CO3dioxane/H2O, 25-100° C. 12 hstep 1" ; NC(=O)c1cc(-c2ccc(C=O)cc2)c(C(F)(F)F)[nH]c1=O ; "NaBH3CN, AcOHMeOH, 0-25° C. 12 hstep 2" ; CC1COCCN1Cc1ccc(-c2cc(C(N)=O)c(=O)[nH]c2C(F)(F)F)cc1]

Step 1: To a solution of 5-bromo-2-oxo-6-(trifluoromethyl)-1,2-dihydropyridine-3-carboxamide (see Example 1) (3.0 g, 10.5 mmol) in dioxane (10 mL) and H₂O (2 mL) was added 4-(4,4,5,5-tetramethyl-1,3,2-dioxaborolan-2-yl)benzaldehyde (2.9 g, 12.6 mmol), Na₂CO₃ (2.2 g, 21.0 mmol) and Pd(dppf)Cl₂—CH₂Cl₂ adduct (0.4 g, 0.5 mmol) at 25° C. under N₂. The mixture was stirred at 100° C. for 12 hrs. The mixture was washed with H₂O (10 mL) and extracted with EA (10 mL×2). The combined organic layers were dried over anhydrous Na₂SO₄, filtered and concentrated to dryness. The residue was purified by Prep-HPLC under the acidic conditions (MeCN/water with TFA) to give 5-(4-formylphenyl)-2-oxo-6-(trifluoromethyl)-1,2-dihydropyridine-3-carboxamide. ¹H NMR: (400 MHz, DMSO) δ=10.07 (s, 1H), 8.43 (br, s, 1H), 8.32 (s, 1H), 8.20 (br, s, 1H), 8.01 (d, J=8.0 Hz, 2H), 7.61 (d, J=8.0 Hz, 2H). LCMS: Rt=0.66 min, m/z=311.0 (M+H).

Step 2: To a solution of 5-(4-formylphenyl)-2-oxo-6-(trifluoromethyl)-1,2-dihydropyridine-3-carboxamide (200 mg, 0.64 mmol) in MeOH (5 mL) was added 3-methylmorpholine (130 mg, 1.29 mmol) and CH₃COOH (40 mg, 0.32 mmol) at 25° C. The mixture was stirred at 25° C. for 1 hr. Then NaBH₃CN (120 mg, 1.92 mmol) was added at 0° C. The mixture was stirred at 25° C. for 11 hrs. The mixture was washed with H₂O (10 mL) and extracted with EA (10 mL×2). The combined organic layers were dried over anhydrous Na₂SO₄, filtered and concentrated. The residue was purified by Prep-HPLC under basic conditions (MeCN/water with NH₃·H2O) added “basic” method) to give 5-(4-((3-methylmorpholino)methyl)phenyl)-2-oxo-6-(trifluoromethyl)-1,2-dihydropyridine-3-carboxamide (27). ¹H NMR (400 MHz, DMSO) δ=7.88 (s, 1H), 7.30 (d, J=8.0, 2H), 7.18 (d, J=8.0, 2H), 4.02 (d, J=13.2, 1H), 3.64 (m, 2H), 3.61-3.60 (m, 2H), 3.33 (s, 1H), 3.16-3.12 (m, 2H), 2.49-2.48 (m, 2H). 2.10-2.07 (m, 1H), 1.02-1.00 (d, J=6.4, 3H). LCMS: Rt=0.92 min, m/z=396.1 (M+H).

### Example 28: 2-oxo-5-(4-(thiomorpholinomethyl)phenyl)-6-(trifluoromethyl)-1,2-dihydropyridine-3-carboxamide (28)

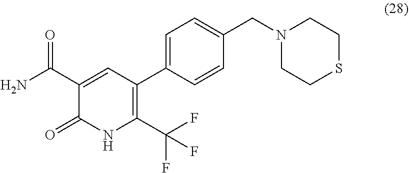 [SMILES: NC(=O)c1cc(-c2ccc(CN3CCSCC3)cc2)c(C(F)(F)F)[nH]c1=O]

2-oxo-5-(4-(thiomorpholinomethyl)phenyl)-6-(trifluoromethyl)-1,2-dihydropyridine-3-carboxamide (28) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with thiomorpholine. ¹H NMR (400 MHz, DMSO) δ=8.01 (s, 1H), 7.46 (br s, 1H), 7.32 (d, J=8.0 Hz, 2H), 7.22 (d, J=8.0 Hz, 2H), 3.53 (s, 2H), 2.63-2.61 (m, 8H), LCMS: Rt=0.99 min, m/z=398.1 (M+H).

### Example 29: 5-(4-((2-methylmorpholino)methyl)phenyl)-2-oxo-6-(trifluoromethyl)-1,2-dihydropyridine-3-carboxamide (29)

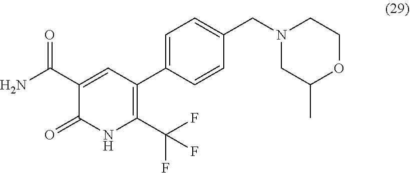 [SMILES: CC1CN(Cc2ccc(-c3cc(C(N)=O)c(=O)[nH]c3C(F)(F)F)cc2)CCO1]

5-(4-((2-methylmorpholino)methyl)phenyl)-2-oxo-6-(trifluoromethyl)-1,2-dihydropyridine-3-carboxamide (29) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with 2-methylmorpholine. ¹H NMR (400 MHz, DMSO) δ=7.86 (s, 1H), 7.29 (d, J=8.0, 2H), 7.18 (d, J=8.0, 2H), 3.73 (m, 1H), 3.51 (m, 2H), 3.46 (s, 2H), 2.70 (m, 1H), 2.67 (m, 1H), 2.49-2.48 (m, 1H), 2.02-2.01 (m, 1H), 1.71 (t, J=10.8, 1H), 1.02-1.01 (d, J=6.0, 3H). LCMS: Rt=0.78 min, m/z=396.1 (M+H).

### Example 30: 5-(4-((7-oxa-4-azaspiro[2.5]octan-4-yl)methyl)phenyl)-2-oxo-6-(trifluoromethyl)-1,2-dihydropyridine-3-carboxamide (30)

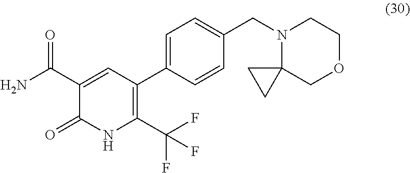 [SMILES: NC(=O)c1cc(-c2ccc(CN3CCOCC34CC4)cc2)c(C(F)(F)F)[nH]c1=O]

5-(4-((7-oxa-4-azaspiro[2.5]octan-4-yl)methyl)phenyl)-2-oxo-6-(trifluoromethyl)-1,2-dihydropyridine-3-carboxamide (30) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with 7-oxa-4-azaspiro[2.5]octane. ¹H NMR (400 MHz, DMSO) δ=9.15 (br. s, 1H), 8.19 (s, 1H), 8.14 (s, 1H), 7.84 (s, 1H), 7.33 (d, J=8.0 Hz, 2H), 7.23 (d, J=8.0 Hz, 2H), 3.84 (s, 2H), 3.67-3.65 (m, 2H), 3.55 (br. s., 2H), 2.69-2.67 (m, 2H), 0.69-0.67 (m, 2H), 0.50-0.48 (m, 2H). LCMS: Rt=0.65 min, m/z=408.1 (M+H).

### Example 31: 5-(4-(((1R,5S)-8-oxa-3-azabicyclo[3.2.1]octan-3-yl)methyl)phenyl)-2-oxo-6-(trifluoromethyl)-1,2-dihydropyridine-3-carboxamide (31)

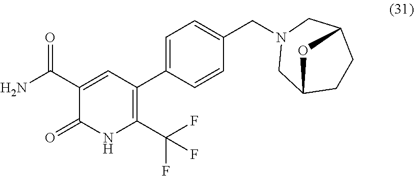 [SMILES: NC(=O)c1cc(-c2ccc(CN3C[C@H]4CC[C@@H](C3)O4)cc2)c(C(F)(F)F)[nH]c1=O]

5-(4-(((1R,5S)-8-oxa-3-azabicyclo[3.2.1]octan-3-yl)methyl)phenyl)-2-oxo-6-(trifluoromethyl)-1,2-dihydropyridine-3-carboxamide (31) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with (1R,5S)-8-oxa-3-azabicyclo[3.2.1]octane. ¹H NMR (400 MHz, DMSO) δ=7.82 (s, 1H), 7.28 (d, J=8.0 Hz, 2H), 7.16 (d, J=8.0 Hz, 2H), 7.05-6.94 (m, 1H), 4.21 (d, J=2.0 Hz, 2H), 3.46 (s, 2H), 2.54 (s, 1H), 2.20 (d, J=9.4 Hz, 2H), 1.90 (d, J=6.4 Hz, 2H), 1.76-1.69 (m, 2H). LCMS: Rt=1.09 min, m/z=408.0 (M+H).

### Example 32: 2-oxo-5-(4-((3-(pyridin-3-yl)morpholino)methyl)phenyl)-6-(trifluoromethyl)-1,2-dihydropyridine-3-carboxamide (32)

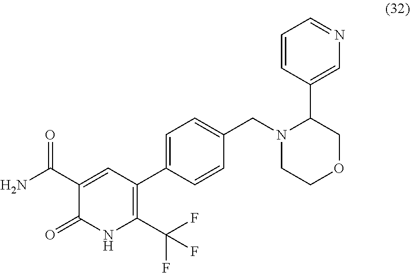 [SMILES: NC(=O)c1cc(-c2ccc(CN3CCOCC3c3cccnc3)cc2)c(C(F)(F)F)[nH]c1=O]

2-oxo-5-(4-((3-(pyridin-3-yl)morpholino)methyl)phenyl)-6-(trifluoromethyl)-1,2-dihydropyridine-3-carboxamide (32) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with 3-(pyridin-3-yl)morpholine. ¹H NMR (400 MHz, DMSO) δ=8.71 (s, 1H), 8.54-8.53 (m, 1H), 8.16 (s, 1H), 7.96-7.94 (m, 1H), 7.45-7.42 (m, 1H), 7.33-7.24 (m, 4H), 3.84-3.82 (m, 1H), 3.73-3.71 (m, 1H), 3.65-3.56 (m, 2H), 3.54-3.48 (m, 1H), 3.37 (br. s., 1H), 3.07-3.00 (m, 1H), 2.74-2.66 (m, 1H), 2.33-2.26 (m, 1H). LCMS: Rt=0.70 min, m/z=459.1 (M+H).

### Example 33: 5-(4-(1-morpholinoethyl)phenyl)-2-oxo-6-(trifluoromethyl)-1,2-dihydropyridine-3-carboxamide (33)

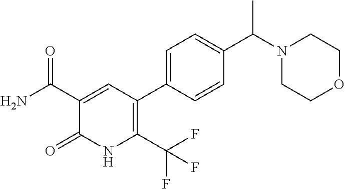 [SMILES: CC(c1ccc(-c2cc(C(N)=O)c(=O)[nH]c2C(F)(F)F)cc1)N1CCOCC1]

5-(4-(1-morpholinoethyl)phenyl)-2-oxo-6-(trifluoromethyl)-1,2-dihydropyridine-3-carboxamide (33) was made using a procedure similar to that used to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 1-(4-(4,4,5,5-tetramethyl-1,3,2-dioxaborolan-2-yl)phenyl)ethan-1-one, and 3-methylmorpholine was replaced with morpholine. ¹H NMR (400 MHz, DMSO) δ=9.15 (s, 1H), 8.15 (s, 1H), 7.82 (s, 1H), 7.35 (d, J=8.0 Hz, 1H), 7.25 (d, J=8.0 Hz, 1H), 3.75-3.55 (m, 5H), 2.40-2.30 (m, 2H), 2.29-2.26 (m., 2H), 1.31-1.25 (m, 3H). LCMS: Rt=0.53 min, m/z=396.3 (M+H).

### Example 34: 5-(4-((1,4-oxazepan-4-yl)methyl)phenyl)-2-oxo-6-(trifluoromethyl)-1,2-dihydropyridine-3-carboxamide (34)

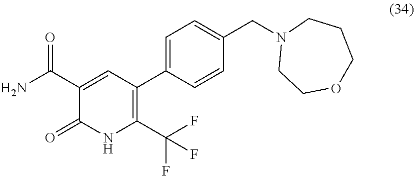 [SMILES: NC(=O)c1cc(-c2ccc(CN3CCCOCC3)cc2)c(C(F)(F)F)[nH]c1=O]

5-(4-((1,4-oxazepan-4-yl)methyl)phenyl)-2-oxo-6-(trifluoromethyl)-1,2-dihydropyridine-3-carboxamide (34) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with 1,4-oxazepane. ¹H NMR (400 MHz, DMSO) δ=10.24 (br, s, 1H), 7.92 (s, 1H), 7.34 (d, J=8.0 Hz, 2H), 7.25 (br. s., 1H), 7.20 (d, J=8.0 Hz, 2H), 3.71 (m, 2H), 3.67 (s, 2H), 3.63-3.61 (m, 2H), 2.66-2.62 (m, 4H), 1.84-1.81 (m, 2H). LCMS: Rt=1.00 min, m/z=396.3 (M+H).

### Example 35: 5-(4-(((2S,6R)-2,6-dimethylmorpholino)methyl)phenyl)-2-oxo-6-(trifluoromethyl)-1,2-dihydropyridine-3-carboxamide (35)

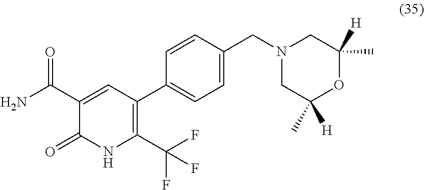 [SMILES: C[C@@H]1CN(Cc2ccc(-c3cc(C(N)=O)c(=O)[nH]c3C(F)(F)F)cc2)C[C@H](C)O1]

5-(4-(((2S,6R)-2,6-dimethylmorpholino)methyl)phenyl)-2-oxo-6-(trifluoromethyl)-1,2-dihydropyridine-3-carboxamide (35) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with (2S,6R)-2,6-dimethylmorpholine. ¹H NMR (400 MHz, DMSO) δ=8.82-8.68 (m, 1H), 8.26 (s, 1H), 8.05 (s, 1H), 7.43-7.36 (m, 2H), 7.34-7.24 (m, 2H), 3.61-3.59 (m, 2H), 3.53 (s, 2H), 2.71 (d, J=10.4 Hz, 2H), 1.71 (t, J=10.7 Hz, 2H), 1.04 (d, J=6.3 Hz, 6H). LCMS: Rt=0.98 min, m/z=410.2 (M+H).

### Example 36: 5-(3-fluoro-4-(morpholinomethyl)phenyl)-2-oxo-6-(trifluoromethyl)-1,2-dihydropyridine-3-carboxamide (36)

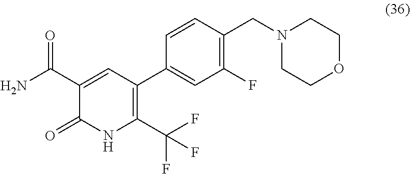 [SMILES: NC(=O)c1cc(-c2ccc(CN3CCOCC3)c(F)c2)c(C(F)(F)F)[nH]c1=O]

5-(3-fluoro-4-(morpholinomethyl)phenyl)-2-oxo-6-(trifluoromethyl)-1,2-dihydropyridine-3-carboxamide (36) was made using a procedure similar to that used to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 2-fluoro-4-(4,4,5,5-tetramethyl-1,3,2-dioxaborolan-2-yl)benzaldehyde, and 3-methylmorpholine was replaced with morpholine. ¹H NMR (400 MHz, DMSO) δ=8.39 (s, 1H), 8.30 (s, 1H), 8.18 (s, 1H), 7.68 (t, J=8.0 Hz, 1H), 7.42 (d, J=8.0 Hz, 1H), 7.33 (d, J=8.0 Hz, 1H), 4.40 (s., 2H), 3.79 (m., 5H), 3.19 (bs., 3H). LCMS: Rt=0.59 min, m/z=400.3 (M+H).

### Example 37: 5-(4-(1-hydroxyethyl)phenyl)-2-oxo-6-(trifluoromethyl)-1,2-dihydropyridine-3-carboxamide (37)

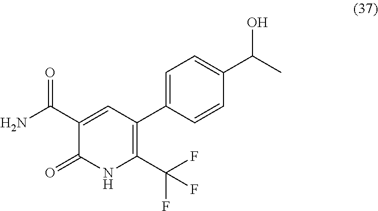 [SMILES: CC(O)c1ccc(-c2cc(C(N)=O)c(=O)[nH]c2C(F)(F)F)cc1]

5-(4-(1-hydroxyethyl)phenyl)-2-oxo-6-(trifluoromethyl)-1,2-dihydropyridine-3-carboxamide (37) was made using a procedure similar to that used in step 1 in the procedure to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 1-(4-(4,4,5,5-tetramethyl-1,3,2-dioxaborolan-2-yl)phenyl)ethan-1-ol. ¹H NMR (400 MHz, DMSO) δ=8.47 (s, 1H), 8.30 (s, 1H), 8.18 (s, 1H), 7.44 (d, J=8.0 Hz, 2H), 7.31 (d, J=8.0 Hz, 2H), 5.26 (s, 1H), 4.79 (d, J=6.4 Hz, 1H), 1.37 (d, J=6.4 Hz, 3H). LCMS: Rt=0.64 min, m/z=327.0 (M+H).

### Example 38: 5-(4-((3-methoxyazetidin-1-yl)methyl)phenyl)-2-oxo-6-(trifluoromethyl)-1,2-dihydropyridine-3-carboxamide (38)

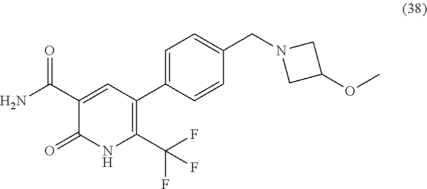 [SMILES: COC1CN(Cc2ccc(-c3cc(C(N)=O)c(=O)[nH]c3C(F)(F)F)cc2)C1]

5-(4-((3-methoxyazetidin-1-yl)methyl)phenyl)-2-oxo-6-(trifluoromethyl)-1,2-dihydropyridine-3-carboxamide (38) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with 3-methoxyazetidine. ¹H NMR (400 MHz, DMSO) δ=9.42 (s, 1H), 8.09 (s, 1H), 7.69 (s, 1H), 7.39 (d, J=7.8 Hz, 2H), 7.27 (d, J=7.8 Hz, 2H), 4.12-4.06 (m, 1H), 3.90 (s, 2H), 3.77 (s, 2H), 3.28-3.24 (m, 2H), 3.20-3.17. LCMS: Rt=0.48 min, m/z=382.1 (M+H).

### Example 39: 5-(4-((2,2-dimethylmorpholino)methyl)phenyl)-2-oxo-6-(trifluoromethyl)-1,2-dihydropyridine-3-carboxamide (39)

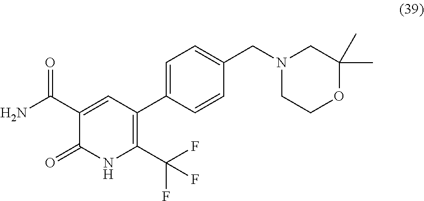 [SMILES: CC1(C)CN(Cc2ccc(-c3cc(C(N)=O)c(=O)[nH]c3C(F)(F)F)cc2)CCO1]

5-(4-((2,2-dimethylmorpholino)methyl)phenyl)-2-oxo-6-(trifluoromethyl)-1,2-dihydropyridine-3-carboxamide (39) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with 2,2-dimethylmorpholine. ¹H NMR (400 MHz, DMSO) δ=8.43 (s, 1H), 8.31 (s, 1H), 8.18 (s, 1H), 7.62 (d, J=8.0 Hz, 2H), 7.47 (d, J=8.0 Hz, 2H), 4.38 (br. s, 4H), 3.17-2.91 (m, 4H), 1.26 (br. s, 6H). LCMS: Rt=0.88 min, m/z=410.2 (M+H).

### Example 40: (S)-5-(4-((3-methoxypyrrolidin-1-yl)methyl)phenyl)-2-oxo-6-(trifluoromethyl)-1,2-dihydropyridine-3-carboxamide (40)

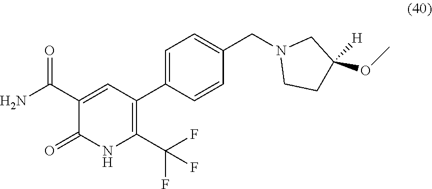 [SMILES: CO[C@H]1CCN(Cc2ccc(-c3cc(C(N)=O)c(=O)[nH]c3C(F)(F)F)cc2)C1]

(S)-5-(4-((3-methoxypyrrolidin-1-yl)methyl)phenyl)-2-oxo-6-(trifluoromethyl)-1,2-dihydropyridine-3-carboxamide (40) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with (S)-3-methoxypyrrolidine. ¹H NMR (400 MHz, DMSO) δ=9.64-9.33 (m, 1H), 8.09 (s, 1H), 7.66 (s, 1H), 7.38 (d, J=8.0 Hz, 2H), 7.26 (d, J=7.9 Hz, 2H), 3.99-3.88 (m, 1H), 3.74 (s, 2H), 3.17 (s, 3H), 2.90-2.78 (m, 1H), 2.75-2.67 (m, 1H), 2.60 (d, J=7.9 Hz, 2H), 2.08-1.99 (m, 1H), 1.81-1.66 (m, 1H). LCMS: Rt=0.52 min, m/z=396.1 (M+H).

### Example 41: (R)-5-(4-((3-methoxypyrrolidin-1-yl)methyl)phenyl)-2-oxo-6-(trifluoromethyl)-1,2-dihydropyridine-3-carboxamide (41)

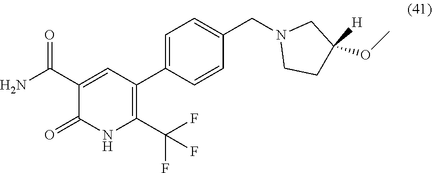 [SMILES: CO[C@@H]1CCN(Cc2ccc(-c3cc(C(N)=O)c(=O)[nH]c3C(F)(F)F)cc2)C1]

(R)-5-(4-((3-methoxypyrrolidin-1-yl)methyl)phenyl)-2-oxo-6-(trifluoromethyl)-1,2-dihydropyridine-3-carboxamide (41) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with (R)-3-methoxypyrrolidine. ¹H NMR (400 MHz, DMSO) δ=10.59 (s, 1H), 7.85 (s, 1H), 7.29 (d, J=8.0 Hz, 2H), 7.18 (d, J=7.6 Hz, 2H), 7.08 (br. s., 1H), 3.90-3.89 (m, 1H), 3.33 (s, 2H), 2.52 (s, 3H), 2.51-2.50 (m, 1H), 2.50-2.48 (m, 1H), 2.48-2.47 (m, 2H), 2.44-2.00 (m, 1H), 1.99-1.66 (m, 1H). LCMS: Rt=0.51 min, m/z=396.2 (M+H).

### Example 42: 5-(2-fluoro-4-(morpholinomethyl)phenyl)-2-oxo-6-(trifluoromethyl)-1,2-dihydropyridine-3-carboxamide (42)

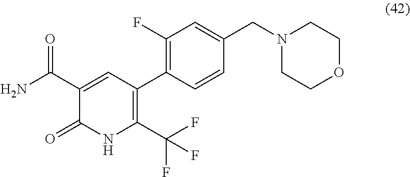 [SMILES: NC(=O)c1cc(-c2ccc(CN3CCOCC3)cc2F)c(C(F)(F)F)[nH]c1=O]

5-(2-fluoro-4-(morpholinomethyl)phenyl)-2-oxo-6-(trifluoromethyl)-1,2-dihydropyridine-3-carboxamide (42) was made using a procedure similar to that used to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 3-fluoro-4-(4,4,5,5-tetramethyl-1,3,2-dioxaborolan-2-yl)benzaldehyde, and 3-methylmorpholine was replaced with morpholine. ¹H NMR (400 MHz, DMSO) δ=8.33 (s, 1H), 8.29 (s, 1H), 8.22-8.10 (m, 1H), 7.63-7.40 (m, 3H), 4.34 (s, 2H), 3.79 (s, 4H), 3.13 (s, 5H). LCMS: Rt=0.49 min, m/z=400.3 (M+H).

### Example 43: 5-(3-methyl-4-(morpholinomethyl)phenyl)-2-oxo-6-(trifluoromethyl)-1,2-dihydropyridine-3-carboxamide (43)

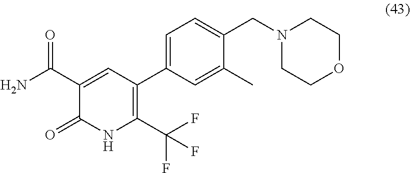 [SMILES: Cc1cc(-c2cc(C(N)=O)c(=O)[nH]c2C(F)(F)F)ccc1CN1CCOCC1]

5-(3-methyl-4-(morpholinomethyl)phenyl)-2-oxo-6-(trifluoromethyl)-1,2-dihydropyridine-3-carboxamide (43) was made using a procedure similar to that used to make 5-(4-((3-methylmorpholino)methylphenyl-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 2-methyl-4-(4,4,5,5-tetramethyl-1,3,2-dioxaborolan-2-yl)benzaldehyde, and 3-methylmorpholine was replaced with morpholine. ¹H NMR (400 MHz, DMSO) δ=8.40 (s, 1H), 8.27 (s, 1H), 8.16 (s, 1H), 7.55 (d, J=8.0 Hz, 1H), 7.36-7.23 (m, 2), 4.36 (s, 2H), 4.01-3.66 (m, 4H), 3.22 (s, 4H), 2.46 (s, 3H). LCMS: Rt=0.51 min, m/z=396.1 (M+H).

### Example 44: 2-oxo-5-(4-(piperidin-1-ylmethyl)phenyl)-6-(trifluoromethyl)-1,2-dihydropyridine-3-carboxamide (44)

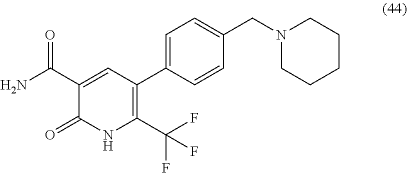 [SMILES: NC(=O)c1cc(-c2ccc(CN3CCCCC3)cc2)c(C(F)(F)F)[nH]c1=O]

2-oxo-5-(4-(piperidin-1-ylmethyl)phenyl)-6-(trifluoromethyl)-1,2-dihydropyridine-3-carboxamide (44) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with piperidine. ¹H NMR (400 MHz, DMSO) δ=7.87 (s, 1H), 7.27 (d, J=8.0, 2H), 7.16 (d, J=8.0, 2H), 3.52 (s, 2H), 2.49 (m, 4H), 1.53 (m, 4H), 1.40 (m, 2H). LCMS: Rt=1.03 min, m/z=380.2 (M+H).

### Example 45: 5-(4-((dimethylamino)methyl)phenyl)-2-oxo-6-(trifluoromethyl)-1,2-dihydropyridine-3-carboxamide (45)

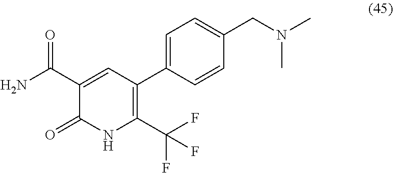 [SMILES: CN(C)Cc1ccc(-c2cc(C(N)=O)c(=O)[nH]c2C(F)(F)F)cc1]

5-(4-((dimethylamino)methyl)phenyl)-2-oxo-6-(trifluoromethyl)-1,2-dihydropyridine-3-carboxamide (45) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with dimethylamine. ¹H NMR (400 MHz, DMSO) δ=10.59 (s, 1H), 7.85 (s, 1H), 7.28-7.16 (m, 4H), 7.06 (s, 1H), 3.45 (s, 2H), 2.19 (s, 6H). LCMS: Rt=0.40 min, m/z=340.2 (M+H).

### Example 46: 2-oxo-5-(4-((3-oxomorpholino)methyl)phenyl)-6-(trifluoromethyl)-1,2-dihydropyridine-3-carboxamide (46)

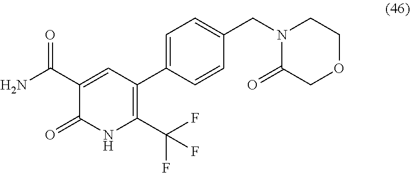 [SMILES: NC(=O)c1cc(-c2ccc(CN3CCOCC3=O)cc2)c(C(F)(F)F)[nH]c1=O]

2-oxo-5-(4-((3-oxomorpholino)methyl)phenyl)-6-(trifluoromethyl)-1,2-dihydropyridine-3-carboxamide (46) was made using a procedure similar to that used in step 1 in the procedure to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 4-(4-(4,4,5,5-tetramethyl-1,3,2-dioxaborolan-2-yl)benzyl)morpholin-3-one. ¹H NMR (400 MHz, DMSO) δ=8.46 (s, 1H), 8.31 (s, 1H), 8.19 (s, 1H), 7.36 (s, 4H), 4.62 (s, 2H), 4.15 (s, 2H), 3.86 (t, J=5.1 Hz, 2H), 3.32-3.29 (m, 2H). LCMS: Rt=0.59 min, m/z=396.0 (M+H).

### 4-(4-(4,4,5,5-tetramethyl-1,3,2-dioxaborolan-2-yl)benzyl)morpholin-3-one was Obtained Using the Following Procedure

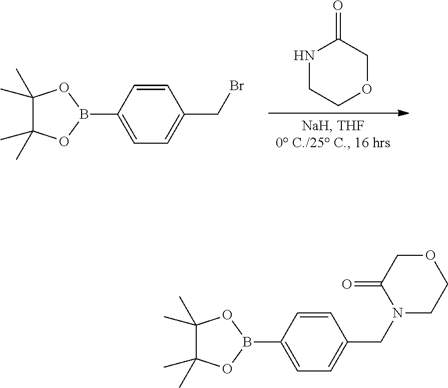

To a solution of morpholin-3-one (40 mg, 0.78 mmol) in THF (2 mL) was added NaH (12 mg, 0.98 mmol) at 0° C. Then the mixture was stirred at 25° C. for 1 hr. 2-(4-(bromomethyl)phenyl))-4,4,5,5-tetramethyl-1,3,2-dioxaborolane (200 mg, 0.68 mmol) was then added and the mixture was stirred at 25° C. for 15 hrs. The reaction mixture was poured into water (10 mL) and the resulting mixture extracted with EA (10 mL×2). The combined organic layers were dried over anhydrous Na₂SO₄, filtered and concentrated to give 4-(4-(4,4,5,5-tetramethyl-1,3,2-dioxaborolan-2-yl)benzyl)morpholin-3-one. The crude product was used directly without purification. TLC:PE:EA=1:1, Rf=0.2. LCMS: Rt=0.78 min, m/z=318.1 (M+H).

### Example 47: 5-(4-((1,1-dioxidothiomorpholino)methyl)phenyl)-2-oxo-6-(trifluoromethyl)-1,2-dihydropyridine-3-carboxamide (47)

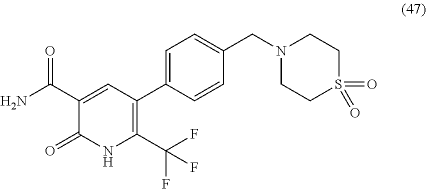 [SMILES: NC(=O)c1cc(-c2ccc(CN3CCS(=O)(=O)CC3)cc2)c(C(F)(F)F)[nH]c1=O]

5-(4-((1,1-dioxidothiomorpholino)methyl)phenyl)-2-oxo-6-(trifluoromethyl)-1,2-dihydropyridine-3-carboxamide (47) was made using a procedure similar to that used to make 5-(4-((3-methylmorpholino)methyl)phenyl)-2-oxo-6-(trifluoromethyl)-1,2-dihydropyridine-3-carboxamide (27), except 3-methylmorpholine was replaced with thiomorpholine 1,1-dioxide. ¹H NMR (400 MHz, DMSO) δ=9.62 (br, s, 1H), 8.06 (s, 1H), 7.59 (br, s, 1H), 7.38 (d, J=8.0 Hz, 2H), 7.27 (d, J=8.0 Hz, 2H), 3.71 (s, 2H), 3.14 (m, 4H), 2.91 (m, 4H). LCMS: Rt=1.1 min, m/z=430.1 (M+H).

### Example 48: 5-(4-(morpholino(oxazol-5-yl)methyl)phenyl)-2-oxo-6-(trifluoromethyl)-1,2-dihydropyridine-3-carboxamide (48)

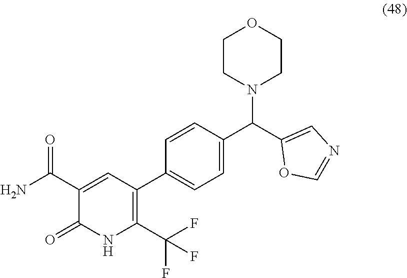 [SMILES: NC(=O)c1cc(-c2ccc(C(c3cnco3)N3CCOCC3)cc2)c(C(F)(F)F)[nH]c1=O]

5-(4-(morpholino(oxazol-5-yl)methyl)phenyl)-2-oxo-6-(trifluoromethyl)-1,2-dihydropyridine-3-carboxamide (48) was made using a procedure similar to that used in step 1 in the procedure to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 4-(oxazol-5-yl(4-(4,4,5,5-tetramethyl-1,3,2-dioxaborolan-2-yl)phenyl)methyl)morpholine. 1H NMR (400 MHz, DMSO) δ=10.89-10.74 (m, 1H), 8.33 (s, 1H), 7.79 (s, 1H), 7.40 (d, J=8.0 Hz, 2H), 7.21 (d, J=8.0 Hz, 2H), 7.17 (s, 1H), 6.98 (d, J=6.1 Hz, 1H), 4.72 (s, 1H), 3.59 (t, J=4.3 Hz, 4H), 2.41-2.26 (m, 4H). LCMS: Rt=0.78 min, m/z=449.2 (M+H).

### 4-(oxazol-5-yl(4-(4,4,5,5-tetramethyl-1,3,2-dioxaborolan-2-yl)phenyl)methyl)morpholine was Obtained Using the Following Procedure

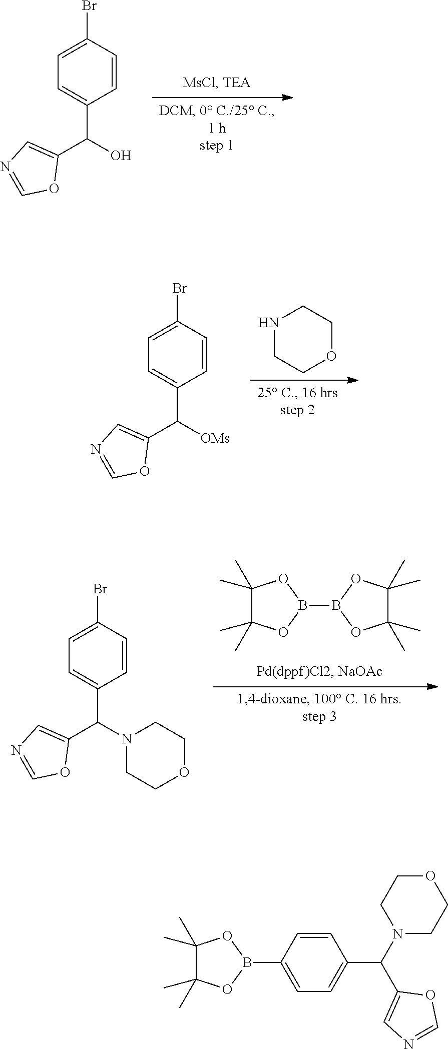

Step 1: To a solution of (4-bromophenyl)(oxazol-5-yl)methanol (Science of Synthesis (2002), 11, 383479) (400 mg, 1.9 mmol, TEA (500 mg, 3.8 mmol) in DCM (4 mL) was added MsCl (200 mg, 2.3 mmol) at 0° C. Then the mixture was stirred at 25° C. for 1 hr to give crude (4-bromophenyl)(oxazol-5-yl)methyl methanesulfonate which was used directly in the next step. TLC:PE:EA=1:1 Rf=0.7.

Step 2: A solution of (4-bromophenyl)(oxazol-5-yl)methyl methanesulfonate (400 mg, 1.2 mmol) in morpholine (4 mL) was stirred at 25° C. for 16 hr. The reaction mixture was poured into water (10 mL) and the resulting mixture extracted with EA (10 mL×2). The combined organic layers were dried over anhydrous Na₂SO₄, filtered and concentrated to give crude product which was purified by column chromatography (PE:EA=3:1˜1:1) to give 4-((4-bromophenyl)(oxazol-5-yl)methyl)morpholine. TLC:PE:EA=1:1 Rf=0.3. ¹H NMR: (CDCl3, 400 MHz), δ: 7.86 (s, 1H), 7.50 (d, J=8.4 Hz, 2H), 7.35 (d, J=8.4 Hz, 2H), 7.02 (s, 1H), 4.50 (s, 1H), 3.73 (t, J=4.6 Hz, 4H), 2.41 (m, 4H).

Step 3: To a solution of 4-((4-bromophenyl)(oxazol-5-yl)methyl)morpholine (200 mg, 1.0 mmol, 4,4,4′,4′,5,5,5′,5′-octamethyl-2,2′-bi(1,3,2-dioxaborolane) (500 mg, 2.0 mmol, NaOAc (240 mg, 3.0 mmol) in dioxane (2 mL) was added Pd(dppf)Cl₂ (26 mg, 0.05 mmol), the mixture was stirred at 100° C. for 16 hrs. The mixture was diluted with H₂O (10 mL), extracted by EA (5.0 mL×3), dried over Na₂SO₄, filtered and concentrated in vacuo to give 4-(oxazol-5-yl(4-(4,4,5,5-tetramethyl-1,3,2-dioxaborolan-2-yl)phenyl)methyl)morpholine. LCMS: Rt=1.20 min, m/z=371.1 (M+H).

### Example 49: 5-(4-(methylsulfonamidomethyl)phenyl)-2-oxo-6-(trifluoromethyl)-1,2-dihydropyridine-3-carboxamide (49)

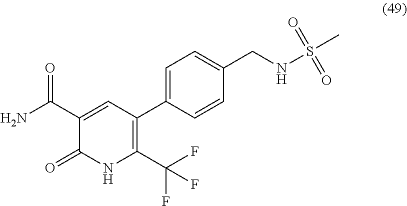 [SMILES: CS(=O)(=O)NCc1ccc(-c2cc(C(N)=O)c(=O)[nH]c2C(F)(F)F)cc1]

5-(4-(methylsulfonamidomethyl)phenyl)-2-oxo-6-(trifluoromethyl)-1,2-dihydropyridine-3-carboxamide (49) was made using a procedure similar to that used in step 1 in the procedure to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with N-(4-(4,4,5,5-tetramethyl-1,3,2-dioxaborolan-2-yl)benzyl)methanesulfonamide. ¹H NMR (400 MHz, DMSO) δ=8.46 (br, s, 1H), 8.29 (s, 1H), 8.18 (br, s, 1H), 7.64 (t, 1H), 7.46 (d, J=8.2 Hz, 2H), 7.36 (m, 2H), 4.24 (d, J=6.4 Hz, 2H), 2.88 (s, 3H). LCMS: Rt=0.60 min, m/z=390.0 (M+H).

### Example 50: 5-(4-((4-hydroxytetrahydro-2H-pyran-4-yl)methyl)phenyl)-2-oxo-6-(trifluoromethyl)-1,2-dihydropyridine-3-carboxamide (50)

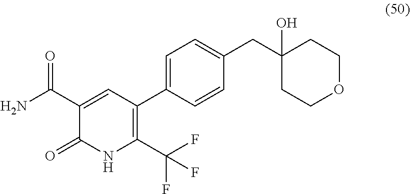 [SMILES: NC(=O)c1cc(-c2ccc(CC3(O)CCOCC3)cc2)c(C(F)(F)F)[nH]c1=O]

5-(4-((4-hydroxytetrahydro-2H-pyran-4-yl)methyl)phenyl)-2-oxo-6-(trifluoromethyl)-1,2-dihydropyridine-3-carboxamide (50) was made using a procedure similar to that used in step 1 in the procedure to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 4-(4-(4,4,5,5-tetramethyl-1,3,2-dioxaborolan-2-yl)benzyl)tetrahydro-2H-pyran-4-ol. TLC:PE:EA=1:1 Rf=0.1. LCMS: Rt=0.622 min, [M+H]⁺=397.1. ¹H NMR (400 MHz, DMSO) δ=9.56-10.13 (m, 1H), 8.02 (s, 1H), 7.44-7.56 (m, 1H), 7.25 (d, J=7.6 Hz, 2H), 7.07-7.21 (m, 2H), 4.35-4.50 (m, 1H), 3.60 (d, J=6.6 Hz, 4H), 2.73 (s, 2H), 1.48-1.66 (m, 2H), 1.32 (d, J=13.2 Hz, 2H).

4-(4-(4,4,5,5-tetramethyl-1,3,2-dioxaborolan-2-yl)benzyl)tetrahydro-2H-pyran-4-ol was obtained using a method similar to step 3 in the preparation of 4-(oxazol-5-yl(4-(4,4,5,5-tetramethyl-1,3,2-dioxaborolan-2-yl)phenyl))methyl)morpholine except 4-((4-bromophenyl)(oxazol-5-yl)methyl)morpholine was replaced with 4-(4-bromobenzyl)tetrahydro-2H-pyran-4-ol.

### 4-(4-bromobenzyl)tetrahydro-2H-pyran-4-ol was Prepared Using the Following Procedure

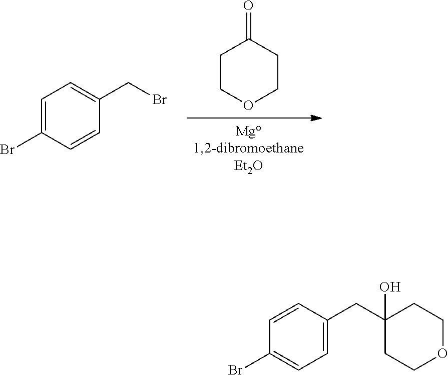

To a mixture of Mg (1 g, 41 mmol), 1,2-dibromoethane (10 mg, 0.1 mmol) in diethyl ether (20 mL) was added 1-bromo-4-(bromomethyl)benzene (1 g, 1 mmol) drop-wise. The mixture was stirred at 0° C. for 1 hr. Then the mixture was added tetrahydro-4H-pyran-4-one (400 mg, 1.2 mmol) at 0° C. Then the mixture was stirred at 25° C. for 15 hr. The reaction mixture was poured into water (20 mL) and the resulting mixture extracted with EA (20 mL×2). The combined organic layers were dried over anhydrous Na₂SO₄, filtered and concentrated to get the crude product. The crude product was purified by column chromatography (PE:EA=5:1˜1:1) to give 4-(4-bromobenzyl)tetrahydro-2H-pyran-4-ol. TLC:PE:EA=1:1 Rf=0.5. LCMS: Rt=0.69 min, [M+H]⁺=253.0, ¹H NMR (400 MHz, DMSO) δ=7.50-7.44 (m, 2H), 7.11 (d, J=8.4 Hz, 2H), 3.79-3.72 (m, 4H), 2.75 (s, 2H), 1.81-1.72 (m, 2H), 1.52-1.39 (m, 2H).

### Example 51: 5-(4-((4-fluorotetrahydro-2H-pyran-4-yl)methyl)phenyl)-2-oxo-6-(trifluoromethyl)-1,2-dihydropyridine-3-carboxamide (51)

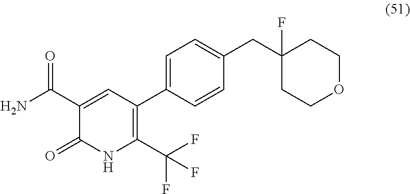 [SMILES: NC(=O)c1cc(-c2ccc(CC3(F)CCOCC3)cc2)c(C(F)(F)F)[nH]c1=O]

5-(4-((4-fluorotetrahydro-2H-pyran-4-yl)methyl)phenyl)-2-oxo-6-(trifluoromethyl)-1,2-dihydropyridine-3-carboxamide (51) was made using a procedure similar to that used in step 1 in the procedure to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 2-(4-((4-fluorotetrahydro-2H-pyran-4-yl)methyl)phenyl-4,4,5,5-tetramethyl-1,3,2-dioxaborolane. TLC:PE:EA=1:1 Rf=0.1. LCMS: Rt=0.775 min, [M+H]⁺=399.0. ¹H NMR (400 MHz, DMSO) δ=14.23-13.31 (m, 1H), 8.49 (s, 1H), 8.32 (s, 1H), 8.22 (s, 1H), 7.42-7.25 (m, 4H), 3.74 (m, 2H), 3.56-3.51 (m, 2H), 3.11-2.93 (m, 2H), 1.90-1.65 (m, 2H), 1.65-1.52 (m, 2H).

2-(4-((4-fluorotetrahydro-2H-pyran-4-ylmethyl)phenyl)-4,4,5,5-tetramethyl-1,3,2-dioxaborolane was obtained using a method similar to step 3 in the preparation of 4-(oxazol-5-yl(4-(4,4,5,5-tetramethyl-1,3,2-dioxaborolan-2-yl)phenyl)methyl)morpholine except 4-((4-bromophenyl)(oxazol-5-yl)methyl)morpholine was replaced with 4-(4-bromobenzyl)-4-fluorotetrahydro-2H-pyran.

### 4-(4-bromobenzyl)-4-fluorotetrahydro-2H-pyran was Prepared Using the Following Procedure

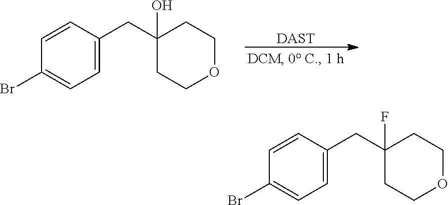

To a solution of 4-(4-bromobenzyl)tetrahydro-2H-pyran-4-ol (500 mg, 2 mmol) in DCM (5 mL) was added DAST (300 mg, 2.2 mmol) at 0° C. Then the mixture was stirred at 0° C. for 1 hr. The reaction mixture was poured into water (10 mL) and the resulting mixture extracted with EA (10 mL×2). The combined organic layers were dried over anhydrous Na2SO4, filtered and concentrated to get the crude product. The crude product was purified by column chromatography (PE:EA=10:1˜3:1) to get 4-(4-bromobenzyl)-4-fluorotetrahydro-2H-pyran (300 mg, 1.2 mmol, 60% yield) as colorless solid. TLC:PE:EA=1:1 Rf=0.6.

### Example 52: 2-oxo-5-(4-((tetrahydro-2H-pyran-4-yl)methyl)phenyl)-6-(trifluoromethyl)-1,2-dihydropyridine-3-carboxamide 52

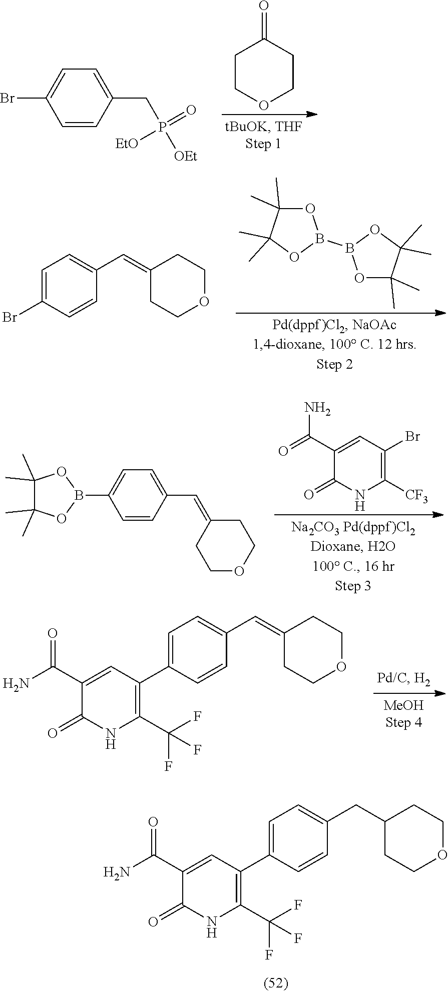

Step 1. To a solution of diethyl (4-bromobenzyl)phosphonate (500 mg, 1 mmol, and tetrahydro-4H-pyran-4-one (200 mg, 1.2 mmol in THF (5 mL) was added tBuOK (300 mg, 2 mmol). Then the mixture was stirred at 25° C. for 16 hr. The reaction mixture was poured into water (10 mL). The resulting mixture was extracted with EA (10 mL×2). The combined organic layers were dried over anhydrous Na₂SO₄, filtered and concentrated to get the crude product. The crude product was purified column chromatography (5:1˜3:1) to give 4-(4-bromobenzylidene)tetrahydro-2H-pyran. TLC:PE:EA=1:1 Rf=0.4. LCMS: Rt=1.00 min, [M+H]⁺=252.9. ¹H NMR (400 MHz, CDCl3) δ=7.44 (d, J=8.4 Hz, 1H), 7.07 (d, J=8.4 Hz, 1H), 6.26 (s, 1H), 3.79 (t, J=5.4 Hz, 2H), 3.67 (t, J=5.4 Hz, 2H), 2.54-2.46 (m, 2H), 2.44-2.35 (m, 2H).

Step 2: To a solution of 4-(4-bromobenzylidene)tetrahydro-2H-pyran (300 mg, 1.0 mmol), 4,4,4′,4′,5,5,5′,5′-octamethyl-2,2′-bi(1,3,2-dioxaborolane) (500 mg, 2.0 mmol), NaOAc (240 mg, 3.0 mmol) in dioxane (2 mL) was added Pd(dppf)Cl₂ (26 mg, 0.05 mmol), the mixture was stirred at 100° C. for 16 hr to give which 4,4,5,5-tetramethyl-2-(4-((tetrahydro-4H-pyran-4-ylidene)methyl)phenyl)-1,3,2-dioxaborolane was used directly in the next step. LCMS: Rt=1.12 min, [M+H]⁺=301.1.

Step 3: To a solution of 5-bromo-2-oxo-6-(trifluoromethyl)-1,2-dihydropyridine-3-carboxamide (200 mg, 0.70 mmol), 4,4,5,5-tetramethyl-2-(4-((tetrahydro-4H-pyran-4-ylidene)methyl)phenyl)-1,3,2-dioxaborolane (240 mg, 0.84 mmol), Na₂CO₃ (149 mg, 1.40 mmol) in dioxane:H₂O=9:1 (2 mL) was added Pd(dppf)Cl₂ (26 mg, 0.04 mmol). The mixture was then stirred at 100° C. for 16 hr. The reaction mixture was poured into water (10 mL) and extracted with EA (10 mL×2). The combined organic layers were dried over anhydrous Na₂SO₄, filtered and concentrated to give crude product. The crude product was purified by prep-HPLC (TFA) to give 2-oxo-5-(4-((tetrahydro-4H-pyran-4-ylidene)methyl)phenyl)-6-(trifluoromethyl)-1,2-dihydropyridine-3-carboxamide. TLC:PE:EA=1:1 Rf=0.1. LCMS: Rt=0.87 min, [M+H]⁺=379.1.

Step 4: To a solution of 2-oxo-5-(4-((tetrahydro-4H-pyran-4-ylidene)methyl)phenyl)-6-(trifluoromethyl)-1,2-dihydropyridine-3-carboxamide (50 mg, 0.13 mmol) in MeOH (2 mL) was added Pd/C (10 mg) and the mixture stirred at 25° C. under a balloon of hydrogen gas for 16 hrs. The reaction mixture was filtered and concentrated to give crude product. The crude product was purified by prep-HPLC (TFA) to give 2-oxo-5-(4-((tetrahydro-2H-pyran-4-yl)methyl)phenyl)-6-(trifluoromethyl)-1,2-dihydropyridine-3-carboxamide. TLC:PE:EA=1:1 Rf=0.3. LCMS: Rt=0.75 min, [M+H]⁺=381.0. ¹H NMR (400 MHz, DMSO) δ=9.90-9.31 (m, 1H), 8.09 (s, 1H), 7.79-7.53 (m, 1H), 7.30-7.05 (m, 4H), 3.82 (d, J=11.2 Hz, 2H), 3.27-3.21 (m, 2H), 2.58-2.53 (m, 2H), 1.76 (t, J=7.2 Hz, 1H), 1.49 (d, J=11.0 Hz, 2H), 1.26-1.20 (m, 2H).

### Example 53. 5-(4-(2-(1,4-dioxan-2-yl)-1,1-difluoroethyl)phenyl)-2-oxo-6-(trifluoromethyl)-1,2-dihydropyridine-3-carboxamide (53)

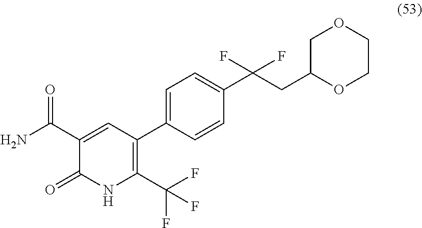 [SMILES: NC(=O)c1cc(-c2ccc(C(F)(F)CC3COCCO3)cc2)c(C(F)(F)F)[nH]c1=O]

5-(4-(2-(1,4-dioxan-2-yl)-1,1-difluoroethyl)phenyl)-2-oxo-6-(trifluoromethyl)-1,2-dihydropyridine-3-carboxamide (53) was made using a procedure similar to that used in step 1 in the procedure to make 5-(4-((3-methylmorpholino)methyl)phenyl)-2-oxo-6-(trifluoromethyl)-1,2-dihydropyridine-3-carboxamide (27), except 4-(4,4,5,5-tetramethyl-1,3,2-dioxaborolan-2-yl)benzaldehyde was replaced with 0.2-(4-(2-(1,4-dioxan-2-yl)-1,1-difluoroethyl)phenyl)-4,4,5,5-tetramethyl-1,3,2-dioxaborolane

2-(4-(2-(1,4-dioxan-2-yl)-1,1-difluoroethyl)phenyl)-4,4,5,5-tetramethyl-1,3,2-dioxaborolane was obtained using a method similar to step 3 in the preparation of 4-(oxazol-5-yl(4-(4,4,5,5-tetramethyl-1,3,2-dioxaborolan-2-yl)phenyl)methyl)morpholine except 4-((4-bromophenyl)(oxazol-5-yl)methyl)morpholine was replaced with 2-(2-(4-bromophenyl)-2,2-difluoroethyl)-1,4-dioxane.

### 2-(2-(4-bromophenyl)-2,2-difluoroethyl)-1,4-dioxane was Prepared Using the Following Procedure

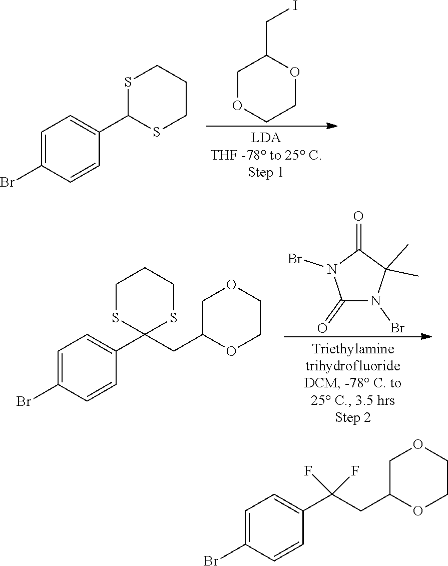 [SMILES: Brc1ccc(C2SCCCS2)cc1 ; ICC1COCCO1 ; Brc1ccc(C2(CC3COCCO3)SCCCS2)cc1 ; CC1(C)C(=O)N(Br)C(=O)N1Br ; FC(F)(CC1COCCO1)c1ccc(Br)cc1]

Step 1: 2-(4-bromophenyl)-1,3-dithiane (2.0 g, 7.27 mmol) in THF (30 ml) was cooled to −78° C. and LDA (4.36 mL, 10.9 mmol) was added dropwise. The mixture was allowed to warm to 0° C. and then added dropwise to a solution of 2-(iodomethyl)-1,4-dioxane (1.82 g, 7.99 mmol) in THF (30 mL) at 0° C. Then the mixture was allowed to warm to 25° C. and stirred for 3 h. The reaction mixture was poured into water (100 mL) and extracted with EA (100 mL×2). The combined organic phase was dried over anhydrous Na2SO4, filtered and concentrated to give crude product. The crude product was purified by silica gel column (PE/EA=20/1 to 5/1) to give 2-((2-(4-bromophenyl)-1,3-dithian-2-yl)methyl)-1,4-dioxane. TLC: PE/EA=5/1, Rf=0.60. LCMS: Rt=1.02, [M+H]⁺=375/377. ¹H NMR (400 MHz, CDCl3) δ=7.84 (d, J=8.0 Hz, 2H), 7.51 (d, J=8.0 Hz, 2H), 3.69-3.42 (m, 6H), 3.16 (m, 1H), 2.75-2.62 (m, 4H), 2.18 (m, 1H), 1.99-1.91 (m, 2H), 1.89 (m, 1H).

Step 2: Triethylamine trihydrofluoride (2.15 g, 13.32 mmol, Et₃N-3HF) was added to a mixture of 2-((2-(4-bromophenyl)-1,3-dithian-2-yl)methyl)-1,4-dioxane (1.0 g, 2.66 mmol) in DCM (50 ml) at −78° C. Then 1,3-dibromo-5,5-dimethylimidazolidine-2,4-dione (3.81 g, 13.32 mmol) in DCM (50 ml) was added in 30 min. The mixture was stirred for 3.5 h at 25° C. The solution was quenched with NaOH (1N, 50 mL) and extracted with DCM (50 ml×3). The separated organic layer was dried over Na₂SO₄, filtered and concentrated in vacuo. The crude product was purified by reversed phase column chromatography (FA) to afford 2-(2-(4-bromophenyl)-2,2-difluoroethyl)-1,4-dioxane. TLC: PE/EA=5:1, Rf=0.5. LCMS: Rt=5.979 min, M=306/308. ¹H NMR (400 MHz, CHLOROFORM-d) δ=7.59 (d, J=8.0 Hz, 2H), 7.37 (d, J=8.0 Hz, 2H), 3.84-3.55 (m, 6H), 3.34 (m, 1H), 2.44-2.28 (m, 1H), 2.23-2.06 (m, 1H).

### Example 54: 5-(4-((3-nitrobenzyl)oxy)phenyl)-2-oxo-6-(trifluoromethyl)-1,2-dihydropyridine-3-carboxamide (54)

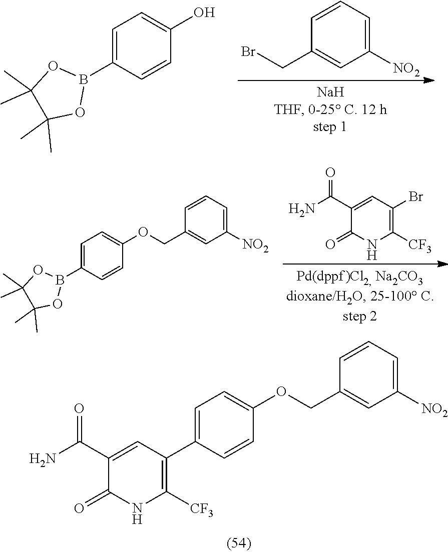 [SMILES: CC1(C)OB(c2ccc(O)cc2)OC1(C)C ; O=[N+]([O-])c1cccc(CBr)c1 ; CC1(C)OB(c2ccc(OCc3cccc([N+](=O)[O-])c3)cc2)OC1(C)C ; NC(=O)c1cc(Br)c(C(F)(F)F)[nH]c1=O ; NC(=O)c1cc(-c2ccc(OCc3cccc([N+](=O)[O-])c3)cc2)c(C(F)(F)F)[nH]c1=O]

Step 1: To a solution of 4-(4,4,5,5-tetramethyl-1,3,2-dioxaborolan-2-yl)phenol (3.0 g, 13.6 mmol) in THF (30 mL) was added NaH (1.1 g 27.2 mmol) at 0° C. and the mixture stirred at 0° C. for 0.5 hrs. 1-(bromomethyl)-3-nitrobenzene (3.5 g, 16.4 mmol) was then added to the above mixture at 25° C. The mixture was washed with H₂O (100 mL) and extracted with EA (100 mL×2). The combined organic layers were dried over anhydrous Na₂SO₄, filtered and concentrated to give crude 4,4,5,5-tetramethyl-2-(4-((3-nitrobenzyl)oxy)phenyl)-1,3,2-dioxaborolane. The residue was taken to the next step without purification. LCMS: Rt=0.99 min, m/z=356.2 (M+H).

Step 2: To a solution of 5-bromo-2-oxo-6-(trifluoromethyl)-1,2-dihydropyridine-3-carboxamide (4.0 g, 14.1 mmol) in dioxane (200 mL) and H₂O (20 mL) was added 4,4,5,5-tetramethyl-2-(4-((3-nitrobenzyl)oxy)phenyl)-1,3,2-dioxaborolane (5.0 g, 14.1 mmol), Na₂CO₃ (3.0 g, 28.2 mmol) and PdCl₂(dppf)-CH₂Cl₂ (500 mg, 0.7 mmol) at 25° C. under N₂. The mixture was stirred at 100° C. for 12 hrs. The mixture was washed with H₂O (100 mL) and extracted with EA (200 mL×2). The combined organic layers were dried over anhydrous Na₂SO₄, filtered, and concentrated. The residue was purified by column chromatography (PE/EA=1/1) to give 5-(4-((3-nitrobenzyl)oxy)phenyl)-2-oxo-6-(trifluoromethyl)-1,2-dihydropyridine-3-carboxamide (54). ¹H NMR (400 MHz, DMSO) δ=13.65 (br. s., 1H), 8.51-8.42 (m, 1H), 8.37-8.33 (m, 1H), 8.30-8.26 (m, 1H), 8.24-8.14 (m, 2H), 7.97-7.92 (m, 1H), 7.75-7.69 (m, 1H), 7.30 (d, J=8.5 Hz, 2H), 7.14 (d, J=8.7 Hz, 2H), 5.33 (s, 2H). LCMS: Rt=0.85 min, m/z=434.2 (M+H).

### Example 55: 5-(4-((1-acetylpiperidin-4-yl)methoxy)phenyl)-2-oxo-6-(trifluoromethyl)-1,2-dihydropyridine-3-carboxamide (55)

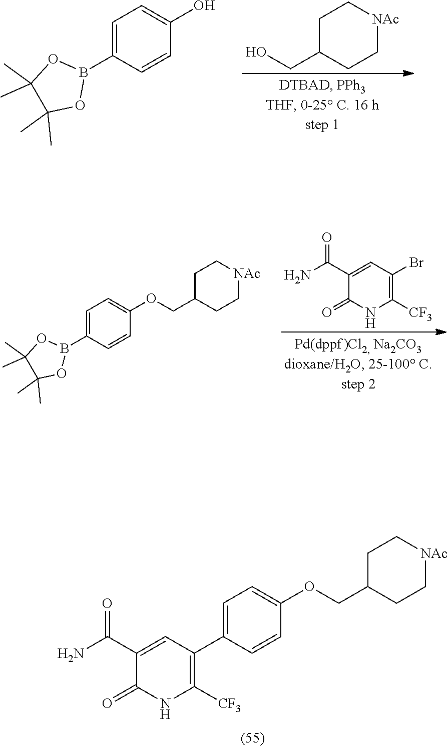

Step 1: To a solution of 1-(4-(hydroxymethyl)piperidin-1-yl)ethan-1-one (200 mg, 1.3 mmol), 4-(4,4,5,5-tetramethyl-1,3,2-dioxaborolan-2-yl)phenol (250 mg, 1.4 mmol), PPh₃ (600 mg, 2.6 mmol) in THF (5 mL) was added DTBAD (500 mg, 2.6 mmol) at 0° C. Then the mixture was stirred at 25° C. for 16 hrs. The reaction mixture was poured into water (20 ml) and then extracted with EA (20 mL×2). The combined organic layers were dried over anhydrous Na₂SO₄, filtered and concentrated to give crude 1-(4-((4-(4,4,5,5-tetramethyl-1,3,2-dioxaborolan-2-yl)phenoxy)methyl)piperidin-1-yl)ethan-1-one which was used directly in the next step. TLC: PE/EA=1:1, Rf=0.6. LCMS: Rt=1.60 min, m/z=360.2 (M+H).

Step 2: To a solution of 5-bromo-2-oxo-6-(trifluoromethyl)-1,2-dihydropyridine-3-carboxamide (200 mg, 0.70 mmol), 1-(4-((4-(4,4,5,5-tetramethyl-1,3,2-dioxaborolan-2-yl)phenoxy)methyl)piperidin-1-yl)ethan-1-one (320 mg, 0.84 mmol), Na₂CO₃ (149 mg, 1.40 mmol) in dioxane (2 mL) and H₂O (0.2 mL) was added PdCl₂(dppf)-CH₂Cl₂ (26 mg, 0.04 mmol) under N₂, and the mixture was stirred at 100° C. for 16 hrs. The reaction mixture was poured into water (10 mL) and extracted with ethyl acetate (10 mL×2). The combined organic layers were dried over anhydrous Na₂SO₄, filtered and concentrated to give crude product. The crude product was purified by Prep-HPLC under the acidic conditions (MeCN/water with TFA) to give 5-(4-((1-acetylpiperidin-4-yl)methoxy)phenyl)-2-oxo-6-(trifluoromethyl)-1,2-dihydropyridine-3-carboxamide (55). ¹H NMR (400 MHz, DMSO) δ=8.70-8.48 (m, 1H), 8.25 (s, 1H), 8.17-8.07 (m, 1H), 7.26 (d, J=8.6 Hz, 2H), 7.02 (d, J=8.8 Hz, 2H), 4.54-4.34 (m, 1H), 4.01-3.81 (m, 3H), 3.13-3.00 (m, 1H), 2.61-2.54 (m, 1H), 2.01 (s, 4H), 1.81 (s, 2H), 1.37-1.08 (m, 2H). LCMS: Rt=0.73 min, m/z=438.1 (M+H).

### Example 56: 2-oxo-5-(4-((tetrahydro-2H-pyran-4-yl)methoxy)phenyl)-6-(trifluoromethyl)-1,2-dihydropyridine-3-carboxamide (56)

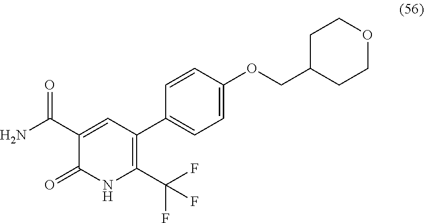 [SMILES: NC(=O)c1cc(-c2ccc(OCC3CCOCC3)cc2)c(C(F)(F)F)[nH]c1=O]

2-oxo-5-(4-((tetrahydro-2H-pyran-4-yl)methoxy)phenyl)-6-(trifluoromethyl)-1,2-dihydropyridine-3-carboxamide (56) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (tetrahydro-2H-pyran-4-yl)methanol. ¹H NMR (400 MHz, DMSO) δ=13.62 (s, 1H), 8.46 (s, 1H), 8.27 (s, 1H), 8.18 (s, 1H), 7.26 (d, J=8.0 Hz, 2H), 7.02 (d, J=8.0 Hz, 2H), 3.88 (d, J=8.0 Hz, 4H), 3.33-3.28 (m, 2H), 2.06-1.95 (m, 1H), 1.69 (m, 2H), 1.34 (m, 2H). LCMS: Rt=0.79 min, m/z=397.1 (M+H).

### Example 57: 5-(4-((3-aminobenzyl)oxy)phenyl)-2-oxo-6-(trifluoromethyl)-1,2-dihydropyridine-3-carboxamide (57)

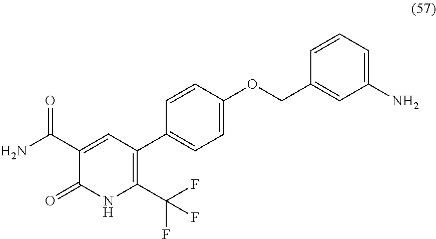 [SMILES: NC(=O)c1cc(-c2ccc(OCc3cccc(N)c3)cc2)c(C(F)(F)F)[nH]c1=O]

5-(4-((3-aminobenzyl)oxy)phenyl)-2-oxo-6-(trifluoromethyl)-1,2-dihydropyridine-3-carboxamide (57) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)aniline. ¹H NMR (400 MHz, DMSO) δ=7.96 (s, 1H), 7.42 (brs, 1H), 7.18-7.15 (d, J=12 Hz, 2H), 7.03-6.98 (m, 3H), 6.65 (s, 1H), 6.64-6.58 (d, J=24 Hz, 1H), 6.57-6.51 (d, J=24 Hz, 1H), 4.96 (s, 2H). LCMS: Rt=0.66 min, m/z=404.2 (M+H).

### Example 58: 5-(4-(cyclohexylmethoxy)phenyl)-2-oxo-6-(trifluoromethyl)-1,2-dihydropyridine-3-carboxamide (58)

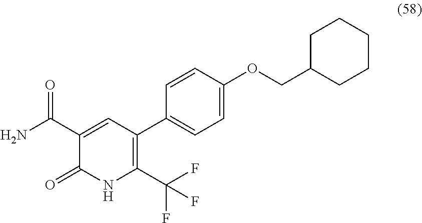 [SMILES: NC(=O)c1cc(-c2ccc(OCC3CCCCC3)cc2)c(C(F)(F)F)[nH]c1=O]

5-(4-(cyclohexylmethoxy)phenyl)-2-oxo-6-(trifluoromethyl)-1,2-dihydropyridine-3-carboxamide (58) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with cyclohexylmethanol. ¹H NMR (400 MHz, DMSO) δ=12.70 (s, 1H), 7.66 (s, 1H), 7.42 (s, 1H), 7.31 (s, 1H), 6.40 (d, J=8.0 Hz, 2H), 6.15 (d, J=8.0 Hz, 2H), 2.97 (d, J=4.0 Hz, 2H), 1.02-0.74 (m, 6H), 0.49-0.28 (m, 3H), 0.26-0.12 (m, 2H). LCMS: Rt=0.94 min, m/z=395.1 (M+H).

### Example 59: 5-(4-((4-methoxybenzyl)oxy)phenyl)-2-oxo-6-(trifluoromethyl)-1,2-dihydropyridine-3-carboxamide (59)

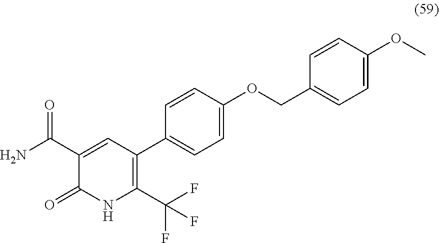 [SMILES: COc1ccc(COc2ccc(-c3cc(C(N)=O)c(=O)[nH]c3C(F)(F)F)cc2)cc1]

5-(4-((4-methoxybenzyl)oxy)phenyl)-2-oxo-6-(trifluoromethyl)-1,2-dihydropyridine-3-carboxamide (59) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 1-(bromomethyl)-4-methoxybenzene. ¹H NMR (400 MHz, DMSO) δ=8.66-8.49 (m, 1H), 8.26 (s, 1H), 8.14 (s, 1H), 7.42 (d, J=8.0 Hz, 2H), 7.27 (d, J=8.0 Hz, 2H), 7.09 (d, J=8.0 Hz, 2H), 6.97 (d, J=8.0 Hz, 2H), 5.07 (s, 2H), 3.77 (s, 3H). LCMS: Rt=0.82 min, m/z=419.3 (M+H).

### Example 60: 5-(4-((4-(dimethylcarbamoyl)benzyl)oxy)phenyl)-2-oxo-6-(trifluoromethyl)-1,2-dihydropyridine-3-carboxamide (60)

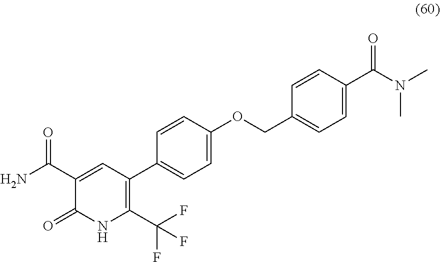 [SMILES: CN(C)C(=O)c1ccc(COc2ccc(-c3cc(C(N)=O)c(=O)[nH]c3C(F)(F)F)cc2)cc1]

5-(4-((4-(dimethylcarbamoyl)benzyl)oxy)phenyl)-2-oxo-6-(trifluoromethyl)-1,2-dihydropyridine-3-carboxamide (60) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)-N,N-dimethylbenzamide. ¹H NMR (400 MHz, DMSO) δ=8.58-8.40 (m, 1H), 8.29 (s, 1H), 8.24-8.16 (m, 1H), 7.54 (d, J=8.0 Hz, 2H), 7.49-7.41 (m, 2H), 7.30 (d, J=8.0 Hz, 2H), 7.12 (d, J=8.0 Hz, 2H), 5.21 (s, 2H), 2.99 (s, 3H), 2.92 (s, 3H). LCMS: Rt=0.76 min, m/z=460.1 (M+H).

### Example 61: 5-(4-((3-methoxybenzyl)oxy)phenyl)-2-oxo-6-(trifluoromethyl)-1,2-dihydropyridine-3-carboxamide (61)

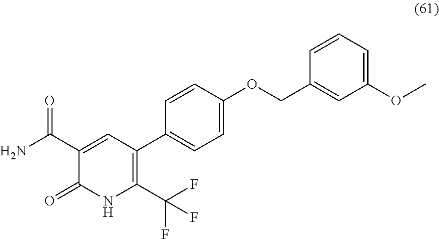 [SMILES: COc1cccc(COc2ccc(-c3cc(C(N)=O)c(=O)[nH]c3C(F)(F)F)cc2)c1]

5-(4-((3-methoxybenzyl)oxy)phenyl)-2-oxo-6-(trifluoromethyl)-1,2-dihydropyridine-3-carboxamide (61) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 1-(bromomethyl)-3-methoxybenzene. ¹H NMR (400 MHz, DMSO) δ=8.52-8.35 (m, 1H), 8.24 (s, 1H), 8.17-8.04 (m, 1H), 7.67-7.48 (m, 1H), 7.40-7.23 (m, 2H), 7.18-7.01 (m, 4H), 6.98-6.85 (m, 1H), 5.13 (s, 2H), 3.77 (s, 3H). LCMS: Rt=0.89 min, m/z=419.3 (M+H).

### Example 62: 5-(4-((3-(methylcarbamoyl)benzyl)oxy)phenyl)-2-oxo-6-(trifluoromethyl)-1,2-dihydropyridine-3-carboxamide (62)

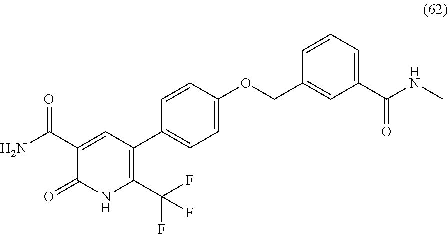 [SMILES: CNC(=O)c1cccc(COc2ccc(-c3cc(C(N)=O)c(=O)[nH]c3C(F)(F)F)cc2)c1]

5-(4-((3-(methylcarbamoyl)benzyl)oxy)phenyl)-2-oxo-6-(trifluoromethyl)-1,2-dihydropyridine-3-carboxamide (62) was made using a procedure similar to that used to make 5-(4-((3-nitrobenzyloxy)phenyl)-2-oxo-6-(trifluoromethyl)-1,2-dihydropyridine-3-carboxamide (54), except 1-(bromomethyl)-3-nitrobenzene was replaced with 3-(bromomethyl)-N-methylbenzamide. 1H NMR (400 MHz, DMSO) δ=8.60-8.45 (m, 1H), 8.29 (s, 1H), 8.23-8.15 (m, 1H), 7.96 (s, 1H), 7.89-7.77 (m, 1H), 7.69-7.59 (m, 1H), 7.50 (s, 1H), 7.30 (d, J=8.0 Hz, 2H), 7.12 (d, J=8.0 Hz, 2H), 5.22 (s, 2H), 2.79 (d, J=4.0 Hz, 3H). LCMS: Rt=0.17 min, m/z=446.1 (M+H).

### Example 63: 2-oxo-5-(4-((tetrahydro-2H-pyran-4-yl)oxy)phenyl)-6-(trifluoromethyl)-1,2-dihydropyridine-3-carboxamide (63)

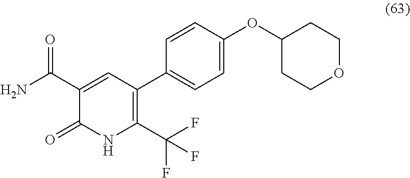 [SMILES: NC(=O)c1cc(-c2ccc(OC3CCOCC3)cc2)c(C(F)(F)F)[nH]c1=O]

2-oxo-5-(4-((tetrahydro-2H-pyran-4-yl)oxy)phenyl)-6-(trifluoromethyl)-1,2-dihydropyridine-3-carboxamide (63) was made using a procedure similar to that used to make 5-(4-((1-acetylpiperidin-4-ylmethoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with tetrahydro-2H-pyran-4-ol. ¹H NMR (400 MHz, DMSO) δ=8.79 (s, 1H), 8.23 (s, 1H), 8.07 (s, 1H), 7.26 (d, J=8.8 Hz, 2H), 7.07 (d, J=8.4 Hz, 2H), 4.65-4.60 (m, 1H), 3.89-3.85 (m, 2H), 3.53-3.47 (m, 2H), 2.01-1.64 (m, 2H), 1.62-1.58 (m, 2H). LCMS: Rt=0.78 min, m/z=383.0 (M+H).

### Example 64: 5-(4-((3-(methylsulfonamido)benzyl)oxy)phenyl)-2-oxo-6-(trifluoromethyl)-1,2-dihydropyridine-3-carboxamide (64)

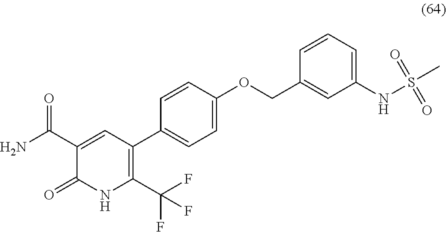 [SMILES: CS(=O)(=O)Nc1cccc(COc2ccc(-c3cc(C(N)=O)c(=O)[nH]c3C(F)(F)F)cc2)c1]

5-(4-((3-(methylsulfonamido)benzyl)oxy)phenyl)-2-oxo-6-(trifluoromethyl)-1,2-dihydropyridine-3-carboxamide (64) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with N-(3-(bromomethyl)phenyl)methanesulfonamide. ¹H NMR (400 MHz, DMSO) δ=9.83 (s, 1H), 8.47 (br, s, 1H), 8.27 (s, 1H), 8.18 (br, s, 1H), 7.36 (m, 1H), 7.31 (m, 1H), 7.21 (m, 2H), 7.20 (m, 2H), 7.10 (m, 2H), 5.14 (s, 2H), 2.99 (s, 3H). LCMS: Rt=0.72 min, m/z=482.0 (M+H).

### Example 65: 5-(4-((3-carbamoylbenzyl)oxy)phenyl)-2-oxo-6-(trifluoromethyl)-1,2-dihydropyridine-3-carboxamide (65)

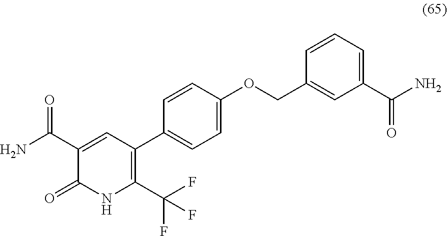 [SMILES: NC(=O)c1cccc(COc2ccc(-c3cc(C(N)=O)c(=O)[nH]c3C(F)(F)F)cc2)c1]

5-(4-((3-carbamoylbenzyl)oxy)phenyl)-2-oxo-6-(trifluoromethyl)-1,2-dihydropyridine-3-carboxamide (65) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)benzamide. ¹H NMR (400 MHz, DMSO) δ=8.00 (s, 3H), 7.85 (d, J=8.0 Hz, 1H), 7.64 (d, J=8.0 Hz, 1H), 7.55-7.45 (m, 1H), 7.41 (s, 1H), 7.20 (d, J=8.0 Hz, 2H), 7.06 (d, J=8.0 Hz, 2H), 5.18 (s, 2H). LCMS: Rt=0.78 min, m/z=432.1 (M+H).

### Example 66: 2-oxo-5-(4-((3-(propylamino)benzyl)oxy)phenyl)-6-(trifluoromethyl)-1,2-dihydropyridine-3-carboxamide (66)

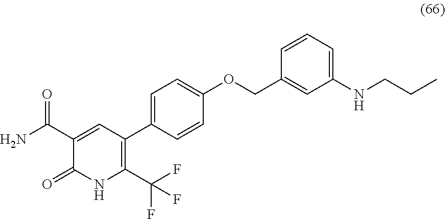 [SMILES: CCCNc1cccc(COc2ccc(-c3cc(C(N)=O)c(=O)[nH]c3C(F)(F)F)cc2)c1]

2-oxo-5-(4-((3-(propylamino)benzyl)oxy)phenyl)-6-(trifluoromethyl)-1,2-dihydropyridine-3-carboxamide (66) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)-N-propylaniline. ¹H NMR (400 MHz, DMSO) δ=8.46 (br s, 1H), 8.28 (s, 1H), 8.20 (br s, 1H), 7.27 (d, J=8.0 Hz, 2H), 7.17 (t, J=7.7 Hz, 1H), 7.08 (d, J=8.0 Hz, 2H), 6.81 (br s, 1H), 6.76-6.66 (m, 2H), 3.02 (t, J=7.2 Hz, 2H), 1.58-1.53 (m, 2H), 0.94 (t, J=7.4 Hz, 3H). LCMS: Rt=0.74 min, m/z=446.3 (M+H).

### Example 67: 5-(4-((3-(dimethylcarbamoyl)benzyl)oxy)phenyl)-2-oxo-6-(trifluoromethyl)-1,2-dihydropyridine-3-carboxamide (67)

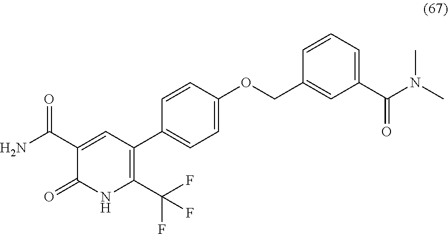 [SMILES: CN(C)C(=O)c1cccc(COc2ccc(-c3cc(C(N)=O)c(=O)[nH]c3C(F)(F)F)cc2)c1]

5-(4-((3-(dimethylcarbamoyl)benzyl)oxy)phenyl)-2-oxo-6-(trifluoromethyl)-1,2-dihydropyridine-3-carboxamide (67) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 3-(bromomethyl)-N,N-dimethylbenzamide. ¹H NMR (400 MHz, DMSO) δ=7.54 (s, 1H), 7.51-7.45 (m, 3H), 7.40-7.34 (m, 1H), 7.17 (d, J=8.0 Hz, 2H), 7.04 (d, J=8.0 Hz, 2H), 5.27-5.14 (m, 2H), 3.04-2.97 (m, 3H), 2.89 (s, 3H). LCMS: Rt=0.84 min, m/z=460.0 (M+H).

### Example 68: 5-(4-((3-acetamidobenzyl)oxy)phenyl)-2-oxo-6-(trifluoromethyl)-1,2-dihydropyridine-3-carboxamide (68)

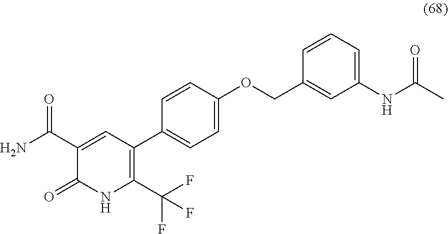 [SMILES: CC(=O)Nc1cccc(COc2ccc(-c3cc(C(N)=O)c(=O)[nH]c3C(F)(F)F)cc2)c1]

5-(4-((3-acetamidobenzyl)oxy)phenyl)-2-oxo-6-(trifluoromethyl)-1,2-dihydropyridine-3-carboxamide (68) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with N-(3-(bromomethyl)phenyl)acetamide. ¹H NMR (400 MHz, DMSO) δ=10.01 (s, 1H), 8.48 (brs, 1H), 8.29 (s, 1H), 8.21 (br s, 1H), 7.71 (s, 1H), 7.55 (m, 1H), 7.35-7.32 (m, 1H), 7.29 (d, J=8.8 Hz, 2H), 7.14 (m, 1H), 7.10 (d, J=8.8 Hz, 2H), 5.13 (s, 2H), 2.05 (s, 3H). LCMS: Rt=0.75 min, m/z=446.3 (M+H).

### Example 69: 5-(4-((4-(methylcarbamoyl)benzyl)oxy)phenyl)-2-oxo-6-(trifluoromethyl)-1,2-dihydropyridine-3-carboxamide (69)

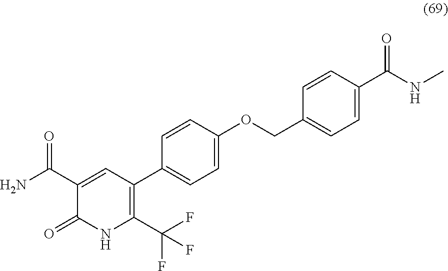 [SMILES: CNC(=O)c1ccc(COc2ccc(-c3cc(C(N)=O)c(=O)[nH]c3C(F)(F)F)cc2)cc1]

5-(4-((4-(methylcarbamoyl)benzyl)oxy)phenyl)-2-oxo-6-(trifluoromethyl)-1,2-dihydropyridine-3-carboxamide (69) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)-N-methylbenzamide. ¹H NMR (400 MHz, DMSO) δ=8.57-8.42 (m, 1H), 8.32-8.24 (m, 1H), 8.21-8.12 (m, 1H), 7.87 (d, J=8.2 Hz, 2H), 7.56 (d, J=8.2 Hz, 2H), 7.29 (d, J=8.6 Hz, 2H), 7.11 (d, J=8.6 Hz, 2H), 5.23 (s, 2H), 2.79 (m, 3H). LCMS: Rt=0.67 min, m/z=446.1 (M+H).

### Example 70: 2-oxo-5-(4-(pyridin-4-ylmethoxy)phenyl)-6-(trifluoromethyl)-1,2-dihydropyridine-3-carboxamide (70)

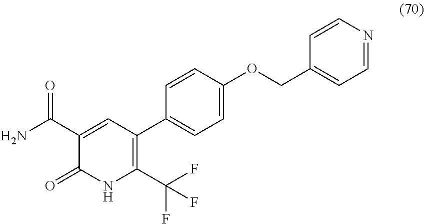 [SMILES: NC(=O)c1cc(-c2ccc(OCc3ccncc3)cc2)c(C(F)(F)F)[nH]c1=O]

2-oxo-5-(4-(pyridin-4-ylmethoxy)phenyl)-6-(trifluoromethyl)-1,2-dihydropyridine-3-carboxamide (70) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)pyridine. ¹H NMR (400 MHz, MeOD) δ=8.57 (d, J=6.0 Hz, 2H), 8.13 (s, 1H), 7.59 (d, J=8.8 Hz, 2H), 7.25 (d, J=8.8 Hz, 2H), 7.07 (d, J=8.8 Hz, 2H), 5.25 (s, 2H). LCMS: Rt=0.77 min, m/z=390.0 (M+H).

### Example 71: 5-(4-(benzo[c][1,2,5]oxadiazol-5-ylmethoxy)phenyl)-2-oxo-6-(trifluoromethyl)-1,2-dihydropyridine-3-carboxamide (71)

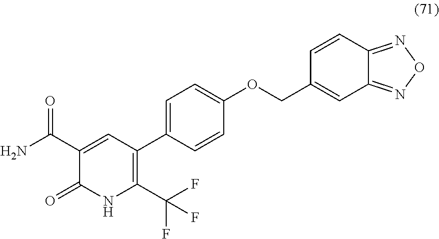 [SMILES: NC(=O)c1cc(-c2ccc(OCc3ccc4nonc4c3)cc2)c(C(F)(F)F)[nH]c1=O]

5-(4-(benzo[c][1,2,5]oxadiazol-5-ylmethoxy)phenyl)-2-oxo-6-(trifluoromethyl)-1,2-dihydropyridine-3-carboxamide (71) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(bromomethyl)benzo[c][1,2,5]oxadiazole. ¹H NMR (400 MHz, DMSO) δ=13.93-13.31 (m, 1H), 8.48 (s, 1H), 8.29 (s, 1H), 8.18 (s, 1H), 8.15-8.08 (m, 2H), 7.69 (d, J=9.5 Hz, 1H), 7.33 (d, J=8.4 Hz, 2H), 7.19 (d, J=8.4 Hz, 2H), 5.34 (s, 2H). LCMS: Rt=0.83 min, m/z=431.0 (M+H).

### Example 72: 2-oxo-5-(4-(pyridin-3-ylmethoxy)phenyl)-6-(trifluoromethyl)-1,2-dihydropyridine-3-carboxamide (72)

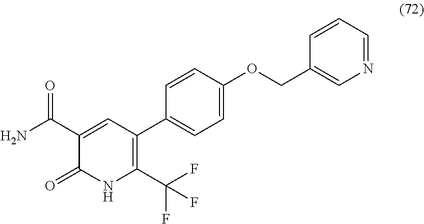 [SMILES: NC(=O)c1cc(-c2ccc(OCc3cccnc3)cc2)c(C(F)(F)F)[nH]c1=O]

2-oxo-5-(4-(pyridin-3-ylmethoxy)phenyl)-6-(trifluoromethyl)-1,2-dihydropyridine-3-carboxamide (72) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)pyridine. ¹H NMR (400 MHz, MeOD) δ=8.71 (s, 1H), 8.57 (d, J=4.0 Hz, 1H), 8.08 (s., 1H), 7.92 (d, J=8.0 Hz, 1H), 7.51-7.39 (m, 1H), 7.24 (d, J=8.0 Hz, 2H), 7.10 (s., 2H), 5.20 (s, 2H). LCMS: Rt=0.77 min, m/z=390.0 (M+H).

### Example 73: 2-oxo-5-(4-(pyridin-2-ylmethoxy)phenyl)-6-(trifluoromethyl)-1,2-dihydropyridine-3-carboxamide (73)

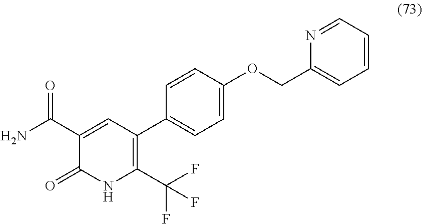 [SMILES: NC(=O)c1cc(-c2ccc(OCc3ccccn3)cc2)c(C(F)(F)F)[nH]c1=O]

2-oxo-5-(4-(pyridin-2-ylmethoxy)phenyl)-6-(trifluoromethyl)-1,2-dihydropyridine-3-carboxamide (73) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)pyridine. ¹H NMR (400 MHz, DMSO) δ=8.65-8.55 (m, 1H), 8.04 (s, 1H), 7.90-7.82 (m, 1H), 7.56 (d, J=8.0 Hz, 1H), 7.37 (m, 1H), 7.22 (d, J=8.0 Hz, 2H), 7.07 (d, J=8.0 Hz, 2H), 5.22 (s, 2H). LCMS: Rt=0.84 min, m/z=390.0 (M+H).

### Example 74: 5-(4-(benzo[c][1,2,5]oxadiazol-4-ylmethoxy)phenyl)-2-oxo-6-(trifluoromethyl)-1,2-dihydropyridine-3-carboxamide (74)

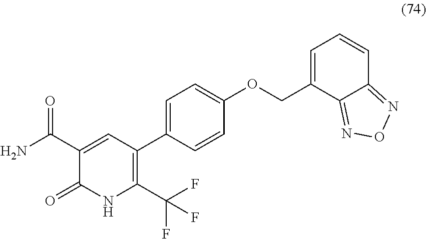 [SMILES: NC(=O)c1cc(-c2ccc(OCc3cccc4nonc34)cc2)c(C(F)(F)F)[nH]c1=O]

5-(4-(benzo[c][1,2,5]oxadiazol-4-ylmethoxy)phenyl)-2-oxo-6-(trifluoromethyl)-1,2-dihydropyridine-3-carboxamide (74) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)benzo[c][1,2,5]oxadiazole. ¹H NMR (400 MHz, DMSO) δ=13.76-13.59 (m, 1H), 8.58-8.43 (m, 1H), 8.30 (s, 1H), 8.19 (s, 1H), 8.06 (d, J=8.0 Hz, 1H), 7.76-7.71 (m, 1H), 7.68 (d, J=8.0 Hz, 1H), 7.32 (d, J=8.0 Hz, 2H), 7.19 (d, J=8.0 Hz, 2H), 5.70-5.48 (m, 2H). LCMS: Rt=0.85 min, m/z=431.1 (M+H).

### Example 75: 5-(4-((3-(methylsulfonyl)benzyl)oxy)phenyl)-2-oxo-6-(trifluoromethyl)-1,2-dihydropyridine-3-carboxamide (75)

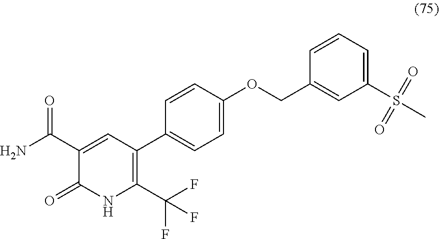 [SMILES: CS(=O)(=O)c1cccc(COc2ccc(-c3cc(C(N)=O)c(=O)[nH]c3C(F)(F)F)cc2)c1]

5-(4-((3-(methylsulfonyl)benzyl)oxy)phenyl)-2-oxo-6-(trifluoromethyl)-1,2-dihydropyridine-3-carboxamide (75) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 1-(bromomethyl)-3-(methylsulfonyl)benzene. ¹H NMR (400 MHz, DMSO) δ=13.66 (s, 1H), 8.48 (s, 1H), 8.30 (s, 1H), 8.20 (s, 1H), 8.06 (s, 1H), 7.94 (s, 1H), 7.85 (s, 1H), 7.74 (s, 1H), 7.32 (d, J=8.0 Hz, 2H), 7.15 (d, J=8.0 Hz, 2H), 5.30 (s, 2H), 3.26 (s, 3H). LCMS: Rt=0.76 min, m/z=467.2 (M+H).

### Example 76: 5-(4-((2-nitrobenzyl)oxy)phenyl)-2-oxo-6-(trifluoromethyl)-1,2-dihydropyridine-3-carboxamide (76)

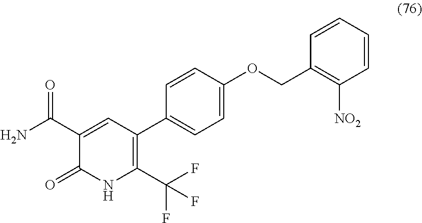 [SMILES: NC(=O)c1cc(-c2ccc(OCc3ccccc3[N+](=O)[O-])cc2)c(C(F)(F)F)[nH]c1=O]

5-(4-((2-nitrobenzyl)oxy)phenyl)-2-oxo-6-(trifluoromethyl)-1,2-dihydropyridine-3-carboxamide (76) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 1-(bromomethyl)-2-nitrobenzene. ¹H NMR (400 MHz, DMSO) δ=10.37 (brs, 1H), 8.15 (d, J=8.2 Hz, 1H), 7.90 (br. s., 1H), 7.85-7.82 (m, 2H), 7.66-7.62 (m, 1H), 7.18 (d, J=8.0 Hz, 2H), 7.03 (d, J=8.3 Hz, 2H), 5.49 (s, 2H). LCMS: Rt=0.85 min, m/z=434.2 (M+H).

### Example 77: 5-(4-((4-cyanobenzyl)oxy)phenyl)-2-oxo-6-(trifluoromethyl)-1,2-dihydropyridine-3-carboxamide (77)

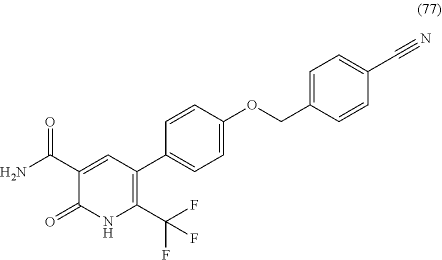 [SMILES: N#Cc1ccc(COc2ccc(-c3cc(C(N)=O)c(=O)[nH]c3C(F)(F)F)cc2)cc1]

5-(4-((4-cyanobenzyl)oxy)phenyl)-2-oxo-6-(trifluoromethyl)-1,2-dihydropyridine-3-carboxamide (77) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 4-(bromomethyl)benzonitrile. ¹H NMR (400 MHz, DMSO) δ=13.65 (br, s, 1H), 8.49 (s., 1H), 8.27 (s, 1H), 8.19 (s, 1H), 7.91 (d, J=8.0 Hz, 2H), 7.69 (d, J=8.0 Hz, 2H), 7.31 (d, J=8.0 Hz, 2H), 7.12 (d, J=8.0 Hz, 2H), 5.29 (s, 2H). LCMS: Rt=1.02 min, m/z=414.0 (M+H).

### Example 78: 5-(4-((3-cyanobenzyl)oxy)phenyl)-2-oxo-6-(trifluoromethyl)-1,2-dihydropyridine-3-carboxamide (78)

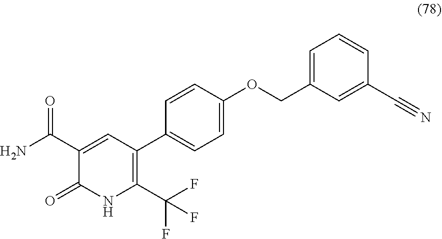 [SMILES: N#Cc1cccc(COc2ccc(-c3cc(C(N)=O)c(=O)[nH]c3C(F)(F)F)cc2)c1]

5-(4-((3-cyanobenzyl)oxy)phenyl)-2-oxo-6-(trifluoromethyl)-1,2-dihydropyridine-3-carboxamide (78) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)benzonitrile. ¹H NMR (400 MHz, DMSO) δ=13.78-13.42 (m, 1H), 8.49 (s., 1H), 8.29 (s, 1H), 8.20 (s., 1H), 7.96 (s, 1H), 7.84 (d, J=8.0 Hz, 2H), 7.71-7.54 (m, 1H), 7.30 (d, J=8.0 Hz, 2H), 7.13 (d, J=8.0 Hz, 2H), 5.23 (s, 2H). LCMS: Rt=0.82 min, m/z=414.1 (M+H).

### Example 79: 2-oxo-6-(trifluoromethyl)-5-(4-((3-(trifluoromethyl)benzyl)oxy)phenyl)-1,2-dihydropyridine-3-carboxamide (79)

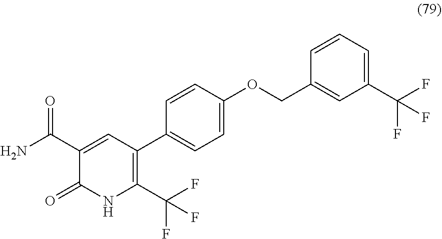 [SMILES: NC(=O)c1cc(-c2ccc(OCc3cccc(C(F)(F)F)c3)cc2)c(C(F)(F)F)[nH]c1=O]

2-oxo-6-(trifluoromethyl)-5-(4-((3-(trifluoromethylbenzyl)oxy)phenyl)-1,2-dihydropyridine-3-carboxamide (79) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 1-(bromomethyl)-3-(trifluoromethyl)benzene. ¹H NMR (400 MHz, DMSO) δ=13.66 (br, s, 1H), 8.48 (s, 1H), 8.28 (s, 1H), 8.21 (s, 1H), 7.85 (s, 1H), 7.81 (d, J=8.0 Hz, 1H), 7.72 (m, 1H), 7.68 (d, J=8.0 Hz, 1H), 7.31 (d, J=8.0 Hz, 2H), 7.13 (d, J=8.0 Hz, 2H), 5.28 (s, 2H). LCMS: Rt=0.91 min, m/z=457.1 (M+H).

### Example 80: (R or S)-2-oxo-5-(4-(1-(pyridin-3-yl)ethoxy)phenyl)-6-(trifluoromethyl)-1,2-dihydropyridine-3-carboxamide; (SFC Peak 1, Chiral) (80)

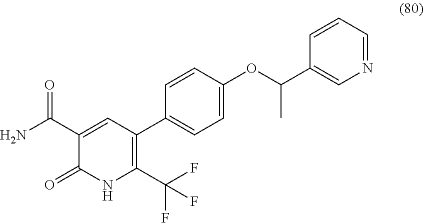 [SMILES: CC(Oc1ccc(-c2cc(C(N)=O)c(=O)[nH]c2C(F)(F)F)cc1)c1cccnc1]

(R or S)-2-oxo-5-(4-(1-(pyridin-3-yl)ethoxy)phenyl)-6-(trifluoromethyl)-1,2-dihydropyridine-3-carboxamide; (SFC peak 1, Chiral) (80) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 3-(1-bromoethyl)pyridine. Racemic mixture was separated using chiral SFC following elution on a Chiralcel-OD-H 20×250 mm semi-prep column, with a mobile phase of methanol (5-40%) in CO₂+DEA (0.05%) and a flow rate of 60-100 mL/min (“Chiral HPLC” conditions herein). ¹H NMR (400 MHz, DMSO) δ=14.09-13.22 (m, 1H), 8.92-8.63 (m, 2H), 8.50 (d, J=4.8 Hz, 1H), 8.19 (s, 1H), 8.04 (s, 1H), 7.85 (d, J=7.9 Hz, 1H), 7.40 (d J=7.8 Hz, 1H), 7.20 (d, J=8.5 Hz, 2H), 7.01 (d, J=8.7 Hz, 2H), 5.68 (m, 1H), 1.61 (d, J=6.4 Hz, 3H). LCMS: Rt=0.89 min, m/z=404.1 (M+H). SFC: Rt=2.33 min, e.e. 92%.

### Example 81: (R or S)-2-oxo-5-(4-(1-(pyridin-3-yl)ethoxy)phenyl)-6-(trifluoromethyl)-1,2-dihydropyridine-3-carboxamide; (SFC Peak 2, Chiral) (81)

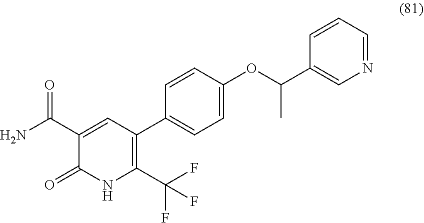 [SMILES: CC(Oc1ccc(-c2cc(C(N)=O)c(=O)[nH]c2C(F)(F)F)cc1)c1cccnc1]

(R or S)-2-oxo-5-(4-(1-(pyridin-3-yl)ethoxy)phenyl)-6-(trifluoromethyl)-1,2-dihydropyridine-3-carboxamide; (SFC peak 1, Chiral) (81) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 3-(1-bromoethyl)pyridine. ¹H NMR (400 MHz, DMSO) δ=9.08-8.79 (m, 1H), 8.68 (d, J=1.9 Hz, 1H), 8.50 (d, J=4.8 Hz, 1H), 8.15 (s, 1H), 7.95 (s, 1H), 7.85 (d, J=7.9 Hz, 1H), 7.40 (d J=8.1 Hz, 1H), 7.18 (d, J=8.7 Hz, 2H), 7.00 (d, J=8.8 Hz, 2H), 5.67 (m, 1H), 1.61 (d, J=6.4 Hz, 3H). LCMS: Rt=0.89 min, m/z=404.1 (M+H). SFC: Rt=2.48 min, e.e. 82%.

### Example 82: 2-oxo-5-(4-(pyrimidin-5-ylmethoxy)phenyl)-6-(trifluoromethyl)-1,2-dihydropyridine-3-carboxamide (82)

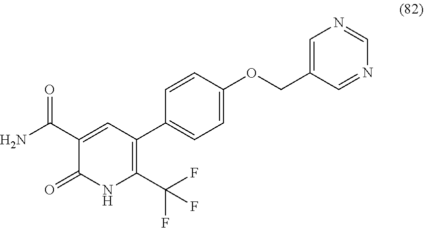 [SMILES: NC(=O)c1cc(-c2ccc(OCc3cncnc3)cc2)c(C(F)(F)F)[nH]c1=O]

2-oxo-5-(4-(pyrimidin-5-ylmethoxy)phenyl)-6-(trifluoromethyl)-1,2-dihydropyridine-3-carboxamide (82) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 5-(bromomethyl)pyrimidine. ¹H NMR (400 MHz, DMSO) δ=10.84-10.38 (m, 1H), 9.20 (s, 1H), 8.96 (s, 2H), 7.84 (s, 1H), 7.23-7.10 (m, 4H), 7.05 (d, J=8.7 Hz, 2H), 5.21 (s, 2H). LCMS: Rt=0.57 min, m/z=413.1 (M+H).

### Example 83: 5-(4-((1-methylpiperidin-4-yl)methoxy)phenyl)-2-oxo-6-(trifluoromethyl)-1,2-dihydropyridine-3-carboxamide (83)

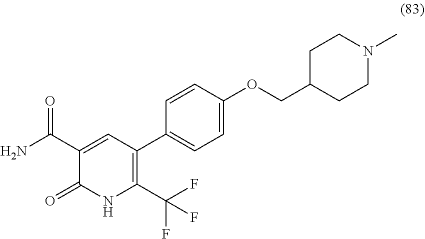 [SMILES: CN1CCC(COc2ccc(-c3cc(C(N)=O)c(=O)[nH]c3C(F)(F)F)cc2)CC1]

5-(4-((1-methylpiperidin-4-yl)methoxy)phenyl)-2-oxo-6-(trifluoromethyl)-1,2-dihydropyridine-3-carboxamide (83) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (1-methylpiperidin-4-yl)methanol. ¹H NMR (400 MHz, DMSO) δ=10.60 (d, J=5.1 Hz, 1H), 7.83 (s, 1H), 7.16-7.07 (m, 3H), 6.91 (d, J=8.7 Hz, 2H), 3.85 (d, J=5.8 Hz, 2H), 3.02-2.91 (m, 2H), 2.32 (s, 3H), 2.17 (s, 2H), 1.89-1.72 (m, 3H), 1.40 (d, J=10.8 Hz, 2H). LCMS: Rt=0.87 min, m/z=410.2 (M+H).

### Example 84: 2-oxo-5-(4-(pyridazin-4-ylmethoxy)phenyl)-6-(trifluoromethyl)-1,2-dihydropyridine-3-carboxamide (84)

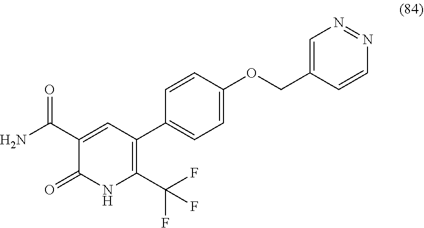 [SMILES: NC(=O)c1cc(-c2ccc(OCc3ccnnc3)cc2)c(C(F)(F)F)[nH]c1=O]

2-oxo-5-(4-(pyridazin-4-ylmethoxy)phenyl)-6-(trifluoromethyl)-1,2-dihydropyridine-3-carboxamide (84) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 4-(bromomethyl)pyridazine. ¹H NMR (400 MHz, DMSO) δ=9.34 (s, 1H), 9.26 (d, J=4.0 Hz, 1H), 8.20 (s, 1H), 8.08 (br. s., 1H), 7.77 (s, 1H), 7.71 (br. s., 1H), 7.26 (d, J=8.0 Hz, 2H), 7.11 (d, J=8.0 Hz, 2H), 5.30 (s, 2H). LCMS: Rt=0.65 min, m/z=391.2 (M+H).

### Example 85: 5-(4-((1H-indazol-6-yl)methoxy)phenyl)-2-oxo-6-(trifluoromethyl)-1,2-dihydropyridine-3-carboxamide (85)

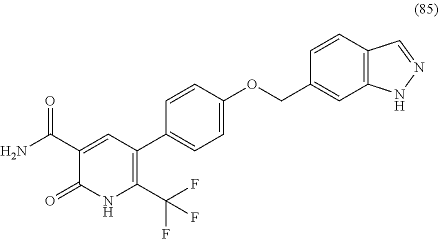 [SMILES: NC(=O)c1cc(-c2ccc(OCc3ccc4cn[nH]c4c3)cc2)c(C(F)(F)F)[nH]c1=O]

5-(4-((1H-indazol-6-yl)methoxy)phenyl)-2-oxo-6-(trifluoromethyl)-1,2-dihydropyridine-3-carboxamide (85) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 6-(bromomethyl)-1H-indazole. ¹H NMR (400 MHz, DMSO) δ=8.50 (br. s., 1H), 8.28 (s, 1H), 8.08 (s, 1H), 7.79 (d, J=8.0 Hz, 1H), 7.65 (s, 1H), 7.29 (d, J=8.0 Hz, 2H), 7.22 (d, J=8.0 Hz, 1H), 7.14 (d, J=8.0 Hz, 2H), 5.31 (s, 2H). LCMS: Rt=0.76 min, m/z=429.2 (M+H).

### Example 86: 5-(4-((1H-indol-6-yl)methoxy)phenyl)-2-oxo-6-(trifluoromethyl)-1,2-dihydropyridine-3-carboxamide (86)

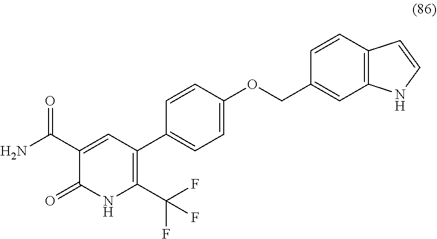 [SMILES: NC(=O)c1cc(-c2ccc(OCc3ccc4cc[nH]c4c3)cc2)c(C(F)(F)F)[nH]c1=O]

5-(4-((1H-indol-6-yl)methoxy)phenyl)-2-oxo-6-(trifluoromethyl)-1,2-dihydropyridine-3-carboxamide (86) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 6-(bromomethyl)-1H-indole. ¹H NMR (400 MHz, DMSO) δ=11.14 (s, 1H), 8.49 (br, s, 1H), 8.27 (s, 1H), 8.17 (br, s, 1H), 7.56 (m, 1H), 7.54 (m, 1H), 7.36 (m, 1H), 7.28 (m, 2H), 7.12 (m, 2H), 6.43 (br. s., 1H), 5.24 (s, 2H). LCMS: Rt=0.82 min, m/z=428.3 (M+H).

### Example 87: 2-oxo-6-(trifluoromethyl)-5-(4-((4-(trifluoromethyl)benzyl)oxy)phenyl)-1,2-dihydropyridine-3-carboxamide (87)

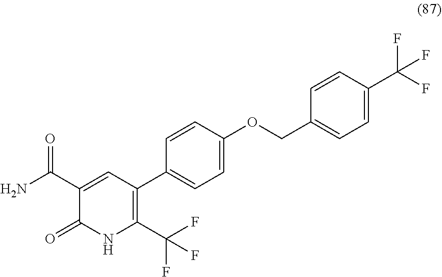 [SMILES: NC(=O)c1cc(-c2ccc(OCc3ccc(C(F)(F)F)cc3)cc2)c(C(F)(F)F)[nH]c1=O]

2-oxo-6-(trifluoromethyl)-5-(4-((4-(trifluoromethyl)benzyl)oxy)phenyl)-1,2-dihydropyridine-3-carboxamide (87) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 1-(bromomethyl)-4-(trifluoromethyl)benzene. ¹H NMR (400 MHz, DMSO) δ=13.65 (br, s, 1H), 8.48 (br, s, 1H), 8.27 (s, 1H), 8.17 (s, 1H), 7.96 (m, 2H), 7.71 (m, 2H), 7.30 (d, J=8.0 Hz, 2H), 7.12 (d, J=8.0 Hz, 2H), 5.28 (s, 2H). LCMS: Rt=0.91 min, m/z=457.1 (M+H).

### Example 88: 2-oxo-5-(4-((5-oxopyrrolidin-3-yl)methoxy)phenyl)-6-(trifluoromethyl)-1,2-dihydropyridine-3-carboxamide (88)

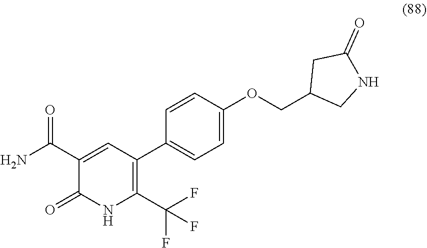 [SMILES: NC(=O)c1cc(-c2ccc(OCC3CNC(=O)C3)cc2)c(C(F)(F)F)[nH]c1=O]

2-oxo-5-(4-((5-oxopyrrolidin-3-yl)methoxy)phenyl)-6-(trifluoromethyl)-1,2-dihydropyridine-3-carboxamide (88) was made using a procedure similar to that used to make 5-(4-((1-acetylpiperidin-4-ylmethoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 4-(hydroxymethyl)pyrrolidin-2-one. ¹H NMR (400 MHz, DMSO) δ=13.80-13.42 (m, 1H), 8.47 (s, 1H), 8.28 (s, 1H), 8.17 (s, 1H), 7.59 (s, 1H), 7.28 (d, J=8.4 Hz, 2H), 7.04 (d, J=8.8 Hz, 2H), 4.06-3.97 (m, 2H), 3.48-3.40 (m, 1H), 3.13 (d, J=9.6 Hz, 1H), 2.86 (s, 1H), 2.38-2.32 (m, 1H), 2.06 (d, J=16.8 Hz, 1H). LCMS: Rt=0.65 min, m/z=379.1 (M+H).

### Example 89: 5-(4-((2-cyanobenzyl)oxy)phenyl)-2-oxo-6-(trifluoromethyl)-1,2-dihydropyridine-3-carboxamide (89)

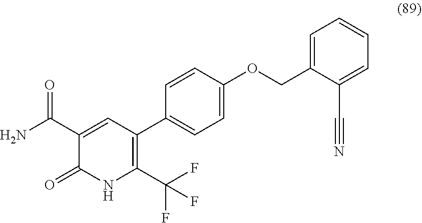 [SMILES: N#Cc1ccccc1COc1ccc(-c2cc(C(N)=O)c(=O)[nH]c2C(F)(F)F)cc1]

5-(4-((2-cyanobenzyl)oxy)phenyl)-2-oxo-6-(trifluoromethyl)-1,2-dihydropyridine-3-carboxamide (89) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)benzonitrile. ¹H NMR (400 MHz, DMSO) δ=13.67 (br, s, 1H), 8.53 (br, s, 1H), 8.28 (s., 1H), 8.20 (m, 1H), 7.95 (d, J=8.0 Hz, 1H), 7.77 (s., 2H), 7.60 (s., 1H), 7.32 (d, J=8.0 Hz, 2H), 7.16 (d, J=8.0 Hz, 2H), 5.31 (s., 2H). LCMS: Rt=0.81 min, m/z=414.0 (M+H).

### Example 90: 2-oxo-5-(4-(tetrazolo[1,5-a]pyridin-7-ylmethoxy)phenyl)-6-(trifluoromethyl)-1,2-dihydropyridine-3-carboxamide (90)

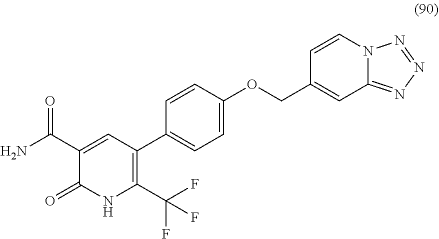 [SMILES: NC(=O)c1cc(-c2ccc(OCc3ccn4nnnc4c3)cc2)c(C(F)(F)F)[nH]c1=O]

2-oxo-5-(4-(tetrazolo[1,5-a]pyridin-7-ylmethoxy)phenyl)-6-(trifluoromethyl)-1,2-dihydropyridine-3-carboxamide (90) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 7-(bromomethyl)tetrazolo[1,5-a]pyridine. ¹H NMR (400 MHz, DMSO) δ=13.86-13.59 (m, 1H), 14.05-13.38 (m, 1H), 9.36 (d, J=7.2 Hz, 1H), 8.48 (s, 1H), 8.29 (s, 2H), 8.19 (s, 1H), 7.53 (d, J=7.0 Hz, 1H), 7.33 (d, J=8.6 Hz, 2H), 7.19 (d, J=8.8 Hz, 2H), 5.42 (s, 2H). LCMS: Rt=0.68 min, m/z=431.0 (M+H).

### Example 91: 5-(4-(imidazo[1,2-a]pyridin-7-ylmethoxy)phenyl)-2-oxo-6-(trifluoromethyl)-1,2-dihydropyridine-3-carboxamide (91)

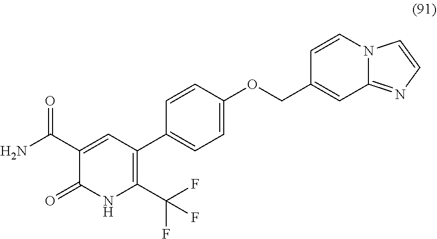 [SMILES: NC(=O)c1cc(-c2ccc(OCc3ccn4ccnc4c3)cc2)c(C(F)(F)F)[nH]c1=O]

5-(4-(imidazo[1,2-a]pyridin-7-ylmethoxy)phenyl)-2-oxo-6-(trifluoromethyl)-1,2-dihydropyridine-3-carboxamide (91) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 7-(bromomethyl)imidazo[1,2-a]pyridine. ¹H NMR (400 MHz, DMSO) δ=8.57 (d, J=4.0 Hz, 1H), 8.29 (br. s., 2H), 7.96 (s, 1H), 7.89 (br. s., 1H), 7.66 (br. s., 1H), 7.58 (s, 1H), 7.18 (d, J=8.0 Hz, 2H), 7.06 (d, J=8.0 Hz, 2H), 7.00-6.98 (m, 1H), 5.19 (s, 2H). LCMS: Rt=0.61 min, m/z=429.3 (M+H).

### Example 92: 5-(4-((1-methylpiperidin-3-yl)methoxy)phenyl)-2-oxo-6-(trifluoromethyl)-1,2-dihydropyridine-3-carboxamide (92)

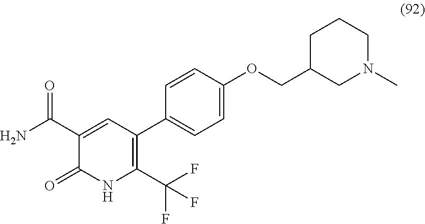 [SMILES: CN1CCCC(COc2ccc(-c3cc(C(N)=O)c(=O)[nH]c3C(F)(F)F)cc2)C1]

5-(4-((1-methylpiperidin-3-yl)methoxy)phenyl)-2-oxo-6-(trifluoromethyl)-1,2-dihydropyridine-3-carboxamide (92) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 3-(bromomethyl)-1-methylpiperidine. ¹H NMR (400 MHz, DMSO) δ=10.77 (s, 1H), 7.81 (s, 1H), 7.11 (d, J=7.2 Hz, 2H), 7.04 (s, 1H), 6.91 (d, J=7.2 Hz, 2H), 3.85 (d, J=8.0 Hz, 2H), 2.84 (d, J=8.2 Hz, 1H), 2.64 (s, 1H), 2.18 (s, 3H), 2.06-1.87 (m, 2H), 1.85-1.70 (m, 2H), 1.64 (s, 1H), 1.51 (d, J=10.2 Hz, 1H), 1.08 (d, J=8.6 Hz, 1H). LCMS: Rt=0.86 min, m/z=410.0 (M+H).

### Example 93: 5-(4-((4-fluoro-3-nitrobenzyl)oxy)phenyl)-2-oxo-6-(trifluoromethyl)-1,2-dihydropyridine-3-carboxamide (93)

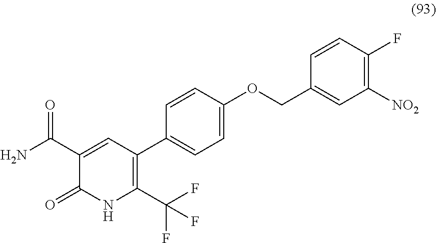 [SMILES: NC(=O)c1cc(-c2ccc(OCc3ccc(F)c([N+](=O)[O-])c3)cc2)c(C(F)(F)F)[nH]c1=O]

5-(4-((4-fluoro-3-nitrobenzyloxy)phenyl)-2-oxo-6-(trifluoromethyl)-1,2-dihydropyridine-3-carboxamide (93) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 4-(bromomethyl)-1-fluoro-2-nitrobenzene. ¹H NMR (400 MHz, DMSO) δ=13.80-13.54 (m, 1H), 8.47 (s, 1H), 8.33-8.27 (m, 2H), 8.20 (s, 1H), 7.95 (d, J=8.6 Hz, 1H), 7.67 (d, J=11.4 Hz, 1H), 7.31 (d, J=8.6 Hz, 2H), 7.14 (d, J=8.8 Hz, 2H), 5.27 (s, 2H). LCMS: Rt=0.91 min, m/z=452.0 (M+H).

### Example 94: 6-((4-(5-carbamoyl-6-oxo-2-(trifluoromethyl)-1,6-dihydropyridin-3-yl)phenoxy)methylbenzo[c][1,2,5]oxadiazole 1-oxide (94)

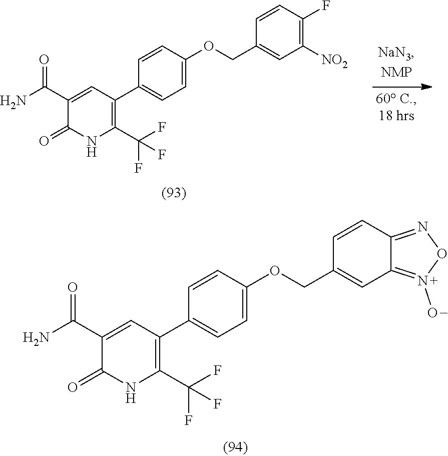

To a solution of 5-(4-((4-fluoro-3-nitrobenzyl)oxy)phenyl)-2-oxo-6-(trifluoromethyl)-1,2-dihydropyridine-3-carboxamide (93) (100 mg, 0.22 mmol, was added sodium azide (43 mg, 0.66 mmol) in NMP (2.2 mL) and the mixture was stirred at 60° C. for 18 hrs. The reaction mixture was poured into water (10 mL), extracted with EA (3 mL×5), dried over Na₂SO₄, filtered, and concentrated. The crude product was purified by Prep-HPLC under acidic conditions (MeCN/water with TFA) to give 6-((4-(5-carbamoyl-6-oxo-2-(trifluoromethyl)-1,6-dihydropyridin-3-yl)phenoxy)methylbenzo[c][1,2,5]oxadiazole 1-oxide. ¹H NMR (400 MHz, DMSO) δ=13.70 (br s, 1H), 8.47 (s, 1H), 8.29 (s, 1H), 8.20 (s, 1H), 7.95-7.45 (m, 3H), 7.32 (d, J=8.4 Hz, 2H), 7.16 (d, J=8.4 Hz, 2H), 5.24 (s, 2H). LCMS: Rt=0.80 min, m/z=447.1 (M+H).

### Example 95: (R or S)-2-oxo-5-(4-(1-(pyrimidin-5-yl)ethoxy)phenyl)-6-(trifluoromethyl)-1,2-dihydropyridine-3-carboxamide; (SFC Peak 1, Chiral) (95)

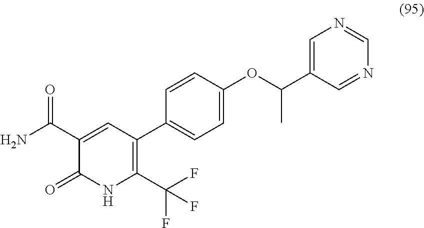 [SMILES: CC(Oc1ccc(-c2cc(C(N)=O)c(=O)[nH]c2C(F)(F)F)cc1)c1cncnc1]

(R or S)-2-oxo-5-(4-(1-(pyrimidin-5-yl)ethoxy)phenyl)-6-(trifluoromethyl)-1,2-dihydropyridine-3-carboxamide; (SFC peak 1, Chiral) (95) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(1-bromoethyl)pyrimidine. ¹H NMR (400 MHz, DMSO) δ=10.93-10.62 (m, 1H), 9.13 (s, 1H), 8.91 (s, 2H), 7.84-7.70 (m, 1H), 7.09 (d, J=8.0 Hz, 2H), 6.96 (d, J=8.2 Hz, 2H), 5.74-5.63 (m, 1H), 1.64 (m, 3H). LCMS: Rt=0.66 min, m/z=405.1 (M+H). SFC: Rt=2.21 min, e.e. 91%.

### Example 96: (R or S)-2-oxo-5-(4-(1-(pyrimidin-5-yl)ethoxy)phenyl)-6-(trifluoromethyl)-1,2-dihydropyridine-3-carboxamide; (SFC Peak 2, Chiral) (96)

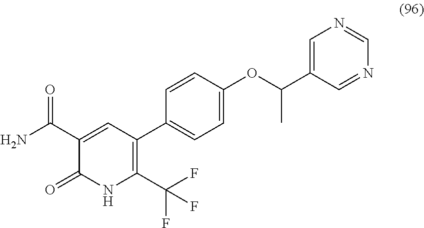 [SMILES: CC(Oc1ccc(-c2cc(C(N)=O)c(=O)[nH]c2C(F)(F)F)cc1)c1cncnc1]

(R or S)-2-oxo-5-(4-(1-(pyrimidin-5-yl)ethoxy)phenyl)-6-(trifluoromethyl)-1,2-dihydropyridine-3-carboxamide; (SFC peak 2, Chiral) (96) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(1-bromoethyl)pyrimidine. ¹H NMR (400 MHz, DMSO) δ=9.19 (s, 1H), 8.97 (s, 2H), 8.58-8.50 (m, 1H), 8.32 (s, 1H), 8.28-8.22 (m, 1H), 7.47-7.36 (m, 1H), 7.30 (d, J=8.8 Hz, 2H), 7.14-7.11 (m, 2H), 5.84-5.77 (m, 1H), 1.72 (m, 3H). LCMS: Rt=0.66 min, m/z=405.1 (M+H). SFC: Rt=2.33 min, e.e. 89%.

### Example 97: (R or S)-5-(4-(1-(3-cyanophenyl)ethoxy)phenyl)-2-oxo-6-(trifluoromethyl)-1,2-dihydropyridine-3-carboxamide; (SFC Peak 1, Chiral) (97)

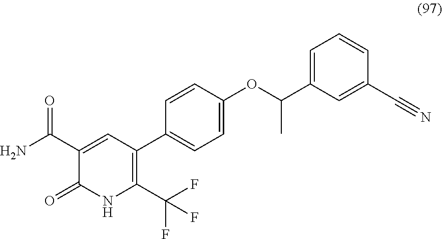 [SMILES: CC(Oc1ccc(-c2cc(C(N)=O)c(=O)[nH]c2C(F)(F)F)cc1)c1cccc(C#N)c1]

(R or S)-5-(4-(1-(3-cyanophenyl)ethoxy)phenyl)-2-oxo-6-(trifluoromethyl)-1,2-dihydropyridine-3-carboxamide; (SFC peak 1, Chiral) (97) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 3-(1-bromoethyl)benzonitrile. ¹H NMR (400 MHz, DMSO) δ=13.81-13.59 (m, 1H), 8.90-8.55 (m, 1H), 8.20 (s, 1H), 8.12-8.05 (m, 1H), 7.93 (s, 1H), 7.83-7.73 (m, 2H), 7.64-7.56 (m, 1H), 7.20 (d, J=8.0 Hz, 2H), 7.00 (d, J=8.0 Hz, 2H), 5.66 (d, J=4.0 Hz, 1H), 1.60 (s, 3H). LCMS: Rt=1.00 min, m/z=428.0 (M+H). SFC: Rt=2.78 min, e.e. 92%.

### Example 98: (R or S)-5-(4-(1-(3-cyanophenyl)ethoxy)phenyl)-2-oxo-6-(trifluoromethyl)-1,2-dihydropyridine-3-carboxamide; (SFC Peak 2, Chiral) (98)

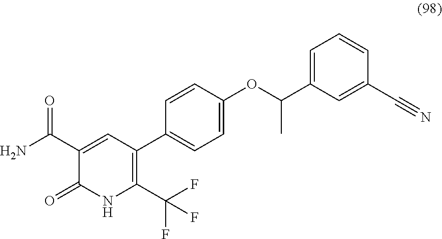 [SMILES: CC(Oc1ccc(-c2cc(C(N)=O)c(=O)[nH]c2C(F)(F)F)cc1)c1cccc(C#N)c1]

(R or S)-5-(4-(1-(3-cyanophenyl)ethoxy)phenyl)-2-oxo-6-(trifluoromethyl)-1,2-dihydropyridine-3-carboxamide; (SFC peak 2, Chiral) (98) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 3-(1-bromoethyl)benzonitrile. ¹H NMR (400 MHz, DMSO) δ=7.98-7.94 (m, 1H), 7.93 (s, 1H), 7.79 (d, J=8.0 Hz, 1H), 7.78-7.74 (m, 1H), 7.62-7.56 (m, 1H), 7.16-7.09 (m, 2H), 6.98-6.91 (m, 2H), 5.79-5.54 (m, 1H), 1.58 (d, J=4.0 Hz, 3H). LCMS: Rt=1.00 min, m/z=428.0 (M+H). SFC: Rt=2.98 min, e.e. 97%.

### Example 99: 5-(4-((5-cyanopyridin-3-yl)methoxy)phenyl)-2-oxo-6-(trifluoromethyl)-1,2-dihydropyridine-3-carboxamide (99)

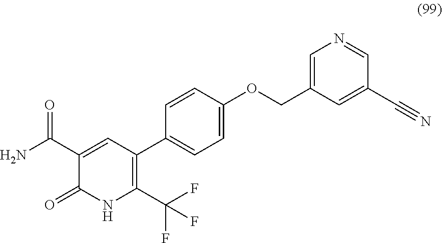 [SMILES: N#Cc1cncc(COc2ccc(-c3cc(C(N)=O)c(=O)[nH]c3C(F)(F)F)cc2)c1]

5-(4-((5-cyanopyridin-3-yl)methoxy)phenyl)-2-oxo-6-(trifluoromethyl)-1,2-dihydropyridine-3-carboxamide (99) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 5-(bromomethyl)nicotinonitrile. ¹H NMR (400 MHz, DMSO) δ=8.95 (d, J=2.0 Hz, 1H), 8.91 (d, J=1.6 Hz, 1H), 8.39 (s, 1H), 8.35 (s, 1H), 8.27 (s, 1H), 7.31 (d, J=8.8 Hz, 2H), 7.14 (d, J=8.4 Hz, 2H), 5.29 (s, 2H). LCMS: Rt=0.74 min, m/z=415.1 (M+H).

### Example 100: (R or S)-5-(4-(1-(benzo[c][1,2,5]oxadiazol-5-yl)ethoxy)phenyl)-2-oxo-6-(trifluoromethyl)-1,2-dihydropyridine-3-carboxamide; (SFC Peak 1, Chiral) (100)

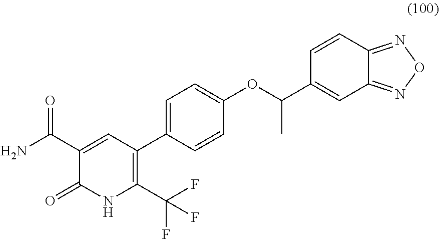 [SMILES: CC(Oc1ccc(-c2cc(C(N)=O)c(=O)[nH]c2C(F)(F)F)cc1)c1ccc2nonc2c1]

(R or S)-5-(4-(1-(benzo[c][1,2,5]oxadiazol-5-yl)ethoxy)phenyl)-2-oxo-6-(trifluoromethyl)-1,2-dihydropyridine-3-carboxamide; (SFC peak 1, Chiral) (100) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(1-bromoethyl)benzo[c][1,2,5]oxadiazole. ¹H NMR (400 MHz, MeOD) δ=8.40 (s, 1H), 8.22 (s, 1H), 8.00-7.88 (m, 2H), 7.66 (d, J=10.2 Hz, 1H), 7.22 (d, J=8.6 Hz, 2H), 7.05 (d, J=8.6 Hz, 2H), 5.65 (d, J=6.0 Hz, 1H), 1.72 (d, J=6.4 Hz, 3H). LCMS: Rt=0.85 min, m/z=445.1 (M+H). SFC: Rt=2.15 min, e.e. 80%.

### Example 101: (R or S)-5-(4-(1-(benzo[c][1,2,5]oxadiazol-5-yl)ethoxy)phenyl)-2-oxo-6-(trifluoromethyl)-1,2-dihydropyridine-3-carboxamide; (SFC Peak 2, Chiral) (101)

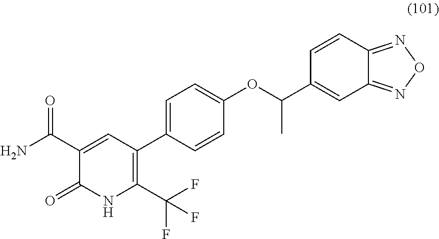 [SMILES: CC(Oc1ccc(-c2cc(C(N)=O)c(=O)[nH]c2C(F)(F)F)cc1)c1ccc2nonc2c1]

(R or S)-5-(4-(1-(benzo[c][1,2,5]oxadiazol-5-yl)ethoxy)phenyl)-2-oxo-6-(trifluoromethyl)-1,2-dihydropyridine-3-carboxamide; (SFC peak 2, Chiral) (101) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(1-bromoethyl)benzo[c][1,2,5]oxadiazole. ¹H NMR (400 MHz, MeOD) δ=8.22 (s, 1H), 7.95 (s, 1H), 7.93-7.92 (m, 2H), 7.67-7.64 (m, 1H), 7.22 (d, J=8.4 Hz, 2H), 7.05 (d, J=8.4 Hz, 2H), 5.65 (d, J=6.0 Hz, 1H), 1.72 (d, J=6.4 Hz, 3H). LCMS: Rt=0.85 min, m/z=445.1 (M+H). SFC: Rt=2.32 min, e.e. 91%.

### Example 102: 2-oxo-5-(4-((tetrahydro-2H-pyran-3-yl)methoxy)phenyl)-6-(trifluoromethyl)-1,2-dihydropyridine-3-carboxamide (102)

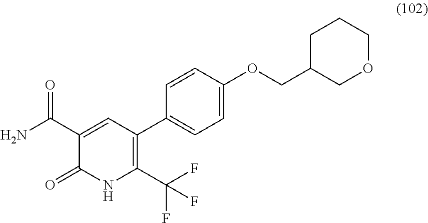 [SMILES: NC(=O)c1cc(-c2ccc(OCC3CCCOC3)cc2)c(C(F)(F)F)[nH]c1=O]

2-oxo-5-(4-((tetrahydro-2H-pyran-3-yl)methoxy)phenyl)-6-(trifluoromethyl)-1,2-dihydropyridine-3-carboxamide (102) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (tetrahydro-2H-pyran-3-yl)methanol. ¹H NMR (500 MHz, DMSO) δ=8.45 (br s, 1H), 8.22 (s, 1H), 8.05 (br s, 1H), 7.25 (d, J=8.5 Hz, 2H), 7.01 (d, J=8.6 Hz, 2H), 3.90 (t, J=5.8 Hz, 3H), 3.76 (d, J=11.2 Hz, 1H), 2.02 (s, 1H), 1.87 (d, J=12.9 Hz, 1H), 1.69-1.46 (m, 2H), 1.42 (dd, J=16.7, 6.6 Hz, 2H). LCMS: Rt=0.72 min, m/z=397.2 (M+H).

### Example 103: 5-(4-([1,2,4]triazolo[4,3-a]pyrimidin-6-ylmethoxy)phenyl)-2-oxo-6-(trifluoromethyl)-1,2-dihydropyridine-3-carboxamide (103)

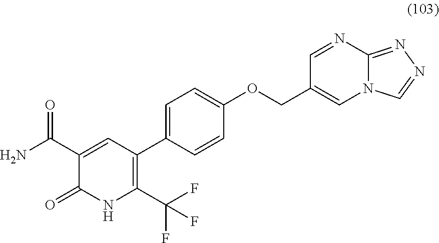 [SMILES: NC(=O)c1cc(-c2ccc(OCc3cnc4nncn4c3)cc2)c(C(F)(F)F)[nH]c1=O]

5-(4-([1,2,4]triazolo[4,3-a]pyrimidin-6-ylmethoxy)phenyl)-2-oxo-6-(trifluoromethyl)-1,2-dihydropyridine-3-carboxamide (103) was made using a procedure similar to that used to make 5-(4-((3-nitrobenzyloxy)phenyl)-2-oxo-6-(trifluoromethyl)-1,2-dihydropyridine-3-carboxamide (54), except 1-(bromomethyl)-3-nitrobenzene was replaced with 6-(bromomethyl)-[1,2,4]triazolo[4,3-a]pyrimidine. ¹H NMR (500 MHz, DMSO) δ=9.61 (s, 1H), 9.08 (s, 1H), 8.72 (s, 1H), 8.33 (s, 2H), 7.86 (s, 1H), 7.30 (s, 1H), 7.19 (d, J=8.0 Hz, 2H), 7.10 (d, J=8.0 Hz, 2H), 5.29 (s, 2H). LCMS: Rt=0.70 min, m/z=431.2 (M+H).

### Example 104: 5-(4-((5-chloro-2-fluoropyridin-3-yl)methoxy)phenyl)-2-oxo-6-(trifluoromethyl)-1,2-dihydropyridine-3-carboxamide (104)

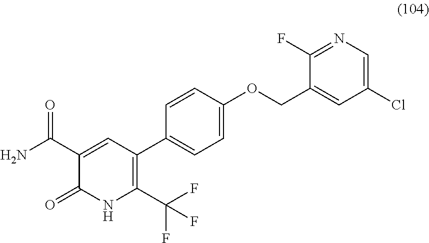 [SMILES: NC(=O)c1cc(-c2ccc(OCc3cc(Cl)cnc3F)cc2)c(C(F)(F)F)[nH]c1=O]

5-(4-((5-chloro-2-fluoropyridin-3-yl)methoxy)phenyl)-2-oxo-6-(trifluoromethyl)-1,2-dihydropyridine-3-carboxamide (104) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 3-(bromomethyl)-5-chloro-2-fluoropyridine. LCMS: Rt=3.23 min, m/z=442.0 (M+H).

### Example 105: 5-(4-((5-chloropyridin-2-yl)methoxy)phenyl)-2-oxo-6-(trifluoromethyl)-1,2-dihydropyridine-3-carboxamide (105)

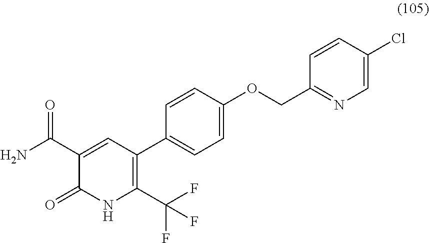 [SMILES: NC(=O)c1cc(-c2ccc(OCc3ccc(Cl)cn3)cc2)c(C(F)(F)F)[nH]c1=O]

5-(4-((5-chloropyridin-2-yl)methoxy)phenyl)-2-oxo-6-(trifluoromethyl)-1,2-dihydropyridine-3-carboxamide (105) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)-5-chloropyridine. LCMS: Rt=3.10 min, m/z=424.0 (M+H).

### Example 106: 5-(4-((2-methoxypyridin-4-yl)methoxy)phenyl)-2-oxo-6-(trifluoromethyl)-1,2-dihydropyridine-3-carboxamide (106)

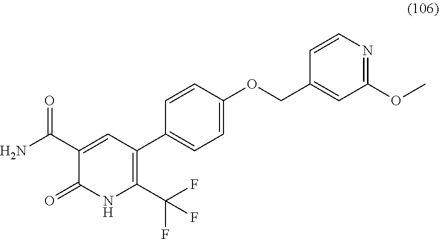 [SMILES: COc1cc(COc2ccc(-c3cc(C(N)=O)c(=O)[nH]c3C(F)(F)F)cc2)ccn1]

5-(4-((2-methoxypyridin-4-yl)methoxy)phenyl)-2-oxo-6-(trifluoromethyl)-1,2-dihydropyridine-3-carboxamide (106) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)-2-methoxypyridine. LCMS: Rt=2.90 min, m/z=420.0 (M+H).

### Example 107: 5-(4-((3,5-dimethylisoxazol-4-yl)methoxy)phenyl)-2-oxo-6-(trifluoromethyl)-1,2-dihydropyridine-3-carboxamide (107)

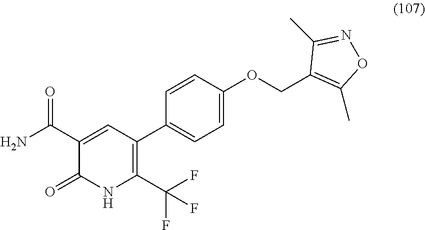 [SMILES: Cc1noc(C)c1COc1ccc(-c2cc(C(N)=O)c(=O)[nH]c2C(F)(F)F)cc1]

5-(4-((3,5-dimethylisoxazol-4-yl)methoxy)phenyl)-2-oxo-6-(trifluoromethyl)-1,2-dihydropyridine-3-carboxamide (107) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)-3,5-dimethylisoxazole. LCMS: Rt=2.90 min, m/z=408.0 (M+H).

### Example 108: 5-(4-((2-chloro-5-nitrobenzyl)oxy)phenyl)-2-oxo-6-(trifluoromethyl)-1,2-dihydropyridine-3-carboxamide (108)

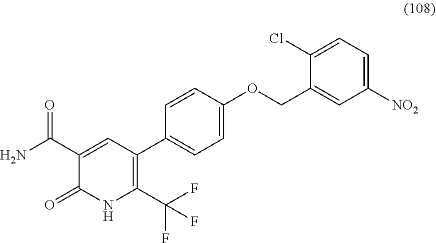 [SMILES: NC(=O)c1cc(-c2ccc(OCc3cc([N+](=O)[O-])ccc3Cl)cc2)c(C(F)(F)F)[nH]c1=O]

5-(4-((2-chloro-5-nitrobenzyl)oxy)phenyl)-2-oxo-6-(trifluoromethyl)-1,2-dihydropyridine-3-carboxamide (108) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)-1-chloro-4-nitrobenzene. LCMS: Rt=3.35 min, m/z=488.0 (M+H).

### Example 109: 5-(4-((1-methyl-3-(trifluoromethyl)-1H-pyrazol-4-yl)methoxy)phenyl)-2-oxo-6-(trifluoromethyl)-1,2-dihydropyridine-3-carboxamide (109)

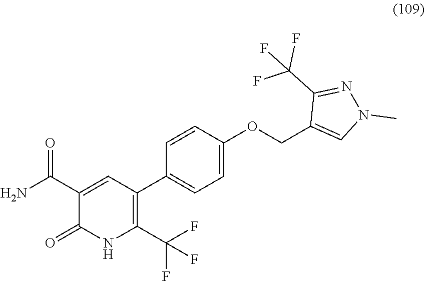 [SMILES: Cn1cc(COc2ccc(-c3cc(C(N)=O)c(=O)[nH]c3C(F)(F)F)cc2)c(C(F)(F)F)n1]

5-(4-((1-methyl-3-(trifluoromethyl)-1H-pyrazol-4-yl)methoxy)phenyl)-2-oxo-6-(trifluoromethyl)-1,2-dihydropyridine-3-carboxamide (109) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)-1-methyl-3-(trifluoromethyl)-1H-pyrazole. LCMS: Rt=3.07 min, m/z=461.1 (M+H).

### Example 110: 2-oxo-5-(4-(thiophen-3-ylmethoxy)phenyl)-6-(trifluoromethyl)-1,2-dihydropyridine-3-carboxamide (110)

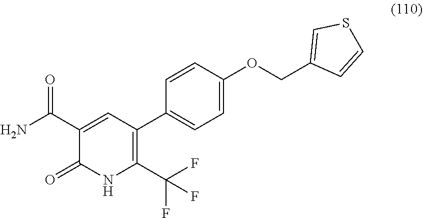 [SMILES: NC(=O)c1cc(-c2ccc(OCc3ccsc3)cc2)c(C(F)(F)F)[nH]c1=O]

2-oxo-5-(4-(thiophen-3-ylmethoxy)phenyl)-6-(trifluoromethyl)-1,2-dihydropyridine-3-carboxamide (110) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)thiophene. LCMS: Rt=3.18 min, m/z=295.0 (M+H).

### Example 111: 2-oxo-6-(trifluoromethyl)-5-(4-((8-(trifluoromethyl)pyridin-3-yl)methoxy)phenyl)-1,2-dihydropyridine-3-carboxamide (111)

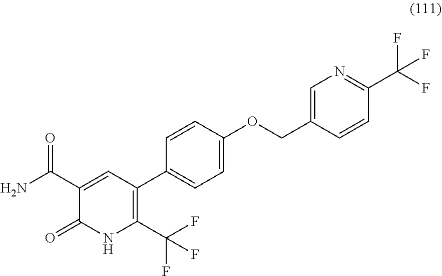 [SMILES: NC(=O)c1cc(-c2ccc(OCc3ccc(C(F)(F)F)nc3)cc2)c(C(F)(F)F)[nH]c1=O]

2-oxo-6-(trifluoromethyl)-5-(4-((8-(trifluoromethyl)pyridin-3-yl)methoxy)phenyl)-1,2-dihydropyridine-3-carboxamide (111) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(bromomethyl)-2-(trifluoromethyl)pyridine. LCMS: Rt=3.21 min, m/z=458.1 (M+H).

### Example 112: 5-(4-((3,4-difluorobenzyl)oxy)phenyl)-2-oxo-6-(trifluoromethyl)-1,2-dihydropyridine-3-carboxamide (112)

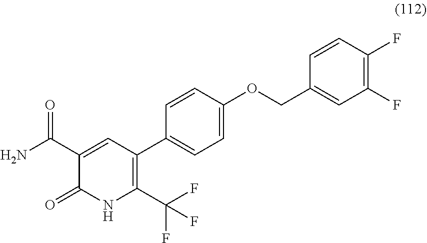 [SMILES: NC(=O)c1cc(-c2ccc(OCc3ccc(F)c(F)c3)cc2)c(C(F)(F)F)[nH]c1=O]

5-(4-((3,4-difluorobenzyl)oxy)phenyl)-2-oxo-6-(trifluoromethyl)-1,2-dihydropyridine-3-carboxamide (112) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 4-(bromomethyl)-1,2-difluorobenzene. LCMS: Rt=3.34 min, m/z=425.0 (M+H).

### Example 113: 5-(4-((4-methoxy-3,5-dimethylpyridin-2-yl)methoxy)phenyl)-2-oxo-6-(trifluoromethyl)-1,2-dihydropyridine-3-carboxamide (113)

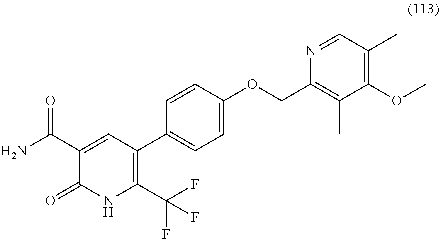 [SMILES: COc1c(C)cnc(COc2ccc(-c3cc(C(N)=O)c(=O)[nH]c3C(F)(F)F)cc2)c1C]

5-(4-((4-methoxy-3,5-dimethylpyridin-2-yl)methoxy)phenyl)-2-oxo-6-(trifluoromethyl)-1,2-dihydropyridine-3-carboxamide (113) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 2-(bromomethyl)-4-methoxy-3,5-dimethylpyridine. LCMS: Rt=2.39 min, m/z=448.0 (M+H).

### Example 114: 5-(4-((3-fluoropyridin-2-yl)methoxy)phenyl)-2-oxo-6-(trifluoromethyl)-1,2-dihydropyridine-3-carboxamide (114)

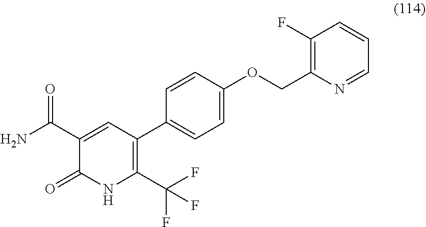 [SMILES: NC(=O)c1cc(-c2ccc(OCc3ncccc3F)cc2)c(C(F)(F)F)[nH]c1=O]

5-(4-((3-fluoropyridin-2-yl)methoxy)phenyl)-2-oxo-6-(trifluoromethyl)-1,2-dihydropyridine-3-carboxamide (114) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)-3-fluoropyridine. LCMS: Rt=2.82 min, m/z=408.0 (M+H).

### Example 115: 2-oxo-5-(4-(thiophen-2-ylmethoxy)phenyl)-6-(trifluoromethyl)-1,2-dihydropyridine-3-carboxamide (115)

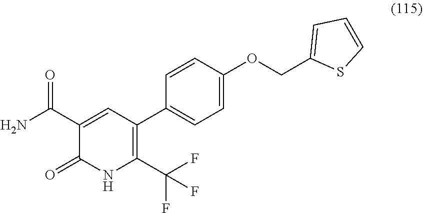 [SMILES: NC(=O)c1cc(-c2ccc(OCc3cccs3)cc2)c(C(F)(F)F)[nH]c1=O]

2-oxo-5-(4-(thiophen-2-ylmethoxy)phenyl)-6-(trifluoromethyl)-1,2-dihydropyridine-3-carboxamide (115) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)thiophene. LCMS: Rt=3.18 min, m/z=395.0 (M+H).

### Example 116: 5-(4-((2,5-difluorobenzyl)oxy)phenyl)-2-oxo-6-(trifluoromethyl)-1,2-dihydropyridine-3-carboxamide (116)

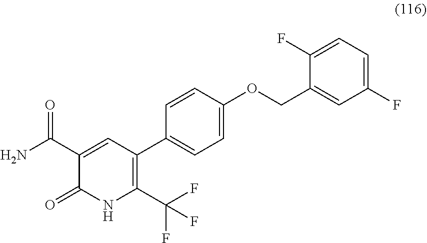 [SMILES: NC(=O)c1cc(-c2ccc(OCc3cc(F)ccc3F)cc2)c(C(F)(F)F)[nH]c1=O]

5-(4-((2,5-difluorobenzyl)oxy)phenyl)-2-oxo-6-(trifluoromethyl)-1,2-dihydropyridine-3-carboxamide (116) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)-1,4-difluorobenzene. LCMS: Rt=3.32 min, m/z=425.0 (M+H).

### Example 117: 5-(4-((4-chloropyridin-2-yl)methoxy)phenyl)-2-oxo-6-(trifluoromethyl)-1,2-dihydropyridine-3-carboxamide (117)

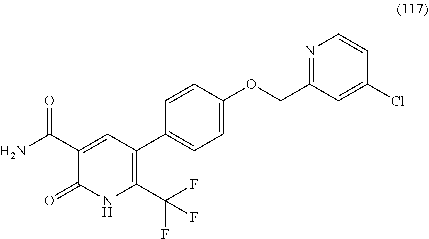 [SMILES: NC(=O)c1cc(-c2ccc(OCc3cc(Cl)ccn3)cc2)c(C(F)(F)F)[nH]c1=O]

5-(4-((4-chloropyridin-2-yl)methoxy)phenyl)-2-oxo-6-(trifluoromethyl)-1,2-dihydropyridine-3-carboxamide (117) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)-4-chloropyridine. LCMS: Rt=3.01 min, m/z=424.0 (M+H).

### Example 118: 5-(4-((2,4-dimethylthiazol-5-yl)methoxy)phenyl)-2-oxo-6-(trifluoromethyl)-1,2-dihydropyridine-3-carboxamide (118)

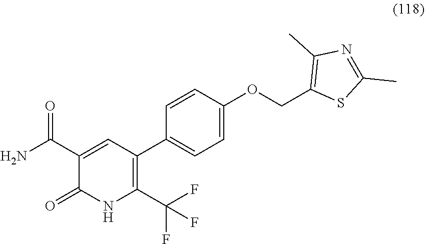 [SMILES: Cc1nc(C)c(COc2ccc(-c3cc(C(N)=O)c(=O)[nH]c3C(F)(F)F)cc2)s1]

5-(4-((2,4-dimethylthiazol-5-yl)methoxy)phenyl)-2-oxo-6-(trifluoromethyl)-1,2-dihydropyridine-3-carboxamide (118) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(bromomethyl)-2,4-dimethylthiazole. LCMS: Rt=2.75 min, m/z=424.1 (M+H).

### Example 119: 5-(4-((2-chloropyridin-3-yl)methoxy)phenyl)-2-oxo-6-(trifluoromethyl)-1,2-dihydropyridine-3-carboxamide (119)

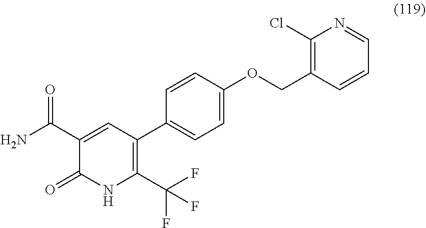 [SMILES: NC(=O)c1cc(-c2ccc(OCc3cccnc3Cl)cc2)c(C(F)(F)F)[nH]c1=O]

5-(4-((2-chloropyridin-3-yl)methoxy)phenyl)-2-oxo-6-(trifluoromethyl)-1,2-dihydropyridine-3-carboxamide (119) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)-2-chloropyridine. LCMS: Rt=3.02 min, m/z=424.0 (M+H).

### Example 120: 2-oxo-5-(4-((5-(propylamino)pyridin-3-yl)methoxy)phenyl)-6-(trifluoromethyl)-1,2-dihydropyridine-3-carboxamide (120)

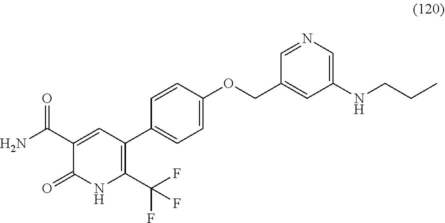 [SMILES: CCCNc1cncc(COc2ccc(-c3cc(C(N)=O)c(=O)[nH]c3C(F)(F)F)cc2)c1]

2-oxo-5-(4-((5-(propylamino)pyridin-3-yl)methoxy)phenyl)-6-(trifluoromethyl)-1,2-dihydropyridine-3-carboxamide (120) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(bromomethyl)-N-propylpyridin-3-amine. ¹H NMR (400 MHz, DMSO) δ=10.72 (s, 1H), 7.92 (d, J=2.4 Hz, 1H), 7.83 (d, J=10.8 Hz, 2H), 7.14 (d, J=8.4 Hz, 2H), 7.05-6.93 (m, 4H), 5.94 (s, 1H), 5.04 (s, 2H), 3.88 (s, 1H), 3.00 (q, J=6.7 Hz, 2H), 1.65-1.49 (m, 2H), 0.94 (m, 3H). LCMS: Rt=0.93 min, m/z=447.0 (M+H).

### Example 121: 2-oxo-5-(4-(pyrimidin-2-ylmethoxy)phenyl)-6-(trifluoromethyl)-1,2-dihydropyridine-3-carboxamide (121)

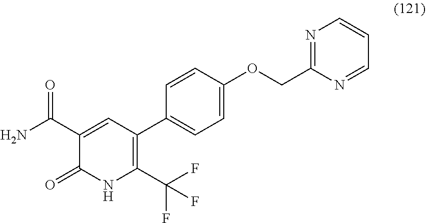 [SMILES: NC(=O)c1cc(-c2ccc(OCc3ncccn3)cc2)c(C(F)(F)F)[nH]c1=O]

2-oxo-5-(4-(pyrimidin-2-ylmethoxy)phenyl)-6-(trifluoromethyl)-1,2-dihydropyridine-3-carboxamide (121) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)pyrimidine. ¹H NMR (400 MHz, DMSO) δ=8.86 (d, J=5.2 Hz, 2H), 8.85 (s, 1H), 8.25 (s, 1H), 8.14 (s, 1H), 7.49 (t, J=4.8 Hz, 1H), 7.25 (d, J=8.4 Hz, 2H), 7.06 (d, J=8.4 Hz, 2H), 5.33 (s, 2H). LCMS: Rt=0.63 min, m/z=391.0 (M+H).

### Example 122: 5-(4-((2-morpholinopyrimidin-5-yl)methoxy)phenyl)-2-oxo-6-(trifluoromethyl)-1,2-dihydropyridine-3-carboxamide (122)

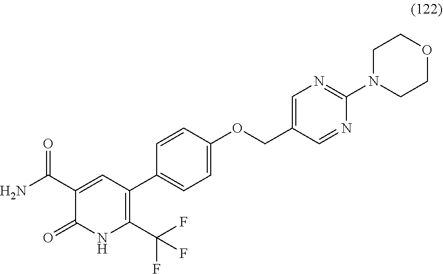 [SMILES: NC(=O)c1cc(-c2ccc(OCc3cnc(N4CCOCC4)nc3)cc2)c(C(F)(F)F)[nH]c1=O]

5-(4-((2-morpholinopyrimidin-5-yl)methoxy)phenyl)-2-oxo-6-(trifluoromethyl)-1,2-dihydropyridine-3-carboxamide (122) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(5-(bromomethyl)pyrimidin-2-yl)morpholine. ¹H NMR (400 MHz, DMSO) δ=9.84 (m, 1H), 8.53 (s, 2H), 8.26 (s, 1H), 7.98 (s, 1H), 7.47 (br s, 1H), 7.19 (d, J=8.4 Hz, 2H), 7.04 (d, J=8.4 Hz, 2H), 4.98 (s, 2H), 3.80 (m, 4H), 3.67 (m, 4H). LCMS: Rt=0.72 min, m/z=476.1 (M+H).

### Example 123: 2-oxo-5-(4-(pyrazin-2-ylmethoxy)phenyl)-6-(trifluoromethyl)-1,2-dihydropyridine-3-carboxamide (123)

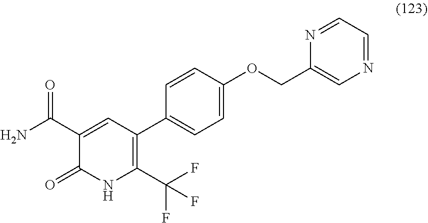 [SMILES: NC(=O)c1cc(-c2ccc(OCc3cnccn3)cc2)c(C(F)(F)F)[nH]c1=O]

2-oxo-5-(4-(pyrazin-2-ylmethoxy)phenyl)-6-(trifluoromethyl)-1,2-dihydropyridine-3-carboxamide (123) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)pyrazine. ¹H NMR (400 MHz, DMSO) δ=10.79 (br s, 1H), 8.86 (m, 1H), 8.82 (s, 1H), 8.72-8.67 (m, 1H), 8.67-8.62 (m, 1H), 7.80 (s, 1H), 7.75 (m, 1H), 7.16 (d, J=8.8 Hz, 2H), 7.06 (d, J=8.8 Hz, 2H), 5.28 (s, 2H). LCMS: Rt=0.65 min, m/z=391.1 (M+H).

### Example 124: (R or S)-5-(4-(1-(5-cyanopyridin-3-yl)ethoxy)phenyl)-2-oxo-6-(trifluoromethyl)-1,2-dihydropyridine-3-carboxamide; (SFC Peak 1, Chiral) (124)

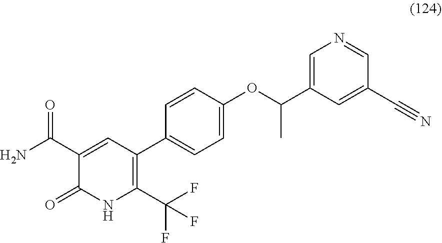 [SMILES: CC(Oc1ccc(-c2cc(C(N)=O)c(=O)[nH]c2C(F)(F)F)cc1)c1cncc(C#N)c1]

(R or S)-5-(4-(1-(5-cyanopyridin-3-yl)ethoxy)phenyl)-2-oxo-6-(trifluoromethyl)-1,2-dihydropyridine-3-carboxamide; (SFC peak 1, Chiral) (124) was made using a procedure similar to that used to make 5-(4-((3-nitrobenzyloxy)phenyl)-2-oxo-6-(trifluoromethyl)-1,2-dihydropyridine-3-carboxamide (54), except 1-(bromomethyl)-3-nitrobenzene was replaced with 5-(1-bromoethyl)nicotinonitrile. ¹H NMR (400 MHz, DMSO) δ=9.33-9.09 (m, 1H), 8.96 (d, J=1.8 Hz, 2H), 8.42 (t, J=2.0 Hz, 1H), 8.10 (s, 1H), 7.82 (s, 1H), 7.19 (d, J=8.0 Hz, 2H), 7.02 (d, J=8.0 Hz, 2H), 5.73 (d, J=8.0 Hz, 1H), 1.63 (d, J=8.0 Hz, 3H), 1.68-1.58 (m, 1H). LCMS: Rt=0.91 min, m/z=429.1 (M+H). SFC: Rt=2.29 min, e.e. 100%.

### Example 125: (R or S)-5-(4-(1-(5-cyanopyridin-3-yl)ethoxy)phenyl)-2-oxo-6-(trifluoromethyl)-1,2-dihydropyridine-3-carboxamide; (SFC Peak 2, Chiral) (125)

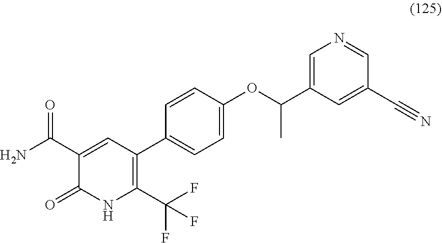 [SMILES: CC(Oc1ccc(-c2cc(C(N)=O)c(=O)[nH]c2C(F)(F)F)cc1)c1cncc(C#N)c1]

(R or S)-5-(4-(1-(5-cyanopyridin-3-yl)ethoxy)phenyl)-2-oxo-6-(trifluoromethyl)-1,2-dihydropyridine-3-carboxamide; (SFC peak 2, Chiral) (125) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(1-bromoethyl)nicotinonitrile. ¹H NMR (400 MHz, DMSO) δ=9.17 (s, 1H), 8.96 (d, J=1.6 Hz, 2H), 8.42 (s, 1H), 8.11 (s, 1H), 7.83 (s, 1H), 7.19 (d, J=8.0 Hz, 2H), 7.02 (d, J=8.0 Hz, 2H), 5.74 (d, J=6.0 Hz, 1H), 1.63 (d, J=6.4 Hz, 3H). LCMS: Rt=0.91 min, m/z=429.1 (M+H). SFC: Rt=3.29 min, e.e. 92%.

### Example 126: 5-(4-((1-methyl-1H-pyrazol-5-yl)methoxy)phenyl)-2-oxo-6-(trifluoromethyl)-1,2-dihydropyridine-3-carboxamide (126)

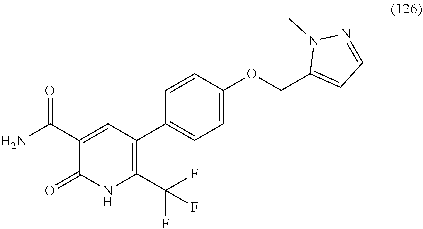 [SMILES: Cn1nccc1COc1ccc(-c2cc(C(N)=O)c(=O)[nH]c2C(F)(F)F)cc1]

5-(4-((1-methyl-1H-pyrazol-5-yl)methoxy)phenyl)-2-oxo-6-(trifluoromethyl)-1,2-dihydropyridine-3-carboxamide (126) was made using a procedure similar to that used to make 5-(4-((3-nitrobenzyloxy)phenyl)-2-oxo-6-(trifluoromethyl)-1,2-dihydropyridine-3-carboxamide (54), except 1-(bromomethyl)-3-nitrobenzene was replaced with 5-(bromomethyl)-1-methyl-1H-pyrazole. ¹H NMR (400 MHz, DMSO) δ=13.64 (s, 1H), 8.46 (s, 1H), 8.28 (s, 1H), 8.17 (s, 1H), 7.39 (d, J=1.6 Hz, 1H), 7.30 (d, J=8.4 Hz, 2H), 7.15 (d, J=8.8 Hz, 2H), 6.40 (d, J=1.6 Hz, 1H), 5.23 (s, 2H), 3.85 (s, 3H). LCMS: Rt=0.66 min, m/z=393.1 (M+H).

### Example 127: 5-(4-((4-methoxypyridin-2-yl)methoxy)phenyl)-2-oxo-6-(trifluoromethyl)-1,2-dihydropyridine-3-carboxamide (127)

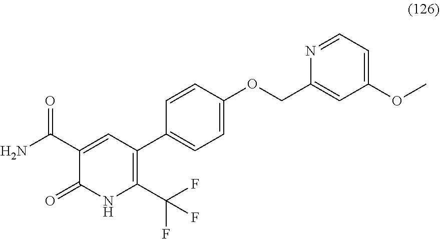 [SMILES: COc1ccnc(COc2ccc(-c3cc(C(N)=O)c(=O)[nH]c3C(F)(F)F)cc2)c1]

5-(4-((4-methoxypyridin-2-yl)methoxy)phenyl)-2-oxo-6-(trifluoromethyl)-1,2-dihydropyridine-3-carboxamide (127) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 2-(bromomethyl)-4-methoxypyridine. LCMS: Rt=2.29 min, m/z=420.1 (M+H).

### Example 128: 5-(4-((8-morpholinopyridin-3-yl)methoxy)phenyl)-2-oxo-6-(trifluoromethyl)-1,2-dihydropyridine-3-carboxamide (128)

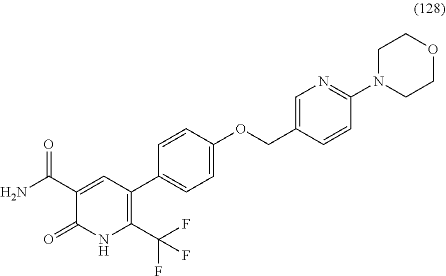 [SMILES: NC(=O)c1cc(-c2ccc(OCc3ccc(N4CCOCC4)nc3)cc2)c(C(F)(F)F)[nH]c1=O]

5-(4-((8-morpholinopyridin-3-yl)methoxy)phenyl)-2-oxo-6-(trifluoromethyl)-1,2-dihydropyridine-3-carboxamide (128) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(5-(bromomethyl)pyridin-2-yl)morpholine. LCMS: Rt=2.34 min, m/z=475.2 (M+H).

### Example 129: 5-(4-((2-fluoropyridin-3-yl)methoxy)phenyl)-2-oxo-6-(trifluoromethyl)-1,2-dihydropyridine-3-carboxamide (129)

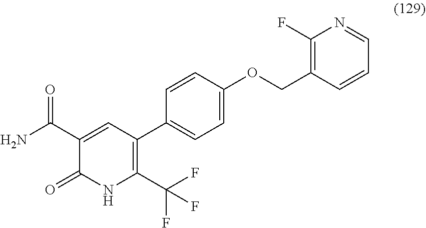 [SMILES: NC(=O)c1cc(-c2ccc(OCc3cccnc3F)cc2)c(C(F)(F)F)[nH]c1=O]

5-(4-((2-fluoropyridin-3-yl)methoxy)phenyl)-2-oxo-6-(trifluoromethyl)-1,2-dihydropyridine-3-carboxamide (129) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)-2-fluoropyridine. LCMS: Rt=2.94 min, m/z=408.1 (M+H).

### Example 130: 5-(4-((5-fluoro-2-methoxypyridin-4-yl)methoxy)phenyl)-2-oxo-6-(trifluoromethyl)-1,2-dihydropyridine-3-carboxamide (130)

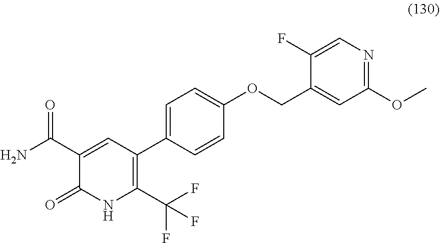 [SMILES: COc1cc(COc2ccc(-c3cc(C(N)=O)c(=O)[nH]c3C(F)(F)F)cc2)c(F)cn1]

5-(4-((5-fluoro-2-methoxypyridin-4-yl)methoxy)phenyl)-2-oxo-6-(trifluoromethyl)-1,2-dihydropyridine-3-carboxamide (130) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)-5-fluoro-2-methoxypyridine. LCMS: Rt=3.16 min, m/z=438.1 (M+H).

### Example 131: 2-oxo-5-(4-(quinoxalin-6-ylmethoxy)phenyl)-6-(trifluoromethyl)-1,2-dihydropyridine-3-carboxamide (131)

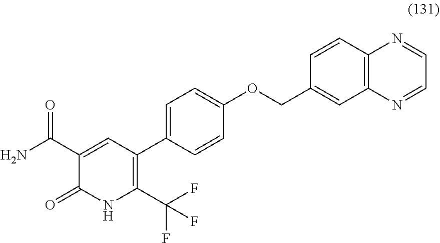 [SMILES: NC(=O)c1cc(-c2ccc(OCc3ccc4nccnc4c3)cc2)c(C(F)(F)F)[nH]c1=O]

2-oxo-5-(4-(quinoxalin-6-ylmethoxy)phenyl)-6-(trifluoromethyl)-1,2-dihydropyridine-3-carboxamide (131) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 6-(bromomethyl)quinoxaline. LCMS: Rt=2.93 min, m/z=441.1 (M+H).

### Example 132: 5-(4-((1-methyl-1H-indazol-4-yl)methoxy)phenyl)-2-oxo-6-(trifluoromethyl)-1,2-dihydropyridine-3-carboxamide (132)

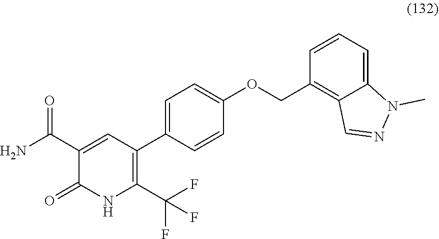 [SMILES: Cn1ncc2c(COc3ccc(-c4cc(C(N)=O)c(=O)[nH]c4C(F)(F)F)cc3)cccc21]

5-(4-((1-methyl-1H-indazol-4-yl)methoxy)phenyl)-2-oxo-6-(trifluoromethyl)-1,2-dihydropyridine-3-carboxamide (132) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)-1-methyl-1H-indazole. LCMS: Rt=3.06 min, m/z=443.1 (M+H).

### Example 133: 5-(4-((4-(methylsulfonyl)benzyl)oxy)phenyl)-2-oxo-6-(trifluoromethyl)-1,2-dihydropyridine-3-carboxamide (133)

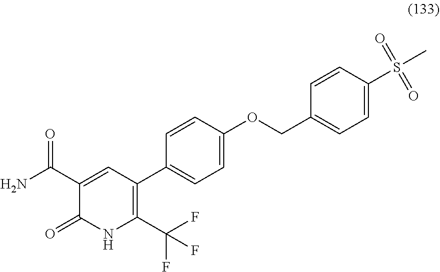 [SMILES: CS(=O)(=O)c1ccc(COc2ccc(-c3cc(C(N)=O)c(=O)[nH]c3C(F)(F)F)cc2)cc1]

5-(4-((4-(methylsulfonyl)benzyl)oxy)phenyl)-2-oxo-6-(trifluoromethyl)-1,2-dihydropyridine-3-carboxamide (133) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 1-(bromomethyl)-4-(methylsulfonyl)benzene. LCMS: Rt=2.85 min, m/z=487.1 (M+H).

### Example 134: 2-oxo-5-(4-(thiazol-5-ylmethoxy)phenyl)-6-(trifluoromethyl)-1,2-dihydropyridine-3-carboxamide (134)

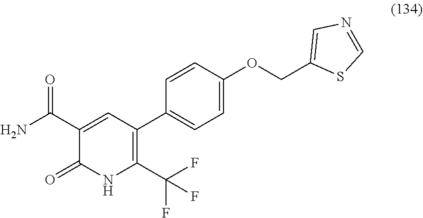 [SMILES: NC(=O)c1cc(-c2ccc(OCc3cncs3)cc2)c(C(F)(F)F)[nH]c1=O]

2-oxo-5-(4-(thiazol-5-ylmethoxy)phenyl)-6-(trifluoromethyl)-1,2-dihydropyridine-3-carboxamide (134) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 5-(bromomethyl)thiazole. LCMS: Rt=2.71 min, m/z=396.1 (M+H).

### Example 135: 5-(4-((4,6-dimethylpyridin-2-ylmethoxy)phenyl)-2-oxo-6-(trifluoromethyl)-1,2-dihydropyridine-3-carboxamide (135)

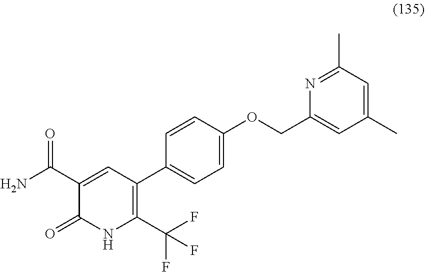 [SMILES: Cc1cc(C)nc(COc2ccc(-c3cc(C(N)=O)c(=O)[nH]c3C(F)(F)F)cc2)c1]

5-(4-((4,6-dimethylpyridin-2-yl)methoxy)phenyl)-2-oxo-6-(trifluoromethyl)-1,2-dihydropyridine-3-carboxamide (135) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 2-(bromomethyl)-4,6-dimethylpyridine. LCMS: Rt=2.30 min, m/z=418.2 (M+H).

### Example 136: 5-(4-((2-methoxypyridin-3-yl)methoxy)phenyl)-2-oxo-6-(trifluoromethyl)-1,2-dihydropyridine-3-carboxamide (136)

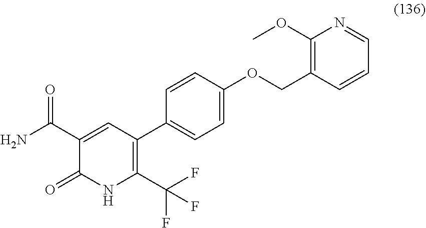 [SMILES: COc1ncccc1COc1ccc(-c2cc(C(N)=O)c(=O)[nH]c2C(F)(F)F)cc1]

5-(4-((2-methoxypyridin-3-yl)methoxy)phenyl)-2-oxo-6-(trifluoromethyl)-1,2-dihydropyridine-3-carboxamide (136) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 3-(bromomethyl)-2-methoxypyridine. LCMS: Rt=3.08 min, m/z=420.1 (M+H).

### Example 137: 5-(4-(isoquinolin-7-ylmethoxy)phenyl)-2-oxo-6-(trifluoromethyl)-1,2-dihydropyridine-3-carboxamide (137)

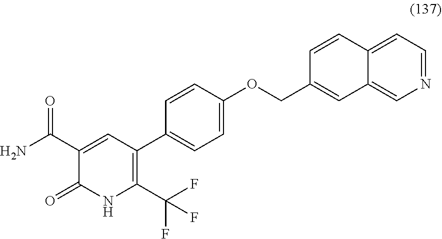 [SMILES: NC(=O)c1cc(-c2ccc(OCc3ccc4ccncc4c3)cc2)c(C(F)(F)F)[nH]c1=O]

5-(4-(isoquinolin-7-ylmethoxy)phenyl)-2-oxo-6-(trifluoromethyl)-1,2-dihydropyridine-3-carboxamide (137) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 7-(bromomethyl)isoquinoline. LCMS: Rt=2.45 min, m/z=440.1 (M+H).

### Example 138: 2-oxo-5-(4-(quinolin-7-ylmethoxy)phenyl)-6-(trifluoromethyl)-1,2-dihydropyridine-3-carboxamide (138)

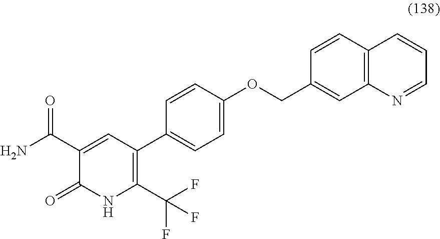 [SMILES: NC(=O)c1cc(-c2ccc(OCc3ccc4cccnc4c3)cc2)c(C(F)(F)F)[nH]c1=O]

2-oxo-5-(4-(quinolin-7-ylmethoxy)phenyl)-6-(trifluoromethyl)-1,2-dihydropyridine-3-carboxamide (138) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 7-(bromomethyl)quinoline. LCMS: Rt=2.44 min, m/z=440.1 (M+H).

### Example 139: 5-(4-((3-fluoropyridin-4-yl)methoxy)phenyl)-2-oxo-6-(trifluoromethyl)-1,2-dihydropyridine-3-carboxamide (139)

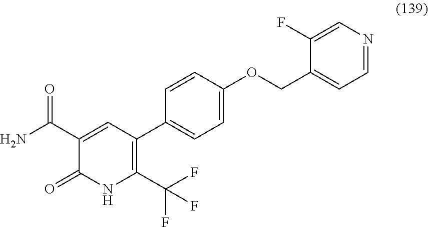 [SMILES: NC(=O)c1cc(-c2ccc(OCc3ccncc3F)cc2)c(C(F)(F)F)[nH]c1=O]

5-(4-((3-fluoropyridin-4-yl)methoxy)phenyl)-2-oxo-6-(trifluoromethyl)-1,2-dihydropyridine-3-carboxamide (139) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 4-(bromomethyl)-3-fluoropyridine. LCMS: Rt=2.76 min, m/z=408.1 (M+H).

### Example 140: 5-(4-((4,6-dimethylpyrimidin-2-yl)methoxy)phenyl)-2-oxo-6-(trifluoromethyl)-1,2-dihydropyridine-3-carboxamide (140)

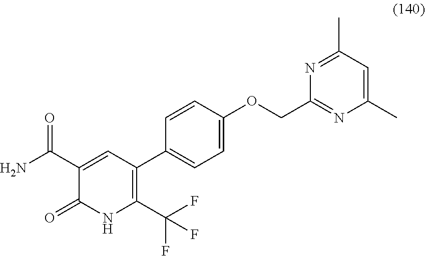 [SMILES: Cc1cc(C)nc(COc2ccc(-c3cc(C(N)=O)c(=O)[nH]c3C(F)(F)F)cc2)n1]

5-(4-((4,6-dimethylpyrimidin-2-yl)methoxy)phenyl)-2-oxo-6-(trifluoromethyl)-1,2-dihydropyridine-3-carboxamide (140) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 2-(bromomethyl)-4,6-dimethylpyrimidine. LCMS: Rt=2.67 min, m/z=419.1 (M+H).

### Example 141: 5-(4-((1-methyl-1H-imidazol-2-yl)methoxy)phenyl)-2-oxo-6-(trifluoromethyl)-1,2-dihydropyridine-3-carboxamide (141)

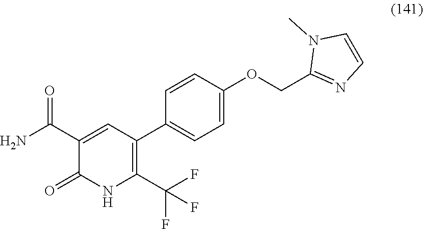 [SMILES: Cn1ccnc1COc1ccc(-c2cc(C(N)=O)c(=O)[nH]c2C(F)(F)F)cc1]

5-(4-((1-methyl-1H-imidazol-2-yl)methoxy)phenyl)-2-oxo-6-(trifluoromethyl)-1,2-dihydropyridine-3-carboxamide (141) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 2-(bromomethyl)-1-methyl-1H-imidazole. LCMS: Rt=2.10 min, m/z=393.1 (M+H).

### Example 142: 5-(4-((2-morpholinopyridin-3-yl)methoxy)phenyl)-2-oxo-6-(trifluoromethyl)-1,2-dihydropyridine-3-carboxamide (142)

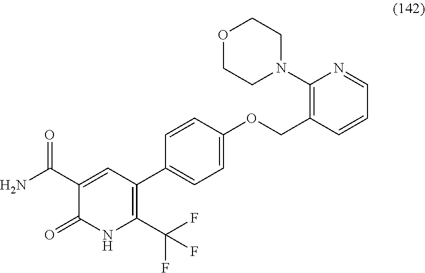 [SMILES: NC(=O)c1cc(-c2ccc(OCc3cccnc3N3CCOCC3)cc2)c(C(F)(F)F)[nH]c1=O]

5-(4-((2-morpholinopyridin-3-yl)methoxy)phenyl)-2-oxo-6-(trifluoromethyl)-1,2-dihydropyridine-3-carboxamide (142) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(3-(bromomethyl)pyridin-2-yl)morpholine. LCMS: Rt=2.56 min, m/z=475.1 (M+H).

### Example 143: 2-oxo-5-(4-(thiazol-2-ylmethoxy)phenyl)-6-(trifluoromethyl)-1,2-dihydropyridine-3-carboxamide 143

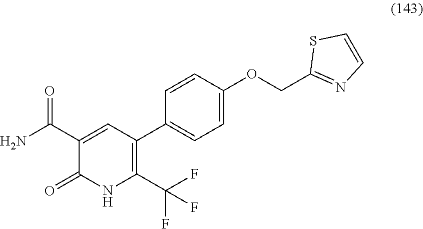 [SMILES: NC(=O)c1cc(-c2ccc(OCc3nccs3)cc2)c(C(F)(F)F)[nH]c1=O]

2-oxo-5-(4-(thiazol-2-ylmethoxy)phenyl)-6-(trifluoromethyl)-1,2-dihydropyridine-3-carboxamide (143) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 2-(bromomethyl)thiazole. LCMS: Rt=2.82 min, m/z=396.1 (M+H).

### Example 144: 5-(4-((3,4-dimethoxypyridin-2-yl)methoxy)phenyl)-2-oxo-6-(trifluoromethyl)-1,2-dihydropyridine-3-carboxamide (144)

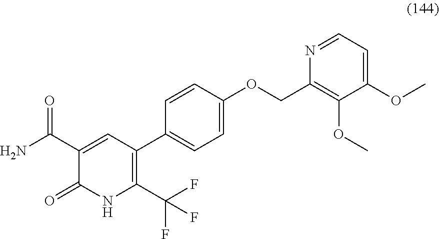 [SMILES: COc1ccnc(COc2ccc(-c3cc(C(N)=O)c(=O)[nH]c3C(F)(F)F)cc2)c1OC]

5-(4-((3,4-dimethoxypyridin-2-yl)methoxy)phenyl)-2-oxo-6-(trifluoromethyl)-1,2-dihydropyridine-3-carboxamide (144) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 2-(bromomethyl)-3,4-dimethoxypyridine. LCMS: Rt=2.31 min, m/z=450.1 (M+H).

### Example 145: 5-(4-((1-methyl-1H-indazol-5-yl)methoxy)phenyl)-2-oxo-6-(trifluoromethyl)-1,2-dihydropyridine-3-carboxamide (145)

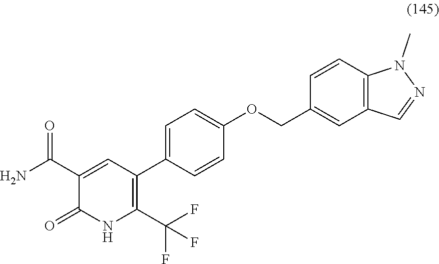

5-(4-((1-methyl-1H-indazol-5-yl)methoxy)phenyl)-2-oxo-6-(trifluoromethyl)-1,2-dihydropyridine-3-carboxamide (145) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(bromomethyl)-1-methyl-1H-indazole. LCMS: Rt=3.06 min, m/z=443.1 (M+H).

### Example 146: 5-(4-((5-methoxypyridin-3-yl)methoxy)phenyl)-2-oxo-6-(trifluoromethyl)-1,2-dihydropyridine-3-carboxamide (146)

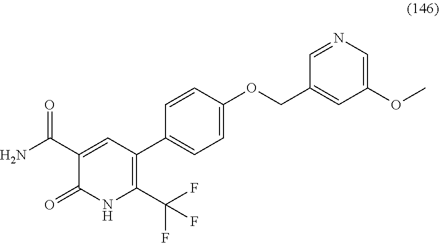 [SMILES: COc1cncc(COc2ccc(-c3cc(C(N)=O)c(=O)[nH]c3C(F)(F)F)cc2)c1]

5-(4-((5-methoxypyridin-3-yl)methoxy)phenyl)-2-oxo-6-(trifluoromethyl)-1,2-dihydropyridine-3-carboxamide (146) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 3-(bromomethyl)-5-methoxypyridine. LCMS: Rt=2.37 min, m/z=420.1 (M+H).

### Example 147: 5-(4-((2,6-dimethylpyridin-4-yl)methoxy)phenyl)-2-oxo-6-(trifluoromethyl)-1,2-dihydropyridine-3-carboxamide (147)

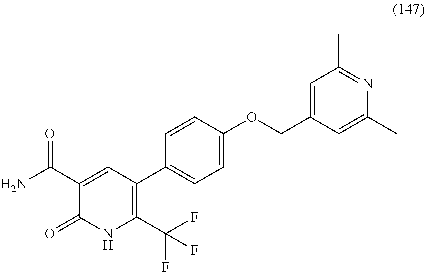 [SMILES: Cc1cc(COc2ccc(-c3cc(C(N)=O)c(=O)[nH]c3C(F)(F)F)cc2)cc(C)n1]

5-(4-((2,6-dimethylpyridin-4-yl)methoxy)phenyl)-2-oxo-6-(trifluoromethyl)-1,2-dihydropyridine-3-carboxamide (147) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)-2,6-dimethylpyridine. LCMS: Rt=2.32 min, m/z=418.1 (M+H).

### Example 148: 5-(4-((6-chlorobenzo[d][1,3]dioxol-5-yl)methoxy)phenyl)-2-oxo-6-(trifluoromethyl)-1,2-dihydropyridine-3-carboxamide (148)

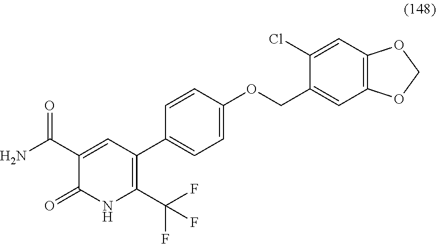 [SMILES: NC(=O)c1cc(-c2ccc(OCc3cc4c(cc3Cl)OCO4)cc2)c(C(F)(F)F)[nH]c1=O]

5-(4-((6-chlorobenzo[d][1,3]dioxol-5-yl)methoxy)phenyl)-2-oxo-6-(trifluoromethyl)-1,2-dihydropyridine-3-carboxamide (148) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(bromomethyl)-6-chlorobenzo[d][1,3]dioxole. LCMS: Rt=3.03 min, m/z=467.1 (M+H).

### Example 149: 5-(4-((3,4-dihydro-2H-benzo[b][1,4]dioxepin-7-yl)methoxy)phenyl)-2-oxo-6-(trifluoromethyl)-1,2-dihydropyridine-3-carboxamide (149)

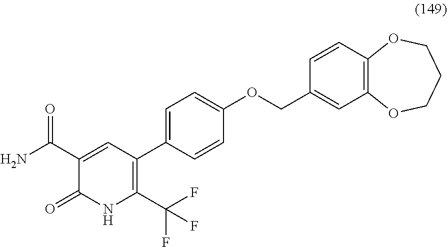 [SMILES: NC(=O)c1cc(-c2ccc(OCc3ccc4c(c3)OCCCO4)cc2)c(C(F)(F)F)[nH]c1=O]

5-(4-((3,4-dihydro-2H-benzo[b][1,4]dioxepin-7-yl)methoxy)phenyl)-2-oxo-6-(trifluoromethyl)-1,2-dihydropyridine-3-carboxamide (149) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 7-(bromomethyl)-3,4-dihydro-2H-benzo[b][1,4]dioxepine. LCMS: Rt=1.80 min, m/z=461.1 (M+H).

### Example 150: 5-(4-((2-hydroxypyrimidin-5-yl)methoxy)phenyl)-2-oxo-6-(trifluoromethyl)-1,2-dihydropyridine-3-carboxamide (150)

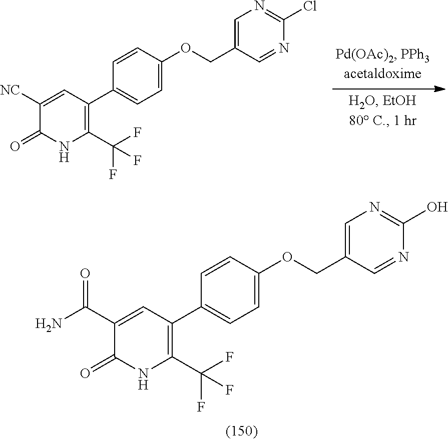 [SMILES: N#Cc1cc(-c2ccc(OCc3cnc(Cl)nc3)cc2)c(C(F)(F)F)[nH]c1=O ; NC(=O)c1cc(-c2ccc(OCc3cnc(O)nc3)cc2)c(C(F)(F)F)[nH]c1=O]

To a solution of 5-(4-((2-chloropyrimidin-5-yl)methoxy)phenyl)-2-oxo-6-(trifluoromethyl)-1,2-dihydropyridine-3-carbonitrile (50 mg, 0.12 mmol), acetaldoxime (14.5 mg, 0.25 mmol), PPh₃ (0.48 mg, 0.02 mmol) in EtOH (1 mL) and H₂O (0.5 mL) was added Pd(OAc)₂ (2 mg, 0.01 mmol) at 25° C. The mixture was stirred at 80° C. for 1 hr. The reaction mixture was filtered and purified by prep-HPLC (acid) to give 5-(4-((2-hydroxypyrimidin-5-yl)methoxy)phenyl)-2-oxo-6-(trifluoromethyl)-1,2-dihydropyridine-3-carboxamide (150). ¹H NMR (400 MHz, DMSO) δ=10.18 (br s, 1H) 8.43 (s, 2H), 8.25 (s, 1H), 7.91 (s, 1H), 7.32-7.31 (m, 1H), 7.19 (d, J=8.4 Hz, 2H), 7.03 (d, J=8.4 Hz, 2H), 4.88 (s, 2H). LCMS: Rt=0.56 min, m/z=406.9 (M+H).

5-(4-((2-chloropyrimidin-5-yl)methoxy)phenyl)-2-oxo-6-(trifluoromethyl)-1,2-dihydropyridine-3-carbonitrile was obtained using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(bromomethyl)-2-chloropyrimidine, and 5-bromo-2-oxo-6-(trifluoromethyl)-1,2-dihydropyridine-3-carboxamide was replaced with 5-bromo-2-oxo-6-(trifluoromethyl)-1,2-dihydropyridine-3-carbonitrile. LCMS: Rt=0.80 min, m/z=406.8 (M+H).

### Example 151: 5-(4-((2-(oxetan-3-ylmethoxy)pyrimidin-5-yl)methoxy)phenyl)-2-oxo-6-(trifluoromethyl)-1,2-dihydropyridine-3-carboxamide (151)

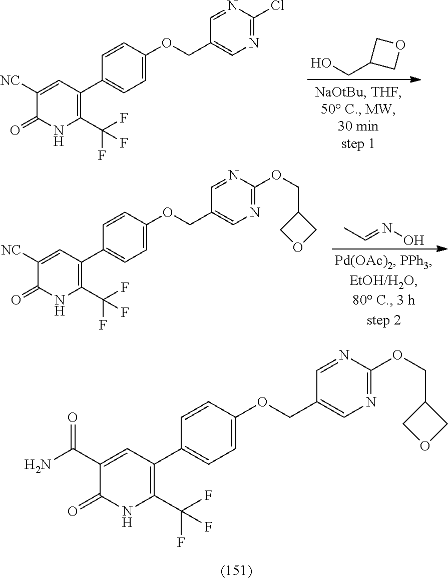

Step 1: To a solution of oxetan-3-ylmethanol (163 mg, 1.84 mmol) in THF (1 mL) was added tBuONa (53 mg, 0.55 mmol) and the mixture was stirred at 25° C. for 15 min. 5-(4-((2-chloropyrimidin-5-yl)methoxy)phenyl)-2-oxo-6-(trifluoromethyl)-1,2-dihydropyridine-3-carbonitrile (150 mg, 0.37 mmol) in THF (1 mL) was then added via syringe and the mixture stirred at 50° C. for 10 min under microwave. The reaction mixture was poured into water (10 mL) and extracted with chloroform/isopropanol=3/1 (10 mL×2). The organic layers were combined and dried over anhydrous Na₂SO₄, filtered and concentrated to give crude 5-(4-((2-(oxetan-3-ylmethoxy)pyrimidin-5-yl)methoxy)phenyl)-2-oxo-6-(trifluoromethyl)-1,2-dihydropyridine-3-carbonitrile which was used for next step without further purification. LCMS: Rt=0.90 min, m/z=459.1 [M+H].

Step 2: To a solution of 5-(4-((2-(oxetan-3-ylmethoxy)pyrimidin-5-yl)methoxy)phenyl)-2-oxo-6-(trifluoromethyl)-1,2-dihydropyridine-3-carbonitrile (0.2 g, 0.44 mmol), acetaldoxime (52 mg, 0.87 mmol), PPh₃ (23 mg, 0.08 mmol) in the mixture of EtOH (3 mL) and H2O (1 mL) was added Pd(OAc)₂ (10 mg, 0.04 mmol) at 25° C. under N₂. The mixture was heated to 80° C. and stirred for 3 hr. The reaction mixture was filtered, concentrated and purified by prep-HPLC (base) to give 5-(4-((2-(oxetan-3-ylmethoxy)pyrimidin-5-yl)methoxy)phenyl)-2-oxo-6-(trifluoromethyl)-1,2-dihydropyridine-3-carboxamide (151). ¹H NMR (400 MHz, DMSO-d6) δ 10.75 (br. s, 1H), 8.74 (s, 2H), 7.80 (s, 1H), 7.16 (d, J=8.8 Hz, 2H), 7.02 (d, J=8.8 Hz, 2H), 5.08 (s, 2H), 4.72 (m, 2H), 4.55 (m, 2H), 4.45 (m, 2H); LCMS: Rt=0.70 min, m/z=477.1 [M+H].

### Example 152: 5-(4-((2-(2-hydroxyethoxy)pyrimidin-5-yl)methoxy)phenyl)-2-oxo-6-(trifluoromethyl)-1,2-dihydropyridine-3-carboxamide (152)

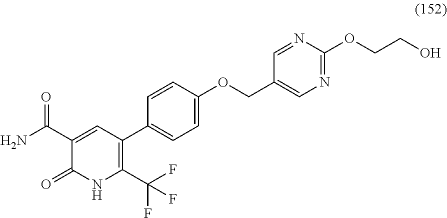 [SMILES: NC(=O)c1cc(-c2ccc(OCc3cnc(OCCO)nc3)cc2)c(C(F)(F)F)[nH]c1=O]

5-(4-((2-(2-hydroxyethoxy)pyrimidin-5-yl)methoxy)phenyl)-2-oxo-6-(trifluoromethyl)-1,2-dihydropyridine-3-carboxamide (152) was made using a procedure similar to that used to make 5-(4-((2-(oxetan-3-ylmethoxy)pyrimidin-5-yl)methoxy)phenyl)-2-oxo-6-(trifluoromethyl)-1,2-dihydropyridine-3-carboxamide (151), except oxetan-3-ylmethanol was replaced with ethane-1,2-diol. ¹H NMR (400 MHz, DMSO-d6) δ 9.61 (br., s, 1H), 8.73 (s, 2H), 8.24 (br., s, 1H), 8.02 (s, 1H), 7.57 (br., s, 1H), 7.22 (d, J=7.6 Hz, 2H), 7.08 (d, J=7.6 Hz, 2H), 5.10 (s, 2H), 4.34 (s, 2H), 3.73 (s, 1H); LCMS: Rt=0.69 min, m/z=451.0 [M+H].

### Example 153: 2-oxo-5-(4-((2-propoxypyrimidin-5-yl)methoxy)phenyl)-6-(trifluoromethyl)-1,2-dihydropyridine-3-carboxamide (153)

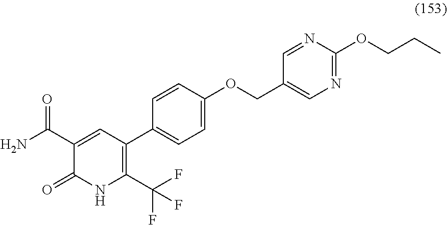 [SMILES: CCCOc1ncc(COc2ccc(-c3cc(C(N)=O)c(=O)[nH]c3C(F)(F)F)cc2)cn1]

2-oxo-5-(4-((2-propoxypyrimidin-5-yl)methoxy)phenyl)-6-(trifluoromethyl)-1,2-dihydropyridine-3-carboxamide (153) was made using a procedure similar to that used to make 5-(4-((2-(oxetan-3-ylmethoxy)pyrimidin-5-yl)methoxy)phenyl)-2-oxo-6-(trifluoromethyl)-1,2-dihydropyridine-3-carboxamide (151), except oxetan-3-ylmethanol was replaced with propan-1-ol. ¹H NMR (400 MHz, DMSO-d6) δ 9.49 (br. s, 1H), 8.73 (s, 2H), 8.19 (s, 1H), 8.05 (s, 1H), 7.64 (br.s, 1H), 7.24 (d, J=8.4 Hz, 2H), 7.08 (d, J=8.4 Hz, 2H), 5.10 (s, 2H), 4.29 (m, 2H), 1.77 (m, 2H), 0.99 (m, 3H); LCMS: Rt=0.80 min, m/z=449.1 [M+H].

### Example 154: 2-oxo-5-(4-((2-((tetrahydrofuran-3-yl)oxy)pyrimidin-5-yl)methoxy)phenyl)-6-(trifluoromethyl)-1,2-dihydropyridine-3-carboxamide (154)

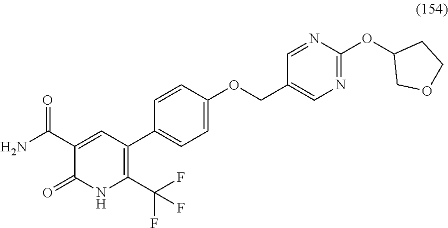 [SMILES: NC(=O)c1cc(-c2ccc(OCc3cnc(OC4CCOC4)nc3)cc2)c(C(F)(F)F)[nH]c1=O]

2-oxo-5-(4-((2-((tetrahydrofuran-3-yl)oxy)pyrimidin-5-yl)methoxy)phenyl)-6-(trifluoromethyl)-1,2-dihydropyridine-3-carboxamide (154) was made using a procedure similar to that used to make 5-(4-((2-(oxetan-3-ylmethoxy)pyrimidin-5-yl)methoxy)phenyl)-2-oxo-6-(trifluoromethyl)-1,2-dihydropyridine-3-carboxamide (151), except oxetan-3-ylmethanol was replaced with tetrahydrofuran-3-ol. ¹H NMR (400 MHz, DMSO-d6) δ 8.75 (s, 2H), 8.22 (s, 1H), 8.02 (s, 1H), 7.29 (d, J=8.4 Hz, 2H), 7.13 (d, J=8.4 Hz, 2H), 5.52 (m, 1H), 5.13 (s, 2H), 3.94-3.79 (m, 4H), 2.27 (m, 1H), 2.06 (m, 1H); LCMS: Rt=0.66 min, m/z=477.1 [M+H].

### Example 155: 5-(4-((2-(cyclopentyloxy)pyrimidin-5-yl)methoxy)phenyl)-2-oxo-6-(trifluoromethyl)-1,2-dihydropyridine-3-carboxamide (155)

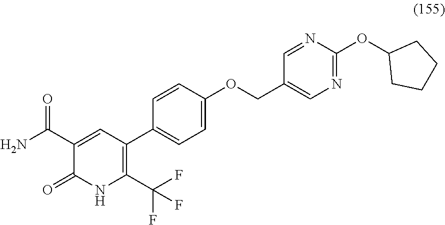 [SMILES: NC(=O)c1cc(-c2ccc(OCc3cnc(OC4CCCC4)nc3)cc2)c(C(F)(F)F)[nH]c1=O]

5-(4-((2-(cyclopentyloxy)pyrimidin-5-yl)methoxy)phenyl)-2-oxo-6-(trifluoromethyl)-1,2-dihydropyridine-3-carboxamide (155) was made using a procedure similar to that used to make 5-(4-((2-(oxetan-3-ylmethoxy)pyrimidin-5-yl)methoxy)phenyl)-2-oxo-6-(trifluoromethyl)-1,2-dihydropyridine-3-carboxamide (151), except oxetan-3-ylmethanol was replaced with cyclopentanol. ¹H NMR (400 MHz, DMSO-d6) δ 10.70 (br., s, 1H), 8.71 (s, 2H), 7.82 (s, 1H), 7.17 (d, J=8.4 Hz, 2H), 7.04 (d, J=8.8 Hz, 2H), 5.39 (m, 2H), 5.07 (s, 2H), 1.99 (m, 2H), 1.76-1.71 (m, 4H), 1.61 (m, 2H); LCMS: Rt=0.91 min, m/z=497.1 [M+H].

### Example 156: 5-(4-((2-(oxetan-2-ylmethoxy)pyrimidin-5-yl)methoxy)phenyl)-2-oxo-6-(trifluoromethyl)-1,2-dihydropyridine-3-carboxamide (156)

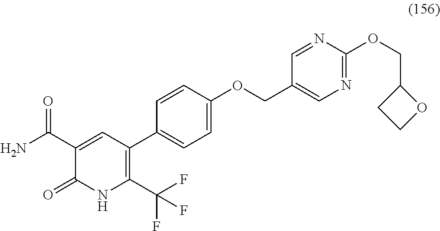 [SMILES: NC(=O)c1cc(-c2ccc(OCc3cnc(OCC4CCO4)nc3)cc2)c(C(F)(F)F)[nH]c1=O]

5-(4-((2-(oxetan-2-ylmethoxy)pyrimidin-5-yl)methoxy)phenyl)-2-oxo-6-(trifluoromethyl)-1,2-dihydropyridine-3-carboxamide (156) was made using a procedure similar to that used to make 5-(4-((2-(oxetan-3-ylmethoxy)pyrimidin-5-yl)methoxy)phenyl)-2-oxo-6-(trifluoromethyl)-1,2-dihydropyridine-3-carboxamide (151), except oxetan-3-ylmethanol was replaced with oxetan-2-ylmethanol. ¹H NMR (400 MHz, DMSO-d6) δ 10.75 (s, 1H), 8.74 (s, 2H), 7.80 (s, 1H), 7.16 (d, J=8.8 Hz, 2H), 7.02 (d, J=8.8 Hz, 2H), 5.08 (s, 2H), 4.72 (m, 2H), 4.55 (m, 2H), 4.45 (m, 2H); LCMS: Rt=0.70 min, m/z=477.1 [M+H].

### Example 157: 2-oxo-5-(4-((2-((tetrahydrofuran-3-yl)methoxy)pyrimidin-5-yl)methoxy)phenyl)-6-(trifluoromethyl)-1,2-dihydropyridine-3-carboxamide (157)

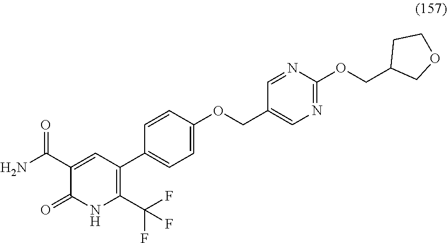 [SMILES: NC(=O)c1cc(-c2ccc(OCc3cnc(OCC4CCOC4)nc3)cc2)c(C(F)(F)F)[nH]c1=O]

2-oxo-5-(4-((2-((tetrahydrofuran-3-yl)methoxy)pyrimidin-5-yl)methoxy)phenyl)-6-(trifluoromethyl)-1,2-dihydropyridine-3-carboxamide (157) was made using a procedure similar to that used to make 5-(4-((2-(oxetan-3-ylmethoxy)pyrimidin-5-yl)methoxy)phenyl)-2-oxo-6-(trifluoromethyl)-1,2-dihydropyridine-3-carboxamide (151), except oxetan-3-ylmethanol was replaced with (tetrahydrofuran-3-yl)methanol. ¹H NMR (400 MHz, DMSO-d6) δ 8.96 (br. s, 1H), 8.74 (s, 2H), 8.17 (br. s, 1H), 7.96 (s, 1H), 7.28 (d, J=8.4 Hz, 2H), 7.11 (d, J=8.4 Hz, 2H), 5.12 (s, 2H), 4.30-4.23 (m, 2H), 3.81-3.76 (m, 2H), 3.66-3.60 (m, 2H), 2.71 (m, 1H), 2.03 (m, 1H), 1.68 (m, 1H); LCMS: Rt=0.68 min, m/z=491.1 [M+H].

### Example 158: 5-(4-((2-(methylamino)pyrimidin-5-yl)methoxy)phenyl)-2-oxo-6-(trifluoromethyl)-1,2-dihydropyridine-3-carboxamide (158)

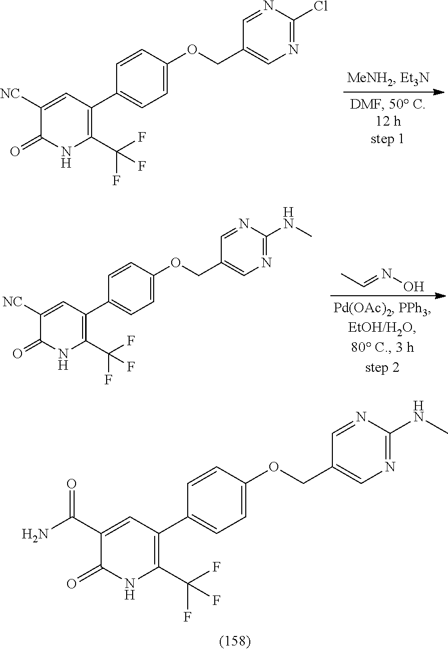

Step 1: To a solution of 5-(4-((2-chloropyrimidin-5-yl)methoxy)phenyl)-2-oxo-6-(trifluoromethyl)-1,2-dihydropyridine-3-carbonitrile (80 mg, 0.2 mmol) in DMF (1 mL) was added methanamine (8 mg, 0.24 mmol) and Et₃N (40 mg, 0.4 mmol) at 25° C. The mixture was stirred at 50° C. for 12 hrs. The mixture was washed with H₂O (1 mL) and extracted with EA (1 mL×2). The combined organic layers were dried over anhydrous Na₂SO₄, filtered and concentrated. The residue was purified by reverse phase prep-HPLC to give 5-(4-((2-(methylamino)pyrimidin-5-yl)methoxy)phenyl)-2-oxo-6-(trifluoromethyl)-1,2-dihydropyridine-3-carbonitrile. LCMS: Rt=0.71 min, m/z=402.0 [M+H].

Step 2: To a solution of 5-(4-((2-(methylamino)pyrimidin-5-yl)methoxy)phenyl)-2-oxo-6-(trifluoromethyl)-1,2-dihydropyridine-3-carbonitrile (0.18 g, 0.44 mmol), acetaldoxime (52 mg, 0.87 mmol), PPh₃ (23 mg, 0.08 mmol) in the mixture of EtOH (3 mL) and H2O (1 mL) was added Pd(OAc)₂ (10 mg, 0.04 mmol) at 25° C. under N₂. The mixture was heated to 80° C. and stirred for 3 hr. The reaction mixture was filtered, concentrated and purified by prep-HPLC (base) to give 5-(4-((2-(methylamino)pyrimidin-5-yl)methoxy)phenyl)-2-oxo-6-(trifluoromethyl)-1,2-dihydropyridine-3-carboxamide (158). ¹H NMR (400 MHz, DMSO-d6) δ 10.20 (br. s, 1H), 8.40 (s, 2H), 8.29 (br. s, 1H), 8.32-8.26 (m, 1H), 7.89 (s, 1H), 7.16 (d, J=8.4 Hz, 2H), 7.01 (d, J=8.4 Hz, 2H), 4.92 (s, 2H), 2.81 (d, J=4.8 Hz, 3H); LCMS: Rt=0.62 min, m/z=420.1 [M+H].

### Example 159: 5-(4-((2-(cyclopentylamino)pyrimidin-5-yl)methoxy)phenyl)-2-oxo-6-(trifluoromethyl)-1,2-dihydropyridine-3-carboxamide (159)

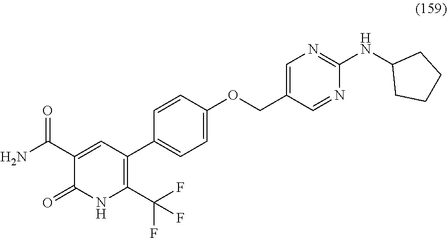 [SMILES: NC(=O)c1cc(-c2ccc(OCc3cnc(NC4CCCC4)nc3)cc2)c(C(F)(F)F)[nH]c1=O]

5-(4-((2-(cyclopentylamino)pyrimidin-5-yl)methoxy)phenyl)-2-oxo-6-(trifluoromethyl)-1,2-dihydropyridine-3-carboxamide (159) was made using a procedure similar to that used to make 5-(4-((2-(methylamino)pyrimidin-5-yl)methoxy)phenyl)-2-oxo-6-(trifluoromethyl)-1,2-dihydropyridine-3-carboxamide (158), except methanamine was replaced with cyclopentanamine. ¹H NMR (400 MHz, DMSO) δ=8.41 (s, 2H), 8.39 (m, 1H), 7.31 (d, J=7.6 Hz, 1H), 7.25 (d, J=8.0 Hz, 2H), 7.07 (d, J=8.4 Hz, 2H), 4.93 (s, 2H), 4.19-4.14 (m, 1H), 1.90-1.88 (m, 2H), 1.68-1.49 (m, 2H), 1.15-0.87 (m, 5H). LCMS: Rt=0.76 min, m/z=474.0 (M+H).

### Example 160: 5-(4-((2-(dimethylamino)pyrimidin-5-yl)methoxy)phenyl)-2-oxo-6-(trifluoromethyl)-1,2-dihydropyridine-3-carboxamide (160)

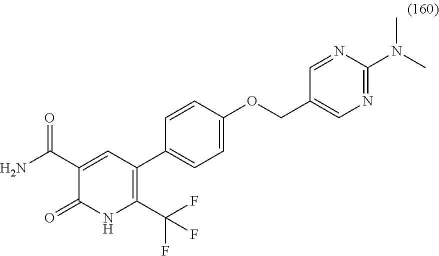 [SMILES: CN(C)c1ncc(COc2ccc(-c3cc(C(N)=O)c(=O)[nH]c3C(F)(F)F)cc2)cn1]

5-(4-((2-(dimethylamino)pyrimidin-5-yl)methoxy)phenyl)-2-oxo-6-(trifluoromethyl)-1,2-dihydropyridine-3-carboxamide (160) was made using a procedure similar to that used to make 5-(4-((2-(methylamino)pyrimidin-5-yl)methoxy)phenyl)-2-oxo-6-(trifluoromethyl)-1,2-dihydropyridine-3-carboxamide (158), except methanamine was replaced with dimethylamine. ¹H NMR (400 MHz, DMSO-d6) δ 8.47 (s, 2H), 8.27 (m, 1H), 7.88 (s, 1H), 7.16 (d, J=8.4 Hz, 2H), 7.01 (d, J=8.4 Hz, 2H), 4.94 (s, 2H), 3.13 (s, 6H); LCMS: Rt=0.75 min, m/z=434.0 [M+H].

### Example 161: 5-(4-((2-((cyclopropylmethylamino)pyrimidin-5-yl)methoxy)phenyl)-2-oxo-6-(trifluoromethyl)-1,2-dihydropyridine-3-carboxamide (161)

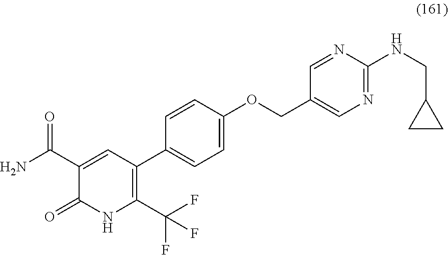 [SMILES: NC(=O)c1cc(-c2ccc(OCc3cnc(NCC4CC4)nc3)cc2)c(C(F)(F)F)[nH]c1=O]

5-(4-((2-((cyclopropylmethyl)amino)pyrimidin-5-yl)methoxy)phenyl)-2-oxo-6-(trifluoromethyl)-1,2-dihydropyridine-3-carboxamide (161) was made using a procedure similar to that used to make 5-(4-((2-(methylamino)pyrimidin-5-yl)methoxy)phenyl)-2-oxo-6-(trifluoromethyl)-1,2-dihydropyridine-3-carboxamide (158), except methanamine was replaced with cyclopropylmethanamine. ¹H NMR (400 MHz, DMSO-d6) δ 9.83 (br. s, 1H), 8.17 (br. s, 2H), 8.02 (s, 1H), 7.73 (br. s, 1H), 7.25-7.08 (m, 2H), 7.04-6.88 (m, 2H), 6.96 (d, J=8.0 Hz, 1H), 6.81 (d, J=8.0 Hz, 1H), 4.79-4.62 (m, 2H), 2.95 (s, 2H), 0.85 (s, 1H), 0.19 (d, J=5.9 Hz, 2H), 0.00 (s, 2H); LCMS: Rt=0.78 min, m/z=460.0 [M+H].

### Example 162: 5-(4-((2-((2-hydroxyethyl)amino)pyrimidin-5-yl)methoxy)phenyl)-2-oxo-6-(trifluoromethyl)-1,2-dihydropyridine-3-carboxamide (162)

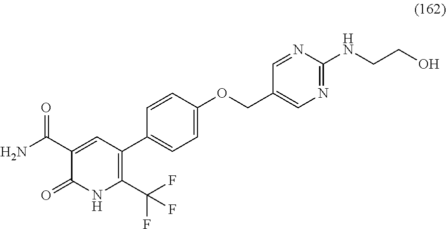 [SMILES: NC(=O)c1cc(-c2ccc(OCc3cnc(NCCO)nc3)cc2)c(C(F)(F)F)[nH]c1=O]

5-(4-((2-((2-hydroxyethyl)amino)pyrimidin-5-yl)methoxy)phenyl)-2-oxo-6-(trifluoromethyl)-1,2-dihydropyridine-3-carboxamide (162) was made using a procedure similar to that used to make 5-(4-((2-(methylamino)pyrimidin-5-yl)methoxy)phenyl)-2-oxo-6-(trifluoromethyl)-1,2-dihydropyridine-3-carboxamide (158), except methanamine was replaced with 2-aminoethan-1-ol. ¹H NMR (400 MHz, DMSO-d6) δ 8.40 (s, 2H), 8.07 (br. s, 1H), 7.66 (br. s, 1H), 7.23 (m, 1H), 7.16 (m, 2H), 4.93 (s, 2H), 4.68 (br.s, 1H), 3.51 (br.s, 2H), 3.38 (m, 2H); LCMS: Rt=0.62 min, m/z=450.1 [M+H].

### Example 163: 5-(4-((2-(cyclopropylamino)pyrimidin-5-yl)methoxy)phenyl)-2-oxo-6-(trifluoromethyl)-1,2-dihydropyridine-3-carboxamide (163)

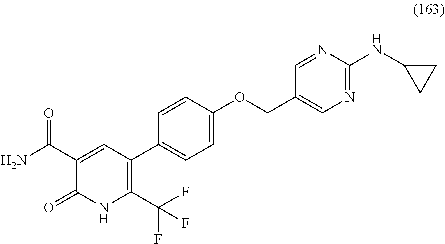 [SMILES: NC(=O)c1cc(-c2ccc(OCc3cnc(NC4CC4)nc3)cc2)c(C(F)(F)F)[nH]c1=O]

5-(4-((2-(cyclopropylamino)pyrimidin-5-yl)methoxy)phenyl)-2-oxo-6-(trifluoromethyl)-1,2-dihydropyridine-3-carboxamide (163) was made using a procedure similar to that used to make 5-(4-((2-(methylamino)pyrimidin-5-yl)methoxy)phenyl)-2-oxo-6-(trifluoromethyl)-1,2-dihydropyridine-3-carboxamide (158), except methanamine was replaced with cyclopropanamine. LCMS: Rt=0.64 min, m/z=448.1 [M+H].

### Example 164: 5-(4-((2-(heptylamino)pyrimidin-5-yl)methoxy)phenyl)-2-oxo-6-(trifluoromethyl)-1,2-dihydropyridine-3-carboxamide (164)

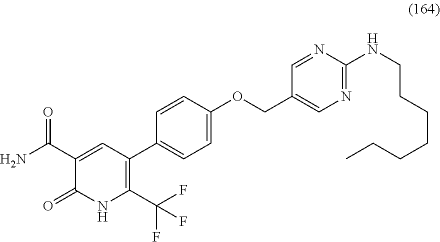 [SMILES: CCCCCCCNc1ncc(COc2ccc(-c3cc(C(N)=O)c(=O)[nH]c3C(F)(F)F)cc2)cn1]

5-(4-((2-(heptylamino)pyrimidin-5-yl)methoxy)phenyl)-2-oxo-6-(trifluoromethyl)-1,2-dihydropyridine-3-carboxamide (164) was made using a procedure similar to that used to make 5-(4-((2-(methylamino)pyrimidin-5-yl)methoxy)phenyl)-2-oxo-6-(trifluoromethyl)-1,2-dihydropyridine-3-carboxamide (158), except methanamine was replaced with heptan-1-amine. LCMS: Rt=0.96 min, m/z=504.4 [M+H].

### Example 165: 5-(4-((2-(oxetan-3-ylamino)pyrimidin-5-yl)methoxy)phenyl)-2-oxo-6-(trifluoromethyl)-1,2-dihydropyridine-3-carboxamide (165)

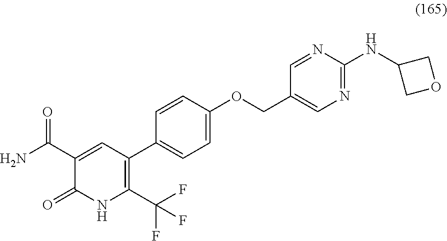 [SMILES: NC(=O)c1cc(-c2ccc(OCc3cnc(NC4COC4)nc3)cc2)c(C(F)(F)F)[nH]c1=O]

5-(4-((2-(oxetan-3-ylamino)pyrimidin-5-yl)methoxy)phenyl)-2-oxo-6-(trifluoromethyl)-1,2-dihydropyridine-3-carboxamide (165) was made using a procedure similar to that used to make 5-(4-((2-(methylamino)pyrimidin-5-yl)methoxy)phenyl)-2-oxo-6-(trifluoromethyl)-1,2-dihydropyridine-3-carboxamide (158), except methanamine was replaced with oxetan-3-amine. ¹H NMR (400 MHz, DMSO-d6) δ=10.54 (br. s, 1H), 8.42 (s, 2H), 8.03 (d, J=5.8 Hz, 1H), 7.83 (s, 1H), 7.15 (d, J=8.6 Hz, 2H), 7.00 (d, J=8.6 Hz, 2H), 4.92 (s, 2H), 4.77 (t, J=6.4 Hz, 2H), 4.51 (t, J=6.4 Hz, 2H), 1.23 (s, 1H); LCMS: Rt=0.63 min, m/z=462.1 [M+H].

### Example 166: 2-oxo-5-(4-((2-((tetrahydrofuran-3-yl)amino)pyrimidin-5-yl)methoxy)phenyl)-6-(trifluoromethyl)-1,2-dihydropyridine-3-carboxamide (166)

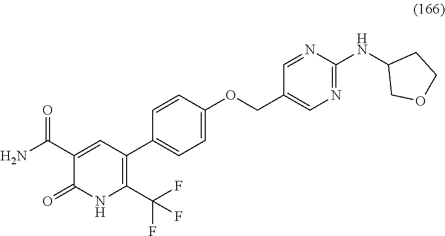 [SMILES: NC(=O)c1cc(-c2ccc(OCc3cnc(NC4CCOC4)nc3)cc2)c(C(F)(F)F)[nH]c1=O]

2-oxo-5-(4-((2-((tetrahydrofuran-3-yl)amino)pyrimidin-5-yl)methoxy)phenyl)-6-(trifluoromethyl)-1,2-dihydropyridine-3-carboxamide (166) was made using a procedure similar to that used to make 5-(4-((2-(methylamino)pyrimidin-5-yl)methoxy)phenyl)-2-oxo-6-(trifluoromethyl)-1,2-dihydropyridine-3-carboxamide (158), except methanamine was replaced with tetrahydrofuran-3-amine. ¹H NMR (400 MHz, DMSO-d6) δ=8.42 (s, 2H), 7.94 (s, 1H), 7.53 (d, J=6.4 Hz, 1H), 7.18 (d, J=8.2 Hz, 2H), 7.03 (d, J=8.8 Hz, 2H), 4.93 (s, 2H), 4.39 (d, J=7.2 Hz, 1H), 3.91-3.82 (m, 3H), 3.76-3.67 (m, 2H), 3.55 (d, J=8.6 Hz, 1H), 2.18-2.11 (m, 1H), 1.88 (t, J=12.4 Hz, 1H); LCMS: Rt=0.64 min, m/z=476.1 [M+H].

### Example 167: 5-(4-((6-(cyclopentylamino)pyridin-3-yl)methoxy)phenyl)-2-oxo-6-(trifluoromethyl)-1,2-dihydropyridine-3-carboxamide (167)

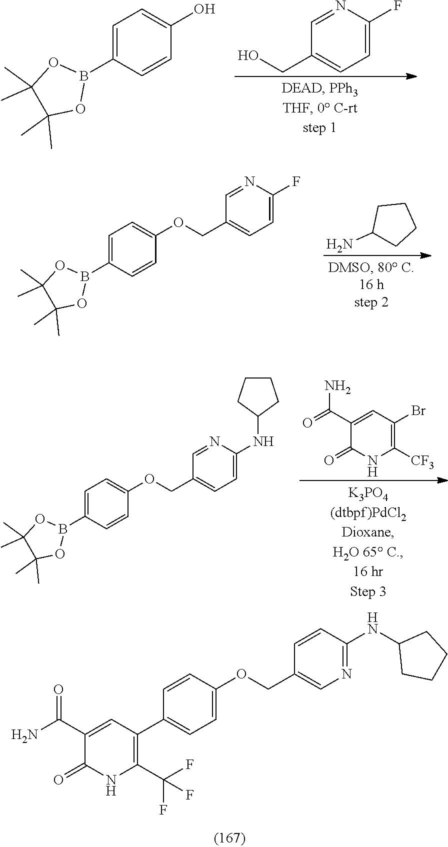

Step 1: (6-fluoropyridin-3-yl)methanol (300 mg, 2.36 mmol), 4-(4,4,5,5-tetramethyl-1,3,2-dioxaborolan-2-yl)phenol (623 mg, 2.83 mmol) and triphenylphosphine (805 mg, 3.07 mmol) were added to an oven-dried 40 mL vial with a sitrbar and the vial was sealed with a septa-top vial and purged with vac/N₂. THF (7.9 mL) was added and the reaction was cooled to 0 C. DEAD (448 μl, 2.83 mmol) was added dropwise and the reaction mixture was allowed to warm to room temperature and was then stirred overnight. The reaction mixture was concentrated in vacuo and purified by silica gel chromatography (ISCO, 0-40% EtOAc/Hept) to give 2-fluoro-5-((4-(4,4,5,5-tetramethyl-1,3,2-dioxaborolan-2-yl)phenoxy)methyl)pyridine. LCMS Rt=1.04 min, m/z=330.3 [M+H]⁺.

Step 2: To an oven-dried 4 mL vial with a stirbar was added 2-fluoro-5-((4-(4,4,5,5-tetramethyl-1,3,2-dioxaborolan-2-yl)phenoxy)methyl)pyridine (39 mg, 0.08 mmol). The vial was sealed with a septa top cap and purged with vac/N₂ (×3). DMSO (0.45 mL) and cyclopentanamine (0.16 mL, 1.66 mmol) were added and the reaction mixture was heated to 80° C. and stirred overnight. The reaction mixture was diluted with EA, washed with water and brine, dried over Na₂SO₄, filtered and concentrated in vacuo. The residue was purified by silica gel chromatography (ISCO, 0-50% EA/heptane) to give N-cyclopentyl-5-((4-(4,4,5,5-tetramethyl-1,3,2-dioxaborolan-2-yl)phenoxy)methyl)pyridin-2-amine. LCMS t=1.03, m/z=395.3 [M+H].

Step 3: To a 4 mL vial with stirbar was added N-cyclopentyl-5-((4-(4,4,5,5-tetramethyl-1,3,2-dioxaborolan-2-yl)phenoxy)methyl)pyridin-2-amine (13 mg, 0.033 mmol, 5-bromo-2-oxo-6-(trifluoromethyl)-1,2-dihydropyridine-3-carboxamide (11.28 mg, 0.040 mmol, Tripotassium phosphate (20.99 mg, 0.099 mmol) and (dtbpf)PdCl₂ (2.149 mg, 3.30 μmol) and the reaction vial sealed with a septa top cap and purged with vac/N₂ (×3). Dioxane (247 μl) and water (82 μl) were added and the reaction mixture was heated to 65° C. and stirred overnight. The reaction mixture was diluted with EtOAc, filtered and concentrated in vacuo. The crude product was purified by prep-HPLC to give 5-(4-((6-(cyclopentylamino)pyridin-3-yl)methoxy)phenyl)-2-oxo-6-(trifluoromethyl)-1,2-dihydropyridine-3-carboxamide (167). ¹H NMR (500 MHz, DMSO-d6) δ 13.65 (br. s, 1H), 8.43 (br. s, 1H), 8.26 (s, 1H), 8.16 (br. s, 1H), 8.06 (d, J=2.1 Hz, 1H), 7.87 (d, J=9.3 Hz, 1H), 7.30 (d, J=8.7 Hz, 2H), 7.10 (d, J=8.7 Hz, 2H), 6.93 (br. s, 1H), 5.03 (s, 2H), 4.04 (dt, J=10.8, 6.2 Hz, 1H), 1.99 (dq, J=12.9, 6.6, 6.0 Hz, 2H), 1.74-1.65 (m, 2H), 1.63-1.47 (m, 4H). LCMS: Rt=0.72 min, m/z=473.3 [M+H].

### Example 168: 5-(4-((6-((cyclopropylmethyl)amino)pyridin-3-yl)methoxy)phenyl)-2-oxo-6-(trifluoromethyl)-1,2-dihydropyridine-3-carboxamide (168)

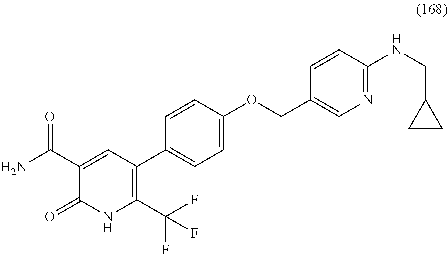 [SMILES: NC(=O)c1cc(-c2ccc(OCc3ccc(NCC4CC4)nc3)cc2)c(C(F)(F)F)[nH]c1=O]

5-(4-((6-((cyclopropylmethyl)amino)pyridin-3-yl)methoxy)phenyl)-2-oxo-6-(trifluoromethyl)-1,2-dihydropyridine-3-carboxamide (168) was made using a procedure similar to that used to make 5-(4-((6-(cyclopentylamino)pyridin-3-yl)methoxy)phenyl)-2-oxo-6-(trifluoromethyl)-1,2-dihydropyridine-3-carboxamide (167), except cyclopentanamine was replaced with cyclopropylmethanamine. ¹H NMR (500 MHz, DMSO-d6) δ 13.61 (br. s, 1H), 8.44 (br. s, 1H), 8.26 (s, 1H), 8.17 (br. s, 1H), 8.06 (d, J=2.2 Hz, 1H), 7.87 (br. d, J=9.1 Hz, 1H), 7.34-7.21 (m, 2H), 7.18-7.04 (m, 2H), 6.97 (br. s, 1H), 5.02 (s, 2H), 3.19 (dd, J=7.1, 2.9 Hz, 2H), 1.16-1.05 (m, 1H), 0.64-0.39 (m, 2H), 0.28 (dt, J=6.1, 4.3 Hz, 2H). LCMS: Rt=0.67 min, m/z=459.3 [M+H].

### Example 169: 5-(4-((6-(3,3-difluoroazetidin-1-yl)pyridin-3-yl)methoxy)phenyl)-2-oxo-6-(trifluoromethyl)-1,2-dihydropyridine-3-carboxamide (169)

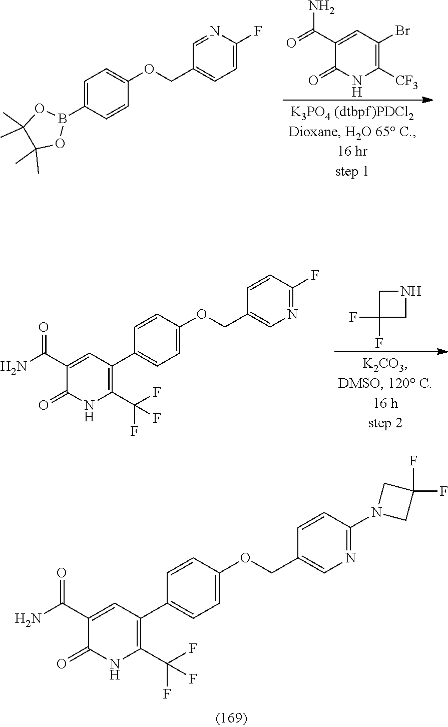

Step 1: 5-(4-((6-fluoropyridin-3-yl)methoxy)phenyl)-2-oxo-6-(trifluoromethyl)-1,2-dihydropyridine-3-carboxamide was obtained using the procedure described in step 3 of Example 167, except N-cyclopentyl-5-((4-(4,4,5,5-tetramethyl-1,3,2-dioxaborolan-2-yl)phenoxy)methyl)pyridin-2-amine was replaced with 2-fluoro-5-((4-(4,4,5,5-tetramethyl-1,3,2-dioxaborolan-2-yl)phenoxy)methyl)pyridine. LCMS: Rt=0.93 min, m/z=408.1 [M+H].

Step 2: To an oven dried 4 mL vial was added 5-(4-((6-fluoropyridin-3-yl)methoxy)phenyl)-2-oxo-6-(trifluoromethyl)-1,2-dihydropyridine-3-carboxamide (20 mg, 0.029 mmol), 3,3-difluoroazetidine hydrochloride (7.63 mg, 0.059 mmol) and potassium carbonate (16.29 mg, 0.118 mmol) and the vial was sealed with a septa-top cpa and purged with vac/N₂ (×3). DMSO (295 μl) was added and the reaction mixture was heated to 120° C. and stirred overnight. The reaction mixture was diluted with DCM, filtered and concentrated. The crude product was purified by prep-HPLC to give 5-(4-((6-(3,3-difluoroazetidin-1-yl)pyridin-3-yl)methoxy)phenyl)-2-oxo-6-(trifluoromethyl)-1,2-dihydropyridine-3-carboxamide (169). ¹H NMR (500 MHz, DMSO-d6) δ 13.63 (br. s, 1H), 8.45 (br. s, 1H), 8.29-8.25 (m, 2H), 8.17 (br. s, 1H), 7.76 (dd, J=8.5, 2.3 Hz, 1H), 7.30-7.23 (m, 2H), 7.13-7.04 (m, 2H), 6.64 (d, J=8.5 Hz, 1H), 5.05 (s, 2H), 4.40 (t, J=12.5 Hz, 4H); LCMS: Rt=0.81 min, m/z=481.1 [M+H].

### Example 170: 5-(4-((6-(3-methoxyazetidin-1-yl)pyridin-3-yl)methoxy)phenyl)-2-oxo-6-(trifluoromethyl)-1,2-dihydropyridine-3-carboxamide (170)

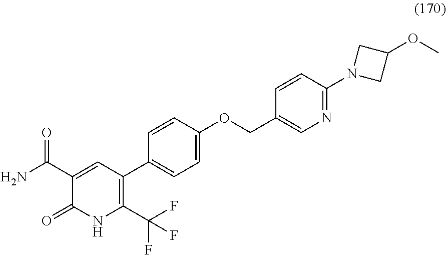 [SMILES: COC1CN(c2ccc(COc3ccc(-c4cc(C(N)=O)c(=O)[nH]c4C(F)(F)F)cc3)cn2)C1]

The title compound was prepared according to the procedure for example 167 was made using a procedure similar to that used to make 5-(4-((6-(3,3-difluoroazetidin-1-yl)pyridin-3-yl)methoxy)phenyl)-2-oxo-6-(trifluoromethyl)-1,2-dihydropyridine-3-carboxamide (169), except difluoroazetidine hydrochloride was replaced with 3-methoxyazetidine hydrochloride. ¹H NMR (500 MHz, DMSO-d6) δ 13.68 (br. s, 1H), 8.44 (br. s, 1H), 8.26 (s, 1H), 8.19-8.14 (m, 2H), 7.84 (br. s, 1H), 7.36-7.21 (m, 2H), 7.12-7.05 (m, 2H), 6.66 (s, 1H), 5.03 (s, 2H), 4.38-4.32 (m, 1H), 4.28 (t, J=8.5 Hz, 2H), 3.90 (d, J=8.5 Hz, 2H), 3.26 (s, 3H); LCMS: Rt=0.72 min, m/z=475.1 [M+H].

### Example 171: 5-(4-((2-cyclopropylpyrimidin-5-yl)methoxy)phenyl)-2-oxo-6-(trifluoromethyl)-1,2-dihydropyridine-3-carboxamide (171)

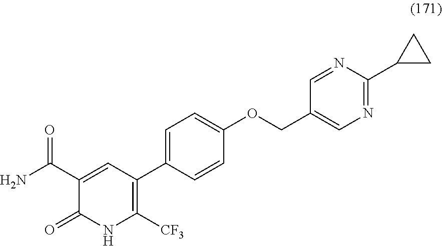 [SMILES: NC(=O)c1cc(-c2ccc(OCc3cnc(C4CC4)nc3)cc2)c(C(F)(F)F)[nH]c1=O]

5-(4-((2-cyclopropylpyrimidin-5-yl)methoxy)phenyl)-2-oxo-6-(trifluoromethyl)-1,2-dihydropyridine-3-carboxamide (171) was obtained using the procedures of step 1 and step 3 in Example 167, except (6-fluoropyridin-3-yl)methanol was replaced with (2-cyclopropylpyrimidin-5-yl)methanol, and N-cyclopentyl-5-((4-(4,4,5,5-tetramethyl-1,3,2-dioxaborolan-2-yl)phenoxy)methyl)pyridin-2-amine was replaced with 2-cyclopropyl-5-((4-(4,4,5,5-tetramethyl-1,3,2-dioxaborolan-2-yl)phenoxy)methyl)pyrimidine. LCMS: Rt=0.80 min, m/z=431.1 [M+H].

Note (2-cyclopropylpyrimidin-5-yl)methanol was obtained u using the following procedure:

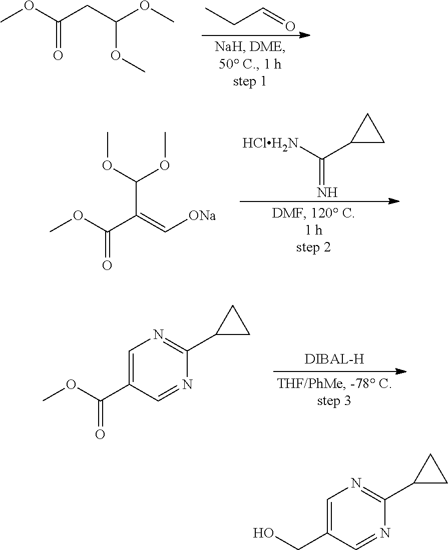

Step 1: To a 50 mL 2 necked flask with a stirbar, oven dried and cooled under argon, was added methyl 3,3-dimethoxypropanoate (0.71 mL, 5.0 mmol), methyl formate (0.74 mL, 12.0 mmol) and DME (10.0 mL). Sodium hydride (260 mg, 6.50 mmol) was added as a single portion and the reaction heated to 50° C. for 1 h. The reaction mixture was then cooled to rt and stirred overnight. The resulting suspension was diluted with Et₂O, filtered giving and dried under vacuum to give sodium 2-(dimethoxymethyl)-3-methoxy-3-oxoprop-1-en-1-olate which was used without further purification. LCMS Rt=0.28 min, no ionization.

Step 2: To an oven dried 4 mL vial with a stirbar was added sodium 2-(dimethoxymethyl)-3-methoxy-3-oxoprop-1-en-1-olate (238 mg, 1.20 mmol) and cyclopropanecarboximidamide hydrochloride (121 mg, 1.00 mmol). The reaction vial was sealed with a septa-top cap and purged with vac/N₂ (×3). DMF (1.0 mL) was added and the reaction was heated to 120° C. for 1 h. The reaction was diluted with DCM and filtered through a plug of Celite®. Following concentration in vacuo to give methyl 2-cyclopropylpyrimidine-5-carboxylate which was used in the without further purification. LCMS Rt=0.57 min, m/z=179.4 [M+H].

Step 3: To an oven dried 20 mL vial with a stirbar was added methyl 2-cyclopropylpyrimidine-5-carboxylate (166 mg, 0.93 mmol), and the reaction vial sealed with a septa-top cap and purged with vac/N₂ via needle (×3). THF (4.7 mL) was added and the reaction was cooled to −78° C. DIBAL-H (1.0 M in PhMe, 2.05 ml, 2.05 mmol) was then added dropwise and then the reaction mixture was allowed to slowly warm to rt in a cooling bath overnight. The reaction was quenched by addition of an aqueous solution of Rochelle's salt, diluted with EA and stirred vigorously for 30 mins. The layers were separated and the aqueous layer was extracted into EA. The combined organic layers were washed with brine, dried over Na₂SO₄, filtered and concentrated in vacuo. Purification by silica gel chromatography (ISCO, 50-100% EA/heptane) gave (2-cyclopropylpyrimidin-5-yl)methanol. LCMS Rt=0.29 min, m/z=151.1 [M+H].

### Example 172: 5-(4-((2-(cyclopentylmethyl)pyrimidin-5-yl)methoxy)phenyl)-2-oxo-6-(trifluoromethyl)-1,2-dihydropyridine-3-carboxamide (172)

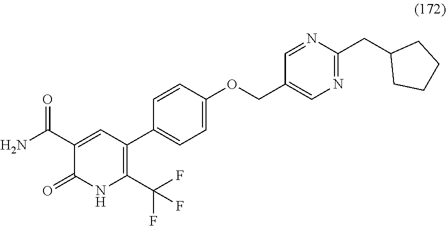 [SMILES: NC(=O)c1cc(-c2ccc(OCc3cnc(CC4CCCC4)nc3)cc2)c(C(F)(F)F)[nH]c1=O]

5-(4-((2-(cyclopentylmethyl)pyrimidin-5-yl)methoxy)phenyl)-2-oxo-6-(trifluoromethyl)-1,2-dihydropyridine-3-carboxamide (172) was obtained using the procedures of step 1 and step 3 in Example 167, except (6-fluoropyridin-3-yl)methanol was replaced with (2-(cyclopentylmethyl)pyrimidin-5-yl)methanol, and N-cyclopentyl-5-((4-(4,4,5,5-tetramethyl-1,3,2-dioxaborolan-2-yl)phenoxy)methyl)pyridin-2-amine was replaced with 2-(cyclopentylmethyl)-5-((4-(4,4,5,5-tetramethyl-1,3,2-dioxaborolan-2-yl)phenoxy)methyl)pyrimidine. LCMS: Rt=0.90 min, m/z=473.3 [M+H].

### Example 173: 5-(4-((2-neopentylpyrimidin-5-yl)methoxy)phenyl)-2-oxo-6-(trifluoromethyl)-1,2-dihydropyridine-3-carboxamide (173)

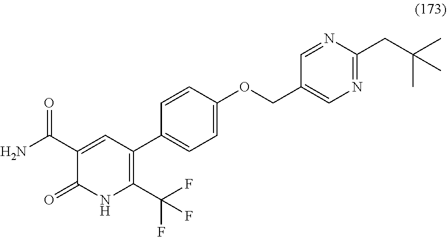 [SMILES: CC(C)(C)Cc1ncc(COc2ccc(-c3cc(C(N)=O)c(=O)[nH]c3C(F)(F)F)cc2)cn1]

5-(4-((2-neopentylpyrimidin-5-yl)methoxy)phenyl)-2-oxo-6-(trifluoromethyl)-1,2-dihydropyridine-3-carboxamide (173) was obtained using the procedures of step 1 and step 3 in Example 167, except (6-fluoropyridin-3-yl)methanol was replaced with ((2-neopentylpyrimidin-5-yl)methanol, and N-cyclopentyl-5-((4-(4,4,5,5-tetramethyl-1,3,2-dioxaborolan-2-yl)phenoxy)methyl)pyridin-2-amine was replaced with 2-neopentyl-5-((4-(4,4,5,5-tetramethyl-1,3,2-dioxaborolan-2-yl)phenoxy)methyl)pyrimidine. ¹H NMR (500 MHz, DMSO-d6) δ 13.63 (br, s, 1H), 8.86 (s, 2H), 8.55 (br. s, 1H), 8.25 (s, 2H), 8.11 (br. s, 1H), 7.30 (d, J=8.6 Hz, 2H), 7.18-7.09 (m, 2H), 5.19 (s, 2H), 2.79 (s, 2H), 0.96 (s, 9H). LCMS: Rt=0.90 min, m/z=473.3 [M+H].

### Example 174: 5-(4-((4-(cyclopentylamino)pyrimidin-5-yl)methoxy)phenyl)-2-oxo-6-(trifluoromethyl)-1,2-dihydropyridine-3-carboxamide (174)

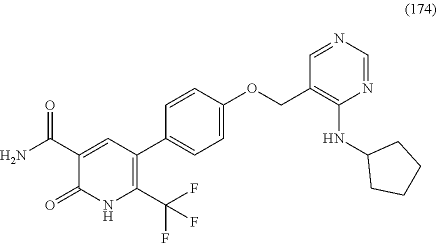 [SMILES: NC(=O)c1cc(-c2ccc(OCc3cncnc3NC3CCCC3)cc2)c(C(F)(F)F)[nH]c1=O]

5-(4-((4-(cyclopentylamino)pyrimidin-5-yl)methoxy)phenyl)-2-oxo-6-(trifluoromethyl)-1,2-dihydropyridine-3-carboxamide (174). ¹H NMR (400 MHz, DMSO-d6) δ 10.55 (s, 1H), 8.44 (s, 1H), 8.31 (s, 1H), 8.17 (s, 1H), 7.84 (s, 1H), 7.17 (d, J=8.6 Hz, 2H), 7.09 (s, 1H), 7.02 (d, J=8.8 Hz, 2H), 6.77 (d, J=7.2 Hz, 1H), 5.00 (s, 2H), 4.44 (d, J=7.0 Hz, 1H), 1.95 (d, J=6.2 Hz, 2H), 1.70 (s, 2H), 1.54 (s, 4H); LCMS: Rt=0.64 min, m/z=474.1 [M+H].

N-cyclopentyl-5-((4-(4,4,5,5-tetramethyl-1,3,2-dioxaborolan-2-yl)phenoxy)methyl)pyrimidin-4-amine, was obtained using the procedures of step 1 and step 3 in Example 167:

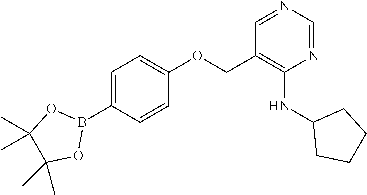 [SMILES: CC1(C)OB(c2ccc(OCc3cncnc3NC3CCCC3)cc2)OC1(C)C]

LCMS: Rt 0.821 min, m/z 396.1 [M+H].

### (4-(cyclopentylamino)pyrimidin-5-yl)methanol was Obtained Using the Following Procedure

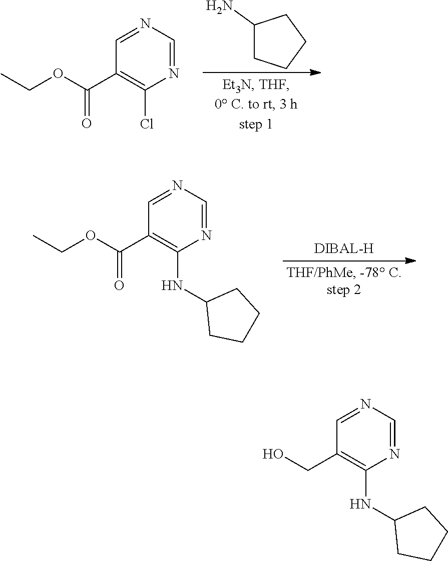 [SMILES: CCOC(=O)c1cncnc1Cl ; NC1CCCC1 ; CCOC(=O)c1cncnc1NC1CCCC1 ; OCc1cncnc1NC1CCCC1]

Step 1: To a solution of cyclopentanamine (456 mg, 5.36 mmol) in THF (10 mL) was added ethyl 4-chloropyrimidine-5-carboxylate (1.0 g, 5.36 mmol) at 0° C. followed by Et₃N (1.08 g, 10.7 mmol. The mixture was then stirred at 25° C. for 3 hr. The reaction mixture was poured into water (10 ml) and then extracted with EA (10 mL×2). The combined organic layers were dried over anhydrous Na₂SO₄, filtered and concentrated to give crude product which was purified by flash column chromatography to give ethyl 4-(cyclopentylamino)pyrimidine-5-carboxylate. TLC: PE/EA=3/1, Rf=0.2; ¹H NMR (400 MHz, DMSO-d6) δ 8.66 (d, J=7.0 Hz, 1H), 8.14 (d, J=6.8 Hz, 1H), 4.49-4.36 (m, 1H), 4.30 (q, J=7.2 Hz, 2H), 2.05-1.94 (m, 2H), 1.75-1.55 (m, 4H), 1.49 (td, J=6.1, 12.1 Hz, 2H), 1.31 (t, J=7.1 Hz, 3H).

Step 2: (4-(cyclopentylamino)pyrimidin-5-yl)methanol was obtained using the method of step 3 in Example 171. LCMS: Rt 0.544 min, m/z 194.1 [M+H].

### Example 175: 5-(4-((3-(cyclopropylmethyl)-3H-imidazo[4,5-b]pyridin-6-yl)methoxy)phenyl)-2-oxo-6-(trifluoromethyl)-1,2-dihydropyridine-3-carboxamide (175)

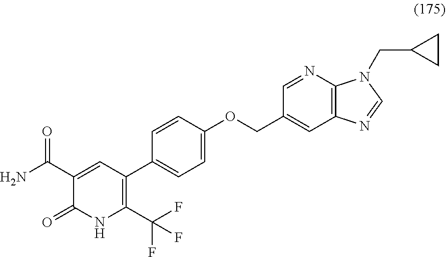 [SMILES: NC(=O)c1cc(-c2ccc(OCc3cnc4c(c3)ncn4CC3CC3)cc2)c(C(F)(F)F)[nH]c1=O]

5-(4-((3-(cyclopropylmethyl)-3H-imidazo[4,5-b]pyridin-6-yl)methoxy)phenyl)-2-oxo-6-(trifluoromethyl)-1,2-dihydropyridine-3-carboxamide (175) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 6-bromo-3-(cyclopropylmethyl)-3H-imidazo[4,5-b]pyridine. ¹H NMR (400 MHz, DMSO-d6) δ 10.50 (br. s, 1H), 8.56 (s, 1H), 8.52 (d, J=1.8 Hz, 1H), 8.29 (s, 1H), 8.20 (d, J=1.8 Hz, 1H), 7.85 (d, J=9.6 Hz, 1H), 7.17 (d, J=7.6 Hz, 2H), 7.06 (d, J=7.6 Hz, 2H), 5.27 (s, 2H), 4.17 (d, J=7.4 Hz, 2H), 1.37 (m, 1H), 0.54 (m, 2H), 0.48 (m, 2H); LCMS: Rt=0.67 min, m/z=484.1 [M+H].

### 6-bromo-3-(cyclopropylmethyl)-3H-imidazo[4,5-b]pyridine was Obtained Using the Following Procedure

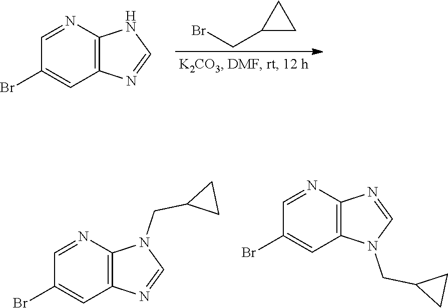 [SMILES: Brc1cnc2[nH]cnc2c1 ; BrCC1CC1 ; Brc1cnc2c(c1)ncn2CC1CC1 ; Brc1cnc2ncn(CC3CC3)c2c1]

To a solution of 6-bromo-3H-imidazo[4,5-b]pyridine (4.6 g, 23.2 mmol) and K₂CO₃ (4.8 g, 34.9 mmol) in DMF (50 mL) was added (bromomethyl)cyclopropane (4.7 g, 34.9 mmol) and the reaction was stirred at 25° C. for 12 hrs. The mixture was washed with H₂O (200 mL) and extracted with EA (100 mL×2). The combined organic layers were dried over anhydrous Na₂SO₄, filtered and concentrated. The residue was purified by prep-HPLC (formic acid) to give 6-bromo-3-(cyclopropylmethyl)-3H-imidazo[4,5-b]pyridine. LCMS: Rt=0.65 min, m/z=251.9, 253.9 [M+H]. ¹H NMR (400 MHz, DMSO) δ 8.59 (s, 1H), 8.45 (d, J=2.0 Hz, 1H), 8.37 (d, J=2.0 Hz, 1H), 8.13 (d, J=7.2 Hz, 1H), 1.36 (m, 1H), 0.52 (m, 2H), 0.48 (m, 2H).

### Methyl 3-(cyclopropylmethyl)-3H-imidazo[4,5-b]pyridine-6-carboxylate was Obtained Using the Following Procedure

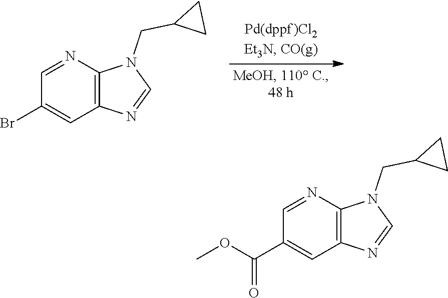 [SMILES: Brc1cnc2c(c1)ncn2CC1CC1 ; COC(=O)c1cnc2c(c1)ncn2CC1CC1]

To a solution of 6-bromo-3-(cyclopropylmethyl)-3H-imidazo[4,5-b]pyridine (2.2 g, 10.7 mmol) in MeOH (30 mL) was added Et₃N (2.65 g, 26.2 mmol), Pd(dppf)Cl₂ (319 mg, 0.44 mmol). The mixture was stirred under CO (2.5 MPa) at 110° C. for 48 h. The mixture was concentrated and the residue was purified by column (PE/EA=100/1) to give methyl 3-(cyclopropylmethyl)-3H-imidazo[4,5-b]pyridine-6-carboxylate. ¹H NMR (400 MHz, CDCl₃) δ 9.05 (s, 1H), 8.67 (d, J=1.8 Hz, 1H), 8.25 (s, 1H), 4.17 (d, J=7.2 Hz, 2H), 3.96 (s, 3H), 1.38 (m, 1H), 0.70 (m, 2H), 0.48 (m, 2H).

### Example 176: 5-(4-((6-chloropyridin-3-yl)methoxy)phenyl)-2-oxo-6-(trifluoromethyl)-1,2-dihydropyridine-3-carboxamide (176)

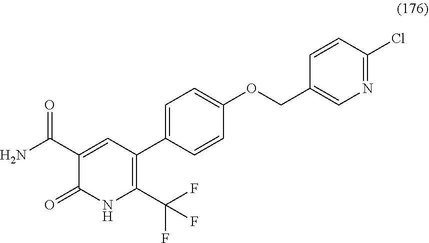 [SMILES: NC(=O)c1cc(-c2ccc(OCc3ccc(Cl)nc3)cc2)c(C(F)(F)F)[nH]c1=O]

5-(4-((6-chloropyridin-3-yl)methoxy)phenyl)-2-oxo-6-(trifluoromethyl)-1,2-dihydropyridine-3-carboxamide (176) was obtained using the procedures of step 1 and step 3 in Example 167, except (6-fluoropyridin-3-yl)methanol was replaced with (6-chloropyridin-3-yl)methanol, and N-cyclopentyl-5-((4-(4,4,5,5-tetramethyl-1,3,2-dioxaborolan-2-yl)phenoxy)methyl)pyridin-2-amine was replaced with 2-chloro-5-((4-(4,4,5,5-tetramethyl-1,3,2-dioxaborolan-2-yl)phenoxy)methyl)pyridine. ¹H NMR (500 MHz, Methanol-d4) δ 8.50 (d, J=2.3 Hz, 1H), 8.33 (s, 1H), 7.96 (dd, J=8.2, 2.3 Hz, 1H), 7.52 (d, J=8.2 Hz, 1H), 7.30 (d, J=8.6 Hz, 2H), 7.12 (d, J=8.6 Hz, 2H), 5.21 (s, 2H). LCMS Rt=0.91 min, m/z=424.1 [M+H].

### Example 177: 2-oxo-5-(4-((pyridin-3-yloxy)methyl)phenyl)-6-(trifluoromethyl)-1,2-dihydropyridine-3-carboxamide (177)

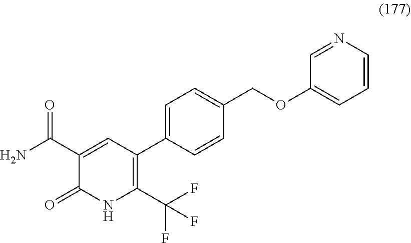 [SMILES: NC(=O)c1cc(-c2ccc(COc3cccnc3)cc2)c(C(F)(F)F)[nH]c1=O]

2-oxo-5-(4-((pyridin-3-yloxy)methyl)phenyl)-6-(trifluoromethyl)-1,2-dihydropyridine-3-carboxamide (177) was made using step 2 in the procedure used to make 5-(4-((3-nitrobenzyl)oxy)phenyl)-2-oxo-6-(trifluoromethyl-1,2-dihydropyridine-3-carboxamide (54), except 4,4,5,5-tetramethyl-2-(4-((3-nitrobenzyl)oxy)phenyl)-1,3,2-dioxaborolane was replaced with 3-((4-(4,4,5,5-tetramethyl-1,3,2-dioxaborolan-2-yl)benzyl)oxy)pyridine. ¹H NMR (400 MHz, DMSO-d6)=10.45 (br, s, 1H), 8.42 (s, 1H), 8.19 (m, 1H), 8.18 (m, 1H), 7.48 (m, 3H), 7.29 (m, 1H), 7.27 (m, 2H), 7.18 (m, 2H), 5.21 (s, 2H). LCMS: Rt=0.83 min, m/z=4390.1 [M+H].

3-((4-(4,4,5,5-tetramethyl-1,3,2-dioxaborolan-2-yl)benzyl)oxy)pyridine was obtained using the method described in step 2 of Example 52, except 4-(4-bromobenzylidene)tetrahydro-2H-pyran was replaced with 3-((4-bromobenzyl)oxy)pyridine.

### 3-((4-bromobenzyl)oxy)pyridine was Obtained Using the Following Procedure

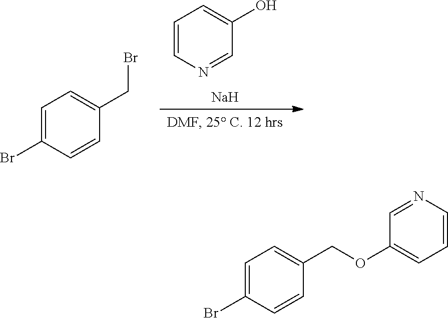

To a solution of 1-bromo-4-(bromomethyl)benzene (2.0 g, 8.00 mmol) and pyridin-3-ol (799 mg, 8.40 mmol) in DMF (5 mL) was added NaH (336 mg, 8.40 mmol) at 25° C. Then the mixture was stirred at 25° C. for 12 hrs. The reaction mixture was poured into water (20 ml) and then extracted with EA (20 mL×3). The combine organic layers were washed with saturated brine (30 mL×3), then dried over anhydrous Na₂SO₄, filtered and concentrated to give 3-((4-bromobenzyl)oxy)pyridine. LCMS: Rt=1.84 min, m/z=365.1 [M+H].

### Example 178: 5-(4-((5-(furan-3-yl)pyridin-3-yl)methoxy)phenyl)-2-oxo-6-(trifluoromethyl)-1,2-dihydropyridine-3-carboxamide (178)

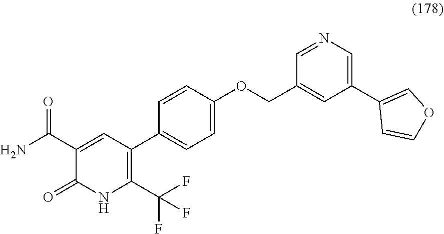 [SMILES: NC(=O)c1cc(-c2ccc(OCc3cncc(-c4ccoc4)c3)cc2)c(C(F)(F)F)[nH]c1=O]

5-(4-((5-(furan-3-yl)pyridin-3-yl)methoxy)phenyl)-2-oxo-6-(trifluoromethyl)-1,2-dihydropyridine-3-carboxamide (178) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-(furan-3-yl)pyridin-3-yl)methanol. ¹H NMR (400 MHz, DMSO-d6) δ 8.87 (s, 1H), 8.58 (s, 1H), 8.36 (s, 1H), 8.14 (s, 1H), 7.96 (s, 1H), 7.81 (s, 1H), 7.21 (d, J=8.0 Hz, 2H), 7.11-7.05 (m, 3H), 5.19 (s, 2H); LCMS: Rt—0.91 min, m/z=456.1 [M+H].

### (5-(furan-3-yl)pyridin-3-yl)methanol was Obtained Using the Following Procedure

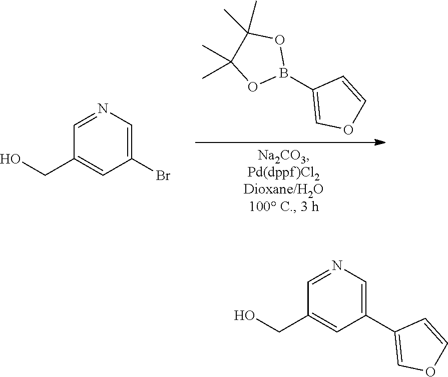

To a solution of (5-bromopyridin-3-yl)methanol (1.0 g, 5.32 mmol), 2-(furan-3-yl)-4,4,5,5-tetramethyl-1,3,2-dioxaborolane (1.24 g, 6.38 mmol) and Na₂CO₃ (1.13 g, 10.6 mmol) in a mixture of dioxane/H₂O (5:1, 10 mL) was added Pd(dppf)Cl₂ (390 mg, 0.53 mmol) at 25° C. under N₂ and the mixture was stirred at 100° C. for 3 hr. The reaction mixture was poured into water (20 mL) and then extracted with EA (20 mL×2). The combined organic layers were dried over anhydrous Na₂SO₄, filtered and concentrated to give crude product which was purified by column chromatography (PE/EA=3/1 to PE/EA=1/1) to give (5-(furan-3-yl)pyridin-3-yl)methanol. LCMS: Rt=0.24 min, m/z=176.1 [M+H]; TLC: (PE/EA=1/1) Rt=0.2.

### Example 179: 5-(4-((5-morpholinopyridin-3-yl)methoxy)phenyl)-2-oxo-6-(trifluoromethyl)-1,2-dihydropyridine-3-carboxamide (179)

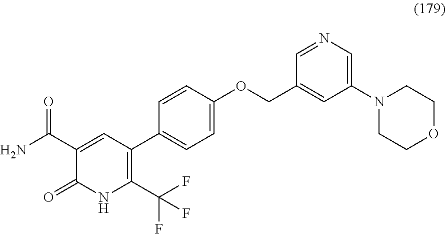 [SMILES: NC(=O)c1cc(-c2ccc(OCc3cncc(N4CCOCC4)c3)cc2)c(C(F)(F)F)[nH]c1=O]

5-(4-((5-morpholinopyridin-3-yl)methoxy)phenyl)-2-oxo-6-(trifluoromethyl-1,2-dihydropyridine-3-carboxamide (179) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(5-(bromomethyl)pyridin-3-yl)morpholine. ¹H NMR (400 MHz, DMSO-d6) δ 9.34 (s, 1H), 8.29 (d, J=2.6 Hz, 1H), 8.16 (s, 1H), 8.14 (s, 1H), 8.10 (s, 1H), 7.75 (s, 1H), 7.43 (s, 1H), 7.24 (d, J=8.4 Hz, 2H), 7.09 (d, J=8.4 Hz, 2H), 5.12 (s, 2H), 3.77 (m, 5H), 3.21 (m, 4H); LCMS: Rt=0.58 min, m/z=475.1 [M+H].

### Example 180: 2-oxo-5-(4-((5-(tetrahydrofuran-3-yl)pyridin-3-yl)methoxy)phenyl)-6-(trifluoromethyl)-1,2-dihydropyridine-3-carboxamide (180)

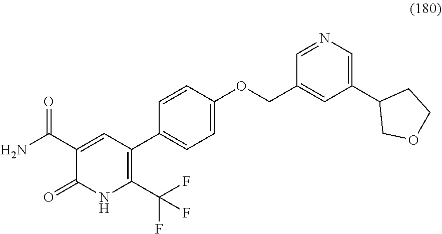 [SMILES: NC(=O)c1cc(-c2ccc(OCc3cncc(C4CCOC4)c3)cc2)c(C(F)(F)F)[nH]c1=O]

2-oxo-5-(4-((5-(tetrahydrofuran-3-yl)pyridin-3-yl)methoxy)phenyl)-6-(trifluoromethyl)-1,2-dihydropyridine-3-carboxamide (180) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-ylethan-1-one was replaced with (5-(tetrahydrofuran-3-yl)pyridin-3-yl)methanol. ¹H NMR (400 MHz, DMSO-d6) δ 8.56 (d, J=2.0 Hz, 1H), 8.50 (d, J=2.0 Hz, 1H), 7.97 (s, 1H), 7.82 (t, J=2.0 Hz, 1H), 7.20 (d, J=8.4 Hz, 2H), 7.10 (s, 1H), 7.06 (d, J=8.4 Hz, 2H), 5.17 (s, 2H), 4.09-4.03 (m, 1H), 4.01-3.93 (m, 1H), 3.82 (q, J=7.6 Hz, 1H), 3.63-3.57 (m, 1H), 3.51-3.42 (m, 1H), 2.41-2.34 (m, 1H), 2.00-1.90 (m, 1H); LCMS: Rt=0.82 min, m/z=460.1 [M+H].

### (5-(tetrahydrofuran-3-yl)pyridin-3-yl)methanol was Obtained Using the Following Procedure

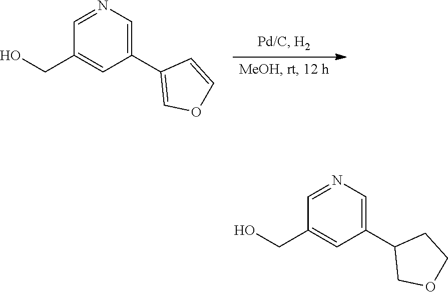 [SMILES: OCc1cncc(-c2ccoc2)c1 ; "MeOH, rt, 12 h" ; "Pd/C, H2" ; OCc1cncc(C2CCOC2)c1]

Pd/C (30 mg) was added to a solution of (5-(furan-3-yl)pyridin-3-yl)methanol (300 mg, 1.72 mmol) in MeOH (5 mL) at 25° C. and the mixture stirred at 25° C. for 12 hrs under H₂ (15 psi). The reaction mixture was filtered and concentrated to give (5-(tetrahydrofuran-3-yl)pyridin-3-yl)methanol which was used without further purification. LCMS: Rt=0.12 min, m/z=180.1 [M+H].

### Example 181: 5-(4-((5-(3-hydroxyoxetan-3-yl)pyridin-3-yl)methoxy)phenyl)-2-oxo-6-(trifluoromethyl)-1,2-dihydropyridine-3-carboxamide (181)

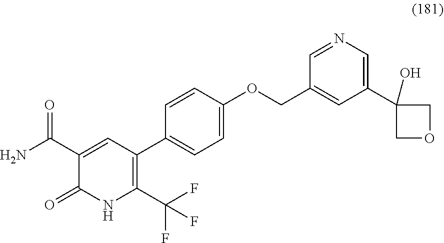 [SMILES: NC(=O)c1cc(-c2ccc(OCc3cncc(C4(O)COC4)c3)cc2)c(C(F)(F)F)[nH]c1=O]

5-(4-((5-(3-hydroxyoxetan-3-yl)pyridin-3-yl)methoxy)phenyl)-2-oxo-6-(trifluoromethyl)-1,2-dihydropyridine-3-carboxamide (181) was made using step 2 in the procedure used to make 5-(4-((3-nitrobenzyl)oxy)phenyl)-2-oxo-6-(trifluoromethyl)-1,2-dihydropyridine-3-carboxamide (54), except 4,4,5,5-tetramethyl-2-(4-((3-nitrobenzyl)oxy)phenyl)-1,3,2-dioxaborolane was replaced with 3-(5-((4-(4,4,5,5-tetramethyl-1,3,2-dioxaborolan-2-yl)phenoxy)methyl)pyridin-3-yl)oxetan-3-ol. ¹H NMR (400 MHz, DMSO-d6) δ 8.80 (d, J=4.8 Hz, 1H), 8.63 (d, J=11.2 Hz, 1H), 8.07 (d, J=11.6 Hz, 1H), 7.97 (s, 1H), 7.75 (d, J=8.6 Hz, 1H), 7.16 (d, J=8.6 Hz, 1H), 7.02 (d, J=16.2 Hz, 2H), 6.65 (s, 1H), 5.21 (s, 2H), 4.82-4.79 (m, 2H), 4.75-4.72 (m, 2H); LCMS: Rt=0.57 min, m/z=462.0 [M+H].

3-(5-((4-(4,4,5,5-tetramethyl-1,3,2-dioxaborolan-2-yl)phenoxy)methyl)pyridin-3-yl)oxetan-3-ol was obtained using the method described in step 2 of Example 52, except 4-(4-bromobenzylidene)tetrahydro-2H-pyran was replaced with 3-(5-((4-bromophenoxy)methyl)pyridin-3-yl)oxetan-3-ol.

### 3-(5-((4-bromophenoxy)methyl)pyridin-3-yl)oxetan-3-ol was Obtained Using the Following Procedure

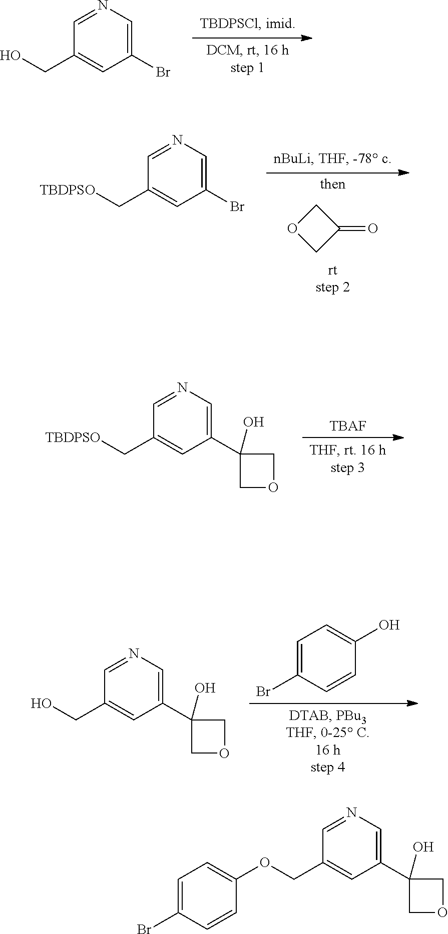

Step 1: TBDPSCl (17.5 g, 60 mmol) was added drop-wise to a solution of (5-bromopyridin-3-yl)methanol (10 g, 50 mmol) and imidazole (7.0 g, 100 mmol) in DCM (100 mL) and the mixture stirred at 25° C. for 16 hr. The reaction mixture was poured into water (200 mL) and the resulting mixture was extracted with EA (200 mL×2). The combined organic layers were dried over anhydrous Na₂SO₄, filtered and concentrated to give crude product which was purified by column chromatography (20-50% PE:EA) to give 3-bromo-5-(((tert-butyidiphenylsilyl)oxy)methyl)pyridine. TLC:PE:EA=1:1 Rf=0.8.

Step 2: nBuLi (20 mL, 50 mmol) was added drop-wise to a solution of 3-bromo-5-(((tert-butyldiphenylsilyl)oxy)methyl)pyridine (10 g, 25 mmol) in THF (100 mL) at −78° C. and the mixture stirred at −25° C. for 1 hr. Oxetan-3-one (3.5 mg, 50 mmol) was then added at 0° C. and the mixture stirred at 25° C. for 15 hr. The reaction mixture was poured into water (200 mL) and the resulting mixture extracted with EA (200 mL×2). The combined organic layers were dried over anhydrous Na₂SO₄, filtered and concentrated to give crude product which was purified by column chromatography (20-50% PE:EA) to give 3-(5-(((tert-butyldiphenylsilyl)oxy)methyl)pyridin-3-yl)oxetan-3-ol. TLC: (15% PE:EA) Rf=0.2; LCMS: Rt=0.84 min, m/z=412.0 [M+H].

Step 3: TBAF (1.3 g, 5.2 mmol) was added to a solution of 3-(5-(((tert-butyldiphenylsilyl)oxy)methyl)pyridin-3-yl)oxetan-3-ol (2.2 g, 5.2 mmol) in THF (20 mL) at 25° C. and the mixture stirred at 25° C. for 16 hr. The reaction mixture was concentrated to give crude product which was purified by prep-TLC (PE:EA=1:1) Rf=0.1 to give 3-(5-(hydroxymethyl)pyridin-3-yl)oxetan-3-ol. ¹H NMR (400 MHz, CDCl₃) δ 8.64 (d, J=2.0 Hz, 1H), 8.38 (d, J=1.8 Hz, 1H), 7.93 (s, 1H), 4.88 (d, J=7.2 Hz, 2H), 4.75 (d, J=7.2 Hz, 2H), 4.66 (s, 2H).

Step 4: DTBAD (800 mg, 4 mmol) was added to a solution of 3-(5-(hydroxymethyl)pyridin-3-yl)oxetan-3-ol (360 mg, 2.0 mmol), 4-bromophenol (440 mg, 2 mmol) and PBu3 (800 mg, 4 mmol) in THF (4 mL) at 0° C. and the mixture stirred at 25° C. for 16 hr. The reaction mixture was poured into water (20 mL) and extracted with EA (20 mL×2). The combined organic layers were dried over anhydrous Na₂SO₄, filtered and concentrated to give crude product which was purified by prep-TLC (1:1 PE/EA) to give 3-(5-((4-bromophenoxy)methyl)pyridin-3-yl)oxetan-3-ol. TLC: (1:1 PE:EA) Rf=0.5; LCMS: Rt=0.64 min, m/z=336.0 [M+H]; ¹H NMR (400 MHz, CDCl3) δ (d, J=2.0 Hz, 1H), 8.62 (d, J=2.0 Hz, 1H), 8.11 (s, 1H), 7.41 (d, J=9.0 Hz, 2H), 6.87 (d, J=9.0 Hz, 2H), 5.08 (s, 2H), 5.03 (d, J=7.2 Hz, 2H), 4.83 (d, J=7.2 Hz, 2H).

### Example 182: 5-(4-((5-(3-fluorooxetan-3-yl)pyridin-3-yl)methoxy)phenyl)-2-oxo-6-(trifluoromethyl)-1,2-dihydropyridine-3-carboxamide (182)

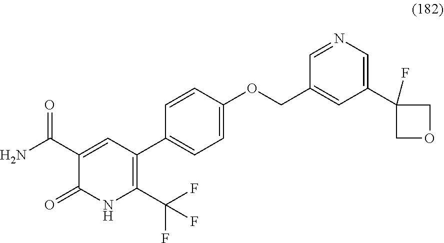 [SMILES: NC(=O)c1cc(-c2ccc(OCc3cncc(C4(F)COC4)c3)cc2)c(C(F)(F)F)[nH]c1=O]

5-(4-((5-(3-fluorooxetan-3-yl)pyridin-3-yl)methoxy)phenyl)-2-oxo-6-(trifluoromethyl)-1,2-dihydropyridine-3-carboxamide (182) was made using step 2 in the procedure used to make 5-(4-((3-nitrobenzyl)oxy)phenyl)-2-oxo-6-(trifluoromethyl)-1,2-dihydropyridine-3-carboxamide (54), except 4,4,5,5-tetramethyl-2-(4-((3-nitrobenzyl)oxy)phenyl)-1,3,2-dioxaborolane was replaced with 3-(3-fluorooxetan-3-yl)-5-((4-(4,4,5,5-tetramethyl-1,3,2-dioxaborolan-2-yl)phenoxy)methyl)pyridine. ¹H NMR (400 MHz, DMSO-d6) δ 8.79 (s, 1H), 8.77 (s, 1H), 8.11 (s, 1H), 8.11 (s, 1H), 7.96 (s, 1H), 7.29 (d, J=8.4 Hz, 2H), 7.14 (d, J=8.8 Hz, 2H), 5.27 (s, 2H), 5.04 (s, 1H), 4.98 (s, 2H); LCMS: Rt=0.64 min, m/z=464.1 [M+H].

3-(3-fluorooxetan-3-yl)-5-((4-(4,4,5,5-tetramethyl-1,3,2-dioxaborolan-2-yl)phenoxy)methyl)pyridine was obtained using the method described in step 2 of Example 52, except 4-(4-bromobenzylidene)tetrahydro-2H-pyran was replaced with 3-((4-bromophenoxy)methyl)-5-(3-fluorooxetan-3-yl)pyridine.

### 3-((4-bromophenoxy)methyl)-5-(3-fluorooxetan-3-yl)pyridine was Obtained Using the Following Procedure

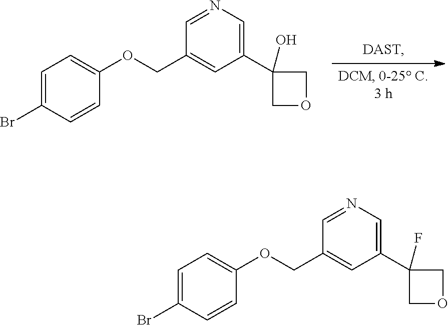 [SMILES: OC1(c2cncc(COc3ccc(Br)cc3)c2)COC1 ; "DAST," ; "DCM, 0-25° C.3 h" ; FC1(c2cncc(COc3ccc(Br)cc3)c2)COC1]

DAST (287 mg, 1.78 mmol) was added to a solution of 3-(5-((4-bromophenoxy)methyl)pyridin-3-yl)oxetan-3-ol (200 mg, 0.59 mmol) in DCM (5 mL) at 0° C. Then the mixture was stirred at 25° C. for 3 hrs. The reaction mixture was poured into water (10 mL) and saturated NaHCO₃ solution was added to adjust pH=7. The resulting mixture was extracted with DCM (10 mL×2). The combined organic layers were dried over anhydrous Na₂SO₄, filtered and concentrated to give crude product which was purified by column chromatography (PE/EA=5:1) to give 3-((4-bromophenoxy)methyl)-5-(3-fluorooxetan-3-yl)pyridine. TLC: (PE/EA=3:1) Rf=0.3; LCMS: Rt=0.77 min, m/z=338.0, 340.1 [M+1]+=338/340; ¹H NMR (400 MHz, DMSO-d6) δ 8.77-8.72 (m, 2H), 8.06 (s, 1H), 7.49 (d, J=9.2 H z, 2H), 7.05 (d, J=8.8 Hz, 2H), 5.21 (s, 2H), 5.02 (s, 2H), 4.97 (s, 2H).

### Example 183: 2-oxo-5-(4-((3-(pyridin-3-yl)oxetan-3-yl)oxy)phenyl)-6-(trifluoromethyl)-1,2-dihydropyridine-3-carboxamide (183)

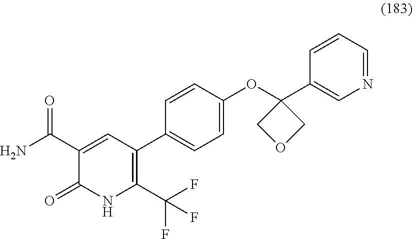 [SMILES: NC(=O)c1cc(-c2ccc(OC3(c4cccnc4)COC3)cc2)c(C(F)(F)F)[nH]c1=O]

2-oxo-5-(4-((3-(pyridin-3-yl)oxetan-3-yl)oxy)phenyl)-6-(trifluoromethyl)-1,2-dihydropyridine-3-carboxamide (183) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 3-(pyridin-3-yl)oxetan-3-ol. ¹H NMR (500 MHz, DMSO-d6) δ 13.69 (br. s, 1H), 8.82 (d, J=2.2 Hz, 1H), 8.59 (dd, J=4.9, 1.5 Hz, 1H), 8.45 (br. s, 1H), 8.23 (s, 1H), 8.18 (br. s, 1H), 8.04 (dt, J=7.9, 2.2 Hz, 1H), 7.52 (dd, J=8.0, 4.9 Hz, 1H), 7.20 (d, J=8.5 Hz, 2H), 6.70-6.63 (m, 2H), 5.07 (d, J=7.5 Hz, 1H), 5.03 (d, J=7.5 Hz, 1H). LCMS: Rt=0.54 min. m/z=432.3 [M+H].

### 3-(pyridin-3-yl)oxetan-3-ol was Obtained Using the Following Procedure

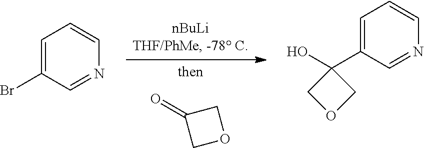

3-bromopyridine (0.06 mL, 0.6 mmol) in toluene (1.0 mL) was added to an oven dried 8 mL vial with a stirbar which was sealed with a septa top cap and purged with vac/N₂ (×3). The reaction was cooled to −78° C. and nBuLi (1.6 M in hexanes, 0.39 mL, 0.63 mmol) was added dropwise and the resulting solution was stirred at −78° C. for 30 min. A solution of oxetan-3-one (0.032 mL, 0.5 mmol) in THF (1.0 ml) was then added dropwise and the reaction mixture was allowed to warm to rt slowly overnight. The reaction was quenched with NH₄Cl_{(aq)}, and extracted with EA (×3). The combined organic layers were washed with brine, dried over Na₂SO₄, filtered and concentrated in vacuo to give 3-(pyridin-3-yl)oxetan-3-ol which was used without further purification. LCMS Rt=0.14 min, m/z=152.1 [M+H].

### Example 184: 5-(4-(cyclopropyl(pyridin-3-yl)methoxy)phenyl)-2-oxo-6-(trifluoromethyl)-1,2-dihydropyridine-3-carboxamide (184)

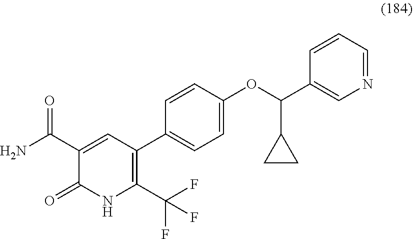 [SMILES: NC(=O)c1cc(-c2ccc(OC(c3cccnc3)C3CC3)cc2)c(C(F)(F)F)[nH]c1=O]

5-(4-(cyclopropyl(pyridin-3-yl)methoxy)phenyl)-2-oxo-6-(trifluoromethyl)-1,2-dihydropyridine-3-carboxamide (184) was obtained using the procedures of step 1 and step 3 in Example 167, except (6-fluoropyridin-3-yl)methanol was replaced with cyclopropyl(pyridin-3-yl)methanol, and N-cyclopentyl-5-((4-(4,4,5,5-tetramethyl-1,3,2-dioxaborolan-2-yl)phenoxy)methyl)pyridin-2-amine was replaced with 3-(cyclopropyl(4-(4,4,5,5-tetramethyl-1,3,2-dioxaborolan-2-yl)phenoxy)methyl)pyridine. LCMS Rt=0.64 min, m/z=430.3 [M+H].

### Cyclopropyl(pyridin-3-yl)methanol was Obtained Using the Following Procedure

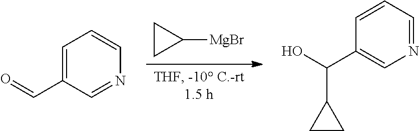

Nicotinaldehyde (94 μl, 1.0 mmol) and THF (4.0 mL) were added to an oven dried 20 mL vial with a stirbar which was sealed with a septa top cap and purged with vac/N₂ (×3). The reaction mixture was cooled to −10° C. and cyclopropylmagnesium bromide (1.0 M in THF, 1.30 mL, 1.30 mmol) was added dropwise. The reaction mixture was then allowed to slowly warm to rt in a cooling bath and then stirred at rt for 1.5 h. The reaction was quenched with NH₄Cl_{(aq)}, extracted with EA and the combined organic layers were washed with brine, dried over Na₂SO₄, filtered and concentrated. The crude product was then purified by silica gel chromatography (ISCO, 50-100% EA/heptane) to give cyclopropyl(pyridin-3-yl)methanol. LCMS Rt=0.23 min, m/z=150.1 [M+H].

### Example 185: 2-oxo-5-(4-((1-(pyridin-3-yl)pent-4-en-1-yl)oxy)phenyl)-6-(trifluoromethyl)-1,2-dihydropyridine-3-carboxamide (185)

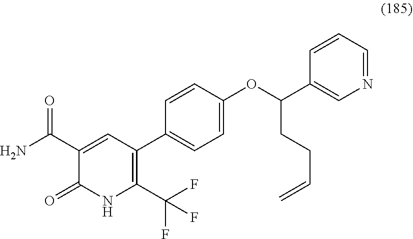 [SMILES: C=CCCC(Oc1ccc(-c2cc(C(N)=O)c(=O)[nH]c2C(F)(F)F)cc1)c1cccnc1]

2-oxo-5-(4-((1-(pyridin-3-yl)pent-4-en-1-yl)oxy)phenyl)-6-(trifluoromethyl)-1,2-dihydropyridine-3-carboxamide (185) was obtained using the procedures of step 1 and step 3 in Example 167, except (6-fluoropyridin-3-yl)methanol was replaced with 1-(pyridin-3-yl)pent-4-en-1-ol, and N-cyclopentyl-5-((4-(4,4,5,5-tetramethyl-1,3,2-dioxaborolan-2-yl)phenoxy)methyl)pyridin-2-amine was replaced with 3-(1-(4-(4,4,5,5-tetramethyl-1,3,2-dioxaborolan-2-yl)phenoxy)pent-4-en-1-yl)pyridine. ¹H NMR (500 MHz, DMSO-d6) δ 13.65 (br. s, 1H), 8.66 (d, J=2.2 Hz, 1H), 8.49 (dd, J=4.8, 1.7 Hz, 1H), 8.17 (s, 1H), 8.24-7.91 (br. S, 2H), 7.84 (dt, J=7.8, 2.2 Hz, 1H), 7.39 (ddd, J=7.8, 4.8, 0.8 Hz, 1H), 7.17 (d, J=8.3 Hz, 2H), 7.07-6.92 (m, 2H), 5.88 (ddt, J=16.9, 10.3, 6.4 Hz, 1H), 5.48 (dd, J=7.6, 5.2 Hz, 1H), 5.03 (dd, J=16.9, 1.8 Hz, 1H), 5.01-4.96 (m, 1H), 2.28-2.02 (m, 3H), 1.96-1.84 (m, 1H). LCMS Rt=0.83 min, m/z=444.0 [M+H].

### Example 186: 5-(4-(2-methoxy-1-(pyridin-3-yl)ethoxy)phenyl)-2-oxo-6-(trifluoromethyl)-1,2-dihydropyridine-3-carboxamide (186)

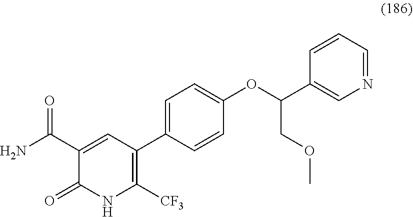 [SMILES: COCC(Oc1ccc(-c2cc(C(N)=O)c(=O)[nH]c2C(F)(F)F)cc1)c1cccnc1]

5-(4-(2-methoxy-1-(pyridin-3-yl)ethoxy)phenyl)-2-oxo-6-(trifluoromethyl)-1,2-dihydropyridine-3-carboxamide (186) was made using step 2 in the procedure used to make 5-(4-((3-nitrobenzyl)oxy)phenyl)-2-oxo-6-(trifluoromethyl)-1,2-dihydropyridine-3-carboxamide (54), except 4,4,5,5-tetramethyl-2-(4-((3-nitrobenzyl)oxy)phenyl)-1,3,2-dioxaborolane was replaced with 3-(2-methoxy-1-(4-(4,4,5,5-tetramethyl-1,3,2-dioxaborolan-2-yl)phenoxy)ethyl)pyridine. LCMS Rt=0.59 min, m/z=434.3 [M+H].

3-(2-methoxy-1-(4-(4,4,5,5-tetramethyl-1,3,2-dioxaborolan-2-yl)phenoxy)ethyl)pyridine was obtained using the method described in step 2 of Example 52, except 4-(4-bromobenzylidene)tetrahydro-2H-pyran was replaced with 3-(1-(4-bromophenoxy)-2-methoxyethyl)pyridine.

### 3-(1-(4-bromophenoxy)-2-methoxyethyl)pyridine was Obtained Using the Following Procedure

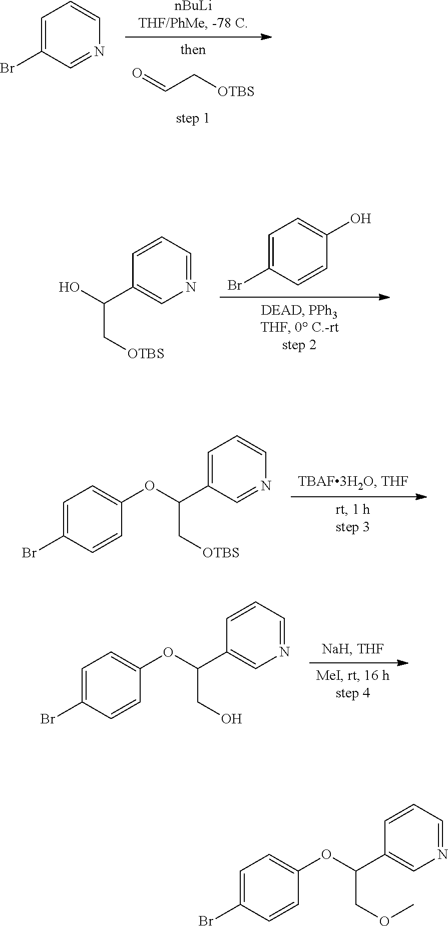

Step 1: To a 100 mL schlenk flask, dried under vacuum with a heat gun and purged with vac/N₂ (×3) was added 3-bromopyridine (1.16 mL, 12.0 mmol) and toluene (20 mL). The reaction was cooled to −78° C. and nBuLi (2.5 M in hexanes, 5.00 mL, 12.5 mmol) was added dropwise and the reaction mixture was stirred at −78° C. for 30 min. A solution of 2-((tert-butyldimethylsilyl)oxy)acetaldehyde (1.90 mL, 10.0 mmol) in THF (10.0 mL) was then added dropwise and the reaction mixture then allowed to warm to rt slowly overnight. The reaction was quenched with NH₄Cl_{(aq)}, extracted into EA×3, and the combined organic layers were washed with brine, dried over Na₂SO₄, filtered and concentrated in vacuo. The crude product was purified by silica gel chromatography (ISCO, 0-70% EA/heptane) to give 2-((tert-butyldimethylsilyl)oxy)-1-(pyridin-3-yl)ethan-1-ol. LCMS Rt=0.71 min, m/z=254.3 [M+H].

Step 2: 3-(1-(4-bromophenoxy)-2-((tert-butyldimethylsilyl)oxy)ethyl)pyridine was prepared using the method described in step 1 in Example 167, using 2-((tert-butyldimethylsilyl)oxy)-1-(pyridin-3-yl)ethan-1-ol and 4-bromophenol. LCMS t=1.12, m/z=408.2, 410.2 [M+H].

Step 3: To a 4 mL vial with a stirbar was added 3-(1-(4-bromophenoxy)-2-((tert-butyldimethylsilyl)oxy)ethyl)pyridine (30 mg, 0.07 mmol) and the vial was sealed with a septa top cap, purged with vac/N₂ (×3). THF (0.37 mL) was added, followed by TBAF (1.0 M in THF, 110 μl, 0.11 mmol) and the reaction mixture was stirred at rt for 1 h. The reaction mixture was diluted with EA, washed sequentially with water and brine, dried over Na₂SO₄, filtered through a plug of SiO₂ and concentrated in vacuo to give 2-(4-bromophenoxy)-2-(pyridin-3-yl)ethan-1-ol. LCMS t=0.70, m/z=294.1, 296.1 [M+H].

Step 4: To an oven dried 4 mL vial with a stirbar was added NaH (60% in mineral oil, 3.8 mg, 0.10 mmol) and the vial was sealed with a septa-top cap and purged with vac/N₂ (×3). A solution of 2-(4-bromophenoxy)-2-(pyridin-3-yl)ethan-1-ol (21.0 mg, 0.07 mmol) in THF (0.37 mL) was added slowly. The reaction mixture was stirred at rt for 30 mins and iodomethane (6.4 μl, 0.10 mmol) was added. The reaction was then stirred at rt overnight. The reaction mixture was diluted with EA, washed with brine and the organic layer dried over Na₂SO₄, filtered and concentrated to give 3-(1-(4-bromophenoxy)-2-methoxyethyl)pyridine. LCMS t=0.80, m/z=306.1, 308.1 [M+H].

### Example 187: 5-(4-(2-hydroxy-1-(pyrazin-2-yl)ethoxy)phenyl)-2-oxo-6-(trifluoromethyl)-1,2-dihydropyridine-3-carboxamide (187)

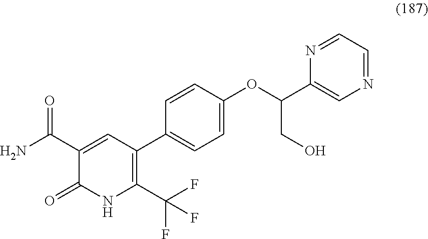 [SMILES: NC(=O)c1cc(-c2ccc(OC(CO)c3cnccn3)cc2)c(C(F)(F)F)[nH]c1=O]

5-(4-(2-hydroxy-1-(pyrazin-2-yl)ethoxy)phenyl)-2-oxo-6-(trifluoromethyl)-1,2-dihydropyridine-3-carboxamide (187) was made using the method described for the synthesis of 5-(4-(2-methoxy-1-(pyridin-3-yl)ethoxy)phenyl)-2-oxo-6-(trifluoromethyl)-1,2-dihydropyridine-3-carboxamide (186), except 3-(1-(4-(4,4,5,5-tetramethyl-1,3,2-dioxaborolan-2-yl)phenoxy)pent-4-en-1-yl)pyridine was replaced with 2-(pyrazin-2-yl)-2-(4-(4,4,5,5-tetramethyl-1,3,2-dioxaborolan-2-yl)phenoxy)ethan-1-ol. ¹H NMR (500 MHz, DMSO-d6) δ 13.66 (br. s, 1H), 8.80-7.80 (br. m, 2H). 8.74 (d, J=1.4 Hz, 1H), 8.67 (t, J=2.0 Hz, 1H), 8.59 (d, J=2.6 Hz, 1H), 8.18 (s, 1H), 7.20 (d, J=8.2 Hz, 2H), 7.02 (d, J=8.3 Hz, 2H), 5.49 (t, J=5.1 Hz, 1H), 5.22 (t, J=5.8 Hz, 1H), 3.91 (t, J=5.5 Hz, 2H). LCMS: Rt=0.64 min, m/z=421.1 [M+H].

### Example 188: 5-(4-((4-acetylmorpholin-2-yl)methoxy)phenyl)-2-oxo-6-(trifluoromethyl)-1,2-dihydropyridine-3-carboxamide (188)

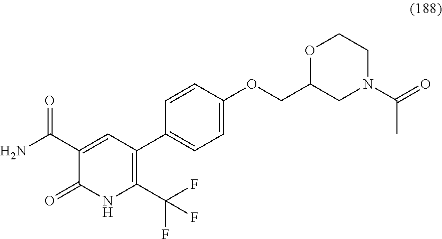 [SMILES: CC(=O)N1CCOC(COc2ccc(-c3cc(C(N)=O)c(=O)[nH]c3C(F)(F)F)cc2)C1]

5-(4-((4-acetylmorpholin-2-yl)methoxy)phenyl)-2-oxo-6-(trifluoromethyl)-1,2-dihydropyridine-3-carboxamide (188) was made using step 2 in the procedure used to make 5-(4-((3-nitrobenzyl)oxy)phenyl)-2-oxo-6-(trifluoromethyl)-1,2-dihydropyridine-3-carboxamide (54), except 4,4,5,5-tetramethyl-2-(4-((3-nitrobenzyl)oxy)phenyl)-1,3,2-dioxaborolane was replaced with 1-(2-((4-(4,4,5,5-tetramethyl-1,3,2-dioxaborolan-2-yl)phenoxy)methyl)morpholino)ethan-1-one. LCMS: Rt=0.82 min, m/z=440.2 [M+H].

1-(2-((4-(4,4,5,5-tetramethyl-1,3,2-dioxaborolan-2-yl)phenoxy)methyl)morpholino)ethan-1-one was obtained using the method described in step 2 of Example 52, except 4-(4-bromobenzylidene)tetrahydro-2H-pyran was replaced with 1-(2-((4-bromophenoxy)methyl)morpholino)ethan-1-one.

### 1-(2-((4-bromophenoxy)methyl)morpholino)ethan-1-one was Obtained Using the Following Procedure

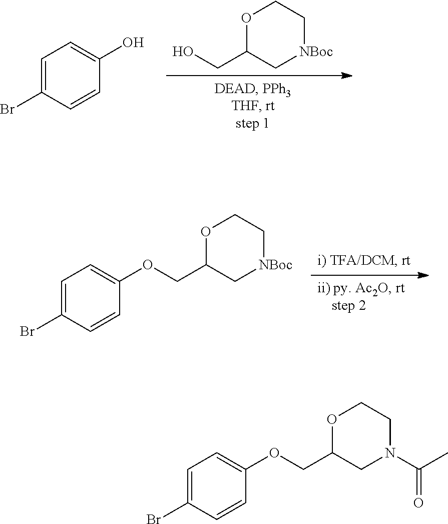

Step 1: Tert-butyl 2-((4-bromophenoxy)methyl)morpholine-4-carboxylate was prepared using the method described in step 1 in Example 167, using tert-butyl 2-(hydroxymethyl)morpholine-4-carboxylate and 4-bromophenol. LCMS t=1.19 min, m/z=316.1, 318.1 [M+H−tBu]

Step 2: To a 4 ml vial with a stirbar was added tert-butyl 2-((4-bromophenoxy)methyl)morpholine-4-carboxylate (42 mg, 0.113 mmol), followed by DCM (0.35 mL) and TFA (0.35 mL). The reaction mixture was stirred at rt for 20 mins and the volatiles were removed under a stream of air and then in vacuo. Under N₂, pyridine (0.55 mL) was added, followed by dropwise addition of acetic anhydride (0.21 mL, 2.26 mmol) and the reaction mixture stirred at rt for 45 mins. The reaction mixture was diluted with EA, washed with 1M HCl, then brine, dried over Na₂SO₄, filtered and concentrated in vacuo to give 1-(2-((4-bromophenoxy)methyl)morpholino)ethan-1-one. LCMS t=0.79, m/z=314.1, 316.1 [M+H].

### Example 189: 5-(4-(morpholin-2-ylmethoxy)phenyl)-2-oxo-6-(trifluoromethyl)-1,2-dihydropyridine-3-carboxamide (189)

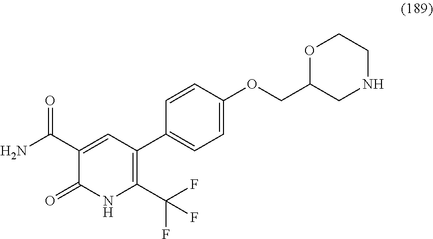 [SMILES: NC(=O)c1cc(-c2ccc(OCC3CNCCO3)cc2)c(C(F)(F)F)[nH]c1=O]

5-(4-(morpholin-2-ylmethoxy)phenyl)-2-oxo-6-(trifluoromethyl)-1,2-dihydropyridine-3-carboxamide (189) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with morpholin-2-ylmethanol. ¹H NMR (500 MHz, DMSO-d6) δ 9.02 (br. s, 2H), 8.46 (br. s, 1H), 8.25 (s, 1H), 8.15 (br. s, 1H), 7.30-7.23 (m, 2H), 7.10-7.02 (m, 2H), 4.16-3.98 (m, 4 zH), 3.76 (td, J=12.4, 2.4 Hz, 1H), 3.41-4.37 (m, 1H), 3.24 (d, J=13.3 Hz, 1H), 3.10-2.94 (m, 2). LCMS: Rt=0.72 min, m/z=398.2 [M+H].

### Example 190: tert-butyl 2-((4-(5-carbamoyl-6-oxo-2-(trifluoromethyl)-1,6-dihydropyridin-3-yl)phenoxy)methyl)morpholine-4-carboxylate (190)

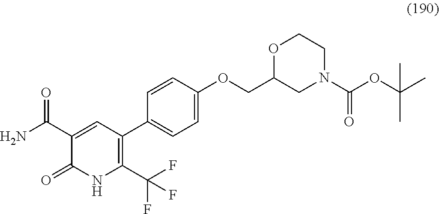 [SMILES: CC(C)(C)OC(=O)N1CCOC(COc2ccc(-c3cc(C(N)=O)c(=O)[nH]c3C(F)(F)F)cc2)C1]

Tert-butyl 2-((4-(5-carbamoyl-6-oxo-2-(trifluoromethyl)-1,6-dihydropyridin-3-yl)phenoxy)methyl)morpholine-4-carboxylate (190) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with tert-butyl 2-(hydroxymethyl)morpholine-4-carboxylate. LCMS: Rt=0.91 min, m/z=442.3 [M+H].

### Example 191: 5-(4-((4-(isopropylsulfonyl)morpholin-2-yl)methoxy)phenyl)-2-oxo-6-(trifluoromethyl)-1,2-dihydropyridine-3-carboxamide (191)

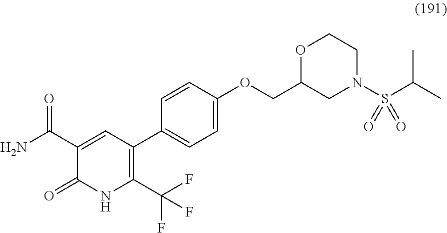 [SMILES: CC(C)S(=O)(=O)N1CCOC(COc2ccc(-c3cc(C(N)=O)c(=O)[nH]c3C(F)(F)F)cc2)C1]

5-(4-((4-(isopropylsulfonyl)morpholin-2-yl)methoxy)phenyl)-2-oxo-6-(trifluoromethyl)-1,2-dihydropyridine-3-carboxamide (191) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-(isopropylsulfonyl)morpholin-2-yl)methanol. ¹H NMR (500 MHz, DMSO-d6) δ 13.66 (br. s, 1H), 8.46 (br. s, 1H), 8.27 (s, 1H), 8.17 (br. s, 1H), 7.27 (d, J=8.4 Hz, 2H), 7.04 (d, J=8.4 Hz, 2H), 4.08 (d, J=5.1 Hz, 2H), 3.94 (d, J=11.4 Hz, 1H), 3.84-3.75 (m, 1H), 3.68 (d, J=12.5 Hz, 1H), 3.54 (td, J=11.4, 2.4, 1H), 3.48 (d, J=12.5 Hz, 1H), 3.44-3.37 (m, 1H), 3.04 (td, J=12.0, 3.2 Hz, 1H), 2.97-2.91 (m, 1H), 1.25 (d, J=6.8 Hz, 6H). LCMS: Rt=0.79 min, m/z=504.3 [M+H].

### Example 192: 5-(4-((4-(2,2-difluoroethyl)morpholin-2-yl)methoxy)phenyl)-2-oxo-6-(trifluoromethyl)-1,2-dihydropyridine-3-carboxamide (192)

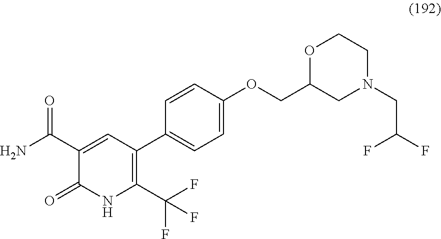 [SMILES: NC(=O)c1cc(-c2ccc(OCC3CN(CC(F)F)CCO3)cc2)c(C(F)(F)F)[nH]c1=O]

5-(4-((4-(2,2-difluoroethyl)morpholin-2-yl)methoxy)phenyl)-2-oxo-6-(trifluoromethyl)-1,2-dihydropyridine-3-carboxamide (192) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-(2,2-difluoroethyl)morpholin-2-yl)methanol. LCMS: Rt=0.57 min, m/z=462.3 [M+H].

### Example 193: 5-(4-((4-(2-methoxyethyl)morpholin-2-yl)methoxy)phenyl)-2-oxo-6-(trifluoromethyl)-1,2-dihydropyridine-3-carboxamide (193)

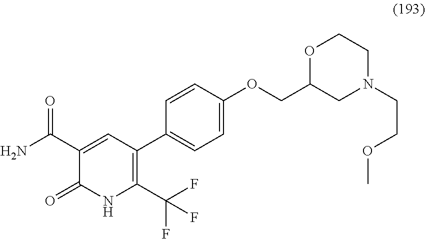 [SMILES: COCCN1CCOC(COc2ccc(-c3cc(C(N)=O)c(=O)[nH]c3C(F)(F)F)cc2)C1]

5-(4-((4-(2-methoxyethyl)morpholin-2-yl)methoxy)phenyl)-2-oxo-6-(trifluoromethyl)-1,2-dihydropyridine-3-carboxamide (193) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-(2-methoxyethyl)morpholin-2-yl)methanol. ¹H NMR (500 MHz, DMSO-d6) δ 13.66 (br. s, 1H), 10.36 (br. s, 1H), 8.46 (s, 1H), 8.27 (s, 1H), 8.16 (s, 1H), 7.29 (d, J=8.3 Hz, 2H), 7.05 (d, J=8.3 Hz, 2H), 4.20-4.03 (m, 4H), 3.84 (t, J=12.5 Hz, 1H), 3.70 (t, J=4.9 Hz, 2), 3.64 (d, J=12.5 Hz, 1H), 3.48 (d, J=12.5 Hz, 1H), 3.39 (s, 2H), 3.33 (s, 3H), 3.11 (q, J=12.3, 2H). LCMS: Rt=0.72 min, m/z=456.2 [M+H].

### Example 194: 5-(4-((4-(cyclopropylmethyl)morpholin-2-yl)methoxy)phenyl)-2-oxo-6-(trifluoromethyl)-1,2-dihydropyridine-3-carboxamide (194)

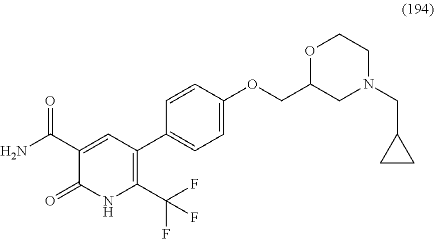 [SMILES: NC(=O)c1cc(-c2ccc(OCC3CN(CC4CC4)CCO3)cc2)c(C(F)(F)F)[nH]c1=O]

5-(4-((4-(cyclopropylmethyl)morpholin-2-yl)methoxy)phenyl)-2-oxo-6-(trifluoromethyl)-1,2-dihydropyridine-3-carboxamide (194) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 2-((4-bromophenoxy)methyl)-4-(cyclopropylmethyl)morpholine. ¹H NMR (500 MHz, DMSO-d6) δ 13.58 (br. S, 1H), 10.28 (br. S, 1H), 8.45 (s, 1H), 8.26 (s, 1H), 8.16 (s, 1H), 7.29 (d, J=8.3 Hz, 2H), 7.05 (d, J=8.3 Hz, 2H), 4.22-4.02 (m, 4H), 3.83 (t, J=12.4 Hz, 1H), 3.70 (d, J=12.4 Hz, 1H), 3.54 (d, J=12.4 Hz, 2H), 3.17-2.98 (m, 5H), 1.15-1.04 (m, 1H), 0.72-0.60 (m, 2H), 0.45-0.31 (m, 2H). LCMS: Rt=0.59 min, m/z=452.3 [M+H].

### 2-((4-bromophenoxy)methyl)-4-(cyclopropylmethyl)morpholine was Obtained Using the Following Procedure

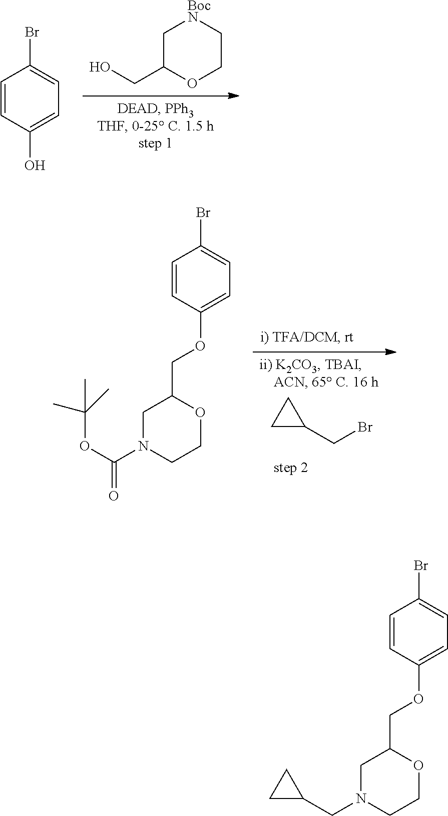

Step 1: To a 20 mL oven dried vial with stirbar was added tert-butyl 2-(hydroxymethyl)morpholine-4-carboxylate (0.50 g, 2.301 mmol), 4-Bromophenol (0.418 g, 2.416 mmol) and triphenylphosphine (0.724 g, 2.76 mmol) and the reaction vial was then sealed with a septa topped cap and purged with vac/N₂ (×3). THF (11.51 mL) was added and the reaction mixture was cooled to ° 0 C in an ice bath. DEAD (0.419 ml, 2.65 mmol) was then added dropwise and the reaction stirred at 0° C. for 5 mins before warming to rt and stirring for 1.5 h. The reaction mixture was then concentrated in vacuo and purified by silica gel chromatography (ISCO, load as a slurry in minimum DCM) 0-50% EtOAc:Hept. (product elutes at ˜30% EtOAc) to give tert-butyl 2-((4-bromophenoxy)methyl)morpholine-4-carboxylate.

Step 2: To a 4 m L vial with stirbar was added tert-butyl 2-((4-bromophenoxy)methyl)morpholine-4-carboxylate (50 mg, 0.134 mmol) followed by DCM (0.41 mL) and trifluoroacetic acid (414 μl, 5.37 mmol) and the reaction mixture stirred for 30 mins. The mixture was then concentrated and Tetrabutylammonium iodide (4.96 mg, 0.013 mmol) and potassium carbonate (74.3 mg, 0.537 mmol) were added, the reaction vial was sealed with a septa-top cap and purged with vac/N₂ (×3). Acetonitrile (448 μL) was added, followed by (bromomethyl)cyclopropane (15.43 μL, 0.161 mmol) and the reaction mixture warmed to 65° C. and stirred vigorously overnight. The reaction mixture was diluted with brine and extracted with EtOAc (×3). The organic layers were combined and dried over Na₂SO₄, filtered and concentrated to give 2-((4-bromophenoxy)methyl)-4-(cyclopropylmethyl)morpholine which was used without further purification.

### Example 195: 5-(4-((1,4-oxazepan-2-yl)methoxy)phenyl)-2-oxo-6-(trifluoromethyl)-1,2-dihydropyridine-3-carboxamide (195)

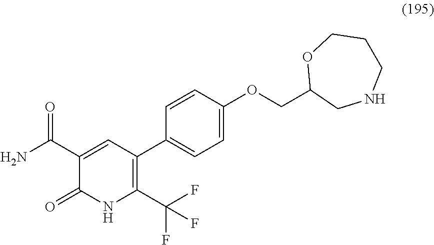 [SMILES: NC(=O)c1cc(-c2ccc(OCC3CNCCCO3)cc2)c(C(F)(F)F)[nH]c1=O]

5-(4-((1,4-oxazepan-2-yl)methoxy)phenyl)-2-oxo-6-(trifluoromethyl)-1,2-dihydropyridine-3-carboxamide (195) was made using a procedure similar to that used to make 5-(4-((1-acetylpiperidin-4-ylmethoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (1,4-oxazepan-2-yl)methanol. ¹H NMR (500 MHz, DMSO-d6) δ 13.61 (br. s, 1H), 8.90 (br. s, 2H), 8.44 (s, 1H), 8.26 (s, 1H), 8.16 (s, 1H), 7.36-7.08 (m, 2H), 7.10-6.93 (m, 2H), 4.19 (dtd, J=10.0, 5.1, 3.1 Hz, 1H), 4.13-4.03 (m, 2H), 3.97 (dt, J=11.7, 5.7 Hz, 1H), 3.70 (ddd, J=12.1, 6.7, 5.1 Hz, 1H), 3.57-3.30 (m, 2H), 3.28-3.11 (m, 2H), 2.01 (br. s, 2H). LCMS: Rt=0.96 min, m/z=412.2 [M+H].

### Example 196: 5-(4-((4-(oxetan-3-yl)-1,4-oxazepan-2-yl)methoxy)phenyl)-2-oxo-6-(trifluoromethyl)-1,2-dihydropyridine-3-carboxamide (196)

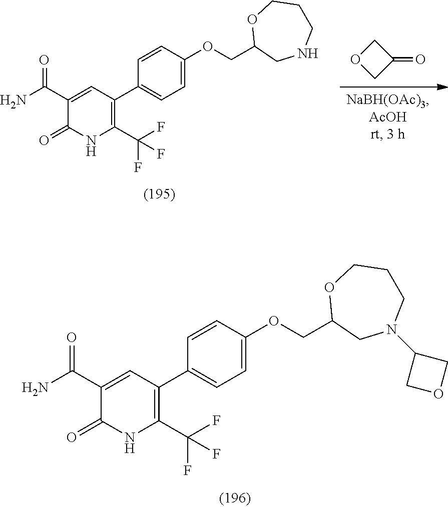

Acetic acid (0.3 μL) and oxetan-3-one (7.3 μL, 0.11 mmol) were added to a solution of 5-(4-((1,4-oxazepan-2-yl)methoxy)phenyl)-2-oxo-6-(trifluoromethyl)-1,2-dihydropyridine-3-carboxamide (195) (15 mg, 0.029 mmol) and NaBH(OAc)₃ (30.3 mg, 0.14 mmol) in DCM (0.57 mL) and the reaction mixture was stirred at rt for 3 h. The reaction mixture was concentrated and purified by reverse phase HPLC to give 5-(4-((4-(oxetan-3-yl)-1,4-oxazepan-2-yl)methoxy)phenyl)-2-oxo-6-(trifluoromethyl)-1,2-dihydropyridine-3-carboxamide (196). LCMS: Rt=0.59 min, m/z=488.2 [M+H].

### Example 197: 5-(4-((4-(isopropylsulfonyl)-1,4-oxazepan-2-yl)methoxy)phenyl)-2-oxo-6-(trifluoromethyl)-1,2-dihydropyridine-3-carboxamide (197)

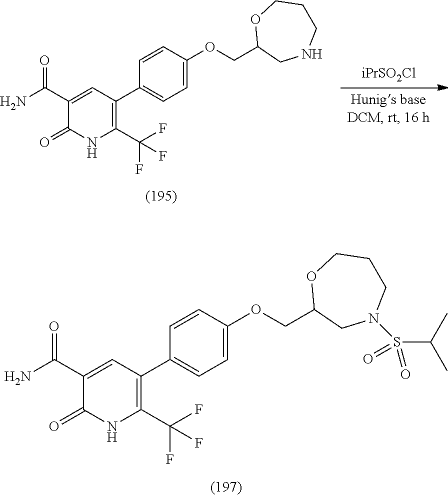

Isopropylsulfonyl chloride (3.2 μL, 0.29 mmol) was added to a solution of 5-(4-((1,4-oxazepan-2-yl)methoxy)phenyl)-2-oxo-6-(trifluoromethyl)-1,2-dihydropyridine-3-carboxamide (195) (15 mg, 0.029 mmol) and Hunig's base (50 μL, 0.29 mmol) in DCM (0.29 mL) and the reaction mixture was stirred at rt for 18 h. The reaction was quenched with pH=4 phosphate buffer, concentrated and purified by reverse phase HPLC to give 5-(4-((4-(isopropylsulfonyl)-1,4-oxazepan-2-yl)methoxy)phenyl)-2-oxo-6-(trifluoromethyl)-1,2-dihydropyridine-3-carboxamide (197). LCMS: Rt=0.83 min, m/z=518.2 [M+H].

### Example 198: 5-(4-((4-(isobutylsulfonyl)-1,4-oxazepan-2-yl)methoxy)phenyl)-2-oxo-6-(trifluoromethyl)-1,2-dihydropyridine-3-carboxamide (198)

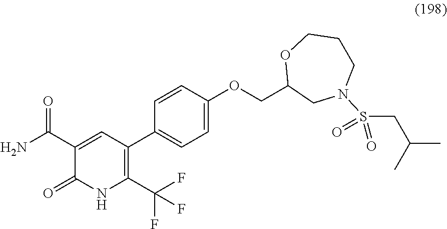 [SMILES: CC(C)CS(=O)(=O)N1CCCOC(COc2ccc(-c3cc(C(N)=O)c(=O)[nH]c3C(F)(F)F)cc2)C1]

5-(4-((4-(isobutylsulfonyl)-1,4-oxazepan-2-yl)methoxy)phenyl)-2-oxo-6-(trifluoromethyl)-1,2-dihydropyridine-3-carboxamide (198) was obtained using the method described the synthesis of 5-(4-((4-(isopropylsulfonyl)-1,4-oxazepan-2-yl)methoxy)phenyl)-2-oxo-6-(trifluoromethyl)-1,2-dihydropyridine-3-carboxamide (197), except isopropylsulfonyl chloride was replaced with 2-methylpropane-1-sulfonyl chloride. ¹H NMR (500 MHz, DMSO-d6) δ 13.64 (br. s, 1H), 8.46 (br. s, 1H), 8.28 (s, 1H), 8.18 (br. s, 1H), 7.34-7.21 (m, 2H), 7.09-6.97 (m, 2H), 4.08-3.98 (m, 3H), 3.93 (ddd, J=8.8, 5.4, 3.3 Hz, 1H), 3.74 (dd, J=14.1, 3.2 Hz, 1H), 3.65 (ddd, J=12.7, 8.4, 4.6 Hz, 1H), 3.52 (dt, J=12.7, 6.0 Hz, 1H), 3.31-3.22 (m, 2H), 3.05-2.92 (m, 2H), 2.10 (dp, J=13.3, 6.5 Hz, 1H), 1.86 (dq, J=6.8, 5.0 Hz, 2H), 1.03 (dd, J=6.7, 1.4 Hz, 6H). LCMS: Rt=0.89 min, m/z=532.2 [M+H].

### Example 199: 5-(4-(2-morpholino-2-oxoethoxy)phenyl)-2-oxo-6-(trifluoromethyl)-1,2-dihydropyridine-3-carboxamide (199)

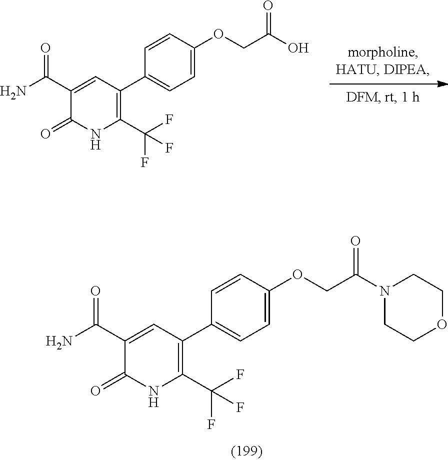 [SMILES: NC(=O)c1cc(-c2ccc(OCC(=O)O)cc2)c(C(F)(F)F)[nH]c1=O ; "DFM, rt, 1 h" ; "morpholine,HATU, DIPEA," ; NC(=O)c1cc(-c2ccc(OCC(=O)N3CCOCC3)cc2)c(C(F)(F)F)[nH]c1=O]

In a 5 mL vial was added 2-(4-(5-carbamoyl-6-oxo-2-(trifluoromethyl)-1,6-dihydropyridin-3-yl)phenoxy)acetic acid (10. mg, 0.028 mmol) in DMF (56 μl). Morpholine (2.70 μl, 0.031 mmol) was added, followed by DIPEA (25 μl, 0.14 mmol) and dropwise addition of HATU as a 0.1M solution in DMF. The reaction was stirred at rt for 1 h. Purification by reverse phase HPLC gave 5-(4-(2-morpholino-2-oxoethoxy)phenyl)-2-oxo-6-(trifluoromethyl)-1,2-dihydropyridine-3-carboxamide (199). ¹H NMR (500 MHz, DMSO-d6) δ 13.65 (br. s, 1H), 8.46 (br. s, 1H), 8.28 (s, 1H), 8.18 (br. s, 1H), 7.29-7.22 (m, 2H), 7.01 (d, J=8.7 Hz, 2H), 4.90 (s, 2H), 3.71-3.35 (m, 8H). LCMS: Rt=0.57 min, m/z=426.3 [M+H].

2-(4-(5-carbamoyl-6-oxo-2-(trifluoromethyl)-1,6-dihydropyridin-3-yl)phenoxy)acetic acid was obtained using step 1 in the procedure used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 2-bromoacetic acid. LCMS: Rt=0.59 min, m/z—357.2 [M+H].

### Example 200: 5-(4-(2-(butylamino)-2-oxoethoxy)phenyl)-2-oxo-6-(trifluoromethyl)-1,2-dihydropyridine-3-carboxamide (200)

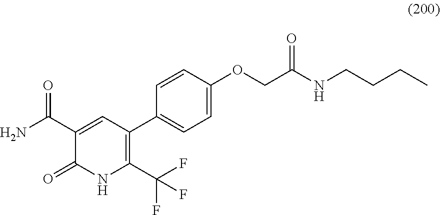 [SMILES: CCCCNC(=O)COc1ccc(-c2cc(C(N)=O)c(=O)[nH]c2C(F)(F)F)cc1]

5-(4-(2-(butylamino)-2-oxoethoxy)phenyl)-2-oxo-6-(trifluoromethyl)-1,2-dihydropyridine-3-carboxamide (200) was obtained using the method described for the synthesis of 5-(4-(2-morpholino-2-oxoethoxy)phenyl)-2-oxo-6-(trifluoromethyl)-1,2-dihydropyridine-3-carboxamide (199) except morpholine was replaced with butan-1-amine. ¹H NMR (500 MHz, DMSO-d6) δ 13.64 (br. s, 1H), 8.46 (br. s, 1H), 8.27 (s, 1H), 8.17 (br. s, 1H), 8.09 (t, J=5.9 Hz, 1H), 7.29 (d, J=8.6 Hz, 2H), 7.03 (d, J=8.6 Hz, 2H), 4.52 (s, 2H), 3.13 (q, J=6.7 Hz, 2H), 1.50-1.36 (m, 2H), 1.26 (h, J=7.3 Hz, 2H), 0.86 (t, J=7.3 Hz, 3H). LCMS: Rt=0.77 min, m/z=412.3 [M+H].

### Example 201: 2-oxo-5-(4-(2-oxo-2-(pyridin-3-ylamino)ethoxy)phenyl)-6-(trifluoromethyl)-1,2-dihydropyridine-3-carboxamide (201)

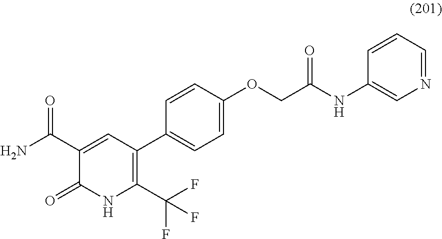 [SMILES: NC(=O)c1cc(-c2ccc(OCC(=O)Nc3cccnc3)cc2)c(C(F)(F)F)[nH]c1=O]

2-oxo-5-(4-(2-oxo-2-(pyridin-3-ylamino)ethoxy)phenyl)-6-(trifluoromethyl)-1,2-dihydropyridine-3-carboxamide (201) was obtained using the method described for the synthesis of 5-(4-(2-morpholino-2-oxoethoxy)phenyl)-2-oxo-6-(trifluoromethyl)-1,2-dihydropyridine-3-carboxamide (199) except morpholine was replaced with pyridin-3-amine. ¹H NMR (500 MHz, DMSO-d6) δ 10.50 (s, 1H), 8.89 (d, J=2.5 Hz, 1H), 8.45 (s, 1H), 8.37 (d, J=4.8 Hz, 1H), 8.27 (s, 1H), 8.24-8.11 (m, 2H), 7.50 (dd, J=8.4, 4.8 Hz, 1H), 7.33-7.27 (m, 2H), 7.13-7.08 (m, 2H), 4.82 (s, 2H). LCMS: Rt=0.54 min, m/z=433.3 [M+H].

### Example 202 and Example 203: 5-(4-((4-methoxytetrahydro-2H-pyran-2-yl)methoxy)phenyl)-2-oxo-6-(trifluoromethyl)-1,2-dihydropyridine-3-carboxamide, (SFC Peak 1) (202) and 5-(4-((4-methoxytetrahydro-2H-pyran-2-yl)methoxy)phenyl)-2-oxo-6-(trifluoromethyl)-1,2-dihydropyridine-3-carboxamide, (SFC Peak 2) (203)

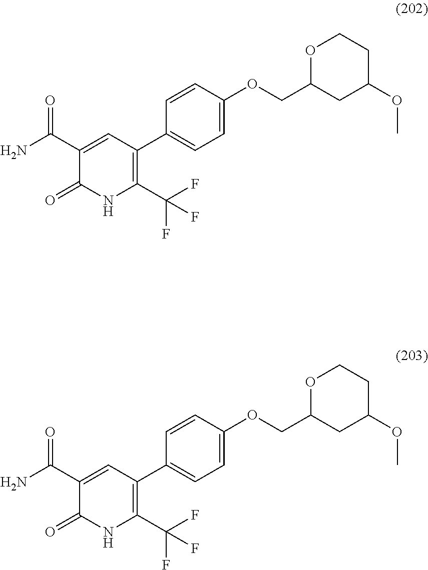 [SMILES: COC1CCOC(COc2ccc(-c3cc(C(N)=O)c(=O)[nH]c3C(F)(F)F)cc2)C1 ; COC1CCOC(COc2ccc(-c3cc(C(N)=O)c(=O)[nH]c3C(F)(F)F)cc2)C1]

5-(4-((4-methoxytetrahydro-2H-pyran-2-yl)methoxy)phenyl)-2-oxo-6-(trifluoromethyl)-1,2-dihydropyridine-3-carboxamide, (SFC peak 1) (202) and 5-(4-((4-methoxytetrahydro-2H-pyran-2-yl)methoxy)phenyl)-2-oxo-6-(trifluoromethyl)-1,2-dihydropyridine-3-carboxamide, (SFC peak 2) (203) were obtained after SFC purification of the product made using step 2 in the procedure used to make 5-(4-((3-nitrobenzyl)oxy)phenyl)-2-oxo-6-(trifluoromethyl)-1,2-dihydropyridine-3-carboxamide (54), except 4,4,5,5-tetramethyl-2-(4-((3-nitrobenzyloxy)phenyl)-1,3,2-dioxaborolane was replaced with 2-(4-((4-methoxytetrahydro-2H-pyran-2-yl)methoxy)phenyl-4,4,5,5-tetramethyl-1,3,2-dioxaborolane and the diastereomers separated by chiral SFC.

5-(4-((4-methoxytetrahydro-2H-pyran-2-yl)methoxy)phenyl)-2-oxo-6-(trifluoromethyl)-1,2-dihydropyridine-3-carboxamide, (SFC peak 1) (202): ¹H NMR (400 MHz, MeOD-d4) δ 8.28 (br. s, 1H), 7.24 (br. d, J=8.1 Hz, 2H), 7.01 (br. d, J=8.1 Hz, 2H), 4.13-3.96 (m, 3H), 3.82-3.69 (m, 1H), 3.56-3.44 (m, 2H), 3.39 (s, 3H), 2.24-1.97 (m, 2H), 1.50-1.22 (m, 2H); LCMS: Rt=0.74 min, m/z=427.0 [M+H]; Chiral HPLC: Rt=2.42 min, d.e value=98%.

5-(4-((4-methoxytetrahydro-2H-pyran-2-yl)methoxy)phenyl)-2-oxo-6-(trifluoromethyl)-1,2-dihydropyridine-3-carboxamide, (SFC peak 2) (203): ¹H NMR (400 MHz, MeOD-d4) δ 8.08 (s, 1H), 7.18 (d, J=8.6 Hz, 2H), 6.95 (d, J=8.6 Hz, 2H), 4.10-4.00 (m, 3H), 3.80-3.72 (m, 1H), 3.56-3.46 (m, 2H), 3.39 (s, 3H), 2.21-1.96 (m, 2H), 1.48-1.25 (m, 2H); LCMS: Rt=0.74 min, m/z=427.0 [M+H]; Chiral HPLC: Rt=3.17 min, d.e value=97%.

### 2-(4-((4-methoxytetrahydro-2H-pyran-2-yl)methoxy)phenyl)-4,4,5,5-tetramethyl-1,3,2-dioxaborolane was Obtained Using the Following Procedure

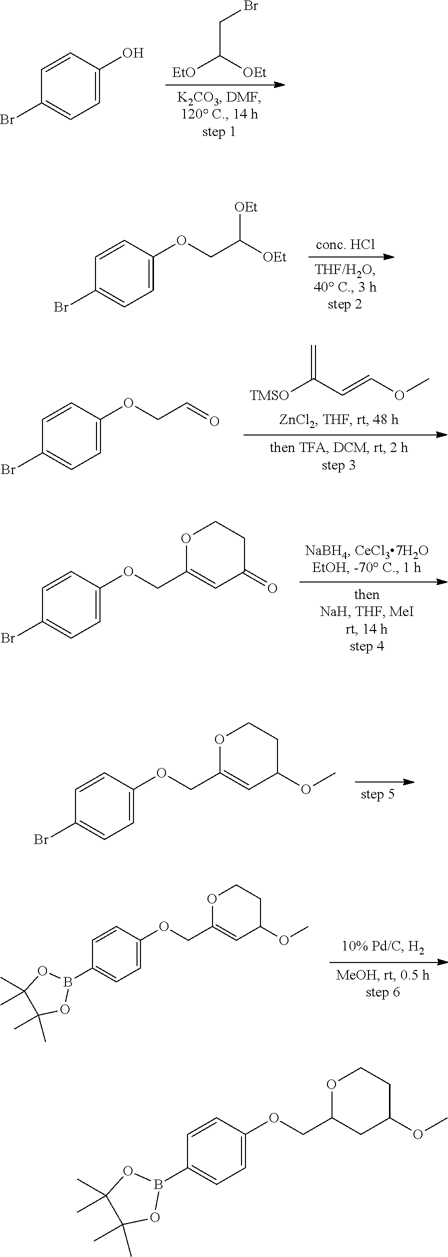

Step 1: To a solution of 4-bromophenol (10 g, 57.8 mmol) in DMF (200 mL) was added K₂CO₃ (12 g, 86.7 mmol) followed by 2-bromo-1,1-diethoxyethane (17 g, 86.7 mmol). Then the mixture was stirred at 120° C. for 14 hr. The reaction mixture was diluted with water (1 L) and extracted with EA (300 mL×2), the organic layers were combined and dried over anhydrous Na₂SO₄, filtered and concentrated to give the crude product which was purified by silica gel flash column chromatography (PE/EA=50/1) to give 1-bromo-4-(2,2-diethoxyethoxy)benzene. ¹H NMR (400 MHz, CDCl₃) δ 7.39-7.33 (m, 2H), 6.84-6.76 (m, 2H), 4.82 (t, J=5.2 Hz, 1H), 3.97 (d, J=5.1 Hz, 2H), 3.82-3.55 (m, 4H), 1.29-1.21 (m, 6H).

Step 2: To a stirred solution of 1-bromo-4-(2,2-diethoxyethoxy)benzene (15 g, 51.9 mmol) in THF/H2O (1:1, 200 mL) was added conc. HCl (34.6 mL) and the reaction was warmed to 40° C. and stirred for 3 h. The reaction mixture was evaporated to remove the EtOH and the resulting residue extracted with DCM (2×20 mL). The organic layers were combined and dried over anhydrous Na₂SO₄, filtered, and evaporated to dryness under reduced pressure. The residue was purified by silica gel chromatography (PE/EA=8/1) to give 2-(4-bromophenoxy)acetaldehyde. LCMS: Rt=0.63 min, m/z=214.8, 216.8 [M+H]; ¹H NMR (400 MHz, CDCl₃) δ=9.83 (s, 1H), 7.43-7.39 (m, 2H), 6.80-6.76 (m, 2H), 4.55 (s, 2H).

Step 3: To a solution of 2-(4-bromophenoxy)acetaldehyde (4.0 g, 13.6 mmol) and (E)-((4-methoxybuta-1,3-dien-2-yl)oxy)trimethylsilane (2.57 g, 14.9 mmol) in THF (16 mL) was added dropwise ZnCl₂ (1M in THF, 14.9 mL, 14.9 mmol) at 0° C. and the reaction mixture stirred under N₂ at 25° C. for 48 h. The reaction mixture was diluted with water (160 mL) and extracted with EA (100 mL×2). The combined organic layers were dried and concentrated to give crude product which was dissolved in DCM (16 mL). TFA (8 mL) was added and the reaction was stirred at rt for 2 h. The reaction mixture was diluted with water (100 mL), extracted with DCM (100 mL×2), and the combined organic layers were adjusted to pH=10 with saturated NaHCO₃ (aq., 200 mL). The organic layer was dried and concentrated to give a residue which was purified by silica gel 12 chromotography (PE/EA=4/1) to give 6-((4-bromophenoxy)methyl)-2,3-dihydro-4H-pyran-4-one. LCMS: Rt=0.79 min, m/z=282.9, 284.9, [M+H]; ¹H NMR (400 MHz, CDCl₃) δ=7.44-7.39 (m, 3H), 6.85-6.79 (m, 2H), 5.48 (dd, J=1.0, 6.0 Hz, 1H), 4.84-4.73 (m, 1H), 4.19 (dd, J=1.8, 4.4 Hz, 2H), 4.22-4.17 (m, 1H), 2.85 (dd, J=14.0, 16.8 Hz, 1H), 2.55 (ddd, J=1.0, 3.6, 16.8 Hz, 1H)

Step 4: To a solution of 6-((4-bromophenoxy)methyl)-2,3-dihydro-4H-pyran-4-one (2.0 g, 7.06 mmol) in EtOH (40 mL) at room temperature was added CeCl₃·7H₂O (2.6 g, 7.06 mmol). After the atmosphere was replaced with nitrogen, the mixture was cooled to −70° C. and sodium borohydride (534 mg, 14.1 mmol) was added. The mixture was then stirred at −70° C. for 1 h. A saturated aqueous NaHCO₃ solution (50 mL) was added and the mixture warmed to room temperature before filtering through Celite®. The filtrate was extracted with DCM (50 mL×3) and the combined organic layers were washed with brine and then dried over Na₂SO₄, filtered and the solvent was evaporated to give crude 6-((4-bromophenoxy)methyl)-4-methoxy-3,4-dihydro-2H-pyran which was used to the next step without further purification.

Step 5: 2-(4-((4-methoxy-3,4-dihydro-2H-pyran-6-yl)methoxy)phenyl)-4,4,5,5-tetramethyl-1,3,2-dioxaborolane was obtained using step 1 in the procedure used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 6-((4-bromophenoxy)methyl)-4-methoxy-3,4-dihydro-2H-pyran. LCMS: Rt=0.927 min, m/z=315.1 [M−MeOH+H]; ¹H NMR (400 MHz, CDCl₃) δ=7.75 (d, J=8.6 Hz, 2H), 6.94-6.90 (m, 2H), 6.44 (dd, J=0.9, 6.3 Hz, 1H), 4.94-4.88 (m, 1H), 4.46-4.34 (m, 1H), 4.24 (dd, J=6.5, 10.1 Hz, 1H), 4.09-4.01 (m, 2H), 3.37 (s, 3H), 2.31-2.22 (m, 1H), 1.90 (ddd, J=7.7, 9.6, 13.4 Hz, 1H), 1.34 (s, 12H).

Step 6: To a solution of 2-(4-((4-methoxy-3,4-dihydro-2H-pyran-6-yl)methoxy)phenyl)-4,4,5,5-tetramethyl-1,3,2-dioxaborolane (700 mg, 1.54 mmol) in MeOH (14 mL) at 25° C. was added 10% Pd/C (140 mg, 0.31 mmol) under H₂ and the reaction mixture stirred at 25° C. for 0.5 h. The mixture was filtered and concentrated to give 2-(4-((4-methoxytetrahydro-2H-pyran-2-yl)methoxy)phenyl))-4,4,5,5-tetramethyl-1,3,2-dioxaborolane which was used without further purification. ¹H NMR (400 MHz, CDCl3) δ=7.74 (d, J=8.6 Hz, 2H), 6.95-6.86 (m, 2H), 4.17-4.03 (m, 2H), 3.96 (dd, J=4.0, 9.9 Hz, 1H), 3.77-3.69 (m, 1H), 3.53-3.48 (m, 2H), 3.47-3.41 (m, 1H), 3.40-3.36 (m, 3H), 2.17-2.10 (m, 1H), 2.03-1.97 (m, 1H), 1.58-1.47 (m, 1H), 1.33 (s, 12H); LCMS: Rt=0.97 min, m/z=371.2 [M+Na].

### Example 204: 5-(4-(((2S,5S)-5-(hydroxymethyl)-1,4-dioxan-2-yl)methoxy)phenyl)-2-oxo-6-(trifluoromethyl)-1,2-dihydropyridine-3-carboxamide (204)

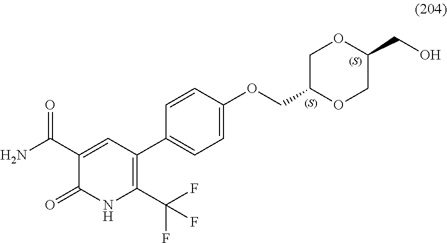

5-(4-(((2S,5S)-5-(hydroxymethyl)-1,4-dioxan-2-yl)methoxy)phenyl)-2-oxo-6-(trifluoromethyl)-1,2-dihydropyridine-3-carboxamide (204) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 2-((benzyloxy)methyl)-5-(iodomethyl)-1,4-dioxane. ¹H NMR (400 MHz, DMSO-d6) δ 10.04 (br. s, 1H), 8.43-8.17 (m, 1H), 7.94 (br.s, 1H), 7.47-7.26 (m, 1H), 7.22-7.12 (m, 2H), 7.04-6.88 (m, 2H), 4.24-4.08 (m, 2H), 4.05-3.95 (m, 2H), 3.94-3.87 (m, 2H), 3.86-3.78 (m, 2H), 3.72 (m, 4H); LCMS: Rt=0.61 min, m/z=429.1 [M+H].

### 2-((benzyloxy)methyl)-5-(iodomethyl)-1,4-dioxane was Obtained Using the Following Procedure

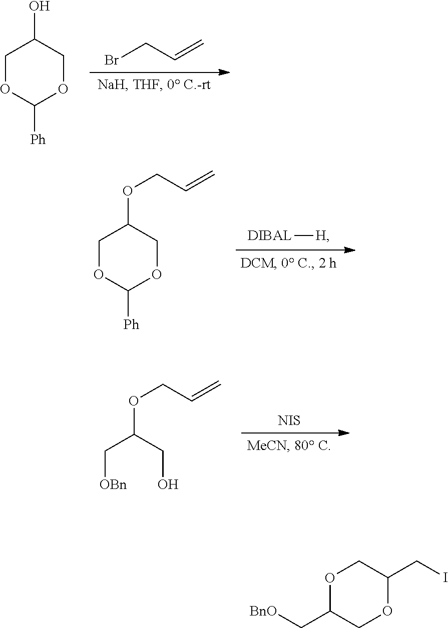

Step 1: To a solution of 2-phenyl-1,3-dioxan-5-ol (5.0 g, 27.8 mmol) in THF (50 mL) was added NaH (2.2 g, 55.5 mmol) batchwise at 0° C. The mixture was stirred at 0° C. for 0.5 h. Allyl bromide (6.7 g, 55.5 mmol) was added and the reaction mixture was stirred at 25° C. for 1.5 hrs. The reaction was slowly quenched by addition of water and the reaction mixture was then extracted with EA (50 mL×3). The organic layers were combined, washed with brine (10 mL×3), dried over anhydrous Na₂SO₄, filtered and concentrated in vacuo. The residue was purified by silica gel chromatography (PE/EA=20/1) to give 5-(allyloxy)-2-phenyl-1,3-dioxane. TLC (PE/EA=5:1) Rf=0.4. ¹H NMR (400 MHz, CHLOROFORM-d) δ 7.52-7.40 (m, 2H), 7.26 (s, 3H), 5.96-5.83 (m, 1H), 5.48 (s, 1H), 5.33-5.20 (m, 1H), 5.19-5.07 (m, 1H), 4.25 (s, 2H), 4.08 (d, J=5.6 Hz, 2H), 4.02-3.95 (m, 2H), 3.31-3.24 (m, 1H).

Step 2: DIBAL-H (1 M solution in DCM, 45.4 mL, 45.4 mmol) was added slowly to a solution of 5-(allyloxy)-2-phenyl-1,3-dioxane (5.0 g, 22.7 mmol) in DCM (35 mL) cooled with an ice bath. After stirring at 0° C. for 2 hrs, the reaction was slowly quenched by addition of water and the reaction mixture was then extracted with EA (50 mL×3). The organic layers were combined, washed with brine (5 mL×3), dried over anhydrous Na₂SO₄, filtered and concentrated in vacuo. The residue was purified by silica gel chromatography (PE/EA=20/1) to give 2-(allyloxy)-3-(benzyloxy)propan-1-ol. TLC (PE:EA=3:1) Rf=0.3; ¹H NMR (400 MHz, CHLOROFORM-d) δ 7.41-7.29 (m, 5H), 6.02-5.88 (m, 1H), 5.31 (qd, J=1.6, 17.2 Hz, 1H), 5.21 (qd, J=1.4, 10.4 Hz, 1H), 4.57 (d, J=1.1 Hz, 2H), 4.24-4.07 (m, 2H), 3.85-3.53 (m, 6H).

Step 3: NIS (3.34 g, 14.85 mmol) was added to a solution of 2-(allyloxy)-3-(benzyloxy)propan-1-ol (1.65 g, 7.42 mmol) in dry MeCN (25 mL). After 3 hrs of stirring at 80° C. the reaction mixture was washed with a saturated Na₂SO₃ solution (30 mL) and then extracted with EA (30 mL×3). The organic layers were combined, washed with brine (10 mL), dried over anhydrous Na₂SO₄, filtered and concentrated in vacuo. The residue was purified by reverse phase chromatography to give 2-((benzyloxy)methyl)-5-(iodomethyl)-1,4-dioxane. LCMS: Rt=0.87 min, m/z=371.0 [M+Na]; ¹H NMR (400 MHz, CHLOROFORM-d) δ 7.27-7.25 (m, 5H), 4.51-4.47 (m, 2H), 4.23-3.25 (m, 10H).

### Example 205: 5-(4-((2,8-dioxa-5-azaspiro[3.5]nonan-6-yl)methoxy)phenyl)-2-oxo-6-(trifluoromethyl)-1,2-dihydropyridine-3-carboxamide (205)

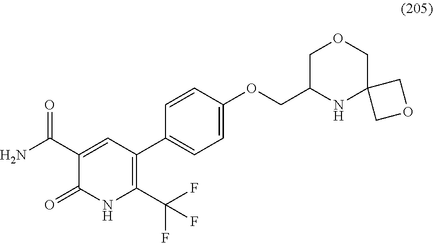 [SMILES: NC(=O)c1cc(-c2ccc(OCC3COCC4(COC4)N3)cc2)c(C(F)(F)F)[nH]c1=O]

5-(4-((2,8-dioxa-5-azaspiro[3.5]nonan-6-yl)methoxy)phenyl)-2-oxo-6-(trifluoromethyl)-1,2-dihydropyridine-3-carboxamide (205) was made using steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 6-((4-bromophenoxy)methyl)-2,8-dioxa-5-azaspiro[3.5]nonane. ¹H NMR (400 MHz, DMSO-d6) δ 8.78 (s, 1H), 8.22 (s, 1H), 8.03 (s, 1H), 7.26 (d, J=8.8 Hz, 2H), 7.03 (d, J=8.8 Hz, 2H), 4.52-4.45 (m, 2H), 4.30 (d, J=6.4 Hz, 1H), 4.22 (d, J=6.4 Hz, 1H), 3.99 (d, J=10.8 Hz, 1H), 3.87 (d, J=6.0 Hz, 2H), 3.81 (m, 1H), 3.25 (m, 2H), 3.12 (m, 1H); LCMS: Rt=0.55 min, m/z=440.2 [M+H].

### 6-((4-bromophenoxy)methyl)-2,8-dioxa-5-azaspiro[3.5]nonane was Obtained Using the Following Procedure

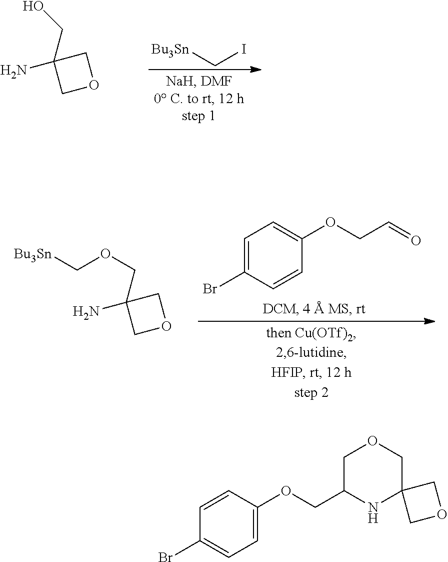

Step 1: To a solution of (3-aminooxetan-3-yl)methanol (2.3 g, 22.3 mmol) in DMF (25 mL) was added NaH (1.07 g, 26.8 mmol) at 0° C. and the reaction mixture stirred 0° C. for 1 hr. Tributyl(iodomethyl)stannane (11.5 g, 26.8 mmol) was then added at 0° C. and the reaction mixture was warmed to 25° C. and stirred for 11 h. The reaction mixture was added to an aqueous solution of NH₄Cl (100 mL) and extracted with EA (100 mL×2). The combined organic layers were dried over anhydrous Na₂SO₄, filtered and concentrated. The residue was purified by column (PE/EA=15/1 to 3/1) to give 3-(((tributylstannyl)methoxy)methyl)oxetan-3-amine. ¹H NMR (400 MHz, CDCl₃) δ 4.48 (d, J=6.8 Hz, 2H), 4.41 (d, J=6.8 Hz, 2H, 3.80 (s, 2H), 3.76 (s, 2H), 1.78 (b rs, 2H), 2.28 (s, 6H), 1.56-1.47 (m, 6H), 1.38-1.25 (m, 6H), 0.96-0.87 (m, 15H).

Step 2: To a solution of 3-(((tributylstannyl)methoxy)methyl)oxetan-3-amine (4.06 g, 10 mmol) in DCM (20 mL) at 25° C. was added 2-(4-bromophenoxy)acetaldehyde (2.15 g, 10 mmol) and MS 4A (1 g, ca. 100 mg/mmol). The reaction mixture was stirred at 25° C. for 12 hr and filtered through Celite®. The filtrate was concentrated under reduced pressure to afford the crude imine. Separately, 2,6-lutidine (1.07 g, 10 mmol) was added in one portion to a suspension of HFIP (40 mL) and anhydrous Cu(OTf)₂ (3.6 g, 10 mmol) and stirred at 25° C. for 1 hr. A solution of the imine (5.0 g, crude) in DCM (20 mL) was added in one portion and the resulting mixture was stirred at 25° C. for 11 hrs. The reaction was quenched at 25° C. with 10% aq NH₄OH (20 mL), and stirred vigorously for 15 min. The layers were separated and the aqueous layer was extracted with DCM (2×200 mL). The combined organic layers were washed with H₂O (2×100 mL) and brine (100 mL), dried over Na₂SO₄, filtered, and concentrated. The residue was purified by column (PE/EA=3/1) to give 6-((4-bromophenoxy)methyl)-2,8-dioxa-5-azaspiro[3.5]nonane. ¹H NMR (400 MHz, CDCl₃) δ 7.42-7.36 (m, 2H), 6.82-6.76 (m, 2H), 4.72 (d, J=6.4 Hz, 1H), 4.58 (m, 1H), 4.44-4.35 (m, 2H), 4.20-4.08 (m, 2H), 3.90-3.82 (m, 2H), 3.78-3.68 (m, 1H), 3.59 (m, 1H), 3.39-3.26 (m, 2H).

### Example 206: 5-(4-((5-methyl-2,8-dioxa-5-azaspiro[3.5]nonan-6-yl)methoxy)phenyl)-2-oxo-6-(trifluoromethyl)-1,2-dihydropyridine-3-carboxamide (206)

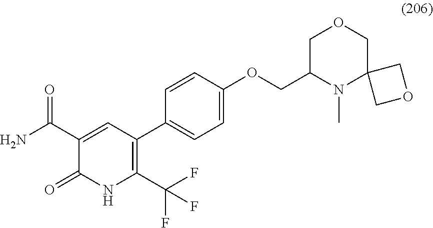 [SMILES: CN1C(COc2ccc(-c3cc(C(N)=O)c(=O)[nH]c3C(F)(F)F)cc2)COCC12COC2]

5-(4-((5-methyl-2,8-dioxa-5-azaspiro[3.5]nonan-6-yl)methoxy)phenyl)-2-oxo-6-(trifluoromethyl)-1,2-dihydropyridine-3-carboxamide (206) was made using steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 6-((4-bromophenoxy)methyl)-5-methyl-2,8-dioxa-5-azaspiro[3.5]nonane. ¹H NMR (400 MHz, DMSO-d6) δ 8.17 (s, 1H), 8.10 (s, 1H), 7.75 (s, 1H), 7.22 (d, J=8.6 Hz, 2H), 7.01 (d, J=8.6 Hz, 2H), 4.80 (d, J=6.8 Hz, 1H), 4.72 (d, J=6.8 Hz, 1H), 4.21-4.09 (m, 3H), 3.88-3.80 (m, 2H), 3.78-3.70 (m, 2H), 3.61 (m, 1H), 2.85-2.76 (m, 1H), 2.60 (s, 3H); LCMS: Rt=0.56 min, m/z=454.1 [M+H].

### 6-((4-bromophenoxy)methyl)-5-methyl-2,8-dioxa-5-azaspiro[3.5]nonane was Obtained Using the Following Procedure

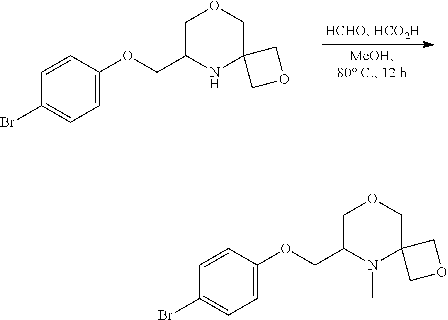 [SMILES: Brc1ccc(OCC2COCC3(COC3)N2)cc1 ; CN1C(COc2ccc(Br)cc2)COCC12COC2]

To a solution of 6-((4-bromophenoxy)methyl)-2,8-dioxa-5-azaspiro[3.5]nonane (150 mg, 0.48 mmol) and CHOOH (442 mg, 9.60 mmol) in MeOH (2 mL) was added paraformaldehyde (194 mg, 2.39 mmol) at 25° C. The mixture was stirred at 80° C. for 12 hrs. The mixture was cooled to 0° C. and then poured into ice-water (5 mL) and stirred for 15 min. The aqueous phase was extracted with ethyl acetate (10 mL×2). The combined organic phases were washed with aqueous Na₂CO₃ (5 mL) and brine (5 mL), dried over anhydrous Na2SO4, filtered and concentrated in vacuum. The residue was purified by prep-TLC (EA/MeOH=10/1) to give 6-((4-bromophenoxy)methyl)-5-methyl-2,8-dioxa-5-azaspiro[3.5]nonane. LCMS: Rt=0.57 min, m/z=328.0, 330 [M+H].

### Example 207: 2-oxo-6-(trifluoromethyl)-5-(4-((4,5,5-trimethylmorpholin-3-yl)methoxy)phenyl)-1,2-dihydropyridine-3-carboxamide (207)

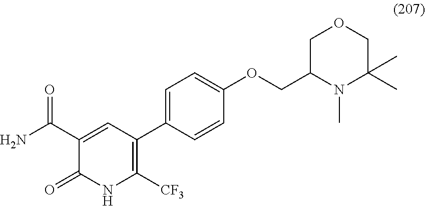 [SMILES: CN1C(COc2ccc(-c3cc(C(N)=O)c(=O)[nH]c3C(F)(F)F)cc2)COCC1(C)C]

2-oxo-6-(trifluoromethyl)-5-(4-((4,5,5-trimethylmorpholin-3-yl)methoxy)phenyl)-1,2-dihydropyridine-3-carboxamide (207) was made using steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-((4-bromophenoxy)methyl)-3,3,4-trimethylmorpholine. ¹H NMR (400 MHz, DMSO-d6) δ=9.07 (brs, 1H), 8.13 (s, 1H), 7.84 (brs, 1H), 7.23 (d, J=8.0 Hz, 2H), 7.01 (d, J=8.0 Hz, 2H), 4.12 (m, 1H), 3.93 (m, 1H), 3.84 (m, 1H), 3.39 (s, 2H), 3.31 (m 1H), 2.87 (m, 1H), 2.23 (s, 3H), 1.00 (m, 6H). LCMS: Rt=0.74 min, m/z=440.4 (M+H).

5-((4-bromophenoxy)methyl)-3,3,4-trimethylmorpholine was obtained using the procedure described for the synthesis of 6-((4-bromophenoxy)methyl)-5-methyl-2,8-dioxa-5-azaspiro[3.5]nonane except 6-((4-bromophenoxy)methyl)-2,8-dioxa-5-azaspiro[3.5]nonane was replaced with 5-((4-bromophenoxy)methyl)-3,3-dimethylmorpholine.

5-((4-bromophenoxy)methyl)-3,3-dimethylmorpholine was obtained using the procedure described for the synthesis of 6-((4-bromophenoxy)methyl)-2,8-dioxa-5-azaspiro[3.5]nonane except (3-aminooxetan-3-yl)methanol was replaced with 2-amino-2-methylpropan-1-ol.

### Example 208: 5-(4-(((5S)-4,5-dimethylmorpholin-3-yl)methoxy)phenyl)-2-oxo-6-(trifluoromethyl)-1,2-dihydropyridine-3-carboxamide (208)

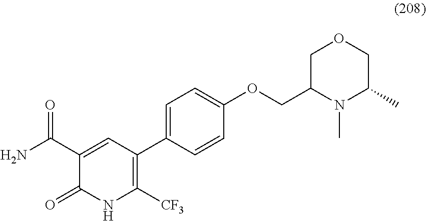 [SMILES: C[C@H]1COCC(COc2ccc(-c3cc(C(N)=O)c(=O)[nH]c3C(F)(F)F)cc2)N1C]

5-(4-(((5S)-4,5-dimethylmorpholin-3-yl)methoxy)phenyl)-2-oxo-6-(trifluoromethyl)-1,2-dihydropyridine-3-carboxamide (208) was made using steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with (5S)-3-((4-bromophenoxy)methyl)-4,5-dimethylmorpholine. ¹H NMR: (400 MHz, DMSO-d6), δ: 9.53 (br, s, 1H), 8.25 (m, 1H), 8.03 (br, s, 1H), 7.63 (br, s, 1H), 7.23 (m, 2H), 6.99 (m, 2H), 4.15 (m, 1H), 3.92 (m, 2H), 3.65 (m, 1H), 3.62 (m, 1H), 3.42 (m, 1H), 3.37 (m, 1H), 2.29 (s, 3H), 0.93 (d, J=0.64 Hz, 3H). LCMS: Rt=0.73 min, m/z=426.3. (M+H)

(5S)-3-((4-bromophenoxy)methyl)-4,5-dimethylmorpholine was obtained using the procedure described for the synthesis of 6-((4-bromophenoxy)methyl)-5-methyl-2,8-dioxa-5-azaspiro[3.5]nonane except 6-((4-bromophenoxy)methyl)-2,8-dioxa-5-azaspiro[3.5]nonane was replaced with (5S)-3-((4-bromophenoxy)methyl)-5-methylmorpholine.

(5S)-3-((4-bromophenoxy)methyl)-5-methylmorpholine was obtained using the procedure described for the synthesis of 6-((4-bromophenoxy)methyl)-2,8-dioxa-5-azaspiro[3.5]nonane except (3-aminooxetan-3-yl)methanol was replaced with (S)-2-aminopropan-1-ol.

### Example 209: 5-(4-(((3S,5S)-5-cyclopropyl-4-methylmorpholin-3-yl)methoxy)phenyl)-2-oxo-6-(trifluoromethyl)-1,2-dihydropyridine-3-carboxamide (209)

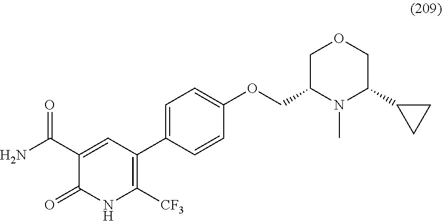 [SMILES: CN1[C@H](COc2ccc(-c3cc(C(N)=O)c(=O)[nH]c3C(F)(F)F)cc2)COC[C@@H]1C1CC1]

5-(4-(((3S,5S)-5-cyclopropyl-4-methylmorpholin-3-yl)methoxy)phenyl)-2-oxo-6-(trifluoromethyl)-1,2-dihydropyridine-3-carboxamide (209) was made using steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with (3S,5S)-3-((4-bromophenoxy)methyl)-5-cyclopropyl-4-methylmorpholine. ¹H NMR (400 MHz, DMSO-d6) δ=8.62 (br s, 1H), 8.08 (s, 1H), 7.90 (br s, 1H), 7.17 (br d, J=8.6 Hz, 2H, 2H), 6.91 (d, J=8.6 Hz, 2H), 4.05 (m, 1H), 3.82 (m, 2H), 3.79 (m, 1H), 3.17 (m, 3H), 2.37 (s, 3H), 1.24 (m, 2H), 0.53 (m, 1H), 0.33 (m, 2H), 0.15 (m, 1H), 0.00 (m, 1H). LCMS: Rt=0.74 min, m/z=452.4 (M+H). Chiral HPLC: Rt=1.54 min, de value=100%.

(3S,5S)-3-((4-bromophenoxy)methyl)-5-cyclopropyl-4-methylmorpholine was obtained using the procedure described for the synthesis of 6-((4-bromophenoxy)methyl)-5-methyl-2,8-dioxa-5-azaspiro[3.5]nonane except 6-((4-bromophenoxy)methyl)-2,8-dioxa-5-azaspiro[3.5]nonane was replaced with (3S,5S)-3-((4-bromophenoxy)methyl)-5-cyclopropylmorpholine.

(3S,5S)-3-((4-bromophenoxy)methyl)-5-cyclopropylmorpholine was obtained using the procedure described for the synthesis of 6-((4-bromophenoxy)methyl)-2,8-dioxa-5-azaspiro[3.5]nonane except (3-aminooxetan-3-yl)methanol was replaced with (S)-2-amino-2-cyclopropylethan-1-ol.

### Example 210: 5-(4-(((3R,5S)-5-cyclopropyl-4-methylmorpholin-3-yl)methoxy)phenyl)-2-oxo-6-(trifluoromethyl)-1,2-dihydropyridine-3-carboxamide (210)

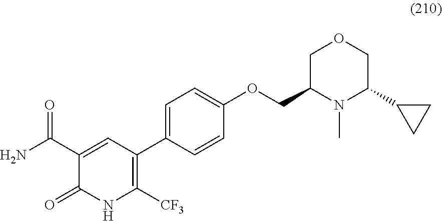 [SMILES: CN1[C@@H](COc2ccc(-c3cc(C(N)=O)c(=O)[nH]c3C(F)(F)F)cc2)COC[C@@H]1C1CC1]

5-(4-(((3R,5S)-5-cyclopropyl-4-methylmorpholin-3-yl)methoxy)phenyl)-2-oxo-6-(trifluoromethyl)-1,2-dihydropyridine-3-carboxamide (210) was obtained by SFC of the mixture containing Example 209. ¹H NMR (400 MHz, DMSO-d6) δ=8.85 (br s, 1H), 8.17 (s, 1H), 7.95 (br s, 1H), 7.24 (d, J=8.4 Hz, 2H), 7.04 (d, J=8.4 Hz, 2H), 4.21 (m, 2H), 3.71 (m, 1H), 3.67 (m, 4H), 3.03 (m, 1H), 2.57 (s, 3H), 1.82 (m, 1H), 0.59 (m, 2H), 0.57 (m, 1H), 0.28 (m, 1H), 0.05 (m, 1H). LCMS: Rt=0.72 min, m/z=452.4 (M+H). Chiral HPLC: Rt=1.74 min, de value=91.9%.

### Example 211: 5-(4-(((5S)-5-isopropyl-4-methylmorpholin-3-yl)methoxy)phenyl)-2-oxo-6-(trifluoromethyl)-1,2-dihydropyridine-3-carboxamide (211)

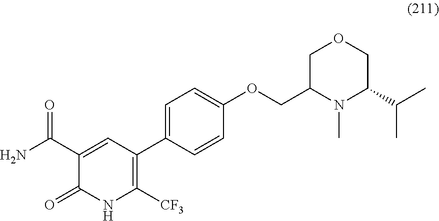 [SMILES: CC(C)[C@H]1COCC(COc2ccc(-c3cc(C(N)=O)c(=O)[nH]c3C(F)(F)F)cc2)N1C]

5-(4-(((5S)-5-isopropyl-4-methylmorpholin-3-yl)methoxy)phenyl)-2-oxo-6-(trifluoromethyl)-1,2-dihydropyridine-3-carboxamide (211) was made using steps 2 and 3 in the procedure used to make 2-oxo-5-(4-((tetrahydro-2H-pyran-4-ylmethylphenyl)-6-(trifluoromethyl)-1,2-dihydropyridine-3-carboxamide (52), except 4-(4-bromobenzylidene)tetrahydro-2H-pyran was replaced with (5S)-3-((4-bromophenoxy)methyl)-5-isopropyl-4-methylmorpholine. ¹H NMR: (400 MHz, DMSO-d6), δ=8.62 (br s, 2H), 8.24 (s, 1H), 8.09 (br s, 1H), 7.27 (m, 2H), 7.07 (m, 2H), 4.28 (m, 1H), 4.26 (m, 1H), 3.86 (m, 3H), 3.28 (m, 2H), 2.58 (m, 2H), 2.41 (s, 1.5H), 2.27 (s, 1.5H), 0.90 (m, 6H). LCMS: Rt=0.74 min, m/z=454.4 (M+H).

(5S)-3-((4-bromophenoxy)methyl)-5-isopropyl-4-methylmorpholine was obtained using the procedure described for the synthesis of 6-((4-bromophenoxy)methyl)-5-methyl-2,8-dioxa-5-azaspiro[3.5]nonane except 6-((4-bromophenoxy)methyl)-2,8-dioxa-5-azaspiro[3.5]nonane was replaced with (5S)-3-((4-bromophenoxy)methyl)-5-isopropylmorpholine.

(5S)-3-((4-bromophenoxy)methyl)-5-isopropylmorpholine was obtained using the procedure described for the synthesis of 6-((4-bromophenoxy)methyl)-2,8-dioxa-5-azaspiro[3.5]nonane except (3-aminooxetan-3-yl)methanol was replaced with ((S)-2-amino-3-methylbutan-1-ol.

### Example 212: 5-(4-((5-bromopyridin-3-yl)methoxy)phenyl)-2-oxo-6-(trifluoromethyl)-1,2-dihydropyridine-3-carboxamide (212)

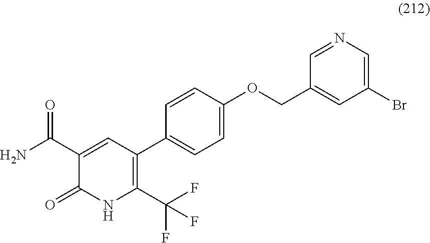 [SMILES: NC(=O)c1cc(-c2ccc(OCc3cncc(Br)c3)cc2)c(C(F)(F)F)[nH]c1=O]

5-(4-((5-bromopyridin-3-yl)methoxy)phenyl)-2-oxo-6-(trifluoromethyl)-1,2-dihydropyridine-3-carboxamide (212) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-bromo-5-(bromomethyl)pyridine. ¹H NMR: (400 MHz, DMSO-d6) δ: 10.61 (br. s, 1H), 8.69 (s, 2H), 8.13 (s, 1H), 7.82 (s, 1H), 7.15 (m, 2H), 7.02 (m, 2H), 5.17 (s, 2H); LCMS: Rt=0.90 min, m/z=470.1 [M+H].

### Example 213: 5-(4-((5-chloropyridin-3-yl)methoxy)phenyl)-2-oxo-6-(trifluoromethyl)-1,2-dihydropyridine-3-carboxamide (213)

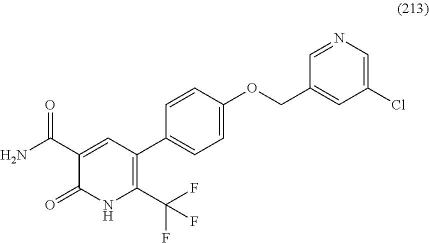 [SMILES: NC(=O)c1cc(-c2ccc(OCc3cncc(Cl)c3)cc2)c(C(F)(F)F)[nH]c1=O]

5-(4-((5-chloropyridin-3-yl)methoxy)phenyl)-2-oxo-6-(trifluoromethyl)-1,2-dihydropyridine-3-carboxamide (213) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)-5-chloropyridine. ¹H NMR: (400 MHz, DMSO-d6), δ 8.67 (d, J=1.6 Hz, 1H), 8.63 (d, J=2.0 Hz, 1H), 8.07 (m, 1H), 7.90 (s, 1H), 7.20 (d, J=8.8 Hz, 2H), 7.06 (d, J=8.8 Hz, 2H), 5.20 (s, 2H); LCMS: Rt=0.92 min, m/z=424.2 [M+H].

### Example 214: 2-oxo-5-(4-(pyridazin-3-ylmethoxy)phenyl)-6-(trifluoromethyl)-1,2-dihydropyridine-3-carboxamide (214)

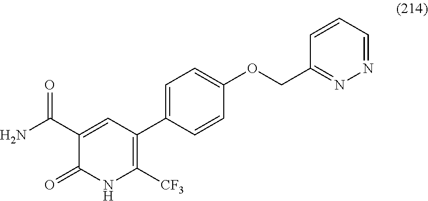 [SMILES: NC(=O)c1cc(-c2ccc(OCc3cccnn3)cc2)c(C(F)(F)F)[nH]c1=O]

2-oxo-5-(4-(pyridazin-3-ylmethoxy)phenyl)-6-(trifluoromethyl)-1,2-dihydropyridine-3-carboxamide (214) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)pyridazine. ¹H NMR (500 MHz, DMSO-d6) δ 13.65 (br. s, 1H), 9.24 (dd, J=4.9, 1.7 Hz, 1H), 8.46 (br. s, 1H), 8.28 (s, 1H), 8.18 (br. s, 1H), 7.87 (dd, J=8.4, 1.7 Hz, 1H), 7.78 (dd, J=8.5, 4.9 Hz, 1H), 7.34-7.25 (m, 2H), 7.21-7.11 (m, 2H), 5.47 (s, 2H). LCMS: Rt=0.61 min, m/z=391.2 [M+H].

### Example 215: 2-oxo-5-(4-(pyrimidin-4-ylmethoxy)phenyl)-6-(trifluoromethyl)-1,2-dihydropyridine-3-carboxamide (215)

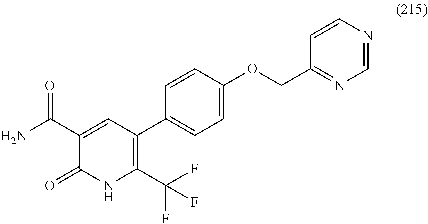 [SMILES: NC(=O)c1cc(-c2ccc(OCc3ccncn3)cc2)c(C(F)(F)F)[nH]c1=O]

2-oxo-5-(4-(pyrimidin-4-ylmethoxy)phenyl)-6-(trifluoromethyl)-1,2-dihydropyridine-3-carboxamide (215) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 4-(bromomethyl)pyrimidine. ¹H NMR (400 MHz, DMSO-d6), δ: 13.65 (br. s, 1H), 9.21 (s, 1H), 8.89 (d, J=4.4 Hz, 1H), 8.47 (s, 1H), 8.29 (s, 1H), 8.17 (s, 1H), 7.67 (d, J=4.4 Hz, 1H), 7.32 (d, J=8.4 Hz, 2H), 7.15 (d, J=8.8 Hz, 2H), 5.29 (s, 2H); LCMS: Rt=0.67 min, m/z=391.1 [M+H].

### Example 216: 5-(4-((6-chloropyrazin-2-yl)methoxy)phenyl)-2-oxo-6-(trifluoromethyl)-1,2-dihydropyridine-3-carboxamide (216)

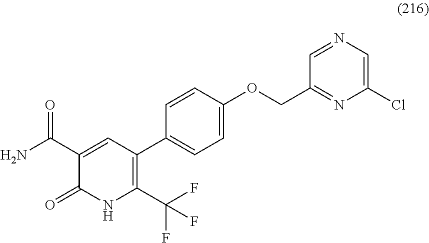 [SMILES: NC(=O)c1cc(-c2ccc(OCc3cncc(Cl)n3)cc2)c(C(F)(F)F)[nH]c1=O]

5-(4-((6-chloropyrazin-2-yl)methoxy)phenyl)-2-oxo-6-(trifluoromethyl)-1,2-dihydropyridine-3-carboxamide (216) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 2-(bromomethyl)-6-chloropyrazine. ¹H NMR (400 MHz, DMSO-d6) δ 9.82 (s, 1H), 8.84 (s, 1H), 8.81 (s, 1H), 7.99 (s, 1H), 7.47 (s, 1H), 7.23 (d, J=7.6 Hz, 2H), 7.11 (d, J=7.6 Hz, 2H), 5.29 (s, 2H); LCMS: Rt=0.74 min, m/z=425.0 [M+H].

### Example 217: 5-(4-((1-ethyl-1H-pyrazol-5-yl)methoxy)phenyl)-2-oxo-6-(trifluoromethyl)-1,2-dihydropyridine-3-carboxamide (217)

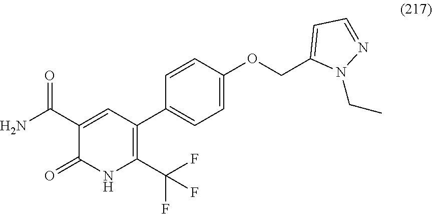 [SMILES: CCn1nccc1COc1ccc(-c2cc(C(N)=O)c(=O)[nH]c2C(F)(F)F)cc1]

5-(4-((1-ethyl-1H-pyrazol-5-yl)methoxy)phenyl)-2-oxo-6-(trifluoromethyl)-1,2-dihydropyridine-3-carboxamide (217) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 5-(bromomethyl)-1-ethyl-1H-pyrazole. ¹H NMR (400 MHz, MeOD-d4) δ 8.30 (s, 1H), 7.47 (d, J=2.0 Hz, 1H), 7.30 (d, J=8.4 Hz, 2H), 7.12 (d, J=8.8 Hz, 2H), 6.42 (d, J=1.6 Hz, 1H), 5.21 (s, 2H), 4.28 (m, 2H), 1.47 (m, 3H); ¹⁹FNMR: (377 MHz, MeOD-d4) δ −62.75 (br.s, 3F); LCMS: Rt=0.74 min, m/z=407.0 [M+H].

### Example 218: 5-(4-((1-(2-methoxyethyl)-1H-pyrazol-4-yl)methoxy)phenyl)-2-oxo-6-(trifluoromethyl)-1,2-dihydropyridine-3-carboxamide (218)

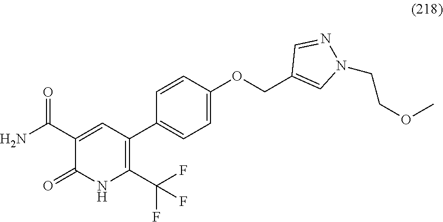 [SMILES: COCCn1cc(COc2ccc(-c3cc(C(N)=O)c(=O)[nH]c3C(F)(F)F)cc2)cn1]

5-(4-((1-(2-methoxyethyl)-1H-pyrazol-4-yl)methoxy)phenyl)-2-oxo-6-(trifluoromethyl)-1,2-dihydropyridine-3-carboxamide (218) was made using a procedure similar to that used to make 5-(4-((3-nitrobenzyl)oxy)phenyl)-2-oxo-6-(trifluoromethyl)-1,2-dihydropyridine-3-carboxamide (54), except 1-(bromomethyl)-3-nitrobenzene was replaced with 4-(bromomethyl)-1-(2-methoxyethyl)-1H-pyrazole. ¹H NMR (400 MHz, CDCl₃) δ 9.08 (br. s, 1H), 8.58 (s, 1H), 7.60 (d, J=6.8 Hz, 2H), 7.24 (d, J=8.4 Hz, 2H), 7.02 (d, J=8.8 Hz, 2H), 5.95-5.94 (m, 1H), 5.00 (s, 2H), 4.32-4.29 (m, 2H), 3.78-3.75 (m, 2H), 3.35 (s, 3H); ¹⁹F NMR (376 MHz, CHLOROFORM-d) δ −61.02 (s, 1F), −60.97-61.10 (m, 1F); LCMS: Rt=0.81 min, m/z=437.5 [M+H].

### Example 219: 5-(4-((1-(2-methoxyethyl)-1H-pyrazol-5-yl)methoxy)phenyl)-2-oxo-6-(trifluoromethyl)-1,2-dihydropyridine-3-carboxamide (219)

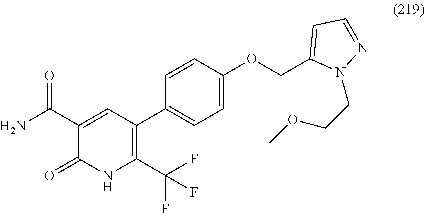 [SMILES: COCCn1nccc1COc1ccc(-c2cc(C(N)=O)c(=O)[nH]c2C(F)(F)F)cc1]

5-(4-((1-(2-methoxyethyl)-1H-pyrazol-5-yl)methoxy)phenyl)-2-oxo-6-(trifluoromethyl)-1,2-dihydropyridine-3-carboxamide (219) was made using a procedure similar to that used to make 5-(4-((3-nitrobenzyloxy)phenyl)-2-oxo-6-(trifluoromethyl)-1,2-dihydropyridine-3-carboxamide (54), except 1-(bromomethyl)-3-nitrobenzene was replaced with 5-(bromomethyl)-1-(2-methoxyethyl)-1H-pyrazole. ¹H NMR: (400 MHz, Chloroform-d3) δ 9.06 (d, J=1.2 Hz, 1H), 8.54 (s, 1H), 7.52 (d, J=1.6 Hz, 1H), 7.25 (d, J=8.8 Hz, 2H), 7.04 (d, J=8.4 Hz, 2H), 6.34 (d, J=2.0 Hz, 1H), 5.91 (d, J=2.8 Hz, 1H), 5.16 (s, 2H), 4.40 (m, 2H), 3.80 (m, 2H), 3.31 (s, 3H); ¹⁹F NMR: (377 MHz, Chloroform-d3) δ 61.11 (s, 1F), −60.84-61.46 (m, 1F); LCMS: Rt=0.82 min, m/z=437 [M+H]

### Example 220: 5-(4-((1-methyl-1H-pyrazol-4-yl)methoxy)phenyl)-2-oxo-6-(trifluoromethyl)-1,2-dihydropyridine-3-carboxamide (220)

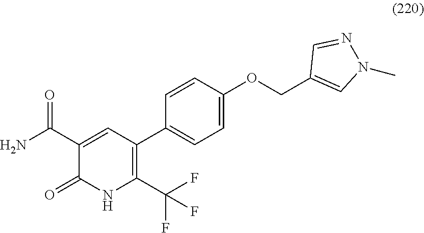 [SMILES: Cn1cc(COc2ccc(-c3cc(C(N)=O)c(=O)[nH]c3C(F)(F)F)cc2)cn1]

5-(4-((1-methyl-1H-pyrazol-4-yl)methoxy)phenyl)-2-oxo-6-(trifluoromethyl)-1,2-dihydropyridine-3-carboxamide (220) was made using a procedure similar to that used to make 5-(4-((3-nitrobenzyloxy)phenyl)-2-oxo-6-(trifluoromethyl)-1,2-dihydropyridine-3-carboxamide (54), except 1-(bromomethyl)-3-nitrobenzene was replaced with 4-(bromomethyl)-1-methyl-1H-pyrazole. ¹H NMR (400 MHz, DMSO-d6) δ 8.14 (s, 1H), 7.81 (s, 1H), 7.51 (s, 1H), 7.22 (d, J=8.3 Hz, 2H), 7.03 (d, J=8.5 Hz, 2H), 4.98 (s, 2H), 3.83 (s, 3H); ¹⁹F NMR (376 MHz, DMSO-d6) δ −60.36 (s, 3F); LCMS: Rt=0.65 min, m/z=393.0 [M+H].

### Example 221: 5-(4-((1-(cyclopropylmethyl)-1H-pyrazol-4-yl)methoxy)phenyl)-2-oxo-6-(trifluoromethyl)-1,2-dihydropyridine-3-carboxamide (221)

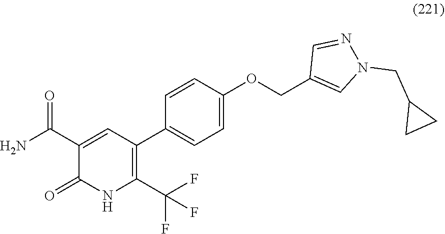 [SMILES: NC(=O)c1cc(-c2ccc(OCc3cnn(CC4CC4)c3)cc2)c(C(F)(F)F)[nH]c1=O]

5-(4-((1-(cyclopropylmethyl)-1H-pyrazol-4-yl)methoxy)phenyl)-2-oxo-6-(trifluoromethyl)-1,2-dihydropyridine-3-carboxamide (221) was made using a procedure similar to that used to make 5-(4-((3-nitrobenzyloxy)phenyl)-2-oxo-6-(trifluoromethyl)-1,2-dihydropyridine-3-carboxamide (54), except 1-(bromomethyl)-3-nitrobenzene was replaced with 4-(bromomethyl)-1-(cyclopropylmethyl)-1H-pyrazole. ¹H NMR (400 MHz, DMSO-d6) δ 8.14 (s, 1H), 7.90 (s, 1H), 7.53 (s, 1H), 7.24 (d, J=8.6 Hz, 2H), 7.06 (d, J=8.6 Hz, 2H), 5.00 (s, 2H), 3.96 (d, J=7.2 Hz, 2H), 2.09 (s, 1H), 1.28-1.17 (m, 1H), 0.57-0.48 (m, 2H), 0.40-0.32 (m, 2H); ¹⁹F NMR (376 MHz, DMSO-d6) δ −40.38 (s, 3F); LCMS: Rt=0.85 min, m/z=433.1 [M+H].

### Example 222: 5-(4-((1-ethyl-1H-pyrazol-4-yl)methoxy)phenyl)-2-oxo-6-(trifluoromethyl)-1,2-dihydropyridine-3-carboxamide (222)

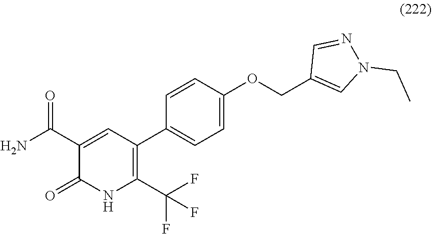 [SMILES: CCn1cc(COc2ccc(-c3cc(C(N)=O)c(=O)[nH]c3C(F)(F)F)cc2)cn1]

5-(4-((1-ethyl-1H-pyrazol-4-yl)methoxy)phenyl)-2-oxo-6-(trifluoromethyl)-1,2-dihydropyridine-3-carboxamide (222) was made using a procedure similar to that used to make 5-(4-((3-nitrobenzyloxy)phenyl)-2-oxo-6-(trifluoromethyl)-1,2-dihydropyridine-3-carboxamide (54), except 1-(bromomethyl)-3-nitrobenzene was replaced with 4-(bromomethyl)-1-ethyl-1H-pyrazole. ¹H NMR (400 MHz, methanol-d4) δ 8.34-8.28 (m, 1H), 7.76 (s, 1H), 7.57 (s, 1H), 7.25 (d, J=8.5 Hz, 2H), 7.05 (d, J=8.7 Hz, 2H), 5.04 (s, 2H), 4.19 (q, J=7.3 Hz, 2H), 1.45 (t, J=7.3 Hz, 3H); ¹⁹F NMR (376 MHz, methanol-d4) δ −62.75 (br.s, 3F); LCMS: Rt=0.83 min, m/z=407.1 [M+H].

### Example 223: 5-(4-(isoxazol-3-ylmethoxy)phenyl)-2-oxo-6-(trifluoromethyl)-1,2-dihydropyridine-3-carboxamide (223)

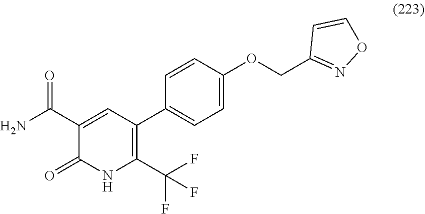 [SMILES: NC(=O)c1cc(-c2ccc(OCc3ccon3)cc2)c(C(F)(F)F)[nH]c1=O]

5-(4-(isoxazol-3-ylmethoxy)phenyl)-2-oxo-6-(trifluoromethyl)-1,2-dihydropyridine-3-carboxamide (223) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 3-(bromomethyl)isoxazole. ¹H NMR: (400 MHz, DMSO-d6), δ: 8.97 (d, J=1.0 Hz, 2H), 8.18 (d, J=7.1 Hz, 1H), 7.95 (s, 1H), 7.27 (d, J=8.4 Hz, 2H), 7.11 (d, J=8.7 Hz, 2H), 6.74 (d, J=1.3 Hz, 1H), 5.29 (s, 2H); LCMS: Rt=0.68 min, m/z=380.0 [M+H].

### Example 224: 5-(4-((5-methylisoxazol-3-yl)methoxy)phenyl)-2-oxo-6-(trifluoromethyl)-1,2-dihydropyridine-3-carboxamide (224)

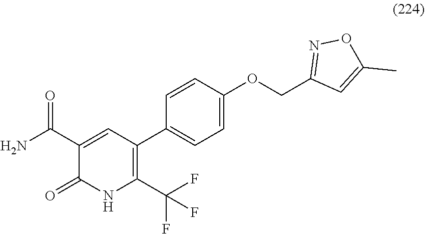 [SMILES: Cc1cc(COc2ccc(-c3cc(C(N)=O)c(=O)[nH]c3C(F)(F)F)cc2)no1]

5-(4-((5-methylisoxazol-3-yl)methoxy)phenyl)-2-oxo-6-(trifluoromethyl)-1,2-dihydropyridine-3-carboxamide (224) was made using a procedure similar to that used to make 5-(4-((3-nitrobenzyloxy)phenyl)-2-oxo-6-(trifluoromethyl)-1,2-dihydropyridine-3-carboxamide (54), except 1-(bromomethyl)-3-nitrobenzene was replaced with 3-(bromomethyl)-5-methylisoxazole. ¹H NMR (400 MHz, DMSO-d6) δ 8.17 (d, J=6.2 Hz, 1H), 7.94 (s, 1H), 7.27 (d, J=8.4 Hz, 2H), 7.09 (d, J=8.7 Hz, 2H), 6.37 (s, 1H), 5.19 (s, 2H), 2.43 (s, 3H); LCMS: Rt=0.71 min, m/z=394.0 [M+H].

### Example 225: 5-(4-(oxetan-2-ylmethoxy)phenyl)-2-oxo-6-(trifluoromethyl)-1,2-dihydropyridine-3-carboxamide (225)

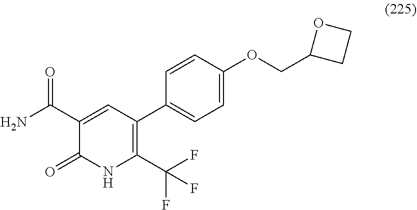 [SMILES: NC(=O)c1cc(-c2ccc(OCC3CCO3)cc2)c(C(F)(F)F)[nH]c1=O]

5-(4-(oxetan-2-ylmethoxy)phenyl)-2-oxo-6-(trifluoromethyl)-1,2-dihydropyridine-3-carboxamide (225) was made using a procedure similar to that used to make 5-(4-((1-acetylpiperidin-4-ylmethoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with oxetan-2-ylmethanol. ¹H NMR (400 MHz, DMSO-d6) δ 8.15 (s, 1H), 8.00-7.71 (m, 1H), 7.23 (d, J=8.0 Hz, 2H), 7.03 (d, J=8.0 Hz, 2H), 5.03 (m, 1H), 4.61-4.45 (m, 2H), 4.23-4.17 (m, 1H), 4.16-4.11 (m, 1H), 2.76-2.64 (m, 2H); LCMS: Rt=0.66 min, m/z=369.1 [M+H].

### Example 226: 5-(4-((3-fluorooxetan-3-yl)methoxy)phenyl)-2-oxo-6-(trifluoromethyl)-1,2-dihydropyridine-3-carboxamide (226)

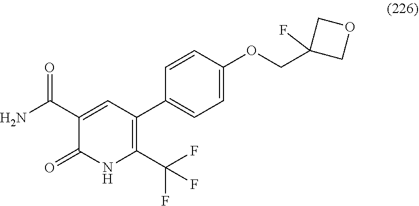 [SMILES: NC(=O)c1cc(-c2ccc(OCC3(F)COC3)cc2)c(C(F)(F)F)[nH]c1=O]

5-(4-((3-fluorooxetan-3-yl)methoxy)phenyl)-2-oxo-6-(trifluoromethyl)-1,2-dihydropyridine-3-carboxamide (226) was made using a procedure similar to that used to make 5-(4-((1-acetylpiperidin-4-ylmethoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (3-fluorooxetan-3-yl)methanol. ¹H NMR (500 MHz, DMSO-d6) δ 13.66 (br. s, 1H), 8.52 (br. s, 1H), 8.26 (s, 1H), 8.13 (br. s, 1H), 7.30 (d, J=8.4 Hz, 2H), 7.08 (d, J=8.6 Hz, 2H), 4.79-4.67 (m, 4H), 4.48 (d, J=22.1 Hz, 2H). LCMS: Rt=0.69 min, m/z=387.2 [M+H].

### Example 227: 5-(4-((3-methyl)oxetan-3-yl)methoxy)phenyl)-2-oxo-6-(trifluoromethyl)-1,2-dihydropyridine-3-carboxamide (227)

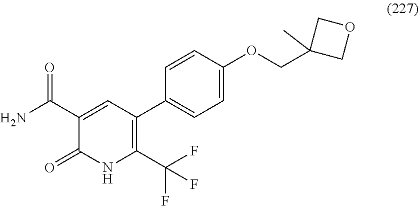 [SMILES: CC1(COc2ccc(-c3cc(C(N)=O)c(=O)[nH]c3C(F)(F)F)cc2)COC1]

5-(4-((3-methyl)oxetan-3-yl)methoxy)phenyl)-2-oxo-6-(trifluoromethyl)-1,2-dihydropyridine-3-carboxamide (227) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (3-methyl)oxetan-3-yl)methanol. ¹H NMR (500 MHz, DMSO-d6) δ 13.62 (br. s, 1H), 8.46 (br. s, 1H), 8.27 (s, 1H), 8.17 (br. s, 1H), 7.34-7.21 (m, 2H), 7.09-7.01 (m, 2H), 4.51 (d, J=5.8 Hz, 2H), 4.32 (d, J=5.8 Hz, 2H), 4.10 (s, 2H), 1.38 (s, 3H). LCMS: Rt=0.77 min, m/z=383.2 [M+H].

### Example 228: 5-(4-((3-ethyl)oxetan-3-yl)methoxy)phenyl)-2-oxo-6-(trifluoromethyl)-1,2-dihydropyridine-3-carboxamide (228)

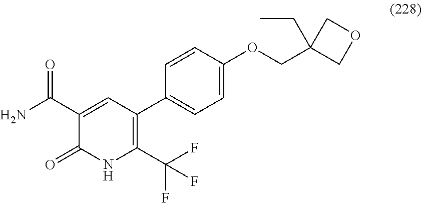 [SMILES: CCC1(COc2ccc(-c3cc(C(N)=O)c(=O)[nH]c3C(F)(F)F)cc2)COC1]

5-(4-((3-ethyl)oxetan-3-yl)methoxy)phenyl)-2-oxo-6-(trifluoromethyl)-1,2-dihydropyridine-3-carboxamide (228) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (3-ethyl)oxetan-3-yl)methanol. ¹H NMR (400 MHz, DMSO-d6) δ 8.22 (s, 1H), 8.15 (s, 1H), 7.27 (d, J=8.6 Hz, 2H), 7.07 (d, J=8.6 Hz, 2H), 4.47 (d, J=6.0 Hz, 2H), 4.36 (d, J=6.0 Hz, 2H), 4.15 (s, 2H), 1.81 (q, J=7.4 Hz, 2H), 0.92 (t, J=7.4 Hz, 3H). LCMS: Rt=0.72 min, m/z=397.1 [M+H].

### Example 229: 5-(4-((3-(cyanomethyl)oxetan-3-yl)methoxy)phenyl)-2-oxo-6-(trifluoromethyl)-1,2-dihydropyridine-3-carboxamide (229)

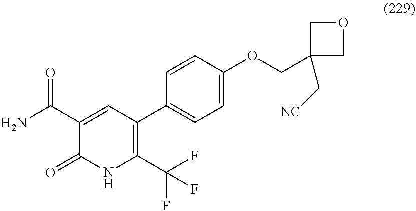 [SMILES: N#CCC1(COc2ccc(-c3cc(C(N)=O)c(=O)[nH]c3C(F)(F)F)cc2)COC1]

5-(4-((3-(cyanomethyl)oxetan-3-yl)methoxy)phenyl)-2-oxo-6-(trifluoromethyl)-1,2-dihydropyridine-3-carboxamide (229) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 2-(3-(hydroxymethyl)oxetan-3-yl)acetonitrile. ¹H NMR (400 MHz, DMSO-d6) δ 8.97 (br. s, 1H), 8.17 (br. s, 1H), 7.90 (br. s, 1H), 7.27 (br.d, J=8.4 Hz, 2H), 7.07 (d, J=8.4 Hz, 2H), 4.56-4.50 (m, 4H), 4.27 (s, 2H), 3.09 (s, 2H); LCMS: Rt=0.65 min, m/z=408.1 [M+H].

### Example 230: 5-(4-((2-oxaspiro[3.3]heptan-6-yl)oxy)phenyl)-2-oxo-6-(trifluoromethyl)-1,2-dihydropyridine-3-carboxamide (230)

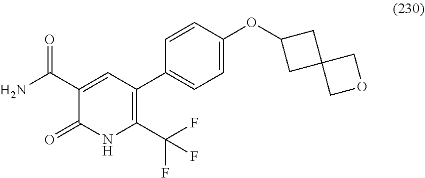 [SMILES: NC(=O)c1cc(-c2ccc(OC3CC4(COC4)C3)cc2)c(C(F)(F)F)[nH]c1=O]

5-(4-((2-oxaspiro[3.3]heptan-6-yl)oxy)phenyl)-2-oxo-6-(trifluoromethyl)-1,2-dihydropyridine-3-carboxamide (230) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 2-oxaspiro[3.3]heptan-6-ol. LCMS: Rt=0.75 min, m/z=395.3 [M+H].

### Example 231: 2-oxo-5-(4-((tetrahydrofuran-3-yl)methoxy)phenyl)-6-(trifluoromethyl)-1,2-dihydropyridine-3-carboxamide (231)

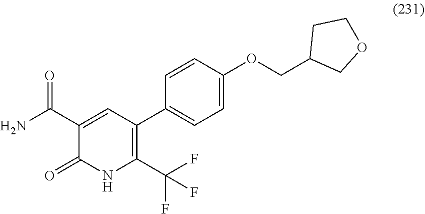 [SMILES: NC(=O)c1cc(-c2ccc(OCC3CCOC3)cc2)c(C(F)(F)F)[nH]c1=O]

2-oxo-5-(4-((tetrahydrofuran-3-yl)methoxy)phenyl)-6-(trifluoromethyl)-1,2-dihydropyridine-3-carboxamide (231) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (tetrahydrofuran-3-yl)methanol. ¹H NMR (400 MHz, DMSO-d6) δ 8.33 (s, 2H), 7.81 (s, 1H), 7.13 (d, J=8.8 Hz, 2H), 7.05 (m, 1H), 6.94 (d, J=8.8 Hz, 2H), 3.96 (m, 2H), 3.81-3.79 (m, 3H), 3.67 (m, 2H), 3.54 (m, 1H), 2.07 (m, 1H), 1.70 (m, 1H); LCMS: Rt=0.70 min, m/z=383.1 [M+H].

### Example 232: 2-oxo-5-(4-((tetrahydrofuran-2-yl)methoxy)phenyl)-6-(trifluoromethyl)-1,2-dihydropyridine-3-carboxamide (232)

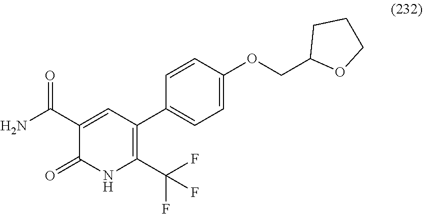 [SMILES: NC(=O)c1cc(-c2ccc(OCC3CCCO3)cc2)c(C(F)(F)F)[nH]c1=O]

2-oxo-5-(4-((tetrahydrofuran-2-yl)methoxy)phenyl)-6-(trifluoromethyl)-1,2-dihydropyridine-3-carboxamide (232) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (tetrahydrofuran-2-yl)methanol. ¹H NMR (500 MHz, DMSO-d6) δ 13.62 (br. s, 1H), 8.52 (br. s, 1H), 8.26 (s, 1H), 8.13 (br. s, 1H), 7.25 (d, J=8.3 Hz, 2H), 7.12-6.91 (m, 2H), 4.17 (qd, J=6.8, 3.9 Hz, 1H), 4.01 (dd, J=10.2, 4.0 Hz, 1H), 3.96 (dd, J=10.1, 6.3 Hz, 1H), 3.83-3.76 (m, 1H), 3.69 (td, J=7.7, 6.2 Hz, 1H), 2.01 (dtd, J=12.4, 7.7, 5.4 Hz, 1H), 1.95-1.78 (m, 2H), 1.74-1.61 (m, 1H). LCMS: Rt=0.78 min, m/z=383.3 [M+H].

### Example 233: 5-(4-((3-methyl-2-oxooxazolidin-5-yl)methoxy)phenyl)-2-oxo-6-(trifluoromethyl)-1,2-dihydropyridine-3-carboxamide (233)

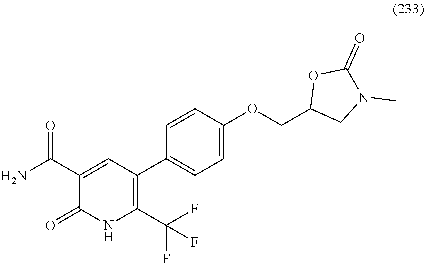 [SMILES: CN1CC(COc2ccc(-c3cc(C(N)=O)c(=O)[nH]c3C(F)(F)F)cc2)OC1=O]

5-(4-((3-methyl-2-oxooxazolidin-5-yl)methoxy)phenyl)-2-oxo-6-(trifluoromethyl)-1,2-dihydropyridine-3-carboxamide (233) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 5-(hydroxymethyl)-3-methyloxazolidin-2-one. ¹H NMR (400 MHz, DMSO-d6) δ 10.01 (br. s, 1H), 8.20 (s, 1H), 7.94 (s, 1H), 7.37 (br. s, 1H), 7.19 (d, J=8.4 Hz, 2H), 6.99 (d, J=8.4 Hz, 2H), 4.87 (m, 1H), 4.20 (m, 2H), 3.72 (m, 1H), 2.79 (s, 3H); LCMS: Rt=0.68 min, m/z=412.1 [M+H].

### Example 234: 5-(4-((3-ethyl-2-oxooxazolidin-5-yl)methoxy)phenyl)-2-oxo-6-(trifluoromethyl)-1,2-dihydropyridine-3-carboxamide (234)

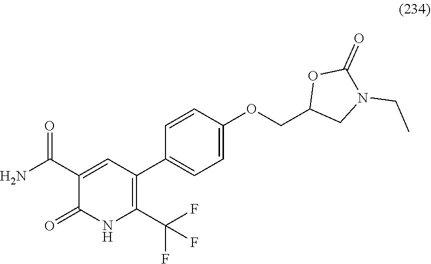 [SMILES: CCN1CC(COc2ccc(-c3cc(C(N)=O)c(=O)[nH]c3C(F)(F)F)cc2)OC1=O]

5-(4-((3-ethyl-2-oxooxazolidin-5-yl)methoxy)phenyl)-2-oxo-6-(trifluoromethyl)-1,2-dihydropyridine-3-carboxamide (234) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 3-ethyl-5-(hydroxymethyl)oxazolidin-2-one. ¹H NMR (400 MHz, DMSO-d6) δ 8.15 (s, 1H), 8.04 (s, 1H), 7.21 (d, J=7.9 Hz, 2H), 7.00 (d, J=8.4 Hz, 2H), 4.92-4.83 (m, 1H), 4.26-4.10 (m, 2H), 3.71 (m, 1H), 3.42 (m, 1H), 3.22 (m, 2H), 1.09 (m, 3H); LCMS: Rt=0.70 min, MS=426.1 [M+H].

### Example 235: 5-(4-((3-methyl-2-oxooxazolidin-4-yl)methoxy)phenyl)-2-oxo-6-(trifluoromethyl)-1,2-dihydropyridine-3-carboxamide (235)

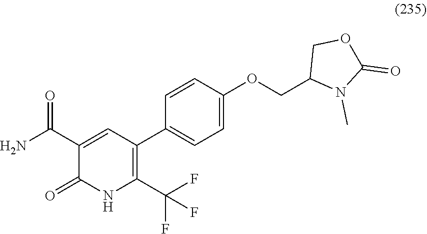 [SMILES: CN1C(=O)OCC1COc1ccc(-c2cc(C(N)=O)c(=O)[nH]c2C(F)(F)F)cc1]

5-(4-((3-methyl-2-oxooxazolidin-4-yl)methoxy)phenyl)-2-oxo-6-(trifluoromethyl)-1,2-dihydropyridine-3-carboxamide (235) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 4-(hydroxymethyl)-3-methyloxazolidin-2-one. ¹H NMR (400 MHz, DMSO-d6) δ 8.83 (br. s, 1H), 8.19 (m, 1H), 7.94 (br. s, 1H), 7.27 (d, J=8.4 Hz, 2H), 7.05 (d, J=8.4 Hz, 2H), 4.43 (m, 1H), 4.24 (m, 1H), 4.17 (m, 3H), 2.82 (s, 3H); LCMS: Rt=0.62 min, m/z=412.1 [M+H].

### Example 236: 2-oxo-5-(4-((tetrahydro-2H-pyran-2-yl)methoxy)phenyl)-6-(trifluoromethyl)-1,2-dihydropyridine-3-carboxamide (236)

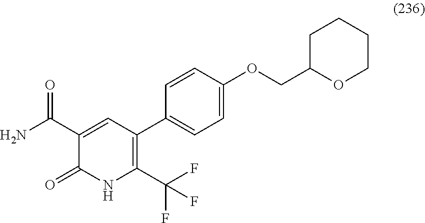 [SMILES: NC(=O)c1cc(-c2ccc(OCC3CCCCO3)cc2)c(C(F)(F)F)[nH]c1=O]

2-oxo-5-(4-((tetrahydro-2H-pyran-2-yl)methoxy)phenyl)-6-(trifluoromethyl)-1,2-dihydropyridine-3-carboxamide (236) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (tetrahydro-2H-pyran-2-yl)methanol. ¹H NMR (500 MHz, DMSO-d6) δ 13.63 (br. s, 1H), 8.46 (br. s, 1H), 8.28 (s, 1H), 8.17 (br. s, 1H), 7.32-7.22 (m, 3H), 7.08-6.99 (m, 3H), 3.99-3.93 (m, 3H), 3.93-3.87 (m, 1H), 3.68-3.60 (m, 1H), 3.40 (dd, J=11.4, 3.6 Hz, 3H), 1.83 (d, J=12.5 Hz, 1H), 1.71-1.60 (m, 1H), 1.58-1.43 (m, 3H), 1.34 (qd, J=12.4, 4.0 Hz, 1H). LCMS: Rt=0.88 min, m/z=397.3 [M+H].

### Example 237 and Example 238: 5-(4-((1,4-dioxan-2-yl)methoxy)phenyl)-2-oxo-6-(trifluoromethyl)-1,2-dihydropyridine-3-carboxamide (SFC Peak 1) (237) and Example 238: 5-(4-((1,4-dioxan-2-yl)methoxy)phenyl)-2-oxo-6-(trifluoromethyl)-1,2-dihydropyridine-3-carboxamide (SFC Peak 2) (238)

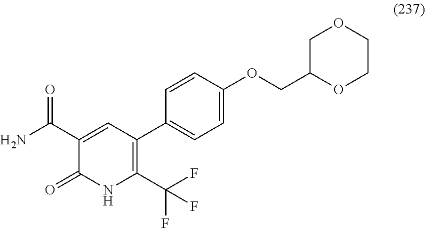 [SMILES: NC(=O)c1cc(-c2ccc(OCC3COCCO3)cc2)c(C(F)(F)F)[nH]c1=O]

Rac-5-(4-((1,4-dioxan-2-yl)methoxy)phenyl)-2-oxo-6-(trifluoromethyl)-1,2-dihydropyridine-3-carboxamide was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (1,4-dioxan-2-yl)methanol.

Two Peaks were Obtained by SFC Separation:

SFC peak 1: 5-(4-((1,4-dioxan-2-yl)methoxy)phenyl)-2-oxo-6-(trifluoromethyl)-1,2-dihydropyridine-3-carboxamide (237): ¹H NMR (400 MHz, DMSO-d6) δ 13.65 (s, 1H), 8.54 (s, 1H), 8.26 (s, 1H), 8.14 (s, 1H), 7.28 (d, J=8.0 Hz, 2H), 7.04 (d, J=8.0 Hz, 2H), 4.01-4.00 (m, 2H), 3.86-3.67 (m, 3H), 3.67-3.42 (m, 2H), 3.34-2.53 (m, 1H), 2.52-2.50 (m, 2H); LCMS: Rt=0.69 min, m/z=399.1 [M+H]; SFC: Rt=2.73 min (peak 1), e.e. value 100%.

SFC peak 2: 5-(4-((1,4-dioxan-2-yl)methoxy)phenyl)-2-oxo-6-(trifluoromethyl)-1,2-dihydropyridine-3-carboxamide (238): separation. ¹H NMR (400 MHz, DMSO-d6) δ 13.65 (br. s, 1H), 8.49 (br. s, 1H), 8.27 (s, 1H), 8.17 (br. s, 1H), 7.28 (d, J=8.0 Hz, 2H), 7.04 (d, J=8.0 Hz, 2H), 4.02-4.00 (m, 2H), 3.86-3.83 (m, 3H), 3.76-3.67 (m, 2H), 3.67-3.65 (m, 1H), 3.42-3.39 (m, 2H); LCMS: Rt=0.69 min, m/z=399.1 [M+H]; SFC: Rt=3.06 min (peak 2), e.e. value 100%.

### Example 239: 5-(4-((1,4-dioxan-2-yl)methoxy)-3-fluorophenyl)-2-oxo-6-(trifluoromethyl)-1,2-dihydropyridine-3-carboxamide (239)

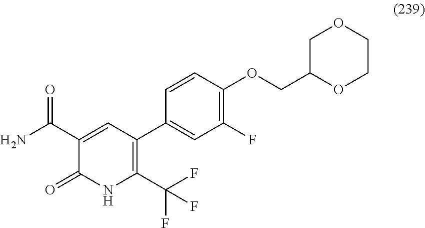 [SMILES: NC(=O)c1cc(-c2ccc(OCC3COCCO3)c(F)c2)c(C(F)(F)F)[nH]c1=O]

5-(4-((1,4-dioxan-2-yl)methoxy)-3-fluorophenyl)-2-oxo-6-(trifluoromethyl)-1,2-dihydropyridine-3-carboxamide (239) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 4-(4,4,5,5-tetramethyl-1,3,2-dioxaborolan-2-yl)phenol was replaced with 2-fluoro-4-(4,4,5,5-tetramethyl-1,3,2-dioxaborolan-2-yl)phenol, and 1-(4-(hydroxymethyl)piperidin-1-yl)ethan-1-one was replaced with (1,4-dioxan-2-yl)methanol. ¹H NMR (400 MHz, DMSO-d6) δ 8.63 (s, 1H), 8.25 (s, 1H), 8.14-8.12 (m, 1H), 7.28-7.25 (m, 2H), 7.11 (d, J=8.8 Hz, 1H), 4.10 (d, J=4.8 Hz, 2H), 3.86-3.83 (m, 1H), 3.76-3.67 (m, 1H), 3.66-3.65 (m, 1H), 3.65-3.51 (m, 2H), 3.51-3.50 (m, 1H), 3.50-3.45 (m, 1H); LCMS: Rt=0.67 min, m/z=417.1 [M+H].

### Example 240: 5-(4-((1,4-dioxan-2-yl)methoxy)-3-chlorophenyl)-2-oxo-6-(trifluoromethyl)-1,2-dihydropyridine-3-carboxamide (240)

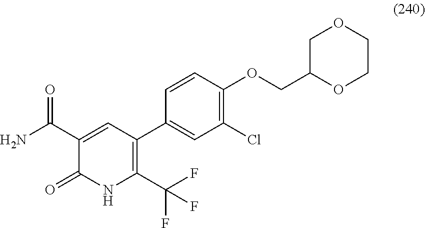 [SMILES: NC(=O)c1cc(-c2ccc(OCC3COCCO3)c(Cl)c2)c(C(F)(F)F)[nH]c1=O]

5-(4-((1,4-dioxan-2-yl)methoxy)-3-chlorophenyl)-2-oxo-6-(trifluoromethyl)-1,2-dihydropyridine-3-carboxamide (240) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 4-(4,4,5,5-tetramethyl-1,3,2-dioxaborolan-2-yl)phenol was replaced with 2-chloro-4-(4,4,5,5-tetramethyl-1,3,2-dioxaborolan-2-yl)phenol, and 1-(4-(hydroxymethyl)piperidin-1-yl)ethan-1-one was replaced with (1,4-dioxan-2-yl)methanol. ¹H NMR (400 MHz, DMSO-d6) δ 8.17 (br. s, 1H), 8.07 (br. s, 1H), 7.69-7.62 (m, 1H), 7.35 (s, 1H), 7.20 (s, 2H), 4.12-4.10 (m, 2H), 4.09-3.89 (m, 2H), 3.89-3.77 (m, 1H), 3.68-3.67 (m, 2H), 3.50-3.48 (m, 2H); LCMS: Rt=0.70 min, m/z=433.0 [M+H].

### Example 241: 5-(4-((1,4-dioxan-2-yl)methoxy)-3-cyanophenyl)-2-oxo-6-(trifluoromethyl)-1,2-dihydropyridine-3-carboxamide (241)

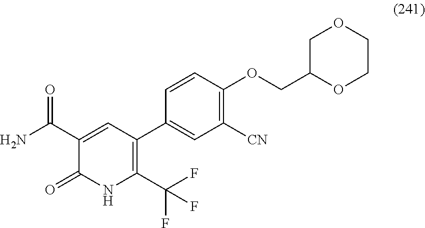 [SMILES: N#Cc1cc(-c2cc(C(N)=O)c(=O)[nH]c2C(F)(F)F)ccc1OCC1COCCO1]

5-(4-((1,4-dioxan-2-yl)methoxy)-3-cyanophenyl)-2-oxo-6-(trifluoromethyl)-1,2-dihydropyridine-3-carboxamide (241) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 4-(4,4,5,5-tetramethyl-1,3,2-dioxaborolan-2-yl)phenol was replaced with 2-hydroxy-5-(4,4,5,5-tetramethyl-1,3,2-dioxaborolan-2-yl)benzonitrile, and 1-(4-(hydroxymethyl)piperidin-1-yl)ethan-1-one was replaced with (1,4-dioxan-2-yl)methanol. ¹H NMR (400 MHz, DMSO-d6) δ 13.73 (br.s, 1H), 8.50 (br.s, 1H), 8.29 (s, 1H), 8.17-7.78 (m, 1H), 7.77 (s, 1H), 7.65-7.63 (m, 1H), 7.38 (d, J=8.8 Hz, 1H), 4.25-4.23 (m, 2H), 4.23-4.22 (m, 1H), 3.89-3.86 (m, 2H), 3.78-3.69 (m, 2H), 3.51-3.48 (m, 2H); LCMS: Rt=0.65 min, m/z=424.0 [M+H].

### Example 242: 5-(4-((1,4-dioxan-2-yl)methoxy)-3-methylphenyl)-2-oxo-6-(trifluoromethyl)-1,2-dihydropyridine-3-carboxamide (242)

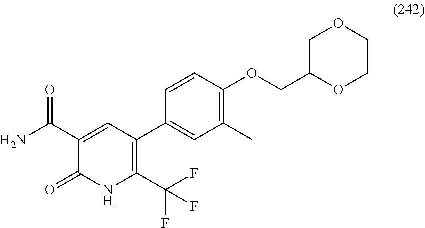 [SMILES: Cc1cc(-c2cc(C(N)=O)c(=O)[nH]c2C(F)(F)F)ccc1OCC1COCCO1]

5-(4-((1,4-dioxan-2-yl)methoxy)-3-methylphenyl)-2-oxo-6-(trifluoromethyl)-1,2-dihydropyridine-3-carboxamide (242) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 4-(4,4,5,5-tetramethyl-1,3,2-dioxaborolan-2-yl)phenol was replaced with 2-methyl-4-(4,4,5,5-tetramethyl-1,3,2-dioxaborolan-2-yl)phenol, and 1-(4-(hydroxymethyl)piperidin-1-yl)ethan-1-one was replaced with (1,4-dioxan-2-yl)methanol. ¹H NMR (400 MHz, DMSO-d6) δ 8.19 (s, 1H), 8.02 (br. s, 1H), 7.58 (br. s, 1H), 7.05 (m, 1H), 6.96 (d, J=8.2 Hz, 1H), 4.01 (m, 2H), 3.98 (m, 2H), 3.89 (m, 1H), 3.68 (m, 2H), 3.52 (m, 2H), 2.18 (s, 3H); LCMS: Rt=0.69 min, m/z=413.1 [M+H].

### Example 243: 5-(4-((1,4-dioxan-2-yl)methoxy)-3-cyclopropylphenyl)-2-oxo-6-(trifluoromethyl)-1,2-dihydropyridine-3-carboxamide (243)

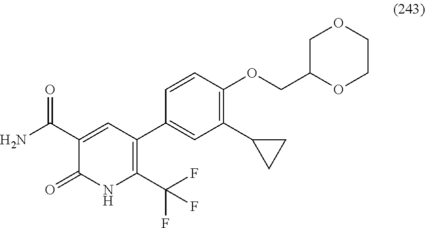 [SMILES: NC(=O)c1cc(-c2ccc(OCC3COCCO3)c(C3CC3)c2)c(C(F)(F)F)[nH]c1=O]

5-(4-((1,4-dioxan-2-yl)methoxy)-3-cyclopropylphenyl)-2-oxo-6-(trifluoromethyl)-1,2-dihydropyridine-3-carboxamide (243) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 4-(4,4,5,5-tetramethyl-1,3,2-dioxaborolan-2-yl)phenol was replaced with 2-cyclopropyl-4-(4,4,5,5-tetramethyl-1,3,2-dioxaborolan-2-yl)phenol, and 1-(4-(hydroxymethyl)piperidin-1-yl)ethan-1-one was replaced with (1,4-dioxan-2-yl)methanol. ¹H NMR (400 MHz, DMSO-d6) δ 8.16 (s, 1H), 7.95-8.09 (m, 1H), 7.60 (s, 1H), 6.93-7.04 (m, 2H), 6.70 (s, 1H), 3.86-4.10 (m, 4H), 3.77-3.82 (m, 1H), 3.63-3.75 (m, 2H), 3.42-3.50 (m, 3H), 2.34-2.37 (m, 1H), 2.09-2.20 (m, 1H), 0.86-0.95 (m, 2H), 0.58-0.67 (m, 2H). LCMS: Rt=0.73 min, m/z=439.1 [M+H].

### Example 244: 5-(4-((1,4-dioxan-2-yl)methoxy)-3-methoxyphenyl)-2-oxo-6-(trifluoromethyl)-1,2-dihydropyridine-3-carboxamide (244)

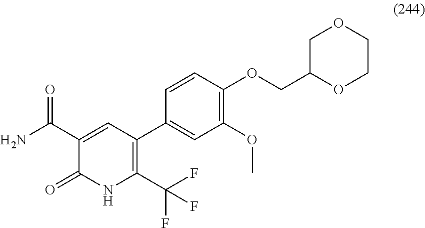 [SMILES: COc1cc(-c2cc(C(N)=O)c(=O)[nH]c2C(F)(F)F)ccc1OCC1COCCO1]

5-(4-((1,4-dioxan-2-yl)methoxy)-3-methoxyphenyl)-2-oxo-6-(trifluoromethyl)-1,2-dihydropyridine-3-carboxamide (244) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 4-(4,4,5,5-tetramethyl-1,3,2-dioxaborolan-2-yl)phenol was replaced with 2-methoxy-4-(4,4,5,5-tetramethyl-1,3,2-dioxaborolan-2-yl)phenol, and 1-(4-(hydroxymethyl)piperidin-1-yl)ethan-1-one was replaced with (1,4-dioxan-2-yl)methanol. ¹H NMR (400 MHz, DMSO-d6) δ 9.30 (br. s, 1H), 8.20 (br. s, 1H), 8.11 (br. s, 1H), 7.75 (br. s, 1H), 7.01 (d, J=8.0 Hz, 1H), 6.89 (br. s, 1H), 6.79 (d, J=7.6 Hz, 1H), 4.00-3.96 (m, 2H), 3.86 (d, J=12.0 Hz, 2H), 3.77 (s, 3H), 3.66-3.64 (m, 2H), 3.51-3.43 (m, 1H), 3.41-3.38 (m, 2H); LCMS: Rt=0.65 min, m/z=429.1 [M+H].

### Example 245: 5-(4-((1,4-dioxan-2-yl)methoxy)-2-fluorophenyl)-2-oxo-6-(trifluoromethyl)-1,2-dihydropyridine-3-carboxamide (245)

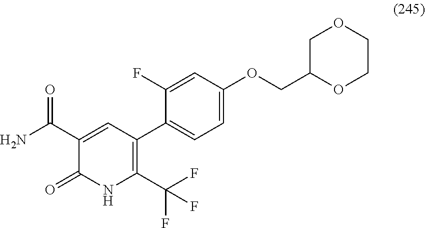 [SMILES: NC(=O)c1cc(-c2ccc(OCC3COCCO3)cc2F)c(C(F)(F)F)[nH]c1=O]

5-(4-((1,4-dioxan-2-yl)methoxy)-2-fluorophenyl)-2-oxo-6-(trifluoromethyl)-1,2-dihydropyridine-3-carboxamide (245) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 4-(4,4,5,5-tetramethyl-1,3,2-dioxaborolan-2-yl)phenol was replaced with 3-fluoro-4-(4,4,5,5-tetramethyl-1,3,2-dioxaborolan-2-yl)phenol, and 1-(4-(hydroxymethyl)piperidin-1-yl)ethan-1-one was replaced with (1,4-dioxan-2-yl)methanol. ¹H NMR (400 MHz, DMSO-d6) δ 9.06 (br. s, 1H), 8.16 (s, 1H), 8.11 (br. s, 1H), 7.28-7.21 (m, 1H), 6.97-6.93 (m, 1H), 6.88-6.86 (m, 1H), 4.04-4.00 (m, 2H), 3.85-3.76 (m, 3H), 3.69-3.65 (m, 2H), 3.64 (m, 1H), 3.43 (m, 1H); LCMS: Rt=0.67 min, m/z=417.1 [M+H].

### Example 246: 5-(4-(morpholin-3-ylmethoxy)phenyl)-2-oxo-6-(trifluoromethyl)-1,2-dihydropyridine-3-carboxamide (246)

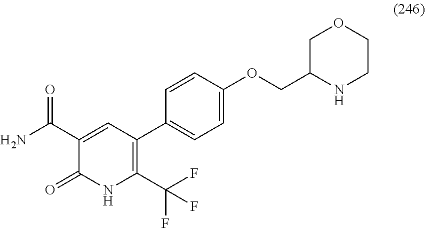 [SMILES: NC(=O)c1cc(-c2ccc(OCC3COCCN3)cc2)c(C(F)(F)F)[nH]c1=O]

5-(4-(morpholin-3-ylmethoxy)phenyl)-2-oxo-6-(trifluoromethyl)-1,2-dihydropyridine-3-carboxamide (246) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with morpholin-3-ylmethanol. ¹H NMR (400 MHz, DMSO-d6) δ 9.09 (br. s, 1H), 8.19 (s, 1H), 8.14 (s, 1H), 7.84 (s, 1H), 7.21 (d, J=8.4 Hz, 2H), 7.02 (d, J=8.4 Hz, 2H), 4.53 (m, 1H), 4.26 (t, J=10.0 Hz, 1H), 3.83 (d, J=9.4 Hz, 2H), 3.72 (d, J=11.2 Hz, 2H), 3.59 (s, 2H), 3.05-3.00 (m, 2H); LCMS: Rt 0.63 min, m/z 398.0 [M+H].

### Example 247: 5-(4-((4-methylmorpholin-3-yl)methoxy)phenyl)-2-oxo-6-(trifluoromethyl)-1,2-dihydropyridine-3-carboxamide (247)

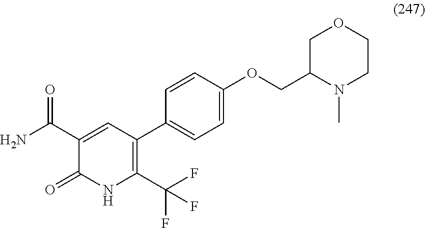 [SMILES: CN1CCOCC1COc1ccc(-c2cc(C(N)=O)c(=O)[nH]c2C(F)(F)F)cc1]

5-(4-((4-methylmorpholin-3-yl)methoxy)phenyl)-2-oxo-6-(trifluoromethyl)-1,2-dihydropyridine-3-carboxamide (247) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-methylmorpholin-3-yl)methanol. ¹H NMR (400 MHz, DMSO-d6) δ 8.73 (s, 1H), 8.21 (s, 1H), 8.15 (s, 1H), 8.03 (s, 1H), 7.25 (d, J=8.6 Hz, 2H), 7.03 (d, J=8.6 Hz, 2H), 4.17 (m, 1H), 3.95 (m, 2H), 3.88 (m, 2H), 3.77-3.69 (m, 1H), 2.77-2.64 (m, 2H), 2.34 (s, 3H), 2.31-2.26 (m, 1H); LCMS: Rt=0.53 min, m/z=412.1 [M+H].

### Example 248: 5-(4-((4-cyclopropylmorpholin-3-yl)methoxy)phenyl)-2-oxo-6-(trifluoromethyl)-1,2-dihydropyridine-3-carboxamide (248)

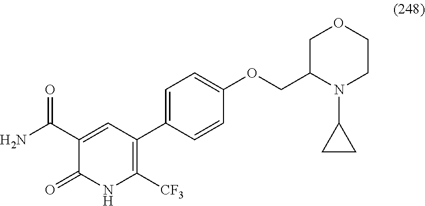 [SMILES: NC(=O)c1cc(-c2ccc(OCC3COCCN3C3CC3)cc2)c(C(F)(F)F)[nH]c1=O]

5-(4-((4-cyclopropylmorpholin-3-yl)methoxy)phenyl)-2-oxo-6-(trifluoromethyl)-1,2-dihydropyridine-3-carboxamide (248) was obtained using a procedure similar to that used to make 5-(4-((4-(cyclopropylmethyl)morpholin-2-yl)methoxy)phenyl)-2-oxo-6-(trifluoromethyl)-1,2-dihydropyridine-3-carboxamide (194), except 2-((4-bromophenoxy)methyl)-4-(cyclopropylmethyl)morpholine was replaced with 3-((4-bromophenoxy)methyl)-4-cyclopropylmorpholine. ¹H NMR (400 MHz, DMSO) δ 8.33 (s, 1H), 7.34 (d, J=8.8 Hz, 2H), 7.12 (d, J=8.8 Hz, 2H), 4.75-4.64 (m, 1H), 4.34 (dd, J=2.0, 11.0 Hz, 1H), 4.18 (dd, J=2.6, 12.6 Hz, 1H), 4.07 (br d, J=12.8 Hz, 1H), 3.96-3.78 (m, 2H), 3.74 (s, 1H), 3.51 (br d, J=12.8 Hz, 1H), 3.03-2.86 (m, 1H), 2.84-2.68 (m, 1H), 1.04-0.79 (m, 4H). LCMS: Rt=0.55 min, m/z=438.2 (M+H).

2-((4-bromophenoxy)methyl)-4-(cyclopropylmethyl)morpholine was obtained using a method similar to that used to make 2-((4-bromophenoxy)methyl)-4-(cyclopropylmethyl)morpholine except cyclopropanone was used in the alkylation step.

### Example 249: 5-(4-((4-ethylmorpholin-3-yl)methoxy)phenyl)-2-oxo-6-(trifluoromethyl)-1,2-dihydropyridine-3-carboxamide (249)

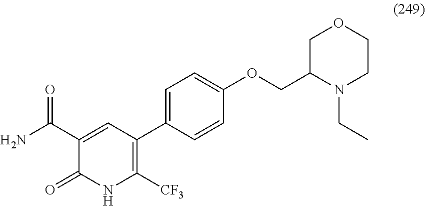 [SMILES: CCN1CCOCC1COc1ccc(-c2cc(C(N)=O)c(=O)[nH]c2C(F)(F)F)cc1]

5-(4-((4-ethylmorpholin-3-ylmethoxy)phenyl)-2-oxo-6-(trifluoromethyl)-1,2-dihydropyridine-3-carboxamide (249) was obtained using a procedure similar to that used to make 5-(4-((4-(cyclopropylmethyl)morpholin-2-yl)methoxy)phenyl)-2-oxo-6-(trifluoromethyl)-1,2-dihydropyridine-3-carboxamide (194), except 2-((4-bromophenoxy)methyl)-4-(cyclopropylmethyl)morpholine was replaced with 3-((4-bromophenoxy)methyl)-4-ethylmorpholine. ¹H NMR (400 MHz, DMSO) δ=8.80 (br, s, 1H), 8.20 (s, 1H), 7.99 (br, s, 1H), 7.26 (d, J=8.6 Hz, 2H), 7.04 (d, J=8.6 Hz, 2H), 4.19 (m, 2H), 3.80 (m, 1H), 3.68 (m, 1H), 3.51 (m, 3H), 2.81 (m, 3H), 2.49 (m, 1H), 1.02 (t, J=7.2 Hz, 3H). LCMS: Rt=0.72 min, m/z=426.4 (M+H).

3-((4-bromophenoxy)methyl)-4-ethylmorpholine was obtained using a method similar to that used to make 2-((4-bromophenoxy)methyl)-4-(cyclopropylmethyl)morpholine except acetaldehyde was used in the alkylation step.

### Example 250: 2-oxo-5-(4-((5-oxomorpholin-3-yl)methoxy)phenyl)-6-(trifluoromethyl)-1,2-dihydropyridine-3-carboxamide (250)

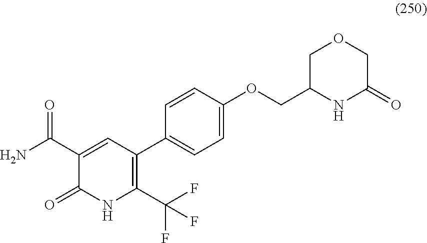 [SMILES: NC(=O)c1cc(-c2ccc(OCC3COCC(=O)N3)cc2)c(C(F)(F)F)[nH]c1=O]

2-oxo-5-(4-((5-oxomorpholin-3-yl)methoxy)phenyl)-6-(trifluoromethyl)-1,2-dihydropyridine-3-carboxamide (250) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 5-(hydroxymethyl)morpholin-3-one. ¹H NMR (400 MHz, DMSO-d6) δ 8.80-8.53 (m, 1H), 8.32-8.17 (m, 2H), 8.07 (s, 1H), 7.28 (d, J=8.4 Hz, 2H), 7.06 (d, J=8.4 Hz, 2H), 4.11-3.98 (m, 4H), 3.88-3.82 (m, 2H), 3.74 (d, J=3.4 Hz, 1H); LCMS: Rt=0.65 min, m/z=412.0 [M+H].

### Example 251: 5-(4-((4-methyl-5-oxomorpholin-3-yl)methoxy)phenyl)-2-oxo-6-(trifluoromethyl)-1,2-dihydropyridine-3-carboxamide (251)

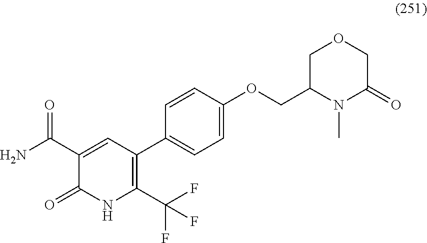 [SMILES: CN1C(=O)COCC1COc1ccc(-c2cc(C(N)=O)c(=O)[nH]c2C(F)(F)F)cc1]

5-(4-((4-methyl-5-oxomorpholin-3-yl)methoxy)phenyl)-2-oxo-6-(trifluoromethyl)-1,2-dihydropyridine-3-carboxamide (251) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 5-(hydroxymethyl)-4-methylmorpholin-3-one. ¹H NMR: (400 MHz, DMSO-d6) δ 8.20 (s, 1H), 8.00 (s, 1H), 7.63-7.39 (m, 1H), 7.20 (d, J=8.4 Hz, 2H), 7.03 (d, J=8.6 Hz, 2H), 4.27-4.14 (m, 2H), 4.08 (d, J=4.6 Hz, 2H), 4.01 (d, J=12.0 Hz, 1H), 3.87 (d, J=12.0 Hz, 1H), 3.74 (d, J=5.0 Hz, 1H), 3.00 (s, 3H); LCMS: Rt=0.68 min, m/z=426.2 [M+H].

### Example 252: 5-(4-((1-(methylsulfonyl)cyclopropyl)methoxy)phenyl)-2-oxo-6-(trifluoromethyl)-1,2-dihydropyridine-3-carboxamide (252)

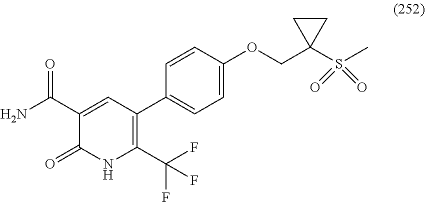 [SMILES: CS(=O)(=O)C1(COc2ccc(-c3cc(C(N)=O)c(=O)[nH]c3C(F)(F)F)cc2)CC1]

5-(4-((1-(methylsulfonyl)cyclopropyl)methoxy)phenyl)-2-oxo-6-(trifluoromethyl)-1,2-dihydropyridine-3-carboxamide (252) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (1-(methylsulfonyl)cyclopropyl)methanol. ¹H NMR (400 MHz, DMSO-d6) δ: 7.95 (s, 1H), 7.40 (d, J=4.4 Hz, 1H), 7.48-7.32 (m, 1H), 7.20 (d, J=8.6 Hz, 2H), 7.01 (d, J=8.8 Hz, 2H), 4.37-4.31 (m, 2H), 3.15 (s, 3H), 1.47-1.39 (m, 2H), 1.27-1.21 (m, 2H); LCMS: Rt=0.72 min, m/z=431.1 [M+H].

### Example 253: 5-(4-((1-(cyclopropylsulfonyl)cyclopropyl)methoxy)phenyl)-2-oxo-6-(trifluoromethyl)-1,2-dihydropyridine-3-carboxamide (253)

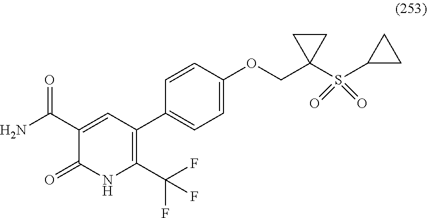 [SMILES: NC(=O)c1cc(-c2ccc(OCC3(S(=O)(=O)C4CC4)CC3)cc2)c(C(F)(F)F)[nH]c1=O]

5-(4-((1-(cyclopropylsulfonyl)cyclopropyl)methoxy)phenyl)-2-oxo-6-(trifluoromethyl)-1,2-dihydropyridine-3-carboxamide (253) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (1-(cyclopropylsulfonyl)cyclopropyl)methanol. ¹H NMR: (400 MHz, DMSO-d6), δ: 13.82-13.47 (m, 1H), 8.75-8.41 (m, 1H), 8.26 (s, 1H), 8.18-8.04 (m, 1H), 7.29 (d, J=8.4 Hz, 2H), 7.08 (d, J=8.6 Hz, 2H), 4.36 (s, 2H), 2.91-2.81 (m, 1H), 1.48-1.38 (m, 2H), 1.29-1.22 (m, 2H), 1.13-0.95 (m, 4H); LCMS: Rt=0.69 min, m/z=457.1 [M+H].

### Example 254: 5-(4-(2-methoxyethoxy)phenyl)-2-oxo-6-(trifluoromethyl)-1,2-dihydropyridine-3-carboxamide (254)

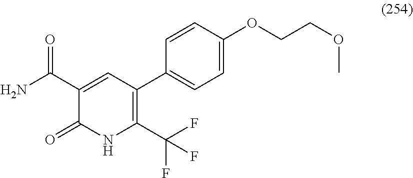 [SMILES: COCCOc1ccc(-c2cc(C(N)=O)c(=O)[nH]c2C(F)(F)F)cc1]

5-(4-(2-methoxyethoxy)phenyl)-2-oxo-6-(trifluoromethyl)-1,2-dihydropyridine-3-carboxamide (254) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 2-methoxyethan-1-ol. LCMS: Rt=0.72 min, m/z=357.3 [M+H].

### Example 255: 5-(4-(2-hydroxyethoxy)phenyl)-2-oxo-6-(trifluoromethyl)-1,2-dihydropyridine-3-carboxamide (255)

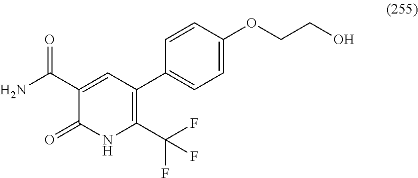 [SMILES: NC(=O)c1cc(-c2ccc(OCCO)cc2)c(C(F)(F)F)[nH]c1=O]

5-(4-(2-hydroxyethoxy)phenyl)-2-oxo-6-(trifluoromethyl)-1,2-dihydropyridine-3-carboxamide (255) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with ethane-1,2-diol. ¹H NMR (500 MHz, DMSO-d6) δ 13.64 (br. s, 1H), 8.47 (br. s, 1H), 8.26 (s, 1H), 8.15 (br. s, 1H), 7.26 (d, J=8.2 Hz, 2H), 7.02 (d, J=8.2 Hz, 2H), 4.89 (t, J=5.5 Hz, 1H), 4.03 (t, J=4.9 Hz, 2H), 3.74 (q, J=5.1 Hz, 2H). LCMS: Rt=1.84 min, m/z=343.1 [M+H].

### Example 256: 5-(4-(2-(methylsulfonyl)ethoxy)phenyl)-2-oxo-6-(trifluoromethyl)-1,2-dihydropyridine-3-carboxamide (256)

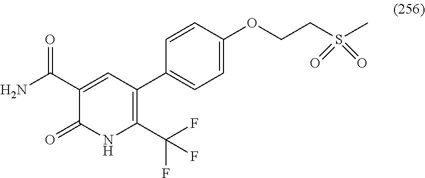 [SMILES: CS(=O)(=O)CCOc1ccc(-c2cc(C(N)=O)c(=O)[nH]c2C(F)(F)F)cc1]

5-(4-(2-(methylsulfonyl)ethoxy)phenyl)-2-oxo-6-(trifluoromethyl)-1,2-dihydropyridine-3-carboxamide (256) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 1-chloro-2-(methylsulfonyl)ethane. ¹H NMR (500 MHz, DMSO-d6) δ 13.64 (br. s, 1H), 8.46 (br. s, 1H), 8.28 (s, 1H), 8.17 (br. s, 1H), 7.40-7.20 (m, 2H), 7.12-7.01 (m, 2H), 4.40 (t, J=5.6 Hz, 2H), 3.65 (t, J=5.6 Hz, 2H), 3.10 (s, 3H). LCMS: Rt=0.62 min, m/z=405.3 [M+H].

### Example 257: 2-oxo-5-(4-(2-(2-oxooxazolidin-3-yl)ethoxy)phenyl)-6-(trifluoromethyl)-1,2-dihydropyridine-3-carboxamide (257)

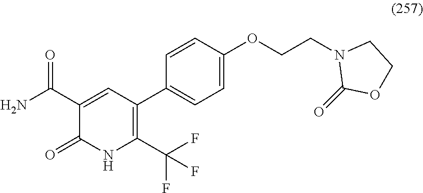 [SMILES: NC(=O)c1cc(-c2ccc(OCCN3CCOC3=O)cc2)c(C(F)(F)F)[nH]c1=O]

2-oxo-5-(4-(2-(2-oxooxazolidin-3-yl)ethoxy)phenyl)-6-(trifluoromethyl)-1,2-dihydropyridine-3-carboxamide (257) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 3-(2-hydroxyethyl)oxazolidin-2-one. ¹H NMR (400 MHz, DMSO-d6) δ 8.53 (br.s, 1H), 8.26 (s, 1H), 8.14 (d, J=3.6 Hz, 1H), 7.29 (d, J=8.8 Hz, 2H), 7.06 (d, J=8.8 Hz, 2H), 4.29-4.19 (m, 2H), 4.18-4.17 (m, 2H), 3.69-3.65 (m, 2H), 3.58-3.55 (m, 2H); LCMS: Rt=0.62 min, m/z=412.1 [M+H].

### Example 258: 5-(4-(2-(1H-imidazol-1-yl)ethoxy)phenyl)-2-oxo-6-(trifluoromethyl)-1,2-dihydropyridine-3-carboxamide (258)

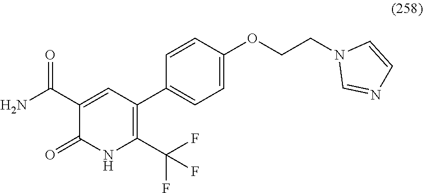 [SMILES: NC(=O)c1cc(-c2ccc(OCCn3ccnc3)cc2)c(C(F)(F)F)[nH]c1=O]

5-(4-(2-(1H-imidazol-1-yl)ethoxy)phenyl)-2-oxo-6-(trifluoromethyl)-1,2-dihydropyridine-3-carboxamide (258) was made using a procedure similar to that used to make 5-(4-((3-nitrobenzyl)oxy)phenyl)-2-oxo-6-(trifluoromethyl-1,2-dihydropyridine-3-carboxamide (54), except 1-(bromomethyl)-3-nitrobenzene was replaced with 1-(2-bromoethyl)-1H-imidazole. LCMS: Rt=0.83 min, m/z=393.3 [M+H].

### Example 259: 5-(4-(2-morpholinoethoxy)phenyl)-2-oxo-6-(trifluoromethyl)-1,2-dihydropyridine-3-carboxamide (259)

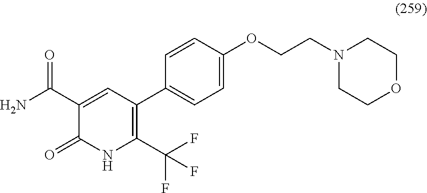 [SMILES: NC(=O)c1cc(-c2ccc(OCCN3CCOCC3)cc2)c(C(F)(F)F)[nH]c1=O]

5-(4-(2-morpholinoethoxy)phenyl)-2-oxo-6-(trifluoromethyl)-1,2-dihydropyridine-3-carboxamide (259) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 2-morpholinoethan-1-ol. LCMS: Rt=0.50 min, m/z=412.4 [M+H].

### Example 260: 5-(4-(2-hydroxy-3-morpholinopropoxy)phenyl)-2-oxo-6-(trifluoromethyl)-1,2-dihydropyridine-3-carboxamide (260)

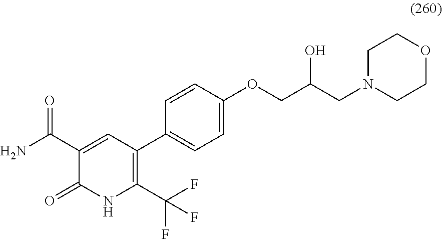 [SMILES: NC(=O)c1cc(-c2ccc(OCC(O)CN3CCOCC3)cc2)c(C(F)(F)F)[nH]c1=O]

−(4-(2-hydroxy-3-morpholinopropoxy)phenyl)-2-oxo-6-(trifluoromethyl)-1,2-dihydropyridine-3-carboxamide (260) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 3-morpholinopropane-1,2-diol. LCMS: Rt=0.60 min, m/z=442.3 [M+H].

### Example 261: 5-(4-((1,3-dimethyl-1H-pyrazol-5-yl)methoxy)phenyl)-2-oxo-6-(trifluoromethyl)-1,2-dihydropyridine-3-carboxamide (261)

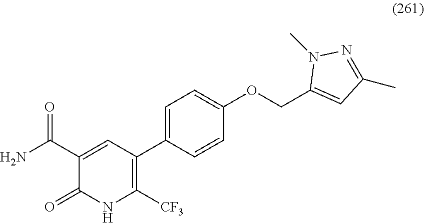 [SMILES: Cc1cc(COc2ccc(-c3cc(C(N)=O)c(=O)[nH]c3C(F)(F)F)cc2)n(C)n1]

5-(4-((1,3-dimethyl-1H-pyrazol-5-yl)methoxy)phenyl)-2-oxo-6-(trifluoromethyl)-1,2-dihydropyridine-3-carboxamide was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-((4-bromophenoxy)methyl)-1,3-dimethyl-1H-pyrazole. ¹H NMR: (400 MHz, DMSO-d6) δ=9.55 (br s, 1H), 8.05 (s, 1H), 7.60 (br s, 1H), 7.23 (d, J=8.4 Hz, 2H), 7.08 (d, J=8.4 Hz, 2H), 6.17 (s, 1H), 5.14 (s, 2H), 3.76 (s, 3H), 2.13 (s, 3H). LCMS: Rt=0.73 min, m/z=407.0 (M+H).

### 5-((4-bromophenoxy)methyl)-1,3-dimethyl-1H-pyrazole was Obtained Using the Following Procedure

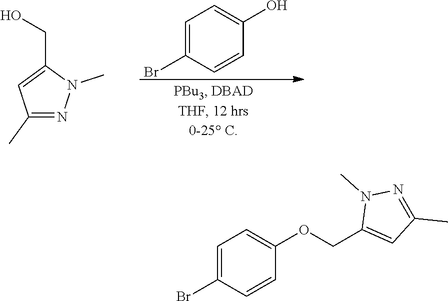

To a solution of (1,3-dimethyl-1H-pyrazol-5-yl)methanol (0.5 g, 3.94 mmol), 4-bromophenol (0.75 g, 4.33 mmol), Bu₃P (1.59 g, 7.88 mmol) in THF (10 mL) was added DBAD (1.59 g, 7.88 mmol) at 0° C. Then the mixture was stirred at 25° C. for 12 hrs. This reaction mixture was concentrated and the residue was purified by column (PE/EA=5/1 to 1/1) to give 5-((4-bromophenoxy)methyl)-1,3-dimethyl-1H-pyrazole. LCMS: Rt=0.83 min, m/z=281.0, 282.9 (M+H).

### Example 262: 5-(4-((3-ethyl-1-methyl-1H-pyrazol-5-yl)methoxy)phenyl)-2-oxo-6-(trifluoromethyl)-1,2-dihydropyridine-3-carboxamide (262)

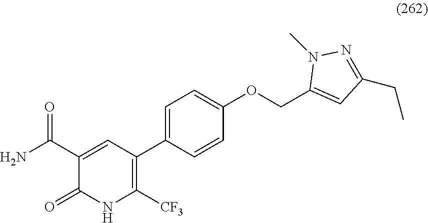 [SMILES: CCc1cc(COc2ccc(-c3cc(C(N)=O)c(=O)[nH]c3C(F)(F)F)cc2)n(C)n1]

5-(4-((3-ethyl-1-methyl-1H-pyrazol-5-yl)methoxy)phenyl)-2-oxo-6-(trifluoromethyl)-1,2-dihydropyridine-3-carboxamide (262) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-((4-bromophenoxy)methyl)-3-ethyl-1-methyl-1H-pyrazole. ¹H NMR (400 MHz, MeOD-d4) δ=8.29 (s, 1H), 7.28 (d, J=8.8 Hz, 2H), 7.11 (d, J=8.8 Hz, 2H), 6.22 (s, 1H), 5.14 (s, 2H), 3.84 (s, 3H), 2.60-2.57 (m, 2H), 1.24 (m, 3H). 19F NMR (377 MHz, MeOD-d4) δ=−62.75 (br s, 3F). LCMS: Rt=0.77 min, m/z=421.0 (M+H).

5-((4-bromophenoxy)methyl)-3-ethyl-1-methyl-1H-pyrazole was obtained using the method for the synthesis of 5-((4-bromophenoxy)methyl)-1,3-dimethyl-1H-pyrazole except (1,3-dimethyl-1H-pyrazol-5-yl)methanol was replaced with (3-ethyl-1-methyl-1H-pyrazol-5-yl)methanol.

### Example 263: 5-(4-((3-cyclopropyl-1-methyl-1H-pyrazol-5-yl)methoxy)phenyl)-2-oxo-6-(trifluoromethyl)-1,2-dihydropyridine-3-carboxamide (263)

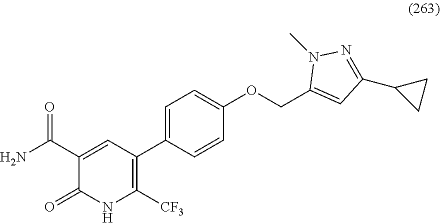 [SMILES: Cn1nc(C2CC2)cc1COc1ccc(-c2cc(C(N)=O)c(=O)[nH]c2C(F)(F)F)cc1]

5-(4-((3-cyclopropyl-1-methyl-1H-pyrazol-5-yl)methoxy)phenyl)-2-oxo-6-(trifluoromethyl)-1,2-dihydropyridine-3-carboxamide (263) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-((4-bromophenoxy)methyl)-3-cyclopropyl-1-methyl-1H-pyrazole. ¹H NMR: (400 MHz, CDCl₃) δ=13.82-13.48 (m, 1H), 8.48 (s, 1H), 8.29 (s, 1H), 8.18 (s, 1H), 7.30 (d, J=8.6 Hz, 2H), 7.12 (d, J=8.8 Hz, 2H), 6.11 (s, 1H), 5.14 (s, 2H), 3.75 (s, 3H), 1.89-1.75 (m, 1H), 0.89-0.76 (m, 2H), 0.65-0.54 (m, 2H). LCMS: Rt=0.72 min, m/z=433.1 (M+H).

5-((4-bromophenoxy)methyl)-3-cyclopropyl-1-methyl-1H-pyrazole was obtained using the method for the synthesis of 5-((4-bromophenoxy)methyl)-1,3-dimethyl-1H-pyrazole except (1,3-dimethyl-1H-pyrazol-5-yl)methanol was replaced with (3-cyclopropyl-1-methyl-1H-pyrazol-5-yl)methanol.

### Example 264: 2-oxo-6-(trifluoromethyl)-5-(4-((1,3,4-trimethyl-1H-pyrazol-5-yl)methoxy)phenyl)-1,2-dihydropyridine-3-carboxamide (264)

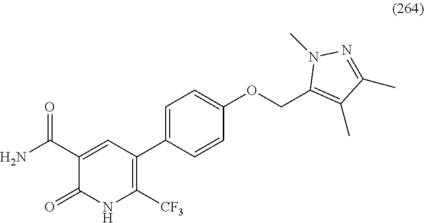 [SMILES: Cc1nn(C)c(COc2ccc(-c3cc(C(N)=O)c(=O)[nH]c3C(F)(F)F)cc2)c1C]

2-oxo-6-(trifluoromethyl)-5-(4-((1,3,4-trimethyl-1H-pyrazol-5-yl)methoxy)phenyl)-1,2-dihydropyridine-3-carboxamide (264) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-((4-bromophenoxy)methyl)-1,3,4-trimethyl-1H-pyrazole. ¹H NMR (400 MHz, MeOD-d4) δ=8.29 (br s, 1H), 7.29 (d, J=8.0 Hz, 5 2H), 7.10 (d, J=8.0 Hz, 2H), 5.09 (s, 2H), 3.80 (s, 3H), 2.15 (s, 3H), 2.02 (s, 3H). 19F NMR (377 MHz, MeOD—−62.72 (br s, 3F). LCMS: Rt=0.75 min, m/z=420.9 (M+H).

5-((4-bromophenoxy)methyl)-1,3,4-trimethyl-1H-pyrazole was obtained using the method for the synthesis of 5-((4-bromophenoxy)methyl)-1,3-dimethyl-1H-pyrazole except (1,3-dimethyl-1H-pyrazol-5-yl)methanol was replaced with (1,3,4-trimethyl-1H-pyrazol-5-yl)methanol.

### Example 265: 5-(4-((1-(cyclopropylmethyl)-1H-pyrazol-5-yl)methoxy)phenyl)-2-oxo-6-(trifluoromethyl)-1,2-dihydropyridine-3-carboxamide (265)

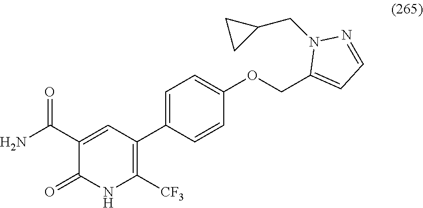 [SMILES: NC(=O)c1cc(-c2ccc(OCc3ccnn3CC3CC3)cc2)c(C(F)(F)F)[nH]c1=O]

5-(4-((1-(cyclopropylmethyl)-1H-pyrazol-5-yl)methoxy)phenyl)-2-oxo-6-(trifluoromethyl)-1,2-dihydropyridine-3-carboxamide (265) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-((4-bromophenoxy)methyl)-1-(cyclopropylmethyl)-1H-pyrazole. ¹H NMR: (400 MHz, MeOD-d4) δ=8.29 (s, 1H), 7.47 (s, 1H), 7.29 (d, J=8.4 Hz, 2H), 7.11 (d, J=8.8 Hz, 2H), 6.43 (s, 1H), 5.21 (s, 2H), 4.09 (d, J=6.8 Hz, 2H), 1.37 (m, 1H), 0.57 (m, 2H), 0.43 (m, 2H). 19F NMR: (377 MHz, MeOD-d4) δ=−62.74 (br s, 3F). LCMS: Rt=0.78 min, m/z=433.0 (M+H).

5-((4-bromophenoxy)methyl)-1-(cyclopropylmethyl)-1H-pyrazole was obtained using the method for the synthesis of 5-((4-bromophenoxy)methyl)-1,3-dimethyl-1H-pyrazole except (1,3-dimethyl-1H-pyrazol-5-yl)methanol was replaced with (1-(cyclopropylmethyl)-1H-pyrazol-5-yl)methanol.

### Example 266: 5-(4-(oxazol-4-ylmethoxy)phenyl)-2-oxo-6-(trifluoromethyl)-1,2-dihydropyridine-3-carboxamide (266)

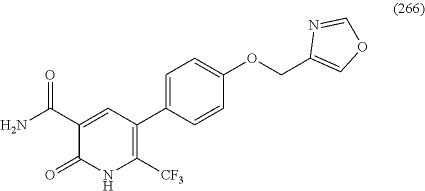 [SMILES: NC(=O)c1cc(-c2ccc(OCc3cocn3)cc2)c(C(F)(F)F)[nH]c1=O]

5-(4-(oxazol-4-ylmethoxy)phenyl)-2-oxo-6-(trifluoromethyl)-1,2-dihydropyridine-3-carboxamide (266) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 4-((4-bromophenoxy)methyl)oxazole. ¹H NMR (400 MHz, DMSO-d6) δ=8.70 (br s, 1H), 8.41 (s, 1H), 8.24 (d, J=4.4 Hz, 2H), 8.04 (br s, 1H), 7.27 (d, J=8.8 Hz, 2H), 7.11 (d, J=8.8 Hz, 2H). 19F NMR (376 MHz, DMSO-d6) δ=−80.40 (br s, 3F). LCMS: Rt=0.79 min, m/z=380.2 (M+H).

### Example 267: 5-(4-(oxazol-5-ylmethoxy)phenyl)-2-oxo-6-(trifluoromethyl)-1,2-dihydropyridine-3-carboxamide (267)

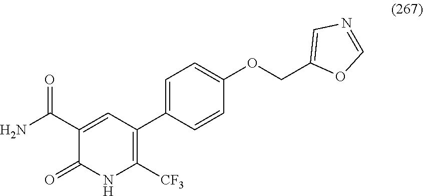 [SMILES: NC(=O)c1cc(-c2ccc(OCc3cnco3)cc2)c(C(F)(F)F)[nH]c1=O]

5-(4-(oxazol-5-ylmethoxy)phenyl)-2-oxo-6-(trifluoromethyl)-1,2-dihydropyridine-3-carboxamide (267) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-(bromomethyl)oxazole. ¹H NMR (400 MHz, DMSO-d6) δ=8.70 (s, 1H), 8.43 (s, 1H), 8.23 (s, 1H), 8.15 (s, 1H), 8.05 (s, 1H), 7.36 (s, 1H), 7.28 (d, J=8.6 Hz, 2H), 7.12 (d, J=8.7 Hz, 2H), 5.24 (s, 2H), 1.18 (d, J=13.7 Hz, 1H). 19F NMR (376 MHz, DMSO-d6) δ=−80.41 (s, 3F). LCMS: Rt=0.79 min, m/z=380.1 (M+H).

### Example 268: 5-(4-((5-cyclopropylisoxazol-3-yl)methoxy)phenyl)-2-oxo-6-(trifluoromethyl)-1,2-dihydropyridine-3-carboxamide (268)

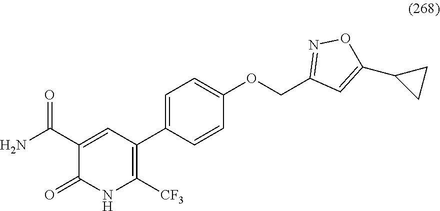 [SMILES: NC(=O)c1cc(-c2ccc(OCc3cc(C4CC4)on3)cc2)c(C(F)(F)F)[nH]c1=O]

5-(4-((5-cyclopropylisoxazol-3-yl)methoxy)phenyl)-2-oxo-6-(trifluoromethyl)-1,2-dihydropyridine-3-carboxamide (268) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 3-(bromomethyl)-5-cyclopropylisoxazole. ¹H NMR (400 MHz, DMSO-d6) δ=9.42 (br s, 1H), 8.18 (s, 1H), 8.06 (m, 1H), 7.66 (m, 1H), 7.24 (br d, J=8.8 Hz, 2H), 7.07 (d, J=8.4 Hz, 2H), 6.33 (s, 1H), 5.15 (s, 2H), 2.18 (m, 1H), 1.09 (m, 2H), 0.91 (m, 2H). LCMS: Rt=0.77 min, m/z=420.1 (M+H).

### Example 269: 5-(4-((5-methylisoxazol-4-yl)methoxy)phenyl)-2-oxo-6-(trifluoromethyl)-1,2-dihydropyridine-3-carboxamide (269)

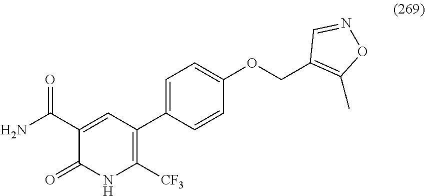 [SMILES: Cc1oncc1COc1ccc(-c2cc(C(N)=O)c(=O)[nH]c2C(F)(F)F)cc1]

5-(4-((5-methylisoxazol-4-yl)methoxy)phenyl)-2-oxo-6-(trifluoromethyl)-1,2-dihydropyridine-3-carboxamide (269) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 4-(bromomethyl)-5-methylisoxazole. ¹H NMR (400 MHz, DMSO-d6) δ=9.35 (br s, 1H), 8.62 (s, 1H), 8.22 (m, 1H), 8.08 (br s, 1H), 7.70 (br s, 1H), 7.23 (d, J=8.4 Hz, 2H), 7.06 (d, J=8.4 Hz, 2H), 4.99 (s, 2H), 5 2.47 (s, 3H). LCMS: Rt=0.72 min, m/z=394.3 (M+H).

### Example 270: 5-(4-((5-methyl-3-(pyridin-4-ylisoxazol-4-yl)methoxy)phenyl)-2-oxo-6-(trifluoromethyl)-1,2-dihydropyridine-3-carboxamide (270)

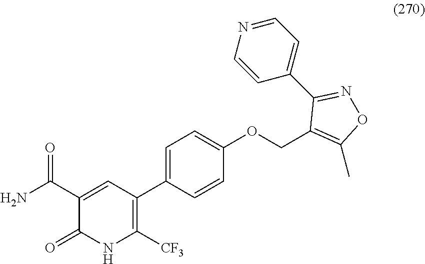 [SMILES: Cc1onc(-c2ccncc2)c1COc1ccc(-c2cc(C(N)=O)c(=O)[nH]c2C(F)(F)F)cc1]

5-(4-((5-methyl-3-(pyridin-4-yl)isoxazol-4-yl)methoxy)phenyl)-2-oxo-6-(trifluoromethyl)-1,2-dihydropyridine-3-carboxamide (270) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 4-(bromomethyl)-5-methyl-3-(pyridin-4-yl)isoxazole. ¹H NMR (400 MHz, DMSO-d6) δ=8.73 (d, J=6.0 Hz, 2H), 8.17 (s, 1H), 8.00 (br s, 1H), 7.72 (d, J=6.0 Hz, 2H), 7.22 (d, J=8.4 Hz, 2H), 7.05 (d, J=8.8 Hz, 2H), 5.09 (s, 2H), 2.56 (s, 3H). 19F NMR (376 MHz, DMSO-d6) δ=−60.29 (br s, 3F). LCMS: Rt=0.70 min, m/z=471.1 (M+H).

### Example 271: 5-(4-((2-methylthiazol-5-yl)methoxy)phenyl)-2-oxo-6-(trifluoromethyl)-1,2-dihydropyridine-3-carboxamide (271)

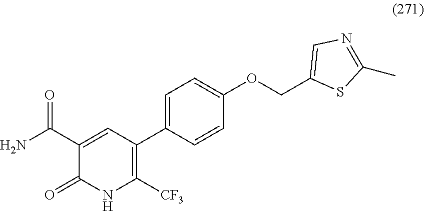 [SMILES: Cc1ncc(COc2ccc(-c3cc(C(N)=O)c(=O)[nH]c3C(F)(F)F)cc2)s1]

5-(4-((2-methylthiazol-5-yl)methoxy)phenyl)-2-oxo-6-(trifluoromethyl)-1,2-dihydropyridine-3-carboxamide (271) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-(bromomethyl)-2-methylthiazole. ¹H NMR (400 MHz, DMSO-d6) δ=10.34 (br s, 1H), 7.89 (s, 1H), 7.73 (s, 1H), 7.21 (br s, 1H), 7.17 (d, J=8.4 Hz, 2H), 7.06-6.99 (m, 2H), 5.32 (s, 2H), 2.70-2.63 (m, 3H). 19F NMR (376 MHz, DMSO-d6) δ=−60.33 (br s, 3F). LCMS: Rt=0.73 min, m/z=410.2 (M+H).

### Example 272: 5-(4-((4-methylthiazol-5-yl)methoxy)phenyl)-2-oxo-6-(trifluoromethyl)-1,2-dihydropyridine-3-carboxamide (272)

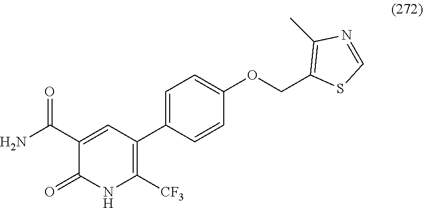 [SMILES: Cc1ncsc1COc1ccc(-c2cc(C(N)=O)c(=O)[nH]c2C(F)(F)F)cc1]

5-(4-((4-methylthiazol-5-yl)methoxy)phenyl)-2-oxo-6-(trifluoromethyl)-1,2-dihydropyridine-3-carboxamide (272) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-(bromomethyl)-4-methylthiazole. ¹H NMR (400 MHz, DMSO-d6) δ=9.00 (s, 1H), 8.10 (br s, 1H), 7.75 (br s, 1H), 7.24 (br d, J=8.4 Hz, 2H), 7.08 (br d, J=8.4 Hz, 2H), 5.33 (s, 2H), 2.43 (s, 3H). 19F NMR (377 MHz, DMSO-d6) δ=−60.35 (br s, 3F). LCMS: Rt=0.80 min, m/z=410.2 (M+H).

### Example 273: 5-(4-((4-methyl-1,2,3-thiadiazol-5-yl)methoxy)phenyl)-2-oxo-6-(trifluoromethyl)-1,2-dihydropyridine-3-carboxamide (273)

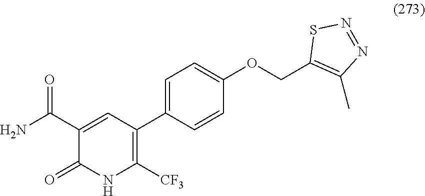 [SMILES: Cc1nnsc1COc1ccc(-c2cc(C(N)=O)c(=O)[nH]c2C(F)(F)F)cc1]

5-(4-((4-methyl-1,2,3-thiadiazol-5-yl)methoxy)phenyl)-2-oxo-6-(trifluoromethyl)-1,2-dihydropyridine-3-carboxamide (273) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-(bromomethyl)-4-methyl-1,2,3-thiadiazole. ¹H NMR (400 MHz, MeOD-d4) δ=8.34-8.23 (m, 1H), 7.31 (br d, J=7.9 Hz, 2H), 7.12 (br d, J=7.8 Hz, 2H), 5.53 (s, 2H), 2.74 (s, 3H); 19F NMR (377 MHz, MeOD-d4) δ=−62.76 (br s, 3F). LCMS: Rt=0.84 min, m/z=411.1 (M+H).

### Example 274: 5-(4-((4-methylpyridin-3-yl)methoxy)phenyl)-2-oxo-6-(trifluoromethyl)-1,2-dihydropyridine-3-carboxamide (274)

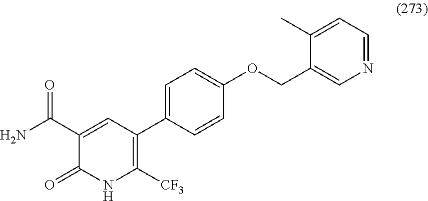 [SMILES: Cc1ccncc1COc1ccc(-c2cc(C(N)=O)c(=O)[nH]c2C(F)(F)F)cc1]

5-(4-((4-methylpyridin-3-yl)methoxy)phenyl)-2-oxo-6-(trifluoromethyl)-1,2-dihydropyridine-3-carboxamide (274) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 3-(bromomethyl)-4-methylpyridine. ¹H NMR (400 MHz, DMSO-d6) δ=9.06 (br s, 1H), 8.59 (s, 1H), 8.44 (d, J=5.2 Hz, 1H), 8.17 (s, 1H), 7.86 (br s, 1H), 7.29 (m, 2H), 7.26 (s, 1H), 7.14 (d, J=8.8 Hz, 2H), 5.20 (s, 2H), 2.50 (s, 3H); LCMS: Rt=0.58 min, m/z=404.1 (M+H).

### Example 275: 5-(4-((4-cyclopropylpyridin-3-yl)methoxy)phenyl)-2-oxo-6-(trifluoromethyl)-1,2-dihydropyridine-3-carboxamide (275)

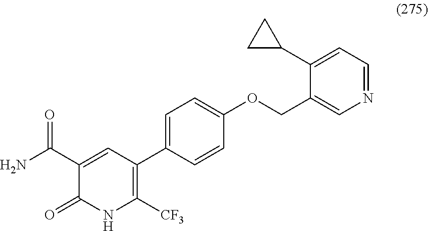 [SMILES: NC(=O)c1cc(-c2ccc(OCc3cnccc3C3CC3)cc2)c(C(F)(F)F)[nH]c1=O]

5-(4-((4-cyclopropylpyridin-3-yl)methoxy)phenyl)-2-oxo-6-(trifluoromethyl)-1,2-dihydropyridine-3-carboxamide (275) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-cyclopropylpyridin-3-yl)methanol. ¹H NMR (400 MHz, DMSO-d6) δ=10.81 (br s, 1H), 8.56 (s, 1H), 8.42 (d, J=5.2 Hz, 1H), 7.80 (s, 1H), 7.17 (m, 2H), 7.07 (m, 2H), 7.01 (m, 1H), 6.94 (d, J=5.2 Hz, 1H), 5.27 (s, 2H), 2.32 (m, 1H), 1.09 (m, 2H), 0.83 (m, 2H). LCMS: Rt=0.61 min, m/z=430.1 (M+H).

### (4-cyclopropylpyridin-3-yl)methanol was Obtained Using the Following Procedure

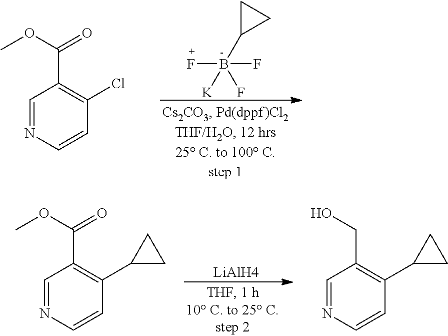

Step 1: To a solution of methyl 4-chloronicotinate, (900 mg, 5.26 mmol) in THF (10 mL) and H₂O (2 mL) was added potassium cyclopropyltrifluoroborate (818 mg, 5.53 mmol), Cs₂CO₃ (4.8 g, 14.73 mmol) and Pd(dppf)Cl₂ (385 mg, 0.53 mmol) at 25° C. under N₂. The mixture was stirred at 100° C. for 12 hrs. The mixture was filtered, concentrated and purified by column chromatography (PE/EA=3/1) to give methyl 4-cyclopropylnicotinate. ¹H NMR (400 MHz, CDCl₃) δ=9.01 (s, 1H), 8.55 (d, J=5.4 Hz, 1H), 6.83 (d, J=5.4 Hz, 1H), 3.97 (s, 3H), 2.96 (m, 1H), 1.23 (m, 2H), 0.87 (m, 2H)

Step 2: To a solution of methyl 4-cyclopropylnicotinate, (500 mg, 2.82 mmol) in THF (10 mL) was added LiAlH₄ (107 mg, 2.82 mmol) at 10° C. The mixture was stirred 25° C. for 1 hr. Water (0.11 mL) was added to the mixture, followed by the addition of 15% aq. NaOH (0.11 mL), then followed by the addition of water (0.33 mL) and sodium sulfate (0.5 g). The mixture was stirred for 30 min. The mixture was filtered and the filtrate was washed with EA (20 mL×2). The combined organic layers were dried over anhydrous Na₂SO₄, filtered and concentrated to give (4-cyclopropylpyridin-3-yl)methanol. ¹H NMR (400 MHz, CDCl₃) δ=8.47 (s, 1H), 8.42 (m, 1H), 6.79 (d, J=5.0 Hz, 1H), 4.89 (s, 2H), 2.16 (m, 1H), 1.26 (m, 2H), 0.80 (m, 3H).

### Example 276: 5-(4-((4-methoxypyridin-3-yl)methoxy)phenyl)-2-oxo-6-(trifluoromethyl)-1,2-dihydropyridine-3-carboxamide (276)

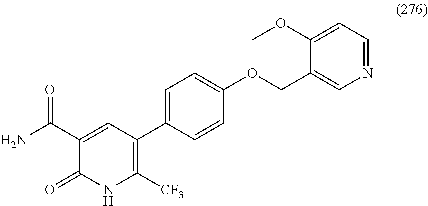 [SMILES: COc1ccncc1COc1ccc(-c2cc(C(N)=O)c(=O)[nH]c2C(F)(F)F)cc1]

5-(4-((4-methoxypyridin-3-yl)methoxy)phenyl)-2-oxo-6-(trifluoromethyl)-1,2-dihydropyridine-3-carboxamide (276) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-methoxypyridin-3-yl)methanol. ¹H NMR (400 MHz, DMSO-d6) δ=9.09 (br s, 1H), 8.49 (m, 2H), 8.17 (s, 1H), 8.14 (s, 1H), 7.80 (br s, 1H), 7.25 (d, J=8.6 Hz, 2H), 7.14 (d, J=5.8 Hz, 1H), 7.09 (d, J=8.6 Hz, 2H), 5.11 (s, 2H), 3.91 (s, 3H). LCMS: Rt=0.58 min, m/z=420.1 (M+H).

### Example 277: 5-(4-((4-ethoxypyridin-3-yl)methoxy)phenyl)-2-oxo-6-(trifluoromethyl)-1,2-dihydropyridine-3-carboxamide (277)

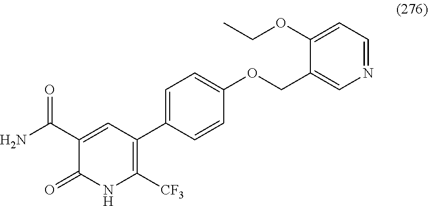 [SMILES: CCOc1ccncc1COc1ccc(-c2cc(C(N)=O)c(=O)[nH]c2C(F)(F)F)cc1]

5-(4-((4-ethoxypyridin-3-yl)methoxy)phenyl)-2-oxo-6-(trifluoromethyl)-1,2-dihydropyridine-3-carboxamide (277) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-ethoxypyridin-3-yl)methanol. ¹H NMR (400 MHz, DMSO-d6) δ=10.84 (s, 1H), 8.48 (s, 1H), 8.43 (d, J=4.0 Hz, 1H), 7.79 (s, 1H), 7.13 (d, J=8.0 Hz, 2H), 7.09 (d, J=4.0 Hz, 1H), 7.00 (d, J=8.0 Hz, 2H), 6.95 (s, 1H), 5.07 (s, 2H), 4.19 (m, 2H), 1.34 (t, J=4.0 Hz, 3H). LCMS: Rt=0.74 min, m/z=434.2 (M+H).

### Example 278: 5-(4-((5-methylpyridin-3-yl)methoxy)phenyl)-2-oxo-6-(trifluoromethyl)-1,2-dihydropyridine-3-carboxamide (278)

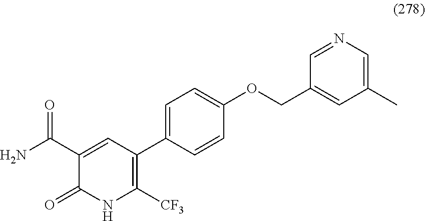 [SMILES: Cc1cncc(COc2ccc(-c3cc(C(N)=O)c(=O)[nH]c3C(F)(F)F)cc2)c1]

5-(4-((5-methylpyridin-3-yl)methoxy)phenyl)-2-oxo-6-(trifluoromethyl)-1,2-dihydropyridine-3-carboxamide (278) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-methylpyridin-3-yl)methanol. ¹H NMR (400 MHz, DMSO-d6) δ=10.34 (br s, 1H), 8.50 (d, J=0.9 Hz, 1H), 8.40 (s, 1H), 7.89 (br d, J=2.8 Hz, 1H), 7.72 (s, 1H), 7.23 (br s, 1H), 7.18 (br d, J=8.4 Hz, 2H), 7.04 (d, J=8.8 Hz, 2H), 2.50 (s, 3H). LCMS: Rt=0.620 min, m/z=404.0 (M+H).

### Example 279: 5-(4-((5-cyclopropylpyridin-3-yl)methoxy)phenyl)-2-oxo-6-(trifluoromethyl)-1,2-dihydropyridine-3-carboxamide (279)

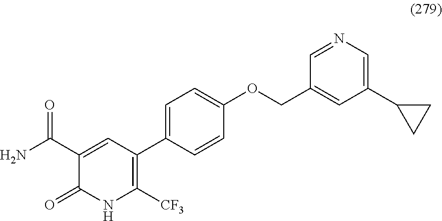 [SMILES: NC(=O)c1cc(-c2ccc(OCc3cncc(C4CC4)c3)cc2)c(C(F)(F)F)[nH]c1=O]

5-(4-((5-cyclopropylpyridin-3-yl)methoxy)phenyl)-2-oxo-6-(trifluoromethyl)-1,2-dihydropyridine-3-carboxamide (279) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-cyclopropylpyridin-3-yl)methanol. ¹H NMR (400 MHz, DMSO-d6) δ=10.04 (m, 1H), 8.45 (d, J=1.6 Hz, 1H), 8.37 (m, 1H), 8.18 (br s, 1H), 10 7.93 (br s, 1H), 7.52 (s, 1H), 7.37 (m, 1H), 7.19 (br d, J=8.0 Hz, 2H), 7.04 (br d, J=8.4 Hz, 2H), 5.11 (s, 2H), 2.32 (m, 1H), 1.02 (m, 2H), 0.99 (m, 2H). LCMS: Rt=0.62 min, m/z=430.1 (M+H).

### (5-cyclopropylpyridin-3-yl)methanol was Obtained Using the Following Procedure

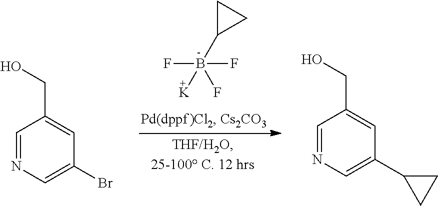

Pd(dppf)Cl₂ (434 mg, 0.532 mmol) was added to a solution of (5-bromopyridin-3-yl)methanol (1.0 g, 5.32 mmol, potassium cyclopropyltrifluoroborate (1.57 g, 10.64 mmol) and Cs₂CO₃ (4.85 g, 14.896 mmol) in 10:1 THF/H₂O (22 mL) at 25° C. under N₂ and the mixture was stirred at 100° C. for 12 hrs. The mixture was purified by column (PE-PE/EA=1:1) directly to give (5-cyclopropylpyridin-3-yl)methanol. ¹H NMR (400 MHz, CDCl₃) δ 8.31 (m, 2H), 7.35 (br s, 1H), 4.72 (s, 2H), 1.89 (m, 1H), 1.03 (m, 3H), 0.73 (m, 3H).

### Example 280: 5-(4-((4-isopropoxypyridin-3-yl)methoxy)phenyl)-2-oxo-6-(trifluoromethyl)-1,2-dihydropyridine-3-carboxamide (280)

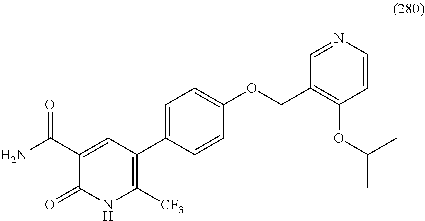 [SMILES: CC(C)Oc1ccncc1COc1ccc(-c2cc(C(N)=O)c(=O)[nH]c2C(F)(F)F)cc1]

5-(4-((4-isopropoxypyridin-3-yl)methoxy)phenyl)-2-oxo-6-(trifluoromethyl)-1,2-dihydropyridine-3-carboxamide (280) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-isopropoxypyridin-3-yl)methanol. ¹H NMR (400 MHz, DMSO-d6) δ=8.59-8.46 (m, 2H), 8.42 (s, 1H), 8.27 (s, 1H), 8.14 (s, 1H), 7.28 (d, J=8.0 Hz, 2H), 7.14 (s, 1H), 7.10 (d, J=8.0 Hz, 2H), 5.10 (s, 2H), 4.82 (m, 1H), 1.29 (d, J=6.0 Hz, 6H). LCMS: Rt=0.67 min, m/z=448.1 (M+H).

### Example 281: 5-(4-((4-methylpyrimidin-5-yl)methoxy)phenyl)-2-oxo-6-(trifluoromethyl)-1,2-dihydropyridine-3-carboxamide (281)

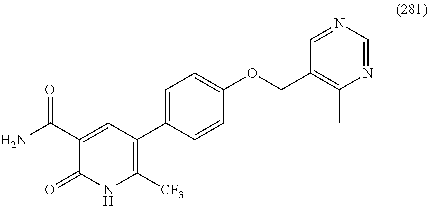 [SMILES: Cc1ncncc1COc1ccc(-c2cc(C(N)=O)c(=O)[nH]c2C(F)(F)F)cc1]

5-(4-((4-methylpyrimidin-5-yl)methoxy)phenyl)-2-oxo-6-(trifluoromethyl)-1,2-dihydropyridine-3-carboxamide (281) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-methylpyrimidin-5-yl)methanol. ¹H NMR (500 MHz, DMSO-d6) δ=13.65 (s, 1H), 9.05 (s, 1H), 8.80 (s, 1H), 8.54 (br s, 1H), 8.29 (s, 1H), 8.16 (br s, 1H), 7.33 (d, J=8.7 Hz, 2H), 7.19 (d, J=8.8 Hz, 2H), 5.26 (s, 2H), 2.57 (s, 3H). LCMS: Rt=2.53 min, m/z=405.0 (M+H).

### Example 282: 5-(4-((4-cyclopropylpyrimidin-5-yl)methoxy)phenyl)-2-oxo-6-(trifluoromethyl)-1,2-dihydropyridine-3-carboxamide (282)

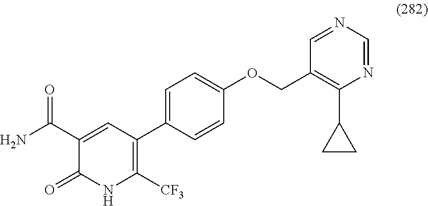 [SMILES: NC(=O)c1cc(-c2ccc(OCc3cncnc3C3CC3)cc2)c(C(F)(F)F)[nH]c1=O]

5-(4-((4-cyclopropylpyrimidin-5-yl)methoxy)phenyl)-2-oxo-6-(trifluoromethyl)-1,2-dihydropyridine-3-carboxamide (282) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4-cyclopropylpyrimidin-5-yl)methanol. ¹H NMR: (500 MHz, DMSO-d6) δ=13.66 (s, 1H), 8.98 (s, 1H), 8.75 (s, 1H), 8.47 (s, 1H), 8.30 (s, 1H), 8.20 (s, 1H), 7.34 (d, J=8.6 Hz, 2H), 7.21 (d, J=8.7 Hz, 2H), 5.36 (s, 2H), 2.36-2.30 (m, 1H), 1.17-1.10 (m, 4H). LCMS: Rt=3.02 min, m/z=430.9 (M+H).

### Example 283: 5-(4-((6-methylpyrazin-2-yl)methoxy)phenyl)-2-oxo-6-(trifluoromethyl)-1,2-dihydropyridine-3-carboxamide (283)

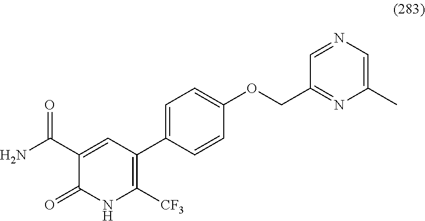 [SMILES: Cc1cncc(COc2ccc(-c3cc(C(N)=O)c(=O)[nH]c3C(F)(F)F)cc2)n1]

5-(4-((6-methylpyrazin-2-yl)methoxy)phenyl)-2-oxo-6-(trifluoromethyl)-1,2-dihydropyridine-3-carboxamide (283) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (6-methylpyrazin-2-yl)methanol. ¹H NMR (400 MHz, DMSO-d6) δ=8.85 (m, 1H), 8.62 (s, 1H), 8.54 (s, 1H), 8.23 (s, 1H), 8.14 (s, 1H), 8.03 (br s, 1H), 7.29 (d, J=8.8 Hz, 2H), 7.14 (d, J=8.8 Hz, 2H), 5.26 (s, 2H), 2.53 (s, 3H). 19F NMR (376 MHz, DMSO-d6) δ=−60.40 (br s, 3F). LCMS: Rt=0.81 min, m/z=443.2 (M+K).

### Example 284: 5-(4-((6-cyclopropylpyrazin-2-yl)methoxy)phenyl)-2-oxo-6-(trifluoromethyl)-1,2-dihydropyridine-3-carboxamide (284)

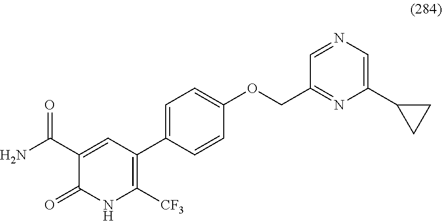 [SMILES: NC(=O)c1cc(-c2ccc(OCc3cncc(C4CC4)n3)cc2)c(C(F)(F)F)[nH]c1=O]

5-(4-((6-cyclopropylpyrazin-2-yl)methoxy)phenyl)-2-oxo-6-(trifluoromethyl)-1,2-dihydropyridine-3-carboxamide (284) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (6-cyclopropylpyrazin-2-yl)methanol. ¹H NMR (400 MHz, MeOD-d4) δ=8.50 (s, 1H), 8.46 (s, 1H), 8.41 (s, 1H), 8.24 (br s, 1H), 7.27 (d, J=8.4 Hz, 2H), 7.10 (d, J=8.4 Hz, 2H), 5.22 (s, 2H), 2.23 (m, 1H), 1.10-1.08 (m, 4H); 19F NMR: (376 MHz, MeOD-d4) δ=−62.85 (br s, 3F). LCMS: Rt=0.80 min, m/z 431.0 (M+H).

### (6-cyclopropylpyrazin-2-yl)methanol was Obtained Using the Following Procedure

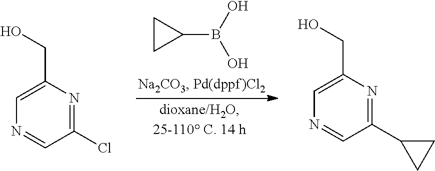 [SMILES: OCc1cncc(Cl)n1 ; OB(O)C1CC1 ; OCc1cncc(C2CC2)n1]

To a solution of (6-chloropyrazin-2-yl)methanol (500 mg, 3.5 mmol) in dioxane/H₂O (25 mL) was added cyclopropylboronic acid (360 mg, 4.2 mmol, Na₂CO₃ (1.11 g, 10.5 mmol), Pd(dppf)Cl₂ (510 mg, 0.7 mmol) at 25° C. under N₂, then stirred at 110° C. for 14 h. The reaction mixture was concentrated, then purified by Prep-HPLC (condition: FA) to give (6-cyclopropylpyrazin-2-yl)methanol. LCMS: Rt=0.30 min, m/z=151.1 (M+H); Rt=0.312 min, m/z=151.1 (M+H). ¹H NMR (400 MHz, MeOD-d4) δ=8.40 (d, J=17.2 Hz, 2H), 4.66 (s, 2H), 2.13 (m, 1H), 1.04-1.02 (m, 4H).

### Example 285: 5-(4-(imidazo[1,2-a]pyridin-2-ylmethoxy)phenyl)-2-oxo-6-(trifluoromethyl)-1,2-dihydropyridine-3-carboxamide (285)

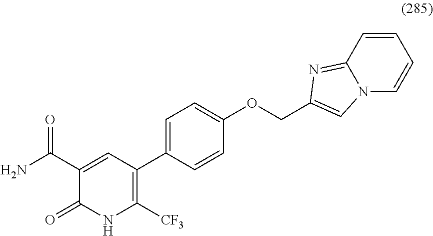 [SMILES: NC(=O)c1cc(-c2ccc(OCc3cn4ccccc4n3)cc2)c(C(F)(F)F)[nH]c1=O]

5-(4-(imidazo[1,2-a]pyridin-2-ylmethoxy)phenyl)-2-oxo-6-(trifluoromethyl)-1,2-dihydropyridine-3-carboxamide (285) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with imidazo[1,2-a]pyridin-2-ylmethanol. ¹H NMR (400 MHz, DMSO-d6) δ=8.55 (d, J=6.8 Hz, 1H), 8.15-7.98 (m, 2H), 7.55 (d, J=9.0 Hz, 2H), 7.31-7.19 (m, 3H), 7.10 (d, J=8.7 Hz, 2H), 6.90 (t, J=6.8 Hz, 1H), 5.24 (s, 2H). LCMS: Rt=0.58 min, m/z=429.1 (M+H).

### Example 286: 5-(4-(imidazo[1,2-a]pyridin-3-ylmethoxy)phenyl)-2-oxo-6-(trifluoromethyl)-1,2-dihydropyridine-3-carboxamide (286)

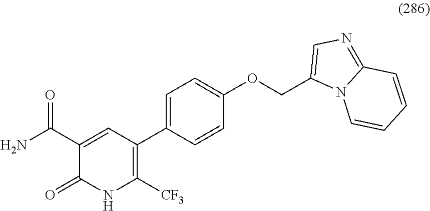 [SMILES: NC(=O)c1cc(-c2ccc(OCc3cnc4ccccn34)cc2)c(C(F)(F)F)[nH]c1=O]

5-(4-(imidazo[1,2-a]pyridin-3-ylmethoxy)phenyl)-2-oxo-6-(trifluoromethyl)-1,2-dihydropyridine-3-carboxamide (286) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with imidazo[1,2-a]pyridin-3-ylmethanol. ¹H NMR (400 MHz, DMSO-d6) δ=8.98 (br s, 1H), 8.49 (d, J=6.8 Hz, 1H), 8.16 (s, 2H), 7.89 (br s, 1H), 7.78 (s, 1H), 7.64 (br d, J=9.0 Hz, 1H), 7.34 (m, 1H), 7.27 (d, J=8.4 Hz, 2H), 7.16 (br d, J=8.4 Hz, 2H), 7.02 (m, 1H), 5.54 (s, 2H). LCMS: Rt=0.73 min, m/z=429.2 (M+Na).

### Example 287: 2-oxo-5-(4-((4,5,6,7-tetrahydrobenzo[d]isoxazol-3-ylmethoxy)phenyl)-6-(trifluoromethyl)-1,2-dihydropyridine-3-carboxamide (287)

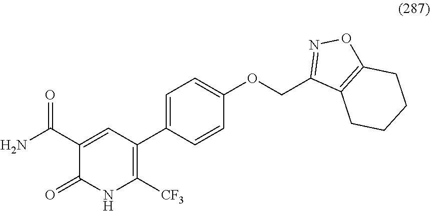 [SMILES: NC(=O)c1cc(-c2ccc(OCc3noc4c3CCCC4)cc2)c(C(F)(F)F)[nH]c1=O]

2-oxo-5-(4-((4,5,6,7-tetrahydrobenzo[d]isoxazol-3-yl)methoxy)phenyl)-6-(trifluoromethyl)-1,2-dihydropyridine-3-carboxamide (287) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4,5,6,7-tetrahydrobenzo[d]isoxazol-3-yl)methanol. ¹H NMR (400 MHz, DMSO-d6) δ=8.56 (s, 1H), 8.26 (s, 1H), 8.20-8.04 (m, 1H), 7.29 (d, J=8.0 Hz, 2H), 7.11 (d, J=8.0 Hz, 2H), 5.21 (s, 2H), 2.69-2.66 (m, 2H), 2.47-2.40 (m, 2H), 1.86-1.74 (m, 2H), 1.74-1.65 (m, 2H). LCMS: Rt=0.83 min, m/z=434.1 (M+H).

### Example 288: 5-(4-((2,3-dihydrobenzofuran-2-yl)methoxy)phenyl)-2-oxo-6-(trifluoromethyl)-1,2-dihydropyridine-3-carboxamide (288)

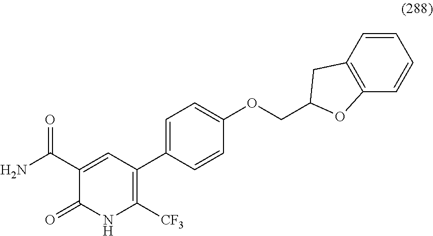 [SMILES: NC(=O)c1cc(-c2ccc(OCC3Cc4ccccc4O3)cc2)c(C(F)(F)F)[nH]c1=O]

5-(4-((2,3-dihydrobenzofuran-2-yl)methoxy)phenyl)-2-oxo-6-(trifluoromethyl)-1,2-dihydropyridine-3-carboxamide (288) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (2,3-dihydrobenzofuran-2-yl)methanol. ¹H NMR (400 MHz, DMSO-d6) δ 13.60 (m, 1H), 8.63 (m, 1H), 8.24 (s, 1H), 8.09 (br s, 1H), 7.27 (m, 3H), 7.07 (m, 3H), 6.83 (m, 2H), 5.16 (m, 1H), 4.25 (m, 2H), 3.40 (m, 1H), 5 3.11 (m, 1H). LCMS: Rt=0.80 min, m/z=431.3 (M+Na).

### Example 289: 5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carboxamide (SFC Peak 4) (289)

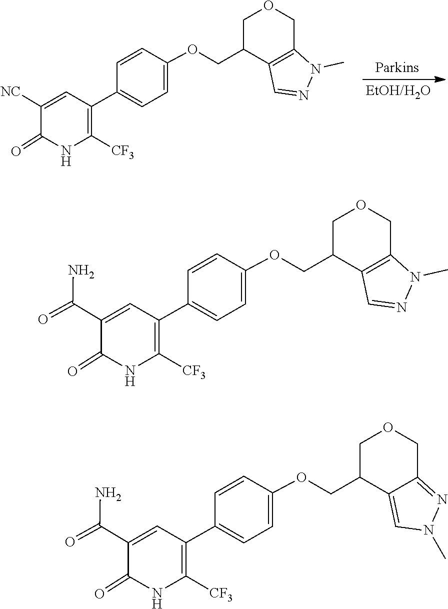 [SMILES: Cn1ncc2c1COCC2COc1ccc(-c2cc(C#N)c(=O)[nH]c2C(F)(F)F)cc1 ; Cn1ncc2c1COCC2COc1ccc(-c2cc(C(N)=O)c(=O)[nH]c2C(F)(F)F)cc1 ; Cn1cc2c(n1)COCC2COc1ccc(-c2cc(C(N)=O)c(=O)[nH]c2C(F)(F)F)cc1]

Parkins catalyst (5 mg, 0.05 mmol) was added to a solution of 5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carbonitrile (95 mg, 0.22 mmol) in EtOH/H₂O 9:1 (2 mL) at 25° C. and the mixture stirred at 70° C. for 4 hrs under N₂. The mixture was filtered and concentrated and the residue purified by reversed-phase column (NH₃—H₂O) to give crude product. The crude product was purified by SFC to afford four individual isomers.

5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carboxamide (SFC peak 4) (289): ¹H NMR: (400 MHz, DMSO-d6), δ=9.37 (br s, 1H), 8.10 (s, 1H), 7.74 (br s, 1H), 7.45 (s, 1H), 7.24 (d, J=8.4, 2H), 7.05 (d, J=8.8, 2H), 4.80 (m, 2H), 4.09 (m, 2H), 4.01 (m, 2H), 3.84 (m, 3H), 3.68 (m, 1H). LCMS: Rt=0.73 min, m/z=449.1 (M+H). Chiral HPLC: Rt=1.20 min, ee value=100%. Chiralpak AD-3 100×4.6 mm I.D

### 5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carbonitrile was Obtained Using the Following Procedure

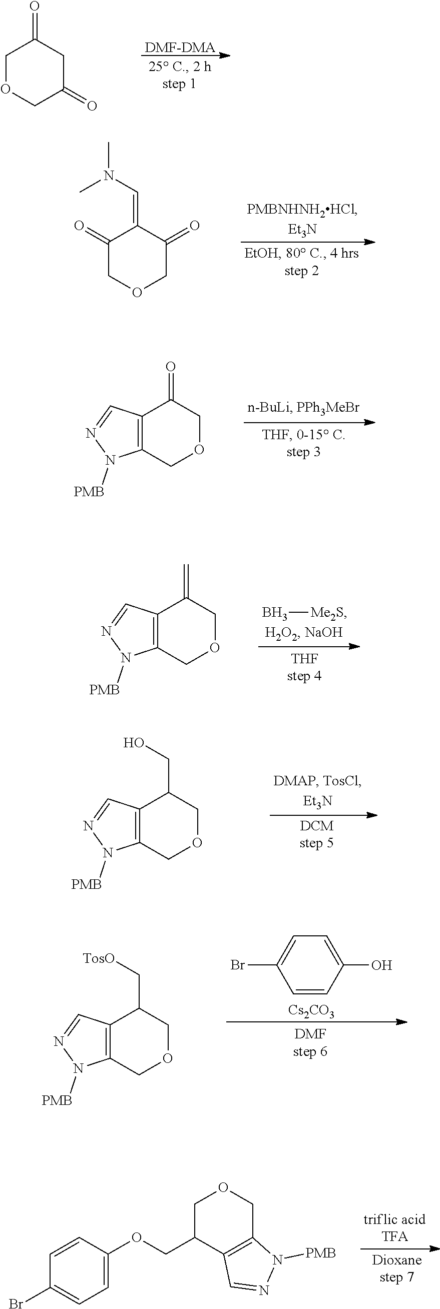

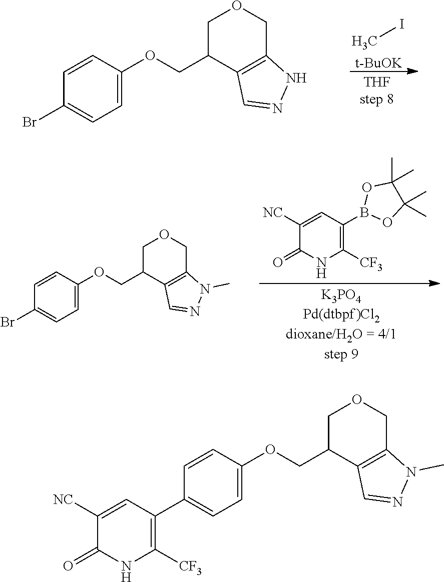

Step 1: 2H-pyran-3,5(4H,6H)-dione (14 g, 122.7 mmol) was added to DMF-DMA (49 mL) at 25° C. The mixture was stirred at 25° C. for 2 hrs. The reaction mixture was filtered to give a filter cake and the filter cake was washed with PE:EA=10:1 (100 mL) to give crude product. Then EtOAC (150 mL) was added to the crude product and the mixture was stirred for 10 min. The mixture was filtered, and the filtrate was concentrated in vacuo to give 4-((dimethylamino)methylene)-2H-pyran-3,5(4H,6H)-dione. TLC: (PE:EA=3:1, Rf=0.3). ¹H NMR (400 MHz, DMSO-d6) δ=8.07 (s, 1H), 4.01 (s, 4H), 3.44 (s, 3H), 3.14 (s, 3H).

Step 2: To a solution of 4-((dimethylamino)methylene)-2H-pyran-3,5(4H,6H)-dione (10.5 g, 62.06 mmol), PMBNHNH₂—HCl (14 g, 74.47 mmol) in EtOH (250 mL) was added Et₃N (12.6 g, 124.12 mmol) and the mixture stirred at 80° C. for 4 hrs. The mixture was concentrated to give a residue which was subjected to silica gel chromotography (PE/EA=1/1) to give crude product. The crude product was washed with a solution of PE/EA (v/v=2:1, 200 mL) to give 1-(4-methoxybenzyl)-1,7-dihydropyrano[3,4-c]pyrazol-4(5H)-one. TLC:PE/EA=0/1, Rf=0.7. ¹H NMR (400 MHz, DMSO-d6) δ=7.93 (s, 1H), 7.23-7.21 (m, 2H), 6.92-6.90 (m, 2H), 5.28 (s, 2H), 54.95-4.94 (m, 2H), 4.07 (s, 2H), 3.73 (s, 3H).

Step 3: To a solution under N₂ of bromo(methyl)triphenylphosphorane (17.9 g, 50 mmol) in dry THF (80 mL) at 0° C. was added n-BuLi (2.5M, 20 ml, 50 mmol). The resulting yellow mixture was stirred for 0.5 hr at 0° C., then a solution of 1-(4-methoxybenzyl)-1,7-dihydropyrano[3,4-c]pyrazol-4(5H)-one (2.6 g, 10 mmol) in dry THF (20 mL) was added dropwise. The reaction was stirred at 15° C. for 3.5 hrs. The reaction was then quenched by the addition of H₂O (500 mL) and extracted with EtOAc (3×200 mL). The combined organic layers were dried over Na₂SO₄, filtered, and concentrated in vacuo to give a residue which was purified by column chromatography (PE/EA=5/1) to give 1-(4-methoxybenzyl)-4-methylene-1,4,5,7-tetrahydropyrano[3,4-c]pyrazole. LCMS: Rt=0.76 min m/z=257.1 (M+H). ¹H NMR (400 MHz, CDCl₃) δ=7.68 (s, 1H), 7.27-7.08 (m, 2H), 6.88-6.85 (m, 2H), 5.15-5.14 (m, 3H), 4.82 (s, 1H), 4.56 (s, 2H), 4.21 (s, 2H), 3.79 (s, 3H).

Step 4: To a solution of 1-(4-methoxybenzyl)-4-methylene-1,4,5,7-tetrahydropyrano[3,4-c]pyrazole (1.3 g, 5.07 mmol) in THF (30 mL) was added BH₃-Me₂S (1.02 mL, 10.14 mmol, 10M) dropwise at 0° C. under N₂. The mixture was stirred at 0° C. for 0.5 hour, then warmed to 20° C. and stirred at 20° C. for 15 hrs. Aq. NaOH (10.2 mL, 30.42 mmol, 3M) and H₂O₂ (10.2 mL, 30.42 mmol, 30% concentration) were added at 0° C. and the mixture stirred at 0° C. for 0.5 hour, followed by stirring at 20° C. for 42 hrs. K₂CO₃ (6 g) was added and the mixture was stirred for 2 hrs. Saturated Na₂SO₃ (aq. 200 mL) was added to the reaction solution sat 0° C. and the mixture was extracted with EtOAc (3×30 mL). The organic layers were combined and dried over Na₂SO₄, filtered and concentrated to give a residue which was purified by silica gel chromatography (PE-EA) to give (1-(4-methoxybenzyl)-1,4,5,7-tetrahydropyrano[3,4-c]pyrazol-4-yl)methanol. LCMS: Rt=0.63 min, m/z=275.1 (M+H). ¹H NMR (400 MHz, CDCl₃) δ=7.12 (s, 1H), 7.10-7.08 (m, 2H), 6.86-6.84 (m, 2H), 5.20-5.02 (m, 2H), 4.66-4.31 (m, 2H), 3.90-3.87 (m, 1H), 3.78-3.75 (m, 2H), 3.73-3.71 (m, 2H), 2.98 (s, 5H), 2.95-2.84 (m, 1H).

Step 5: To a solution of (1-(4-methoxybenzyl)-1,4,5,7-tetrahydropyrano[3,4-c]pyrazol-4-yl)methanol (580 mg, 2.11 mmol, DMAP (129 mg, 1.06 mmol) and Et₃N (427 mg, 4.22 mmol) in DCM (10 mL) was added TosCl (605 mg, 3.17 mmol) at 0° C. and the mixture stirred for 15 min. The mixture was then stirred at 25° C. under N₂ for 12.75 hrs. The crude was washed with water (10 mL) and extracted with DCM (2×20 mL). The organic layers were combined, dried, filtered and concentrated to give a residue which was purified by silica gel chromatography (PE-PE/EA=3/1) to give (1-(4-methoxybenzyl)-1,4,5,7-tetrahydropyrano[3,4-c]pyrazol-4-yl)methyl 4-methylbenzenesulfonate. TLC: PE/EA=1/1, Rf=0.5. LCMS: Rt=0.86 min, m/z=429.1 (M+H).

Step 6: To a solution of (1-(4-methoxybenzyl)-1,4,5,7-tetrahydropyrano[3,4-c]pyrazol-4-yl)methyl 4-methylbenzenesulfonate (500 mg, 1.11 mmol) in DMF (5 mL) was added 4-bromophenol (242 mg, 1.4 mmol) and Cs₂CO₃ (570 mg, 1.75 mmol) at 25° C. The mixture was stirred at 60° C. for 12 hrs. Water (5 mL) was added and the mixture was extracted with EtOAc (2×5 mL). The organic layers were combined and washed with brine, dried with anhydrous Na₂SO₄, filtered and concentrated in vacuum. The residue was purified by silica gel chromatography (PE-PE/EA=3/1) to give 4-((4-bromophenoxy)methyl)-1-(4-methoxybenzyl)-1,4,5,7-tetrahydropyrano[3,4-c]pyrazole. TLC: PE/EA=3/1, Rf=0.5. LCMS: Rt=0.93 min, m/z=429.0, 431.0 (M+H).

Step 7: To a solution of 4-((4-bromophenoxy)methyl)-1-(4-methoxybenzyl)-1,4,5,7-tetrahydropyrano[3,4-c]pyrazole (200 mg, 0.47 mmol) in dioxane (2 mL) was added TFA (1.36 g, 13.98 mmol) and trifluoromethanesulfonic acid (705 mg, 4.7 mmol) at 25° C. The mixture was stirred at 110° C. for 16 hrs. The mixture was washed with water (5 mL) and sat. NaHCO₃ solution (5 mL) and extracted with EtOAc (2×5 mL). The organic layers were combined, dried with anhydrous Na₂SO₄, filtered and concentrated in vacuum. The residue was purified by reversed-phase column (FA) to give 4-((4-bromophenoxy)methyl)-1,4,5,7-tetrahydropyrano[3,4-c]pyrazole. LCMS: Rt=0.788 min, m/z=309.1, 311.2 (M+H). ¹H NMR (400 MHz, CDCl₃) δ=7.52 (s, 1H), 7.39-7.36 (m, 2H), 6.80 (d, J=8.8 Hz, 2H), 4.10-3.87 (m, 4H), 3.81-3.68 (m, 2H), 3.32-3.23 (m, 1H).

Step 8: To a solution of 4-((4-bromophenoxy)methyl)-1,4,5,7-tetrahydropyrano[3,4-c]pyrazole (100 mg, 0.32 mol) in THF (1 mL) was added t-BuOK (72 mg, 0.64 mmol) at 0° C. The mixture was stirred at 0° C. for 0.5 hour. Iodomethane (72 mg, 0.64 mmol) was added to the mixture at 0° C. and the mixture was stirred at 25° C. for 15.5 hrs. The mixture was diluted with water (5 mL) and extracted with EtOAc (2×5 mL). The organic layers were combined, dried with anhydrous Na₂SO₄, filtered and concentrated in vacuum. The residue was purified by reverse phase column (FA) to give 4-((4-bromophenoxy)methyl)-1-methyl-1,4,5,7-tetrahydropyrano[3,4-c]pyrazole. LCMS: Rt=0.82 min, m/z=323.0, 325.0 (M+H).

Step 9: Pd(dtbpf)Cl₂ (10 mg, 0.02 mmol) was added to a solution of 4-((4-bromophenoxy)methyl)-1-methyl-1,4,5,7-tetrahydropyrano[3,4-c]pyrazole (63 mg, 0.19 mmol, 2-oxo-5-(4,4,5,5-tetramethyl-1,3,2-dioxaborolan-2-yl)-6-(trifluoromethyl)-1,2-dihydropyridine-3-carbonitrile (306 mg, 0.97 mmol) and K₃PO₄ (51 mg, 0.24 mmol) in dioxane/H₂O=4/1 (2 mL) at 25° C. and the mixture stirred at 75° C. under N₂ for 2 hrs. The reaction mixture was filtered and concentrated to dryness. The residue was purified by reversed-phase column (base) to give 5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carbonitrile. LCMS: Rt=0.79 min, MS m/z 431.0 (M+H).

### Example 290: 5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carboxamide (SFC Peak 3) (290)

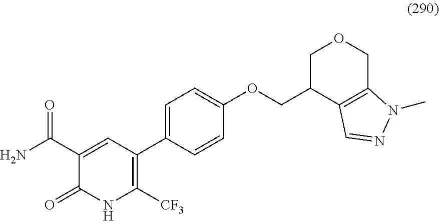 [SMILES: Cn1ncc2c1COCC2COc1ccc(-c2cc(C(N)=O)c(=O)[nH]c2C(F)(F)F)cc1]

5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carboxamide (SFC peak 3) (290) was isolated from the crude mixture of Example 289. ¹H NMR: (400 MHz, DMSO-d6), δ=8.34 (br, s, 1H), 8.18 (m, 1H), 7.94 (br, s, 1H), 7.43 (s, 1H), 7.25 (d, J=8.0 Hz, 2H), 7.05 (d, J=8.0 Hz, 2H), 4.79 (m, 2H), 4.08 (m, 2H), 4.02 (m, 2H), 3.82 (s, 3H), 3.16 (m, 1H). LCMS: Rt=0.73 min, m/z=449.3 (M+H). Chiral HPLC: Rt=3.24 min, ee value=93.3%.

### Example 291: 5-(4-((2-methyl-2,4,5,7-tetrahydropyrano[3,4-c]pyrazol-4-yl)methoxy)phenyl)-2-oxo-6-(trifluoromethyl)-1,2-dihydropyridine-3-carboxamide (SFC Peak 1)

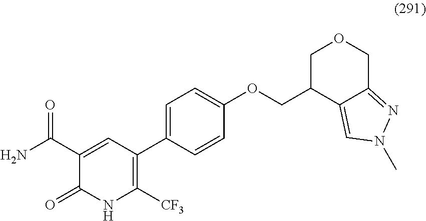 [SMILES: Cn1cc2c(n1)COCC2COc1ccc(-c2cc(C(N)=O)c(=O)[nH]c2C(F)(F)F)cc1]

5-(4-((2-methyl-2,4,5,7-tetrahydropyrano[3,4-c]pyrazol-4-yl)methoxy)phenyl)-2-oxo-6-(trifluoromethyl)-1,2-dihydropyridine-3-carboxamide (SFC peak 1) (291) was isolated from the crude mixture of Example 289. ¹H NMR: (400 MHz, DMSO-d6) δ=9.26 (br, s, 1H), 8.16 (m, 1H), 7.77 (br, s, 1H), 7.75 (s, 1H), 7.22 (d, J=8.0 Hz, 2H), 7.03 (d, J=8.0 Hz, 2H), 4.64 (m, 2H), 4.03 (m, 2H), 3.86 (m, 2H), 3.80 (s, 3H), 3.19 (m, 1H). LCMS: Rt=0.73 min, m/z=449.3 (M+H). Chiral HPLC: Rt=2.83 min, ee value=85.9%.

### Example 292: 5-(4-((2-methyl-2,4,5,7-tetrahydropyrano[3,4-c]pyrazol-4-yl)methoxy)phenyl)-2-oxo-6-(trifluoromethyl)-1,2-dihydropyridine-3-carboxamide (SFC Peak 2) (292)

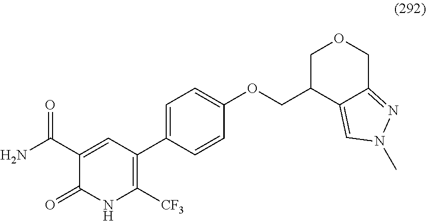 [SMILES: Cn1cc2c(n1)COCC2COc1ccc(-c2cc(C(N)=O)c(=O)[nH]c2C(F)(F)F)cc1]

5-(4-((2-methyl-2,4,5,7-tetrahydropyrano[3,4-c]pyrazol-4-yl)methoxy)phenyl)-2-oxo-6-(trifluoromethyl)-1,2-dihydropyridine-3-carboxamide (SFC peak 2) (292) was isolated from the crude mixture of Example 289. ¹H NMR: (400 MHz, DMSO-d6) δ=8.75 (br, s, 1H), 8.21 (br, s, 1H), 8.03 (br, s, 1H), 7.65 (s, 1H), 7.26 (d, J=8.0 Hz, 2H), 7.07 (d, J=8.0 Hz, 2H), 4.64 (m, 2H), 4.04 (m, 2H), 3.88 (m, 2H), 3.79 (s, 3H), 3.21 (m, 1H). LCMS: Rt=0.73 min, m/z=449.3 (M+H). Chiral HPLC: Rt=2.94 min, ee value=83.8%.

### Example 293: 5-(4-((1-isopropyl-1,4,5,7-tetrahydropyrano[3,4-c]pyrazol-4-yl)methoxy)phenyl)-2-oxo-6-(trifluoromethyl)-1,2-dihydropyridine-3-carboxamide (SFC Peak 3) (293)

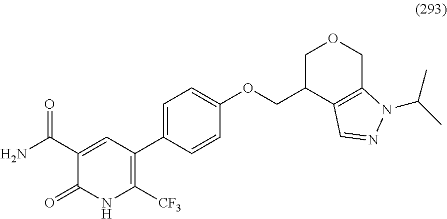 [SMILES: CC(C)n1ncc2c1COCC2COc1ccc(-c2cc(C(N)=O)c(=O)[nH]c2C(F)(F)F)cc1]

5-(4-((1-isopropyl-1,4,5,7-tetrahydropyrano[3,4-c]pyrazol-4-yl)methoxy)phenyl)-2-oxo-6-(trifluoromethyl)-1,2-dihydropyridine-3-carboxamide (SFC peak 3) (293) was obtained after SFC purification of the product obtained using the method described for the preparation of 5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carboxamide (SFC peak 4) (289), except 5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carbonitrile was replaced with 5-(4-((1-isopropyl-1,4,5,7-tetrahydropyrano[3,4-c]pyrazol-4-yl)methoxy)phenyl)-2-oxo-6-(trifluoromethyl)-1,2-dihydropyridine-3-carbonitrile. ¹H NMR: (400 MHz, DMSO-d6), δ=8.75-8.50 (m, 1H), 8.23 (s, 1H), 8.08 (br s, 1H), 7.45 (s, 1H), 7.26 (d, J=8.8 Hz, 2H), 7.07 (d, J=8.4 Hz, 2H), 4.78-4.73 (m, 2H), 4.34-4.24 (m, 1H), 4.14-4.06 (m, 1H), 4.00 (s, 1H), 3.83 (m, 2H), 3.21-3.09 (m, 1H), 1.36-1.32 (m, 6H). LCMS: Rt=0.75 min, m/z=477.3 (M+H). Chiral HPLC: Rt=2.46 min, ee value=100%.

5-(4-((1-isopropyl-1,4,5,7-tetrahydropyrano[3,4-c]pyrazol-4-yl)methoxy)phenyl)-2-oxo-6-(trifluoromethyl)-1,2-dihydropyridine-3-carbonitrile was obtained using the procedure for the preparation of 5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carbonitrile, except iodomethane was replaced with 2-iodopropane.

### Example 294: 5-(4-((1-isopropyl-1,4,5,7-tetrahydropyrano[3,4-c]pyrazol-4-yl)methoxy)phenyl)-2-oxo-6-(trifluoromethyl)-1,2-dihydropyridine-3-carboxamide (SFC Peak 4) (294)

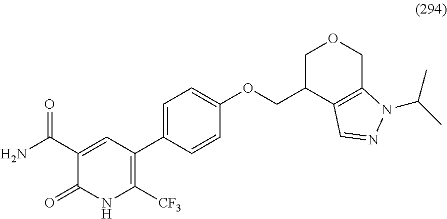 [SMILES: CC(C)n1ncc2c1COCC2COc1ccc(-c2cc(C(N)=O)c(=O)[nH]c2C(F)(F)F)cc1]

5-(4-((1-isopropyl-1,4,5,7-tetrahydropyrano[3,4-c]pyrazol-4-yl)methoxy)phenyl)-2-oxo-6-(trifluoromethyl)-1,2-dihydropyridine-3-carboxamide (SFC peak 4) (294) was isolated from the crude mixture of Example 293. ¹H NMR: (400 MHz, DMSO-d6), δ=8.71 (brs, 1H), 8.21 (br s, 1H), 8.02 (br s, 1H), 7.45 (s, 1H), 7.25 (d, J=8.0 Hz, 2H), 7.06 (d, J=7.6 Hz, 2H), 4.86-4.67 (m, 2H), 4.34-4.22 (m, 1H), 4.15-4.06 (m, 1H), 4.05-3.95 (m, 1H), 3.90-3.76 (m, 2H), 3.21-3.08 (m, 1H), 1.36-1.32 (m, 6H). LCMS: Rt=0.76 min, m/z=477.3 (M+H). Chiral HPLC: Rt=3.22 min, ee value=89.5%.

### Example 295: 5-(4-((2-isopropyl-2,4,5,7-tetrahydropyrano[3,4-c]pyrazol-4-yl)methoxy)phenyl)-2-oxo-6-(trifluoromethyl)-1,2-dihydropyridine-3-carboxamide (SFC Peak 1) (295)

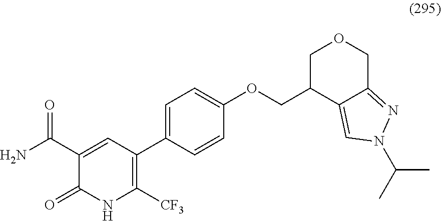 [SMILES: CC(C)n1cc2c(n1)COCC2COc1ccc(-c2cc(C(N)=O)c(=O)[nH]c2C(F)(F)F)cc1]

5-(4-((2-isopropyl-2,4,5,7-tetrahydropyrano[3,4-c]pyrazol-4-yl)methoxy)phenyl)-2-oxo-6-(trifluoromethyl)-1,2-dihydropyridine-3-carboxamide (SFC peak 1) (295) was isolated from the product in Example 293. ¹H NMR: (400 MHz, DMSO-d6), δ=9.78-9.78 (m, 1H), 8.49 (d, J=15.6 Hz, 2H), 8.20 (s, 1H), 7.96 (s, 1H), 7.72 (d, J=8.2 Hz, 1H), 7.58 (m, 1H), 4.57 (m, 1H), 4.13 (m, 1H), 3.98-3.89 (m, 2H), 3.21-3.05 (m, 2H), 2.80-2.58 (m, 1H), 1.70 (m, 4H). LCMS: Rt=0.76 min, m/z=477.3 (M+H). Chiral HPLC: Rt=2.90 min, ee value=100%.

### Example 296: 5-(4-((2-isopropyl-2,4,5,7-tetrahydropyrano[3,4-c]pyrazol-4-yl)methoxy)phenyl)-2-oxo-6-(trifluoromethyl)-1,2-dihydropyridine-3-carboxamide (SFC Peak 2) (296)

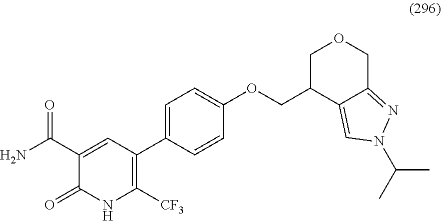 [SMILES: CC(C)n1cc2c(n1)COCC2COc1ccc(-c2cc(C(N)=O)c(=O)[nH]c2C(F)(F)F)cc1]

5-(4-((2-isopropyl-2,4,5,7-tetrahydropyrano[3,4-c]pyrazol-4-yl)methoxy)phenyl)-2-oxo-6-(trifluoromethyl)-1,2-dihydropyridine-3-carboxamide (SFC peak 2) (296) as isolated from the product in Example 293. ¹H NMR: (DMSO, 400 MHz), δ: 9.22 (br s, 1H), 8.11 (s, 1H), 7.76 (br s, 1H), 7.72 (s, 1H), 7.22 (d, J=8.8 Hz, 2H), 7.04 (d, J=8.8 Hz, 2H), 4.61 (s, 2H), 3.89-3.88 (m, 1H), 3.86-3.85 (m, 2H), 3.85-3.83 (m, 2H), 3.82-3.80 (m, 1H), 1.40-1.37 (m, 6H). LCMS: Rt=0.76 min, m/z=477.4 (M+H). Chiral HPLC: Rt=3.13 min, ee value=83%.

### Example 297: 5-(4-(chroman-2-ylmethoxy)phenyl)-2-oxo-6-(trifluoromethyl)-1,2-dihydropyridine-3-carboxamide (297)

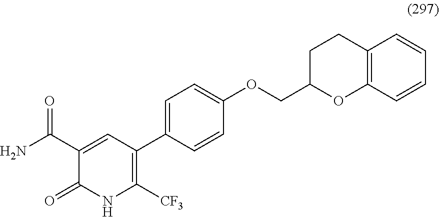 [SMILES: NC(=O)c1cc(-c2ccc(OCC3CCc4ccccc4O3)cc2)c(C(F)(F)F)[nH]c1=O]

5-(4-(chroman-2-ylmethoxy)phenyl)-2-oxo-6-(trifluoromethyl)-1,2-dihydropyridine-3-carboxamide (297) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with chroman-2-ylmethanol. ¹H NMR: (400 MHz, DMSO-d6) δ=10.41 (s, 1H), 8.27 (s, 1H), 7.87 (d, J=0.9 Hz, 1H), 7.20-6.96 (m, 6H), 6.82 (d, J=13.1 Hz, 2H), 4.41 (d, J=2.9 Hz, 1H), 4.30-4.17 (m, 2H), 2.80 (s, 2H), 2.14-1.76 (m, 2H). LCMS: Rt=0.90 min, m/z=445.1 (M+H).

### Example 298: tert-butyl 3-((4-(5-carbamoyl-6-oxo-2-(trifluoromethyl)-1,6-dihydropyridin-3-yl)phenoxy)methyl)azetidine-1-carboxylate (298)

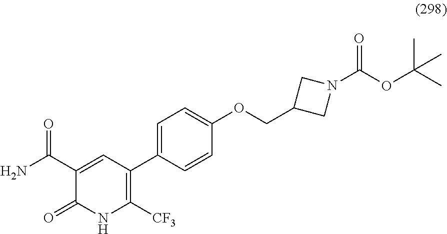 [SMILES: CC(C)(C)OC(=O)N1CC(COc2ccc(-c3cc(C(N)=O)c(=O)[nH]c3C(F)(F)F)cc2)C1]

tert-butyl 3-((4-(5-carbamoyl-6-oxo-2-(trifluoromethyl)-1,6-dihydropyridin-3-yl)phenoxy)methyl)azetidine-1-carboxylate (298) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with tert-butyl 3-(hydroxymethyl)azetidine-1-carboxylate. ¹H NMR (400 MHz, DMSO-d6) δ=10.88 (br s, 1H), 7.77 (s, 1H), 7.13 (d, J=8.4 Hz, 2H), 6.96 (br d, J=7.6 Hz, 1H), 6.93 (d, J=8.8 Hz, 2H), 4.13 (d, J=6.8 Hz, 2H), 3.97 (br s, 5 2H), 3.69 (br s, 2H), 2.67 (m, 1H), 1.39 (s, 9H). LCMS: Rt=0.83 min, m/z=468.1 (M+H).

### Example 299: Tert-butyl ((1r,3r)-3-((4-(5-carbamoyl-6-oxo-2-(trifluoromethyl)-1,6-dihydropyridin-3-yl)phenoxy)methylcyclobutyl)carbamate (299)

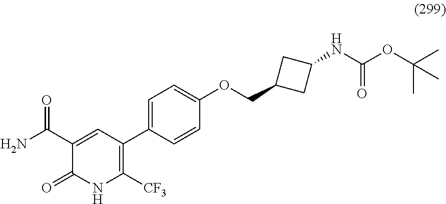 [SMILES: CC(C)(C)OC(=O)N[C@H]1C[C@H](COc2ccc(-c3cc(C(N)=O)c(=O)[nH]c3C(F)(F)F)cc2)C1]

tert-butyl ((1r,3r)-3-((4-(5-carbamoyl-6-oxo-2-(trifluoromethyl)-1,6-dihydropyridin-3-yl)phenoxy)methyl)cyclobutyl)carbamate (299) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with tert-butyl ((1r,3r)-3-(hydroxymethyl)cyclobutyl)carbamate. ¹H NMR (400 MHz, DMSO-d6) δ=8.10 (br s, 1H), 7.81 (m, 1H), 7.21 (br d, J=8.4 Hz, 2H), 7.14 (br d, J=7.8 Hz, 1H), 6.97 (d, J=8.4 Hz, 2H), 3.93 (br d, J=5.0 Hz, 2H), 3.88 (m, 1H), 2.32 (m, 2H), 2.09 (s, 1H), 1.75 (br d, J=7.4 Hz, 2H), 1.37 (s, 9H). LCMS: Rt=0.78 min, m/z=482.4 (M+H).

### Example 300: (S)-5-(4-((5,5-dimethyltetrahydrofuran-2-yl)methoxy)phenyl)-2-oxo-6-(trifluoromethyl)-1,2-dihydropyridine-3-carboxamide (300)

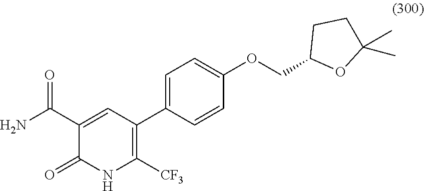 [SMILES: CC1(C)CC[C@@H](COc2ccc(-c3cc(C(N)=O)c(=O)[nH]c3C(F)(F)F)cc2)O1]

(S)-5-(4-((5,5-dimethyltetrahydrofuran-2-yl)methoxy)phenyl)-2-oxo-6-(trifluoromethyl)-1,2-dihydropyridine-3-carboxamide (300) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5,5-dimethyltetrahydrofuran-2-yl)methanol. ¹H NMR (400 MHz, DMSO-d6) δ=8.53 (br s, 1H), 8.27 (s, 1H), 8.15 (br s, 1H), 7.26 (br d, J=8.4 Hz, 2H), 7.03 (d, J=8.7 Hz, 2H), 4.10-3.79 (m, 4H), 3.77-3.61 (m, 2H), 3.42-3.23 (m, 2H), 3.08 (t, J=11.0 Hz, 1H), 2.54-2.47 (m, 5H), 1.03 (d, J=6.1 Hz, 3H). LCMS: Rt=0.73 min, m/z=413.1 (M+H). Chiral HPLC: Rt=2.11 min, de value=99.1%.

### Example 301: Tert-butyl 6-((4-(5-carbamoyl-6-oxo-2-(trifluoromethyl)-1,6-dihydropyridin-3-yl)phenoxy)methyl)-3-azabicyclo[3.1.0]hexane-3-carboxylate (301)

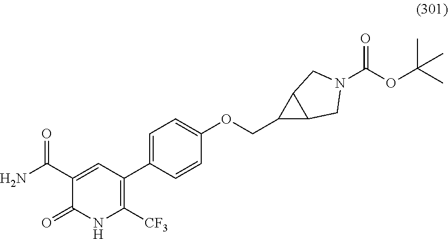 [SMILES: CC(C)(C)OC(=O)N1CC2C(COc3ccc(-c4cc(C(N)=O)c(=O)[nH]c4C(F)(F)F)cc3)C2C1]

Tert-butyl 6-((4-(5-carbamoyl-6-oxo-2-(trifluoromethyl)-1,6-dihydropyridin-3-yl)phenoxy)methyl)-3-azabicyclo[3.1.0]hexane-3-carboxylate (301) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with tert-butyl 6-(hydroxymethyl)-3-azabicyclo[3.1.0]hexane-3-carboxylate. ¹H NMR (400 MHz, DMSO-d6) δ=9.53-8.93 (m, 1H), 8.10 (s, 1H), 7.76 (s, 1H), 7.20 (d, J=8.0 Hz, 2H), 6.97 (d, J=8.0 Hz, 2H), 4.09-3.97 (m, 1H), 3.91-3.78 (m, 1H), 3.44 (s, 5H), 1.81 (d, J=8.0 Hz, 2H), 1.43 (m, 1H), 1.29 (s, 9H). LCMS: Rt=0.80 min, m/z=438.1 (M-Bu).

### Example 302: Tert-butyl 3-((4-(5-carbamoyl-6-oxo-2-(trifluoromethyl)-1,6-dihydropyridin-3-yl)phenoxy)methyl)pyrrolidine-1-carboxylate (302)

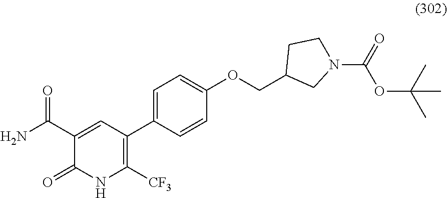 [SMILES: CC(C)(C)OC(=O)N1CCC(COc2ccc(-c3cc(C(N)=O)c(=O)[nH]c3C(F)(F)F)cc2)C1]

Tert-butyl 3-((4-(5-carbamoyl-6-oxo-2-(trifluoromethyl)-1,6-dihydropyridin-3-yl)phenoxy)methyl)pyrrolidine-1-carboxylate (302) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with tert-butyl 3-(hydroxymethyl)pyrrolidine-1-carboxylate. ¹H NMR (400 MHz, MeOD-d4) δ=8.30 (s, 1H), 7.25 (d, J=8.4 Hz, 2H), 7.01 (d, J=8.7 Hz, 2H), 4.14-3.92 (m, 2H), 3.65-3.46 (m, 2H), 3.26-3.20 (m, 1H), 3.28-3.16 (m, 1H), 2.72 (br d, J=5.7 Hz, 1H), 2.12 (br s, 1H), 1.85 (br d, J=8.4 Hz, 1H), 1.47 (s, 9H); 19F NMR: (377 MHz, MeOD-d4) δ=−62.74 (br s, 3F). LCMS: Rt=0.92 min, m/z=504.3 (M+Na).

### Example 303: Tert-butyl 3-((4-(5-carbamoyl-6-oxo-2-(trifluoromethyl)-1,6-dihydropyridin-3-yl)phenoxy)methyl)-4-methylpyrrolidine-1-carboxylate (303)

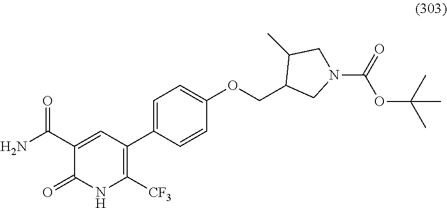 [SMILES: CC1CN(C(=O)OC(C)(C)C)CC1COc1ccc(-c2cc(C(N)=O)c(=O)[nH]c2C(F)(F)F)cc1]

Tert-butyl 3-((4-(5-carbamoyl-6-oxo-2-(trifluoromethyl)-1,6-dihydropyridin-3-yl)phenoxy)methyl)-4-methylpyrrolidine-1-carboxylate (303) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with tert-butyl 3-(hydroxymethyl)-4-methylpyrrolidine-1-carboxylate. ¹H NMR (400 MHz, DMSO-d6) δ=8.11 (br s, 1H), 7.77 (br s, 1H), 7.22 (br d, J=8.4 Hz, 2H), 7.00 (br d, J=8.4 Hz, 2H), 4.10 (brdd, J=4.6, 9.3 Hz, 1H), 4.03-3.91 (m, 1H), 3.55 (br dd, J=7.3, 10.4 Hz, 2H), 3.18-3.05 (m, 1H), 2.85 (q, J=10.2 Hz, 1H), 2.28-2.02 (m, 3H), 1.40 (s, 10H), 1.12-1.02 (m, 3H). 19F NMR (376 MHz, DMSO-d6) δ=−59.31-−61.74 (m, 3F). LCMS: Rt=0.81 min, m/z=440.3 (M-Bu).

### Example 304: 5-(4-((1-benzyl-3-cyanopyrrolidin-3-yl)methoxy)phenyl)-2-oxo-6-(trifluoromethyl)-1,2-dihydropyridine-3-carboxamide (304)

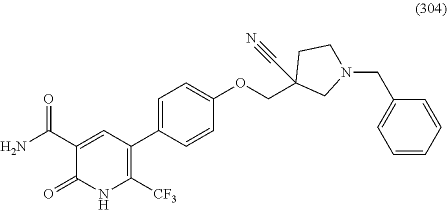 [SMILES: N#CC1(COc2ccc(-c3cc(C(N)=O)c(=O)[nH]c3C(F)(F)F)cc2)CCN(Cc2ccccc2)C1]

5-(4-((1-benzyl-3-cyanopyrrolidin-3-yl)methoxy)phenyl)-2-oxo-6-(trifluoromethyl)-1,2-dihydropyridine-3-carboxamide (304) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with 1-benzyl-3-(hydroxymethyl)pyrrolidine-3-carbonitrile. ¹H NMR (400 MHz, DMSO-d6) δ=8.10 (br s, 1H), 7.72 (br s, 1H), 7.34 (m, 5H), 7.26 (m, 2H), 7.32 (m, 2H), 4.21 (m, 2H), 3.67 (m, 2H), 2.96 (m, 1H), 2.75 (m, 2H), 2.53 (m, 1H), 2.33 (m, 1H), 2.08 (m, 1H). LCMS: Rt=0.80 min, m/z=497.3 (M+H).

### Example 305: Tert-butyl (2S,4R)-2-((4-(5-carbamoyl-6-oxo-2-(trifluoromethyl)-1,6-dihydropyridin-3-yl)phenoxy)methyl)-4-fluoropyrrolidine-1-carboxylate (305)

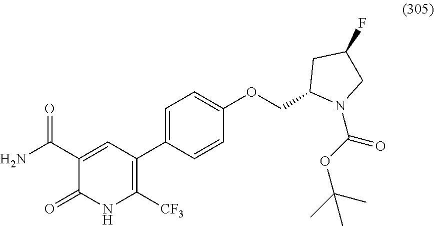 [SMILES: CC(C)(C)OC(=O)N1C[C@H](F)C[C@H]1COc1ccc(-c2cc(C(N)=O)c(=O)[nH]c2C(F)(F)F)cc1]

Tert-butyl (2S,4R)-2-((4-(5-carbamoyl-6-oxo-2-(trifluoromethyl)-1,6-dihydropyridin-3-yl)phenoxy)methyl)-4-fluoropyrrolidine-1-carboxylate (305) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with tert-butyl 4-fluoro-2-(hydroxymethyl)pyrrolidine-1-carboxylate. ¹H NMR (400 MHz, DMSO-d6) δ=9.27 (brs, 1H), 8.13 (s, 1H), 7.73 (br s, 1H), 7.22 (d, J=8.4 Hz, 2H), 7.01 (d, J=8.4 Hz, 2H), 5.25 (s, 1H), 4.22 (m, 3H), 3.74 (s, 1H), 3.40 (m, 2H), 2.34 (m, 1H), 1.40 (s, 9H). LCMS: Rt=1.02 min, m/z=522.2 (M+Na). Chiral HPLC: Rt=1.42 min, de value=100%.

### Example 306: tert-butyl (2S,4S)-2-((4-(5-carbamoyl-6-oxo-2-(trifluoromethyl)-1,6-dihydropyridin-3-yl)phenoxy)methyl)-4-fluoropyrrolidine-1-carboxylate (306)

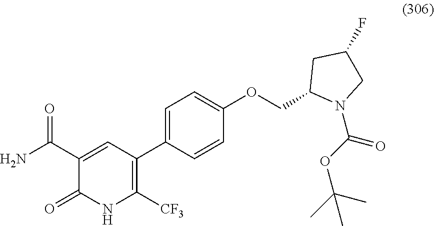 [SMILES: CC(C)(C)OC(=O)N1C[C@@H](F)C[C@H]1COc1ccc(-c2cc(C(N)=O)c(=O)[nH]c2C(F)(F)F)cc1]

tert-butyl (2S,4S)-2-((4-(5-carbamoyl-6-oxo-2-(trifluoromethyl)-1,6-dihydropyridin-3-yl)phenoxy)methyl)-4-fluoropyrrolidine-1-carboxylate (306) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with tert-butyl 4-fluoro-2-(hydroxymethyl)pyrrolidine-1-carboxylate. ¹H NMR (400 MHz, DMSO-d6) δ=9.60 (m, 1H), 8.16 (m, 1H), 8.02 (m, 1H), 7.70-7.51 (m, 1H), 7.20 (d, J=8.4 Hz, 2H), 7.02 (br d, J=8.8 Hz, 2H), 5.52-5.16 (m, 1H), 4.36-4.10 (m, 2H), 3.90-3.74 (m, 1H), 3.71-3.44 (m, 3H), 2.30 (m, 2H), 1.42 (s, 9H). LCMS: Rt=0.88 min, m/z=444.1 (M+H). Chiral HPLC: Rt=2.16 min, de value=100%.

### Example 307: 5-(4-((3-isopropyl-2-oxooxazolidin-5-yl)methoxy)phenyl)-2-oxo-6-(trifluoromethyl)-1,2-dihydropyridine-3-carboxamide (307)

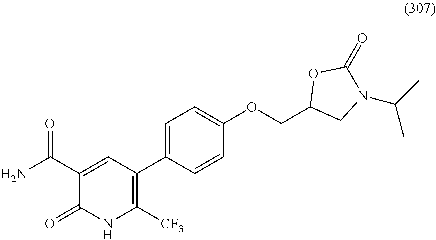 [SMILES: CC(C)N1CC(COc2ccc(-c3cc(C(N)=O)c(=O)[nH]c3C(F)(F)F)cc2)OC1=O]

5-(4-((3-isopropyl-2-oxooxazolidin-5-yl)methoxy)phenyl)-2-oxo-6-(trifluoromethyl)-1,2-dihydropyridine-3-carboxamide (307) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-((4-bromophenoxy)methyl)-3-isopropyloxazolidin-2-one. ¹H NMR: (400 MHz, DMSO-d6) δ=8.76 (s, 1H), 8.21 (s, 1H), 7.99 (s, 1H), 7.27 (d, J=8.0 Hz, 2H), 7.03 (d, J=8.0 Hz, 2H), 4.87 (s, 1H), 4.29-4.10 (m, 2H), 3.99-3.84 (m, 1H), 3.65 (t, J=8.0 Hz, 1H), 3.39 (d, J=4.0 Hz, 1H), 1.14 (d, J=4.0 Hz, 6H). LCMS: Rt=0.68 min, m/z=440.1 (M+H).

### 5-((4-bromophenoxy)methyl)-3-isopropyloxazolidin-2-one was Obtained Using the Following Procedure

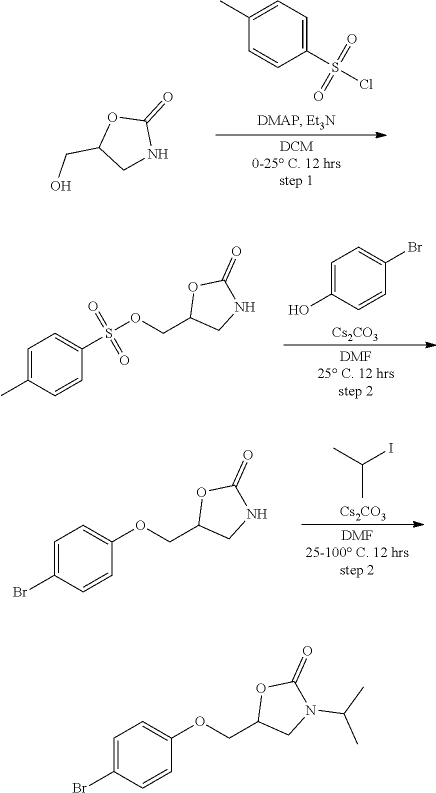

Step 1: Tosyl chloride (2.44 g, 12.81 mmol) was added to a solution of 5-(hydroxymethyl)oxazolidin-2-one (1.0 g, 8.54 mmol), DMAP (522 mg, 4.27 mmol) and Et₃N (1.73 g, 17.08 mmol) in DCM (10 mL) at 0° C. and the mixture stirred at 25° C. for 12 hrs. The reaction mixture was poured into water (20 mL) and extracted with EtOAC (2×20 mL). The organic layers were combined, dried over anhydrous Na₂SO₄, filtered and concentrated to give crude product which was purified by silica gel column (PE/EA=10/1) to give (2-oxooxazolidin-5-yl)methyl 4-methylbenzenesulfonate. LCMS: Rt=0.64 min, m/z=272.1 (M+H). ¹H NMR (400 MHz, DMSO-d6) δ=7.80 (d, J=8.0 Hz, 2H), 7.55 (s, 1H), 7.50 (d, J=8.0 Hz, 2H), 4.79-4.71 (m, 1H), 4.23-4.18 (m, 1H), 4.16-4.10 (m, 1H), 3.50 (t, J=8.0 Hz, 1H), 3.14 (m, 1H), 2.43 (s, 3H).

Step 2: To a solution of 4-bromophenol (1.47 g, 8.48 mmol) in DMF (20 mL) was added Cs₂CO₃ (5.52 g, 16.96 mmol) followed by (2-oxooxazolidin-5-yl)methyl 4-methylbenzenesulfonate (2.3 g, 8.48 mmol) at 25° C. The mixture was then stirred at 25° C. for 12 hrs. The reaction mixture was poured into water (50 mL) and extracted with EtOAc (2×50 mL). The organic layers were combined, washed with brine (2×50 mL), dried over anhydrous Na₂SO₄, filtered and concentrated to give crude product. The crude product was stirred with PE/EA (10/1) and precipitate formed. The precipitate was filtered and dried under vacuo to give 5-((4-bromophenoxy)methyl)oxazolidin-2-one. LCMS: Rt=0.70 min, m/z=272, 274 (M+H). ¹H NMR (400 MHz, DMSO-d6) δ=7.60 (s, 1H), 7.47 (d, J=8.8 Hz, 2H), 6.95 (d, J=8.8 Hz, 2H), 4.89 (m, 1H), 4.19-4.06 (m, 2H), 3.61 (t, J=9.0 Hz, 1H), 3.32-3.28 (m, 1H).

Step 3: Cs₂CO₃ (1.44 g, 4.41 mmol) and 2-iodopropane (750 mg, 4.41 mmol) were added to a solution of 5-((4-bromophenoxy)methyl)oxazolidin-2-one (400 mg, 1.47 mmol) in DMF (10 mL) at 25° C. and the mixture stirred at 100° C. for 12 hrs. The reaction mixture was poured into water (20 mL) and extracted with EtOAc (2×10 ml). The organic layers were combined, washed with brine (2×10 mL), dried over anhydrous Na₂SO₄, filtered and concentrated to give crude product which was purified by silica gel column (PE/EA=5/1) to give 5-((4-bromophenoxy)methyl)-3-isopropyloxazolidin-2-one. LCMS: Rt=0.78 min, m/z=314, 316 (M+H). ¹H NMR (400 MHz, DMSO-d6) δ=7.46 (d, J=8.0 Hz, 2H), 6.93 (d, J=8.0 Hz, 2H), 4.90-4.79 (m, 1H), 4.23-4.14 (m, 1H), 4.12-4.06 (m, 1H), 3.89 (m, 1H), 3.62 (t, J=8.0 Hz, 1H), 1.12 (d, J=4.0 Hz, 6H).

### Example 308: 5-(4-((3-(cyclopropylmethyl)-2-oxooxazolidin-5-yl)methoxy)phenyl)-2-oxo-6-(trifluoromethyl)-1,2-dihydropyridine-3-carboxamide (308)

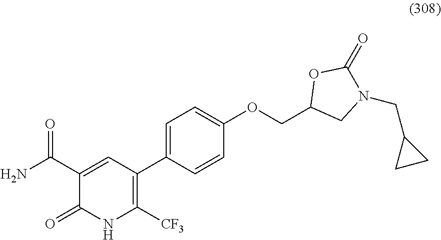 [SMILES: NC(=O)c1cc(-c2ccc(OCC3CN(CC4CC4)C(=O)O3)cc2)c(C(F)(F)F)[nH]c1=O]

5-(4-((3-(cyclopropylmethyl)-2-oxooxazolidin-5-yl)methoxy)phenyl)-2-oxo-6-(trifluoromethyl)-1,2-dihydropyridine-3-carboxamide (308) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 5-((4-bromophenoxy)methyl)-3-(cyclopropylmethyl)oxazolidin-2-one. ¹H NMR (400 MHz, DMSO-d6) δ=13.63 (s, 1H), 13.87-13.42 (m, 1H), 8.21 (s, 1H), 8.04 (s, 1H), 7.27 (d, J=8.4 Hz, 2H), 7.04 (d, J=8.4 Hz, 2H), 4.91 (m, 1H), 4.33-4.12 (m, 2H), 3.80 (m, 1H), 3.52 (m, 1H), 3.06 (m, 2H), 1.06-0.90 (m, 1H), 0.56-0.43 (m, 2H), 0.33-0.15 (m, 2H). LCMS: Rt=0.75 min, m/z=452.0 (M+H).

5-((4-bromophenoxy)methyl)-3-(cyclopropylmethyl)oxazolidin-2-one was obtained using the method similar to the preparation of 5-((4-bromophenoxy)methyl)-3-isopropyloxazolidin-2-one, except 2-iodopropane was replaced with bromomethyl)cyclopropane.

### Example 309: 5-(4-((3-ethyl-2-oxooxazolidin-4-yl)methoxy)phenyl)-2-oxo-6-(trifluoromethyl)-1,2-dihydropyridine-3-carboxamide (309)

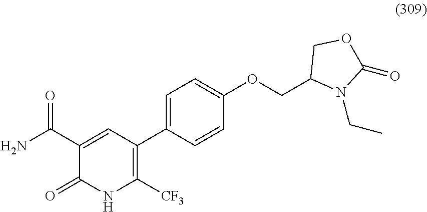 [SMILES: CCN1C(=O)OCC1COc1ccc(-c2cc(C(N)=O)c(=O)[nH]c2C(F)(F)F)cc1]

5-(4-((3-ethyl-2-oxooxazolidin-4-yl)methoxy)phenyl)-2-oxo-6-(trifluoromethyl)-1,2-dihydropyridine-3-carboxamide (309) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 4-((4-bromophenoxy)methyl)-3-ethyloxazolidin-2-one. ¹H NMR (400 MHz, DMSO-d6) δ=9.02 (m, 1H), 8.17 (d, J=6.0 Hz, 1H), 7.88 (br s, 1H), 7.26 (d, J=8.4 Hz, 2H), 7.04 (d, J=8.8 Hz, 2H), 4.42 (m, 1H), 4.21 (m, 2H), 4.15 (m, 2H), 2.52 (m, 2H), 1.11 (m, 3H). LCMS: Rt=0.65 min, m/z=426.1 (M+H).

4-((4-bromophenoxy)methyl)-3-ethyloxazolidin-2-one was obtained using the method similar to the preparation of 5-((4-bromophenoxy)methyl)-3-isopropyloxazolidin-2-one, except 5-(hydroxymethyl)oxazolidin-2-one was replaced with 4-(hydroxymethyl)oxazolidin-2-one, and 2-iodopropane was replaced with iodoethane. LCMS: Rt=0.79 min, m/z=299.9, 301.8 (M+H).

### Example 310: 5-(4-((3-isopropyl-2-oxooxazolidin-4-yl)methoxy)phenyl)-2-oxo-6-(trifluoromethyl)-1,2-dihydropyridine-3-carboxamide (310)

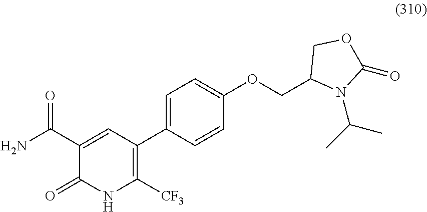 [SMILES: CC(C)N1C(=O)OCC1COc1ccc(-c2cc(C(N)=O)c(=O)[nH]c2C(F)(F)F)cc1]

5-(4-((3-isopropyl-2-oxooxazolidin-4-yl)methoxy)phenyl)-2-oxo-6-(trifluoromethyl)-1,2-dihydropyridine-3-carboxamide (310) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 4-((4-bromophenoxy)methyl)-3-isopropyloxazolidin-2-one. ¹H NMR (400 MHz, DMSO-d6) δ: 10.23 (br s, 1H), 7.91 (s, 1H), 7.31 (m, 1H), 7.19 (d, J=8.4 Hz, 2H), 6.98 (d, J=8.4 Hz, 2H), 4.38 (m, 2H), 4.18 (m, 2H), 4.16-4.09 (m, 3H), 3.83 (m, 2H), 1.27 (d, J=6.8 Hz, 2H), 1.19 (d, J=6.8 Hz, 2H). LCMS: Rt=0.67 min, m/z=440.0 (M+H).

4-((4-bromophenoxy)methyl)-3-isopropyloxazolidin-2-one was obtained using the method similar to the preparation of 4-((4-bromophenoxy)methyl)-3-ethyloxazolidin-2-one, except iodoethane was replaced with 2-iodopropane.

### Example 311: 5-(4-((3-(cyclopropylmethyl)-2-oxooxazolidin-4-yl)methoxy)phenyl)-2-oxo-6-(trifluoromethyl)-1,2-dihydropyridine-3-carboxamide (311)

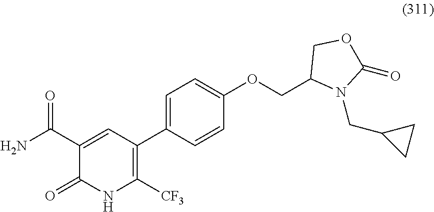 [SMILES: NC(=O)c1cc(-c2ccc(OCC3COC(=O)N3CC3CC3)cc2)c(C(F)(F)F)[nH]c1=O]

5-(4-((3-(cyclopropylmethyl)-2-oxooxazolidin-4-yl)methoxy)phenyl)-2-oxo-6-(trifluoromethyl)-1,2-dihydropyridine-3-carboxamide (311) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 4-((4-bromophenoxy)methyl)-3-(cyclopropylmethyl)oxazolidin-2-one. ¹H NMR (400 MHz, DMSO-d6) δ=10.34 (br s, 1H), 7.66 (s, 1H), 6.98 (d, J=8.4 Hz, 3H), 6.95 (m, 1H), 6.78 (d, J=8.4 Hz, 2H), 4.30 (m, 1H), 4.25 (m, 1H), 4.03 (m, 4H), 2.83 (m, 1H), 0.83 (m, 1H), 0.31 (m, 1H), 0.23 (m, 1H), 0.15 (m, 1H), 0.01 (m, 1H). LCMS: Rt=0.69 min, m/z=452.1 (M+H).

4-((4-bromophenoxy)methyl)-3-(cyclopropylmethyl)oxazolidin-2-one was obtained using the method similar to the preparation of 4-((4-bromophenoxy)methyl)-3-ethyloxazolidin-2-one, except iodoethane was replaced with (bromomethyl)cyclopropane.

### Example 312: 5-(4-((7-oxabicyclo[2.2.1]heptan-2-yl)methoxy)phenyl)-2-oxo-6-(trifluoromethyl)-1,2-dihydropyridine-3-carboxamide (Cis Isomer, SFC Peak 1) (312)

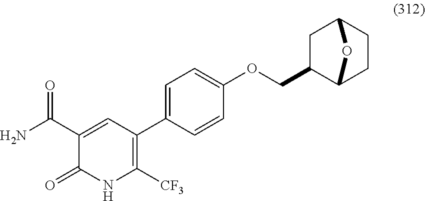

5-(4-((7-oxabicyclo[2.2.1]heptan-2-yl)methoxy)phenyl)-2-oxo-6-(trifluoromethyl)-1,2-dihydropyridine-3-carboxamide (cis isomer, SFC peak 1) (312) was obtained after SFC purification of the product made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 2-((4-bromophenoxy)methyl)-7-oxabicyclo[2.2.1]heptane. ¹H NMR (400 MHz, DMSO-d6) δ=8.60 (br s, 1H), 8.24 (s, 1H), 8.10 (br s, 1H), 7.25 (d, J=8.6 Hz, 2H), 7.01 (d, J=8.6 Hz, 2H), 4.55-4.38 (m, 2H), 3.79-3.63 (m, 2H), 2.27-2.16 (m, 1H), 1.71-1.41 (m, 5H), 1.29-1.18 (m, 1H). 19F NMR (376 MHz, DMSO-d6) δ=−60.40 (br s, 3F). LCMS: Rt=0.75 min, m/z=409.3 (M+H). Chiral HPLC: Rt=3.455 min, ee value=98.5%.

### Example 313: 5-(4-((7-oxabicyclo[2.2.1]heptan-2-yl)methoxy)phenyl)-2-oxo-6-(trifluoromethyl)-1,2-dihydropyridine-3-carboxamide (Cis Isomer, SFC Peak 2) (313)

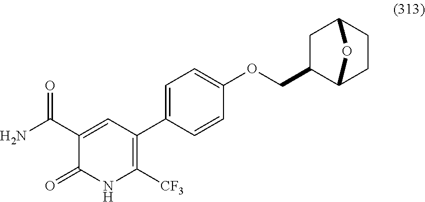

5-(4-((7-oxabicyclo[2.2.1]heptan-2-yl)methoxy)phenyl)-2-oxo-6-(trifluoromethyl)-1,2-dihydropyridine-3-carboxamide (cis isomer, SFC peak 2) (313) was obtained after SFC purification of the product made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 2-((4-bromophenoxy)methyl)-7-oxabicyclo[2.2.1]heptane. ¹H NMR (400 MHz, DMSO-d6) δ=8.65 (br s, 1H), 8.23 (s, 1H), 8.07 (br s, 1H), 7.24 (d, J=8.6 Hz, 2H), 7.01 (d, J=8.8 Hz, 2H), 4.57-4.38 (m, 2H), 3.78-3.65 (m, 2H), 2.27-2.15 (m, 1H), 1.70-1.41 (m, 5H), 1.28-1.18 (m, 1H). 19F NMR (376 MHz, DMSO-d6) δ=−60.39 (br s, 3F). LCMS: Rt=0.75 min, m/z 409.3 (M+H). Chiral HPLC: Rt=4.41 min, ee value=100%.

### Example 314: 5-(4-((7-oxabicyclo[2.2.1]heptan-2-yl)methoxy)phenyl)-2-oxo-6-(trifluoromethyl)-1,2-dihydropyridine-3-carboxamide (Trans Isomer, SFC Peak 1) (314)

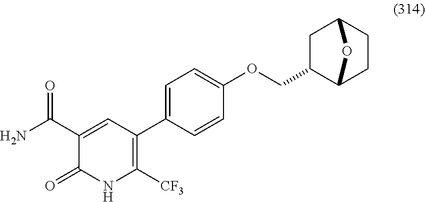

5-(4-((7-oxabicyclo[2.2.1]heptan-2-yl)methoxy)phenyl)-2-oxo-6-(trifluoromethyl)-1,2-dihydropyridine-3-carboxamide (trans isomer, SFC peak 1) (314) was obtained after SFC purification of the product made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 2-((4-bromophenoxy)methyl)-7-oxabicyclo[2.2.1]heptane. ¹H NMR (400 MHz, DMSO-d6) δ=8.18 (s, 1H), 7.21 (d, J=8.0 Hz, 2H), 6.97 (d, J=8.0 Hz, 2H), 4.65 (t, J=4.0 Hz, 1H), 4.60-4.54 (m, 1H), 4.18-4.14 (m, 1H), 3.95 (t, J=8.0 Hz, 1H), 2.67-2.54 (m, 1H), 2.04-1.95 (m, 1H), 1.93-1.84 (m, 1H), 1.79-1.59 (m, 2H), 1.56-1.48 (m, 1H), 1.21-1.17 (m, 1H). LCMS: Rt=0.73 min, m/z=409 (M+H). Chiral HPLC: Rt=2.46 min, ee value=100%.

### Example 315: 5-(4-((7-oxabicyclo[2.2.1]heptan-2-yl)methoxy)phenyl)-2-oxo-6-(trifluoromethyl)-1,2-dihydropyridine-3-carboxamide (Trans Isomer, SFC Peak 2) (315)

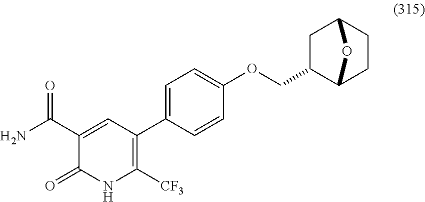

5-(4-((7-oxabicyclo[2.2.1]heptan-2-yl)methoxy)phenyl)-2-oxo-6-(trifluoromethyl)-1,2-dihydropyridine-3-carboxamide (trans isomer, SFC peak 2) (315) was obtained after SFC purification of the product made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 2-((4-bromophenoxy)methyl)-7-oxabicyclo[2.2.1]heptane. ¹H NMR (400 MHz, DMSO-d6) δ=8.19 (s, 1H), 7.28-7.14 (m, 2H), 7.03-6.90 (m, 2H), 4.65 (t, J=4.0 Hz, 1H), 4.61-4.52 (m, 2H), 4.18-4.14 (m, 1H), 3.95 (t, J=8.0 Hz, 1H), 3.84-3.77 (m, 1H), 3.75-3.68 (m, 1H), 2.63-2.57 (m, 1H), 2.34-2.25 (m, 1H), 2.05-1.85 (m, 1H), 1.79-1.47 (m, 4H), 1.40-1.32 5 (m, 1H), 1.24-1.14 (m, 1H). 19F NMR (376 MHz, DMSO-d6) δ=−60.38 (br s, 3F). LCMS: Rt=0.73 min, m/z=409. Chiral HPLC: Rt=2.63 min, ee value=100%.

### Example 316: 5-(4-((3,8-dioxabicyclo[3.2.1]octan-2-yl)methoxy)phenyl)-2-oxo-6-(trifluoromethyl)-1,2-dihydropyridine-3-carboxamide (316)

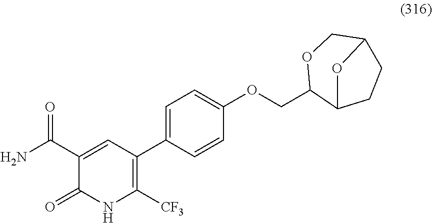 [SMILES: NC(=O)c1cc(-c2ccc(OCC3OCC4CCC3O4)cc2)c(C(F)(F)F)[nH]c1=O]

5-(4-((3,8-dioxabicyclo[3.2.1]octan-2-yl)methoxy)phenyl)-2-oxo-6-(trifluoromethyl)-1,2-dihydropyridine-3-carboxamide (316) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 2-((4-bromophenoxy)methyl)-3,8-dioxabicyclo[3.2.1]octane. ¹H NMR (400 MHz, DMSO-d6) δ=8.18 (s, 1H), 7.94 (s, 1H), 7.24 (d, J=4.4 Hz, 2H), 7.03 (d, J=8.6 Hz, 2H), 4.42-4.17 (m, 3H), 4.03-3.85 (m, 2H), 3.70 (d, J=9.4 Hz, 1H), 3.27 (s, 1H), 2.07-1.67 (m, 4H). LCMS: Rt=0.90 min, m/z=425.2 (M+H).

### 2-((4-bromophenoxy)methyl)-3,8-dioxabicyclo[3.2.1]octane was Obtained Using the Following Procedure

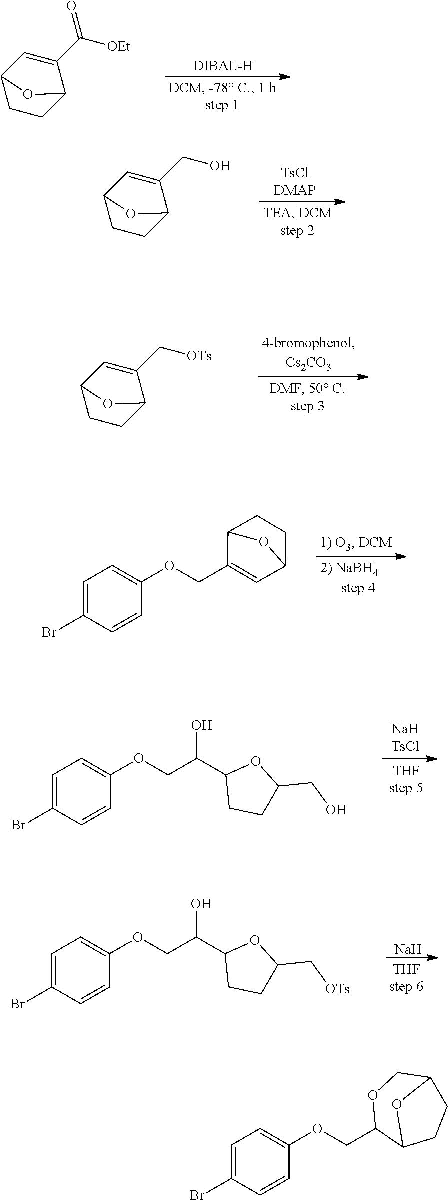

Step 1: To a solution of ethyl 7-oxabicyclo[2.2.1]hept-2-ene-2-carboxylate (7 g, 40 mmol) in DCM (100 mL) was added DIBAL-H (80 mL, 80 mmol) at −78° C. Then the mixture was stirred at −78° C. for 2 hr. The reaction mixture was poured into water (200 mL) and the resulting mixture extracted with EA (2×200 mL). The organic layers were combined, dried over anhydrous Na₂SO₄, filtered and concentrated to give crude product which was purified by prep-TLC (10:1) to give (7-oxabicyclo[2.2.1]hept-2-en-2-yl)methanol (4 g, 31 mmol). ¹H NMR (400 MHz, CDCl₃) 5.96 (d, J=1.6 Hz, 1H), 4.90 (d, J=11.4 Hz, 2H), 4.33-4.14 (m, 2H), 3.96-3.92 (m, 1H), 1.75 (t, J=7.2 Hz, 2H), 1.13 (d, J=6.0 Hz, 2H).

Step 2: (7-oxabicyclo[2.2.1]hept-2-en-2-yl)methyl 4-methylbenzenesulfonate was obtained using step 1 in the procedure to make 5-((4-bromophenoxy)methyl)-3-isopropyloxazolidin-2-one except 5-(hydroxymethyl)oxazolidin-2-one was replaced with (7-oxabicyclo[2.2.1]hept-2-en-2-yl)methanol. ¹H NMR (400 MHz, CDCl₃) 7.83-7.80 (m, 2H), 7.35 (d, J=8.0 Hz, 2H), 6.18 (d, J=1.4 Hz, 1H), 5.06-4.93 (m, 2H), 4.30-4.20 (m, 2H), 2.47 (s, 3H), 1.92-1.83 (m, 2H), 1.33 (d, J=5.4 Hz, 2H).

Step 3: 2-((4-bromophenoxy)methyl)-7-oxabicyclo[2.2.1]hept-2-ene was obtained using step 2 in the procedure to make 5-((4-bromophenoxy)methyl)-3-isopropyloxazolidin-2-one except (2-oxooxazolidin-5-yl)methyl 4-methylbenzenesulfonate was replaced with (7-oxabicyclo[2.2.1]hept-2-en-2-yl)methyl 4-methylbenzenesulfonate. ¹H NMR (400 MHz, CDCl₃) 7.41-7.37 (m, 2H), 6.86-6.77 (m, 2H), 6.18 (d, J=1.6 Hz, 1H), 5.03 (d, J=3.6 Hz, 2H), 4.68 (d, J=4.0 Hz, 2H), 1.86 (d, J=5.0 Hz, 2H), 1.33-1.29 (m, 2H).

Step 4: A solution of 2-((4-bromophenoxy)methyl)-7-oxabicyclo[2.2.1]hept-2-ene (950 mg, 3.5 mmol) in MeOH (10 mL) was stirred at −78° C. for 1 hr under ozone. Then to the mixture was added NaBH₄ (300 mg, 7.7 mmol), and the reaction was stirred at 0° C. for 1 hr. The reaction mixture was concentrated to give crude product which was purified by prep-TLC (PE:EA=1:1) to give 2-(4-bromophenoxy)-1-(5-(hydroxymethyl)tetrahydrofuran-2-yl)ethan-1-ol. ¹H NMR (400 MHz, CDCl₃) 7.43-7.37 (m, 2H), 6.83 (d, J=8.6 Hz, 2H), 4.23-3.78 (m, 6H), 3.58 (d, J=11.6 Hz, 1H), 2.11-1.98 (m, 4H).

Step 5: To a solution of 2-(4-bromophenoxy)-1-(5-(hydroxymethyl)tetrahydrofuran-2-yl)ethan-1-ol (300 mg, 1 mmol), NaH (60 mg, 1.5 mmol) in THF (3 mL) was added TsCl (190 mg, 1 mmol) at 0° C. The mixture was stirred at 25° C. for 2 hr. The reaction mixture was poured into water (10 mL) and the resulting mixture extracted with EA (2×10 mL). The organic layers were combined, dried over anhydrous Na₂SO₄, filtered and concentrated to give crude product which was purified by column chromatography (PE:EA=3:1) to give (5-(2-(4-bromophenoxy)-1-hydroxyethyl)tetrahydrofuran-2-yl)methyl 4-methylbenzenesulfonate. 1H NMR (400 MHz, CDCl₃) 7.81 (t, J=7.8 Hz, 2H), 7.44-7.31 (m, 4H), 6.89-6.75 (m, 2H), 4.24-3.92 (m, 7H), 2.45 (d, J=4.8 Hz, 3H), 2.06-1.94 (m, 3H), 1.90-1.77 (m, 1H).

Step 6: To a solution of (5-(2-(4-bromophenoxy)-1-hydroxyethyl)tetrahydrofuran-2-yl)methyl 4-methylbenzenesulfonate (100 mg, 0.2 mmol) in THF (1 mL) was added NaH (16 mg, 0.4 mmol). The mixture was stirred at 25° C. for 2 hr. The reaction mixture was poured into water (5 mL) and the resulting mixture extracted with EA (2×5 mL). The organic layers were combined, dried over anhydrous Na₂SO₄, filtered and concentrated to give crude product which was purified by Prep-TLC (PE:EA=1:1) to give 2-((4-bromophenoxy)methyl)-3,8-dioxabicyclo[3.2.1]octane. LCMS Rt=0.82 min, m/z=299.0, 301.0 (M+H).

### Example 317: 5-(4-((3,8-dioxabicyclo[3.2.1]octan-2-yl)methoxy)phenyl)-2-oxo-6-(trifluoromethyl)-1,2-dihydropyridine-3-carboxamide (SFC Peak 1) (317)

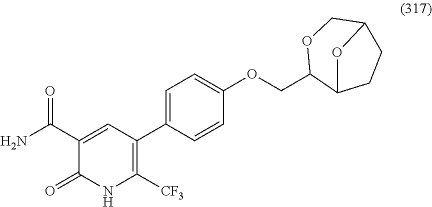 [SMILES: NC(=O)c1cc(-c2ccc(OCC3OCC4CCC3O4)cc2)c(C(F)(F)F)[nH]c1=O]

5-(4-((3,8-dioxabicyclo[3.2.1]octan-2-yl)methoxy)phenyl)-2-oxo-6-(trifluoromethyl)-1,2-dihydropyridine-3-carboxamide (SFC peak 1) (317) was isolated by SFC purification of the product from Example 316. ¹H NMR (400 MHz, DMSO-d6) δ=8.91 (s, 1H), 8.19 (s, 1H), 7.96 (s, 1H), 7.24 (d, J=8.4 Hz, 2H), 7.02 (d, J=8.6 Hz, 2H), 4.22 (t, J=6.2 Hz, 2H), 4.03-3.84 (m, 3H), 3.70 5 (d, J=10.8 Hz, 1H), 3.51 (d, J=11.2 Hz, 1H), 2.08-1.98 (m, 1H), 1.92-1.80 (m, 2H), 1.79-1.67 (m, 1H). LCMS: Rt=0.69 min, m/z=425.5 (M+H). Chiral HPLC: Rt=2.51 min, de value=13.7%.

### Example 318: 5-(4-((3,8-dioxabicyclo[3.2.1]octan-2-yl)methoxy)phenyl)-2-oxo-6-(trifluoromethyl)-1,2-dihydropyridine-3-carboxamide (SFC Peak 2) (318)

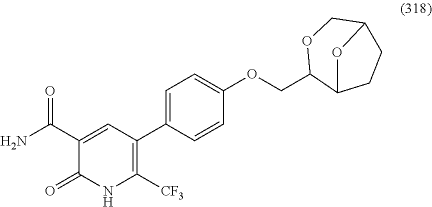 [SMILES: NC(=O)c1cc(-c2ccc(OCC3OCC4CCC3O4)cc2)c(C(F)(F)F)[nH]c1=O]

5-(4-((3,8-dioxabicyclo[3.2.1]octan-2-yl)methoxy)phenyl)-2-oxo-6-(trifluoromethyl)-1,2-dihydropyridine-3-carboxamide (SFC peak 2) (318) was isolated by SFC purification of the product from Example 316. ¹H NMR (400 MHz, DMSO-d6) δ=8.08 (d, J=10.4 Hz, 1H), 7.22 (d, J=8.4 Hz, 2H), 7.10-6.93 (m, 2H), 4.43-4.15 (m, 4H), 3.90 (d, J=11.8 Hz, 1H), 3.69 (t, J=6.4 Hz, 1H), 3.27 (s, 1H), 2.05-1.79 (m, 4H). LCMS: Rt=0.691 min, m/z=425.5 (M+H). Chiral HPLC: Rt=2.56 min, de value=65.9%.

### Example 319: 5-(4-((3,8-dioxabicyclo[3.2.1]octan-2-yl)methoxy)phenyl)-2-oxo-6-(trifluoromethyl)-1,2-dihydropyridine-3-carboxamide (SFC Peak 3) (319)

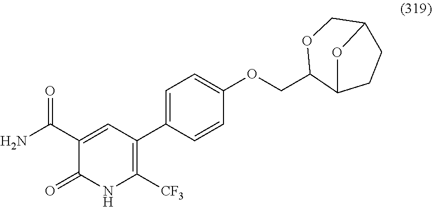 [SMILES: NC(=O)c1cc(-c2ccc(OCC3OCC4CCC3O4)cc2)c(C(F)(F)F)[nH]c1=O]

5-(4-((3,8-dioxabicyclo[3.2.1]octan-2-yl)methoxy)phenyl)-2-oxo-6-(trifluoromethyl)-1,2-dihydropyridine-3-carboxamide (SFC peak 3 was isolated by SFC purification of the product from Example 316. ¹H NMR (400 MHz, DMSO-d6) δ=8.12 (s, 1H), 7.28-7.13 (m, 2H), 7.07-6.94 (m, 2H), 4.41-4.13 (m, 4H), 3.90 (d, J=10.8 Hz, 1H), 3.69 (t, J=6.4 Hz, 1H), 3.27 (s, 1H), 2.05-1.73 (m, 4H). LCMS: Rt=0.69 min, m/z=425.5 (M+H). Chiral HPLC: Rt=3.07 min, de value=82.2%.

### Example 320: 5-(4-((1,4-dioxepan-6-yl)methoxy)phenyl)-2-oxo-6-(trifluoromethyl)-1,2-dihydropyridine-3-carboxamide (320)

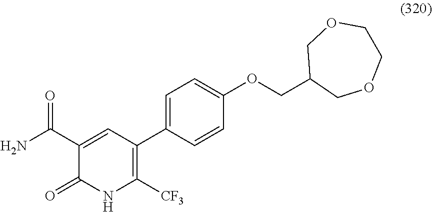 [SMILES: NC(=O)c1cc(-c2ccc(OCC3COCCOC3)cc2)c(C(F)(F)F)[nH]c1=O]

5-(4-((1,4-dioxepan-6-yl)methoxy)phenyl)-2-oxo-6-(trifluoromethyl)-1,2-dihydropyridine-3-carboxamide (320) was made using a procedure similar to steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 6-((4-bromophenoxy)methyl)-1,4-dioxepane. ¹H NMR (400 MHz, CDCl₃) δ=10.89 (d, J=5.6 Hz, 1H), 7.77 (s, 1H), 7.11 (d, J=8.4 Hz, 2H), 6.98-6.83 (m, 3H), 3.99-3.85 (m, 4H), 3.74-3.62 (m, 6H), 2.47-2.43 (m, 1H). LCMS: Rt=0.68 min, m/z=413.2 (M+H).

### Example 321: 5-(4-((6-methyl-1,4-dioxepan-6-yl)methoxy)phenyl)-2-oxo-6-(trifluoromethyl)-1,2-dihydropyridine-3-carboxamide (321)

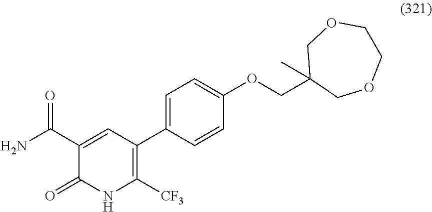 [SMILES: CC1(COc2ccc(-c3cc(C(N)=O)c(=O)[nH]c3C(F)(F)F)cc2)COCCOC1]

5-(4-((6-methyl-1,4-dioxepan-6-yl)methoxy)phenyl)-2-oxo-6-(trifluoromethyl)-1,2-dihydropyridine-3-carboxamide (321) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (6-methyl-1,4-dioxepan-6-yl)methanol. ¹H NMR: (400 MHz, CDCl₃) δ=8.84 (s, 1H), 8.26-8.13 (m, 1H), 7.98 (s, 1H), 7.24 (d, J=8.6 Hz, 2H), 7.01 (d, J=8.6 Hz, 2H), 3.82 (s, 2H), 3.75-3.67 (m, 5H), 3.50 (d, J=12.4 Hz, 1H), 2.08 (s, 2H), 0.96 (s, 3H). LCMS: Rt=0.72 min, m/z=427.2 (M+H).

### (6-methyl-1,4-dioxepan-6-yl)methanol was Obtained Using the Following Procedure

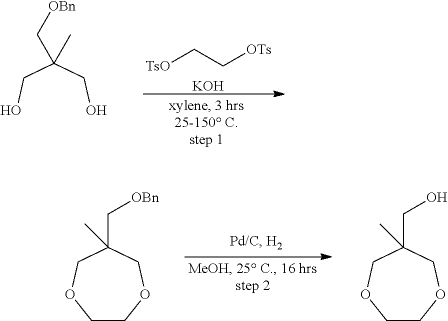

Step 1: To a solution of 2-((benzyloxy)methyl)-2-methylpropane-1,3-diol (see J. Org. Chem., 1987, 52, 2420-2427) (2.3 g, 11 mmol), ethane-1,2-diyl bis(4-methylbenzenesulfonate) (see J. Org. Chem., 1982, 47, 412-415) (4.1 g, 11 mmol) in xylene (50 mL) was added KOH (22 g, 44 mmol) at 25° C. and the mixture stirred at 150° C. for 3 hrs. The reaction mixture was poured into water (100 mL) and then extracted with EA (100 mL×2). The organic layers were combined, dried over anhydrous Na₂SO₄, filtered and concentrated to give crude product which was purified by column chromatography (PE:EA=10:1˜5:1) to give 6-((benzyloxy)methyl)-6-methyl-1,4-dioxepane. LCMS: Rt=0.78 min, m/z=237.2 (M+H).

Step 2: To a solution of 6-((benzyloxy)methyl)-6-methyl-1,4-dioxepane (150 mg, 1 mmol) in MeOH (5 mL) was added Pd/C (20 mg) at 25° C. Then the mixture was stirred at 25° C. for 16 hr under H₂ (15 psi). TLC (PE/EA=1:1) showed the starting material was consumed and one new major spot (Rf=0.1) was detected. The reaction mixture was filtered and the filtrate was concentrated to give the crude product. The crude product was purified by column chromatography (PE:EA=5:1˜1:1) to give (6-methyl-1,4-dioxepan-6-yl)methanol. ¹H NMR (400 MHz, CDCl₃) δ=3.89-3.77 (m, 6H), 3.59 (d, J=12.5 Hz, 2H), 3.52 (s, 2H), 0.87 (s, 3H).

### Example 322: 5-(4-((5-methyl-1,4-dioxan-2-yl)methoxy)phenyl)-2-oxo-6-(trifluoromethyl)-1,2-dihydropyridine-3-carboxamide (Cis Isomer, SFC Peak 1) (322)

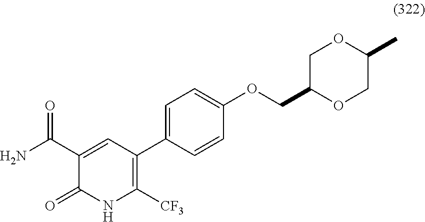

5-(4-((5-methyl-1,4-dioxan-2-yl)methoxy)phenyl)-2-oxo-6-(trifluoromethyl)-1,2-dihydropyridine-3-carboxamide (cis isomer, SFC peak 1) (322) was obtained after SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-methyl-1,4-dioxan-2-yl)methanol (see Bioorg. Med. Chem., 2009, 17, 8174-8185). ¹H NMR: (400 MHz, DMSO-d6) δ=8.69 (s, 1H), 8.24 (s, 1H), 8.06 (s, 1H), 7.27 (d, J=8.6 Hz, 2H), 7.06 (d, J=8.6 Hz, 2H), 4.37-4.11 (m, 2H), 3.91 (d, J=3.4 Hz, 1H), 3.83-3.76 (m, 1H), 3.74-3.65 (m, 2H), 3.62-3.55 (m, 1H), 3.52-3.46 (m, 1H), 1.10 (d, J=6.4 Hz, 3H). LCMS: Rt=0.69 min, m/z=413.1 (M+H). Chiral HPLC: Rt=1.87 min, ee value=75.4%.

### Example 323: 5-(4-((5-methyl-1,4-dioxan-2-yl)methoxy)phenyl)-2-oxo-6-(trifluoromethyl)-1,2-dihydropyridine-3-carboxamide (Cis Isomer, SFC Peak 2) (323)

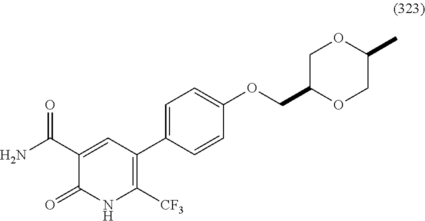

5-(4-((5-methyl-1,4-dioxan-2-yl)methoxy)phenyl)-2-oxo-6-(trifluoromethyl)-1,2-dihydropyridine-3-carboxamide (cis isomer, SFC peak 2) (323) was obtained by SFC of the product from Example 322. ¹H NMR: (400 MHz, DMSO-d6) δ=8.60 (s, 1H), 8.26 (s, 1H), 8.11 (s, 1H), 7.28 (d, J=8.6 Hz, 2H), 7.17-7.01 (m, 2H), 4.29 (d, J=10.2 Hz, 1H), 4.22-4.15 (m, 1H), 3.95-3.87 (m, 1H), 3.83-3.77 (m, 1H), 3.74-3.65 (m, 2H), 3.62-3.55 (m, 1H), 3.53-3.46 (m, 1H), 1.10 (d, J=6.4 Hz, 3H). LCMS: Rt=0.79 min, m/z=413.1 (M+H). Chiral HPLC: R=1.97 min, ee value=95.2%.

### Example 324: 5-(4-((5-methyl-1,4-dioxan-2-yl)methoxy)phenyl)-2-oxo-6-(trifluoromethyl)-1,2-dihydropyridine-3-carboxamide (Trans Isomer, Racemate) (324)

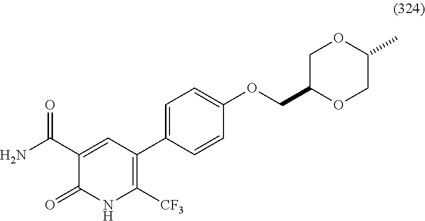

5-(4-((5-methyl-1,4-dioxan-2-yl)methoxy)phenyl)-2-oxo-6-(trifluoromethyl)-1,2-dihydropyridine-3-carboxamide (trans isomer, racemate) (324) was obtained by SFC of the product from Example 322. ¹H NMR: (400 MHz, DMSO-d6) δ=9.85 (s, 1H), 8.21 (s, 1H), 7.97 (s, 1H), 7.46 (s, 1H), 7.17 (d, J=8.4 Hz, 2H), 7.04-6.87 (m, 2H), 3.97 (d, J=4.8 Hz, 2H), 3.91-3.84 (m, 1H), 3.82-3.74 (m, 2H), 3.59-3.54 (m, 1H), 3.48-3.42 (m, 1H), 3.26-3.19 (m, 1H), 1.01 (d, J=6.2 Hz, 3H). LCMS: Rt=0.71 min, m/z=413.1 (M+H).

### Example 325: 5-(4-(((2R,6R)-6-cyclopropyl-1,4-dioxan-2-yl)methoxy)phenyl)-2-oxo-6-(trifluoromethyl)-1,2-dihydropyridine-3-carboxamide (325)

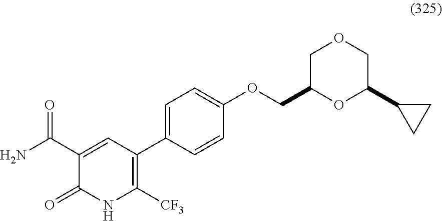

5-(4-(((2R,6R)-6-cyclopropyl-1,4-dioxan-2-yl)methoxy)phenyl)-2-oxo-6-(trifluoromethyl)-1,2-dihydropyridine-3-carboxamide (325) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with cis-(6-cyclopropyl-1,4-dioxan-2-yl)methanol. ¹H NMR (400 MHz, CDCl₃) δ=9.13 (s, 1H), 8.57 (s, 1H), 7.21 (d, J=8.0 Hz, 2H), 6.95 (d, J=8.0 Hz, 2H), 6.14 (s, 1H), 4.10 (m, 1H), 4.05-3.92 (m, 3H), 3.87 (m, 1H), 3.52-3.35 (m, 2H), 3.04-2.91 (m, 1H), 0.86-0.73 (m, 1H), 0.65-0.49 (m, 2H), 0.44 (m, 1H), 0.26 (m, 1H). LCMS: Rt=0.78 min, m/z=439.3 (M+H). Chiral HPLC: Rt=2.46 min, ee value=92.4%.

### cis-(6-cyclopropyl-1,4-dioxan-2-yl)methanol and trans-(6-cyclopropyl-1,4-dioxan-2-yl)methanol were Obtained Using the Following Procedure

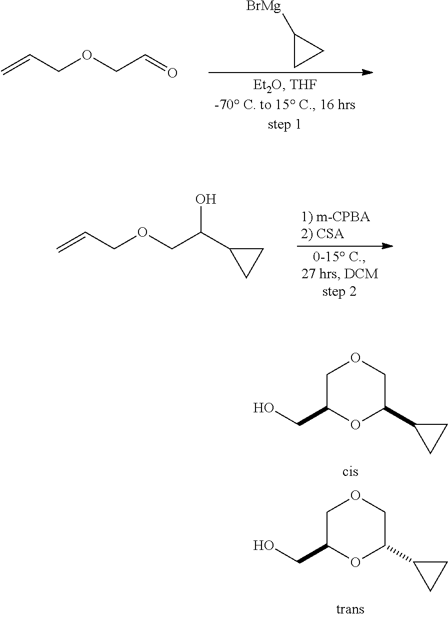

Step 1: Cyclopropylmagnesium bromide (500 mL, 254.71 mmol, 0.5M in THF) was added to 2-(allyloxy)acetaldehyde (150 ml, 169.80 mmol, 1M in THF) in THF (150 mL) dropwise at −70° C. and the mixture stirred at 15° C. for 16 hrs. The reaction mixture was quenched with sat. aq. NH₄Cl (300 mL), then extracted with EA (2×100 mL). The organic layers were combined, dried over anhydrous Na₂SO₄, filtered and concentrated to give crude product which was purified by silica gel (PE) to give 2-(allyloxy)-1-cyclopropylethan-1-ol. ¹H NMR (400 MHz, CDCl₃) δ=5.79-5.64 (m, 1H), 5.13-4.94 (m, 2H), 3.86-3.78 (m, 2H), 3.61-3.50 (m, 1H), 3.38 (m, 1H), 3.22 (m, 1H), 2.92-2.83 (m, 1H), 2.14 (m, 1H), 1.16-0.97 (m, 1H), 0.38-0.23 (m, 2H), 0.17 (m, 1H), 0.01 (m, 1H).

Step 2: To a solution of 2-(allyloxy)-1-cyclopropylethan-1-ol (4.6 g, 32.35 mmol) in DCM (50 mL) was added m-CPBA (7.26 g, 42.05 mmol) in one portion at 0° C. The mixture was stirred at 15° C. for 24 hrs. The formed solid was filtered out. To the resulting solution was added CSA (2.26 g, 9.71 mmol) and the mixture stirred for a further 3 hrs. The reaction mixture was quenched with sat. aq. Na₂CO₃ solution (100 mL), then extracted with DCM (2×50 mL). The organic layers were combined, dried over anhydrous Na₂SO₄, filtered and concentrated to give crude product. The crude product was purified by silica gel column chromatograph (PE to PE/EA=16/1) to give cis-(6-cyclopropyl-1,4-dioxan-2-yl)methanol and trans-(6-cyclopropyl-1,4-dioxan-2-yl)methanol.

Cis-(6-cyclopropyl-1,4-dioxan-2-yl)methanol isomer ¹H NMR (400 MHz, MeOD-d4) δ=3.79 (m, 2H), 3.65-3.46 (m, 3H), 3.38-3.25 (m, 5H), 2.92 (m, 1H), 0.81-0.71 (m, 1H), 0.55-0.45 (m, 2H), 0.44-0.36 (m, 1H), 0.32-0.23 (m, 1H).

### Example 326: 5-(4-(((2S,8S)-6-cyclopropyl-1,4-dioxan-2-yl)methoxy)phenyl)-2-oxo-6-(trifluoromethyl)-1,2-dihydropyridine-3-carboxamide (326)

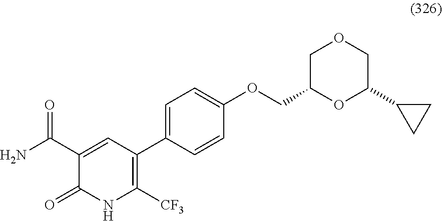 [SMILES: NC(=O)c1cc(-c2ccc(OC[C@@H]3COC[C@H](C4CC4)O3)cc2)c(C(F)(F)F)[nH]c1=O]

5-(4-(((2S,6S)-6-cyclopropyl-1,4-dioxan-2-yl)methoxy)phenyl)-2-oxo-6-(trifluoromethyl)-1,2-dihydropyridine-3-carboxamide (326) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with cis-(6-cyclopropyl-1,4-dioxan-2-yl)methanol. The racemic mixture was separated by chiral prep-HPLC. ¹H NMR (500 MHz, DMSO-d6) δ 13.65 (s, 1H), 8.48 (s, 1H), 8.29 (s, 1H), 8.19 (s, 1H), 7.38-7.19 (m, 2H), 7.11-6.97 (m, 2H), 4.05 (dd, J=10.5, 5.4 Hz, 1H), 4.00 (dd, J=10.4, 4.4 Hz, 1H), 3.89 (ddd, J=10.4, 4.9, 2.8 Hz, 1H), 3.87-3.83 (m, 1H), 3.78 (dd, J=11.4, 2.6 Hz, 1H), 3.32 (d, J=9.8 Hz, 1H), 3.27 (d, J=11.0 Hz, 1H), 2.97 (ddd, J=10.5, 8.2, 2.6 Hz, 1H), 0.76 (qt, J=8.3, 4.9 Hz, 1H), 0.50-0.40 (m, 2H), 0.33 (dtd, J=9.0, 4.6, 2.8 Hz, 1H), 0.30-0.24 (m, 1H). LCMS Rt=0.75 min, m/z=439.3 (M+H). Chiral HPLC: Rt=2.45 min, ee value=99%.

### cis-(6-cyclopropyl-1,4-dioxan-2-yl)methanol and trans-(6-cyclopropyl-1,4-dioxan-2-yl)methanol and trans-(6-cyclopropyl-1,4-dioxan-2-yl)methanol and trans-(6-cyclopropyl-1,4-dioxan-2-yl)methanol were Obtained Using the Following Procedure

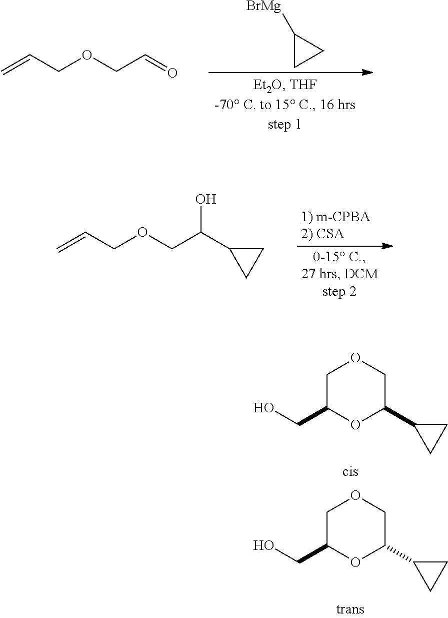

Step 1: Cyclopropylmagnesium bromide (500 mL, 254.71 mmol, 0.5M in THF) was added to 2-(allyloxy)acetaldehyde (150 ml, 169.80 mmol, 1M in THF) in THF (150 mL) dropwise at −70° C. and the mixture stirred at 15° C. for 16 hrs. The reaction mixture was quenched with sat. aq. NH₄Cl (300 mL), then extracted with EA (2×100 mL). The organic layers were combined, dried over anhydrous Na₂SO₄, filtered and concentrated to give crude product which was purified by silica gel (PE) to give 2-(allyloxy)-1-cyclopropylethan-1-ol. ¹H NMR (400 MHz, CDCl₃) δ=5.79-5.64 (m, 1H), 5.13-4.94 (m, 2H), 3.86-3.78 (m, 2H), 3.61-3.50 (m, 1H), 3.38 (m, 1H), 3.22 (m, 1H), 2.92-2.83 (m, 1H), 2.14 (m, 1H), 1.16-0.97 (m, 1H), 0.38-0.23 (m, 2H), 0.17 (m, 1H), 0.01 (m, 1H).

Step 2: To a solution of 2-(allyloxy)-1-cyclopropylethan-1-ol (4.6 g, 32.35 mmol) in DCM (50 mL) was added m-CPBA (7.26 g, 42.05 mmol) in one portion at 0° C. The mixture was stirred at 15° C. for 24 hrs. The formed solid was filtered out. To the resulting solution was added CSA (2.26 g, 9.71 mmol) and the mixture stirred for a further 3 hrs. The reaction mixture was quenched with sat. aq. Na₂CO₃ solution (100 mL), then extracted with DCM (2×50 mL). The organic layers were combined, dried over anhydrous Na₂SO₄, filtered and concentrated to give crude product. The crude product was purified by silica gel column chromatograph (PE to PE/EA=16/1) to give cis-(6-cyclopropyl-1,4-dioxan-2-yl)methanol (880 mg) and trans-(6-cyclopropyl-1,4-dioxan-2-yl)methanol (950 mg).

Cis-(6-cyclopropyl-1,4-dioxan-2-yl)methanol isomer ¹H NMR (400 MHz, MeOD-d4) δ=3.79 (m, 2H), 3.65-3.46 (m, 3H), 3.38-3.25 (m, 2H), 2.92 (m, 1H), 0.81-0.71 (m, 1H), 0.55-0.45 (m, 2H), 0.44-0.36 (m, 1H), 0.32-0.23 (m, 1H).

Trans-(6-cyclopropyl-1,4-dioxan-2-yl)methanol isomer ¹H NMR (400 MHz, MeOD-d4) δ=3.85 (m, 1H), 3.75-3.71 (m, 2H), 3.69-3.60 (m, 2H), 3.59-3.51 (m, 2H), 2.94 (m, 1H), 1.11 (m, 1H), 0.55-0.49 (m, 2H), 0.37 (m, 1H), 0.24 (m, 1H).

### Example 327: 5-(4-(((2R,6S)-6-cyclopropyl-1,4-dioxan-2-yl)methoxy)phenyl)-2-oxo-6-(trifluoromethyl)-1,2-dihydropyridine-3-carboxamide (326)

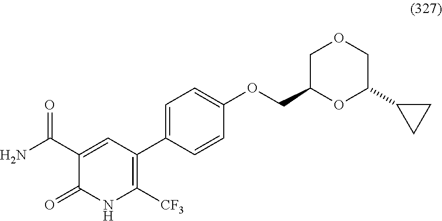 [SMILES: NC(=O)c1cc(-c2ccc(OC[C@H]3COC[C@H](C4CC4)O3)cc2)c(C(F)(F)F)[nH]c1=O]

5-(4-(((2R,6S)-6-cyclopropyl-1,4-dioxan-2-yl)methoxy)phenyl)-2-oxo-6-(trifluoromethyl)-1,2-dihydropyridine-3-carboxamide (327) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with trans-(6-cyclopropyl-1,4-dioxan-2-yl)methanol. ¹H NMR (400 MHz, CDCl₃) δ=9.13 (s, 1H), 8.57 (s, 1H), 7.22 (d, J=8.0 Hz, 2H), 6.98 (d, J=8.0 Hz, 2H), 6.10 (s, 1H), 4.33-4.11 (m, 3H), 3.91-3.82 (m, 2H), 3.81-3.75 (m, 1H), 3.63-3.58 (m, 1H), 3.06-2.96 (m, 1H), 1.16-1.05 (m, 1H), 0.66-0.52 (m, 2H), 0.43-0.37 (m, 1H), 0.28-0.19 (m, 1H). LCMS: Rt=0.74 min, m/z=439 (M+H). Chiral HPLC: Rt=2.52 min, ee value=100%.

### Example 328: 5-(4-(((2R,6R)-6-methyl-1,4-dioxan-2-yl)methoxy)phenyl)-2-oxo-6-(trifluoromethyl)-1,2-dihydropyridine-3-carboxamide (328)

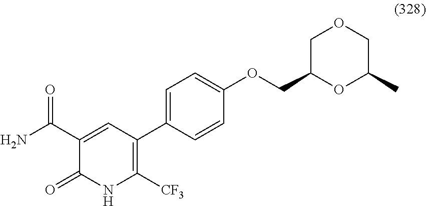 [SMILES: C[C@@H]1COC[C@H](COc2ccc(-c3cc(C(N)=O)c(=O)[nH]c3C(F)(F)F)cc2)O1]

5-(4-(((2R,6R)-6-methyl-1,4-dioxan-2-yl)methoxy)phenyl)-2-oxo-6-(trifluoromethyl)-1,2-dihydropyridine-3-carboxamide (328) was made by the same procedure used to make 5-(4-(((2R,6R)-6-cyclopropyl-1,4-dioxan-2-yl)methoxy)phenyl)-2-oxo-6-(trifluoromethyl)-1,2-dihydropyridine-3-carboxamide (327) except cis-(6-cyclopropyl-1,4-dioxan-2-yl)methanol was replaced with ((2S,6R)-6-methyl-1,4-dioxan-2-yl)methanol. ¹H NMR (400 MHz, DMSO-d6) δ=8.53 (br s, 1H), 8.27 (s, 1H), 8.15 (br s, 1H), 7.26 (br d, J=8.4 Hz, 2H), 7.03 (d, J=8.7 Hz, 2H), 4.10-3.79 (m, 4H), 3.77-3.61 (m, 2H), 3.42-3.23 (m, 2H), 3.08 (t, J=11.0 Hz, 1H), 2.54-2.47 (m, 5H), 1.03 (d, J=6.1 Hz, 3H). LCMS: Rt=0.73 min, m/z=413.1 (M+H). Chiral HPLC: Rt=2.11 min, de value=99.1%.

((2S,6R)-6-methyl-1,4-dioxan-2-yl)methanol was obtained using the procedure used to obtain cis-(6-cyclopropyl-1,4-dioxan-2-yl)methanol, except cyclopropylmagnesium bromide was replaced with methylmagnesium bromide.

### Example 329: 5-(4-(((2S,6R)-6-methyl-1,4-dioxan-2-yl)methoxy)phenyl)-2-oxo-6-(trifluoromethyl)-1,2-dihydropyridine-3-carboxamide (329)

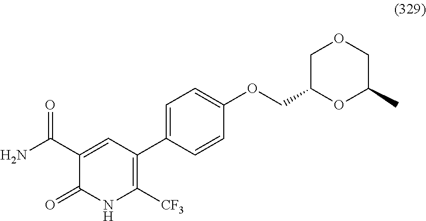 [SMILES: C[C@@H]1COC[C@@H](COc2ccc(-c3cc(C(N)=O)c(=O)[nH]c3C(F)(F)F)cc2)O1]

5-(4-(((2S,6R)-6-methyl-1,4-dioxan-2-yl)methoxy)phenyl)-2-oxo-6-(trifluoromethyl)-1,2-dihydropyridine-3-carboxamide (329) was made by the same procedure used to make 5-(4-(((2R,6S)-6-cyclopropyl-1,4-dioxan-2-yl)methoxy)phenyl)-2-oxo-6-(trifluoromethyl)-1,2-dihydropyridine-3-carboxamide (327) except trans-(6-cyclopropyl-1,4-dioxan-2-yl))methanol was replaced with ((2S,6S)-6-methyl-1,4-dioxan-2-yl)methanol. ¹H NMR (400 MHz, DMSO-d6) δ=9.13 (br s, 1H), 8.14 (s, 1H), 7.82 (br s, 1H), 7.24 (d, J=8.8 Hz, 2H), 7.04 (d, J=8.8 Hz, 2H), 4.23-3.23 (m, 8H), 1.08 (d, 3H). LCMS: Rt=0.71 min, m/z=413.3 (M+H). Chiral HPLC: Rt=2.54 min, de value=98.7%.

((2S,6R)-6-methyl-1,4-dioxan-2-yl)methanol was obtained using the procedure used to obtain trans-(6-cyclopropyl-1,4-dioxan-2-yl)methanol, except cyclopropylmagnesium bromide was replaced with methylmagnesium bromide.

### Example 330: 5-(4-(((2S,6S)-6-methyl-1,4-dioxan-2-yl)methoxy)phenyl)-2-oxo-6-(trifluoromethyl)-1,2-dihydropyridine-3-carboxamide (330)

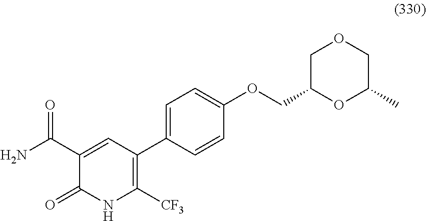 [SMILES: C[C@H]1COC[C@@H](COc2ccc(-c3cc(C(N)=O)c(=O)[nH]c3C(F)(F)F)cc2)O1]

5-(4-(((2S,6S)-6-methyl-1,4-dioxan-2-yl)methoxy)phenyl)-2-oxo-6-(trifluoromethyl)-1,2-dihydropyridine-3-carboxamide (329) was obtained by prep-HPLC from the mixture containing Example 327. ¹H NMR (400 MHz, DMSO-d6) δ=8.49 (br s, 1H), 8.27 (s, 1H), 8.15 (br s, 1H), 7.28 (d, J=8.6 Hz, 2H), 7.06 (d, J=8.6 Hz, 2H), 4.01 (m, 3H), 3.99 (m, 1H), 3.71 (m, 2H), 3.32 (s, 1H), 3.08 (m, 1H) 1.04 (d, J=6.4 Hz, 3H). LCMS: Rt=0.69 min, m/z=413.4 (M+H). Chiral HPLC: Rt=1.64 min, de value=96.1%.

### Example 331: 5-(4-(((2R,6S)-6-methyl-1,4-dioxan-2-yl)methoxy)phenyl)-2-oxo-6-(trifluoromethyl)-1,2-dihydropyridine-3-carboxamide (331)

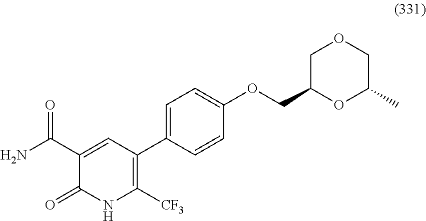 [SMILES: C[C@H]1COC[C@H](COc2ccc(-c3cc(C(N)=O)c(=O)[nH]c3C(F)(F)F)cc2)O1]

5-(4-(((2R,6S)-6-methyl-1,4-dioxan-2-yl)methoxy)phenyl)-2-oxo-6-(trifluoromethyl)-1,2-dihydropyridine-3-carboxamide (331) was obtained by prep-HPLC from the mixture containing Example 328. ¹H NMR (400 MHz, DMSO-d6) δ=8.83 (br s, 1H), 8.20 (s, 1H), 7.99 (br s, 1H), 7.26 (d, J=8.6 Hz, 2H), 7.06 (d, J=8.6 Hz, 2H), 4.24 (m, 2H), 4.20 (m, 1H), 3.70 (m, 2H), 3.33 (m, 1H), 1.09 (d, J=6.4 Hz, 3H). LCMS: Rt=0.69 min, m/z=413.3 (M+H). Chiral HPLC: Rt=2.19 min, de value=98.4%.

### Example 332: 5-(4-((6-cyclopentyl-1,4-dioxan-2-yl)methoxy)phenyl)-2-oxo-6-(trifluoromethyl)-1,2-dihydropyridine-3-carboxamide (cis racemate) (332)

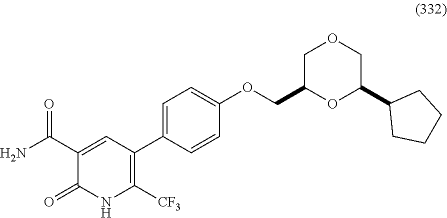

5-(4-((6-cyclopentyl-1,4-dioxan-2-yl)methoxy)phenyl)-2-oxo-6-(trifluoromethyl)-1,2-dihydropyridine-3-carboxamide (cis racemate) (332) was made by the same procedure used to make 5-(4-(((2R,6R)-6-cyclopropyl-1,4-dioxan-2-yl)methoxy)phenyl)-2-oxo-6-(trifluoromethyl)-1,2-dihydropyridine-3-carboxamide (325) except cis-(6-cyclopropyl-1,4-dioxan-2-yl)methanol was replaced with ((2S,6R)-6-cyclopentyl-1,4-dioxan-2-yl)methanol. ¹H NMR (400 MHz, CDCl₃) δ=12.40 (s, 1H), 9.15 (d, J=4.0 Hz, 1H), 8.58 (s, 1H), 7.22 (d, J=8.0 Hz, 2H), 6.97 (d, J=8.0 Hz, 2H), 6.21 (d, J=4.0 Hz, 1H), 4.11-4.06 (m, 1H), 4.06-3.99 (m, 1H), 3.99-3.90 (m, 2H), 3.81 (m, 1H), 3.50-3.38 (m, 2H), 3.34-3.22 (m, 1H), 1.88-1.77 (m, 2H), 1.70-1.40 (m, 6H), 1.28-1.16 (m, 1H); LCMS: Rt=0.89 min, m/z=467.0 (M+H).

((2S,6R)-6-cyclopentyl-1,4-dioxan-2-yl)methanol was obtained using the procedure used to obtain trans-(6-cyclopropyl-1,4-dioxan-2-yl)methanol, except cyclopropylmagnesium bromide was replaced with cyclopentylmagnesium bromide.

### Example 333: 5-(4-((6-cyclopentyl-1,4-dioxan-2-yl)methoxy)phenyl)-2-oxo-6-(trifluoromethyl)-1,2-dihydropyridine-3-carboxamide (trans racemate) (333)

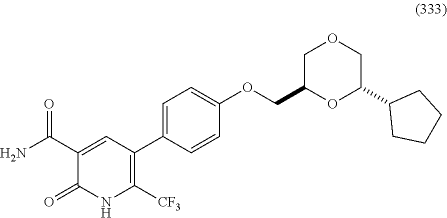

5-(4-((6-cyclopentyl-1,4-dioxan-2-yl)methoxy)phenyl)-2-oxo-6-(trifluoromethyl)-1,2-dihydropyridine-3-carboxamide (trans racemate) (333) was made by the same procedure used to make 5-(4-(((2R,6S)-6-cyclopropyl-1,4-dioxan-2-yl)methoxy)phenyl)-2-oxo-6-(trifluoromethyl)-1,2-dihydropyridine-3-carboxamide (327) except trans-(6-cyclopropyl-1,4-dioxan-2-yl)methanol was replaced with ((2S,6S)-6-cyclopentyl-1,4-dioxan-2-yl)methanol. ¹H NMR (400 MHz, CDCl₃) δ=11.89 (s, 1H), 9.13 (s, 1H), 8.58 (s, 1H), 7.22 (d, J=8.0 Hz, 2H), 6.99 (d, J=8.0 Hz, 2H), 6.06 (s, 1H), 4.25-4.17 (m, 2H), 4.16 (d, J=4.0 Hz, 1H), 3.90-3.69 (m, 3H), 3.57-3.43 (m, 2H), 1.97-1.75 (m, 1H), 1.74-1.53 (m, 6H), 1.52-1.36 (m, 1H), 1.23-1.05 (m, 1H). LCMS: Rt=0.87 min, m/z=467.0 (M+H).

((2S,6S)-6-cyclopentyl-1,4-dioxan-2-yl)methanol was obtained using the procedure used to obtain trans-(6-cyclopropyl-1,4-dioxan-2-yl)methanol, except cyclopropylmagnesium bromide was replaced with cyclopentylmagnesium bromide.

### Example 334: 5-(4-((4,7-dioxaspiro[2.5]octan-5-yl)methoxy)phenyl)-2-oxo-6-(trifluoromethyl)-1,2-dihydropyridine-3-carboxamide (SFC Peak 1) (334)

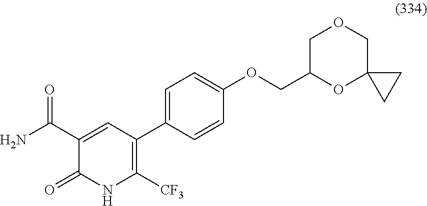 [SMILES: NC(=O)c1cc(-c2ccc(OCC3COCC4(CC4)O3)cc2)c(C(F)(F)F)[nH]c1=O]

5-(4-((4,7-dioxaspiro[2.5]octan-5-yl)methoxy)phenyl)-2-oxo-6-(trifluoromethyl)-1,2-dihydropyridine-3-carboxamide (SFC peak 1) (334) was obtained by SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4,7-dioxaspiro[2.5]octan-5-ylmethanol. ¹H NMR (400 MHz, MeOD-d4) δ=8.17 (s, 1H), 7.20 (d, J=8.5 Hz, 2H), 6.95 (d, J=8.8 Hz, 2H), 4.18-4.11 (m, 1H), 4.07-3.94 (m, 4H), 3.63 (dd, J=9.9, 11.5 Hz, 1H), 3.25 (d, J=11.5 Hz, 1H), 0.93-0.77 (m, 2H), 0.69-0.58 (m, 2H). 19F NMR (376 MHz, MeOD-d4) δ=−62.91 (s, 3F). LCMS: Rt=0.76 min, m/z=424.9 (M+H). Chiral HPLC: Rt=1.77 min, ee value 86%.

### (4,7-dioxaspiro[2.5]octan-5-yl)methanol was Obtained Using the Following Procedure

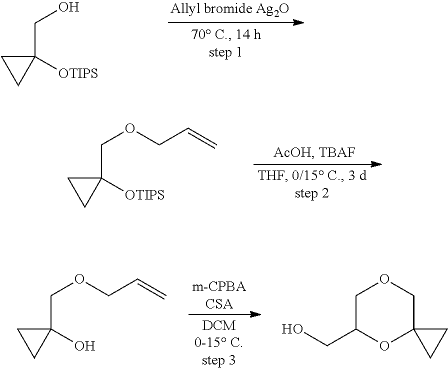

Step 1: To a solution of (1-((triisopropylsilyl)oxy)cyclopropyl)methanol (see Bioorg. Med. Chem. Left., 2001, 11, 619-622) (2.8 g, 11.45 mmol) and Ag₂O (10.6 g, 45.8 mmol) was added allyl bromide (17 mL) under N₂ in the dark at 15° C. The mixture was stirred at 70° C. for 14 h. The reaction mixture was filtered and the filtrate concentrated to give a residue which was purified by silica gel chromatography (PE/EA=200/1) to give (1-((allyloxy)methyl)cyclopropoxy)triisopropylsilane. ¹H NMR (400 MHz, DMSO-d6) δ=5.94-5.82 (m, 1H), 5.24 (qd, J=1.8, 17.3 Hz, 1H), 5.16-5.10 (m, 1H), 3.96 (td, J=1.4, 5.3 Hz, 2H), 3.45 (s, 2H), 1.04-1.00 (m, 21H), 0.71-0.59 (m, 4H).

Step 2: To a solution of (1-((allyloxy)methyl)cyclopropoxy)triisopropylsilane (3.7 g, 13 mmol) in THF (25 mL) was added TBAF (6.8 g, 26 mmol) and AcOH (1.56 g, 26 mmol) at 0° C. under N₂. The mixture was stirred at 15° C. for 3 d. The reaction mixture was concentrated to give crude product which was purified by silica gel chromotography (PE/EA=30/1) to give 1-((allyloxy)methyl)cyclopropan-1-ol. ¹H NMR: (400 MHz, DMSO-d6) δ=5.88 (tdd, J=5.3, 10.5, 17.3 Hz, 1H), 5.25 (qd, J=1.8, 17.3 Hz, 1H), 5.12 (qd, J=1.6, 10.5 Hz, 1H), 4.00-3.95 (m, 2H), 3.38 (s, 2H), 0.59-0.52 (m, 2H), 0.48-0.41 (m, 2H).

Step 3: To a solution of 1-((allyloxy)methyl)cyclopropan-1-ol (600 mg, 4.68 mmol) in DCM (6 mL) was added m-CPBA (1.2 g, 6.09 mmol) in one portion at 0° C. The mixture was stirred at 15° C. for 2 hrs. To the resulting mixture was added CSA (326 mg, 1.40 mmol) and the mixture stirred for a further 3 hrs. The reaction mixture was washed with sat. aq. Na₂CO₃ (200 mL) and extracted with DCM (20 mL×10) and DCM/i-PrOH (V/V=3/1, 20 mL×3). The organic layers were combined, dried over Na₂SO₄, filtered and concentrated to give crude product which was purified by silica gel column chromatograph (PE/EA=200/1 to 10/1) to give (4,7-dioxaspiro[2.5]octan-5-yl)methanol. ¹H NMR (400 MHz, CDCl₃) δ=4.00 (dd, J=2.1, 11.6 Hz, 1H), 3.91-3.76 (m, 2H), 3.66-3.46 (m, 3H), 3.13 (d, J=11.5 Hz, 1H), 0.94-0.73 (m, 2H), 0.67-0.49 (m, 2H).

### Example 335: (4-(4,7-dioxaspiro[2.5]octan-5-ylmethoxy)phenyl)-2-oxo-6-(trifluoromethyl)-1,2-dihydropyridine-3-carboxamide (SFC Peak 2) (335)

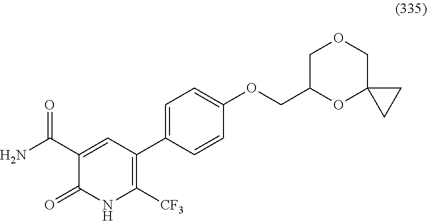 [SMILES: NC(=O)c1cc(-c2ccc(OCC3COCC4(CC4)O3)cc2)c(C(F)(F)F)[nH]c1=O]

(4-(4,7-dioxaspiro[2.5]octan-5-ylmethoxy)phenyl)-2-oxo-6-(trifluoromethyl)-1,2-dihydropyridine-3-carboxamide (SFC peak 2) (335) was obtained by SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4,7-dioxaspiro[2.5]octan-5-ylmethanol. ¹H NMR (400 MHz, MeOD-d4) δ=8.07 (s, 1H), 7.18 (d, J=8.5 Hz, 2H), 6.98-6.88 (m, 2H), 4.19-4.10 (m, 1H), 4.06-3.93 (m, 4H), 3.64-3.59 (m, 1H), 3.25 (d, J=11.5 Hz, 1H), 0.92-0.78 (m, 2H), 0.71-0.58 (m, 2H). 19F NMR (376 MHz, MeOD-d4) δ=−63.04 (s, 3F). LCMS: Rt=0.76 min, m/z=424.9 (M+H). Chiral HPLC: Rt=1.90 min, ee value 87%.

### Example 336: 5-(4-((5,5-difluorotetrahydro-2H-pyran-3-yl)methoxy)phenyl)-2-oxo-6-(trifluoromethyl)-1,2-dihydropyridine-3-carboxamide (SFC Peak 1) (336)

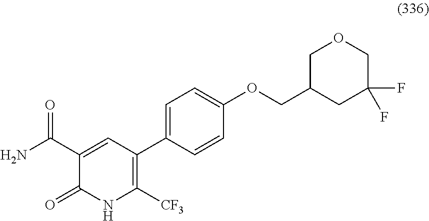 [SMILES: NC(=O)c1cc(-c2ccc(OCC3COCC(F)(F)C3)cc2)c(C(F)(F)F)[nH]c1=O]

5-(4-((5,5-difluorotetrahydro-2H-pyran-3-yl)methoxy)phenyl)-2-oxo-6-(trifluoromethyl)-1,2-dihydropyridine-3-carboxamide (SFC peak 1) was obtained by SFC purification of the product made using a procedure similar to that used to make 5-(4-((1-acetylpiperidin-4-ylmethoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (R)-(5,5-difluorotetrahydro-2H-pyran-3-yl)methanol. ¹H NMR: (400 MHz, DMSO-d6), δ=8.69 (d, J=2.0 Hz, 1H), 8.23 (s, 1H), 8.06 (s, 1H), 7.27 (d, J=8.6 Hz, 2H), 7.03 (d, J=8.6 Hz, 2H), 4.08-3.91 (m, 3H), 3.90-3.78 (m, 1H), 3.68-3.53 (m, 1H), 3.50-3.43 (m, 1H), 2.43-2.27 (m, 2H), 2.12-1.91 (m, 1H). LCMS: Rt=0.76 min, m/z=433.1 (M+H). Chiral HPLC: Rt=1.14 min, ee value=100%.

### (R)-(5,5-difluorotetrahydro-2H-pyran-3-yl)methanol was Obtained Using the Following Procedure

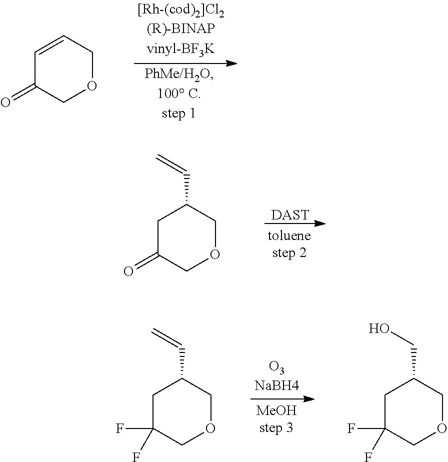

Step 1: To a solution of 2H-pyran-3(6H)-one (10 g, 100 mmol), vinyl-BF₃K (53.6 g, 400 mmol), (R)-BINAP (1.8 g, 3 mmol) in toluene:H₂O=4:1 (200 mL) was added [Rh(cod)C]₂ (1.5 g, 3 mmol) at 25° C. Then the mixture was added stirred at 100° C. for 8 hrs. The reaction mixture was poured into water (500 mL) and the resulting mixture extracted with EA (500 mL×2). The organic layers were combined, dried over anhydrous Na₂SO₄ and concentrated to give crude product which was purified by column chromatography (PE:EA=3:1) to give t(R)-5-vinyldihydro-2H-pyran-3(4H)-one. ¹H NMR: (400 MHz, CDCl₃), δ=5.76 (m, 1H), 5.22-5.04 (m, 2H), 4.10-3.89 (m, 3H), 3.54 (m, 1H), 3.00-2.81 (m, 1H), 2.68 (m, 1H), 2.40 (m, 1H).

Step 2: To a solution of (R)-5-vinyldihydro-2H-pyran-3(4H)-one (2.8 g, 22.2 mmol) in toluene (30 mL) was added DAST (14.7 g, 66.6 mmol) at 0° C. and the mixture stirred at 80° C. for 5 hrs. The reaction mixture was poured into water (50 mL) and the resulting mixture extracted with EA (50 mL×2). The organic layers were combined, dried over anhydrous Na₂SO₄ and concentrated to give crude product which was purified by column chromatography (PE:EA=10:1) to give (R)-3,3-difluoro-5-vinyltetrahydro-2H-pyran. ¹H NMR: (400 MHz, CDCl₃), δ=5.57 (m, 1H), 5.09-5.03 (m, 2H), 3.91-3.78 (m, 2H), 3.45-3.24 (m, 1H), 3.06 (m, 1H), 2.63 (m, 1H), 2.27-2.19 (m, 1H), 1.76-1.58 (m, 1H).

Step 3: To a solution of (R)-3,3-difluoro-5-vinyltetrahydro-2H-pyran (700 mg, 4.73 mmol) in MeOH (10 mL) was stirred at −78° C. under 03 (15 psi) for 1 hr. The mixture was warmed at 0° C. then NaBH4 (215 mg, 5.67 mmol) was added and the mixture stirred for 1 hr. The reaction mixture was concentrated to give crude product which was purified by column chromatography (PE:EA=10:1) to give (R)-(5,5-difluorotetrahydro-2H-pyran-3-yl)methanol.

### Example 337: 5-(4-((5,5-difluorotetrahydro-2H-pyran-3-yl)methoxy)phenyl)-2-oxo-6-(trifluoromethyl)-1,2-dihydropyridine-3-carboxamide (SFC Peak 2) (337)

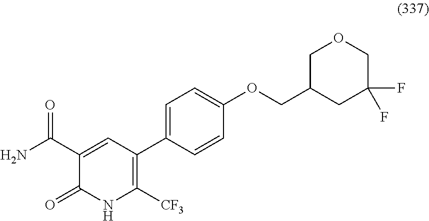 [SMILES: NC(=O)c1cc(-c2ccc(OCC3COCC(F)(F)C3)cc2)c(C(F)(F)F)[nH]c1=O]

5-(4-((5,5-difluorotetrahydro-2H-pyran-3-yl)methoxy)phenyl)-2-oxo-6-(trifluoromethyl)-1,2-dihydropyridine-3-carboxamide (SFC peak 2) (337) was obtained by SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (R)-(5,5-difluorotetrahydro-2H-pyran-3-yl)methanol. ¹H NMR: (400 MHz, DMSO-d6), δ=10.87 (d, J=6.0 Hz, 1H), 7.79 (s, 1H), 7.12 (d, J=8.6 Hz, 2H), 6.98-6.89 (m, 3H), 4.04-3.90 (m, 3H), 3.89-3.75 (m, 1H), 3.70-3.53 (m, 1H), 3.45 (m, 1H), 2.41-2.26 (m, 2H), 2.12-1.91 (m, 1H). LCMS: Rt=0.76 min, m/z=433.1 (M+H). Chiral HPLC: Rt=1.22 min, ee value=93%.

### Example 338: 5-(4-((4,7-dioxaspiro[2.5]octan-6-yl)methoxy)phenyl)-2-oxo-6-(trifluoromethyl)-1,2-dihydropyridine-3-carboxamide (racemate) (338)

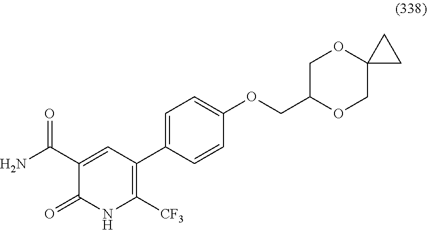 [SMILES: NC(=O)c1cc(-c2ccc(OCC3COC4(CC4)CO3)cc2)c(C(F)(F)F)[nH]c1=O]

5-(4-((4,7-dioxaspiro[2.5]octan-6-yl)methoxy)phenyl)-2-oxo-6-(trifluoromethyl)-1,2-dihydropyridine-3-carboxamide (racemate) (338) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (4,7-dioxaspiro[2.5]octan-6-yl)methanol. ¹H NMR (400 MHz, DMSO-d6) δ=13.59 (s, 1H), 8.66 (s, 1H), 8.23 (s, 1H), 8.06 (s, 1H), 7.31-7.22 (m, 2H), 7.09-6.97 (m, 2H), 4.08-4.05 (m, 2H), 4.05-4.00 (m, 1H), 3.96 (ddd, J=5.0, 7.6, 10.0 Hz, 1H), 3.84 (dd, J=2.5, 11.2 Hz, 1H), 3.61-3.53 (m, 1H), 0.78-0.72 (m, 1H), 0.71-0.65 (m, 1H), 0.61-0.57 (m, 2H). LCMS: Rt=0.77 min, m/z=425.1 (M+H).

### (4,7-dioxaspiro[2.5]octan-6-yl)methanol was Obtained Using the Following Procedure

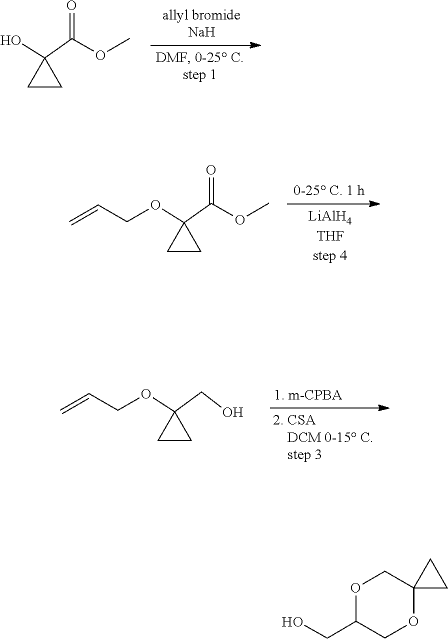

Step 1: To a solution of methyl 1-hydroxycyclopropanecarboxylate (10.0 g, 86.12 mmol) in THF (300 mL) was added NaH (5.17 g, 129.18 mmol) at 0° C. in batches. The mixture was stirred at 0° C. for 1 h. Allyl bromide (15.6 g, 129.18 mmol) was added to the mixture dropwise and the resulting mixture stirred for another 11 hrs. The reaction mixture was poured into water (300 mL) and extracted with EA (200 mL×3). The organic layers were combined, dried over anhydrous Na₂SO₄, filtered and concentrated to give crude product which was purified by silica gel column chromatography (PE) to give methyl 1-(allyloxy)cyclopropane-1-carboxylate. ¹H NMR (400 MHz, CDCl₃) δ=6.06-5.88 (m, 1H), 5.29 (qd, J=1.6, 17.2 Hz, 1H), 5.18 (dd, J=1.4, 10.4 Hz, 1H), 4.15 (m, 2H), 3.77 (s, 3H), 1.34-1.31 (m, 2H), 1.24-1.21 (m, 2H).

Step 2: To a solution of methyl 1-(allyloxy)cyclopropane-1-carboxylate (3.7 g, 23.68 mmol) in THF (50 mL) was added LiAlH₄ (1.35 g, 35.54 mmol) portionwise at 0° C. The reaction mixture was stirred at 25° C. for 1 hr. The reaction mixture was quenched with water (1.35 mL), NaOH (1.35 g) in water (1.35 ml) and water (1.35 mL) in this order. The mixture was stirred for half an hour and filtered by celite and concentrated to give crude product which was purified by silica gel column chromatography (PE/EA=5/1) to give (1-(allyloxy)cyclopropyl)methanol. ¹H NMR (400 MHz, CDCl₃) δ=5.95-5.77 (m, 1H), 5.26-5.17 (m, 1H), 5.08 (dd, J=1.3, 10.4 Hz, 1H), 4.01 (td, J=1.4, 5.4 Hz, 2H), 3.60 (d, J=3.5 Hz, 2H), 0.86-0.80 (m, 2H), 0.57-0.48 (m, 2H).

Step 3: To a solution of (1-(allyloxy)cyclopropyl)methanol (1.0 g, 7.80 mmol) in DCM (10 mL) was added m-CPBA (2.06 g, 10.14 mmol) in one portion at 0° C. The mixture was stirred at 15° C. for 12 hrs. The formed solid was filtered out. To the resulting solution was added CSA (544 mg, 2.34 mmol). The crude product was purified by silica gel column chromatograph (PE/EA=5/1) to give (4,7-dioxaspiro[2.5]octan-6-yl)methanol. ¹H NMR (400 MHz, CDCl₃) δ=4.20 (dd, J=2.2, 11.6 Hz, 1H), 3.86-3.78 (m, 1H), 3.77-3.68 (m, 2H), 3.67-3.61 (m, 2H), 3.27 (d, J=11.6 Hz, 1H), 0.95-0.76 (m, 2H), 0.68-0.54 (m, 2H).

### Example 339: 5-(4-((5,5-dimethyl-1,4-dioxan-2-yl)methoxy)phenyl)-2-oxo-6-(trifluoromethyl)-1,2-dihydropyridine-3-carboxamide (racemate) (339)

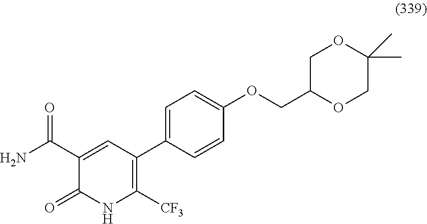 [SMILES: CC1(C)COC(COc2ccc(-c3cc(C(N)=O)c(=O)[nH]c3C(F)(F)F)cc2)CO1]

5-(4-((5,5-dimethyl-1,4-dioxan-2-yl)methoxy)phenyl)-2-oxo-6-(trifluoromethyl)-1,2-dihydropyridine-3-carboxamide (racemate) (339) was made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5,5-dimethyl-1,4-dioxan-2-yl)methanol. ¹H NMR (400 MHz, DMSO-d6) δ=8.23 (s, 1H), 7.94 (s, 1H), 7.16 (d, J=8.4 Hz, 2H), 6.95 (d, J=8.4 Hz, 2H), 3.98 (m, 2H), 3.78 (d, J=4.0 Hz, 1H), 3.64 (m, 1H), 3.60 (m, 1H), 3.54 (d, J=11.4 Hz, 1H), 3.31 (d, J=11.4 Hz, 1H), 1.23 (s, 3H), 1.03 (s, 3H). LCMS: Rt=0.72 min, m/z=427.1 (M+H).

(5,5-dimethyl-1,4-dioxan-2-yl)methano was obtained using the procedure for (4,7-dioxaspiro[2.5]octan-6-yl)methanol, except methyl 1-hydroxycyclopropanecarboxylate was replaced with 2-hydroxy-2-methylpropanoate.

### Example 340: 5-(4-((5-(fluoromethyl)-1,4-dioxan-2-yl)methoxy)phenyl)-2-oxo-6-(trifluoromethyl)-1,2-dihydropyridine-3-carboxamide (SFC Peak 1) (340)

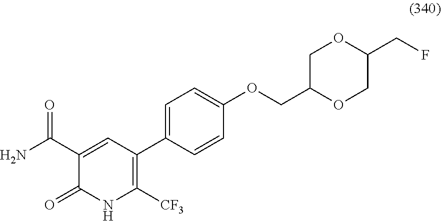 [SMILES: NC(=O)c1cc(-c2ccc(OCC3COC(CF)CO3)cc2)c(C(F)(F)F)[nH]c1=O]

5-(4-((5-(fluoromethyl)-1,4-dioxan-2-yl)methoxy)phenyl)-2-oxo-6-(trifluoromethyl)-1,2-dihydropyridine-3-carboxamide (SFC peak 1) was obtained by SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-(fluoromethyl)-1,4-dioxan-2-yl)methanol. ¹H NMR: (400 MHz, MeOD-d4) δ=8.28 (s, 1H), 7.24 (d, J=8.6 Hz, 2H), 7.01 (d, J=8.8 Hz, 2H), 4.59 (br s, 1H), 4.48 (m, 1H), 4.36 (m, 1H), 4.10-3.98 (m, 3H), 3.97-3.89 (m, 2H), 3.87-3.75 (m, 1H), 3.62 (m, 2H), 1.36-1.26 (m, 1H). 19F NMR (377 MHz, MeOD-d4) δ=−58.68-−66.13 (m, 3F), −235.36 (s, 1F); LCMS: Rt=0.69 min, m/z=431.2 (M+H). Chiral HPLC: Rt=2.77 min, ee value=92.8%.

### (5-(fluoromethyl)-1,4-dioxan-2-yl)methanol was Obtained Using the Following Procedure

Step 1: To a solution of (5-((benzyloxy)methyl)-1,4-dioxan-2-yl)methanol (see Eur. J. Org. Chem., 2001, 5, 875-896) (2 g, 8.39 mmol) in toluene (35 mL) was added DAST (4.06 g, 25.17 mmol) at 0° C. The resulting mixture was stirred at 60° C. for 4 hrs. The mixture was added dropwise to ice water (100 mL) 0° C., diluted with EA (30 mL×3), washed with saturated NaCl solution (2×50 mL), dried over anhydrous Na₂SO₄, filtered and concentrated. The residue was purified by column chromatography (PE/EA=20/1 to 5/1) to give 2-((benzyloxy)methyl)-5-(fluoromethyl)-1,4-dioxane. ¹H NMR (400 MHz, CDCl₃) δ=7.40-7.28 (m, 5H), 4.61-4.53 (m, 2H), 4.49-4.32 (m, 1H), 3.94-3.39 (m, 8H).

To a solution of 2-((benzyloxy)methyl)-5-(fluoromethyl)-1,4-dioxane (1 g, 4.16 mmol) in MeOH (20 mL) was added Pd(OH)₂ (300 mg, 0.21 mmol) at 15° C. The resulting mixture was stirred under 50 psi of H₂ at 50° C. for 12 hrs. The mixture was filtrated and concentrated to give (5-(fluoromethyl)-1,4-dioxan-2-yl)methanol. ¹H NMR (400 MHz, CDCl₃) δ=4.53-4.43 (m, 1H), 4.35 (m, 1H), 4.03-3.40 (m, 12H).

### Example 341: 5-(4-((5-(fluoromethyl)-1,4-dioxan-2-yl)methoxy)phenyl)-2-oxo-6-(trifluoromethyl)-1,2-dihydropyridine-3-carboxamide (SFC Peak 2) (341)

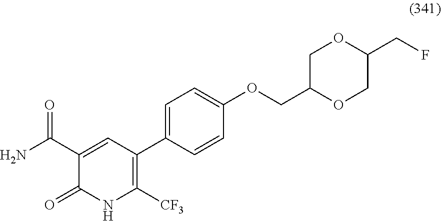 [SMILES: NC(=O)c1cc(-c2ccc(OCC3COC(CF)CO3)cc2)c(C(F)(F)F)[nH]c1=O]

5-(4-((5-(fluoromethyl)-1,4-dioxan-2-yl)methoxy)phenyl)-2-oxo-6-(trifluoromethyl)-1,2-dihydropyridine-3-carboxamide (SFC peak 2) (341) was obtained by SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-(fluoromethyl)-1,4-dioxan-2-yl)methanol. ¹H NMR: (400 MHz, MeOD-d4) δ=8.26 (br s, 1H), 7.24 (br d, J=8.4 Hz, 2H), 7.00 (br d, J=8.0 Hz, 2H), 4.59 (br s, 1H), 4.48 (m, 1H), 4.36 (m, 1H), 4.09-3.98 (m, 3H), 3.93 (m, 2H), 3.87-3.74 (m, 1H), 3.62 (m, 2H), 1.42-1.20 (m, 3H), 0.10 (s, 1H). 19F NMR (377 MHz, MeOD-d4) δ=−58.08-−67.02 (m, 3F), −232.46-−238.72 (m, 1F); LCMS: Rt=0.69 min, m/z=431.2 (M+H). Chiral HPLC: Rt=2.91 min, ee value=95.8%.

### Example 342: 5-(4-((5-(fluoromethyl)-1,4-dioxan-2-yl)methoxy)phenyl)-2-oxo-6-(trifluoromethyl)-1,2-dihydropyridine-3-carboxamide (SFC Peak 3) (342)

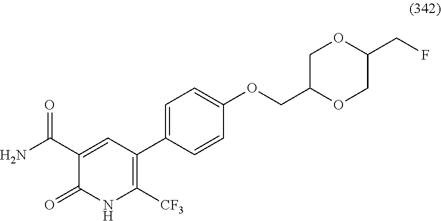 [SMILES: NC(=O)c1cc(-c2ccc(OCC3COC(CF)CO3)cc2)c(C(F)(F)F)[nH]c1=O]

5-(4-((5-(fluoromethyl)-1,4-dioxan-2-yl)methoxy)phenyl)-2-oxo-6-(trifluoromethyl)-1,2-dihydropyridine-3-carboxamide (SFC peak 3) (342) was obtained by SFC purification of the product made using a procedure similar to that used to make 5-(4-((1-acetylpiperidin-4-ylmethoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-(fluoromethyl)-1,4-dioxan-2-yl)methanol. ¹H NMR: (400 MHz, MeOD-5 d4) δ=8.20 (br s, 1H), 7.52-7.31 (m, 1H), 7.21 (br d, J=8.4 Hz, 2H), 6.98 (br d, J=8.4 Hz, 2H), 4.59 (br s, 1H), 4.51-4.31 (m, 2H), 4.11-3.97 (m, 3H), 3.93 (m, 2H), 3.86-3.74 (m, 1H), 3.62 (m, 2H), 1.47-1.06 (m, 5H), 0.10 (s, 1H). 19F NMR (377 MHz, MeOD-d4) δ=−62.92 (br s, 3F), −235.35 (s, 1F). LCMS: Rt=0.69 min, m/z=431.2 (M+H). Chiral HPLC: Rt=3.88 min, ee value=94.6.

### Example 343: 5-(4-((5-(fluoromethyl)-1,4-dioxan-2-yl)methoxy)phenyl)-2-oxo-6-(trifluoromethyl)-1,2-dihydropyridine-3-carboxamide (SFC Peak 4) (342)

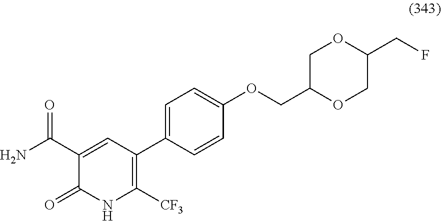 [SMILES: NC(=O)c1cc(-c2ccc(OCC3COC(CF)CO3)cc2)c(C(F)(F)F)[nH]c1=O]

5-(4-((5-(fluoromethyl)-1,4-dioxan-2-yl)methoxy)phenyl)-2-oxo-6-(trifluoromethyl)-1,2-dihydropyridine-3-carboxamide (SFC peak 4) (343) was obtained by SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-(fluoromethyl)-1,4-dioxan-2-yl)methanol. ¹H NMR: (400 MHz, MeOD-d4) δ=8.21 (br s, 1H), 7.53-7.30 (m, 1H), 7.21 (d, J=8.4 Hz, 2H), 6.98 (d, J=8.4 Hz, 2H), 4.59 (br s, 1H), 4.51-4.31 (m, 2H), 4.11-3.97 (m, 3H), 3.97-3.88 (m, 2H), 3.87-3.74 (m, 1H), 3.62 (m, 2H), 1.29 (s, 2H). 19F NMR (377 MHz, MeOD-d4) δ=−62.94 (br s, 3F), −235.35 (s, 1F). LCMS: Rt=0.70 min, m/z=431.2 (M+H). Chiral HPLC: Rt=4.16 min, ee value=90.3%.

### Example 344: 5-(4-((5-(difluoromethyl)-1,4-dioxan-2-yl)methoxy)phenyl)-2-oxo-6-(trifluoromethyl)-1,2-dihydropyridine-3-carboxamide (SFC Peak 1) (344)

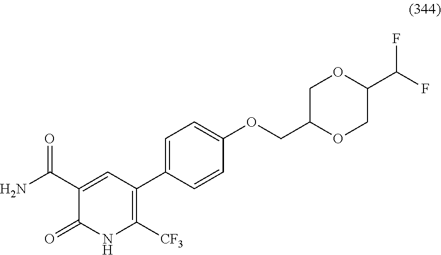 [SMILES: NC(=O)c1cc(-c2ccc(OCC3COC(C(F)F)CO3)cc2)c(C(F)(F)F)[nH]c1=O]

5-(4-((5-(difluoromethyl)-1,4-dioxan-2-yl)methoxy)phenyl)-2-oxo-6-(trifluoromethyl)-1,2-dihydropyridine-3-carboxamide (SFC peak 1) (344) was obtained by SFC purification of the product made using a procedure similar to that used to make 5-(4-((1-acetylpiperidin-4-ylmethoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-(difluoromethyl)-1,4-dioxan-2-yl)methanol. ¹H NMR (400 MHz, DMSO-d6) δ=8.61 (br s, 1H), 8.23 (br s, 1H), 8.08 (br s, 1H), 7.26 (br d, J=8.4 Hz, 2H), 7.04 (d, J=8.4 Hz, 2H), 6.53-6.18 (m, 1H), 4.20-4.14 (m, 1H), 4.13-4.07 (m, 1H), 4.05-3.96 (m, 1H), 3.92-3.85 (m, 1H), 3.85-3.73 (m, 4H). 19F NMR (376 MHz, DMSO-d6) δ=−40.41 (br s, 3F), −126.70-−130.97 (m, 2F). LCMS: Rt=0.72 min, m/z=449.2 (M+H). Chiral HPLC: R=t 3.17 min, de value=95.5%.

### (5-(difluoromethyl)-1,4-dioxan-2-yl)methanol was Obtained Using the Following Procedure

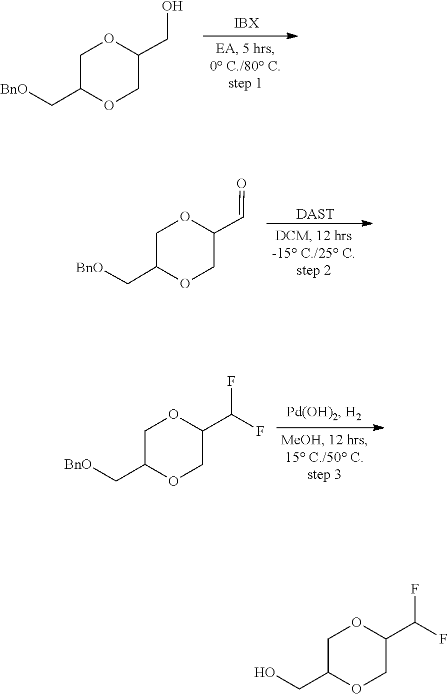

Step 1: To a solution of (5-((benzyloxy)methyl)-1,4-dioxan-2-yl)methanol (6 g, 25.2 mmol, 1.0 eq) in EA (30 mL) was added IBX (17.64 g, 63 mmol, 2.5 eq) at 0° C. The mixture was stirred at 80° C. for 5 hrs. The reaction mixture was filtered and concentrated. The residue was purified by column chromatography (PE/EA=1/1) to give 5-((benzyloxy)methyl)-1,4-dioxane-2-carbaldehyde. ¹H NMR (400 MHz, CDCl₃): δ 10.04-9.48 (m, 1H), 7.45-7.16 (m, 5H), 4.64-4.44 (m, 2H), 4.15-3.12 (m, 8H).

Step 2: To a solution of 5-((benzyloxy)methyl)-1,4-dioxane-2-carbaldehyde (5 g, 21.1 mmol, 1.0 eq) in DCM (200 mL) was added diethylaminosulfur trifluoride (8.5 g, 52.75 mmol, 2.5 eq) at −15° C. The resulting mixture was stirred at 25° C. for 12 hrs. The mixture was washed with brine (200 ml) and extracted with DCM (50 mL×3). The combined organic layers were dried over anhydrous Na₂SO₄, filtered and concentrated. The residue was purified by column chromatography (PE/EA=5/1) to give 2-((benzyloxy)methyl)-5-(difluoromethyl))-1,4-dioxane. ¹H NMR (400 MHz, CDCl₃₊): δ 7.31-7.19 (m, 5H), 6.17-5.78 (m, 1H), 5.76-5.43 (m, 1H), 4.53-4.37 (m, 2H), 3.95-3.20 (m, 8H).

Step 3: To a solution of 2-((benzyloxy)methyl)-5-(difluoromethyl)-1,4-dioxane (1.57 g, 6.08 mmol) in MeOH (30 mL) was added Pd(OH)₂ (427 mg) at 15° C. The resulting mixture was stirred under 50 psi of H₂ at 50° C. for 12 hrs. TLC (PE/EA=3/1, Rf=0.6) showed the starting material was consumed. The mixture was filtrated and concentrated to give (5-(difluoromethyl)-1,4-dioxan-2-yl)methanol. ¹H NMR (400 MHz, CDCl₃) δ=6.27-5.53 (m, 1H), 4.06-3.52 (m, 9H).

### Example 345: 5-(4-((5-(difluoromethyl)-1,4-dioxan-2-yl)methoxy)phenyl)-2-oxo-6-(trifluoromethyl)-1,2-dihydropyridine-3-carboxamide (SFC Peak 2) (345)

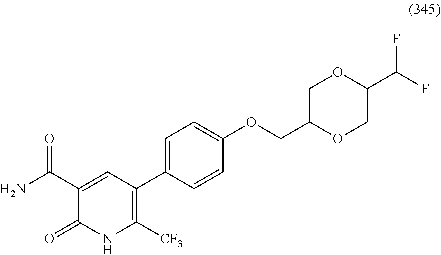 [SMILES: NC(=O)c1cc(-c2ccc(OCC3COC(C(F)F)CO3)cc2)c(C(F)(F)F)[nH]c1=O]

5-(4-((5-(difluoromethyl)-1,4-dioxan-2-yl)methoxy)phenyl)-2-oxo-6-(trifluoromethyl)-1,2-dihydropyridine-3-carboxamide (SFC peak 2) (345) was obtained by SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-(fluoromethyl)-1,4-dioxan-2-yl)methanol. ¹H NMR (400 MHz, DMSO-d6) δ=8.54 (br s, 1H), 8.25 (s, 1H), 8.12 (br s, 1H), 7.27 (br d, J=8.8 Hz, 2H), 7.05 (d, J=8.4 Hz, 2H), 6.57-6.17 (m, 1H), 4.21-4.14 (m, 1H), 4.14-4.07 (m, 1H), 4.05-3.97 (m, 1H), 3.92-3.85 (m, 1H), 3.85-3.73 (m, 4H). 19F NMR (376 MHz, DMSO-d6) δ=−60.42 (br s, 3F), −125.52-−131.88 (m, 2F). LCMS: Rt=0.72 min, m/z=449.2 (M+H). Chiral HPLC: Rt=3.37 min, de value=74.7%.

### Example 346: 5-(4-((5-(difluoromethyl)-1,4-dioxan-2-yl)methoxy)phenyl)-2-oxo-6-(trifluoromethyl)-1,2-dihydropyridine-3-carboxamide (SFC Peak 3) (346)

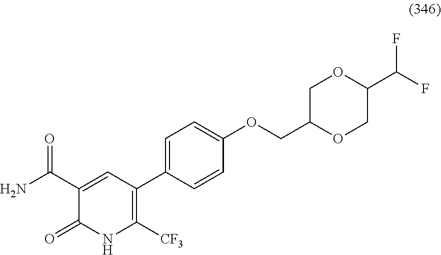 [SMILES: NC(=O)c1cc(-c2ccc(OCC3COC(C(F)F)CO3)cc2)c(C(F)(F)F)[nH]c1=O]

5-(4-((5-(difluoromethyl)-1,4-dioxan-2-yl)methoxy)phenyl)-2-oxo-6-(trifluoromethyl)-1,2-dihydropyridine-3-carboxamide (SFC peak 3) (346) was obtained by SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-(fluoromethyl)-1,4-dioxan-2-yl)methanol. ¹H NMR (400 MHz, DMSO-d6) δ=8.61 (br s, 1H), 8.24 (s, 1H), 8.08 (br s, 1H), 7.26 (br d, J=8.8 Hz, 2H), 7.02 (d, J=8.8 Hz, 2H), 6.22-5.87 (m, 1H), 4.26-4.08 (m, 1H), 4.03 (m, 2H), 4.00-3.92 (m, 1H), 3.92-3.79 (m, 2H), 3.56 (m, 3H). 19F NMR (376 MHz, DMSO-d6) δ=−60.40 (br s, 3F), −101.30-−105.25 (m, 1F), −127.38-−132.46 (m, 1F). LCMS: Rt=0.74 min, m/z=449.2 (M+H). Chiral HPLC: Rt=3.59 min, de value=99.2%.

### Example 347: 5-(4-((5-(difluoromethyl)-1,4-dioxan-2-yl)methoxy)phenyl)-2-oxo-6-(trifluoromethyl)-1,2-dihydropyridine-3-carboxamide (SFC Peak 4) (347)

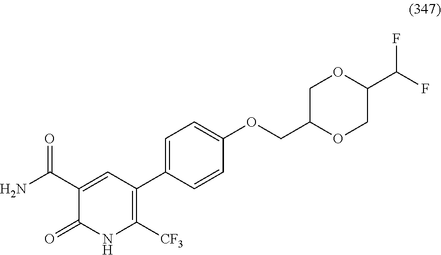 [SMILES: NC(=O)c1cc(-c2ccc(OCC3COC(C(F)F)CO3)cc2)c(C(F)(F)F)[nH]c1=O]

5-(4-((5-(difluoromethyl)-1,4-dioxan-2-yl)methoxy)phenyl)-2-oxo-6-(trifluoromethyl)-1,2-dihydropyridine-3-carboxamide (SFC peak 4) (347) was obtained by SFC purification of the product made using a procedure similar to that used to make 5-(4-((1-acetylpiperidin-4-yl)methoxy)phenyl)-2-oxo-6-(trifluoromethyl)-1,2-dihydropyridine-3-carboxamide (55), except 1-(4-(hydroxymethyl)piperidin-1-yl)ethan-1-one was replaced with (5-(fluoromethyl)-1,4-dioxan-2-yl)methanol. ¹H NMR (400 MHz, DMSO-d6) δ=8.64 (br s, 1H), 8.22 (br s, 1H), 8.06 (br s, 1H), 7.25 (br d, J=8.4 Hz, 2H), 7.02 (d, J=8.8 Hz, 2H), 6.23-5.86 (m, 1H), 4.07-3.96 (m, 3H), 3.95-3.78 (m, 3H), 3.56 (m, 2H). 19F NMR (376 MHz, DMSO-d6) δ=−60.42 (br s, 3F), −126.72-−134.58 (m, 2F). LCMS: Rt=0.74 min, m/z=449.2 (M+H). Chiral HPLC: Rt=3.81 min, de value=82.8%.

### Example 348: 5-(4-((6-(difluoromethyl)-1,4-dioxan-2-yl)methoxy)phenyl)-2-oxo-6-(trifluoromethyl)-1,2-dihydropyridine-3-carboxamide (Trans, SFC Peak 1) (348)

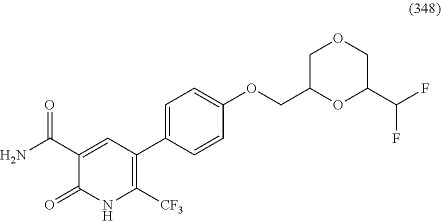 [SMILES: NC(=O)c1cc(-c2ccc(OCC3COCC(C(F)F)O3)cc2)c(C(F)(F)F)[nH]c1=O]

5-(4-((6-(difluoromethyl)-1,4-dioxan-2-yl)methoxy)phenyl)-2-oxo-6-(trifluoromethyl)-1,2-dihydropyridine-3-carboxamide (trans, SFC peak 1) (348) was obtained by SFC purification of the product made using steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 2-((4-bromophenoxy)methyl)-6-(difluoromethyl)-1,4-dioxane. ¹H NMR (400 MHz, DMSO-d6) δ=8.71 (s, 1H), 8.23 (s, 1H), 8.05 (s, 1H), 7.27 (d, J=8.0 Hz, 2H), 7.05 (d, J=8.0 Hz, 2H), 6.54-6.17 (m, 1H), 4.29-4.21 (m, 1H), 4.20-4.10 (m, 2H), 4.08-3.95 (m, 1H), 3.85 (m, 1H), 3.81-3.74 (m, 1H), 3.74-3.68 (m, 1H), 3.64 (m, 1H). LCMS: Rt=0.77 min, m/z=448.9 (M+H). Chiral HPLC: Rt=3.11 min, ee value=100%.

### (6-((4-bromophenoxy)methyl)-1,4-dioxan-2-yl)methanol was Obtained Using the Method Described in Steps 1-4 of Scheme D

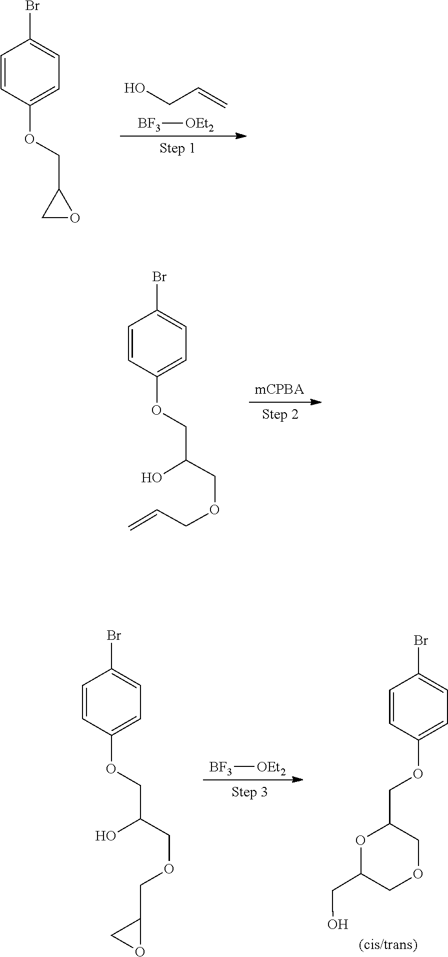

Step 1: To a solution of 2-((4-bromophenoxy)methyl)oxirane (100 g, 436.5 mmol) and dry DCM (Volume: 1.5 L) under N₂ at 0° C. was added prop-2-en-1-ol (45.4 mL, 654.8 mmol, followed by BF₃-diethyl etherate (11 mL, 87.3 mmol). The reaction was stirred at 0° C. for 1 h. Then the reaction was warmed to 25° C. and stirred for 1 h. The mixture was quenched with solid K₂CO₃ (500 g) and MeOH, then stirred at 25° C. for 10 min. The mixture was filtered and concentrated to dryness. The residue was purified by silica gel column (PE to PE/EA=3/1) to afford the title compound as colorless oil (62% yield). 1H NMR (400 MHz, CDCl₃) δ=7.39 (m, 2H), 6.83 (m, 2H), 5.95 (m, 1H), 5.31 (m, 1H), 5.23 (m, 1H), 4.17 (m, 1H), 4.06 (m, 2H), 4.04 (m, 2H), 3.63 (m, 2H), 2.54 (d, J=4.8 Hz, 1H); LCMS: Rt 0.817 min, m/z 287 [M+H].

Step 2: To a solution of 1-(allyloxy)-3-(4-bromophenoxy)propan-2-ol (50 g, 174.13 mmol) and dry DCM (Volume: 400 mL) under N₂ was added m-CPBA (46.0 g, 226.36 mmol. The reaction was stirred at 25° C. for 16 hrs, quenched with 10% (w/v) aqueous Na₂SO₃ and stirred for 30 min, diluted with DCM. The organic layer was separated, washed with saturated aq. NaHCO₃ for three times, dried over sodium sulfate and concentrated. The residue was purified by silica gel column (PE to PE/EA=3/1) to afford the title compound as yellow oil (95% yield). 1H NMR (400 MHz, CDCl₃) δ=7.40 (m, 2H), 6.82 (m, 2H), 4.17 (s, 1H), 4.01 (m, 2H), 3.85 (m, 1H), 3.71 (m, 1H), 3.69 (m, 1H), 3.47 (m, 1H), 3.44 (m 1H), 2.83 (m, 1H), 2.82 (m, 1H), 2.64 (s, 1H), 2.63 (m, 1H).

Step 3: To a solution of 1-(4-bromophenoxy)-3-(oxiran-2-ylmethoxy)propan-2-ol (49.6 g, 164.94 mmol) and dry DCM (3.3 L) under a stream of N₂ was added, BF₃-Et₂O (4.12 mL, 33 mmol. The reaction stirred at 20° C. under N₂ for 3 hrs, quenched with saturated aq. NaHCO₃ (500 mL) and stirred at 20° C. for 5 min. The organic layer was separated. The aqueous layer was extracted aqueous layer with DCM (500 mL×3). The combined organic layers were dried over sodium sulfate, and concentrated. The residue was purified by Combiflash to afford the title compound as a yellow oil. ¹H NMR (400 MHz, CDCl₃) δ=7.39 (d, J=8.8 Hz, 1H), 6.79 (d, J=8.8 Hz, 1H), 4.07 (m, 1H), 4.04-3.99 (m, 1H), 3.96-3.88 (m, 1H), 3.87-3.76 (m, 1H), 3.73-3.65 (m, 1H), 3.63-3.55 (m, 1H), 3.45 (m, 1H); LCMS: Rt 0.73 min, m/z 303, 305 (M+H).

### 2-((4-bromophenoxy)methyl)-6-(difluoromethyl)-1,4-dioxane was Obtained Using the Following Procedure

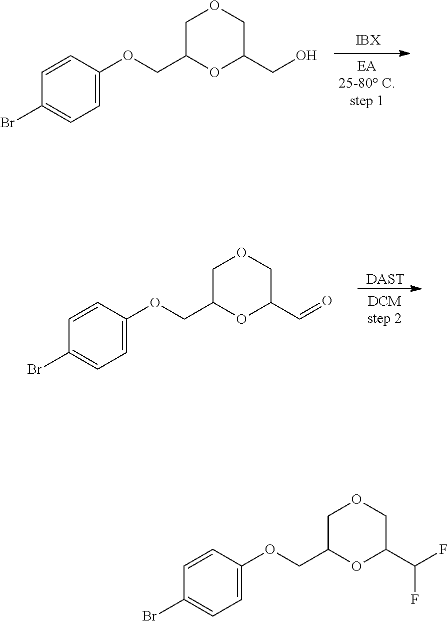

Step 1: To a solution of (6-((4-bromophenoxy)methyl)-1,4-dioxan-2-yl)methanol (1.0 g, 3.30 mmol) in EA (20 mL) was added IBX (2.31 g, 8.25 mmol) at 25° C. Then the mixture was stirred at 80° C. for 3 hr. The reaction mixture was filtered and concentrated to give 6-((4-bromophenoxy)methyl)-1,4-dioxane-2-carbaldehyde which was used for the next step directly. ¹H NMR (400 MHz, CDCl₃) δ=9.89 (s, 1H), 9.63 (s, 1H), 7.40 (d, J=8.0 Hz, 2H), 6.85-6.73 (m, 2H), 4.37-4.30 (m, 1H), 4.28-3.89 (m, 6H), 3.87-3.78 (m, 1H), 3.68-3.59 (m, 1H), 3.53-3.39 (m, 1H).

Step 2: To a solution of 6-((4-bromophenoxy)methyl)-1,4-dioxane-2-carbaldehyde (1.0 g, 3.32 mmol) in anhydrous DCM (10 mL) was added DAST (642 mg, 3.98 mmol) at 0° C. The mixture was stirred at 20° C. for 12 hr. The reaction mixture was quenched with saturated NaHCO₃ solution (3 mL), 10 mL of water was added then extracted with DCM (10 mL×2). The combined organic phase was dried over anhydrous Na₂SO₄, filtered and concentrated to give crude product which was purified by silica gel column chromatography (PE to PE/EA=10/1) to give 2-((4-bromophenoxy)methyl)-6-(difluoromethyl)-1,4-dioxane. ¹H NMR (400 MHz, CDCl₃) δ=7.44-7.36 (m, 2H), 6.85-6.77 (m, 2H), 6.21-5.58 (m, 1H), 4.28-4.19 (m, 1H), 4.16-4.02 (m, 2H), 4.00-3.90 (m, 3H), 3.88-3.84 (m, 1H), 3.73 (m, 1H), 3.60-3.45 (m, 1H).

### Example 349: 5-(4-((6-(difluoromethyl)-1,4-dioxan-2-yl)methoxy)phenyl)-2-oxo-6-(trifluoromethyl)-1,2-dihydropyridine-3-carboxamide (Trans, SFC Peak 2) (349)

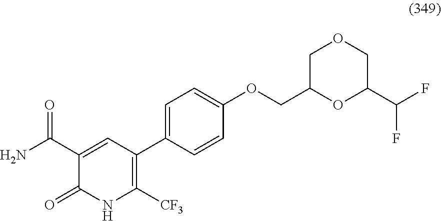 [SMILES: NC(=O)c1cc(-c2ccc(OCC3COCC(C(F)F)O3)cc2)c(C(F)(F)F)[nH]c1=O]

5-(4-((6-(difluoromethyl)-1,4-dioxan-2-yl)methoxy)phenyl)-2-oxo-6-(trifluoromethyl)-1,2-dihydropyridine-3-carboxamide (trans, SFC peak 2) (349) was obtained by SFC purification of the product made using steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 2-((4-bromophenoxy)methyl)-6-(difluoromethyl)-1,4-dioxane. ¹H NMR (400 MHz, DMSO-d6) δ=8.71 (s, 1H), 8.23 (s, 1H), 8.05 (s, 1H), 7.27 (d, J=8.0 Hz, 2H), 7.05 (d, J=8.0 Hz, 2H), 6.54-6.17 (m, 1H), 4.29-4.21 (m, 1H), 4.20-4.10 (m, 2H), 4.08-3.95 (m, 1H), 3.85 (m, 1H), 3.81-3.74 (m, 1H), 3.74-3.68 (m, 1H), 3.64 (m, 1H). LCMS: Rt=0.73 min, m/z=449.2 (M+H). Chiral HPLC: Rt=3.41 min, ee value=100%.

### Example 350: 5-(4-((6-(difluoromethyl)-1,4-dioxan-2-yl)methoxy)phenyl)-2-oxo-6-(trifluoromethyl)-1,2-dihydropyridine-3-carboxamide (Cis Racemic) (350)

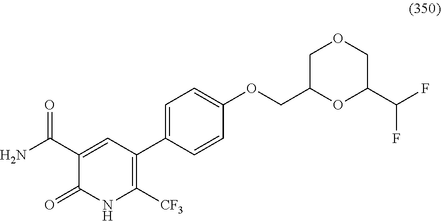 [SMILES: NC(=O)c1cc(-c2ccc(OCC3COCC(C(F)F)O3)cc2)c(C(F)(F)F)[nH]c1=O]

5-(4-((6-(difluoromethyl)-1,4-dioxan-2-yl)methoxy)phenyl)-2-oxo-6-(trifluoromethyl)-1,2-dihydropyridine-3-carboxamide (cis racemic) (350) was made using steps 2 and 3 in the procedure used to make 2-oxo-5-(4-((tetrahydro-2H-pyran-4-yl)methyl)phenyl)-6-(trifluoromethyl)-1,2-dihydropyridine-3-carboxamide (52), except 4-(4-bromobenzylidene)tetrahydro-2H-pyran was replaced with 2-((4-bromophenoxy)methyl)-6-(difluoromethyl)-1,4-dioxane. ¹H NMR (400 MHz, DMSO-d6) δ=13.61 (s, 1H), 8.56 (s, 1H), 8.25 (s, 1H), 8.12 (s, 1H), 7.27 (d, J=8.0 Hz, 2H), 7.04 (d, J=8.0 Hz, 2H), 6.23-5.90 (m, 1H), 4.09-4.01 (m, 4H), 3.94-3.88 (m, 1H), 3.84 (m, 1H), 3.47-3.39 (m, 2H). LCMS: Rt=0.77 min, m/z=449.0 (M+H).

### Example 351: 5-(4-((6-(fluoromethyl)-1,4-dioxan-2-yl)methoxy)phenyl)-2-oxo-6-(trifluoromethyl)-1,2-dihydropyridine-3-carboxamide (Cis, SFC Peak 1) (351)

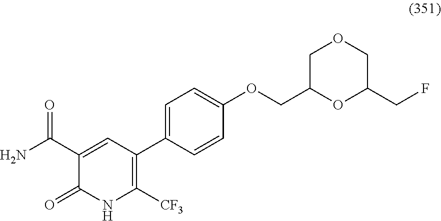 [SMILES: NC(=O)c1cc(-c2ccc(OCC3COCC(CF)O3)cc2)c(C(F)(F)F)[nH]c1=O]

5-(4-((6-(fluoromethyl)-1,4-dioxan-2-yl)methoxy)phenyl)-2-oxo-6-(trifluoromethyl)-1,2-dihydropyridine-3-carboxamide (cis, SFC peak 1) (351) was obtained using the procedure described for Example 347, except 2-((4-bromophenoxy)methyl)-6-(difluoromethyl)-1,4-dioxane was replaced with 2-((4-bromophenoxy)methyl)-6-(fluoromethyl)-1,4-dioxane. ¹H NMR (400 MHz, DMSO-d6) δ=8.14 (s, 1H), 7.23 (d, J=8.0 Hz, 2H), 7.01 (d, J=8.0 Hz, 2H), 4.52-4.50 (m, 1H), 4.41-4.38 (m, 1H), 4.06-3.98 (m, 3H), 3.97-3.91 (m, 1H), 3.90-3.85 (m, 2H), 3.82-3.78 (m, 1H). LCMS: Rt=0.76 min, m/z=431.2 (M+H). Chiral HPLC: Rt=2.69 min, de value=87.51%.

### 2-((4-bromophenoxy)methyl)-6-(fluoromethyl)-1,4-dioxane was Obtained Using the Following Procedure

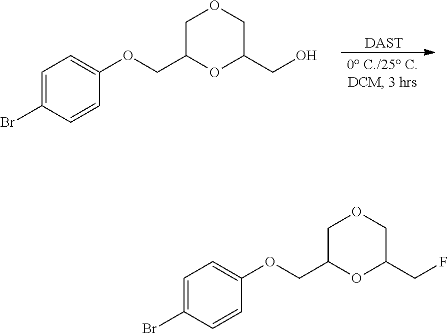

2-((4-bromophenoxy)methyl)-6-(fluoromethyl)-1,4-dioxane was prepared using the procedure in step 2 of the synthesis of 2-((4-bromophenoxy)methyl)-6-(difluoromethyl)-1,4-dioxane, except 6-((4-bromophenoxy)methyl)-1,4-dioxane-2-carbaldehyde was replaced with (6-((4-bromophenoxy)methyl)-1,4-dioxan-2-yl)methanol ¹H NMR (400 MHz, CDCl₃) δ=7.38 (d, J=8.0 Hz, 2H), 6.78 (d, J=8.0 Hz, 2H), 4.50 (d, J=4.0 Hz, 1H), 4.40-4.38 (m, 1H), 4.09-4.05 (m, 1H), 4.04-4.00 (m, 1H), 3.98-3.92 (m, 2H), 3.91-3.88 (m, 1H), 3.87-3.84 (m, 1H), 3.52-3.41 (m, 2H).

### Example 352: 5-(4-((6-(fluoromethyl)-1,4-dioxan-2-yl)methoxy)phenyl)-2-oxo-6-(trifluoromethyl)-1,2-dihydropyridine-3-carboxamide (Cis, SFC Peak 2) (352)

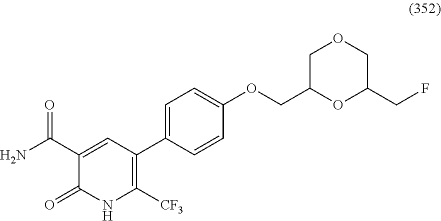 [SMILES: NC(=O)c1cc(-c2ccc(OCC3COCC(CF)O3)cc2)c(C(F)(F)F)[nH]c1=O]

This compound was separated by SFC from the mixture containing Example 351.

5-(4-((6-(fluoromethyl)-1,4-dioxan-2-yl)methoxy)phenyl)-2-oxo-6-(trifluoromethyl)-1,2-dihydropyridine-3-carboxamide (cis, SFC peak 2) (352) was obtained by SFC purification of the product made in Example 351. ¹H NMR (400 MHz, DMSO-d6) δ=8.07 (s, 1H), 7.21 (d, J=8.0 Hz, 2H), 7.00 (d, J=8.0 Hz, 2H), 4.52-4.50 (m, 1H), 4.41-4.38 (m, 1H), 4.05-3.98 (m, 3H), 3.97-3.91 (m, 1H), 3.90-3.84 (m, 2H), 3.81-3.78 (m, 1H). LCMS: Rt=0.76 min, m/z=431.2 (M+H). Chiral HPLC: Rt=2.78 min, de value=86.57%.

### Example 353: 5-(4-((6-(1-methoxycyclopropyl)-1,4-dioxan-2-yl)methoxy)phenyl)-2-oxo-6-(trifluoromethyl)-1,2-dihydropyridine-3-carboxamide (Cis, SFC Peak 1) (353)

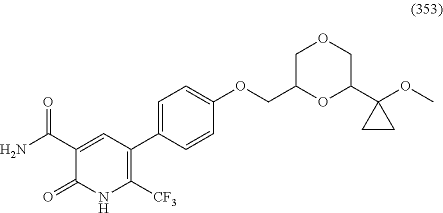 [SMILES: COC1(C2COCC(COc3ccc(-c4cc(C(N)=O)c(=O)[nH]c4C(F)(F)F)cc3)O2)CC1]

5-(4-((6-(1-methoxycyclopropyl)-1,4-dioxan-2-yl)methoxy)phenyl)-2-oxo-6-(trifluoromethyl)-1,2-dihydropyridine-3-carboxamide (cis, SFC peak 1) (353) was obtained using the procedure described for Example 347, except 2-((4-bromophenoxy)methyl)-6-(difluoromethyl)-1,4-dioxane was replaced with 2-((4-bromophenoxy)methyl)-6-(1-methoxycyclopropyl)-1,4-dioxane. 1H NMR: (400 MHz, DMSO) δ=8.11 (s, 1H), 7.21 (d, J=8.0 Hz, 2H), 7.00 (d, J=8.4 Hz, 2H), 4.15-4.07 (m, 1H), 4.06-3.97 (m, 2H), 3.95-3.88 (m, 1H), 3.84 (m, 2H), 3.58 (m, 1H), 3.43-3.38 (m, 3H), 3.27 (s, 3H), 3.16 (m, 3H), 0.74-0.55 (m, 4H). LCMS: Rt=0.77 min, m/z=469.0 (M+H). Chiral HPLC: Rt=3.16 min, ee value=90.46%.

### 2-((4-bromophenoxy)methyl)-6-(1-methoxycyclopropyl)-1,4-dioxane was Obtained Using the Following Procedure

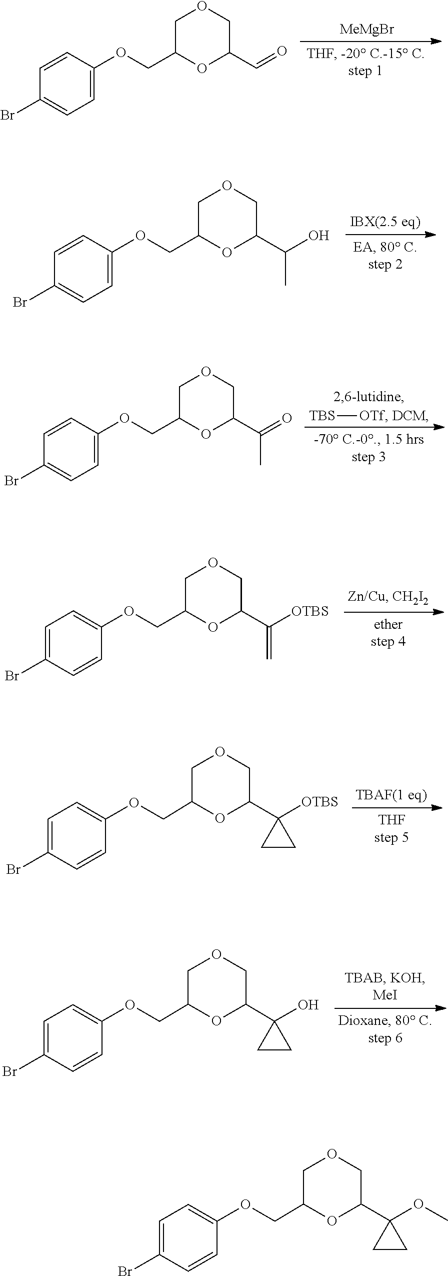

Step 1: To a solution of 6-((4-bromophenoxy)methyl)-1,4-dioxane-2-carbaldehyde (2.5 g, 5.31 mmol) in anhydrous THF (30 mL) was added MeMgBr (8.3 mL, 24.91 mmol, 3 M Et₂O) at −60° C. under N₂. The reaction mixture was warmed to 25° C. and stirred for 2 hrs. The mixture was poured into ice saturated NH₄Cl solution (40 mL) and extracted with EA (30 mL×3). The organic layers were combined, dried over Na₂SO₄ and concentrated in vacuum. The residue was purified by silica gel chromatography (PE/Ethyl acetate=10/1, 1/1) to give 1-(6-((4-bromophenoxy)methyl)-1,4-dioxan-2-yl)ethano. LCMS: Rt=0.77 min, m/z=317, 319 (M+H).

Step 2: 1-(6-((4-bromophenoxy)methyl)-1,4-dioxan-2-yl)ethanone was prepared using the same procedure as 5-((benzyloxy)methyl)-1,4-dioxane-2-carbaldehyde, except (5-((benzyloxy)methyl)-1,4-dioxan-2-yl)methanol was replaced with 1-(6-((4-bromophenoxy)methyl)-1,4-dioxan-2-yl)ethanol. ¹H NMR (400 MHz, CDCl₃) δ=7.43-7.35 (m, 2H), 6.89-6.77 (m, 2H), 4.20 (m, 1H), 4.14-4.02 (m, 3H), 3.99-3.91 (m, 2H), 3.52-3.32 (m, 2H), 2.23 (s, 3H).

Step 3: To a solution of 1-(6-((4-bromophenoxy)methyl)-1,4-dioxan-2-yl)ethanone (2.0 g, 6.35 mmol, 2,6-lutidine (2 mL) in anhydrous DCM (20 mL) was added TBSOTf (2 mL) at −70° C. under N₂. Then the mixture was stirred at 0° C. for 2 hrs. The reaction mixture was poured into aq·NaHCO₃ (20 mL) and the resulting mixture extracted with DCM (10 mL×2). The organic layers were combined, dried over anhydrous Na₂SO₄, filtered and concentrated to give crude product which was purified by silica gel column (PE) to give ((1-(6-((4-bromophenoxy)methyl)-1,4-dioxan-2-yl)vinyl)oxy)(tert-buty)dimethylsilane. ¹H NMR (400 MHz, CDCl₃) δ=7.42-7.35 (m, 2H), 6.84-6.77 (m, 2H), 4.42 (s, 1H), 4.22 (m, 1H), 4.10-4.02 (m, 3H), 4.00-3.87 (m, 3H), 3.49-3.29 (m, 2H), 0.93 (s, 9H), 0.18 (m, 6H).

Step 4: To a solution of ((1-(6-((4-bromophenoxy)methyl)-1,4-dioxan-2-yl)vinyl)oxy)(tert-butyl)dimethylsilane (1.0 g, 2.33 mmol) in anhydrous Et₂O (10 mL) was added in the following order zinc-copper couple (3.05 g, 20.88 mmol), CH₂I₂ (3.12 g, 0.94 mL, 11.64 mmol) and I₂ (1.18 g, 4.66 mmol). The reaction mixture was heated to 45° C. and stirred for 16 hrs. The reaction mixture was poured into water (20 mL) and the mixture extracted with EA (10 mL×2). The organic layers were combined, dried over anhydrous Na2SO4, filtered and concentrated to give crude product which was purified by silica gel column chromatography (PE) to give (1-(6-((4-bromophenoxy)methyl)-1,4-dioxan-2-yl)cyclopropoxy)(tert-buty)dimethylsilane. ¹H NMR (400 MHz, CDCl₃) δ=7.38 (m, 2H), 6.80-6.75 (m, 2H), 4.03-3.83 (m, 7H), 3.63-3.53 (m, 1H), 3.46-3.38 (m, 1H), 3.29 (m, 1H), 0.84 (s, 9H), 0.77-0.54 (m, 4H), 0.15 (s, 3H), 0.10 (s, 3H).

Step 5: To a solution of (1-(6-((4-bromophenoxy)methyl)-1,4-dioxan-2-yl)cyclopropoxy)(tert-butyl)dimethylsilane (160 mg, 0.406 mmol) in anhydrous THF (10 mL) was added TBAF (0.6 mL, 0.609 mmol) at 25° C. The reaction mixture was stirred at 25° C. for 4 hrs. The mixture was poured into water (10 mL) and extracted with EA (5 mL×3). The organic layers were combined, dried over anhydrous Na₂SO₄, filtered and concentrated. The residue was purified by silica gel chromatography (PE/EA=10/1) to give 1-(6-((4-bromophenoxy) methyl)-1,4-dioxan-2-yl)cyclopropanol. LCMS: Rt=0.80 min, m/z=351.0, 353.0 (M+Na).

Step 6: To a solution of 1-(6-((4-bromophenoxy) methyl)-1,4-dioxan-2-yl)cyclopropanol (100 mg, 0.30 mmol) in anhydrous dioxane (3 mL) were added TBAB (98 mg, 0.30 mmol), KOH (130 mg, 1.22 mmol) and MeI (130 mg, 0.91 mmol) at 25° C. and the mixture stirred at 80° C. for 4 hrs. The reaction mixture was diluted with water (5 mL) and extracted with EA (3 mL×3). The organic layers were combined, dried over anhydrous Na₂SO₄, filtered and concentrated. The residue was purified by silica gel chromatography (PE/EA=20/1) to give 2-((4-bromophenoxy)methyl)-6-(1-methoxycyclopropyl)-1,4-dioxane. LCMS: Rt=0.87 min, m/z=365, 367 (M+Na).

### Example 354: 5-(4-((6-(2-hydroxypropan-2-yl)-1,4-dioxan-2-yl)methoxy)phenyl)-2-oxo-6-(trifluoromethyl)-1,2-dihydropyridine-3-carboxamide (Cis, SFC Peak 1) (354)

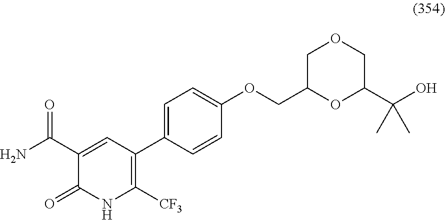 [SMILES: CC(C)(O)C1COCC(COc2ccc(-c3cc(C(N)=O)c(=O)[nH]c3C(F)(F)F)cc2)O1]

5-(4-((6-(2-hydroxypropan-2-yl)-1,4-dioxan-2-yl)methoxy)phenyl)-2-oxo-6-(trifluoromethyl)-1,2-dihydropyridine-3-carboxamide (cis, SFC peak 1) (354) was obtained using the procedure described for Example 348, except 2-((4-bromophenoxy)methyl)-6-(difluoromethyl)-1,4-dioxane was replaced with 2-(6-((4-bromophenoxy)methyl)-1,4-dioxan-2-yl)propan-2-ol. ¹H NMR: (400 MHz, DMSO-d6), δ=8.86-8.55 (m, 1H), 8.24 (s, 1H), 8.07 (br s, 1H), 7.24 (d, J=8.4 Hz, 2H), 7.03 (d, J=8.4 Hz, 2H), 4.44 (s, 1H), 4.03-4.01 (m, 2H), 3.84-3.82 (m, 1H), 3.80-3.79 (m, 2H), 3.33-3.24 (m, 3H), 1.09 (s, 3H), 1.03 (s, 3H). LCMS: Rt=0.73 min, m/z=457.1 (M+H). Chiral HPLC: Rt=1.92 min, ee value=100%.

### Example 355: 5-(4-((6-(2-hydroxypropan-2-yl)-1,4-dioxan-2-yl)methoxy)phenyl)-2-oxo-6-(trifluoromethyl)-1,2-dihydropyridine-3-carboxamide (Cis, SFC Peak 2) (355)

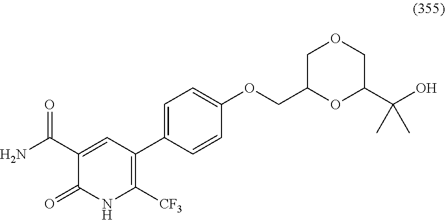 [SMILES: CC(C)(O)C1COCC(COc2ccc(-c3cc(C(N)=O)c(=O)[nH]c3C(F)(F)F)cc2)O1]

5-(4-((6-(2-hydroxypropan-2-yl)-1,4-dioxan-2-yl)methoxy)phenyl)-2-oxo-6-(trifluoromethyl)-1,2-dihydropyridine-3-carboxamide (cis, SFC peak 2) (355) was obtained using the procedure described for Example 348, except 2-((4-bromophenoxy)methyl)-6-(difluoromethyl)-1,4-dioxane was replaced with 2-(6-((4-bromophenoxy)methyl)-1,4-dioxan-2-yl)propan-2-ol. ¹H NMR: (400 MHz, DMSO-d6), δ=8.98-8.62 (m, 1H), 8.26 (s, 1H), 8.08 (br s, 1H), 7.24 (d, J=8.4 Hz, 2H), 7.02 (d, J=8.4 Hz, 2H), 4.46 (s, 1H), 4.03-4.01 (m, 2H), 3.84-3.82 (m, 1H), 3.82-3.79 (m, 2H), 3.35-3.27 (m, 2H), 1.09 (s, 3H), 1.03 (s, 3H). LCMS: Rt=0.73 min, m/z=457.1 (M+H). Chiral HPLC: Rt=2.92 min, ee value=97%.

### Example 356: 5-(4-((6-(1-methylcyclopropyl)-1,4-dioxan-2-yl)methoxy)phenyl)-2-oxo-6-(trifluoromethyl)-1,2-dihydropyridine-3-carboxamide (Cis, SFC Peak 1) (356)

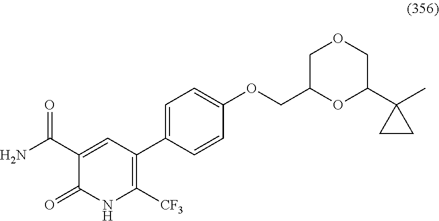 [SMILES: CC1(C2COCC(COc3ccc(-c4cc(C(N)=O)c(=O)[nH]c4C(F)(F)F)cc3)O2)CC1]

5-(4-((6-(1-methylcyclopropyl)-1,4-dioxan-2-yl)methoxy)phenyl)-2-oxo-6-(trifluoromethyl)-1,2-dihydropyridine-3-carboxamide (cis, SFC peak 1) (356) was obtained using the procedure described for Example 348, except 2-((4-bromophenoxy)methyl)-6-(difluoromethyl)-1,4-dioxane was replaced with 2-((4-bromophenoxy)methyl)-6-(1-methylcyclopropyl)-1,4-dioxane. ¹H NMR (400 MHz, DMSO-d6) δ=9.23 (s, 1H), 8.16 (s, 1H), 8.09 (s, 1H), 7.74 (s, 1H), 7.21 (d, J=8.0 Hz, 2H), 7.00 (d, J=8.0 Hz, 2H), 4.06-3.95 (m, 2H), 3.93-3.86 (m, 1H), 3.82 (d, J=12.0 Hz, 1H), 3.74 (d, J=12.0 Hz, 1H), 3.05 (d, J=12.0 Hz, 1H), 0.99 (s, 3H), 0.50-0.43 (m, 2H), 0.27-0.17 (m, 2H). LCMS: Rt=0.84 min, m/z=453.0 (M+H). Chiral HPLC: Rt=3.91 min, de value=95.5%.

### 2-((4-bromophenoxy)methyl)-6-(1-methylcyclopropyl)-1,4-dioxane was Obtained Using the Following Procedure

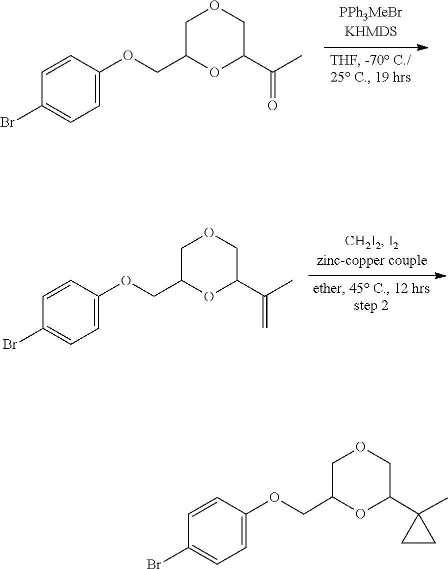 [SMILES: CC(=O)C1COCC(COc2ccc(Br)cc2)O1 ; C=C(C)C1COCC(COc2ccc(Br)cc2)O1 ; CC1(C2COCC(COc3ccc(Br)cc3)O2)CC1]

Step 1: A solution of bromo(methyl)triphenylphosphorane (3.63 g, 10.15 mmol) in dry THF (24 mL) under N₂ at 25° C. was added KHMDS (1M, 10 mL, 10.15 mmol) The mixture was stirred for 1.5 hrs at 25° C., then cooled to −70° C. A solution of 1-(6-((4-bromophenoxy)methyl)-1,4-dioxan-2-yl)ethanone (800 mg, 2.54 mmol) in THF (8 mL) was added dropwise and the reaction mixture was stirred at −70° C. for 2.5 hrs, then allowed to warm to 25° C. and stirred for 15 hrs. The reaction was quenched by the addition of H₂O (50 mL) and extracted with EA (50 mL×3). The organic layers were combined, dried over anhydrous Na₂SO₄, filtered and concentrated in vacuo to give a residue which was purified by column chromatography (PE/EA=20/1) to give 2-((4-bromophenoxy)methyl)-6-(prop-1-en-2-yl)-1,4-dioxane. LCMS: Rt=0.93 min, m/z=313.0 (M+H). 2-((4-bromophenoxy)methyl)-6-(1-methylcyclopropyl)-1,4-dioxane was obtained using the method of step for in the synthesis of 2-((4-bromophenoxy)methyl)-6-(1-methoxycyclopropyl)-1,4-dioxane, except ((1-(6-((4-bromophenoxy)methyl)-1,4-dioxan-2-yl)vinyloxy)(tert-butyl)dimethylsilane was replaced with 2-((4-bromophenoxy)methyl)-6-(prop-1-en-2-yl)-1,4-dioxane. ¹H NMR (400 MHz, CDCl₃) δ=7.42-7.34 (m, 2H), 6.83-6.74 (m, 2H), 4.06-3.99 (m, 1H), 3.99-3.94 (m, 1H), 3.94-3.78 (m, 3H), 3.50-3.29 (m, 2H), 3.08-3.05 (m, 1H), 1.05 (s, 3H), 0.57-0.43 (m, 2H), 0.37-0.23 (m, 2H).

### Example 357: 5-(4-((6-(1-methylcyclopropyl)-1,4-dioxan-2-yl)methoxy)phenyl)-2-oxo-6-(trifluoromethyl)-1,2-dihydropyridine-3-carboxamide (Cis, SFC Peak 2) (357)

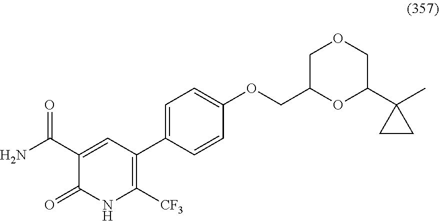 [SMILES: CC1(C2COCC(COc3ccc(-c4cc(C(N)=O)c(=O)[nH]c4C(F)(F)F)cc3)O2)CC1]

5-(4-((6-(1-methylcyclopropyl)-1,4-dioxan-2-yl)methoxy)phenyl)-2-oxo-6-(trifluoromethyl)-1,2-dihydropyridine-3-carboxamide (cis, SFC peak 2) (357) was obtained using the procedure described for Example 348, except 2-((4-bromophenoxy)methyl)-6-(difluoromethyl)-1,4-dioxane was replaced with 2-((4-bromophenoxy)methyl)-6-(1-methylcyclopropyl)-1,4-dioxane. ¹H NMR (400 MHz, DMSO-d6) δ=8.59 (s, 1H), 8.00-7.59 (m, 2H), 7.01 (d, J=8.0 Hz, 2H), 6.80 (d, J=8.0 Hz, 2H), 3.84-3.73 (m, 2H), 3.72-3.63 (m, 1H), 3.59 (d, J=12.0 Hz, 1H), 3.52 (d, J=12.0 Hz, 1H), 2.82 (d, J=12.0 Hz, 1H), 0.76 (s, 3H), 0.27-0.19 (m, 2H), 0.06-0.03 (m, 2H); LCMS: Rt=0.835 min, m/z=453.0 (M+H), Chiral HPLC: Rt=4.258 min, de value=87%.

### Example 358: 5-(4-((6-(1,1-difluoroethyl)-1,4-dioxan-2-yl)methoxy)phenyl)-2-oxo-6-(trifluoromethyl)-1,2-dihydropyridine-3-carboxamide (Cis, SFC Peak 2) (358)

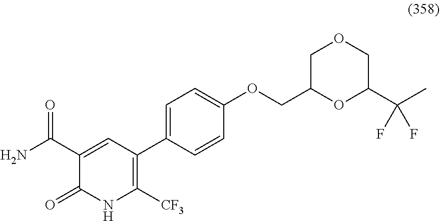 [SMILES: CC(F)(F)C1COCC(COc2ccc(-c3cc(C(N)=O)c(=O)[nH]c3C(F)(F)F)cc2)O1]

5-(4-((6-(1,1-difluoroethyl)-1,4-dioxan-2-yl)methoxy)phenyl)-2-oxo-6-(trifluoromethyl)-1,2-dihydropyridine-3-carboxamide (cis, SFC peak 2) (358) was obtained using the procedure described for Example 348, except 2-((4-bromophenoxy)methyl)-6-(difluoromethyl)-1,4-dioxane was replaced with 2-((4-bromophenoxy)methyl)-6-(1,1-difluoroethyl)-1,4-dioxane. ¹H NMR (400 MHz, MeOD-d4) δ=8.30 (s, 1H), 7.25 (d, J=8.0 Hz, 2H), 7.02 (d, J=8.0 Hz, 2H), 4.16-4.03 (m, 3H), 3.98-3.86 (m, 3H), 3.55-3.46 (m, 2H), 1.62 (t, J=20 Hz, 3H). LCMS: Rt=0.78 min, m/z=463.2 (M+H). Chiral HPLC: Rt=4.12 min, ee value=98%.

### 2-((4-bromophenoxy)methyl)-6-(1,1-difluoroethyl)-1,4-dioxane was Obtained Using the Following Procedure

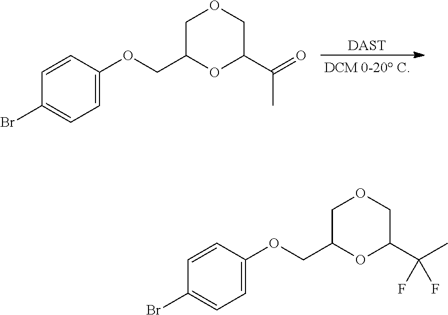

2-((4-bromophenoxy)methyl)-6-(1,1-difluoroethyl)-1,4-dioxane was prepared using the procedure in step 2 of the synthesis of 2-((4-bromophenoxy)methyl)-6-(difluoromethyl)-1,4-dioxane, except 6-((4-bromophenoxy)methyl)-1,4-dioxane-2-carbaldehyde was replaced with 1-(6-((4-bromophenoxy)methyl)-1,4-dioxan-2-yl)ethanone. LCMS: Rt=0.91 min, m/z=337, 339 (M+H).

### Example 359: 5-(4-((6-(1,1-difluoroethyl)-1,4-dioxan-2-yl)methoxy)phenyl)-2-oxo-6-(trifluoromethyl)-1,2-dihydropyridine-3-carboxamide (Cis, SFC Peak 1) (359)

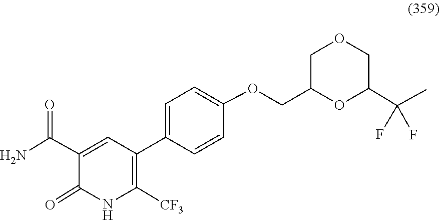 [SMILES: CC(F)(F)C1COCC(COc2ccc(-c3cc(C(N)=O)c(=O)[nH]c3C(F)(F)F)cc2)O1]

5-(4-((6-(1,1-difluoroethyl)-1,4-dioxan-2-yl)methoxy)phenyl)-2-oxo-6-(trifluoromethyl)-1,2-dihydropyridine-3-carboxamide (cis, SFC peak 1) (359) was obtained by SFC purification of the product made in Example 357. ¹H NMR (400 MHz, METHANOL-d4) δ=8.26 (s, 1H), 7.24 (d, J=8.0 Hz, 2H), 7.01 (d, J=8.0 Hz, 2H), 4.15-4.03 (m, 3H), 3.97-3.86 (m, 3H), 3.54-3.46 (m, 2H), 1.62 (t, J=20 Hz, 3H). LCMS: Rt=0.78 min, m/z=463.2 (M+H). Chiral HPLC: Rt=3.04 min, ee value=100%.

### Example 360: 5-(4-((6-(ethoxymethyl)-1,4-dioxan-2-yl)methoxy)phenyl)-2-oxo-6-(trifluoromethyl)-1,2-dihydropyridine-3-carboxamide (Cis, SFC Peak 1) (360)

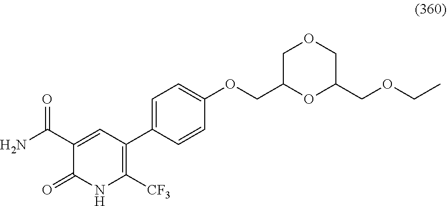 [SMILES: CCOCC1COCC(COc2ccc(-c3cc(C(N)=O)c(=O)[nH]c3C(F)(F)F)cc2)O1]

5-(4-((6-(ethoxymethyl)-1,4-dioxan-2-yl)methoxy)phenyl)-2-oxo-6-(trifluoromethyl)-1,2-dihydropyridine-3-carboxamide (cis, SFC peak 1) (360) was obtained using the procedure described for Example 348, except 2-((4-bromophenoxy)methyl)-6-(difluoromethyl)-1,4-dioxane was replaced with 2-((4-bromophenoxy)methyl)-6-(ethoxymethyl)-1,4-dioxane. ¹H NMR: (DMSO, 400 MHz), δ: 8.63 (br s, 1H), 8.23 (s, 1H), 8.06 (br s, 1H), 7.26 (d, J=8.8 Hz, 2H), 7.03 (d, J=8.8 Hz, 2H), 4.01-3.73 (m, 6H), 3.45-3.41 (m, 2H), 3.38 (m, 2H), 3.33-3.24 (m, 2H), 1.23-0.84 (m, 3H); LCMS: Rt=0.716 min, m/z 457.3 (M+H), Chiral HPLC: Rt=3.23 min, ee value=97.3%.

### 2-((4-bromophenoxy)methyl)-6-(ethoxymethyl)-1,4-dioxane was Obtained Using the Following Procedure

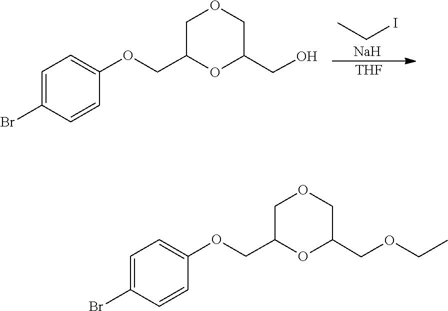

To a solution of (6-((4-bromophenoxy)methyl)-1,4-dioxan-2-yl)methanol (150 mg, 0.50 mmol) in THF (2 mL) was added NaH (30 mg, 075 mmol) at 0° C. The mixture was stirred at 0° C. for 0.5 hour. Iodoethane (390 mg, 2.50 mmol) was added to the mixture at 0° C. and the resulting mixture stirred at 25° C. for 15.5 hrs. The mixture was washed with water (5 mL) and extracted with EA (5 mL×2). The organic layers were combined, dried over anhydrous Na₂SO₄, filtered and concentrated. The residue was purified by column chromatography (PE-PE/EA=5/1) to give 2-((4-bromophenoxy)methyl)-6-(ethoxymethyl)-1,4-dioxane. LCMS: Rt=0.87 min, m/z=331.3, 333.1 (M+H).

### Example 361: 5-(4-((6-(ethoxymethyl)-1,4-dioxan-2-yl)methoxy)phenyl)-2-oxo-6-(trifluoromethyl)-1,2-dihydropyridine-3-carboxamide (Cis, SFC Peak 2) (361)

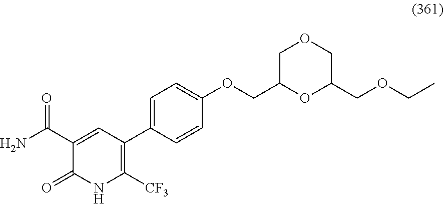 [SMILES: CCOCC1COCC(COc2ccc(-c3cc(C(N)=O)c(=O)[nH]c3C(F)(F)F)cc2)O1]

5-(4-((6-(ethoxymethyl)-1,4-dioxan-2-yl)methoxy)phenyl)-2-oxo-6-(trifluoromethyl)-1,2-dihydropyridine-3-carboxamide (cis, SFC peak 2) (361) was obtained by SFC purification of the product made in Example 359. ¹H NMR: (400 MHz, DMSO-d6), δ: 8.88 (br s, 1H), 8.18 (br s, 1H), 7.97 (br s, 1H), 7.24 (d, J=8.4 Hz, 2H), 7.02 (d, J=8.4 Hz, 2H), 3.99-3.76 (m, 3H), 3.43-3.40 (m, 3H), 3.96-3.32 (m, 4H), 3.21-2.32 (m, 2H), 1.14-1.05 (m, 3H). LCMS: Rt=0.72 min, m/z=457.3 (M+H). Chiral HPLC: Rt=3.46 min, ee value=100%.

### Example 362: 5-(4-((6-((cyclopropylmethoxy)methyl)-1,4-dioxan-2-yl)methoxy)phenyl)-2-oxo-6-(trifluoromethyl)-1,2-dihydropyridine-3-carboxamide (Cis, SFC Peak 1) (362)

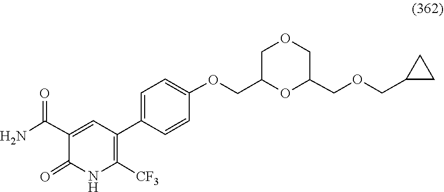 [SMILES: NC(=O)c1cc(-c2ccc(OCC3COCC(COCC4CC4)O3)cc2)c(C(F)(F)F)[nH]c1=O]

5-(4-((6-((cyclopropylmethoxy)methyl)-1,4-dioxan-2-yl)methoxy)phenyl)-2-oxo-6-(trifluoromethyl)-1,2-dihydropyridine-3-carboxamide (cis, SFC peak 1) (362) was obtained using the procedure described for Example 348, except 2-((4-bromophenoxy)methyl)-6-(difluoromethyl)-1,4-dioxane was replaced with 2-((4-bromophenoxy)methyl)-6-((cyclopropylmethoxy)methyl)-1,4-dioxane. ¹H NMR: (400 MHz, DMSO-d6), δ: 8.47 (brs, 1H), 8.08 (s, 1H), 7.91 (br s, 1H), 7.11 (d, J=8.4 Hz, 2H), 6.88 (d, J=8.4 Hz, 2H), 3.86-3.27 (m, 6H), 3.18-3.06 (m, 4H), 1.07 (s, 1H), 0.82-0.71 (m, 1H), 0.32-0.29 (m, 2H), 0.02-−0.012 (m, 2H). LCMS: Rt=0.74 min, m/z=483.3 (M+H). Chiral HPLC: Rt=3.92 min, ee value=100%.

2-((4-bromophenoxy)methyl)-6-((cyclopropylmethoxy)methyl)-1,4-dioxane was obtained using the procedure in the synthesis of 2-((4-bromophenoxy)methyl)-6-(ethoxymethyl)-1,4-dioxane, except iodoethane was replaced with (bromomethyl)cyclopropane.

### Example 363: 5-(4-((6-((cyclopropylmethoxy)methyl)-1,4-dioxan-2-yl)methoxy)phenyl)-2-oxo-6-(trifluoromethyl)-1,2-dihydropyridine-3-carboxamide (Cis, SFC Peak 2) (363)

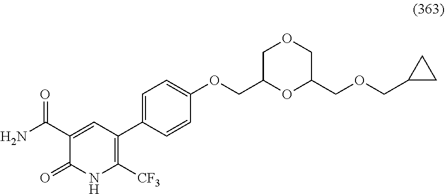 [SMILES: NC(=O)c1cc(-c2ccc(OCC3COCC(COCC4CC4)O3)cc2)c(C(F)(F)F)[nH]c1=O]

5-(4-((6-((cyclopropylmethoxy)methyl)-1,4-dioxan-2-yl)methoxy)phenyl)-2-oxo-6-(trifluoromethyl)-1,2-dihydropyridine-3-carboxamide (cis, SFC peak 2) (363) was obtained by SFC purification of the product made in Example 361. ¹H NMR: (400 MHz, DMSO-d6), δ: 8.54 (br s, 1H), 8.06 (s, 1H), 7.87 (br s, 1H), 7.10 (d, J=8.4 Hz, 2H), 6.88 (d, J=8.8 Hz, 2H), 3.86-3.22 (m, 6H), 3.19-3.06 (m, 4H), 1.19 (br s, 1H), 0.84-0.82 (m, 1H), 0.30-0.28 (m, 2H), 0.013-0.012 (m, 2H). LCMS: Rt=0.75 min, m/z=483.4 (M+H). Chiral HPLC: Rt=4.36 min, ee value=89.6%.

### Example 364: 5-(4-((6-(((1-fluorocyclopropyl)methoxy)methyl)-1,4-dioxan-2-yl)methoxy)phenyl)-2-oxo-6-(trifluoromethyl)-1,2-dihydropyridine-3-carboxamide (Cis, SFC Peak 1) (364)

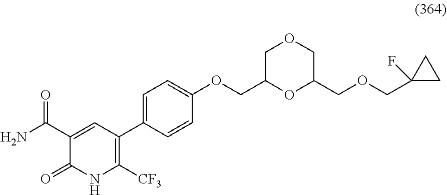 [SMILES: NC(=O)c1cc(-c2ccc(OCC3COCC(COCC4(F)CC4)O3)cc2)c(C(F)(F)F)[nH]c1=O]

5-(4-((6-(((1-fluorocyclopropyl)methoxy)methyl)-1,4-dioxan-2-yl)methoxy)phenyl)-2-oxo-6-(trifluoromethyl)-1,2-dihydropyridine-3-carboxamide (cis, SFC peak 1) (364) was obtained using the procedure described for Example 348, except 2-((4-bromophenoxy)methyl)-6-(difluoromethyl)-1,4-dioxane was replaced with 2-((4-bromophenoxy)methyl)-6-(((1-fluorocyclopropyl)methoxy)methyl)-1,4-dioxane. ¹H NMR: (400 MHz, DMSO-d6), δ: 9.29 (d, J=6.2 Hz, 1H), 8.17 (s, 1H), 7.85 (s, 1H), 7.27 (d, J=8.4 Hz, 2H), 7.06 (d, J=8.6 Hz, 2H), 4.10-3.99 (m, 3H), 3.95-3.82 (m, 3H), 3.80-3.71 (m, 2H), 3.64-3.51 (m, 2H), 3.39-3.33 (m, 2H), 1.13-0.99 (m, 2H), 0.77 (m, 2H). LCMS: Rt=0.77 min, m/z=501.1 (M+H). Chiral HPLC: Rt=3.595 min, ee value=100%.

2-((4-bromophenoxy)methyl)-6-(((1-fluorocyclopropyl)methoxy)methyl)-1,4-dioxane was obtained using the procedure in the synthesis of 2-((4-bromophenoxy)methyl)-6-(ethoxymethyl)-1,4-dioxane, except iodoethane was replaced with 1-(bromomethyl)-1-fluorocyclopropane.

### Example 365: 5-(4-((6-(((1-fluorocyclopropyl)methoxy)methyl)-1,4-dioxan-2-yl)methoxy)phenyl)-2-oxo-6-(trifluoromethyl)-1,2-dihydropyridine-3-carboxamide (Cis, SFC Peak 2) (365)

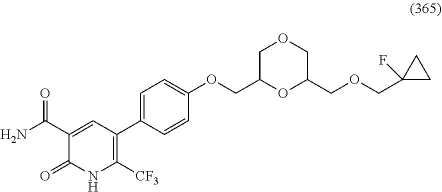 [SMILES: NC(=O)c1cc(-c2ccc(OCC3COCC(COCC4(F)CC4)O3)cc2)c(C(F)(F)F)[nH]c1=O]

5-(4-((6-(((1-fluorocyclopropyl)methoxy)methyl)-1,4-dioxan-2-yl)methoxy)phenyl)-2-oxo-6-(trifluoromethyl)-1,2-dihydropyridine-3-carboxamide (cis, SFC peak 2) (365) was obtained by SFC purification of the product made in Example 361. ¹H NMR: (400 MHz, DMSO-d6), δ: 8.49 (s, 1H), 8.07 (s, 1H), 7.91 (s, 1H), 7.10 (d, J=8.6 Hz, 2H), 6.87 (d, J=8.6 Hz, 2H), 3.93-3.77 (m, 3H), 3.73-3.60 (m, 3H), 3.59-3.50 (m, 2H), 3.35 (m, 2H), 3.13 (d, J=11.0 Hz, 2H), 0.90-0.77 (m, 2H), 0.63-0.49 (m, 2H). LCMS: Rt=0.77 min, m/z=501.1 (M+H). Chiral HPLC: Rt=4.09 min, ee value=92%.

### Example 366: 5-(4-((6-((3,3-difluorocyclobutoxy)methyl)-1,4-dioxan-2-yl)methoxy)phenyl)-2-oxo-6-(trifluoromethyl)-1,2-dihydropyridine-3-carboxamide (Cis, SFC Peak 1) (366)

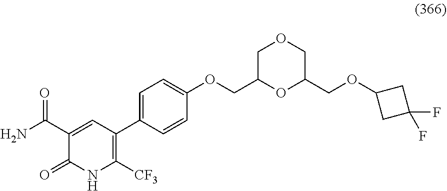 [SMILES: NC(=O)c1cc(-c2ccc(OCC3COCC(COC4CC(F)(F)C4)O3)cc2)c(C(F)(F)F)[nH]c1=O]

5-(4-((6-((3,3-difluorocyclobutoxy)methyl)-1,4-dioxan-2-yl)methoxy)phenyl)-2-oxo-6-(trifluoromethyl)-1,2-dihydropyridine-3-carboxamide (cis, SFC peak 1) (366) was obtained using the procedure described for Example 348, except 2-((4-bromophenoxy)methyl)-6-(difluoromethyl)-1,4-dioxane was replaced with 2-((4-bromophenoxy)methyl)-6-((3,3-difluorocyclobutoxy)methyl)-1,4-dioxane. ¹H NMR: (400 MHz, DMSO-d6), δ=8.63 (s, 1H), 8.23 (s, 1H), 8.08 (s, 1H), 7.25 (d, J=8.4 Hz, 2H), 7.02 (d, J=8.8 Hz, 2H), 4.09-3.92 (m, 4H), 3.89-3.71 (m, 3H), 3.29-3.18 (m, 2H), 2.93-2.78 (m, 2H). LCMS: Rt=0.79 min, m/z=519.0 (M+H). Chiral HPLC: Rt=1.74 min, ee value=94%, Amycoat-MeOH(DEA)-30-7 min-3 mL.

2-((4-bromophenoxy)methyl)-6-((3,3-difluorocyclobutoxy)methyl)-1,4-dioxane was obtained using the procedure in the synthesis of 2-((4-bromophenoxy)methyl)-6-(ethoxymethyl)-1,4-dioxane, except iodoethane was replaced with 3-bromo-1,1-difluorocyclobutane.

### Example 367: 5-(4-((6-((3,3-difluorocyclobutoxy)methyl)-1,4-dioxan-2-yl)methoxy)phenyl)-2-oxo-6-(trifluoromethyl)-1,2-dihydropyridine-3-carboxamide (Cis, SFC Peak 2) (367)

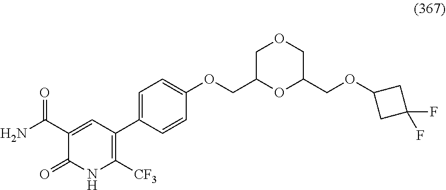 [SMILES: NC(=O)c1cc(-c2ccc(OCC3COCC(COC4CC(F)(F)C4)O3)cc2)c(C(F)(F)F)[nH]c1=O]

5-(4-((6-((3,3-difluorocyclobutoxy)methyl)-1,4-dioxan-2-yl)methoxy)phenyl)-2-oxo-6-(trifluoromethyl)-1,2-dihydropyridine-3-carboxamide (cis, SFC peak 2) (367) was obtained by SFC purification of the product made in Example 366. ¹H NMR: (400 MHz, DMSO-d6), δ=8.66 (s, 1H), 8.22 (s, 1H), 8.06 (s, 1H), 7.25 (d, J=8.0 Hz, 2H), 7.02 (d, J=8.4 Hz, 2H), 4.00 (m, 5H), 3.89-3.70 (m, 4H), 3.30-3.20 (m, 4H), 2.97-2.77 (m, 2H). LCMS: Rt=0.79 min, m/z=519.0 (M+H). Chiral HPLC: Rt=2.22 min, ee value=95%, Amycoat-MeOH(DEA)-30-7 min-3 mL.

### Example 368: 5-(4-((6-((2,2-difluoroethoxy)methyl)-1,4-dioxan-2-yl)methoxy)phenyl)-2-oxo-6-(trifluoromethyl)-1,2-dihydropyridine-3-carboxamide (Cis, SFC Peak 1) (368)

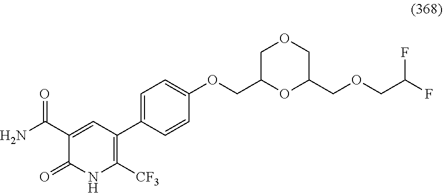 [SMILES: NC(=O)c1cc(-c2ccc(OCC3COCC(COCC(F)F)O3)cc2)c(C(F)(F)F)[nH]c1=O]

5-(4-((6-((2,2-difluoroethoxy)methyl)-1,4-dioxan-2-yl)methoxy)phenyl)-2-oxo-6-(trifluoromethyl)-1,2-dihydropyridine-3-carboxamide (cis, SFC peak 1) (368) was obtained using the procedure described for Example 348, except 2-((4-bromophenoxy)methyl)-6-(difluoromethyl)-1,4-dioxane was replaced with 2-((4-bromophenoxy)methyl)-6-((2,2-difluoroethoxy)methyl)-1,4-dioxane. ¹H NMR (400 MHz, MeOD-d4) δ=8.27 (s, 1H), 7.25 (d, J=8.8 Hz, 2H), 7.01 (d, J=8.8 Hz, 2H), 6.09-5.79 (m, 1H), 4.08-4.02 (m, 3H), 3.90-3.71 (m, 3H), 3.63-3.60 (m, 4H), 3.59-3.31 (m, 2H). 19F NMR (377 MHz, MeOD-d4) δ=−62.77 (s), −122.36 (s). LCMS: Rt=0.875 min, m/z=493.3 (M+H). Chiral HPLC: Rt=0.72 min, de value=99%, AD-3-MeOH (DEA)-40-7 min-3 mL-35.

2-((4-bromophenoxy)methyl)-6-((2,2-difluoroethoxy)methyl)-1,4-dioxane was obtained using the procedure in the synthesis of 2-((4-bromophenoxy)methyl)-6-(ethoxymethyl)-1,4-dioxane, except iodoethane was replaced with 1,1-difluoro-2-iodoethane.

### Example 369: 5-(4-((6-((2,2-difluoroethoxy)methyl)-1,4-dioxan-2-yl)methoxy)phenyl)-2-oxo-6-(trifluoromethyl)-1,2-dihydropyridine-3-carboxamide (Cis, SFC Peak 2) (369)

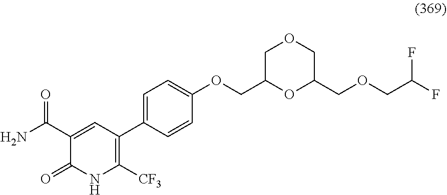 [SMILES: NC(=O)c1cc(-c2ccc(OCC3COCC(COCC(F)F)O3)cc2)c(C(F)(F)F)[nH]c1=O]

5-(4-((6-((2,2-difluoroethoxy)methyl)-1,4-dioxan-2-yl)methoxy)phenyl)-2-oxo-6-(trifluoromethyl)-1,2-dihydropyridine-3-carboxamide (cis, SFC peak 2) (369) was obtained by SFC purification of the product made in Example 368. ¹H NMR (400 MHz, MeOD-d4) δ=8.25 (s, 1H), 7.24 (d, J=8.8 Hz, 2H), 7.00 (d, J=8.8 Hz, 2H), 6.09-5.79 (m, 1H), 4.05-4.02 (m, 3H), 3.90-3.71 (m, 3H), 3.63-3.60 (m, 4H), 3.59-3.39 (m, 2H). 19F NMR (377 MHz, MeOD-d4) δ=−62.77 (s, 3F), −122.37 (s). LCMS: Rt=0.88 min, m/z=492.9 (M+H). Chiral HPLC: Rt=0.995 min, de value=95%, AD-3-MeOH(DEA)-40-7 min-3 mL-3.

### Example 370: 5-(4-((6-((2-methoxy-2-methylpropoxy)methyl)-1,4-dioxan-2-yl)methoxy)phenyl)-2-oxo-6-(trifluoromethyl)-1,2-dihydropyridine-3-carboxamide (Cis, SFC Peak 1) (370)

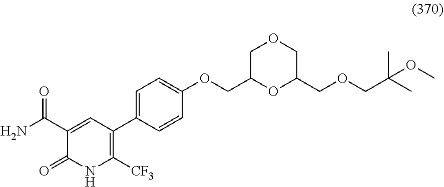 [SMILES: COC(C)(C)COCC1COCC(COc2ccc(-c3cc(C(N)=O)c(=O)[nH]c3C(F)(F)F)cc2)O1]

5-(4-((6-((2-methoxy-2-methylpropoxy)methyl)-1,4-dioxan-2-yl)methoxy)phenyl)-2-oxo-6-(trifluoromethyl)-1,2-dihydropyridine-3-carboxamide (cis, SFC peak 1) (370) was obtained using the procedure described for Example 348, except 2-((4-bromophenoxy)methyl)-6-(difluoromethyl)-1,4-dioxane was replaced with 2-((4-bromophenoxy)methyl)-6-((2-methoxy-2-methylpropoxy)methyl)-1,4-dioxane. ¹H NMR: (400 MHz, DMSO-d6), δ: 9.23 (s, 1H), 8.17 (s, 1H), 7.85 (s, 1H), 7.27 (d, J=8.0 Hz, 2H), 7.05 (d, J=8.4 Hz, 2H), 4.11-3.98 (m, 3H), 3.95-3.81 (m, 3H), 3.38-3.30 (m, 6H), 3.16 (s, 3H), 1.12 (s, 6H). LCMS: Rt=0.70 min, m/z=515.3 (M+H). Chiral HPLC: Rt=3.59 min, ee value=98%.

2-((4-bromophenoxy)methyl)-6-((2-methoxy-2-methylpropoxy)methyl)-1,4-dioxane was obtained using the procedure in the synthesis of 2-((4-bromophenoxy)methyl)-6-(ethoxymethyl)-1,4-dioxane, except iodoethane was replaced with 1-iodo-2-methoxy-2-methylpropane.

### Example 371: 5-(4-((6-((2-methoxy-2-methylpropoxy)methyl)-1,4-dioxan-2-yl)methoxy)phenyl)-2-oxo-6-(trifluoromethyl)-1,2-dihydropyridine-3-carboxamide (Cis, SFC Peak 2) (371)

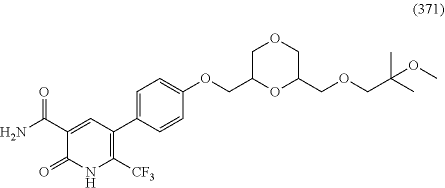 [SMILES: COC(C)(C)COCC1COCC(COc2ccc(-c3cc(C(N)=O)c(=O)[nH]c3C(F)(F)F)cc2)O1]

5-(4-((6-((2-methoxy-2-methylpropoxy)methyl)-1,4-dioxan-2-yl)methoxy)phenyl)-2-oxo-6-(trifluoromethyl)-1,2-dihydropyridine-3-carboxamide (cis, SFC peak 2) (371) was obtained by SFC purification of the product made in Example 370. ¹H NMR: (400 MHz, DMSO-d6), δ: 10.11 (s, 1H), 8.00 (s, 1H), 7.42 (s, 1H), 7.21 (d, J=8.4 Hz, 2H), 7.01 (br d, J=8.6 Hz, 2H), 4.12-3.97 (m, 3H), 3.94-3.80 (m, 3H), 3.29 (s, 6H), 3.16 (s, 3H), 1.12 (s, 6H). LCMS: Rt=0.72 min, m/z=515.3 (M+H). Chiral HPLC: Rt=4.09 min, ee value=95.5%.

### Example 372: 2-oxo-5-(4-((6-((trifluoromethoxy)methyl)-1,4-dioxan-2-yl)methoxy)phenyl)-6-(trifluoromethyl)-1,2-dihydropyridine-3-carboxamide (Cis, SFC Peak 1) (372)

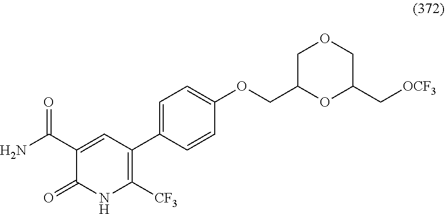 [SMILES: NC(=O)c1cc(-c2ccc(OCC3COCC(COC(F)(F)F)O3)cc2)c(C(F)(F)F)[nH]c1=O]

2-oxo-5-(4-((6-((trifluoromethoxy)methyl)-1,4-dioxan-2-yl)methoxy)phenyl)-6-(trifluoromethyl)-1,2-dihydropyridine-3-carboxamide (cis, SFC peak 1) (372) was obtained using the procedure described for Example 348, except 2-((4-bromophenoxy)methyl)-6-(difluoromethyl)-1,4-dioxane was replaced with 2-((4-bromophenoxy)methyl)-6-((trifluoromethoxy)methyl)-1,4-dioxane. ¹H NMR (400 MHz, DMSO-d6) δ=9.05 (s, 1H), 8.14 (s, 1H), 8.01-7.70 (m, 1H), 7.23 (d, J=8.4 Hz, 2H), 7.02 (d, J=8.6 Hz, 2H), 4.18-3.79 (m, 9H), 3.30 (s, 1H). LCMS: Rt=0.83 min, m/z=497.0 (M+H). Chiral HPLC: Rt=2.65 min, ee value=100%.

### 2-((4-bromophenoxy)methyl)-6-((trifluoromethoxy)methyl)-1,4-dioxane was Obtained Using the Following Procedure

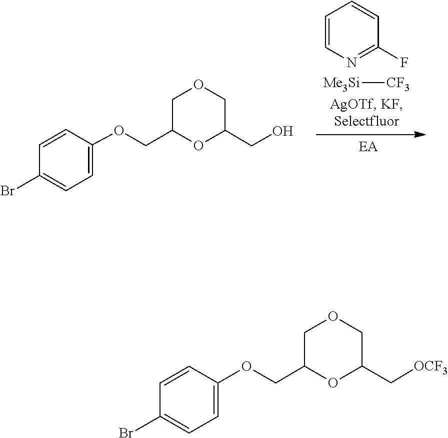

To a solution of (6-((4-bromophenoxy)methyl)-1,4-dioxan-2-yl)methanol (600 mg, 2 mmol), AgOTf (1 g, 4.0 mmol), Selectfluor (1 g, 3 mmol), KF (350 mg, 6 mmol) in EA (8 mL) was added 2-fluorpyridine (400 mg, 4 mmol) and trimethyl(trifluoromethyl)silane (570 mg, 4 mmol). The mixture was stirred at 25° C. for 16 hrs. The reaction mixture was poured into water (10 mL) and the resulting mixture extracted with EA (10 mL×2). The organic layers were combined, dried over anhydrous Na₂SO₄, filtered and concentrated to give crude product which was purified by Prep-TLC (PE:EA=1:1) to give 2-((4-bromophenoxy)methyl)-6-((trifluoromethoxy)methyl)-1,4-dioxane. ¹H NMR (400 MHz, CDCl6) δ=7.47-7.34 (m, 2H), 6.87-6.76 (m, 2H), 4.05-3.88 (m, 8H), 3.49-3.39 (m, 2H).

### Example 373: 2-oxo-5-(4-((6-((trifluoromethoxy)methyl)-1,4-dioxan-2-yl)methoxy)phenyl)-6-(trifluoromethyl)-1,2-dihydropyridine-3-carboxamide (Cis, SFC Peak 2) (373)

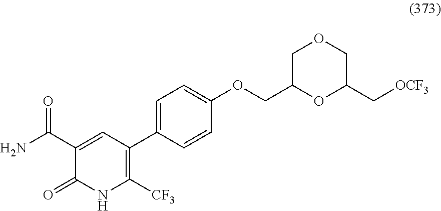 [SMILES: NC(=O)c1cc(-c2ccc(OCC3COCC(COC(F)(F)F)O3)cc2)c(C(F)(F)F)[nH]c1=O]

2-oxo-5-(4-((6-((trifluoromethoxy)methyl)-1,4-dioxan-2-yl)methoxy)phenyl)-6-(trifluoromethyl)-1,2-dihydropyridine-3-carboxamide (cis, SFC peak 2) (373) was obtained by SFC purification of the product made in Example 372. ¹H NMR (400 MHz, DMSO-d6) δ=9.73 (s, 1H), 8.01 (s, 1H), 7.63-7.44 (m, 1H), 7.18 (d, J=8.4 Hz, 2H), 6.99 (d, J=8.6 Hz, 2H), 4.35-3.74 (m, 9H), 3.30-3.28 (m, 1H). LCMS: Rt=0.83 min, m/z=497.0 (M+H). Chiral HPLC: Rt=2.90 min, ee value=86.12%.

### Example 374: 5-(4-((6-((1H-pyrrol-1-ylmethyl)-1,4-dioxan-2-yl)methoxy)phenyl)-2-oxo-6-(trifluoromethyl)-1,2-dihydropyridine-3-carboxamide (Cis, SFC Peak 1) (374)

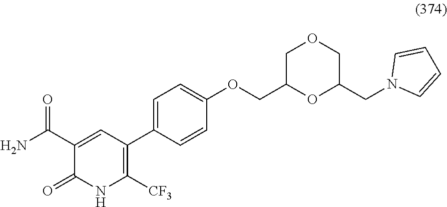 [SMILES: NC(=O)c1cc(-c2ccc(OCC3COCC(Cn4cccc4)O3)cc2)c(C(F)(F)F)[nH]c1=O]

5-(4-((6-((1H-pyrrol-1-yl)methyl)-1,4-dioxan-2-yl)methoxy)phenyl)-2-oxo-6-(trifluoromethyl)-1,2-dihydropyridine-3-carboxamide (cis, SFC peak 1) (374) was obtained using the procedure described for Example 348, except 2-((4-bromophenoxy)methyl)-6-(difluoromethyl)-1,4-dioxane was replaced with 1-((6-((4-bromophenoxy)methyl)-1,4-dioxan-2-yl)methyl)-1H-pyrrole. ¹H NMR (400 MHz, DMSO-d6) δ=8.12 (s, 1H), 7.80 (s, 1H), 7.22 (d, J=8.0 Hz, 2H), 7.00 (d, J=8.0 Hz, 2H), 6.74 (s, 2H), 5.98 (s, 2H), 4.01 (s, 2H), 3.95-3.94 (m, 4H), 3.86-3.83 (m, 2H), 3.60-3.58 (m, 1H), 3.15-3.09 (m, 1H). LCMS: Rt=0.67 min, m/z=478.1 (M+H). Chiral HPLC: Rt=2.03 min, ee value=100%.

### 1-((6-((4-bromophenoxy)methyl)-1,4-dioxan-2-yl)methyl)-1H-pyrrole was Obtained Using the Following Procedure

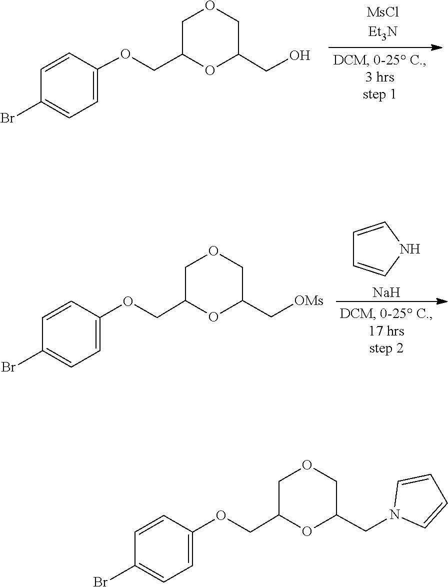

Step 1: To a solution of (6-((4-bromophenoxy)methyl)-1,4-dioxan-2-yl)methanol (1.0 g, 3.30 mmol) in anhydrous DCM (20 mL) was added Et₃N (668 mg, 6.60 mmol) and MsCl (567 mg, 4.95 mmol) at 0° C. and the mixture stirred at 25° C. for 3 hrs. The reaction mixture was poured into water (20 mL) and the organic phase was separated and dried over anhydrous Na2SO4, filtered and concentrated to give crude product. The crude product was purified by silica gel column (PE) to give (6-((4-bromophenoxy)methyl)-1,4-dioxan-2-yl)methyl methanesulfonate. ¹H NMR (400 MHz, CDCl3) δ=7.45-7.35 (m, 2H), 6.82-6.71 (m, 2H), 4.25-4.24 (m 2H), 4.10-3.82 (m, 6H), 3.50-3.39 (m, 2H), 3.07 (s, 3H). LCMS: Rt=0.82 min, m/z=381/383 (M+H).

Step 2: To a solution of pyrrole (30 mg, 0.44 mmol) in anhydrous DMF (2 mL), then NaH (21 mg, 0.52 mool) was added at 0° C., The reaction was stirred at 0° C. for 0.5 hrs and (6-((4-bromophenoxy)methyl)-1,4-dioxan-2-yl)methyl methanesulfonate (200 mg, 0.52 mmol) added. The reaction was stirred at 25° C. for 17 hrs. The reaction mixture was poured into H₂O (10 mL), and extracted with EA (10 mL×3). The organic layers were combined, washed with brine (20 mL), dried over anhydrous Na₂SO₄, filtered and concentrated to give crude product which was purified by reversed phase column chromatography (basic) to give 1-((6-((4-bromophenoxy)methyl)-1,4-dioxan-2-yl)methyl)-1H-pyrrole. LCMS: Rt=1.02 min, m/z=352.0/354.0 (M+H).

### Example 375: 5-(4-((6-((1H-pyrrol-1-yl)methyl)-1,4-dioxan-2-yl)methoxy)phenyl)-2-oxo-6-(trifluoromethyl)-1,2-dihydropyridine-3-carboxamide (Cis, SFC Peak 2) (375)

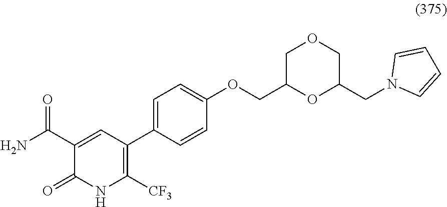 [SMILES: NC(=O)c1cc(-c2ccc(OCC3COCC(Cn4cccc4)O3)cc2)c(C(F)(F)F)[nH]c1=O]

5-(4-((6-((1H-pyrrol-1-yl)methyl)-1,4-dioxan-2-yl)methoxy)phenyl)-2-oxo-6-(trifluoromethyl)-1,2-dihydropyridine-3-carboxamide (cis, SFC peak 2) (375) was obtained by SFC purification of the product made in Example 374. ¹H NMR (400 MHz, DMSO-d6) δ=8.09 (s, 1H), 7.21 (d, J=8.0 Hz, 2H), 6.99 (d, J=8.0 Hz, 2H), 6.74 (s, 2H), 5.98 (s, 2H), 4.01-4.00 (m, 3H), 3.95-3.94 (m, 3H), 3.86-3.83 (m, 2H), 3.60-3.58 (m, 1H), 3.17-3.08 (m, 1H). LCMS: Rt=0.67 min, m/z=478.1 (M+H). Chiral HPLC: Rt=2.91 min, ee value=95%.

### Example 376: 5-(4-((6-((1H-pyrazol-1-ylmethyl)-1,4-dioxan-2-yl)methoxy)phenyl)-2-oxo-6-(trifluoromethyl)-1,2-dihydropyridine-3-carboxamide (Cis, SFC Peak 1) (376)

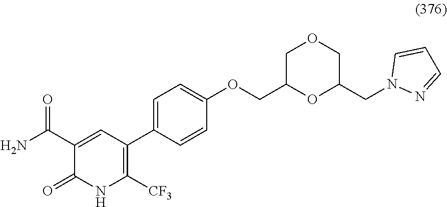 [SMILES: NC(=O)c1cc(-c2ccc(OCC3COCC(Cn4cccn4)O3)cc2)c(C(F)(F)F)[nH]c1=O]

5-(4-((6-((1H-pyrazol-1-yl)methyl)-1,4-dioxan-2-yl)methoxy)phenyl)-2-oxo-6-(trifluoromethyl)-1,2-dihydropyridine-3-carboxamide (cis, SFC peak 1) (376) was obtained using the procedure described for Example 348, except 2-((4-bromophenoxy)methyl)-6-(difluoromethyl)-1,4-dioxane was replaced with 1-((6-((4-bromophenoxy)methyl)-1,4-dioxan-2-yl)methyl)-1H-pyrazole. ¹H NMR (400 MHz, DMSO-d6) δ=8.50 (s, 1H), 8.26 (s, 1H), 8.14 (s, 1H), 7.69-7.68 (m, 1H), 7.45 (s, 1H), 7.26 (d, J=8.0 Hz, 2H), 7.02 (d, J=8.0 Hz, 2H), 6.24 (s, 1H), 4.19-4.18 (m, 2H), 4.03-3.95 (m, 4H), 3.86-3.83 (m, 2H), 3.70-3.64 (m, 1H), 3.21-3.15 (m, 1H). LCMS: Rt=0.62 min, m/z=479.1 (M+H). Chiral HPLC: Rt=1.39 min, ee value=86%.

### Example 377: 5-(4-((6-((1H-pyrazol-1-ylmethyl)-1,4-dioxan-2-yl)methoxy)phenyl)-2-oxo-6-(trifluoromethyl)-1,2-dihydropyridine-3-carboxamide (Cis, SFC Peak 2) (377)

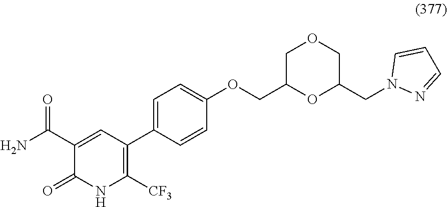 [SMILES: NC(=O)c1cc(-c2ccc(OCC3COCC(Cn4cccn4)O3)cc2)c(C(F)(F)F)[nH]c1=O]

5-(4-((6-((1H-pyrazol-1-yl)methyl)-1,4-dioxan-2-yl)methoxy)phenyl)-2-oxo-6-(trifluoromethyl)-1,2-dihydropyridine-3-carboxamide (cis, SFC peak 2) was obtained by SFC purification of the product made in Example 376. ¹H NMR (400 MHz, DMSO-d6) 9.73-9.26 (m, 1H), 8.06 (s, 1H), 7.82-7.61 (m, 2H), 7.45 (s, 1H), 7.19 (d, J=8.0 Hz, 2H), 6.98 (d, J=8.0 Hz, 2H), 6.24 (s, 1H), 4.20-4.19 (m, 2H), 4.15-3.94 (m, 4H), 3.86-3.84 (m, 2H), 3.69-3.66 (m, 1H), 3.24-3.15 (m, 1H). LCMS: Rt=0.62 min, m/z=479.1 (M+H). Chiral HPLC: Rt=1.45 min, ee value=75%.

### Example 378: 5-(4-((6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide (Cis, SFC Peak 1) (378)

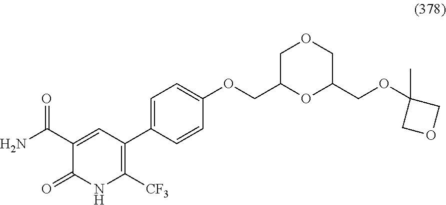 [SMILES: CC1(OCC2COCC(COc3ccc(-c4cc(C(N)=O)c(=O)[nH]c4C(F)(F)F)cc3)O2)COC1]

5-(4-((6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide (cis, SFC peak 1) (378) was obtained using the procedure described for Example 348, except 2-((4-bromophenoxy)methyl)-6-(difluoromethyl)-1,4-dioxane was replaced with 2-((4-bromophenoxy)methyl)-6-(((3-methyl)oxetan-3-yl)oxy)methyl)-1,4-dioxane. ¹H NMR: (400 MHz, DMSO-d6), δ=8.84 (brs, 1H), 8.18 (s, 1H), 7.97 (br s, 1H), 7.25 (d, J=8.4 Hz, 2H), 7.03 (d, J=8.4 Hz, 2H), 4.51 (d, J=6.4 Hz, 2H), 4.26 (d, J=6.4 Hz, 2H), 4.02 (m, 6H), 3.42 (m, 2H), 3.34 (m, 2H), 1.40 (s, 3H). LCMS: Rt=0.70 min, m/z=499.4 (M+H). Chiral HPLC: Rt=1.01 min, ee value=98%.

### Example 379: 5-(4-((6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide (Cis, SFC Peak 2) (379)

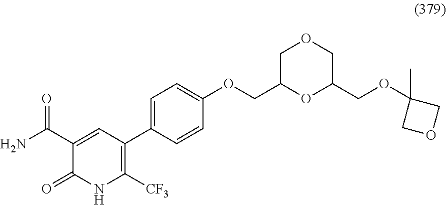 [SMILES: CC1(OCC2COCC(COc3ccc(-c4cc(C(N)=O)c(=O)[nH]c4C(F)(F)F)cc3)O2)COC1]

5-(4-((6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide (cis, SFC peak 2) (379) was obtained by SFC purification of the product made in Example 378. ¹H NMR: (400 MHz, DMSO-d6), δ=9.01 (br s, 1H), 8.15 (s, 1H), 7.89 (br s, 1H), 7.24 (d, J=8.4 Hz, 2H), 7.02 (d, J=8.4 Hz, 2H), 4.51 (d, J=6.4 Hz, 2H), 4.26 (d, J=6.4 Hz, 2H), 4.00 (m, 6H), 3.41 (m, 2H), 3.30 (m, 2H), 1.43 (s, 3H). LCMS: Rt=0.70 min, m/z=499.3 (M+H). Chiral HPLC: Rt=1.36 min, ee value=93%.

### Example 380: 5-(4-((3-cyclopropyl-6,7-dihydro-4H-[1,2,3]triazolo[5,1-c][1,4]oxazin-6-yl)methoxy)phenyl)-2-oxo-6-(trifluoromethyl)-1,2-dihydropyridine-3-carboxamide (380)

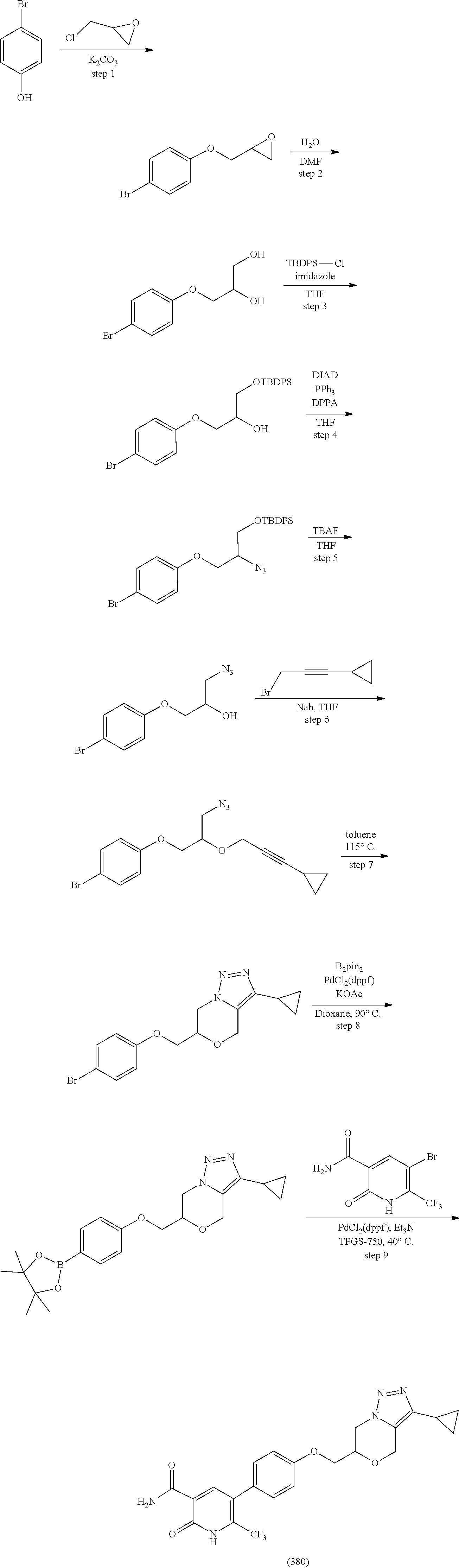

Step 1: To an oven-dried 500 mL flask was added 4-bromophenol (10.0 g, 57.8 mmol) and potassium carbonate (39.9 g, 289 mmol). The vessel was sealed and purged with vac/N₂ (x)₃ and acetonitrile (100 mL) and epichlorohydrin (22.60 mL, 289 mmol) were added. A reflux condenser was attached and the reaction heated to 100° C. for 3 h. The reaction mixture was cooled to rt, diluted with Et₂O (1.5 L), washed with 2M NaOH, 1:1 brine/water (×2), dried over Na₂SO₄, filtered through SiO₂ and concentrated in vacuo to give 2-((4-bromophenoxy)methyl)oxirane.

Step 2: To a 5 mL microwave vial was added 2-((4-bromophenoxy)methyl)oxirane (1.5 g, 6.55 mmol). The solids were melted via heat gun and water (0.354 ml, 19.64 mmol) and DMF (0.076 ml, 0.98 mmol) were added. The vial was sealed and heated to 110° C. for 22 hours. The reaction was diluted with EtOAc and washed with brine, then dried over sodium sulfate and concentrated. The crude material was purified via silica gel chromatography (ISCO, 0 to 70% ethyl acetate/heptane) to give 3-(4-bromophenoxy)propane-1,2-diol. LCMS Rt=0.69 min, m/z=246.9, 248.8 (M+H).

Step 3: To a 100 mL flask was 3-(4-bromophenoxy)propane-1,2-diol (700 mg, 2.83 mmol) and imidazole (289 mg, 4.25 mmol). Dry THF (14 mL) was added, followed by dropwise addition of TBDPS-Cl (0.958 ml, 3.68 mmol). The reaction was then stirred at RT for 3 hours, quenched with MeOH (0.5 mL), filtered, and concentrated down. The crude material was purified via silica gel chromatography (ISCO, 0 to 20% ethyl acetate/heptane) to give 1-(4-bromophenoxy)-3-((tert-butyldiphenylsilyl)oxy)propan-2-ol. ¹H NMR (500 MHz, CDCl₃) δ 7.67-7.62 (m, 4H), 7.47-7.41 (m, 2H), 7.40-7.33 (m, 6H), 6.76 (d, J=8.9 Hz, 2H), 4.08 (h, J=5.3 Hz, 1H), 4.04-3.97 (m, 2H), 3.83 (d, J=5.0 Hz, 2H), 2.53 (d, J=5.8 Hz, 1H), 1.07 (s, 9H).

Step 4: A solution of 1-(4-bromophenoxy)-3-((tert-butyidiphenylsilyl)oxy)propan-2-ol (1.18 g, 2.43 mmol) in dry THF (25 mL) was purged with N₂ and cooled to 0° C. Under N₂, triphenylphosphine (1.275 g, 4.86 mmol) was added, followed by DIAD (1.000 mL, 5.08 mmol) and DPPA (0.926 mL, 4.86 mmol). After stirring the mixture at 0° C. for 30 min, the reaction was warmed to RT and stirred for 6 hours. The crude material was filtered, concentrated, and purified via silica gel chromatography twice (ISCO, first with 0 to 10% ethyl acetate/heptane, then with 30% DCM/heptane) to give (2-azido-3-(4-bromophenoxy)propoxy)(tert-butyl)diphenylsilane. The material was used in the next step without further purification.

Step 5: To a 100 mL flask was added (2-azido-3-(4-bromophenoxy)propoxy)(tert-butyl)diphenylsilane (760 mg, 1.49 mmol), dry THF (15 mL), and TBAF (1M solution in THF, 1.9 ml, 1.9 mmol). The reaction was stirred at RT for 30 min. The reaction was quenched with sat. NH₄Cl, extracted with EtOAc twice, dried over sodium sulfate, and concentrated down. The crude material was purified via silica gel chromatography (ISCO, 0 to 30% ethyl acetate/heptane) to give 1-azido-3-(4-bromophenoxy)propan-2-ol. ¹H NMR (500 MHz, CDCl₃) δ 7.41 (d, J=8.3 Hz, 2H), 6.82 (d, J=8.2 Hz, 2H), 4.19 (t, J=5.5 Hz, 1H), 4.00 (d, J=5.3 Hz, 2H), 3.55 (qd, J=12.7, 5.3 Hz, 2H), 2.48 (s, 1H)

Step 6: To a 50 mL flask was added 1-azido-3-(4-bromophenoxy)propan-2-ol (90 mg, 0.333 mmol) and dry THF (3 mL). The mixture was cooled to 0° C., then NaH (26.5 mg, 0.662 mmol) was added and the reaction stirred at 0° C. for 30 min. (3-bromo-1-propyn-1-yl)cyclopropane (0.053 mL, 0.496 mmol) was then added, the ice bath removed and the reaction let warm to RT and stirred for 16 hours. The reaction was quenched with sat. NH₄Cl, extracted with EtOAc twice, dried over sodium sulfate, and concentrated. The crude material was used in the next step without further purification. LCMS Rt=1.13 min, m/z=349.9, 351.9 (M+H).

Step 7: To a microwave vial was added 1-(3-azido-2-((3-cyclopropylprop-2-yn-1-yl)oxy)propoxy)-4-bromobenzene (116 mg, crude) and dry toluene (3 mL). The vial was sealed and heated to 115° C. for 90 min. The crude material was then directly purified via silica gel chromatography (ISCO, 0 to 70% ethyl acetate/heptane) to give 6-((4-bromophenoxy)methyl)-3-cyclopropyl-6,7-dihydro-4H-[1,2,3]triazolo[5,1-c][1,4]oxazine. LCMS Rt=0.92 min, m/z=349.9, 351.9 (M+H).

Step 8: To a 5 mL microwave vial was added bis(pinacolato)diboron (83 mg, 0.327 mmol, PdCl₂(dppf)-DCM complex (20.5 mg, 0.025 mmol), and potassium acetate (74 mg, 0.754 mmol. The vial was purged with N₂ for a few minutes, then under a stream of N₂, a solution of 6-((4-bromophenoxy)methyl)-3-cyclopropyl-6,7-dihydro-4H-[1,2,3]triazolo[5,1-c][1,4]oxazine (88 mg, 0.251 mmol) in dry dioxane (2.5 mL) was added. The vial was sealed and heated to 90° C. for 1 hour. The crude was concentrated down and purified via silica gel chromatography (ISCO, 0 to 70% ethyl acetate/heptane) to give 3-cyclopropyl-6-((4-(4,4,5,5-tetramethyl-1,3,2-dioxaborolan-2-yl)phenoxy)methyl)-6,7-dihydro-4H-[1,2,3]triazolo[5,1-c][1,4]oxazine. LCMS Rt=0.99 min, m/z=398.1 (M+H).

Step 9: To a 50 mL flask was added 3-cyclopropyl-6-((4-(4,4,5,5-tetramethyl-1,3,2-dioxaborolan-2-yl)phenoxy)methyl)-6,7-dihydro-4H-[1,2,3]triazolo[5,1-c][1,4]oxazine (92 mg, 0.232 mmol, 5-bromo-2-oxo-6-(trifluoromethyl)-1,2-dihydropyridine-3-carboxamide (60 mg, 0.211 mmol, and PdCl₂(dtbpf) (5.16 mg, 5.26 μmol). The flask was purged with N₂ for several minutes, then under a stream of N₂, TPGS-750 2 w % (2 mL) was added, followed by triethylamine (0.147 mL, 1.05 mmol. The reaction was then heated to 40° C. for 64 hours. The reaction was acidified with 1M HCl, extracted with EtOAc twice, dried over sodium sulfate, and concentrated down. The residue was dissolved in DMSO and purified via reverse phase HPLC. The pure fractions were lyophilized to give 5-(4-((3-cyclopropyl-6,7-dihydro-4H-[1,2,3]triazolo[5,1-c][1,4]oxazin-6-yl)methoxy)phenyl)-2-oxo-6-(trifluoromethyl)-1,2-dihydropyridine-3-carboxamide. ¹H NMR (500 MHz, DMSO-d6) δ 8.47 (s, 1H), 8.30 (s, 1H), 8.19 (s, 1H), 7.32 (d, J=8.7 Hz, 2H), 7.11 (d, J=8.8 Hz, 2H), 5.16 (d, J=14.9 Hz, 1H), 4.89 (d, J=15.0 Hz, 1H), 4.62 (dd, J=12.7, 3.4 Hz, 1H), 4.37 (ddd, J=13.6, 6.3, 3.3 Hz, 1H), 4.33 (dd, J=10.8, 3.2 Hz, 1H), 4.28 (dd, J=10.8, 6.2 Hz, 1H), 4.20 (dd, J=12.7, 10.6 Hz, 1H), 1.85 (tt, J=8.4, 5.1 Hz, 1H), 0.93-0.85 (m, 2H), 0.82-0.74 (m, 2H). LCMS Rt=3.13 min, m/z=476.0 (M+H).

### Example 381: 5-(4-((3-cyclopropyl-6,7-dihydro-4H-[1,2,3]triazolo[5,1-c][1,4]oxazin-7-yl)methoxy)phenyl)-2-oxo-6-(trifluoromethyl)-1,2-dihydropyridine-3-carboxamide (381)

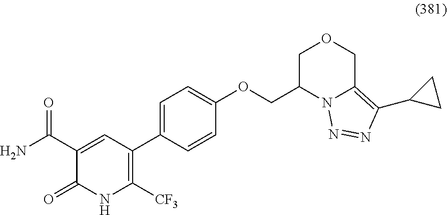 [SMILES: NC(=O)c1cc(-c2ccc(OCC3COCc4c(C5CC5)nnn43)cc2)c(C(F)(F)F)[nH]c1=O]

5-(4-((3-cyclopropyl-6,7-dihydro-4H-[1,2,3]triazolo[5,1-c][1,4]oxazin-7-yl)methoxy)phenyl)-2-oxo-6-(trifluoromethyl)-1,2-dihydropyridine-3-carboxamide (381) was obtained using the procedure described for Example 348, except 2-((4-bromophenoxy)methyl)-6-(difluoromethyl)-1,4-dioxane was replaced with 7-((4-bromophenoxy)methyl)-3-cyclopropyl-6,7-dihydro-4H-[1,2,3]triazolo[5,1-c][1,4]oxazine. ¹H NMR (500 MHz, DMSO-d₆) δ 13.62 (s, 1H), 8.45 (s, 1H), 8.27 (s, 1H), 8.17 (s, 1H), 7.28 (d, J=8.1 Hz, 2H), 7.07 (d, J=8.0 Hz, 2H), 4.98 (d, J=15.0 Hz, 1H), 4.94-4.88 (m, 2H), 4.50 (t, J=8.6 Hz, 1H), 4.43 (dd, J=10.6, 4.1 Hz, 1H), 4.22-4.11 (m, 2H), 1.84 (p, J=6.6 Hz, 1H), 0.87 (d, J=8.5 Hz, 2H), 0.81-0.74 (m, 2H). LCMS Rt=0.75 min, m/z=476.3 (M+H).

### 7-((4-bromophenoxy)methyl)-3-cyclopropyl-6,7-dihydro-4H-[1,2,3]triazolo[5,1-c][1,4]oxazine was Obtained Using the Following Procedure

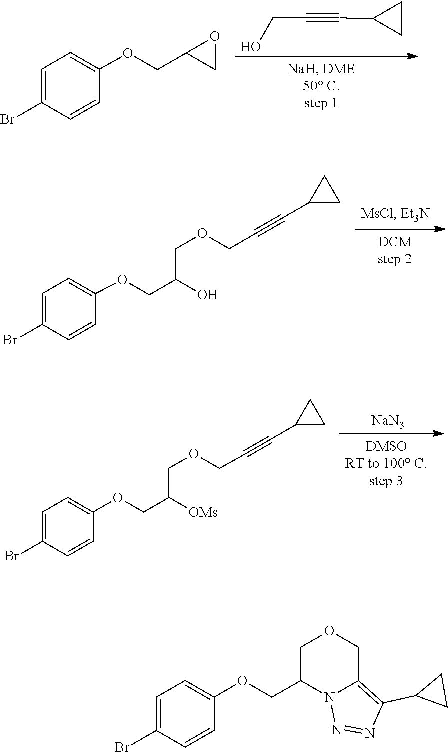

Step 1: To a 40 mL vial was added 3-cyclopropylprop-2-yn-1-ol (525 mg, 5.46 mmol) and dry 1,2-dimethoxyethane (10 mL). The vial was purged with N₂, then NaH (79 mg, 3.27 mmol) was added in one portion, and the mixture stirred at RT for 15 min. 2-((4-bromophenoxy)methyl)oxirane (500 mg, 2.183 mmol) was added, and the reaction was heated to 50° C. for 6 hours. More 3-cyclopropylprop-2-yn-1-ol (105 mg, 1.091 mmol) and NaH (44 mg, 1.09 mmol) were added, and the reaction continued at 50° C. for another 16 hours. The reaction was quenched with AcOH and filtered. To this crude material was added trityl chloride (1.34 g, 4.80 mmol) and triethylamine (2 mL, 14.4 mmol), and the reaction stirred at RT for 3 hours. The crude was then filtered, concentrated, and purified via silica gel chromatography (ISCO, 0 to 30% ethyl acetate/heptane) to give 1-(4-bromophenoxy)-3-((3-cyclopropylprop-2-yn-1-yl)oxy)propan-2-ol, which also contained equal amounts of 3-cyclopropylprop-2-yn-1-ol. The mixture was used in the next step without further purification. LCMS t=0.90 min, m/z=325.4, 327.4 (M+H).

Step 2: To 1-(4-bromophenoxy)-3-((3-cyclopropylprop-2-yn-1-yl)oxy)propan-2-ol (584 mg, 1.73 mmol, 50% purity) in dry DCM (8 mL) was added triethylamine (0.691 mL, 5.20 mmol), followed by dropwise addition of methanesulfonyl chloride (0.404 mL, 5.20 mmol). The reaction was then continued at RT for 30 min. The crude was then directly purified via silica gel chromatography (ISCO, 0 to 30% ethyl acetate/heptane) to give 1-(4-bromophenoxy)-3-((3-cyclopropylprop-2-yn-1-yl)oxy)propan-2-yl methanesulfonate, which contained 30% of an inseparable impurity. The mixture was used in the next step without further purification. LCMS Rt=1.04 min, m/z=403.1, 405.1 (M+H).

Step 3: To a 100 mL flask was added give 1-(4-bromophenoxy)-3-((3-cyclopropylprop-2-yn-1-yl)oxy)propan-2-yl methanesulfonate (515 mg, 1.28 mmol, 70% purity), dry DMSO (10 mL), and sodium azide (208 mg, 3.19 mmol). The reaction was stirred at RT for 16 hours, then heated to 80° C. for 1 hour, and finally heated to 100° C. for 4 hours. The reaction was quenched with excess triphenylphosphine and diluted with water, then stirred at RT for 15 min. The crude was extracted with EtOAc three times, washed with water and brine, dried over sodium sulfate, and concentrated. It was purified via silica gel chromatography (ISCO, 0 to 50% ethyl acetate/heptane) to give 7-((4-bromophenoxy)methyl)-3-cyclopropyl-6,7-dihydro-4H-[1,2,3]triazolo[5,1-c][1,4]oxazine. LCMS Rt=0.90 min, m/z=350.2 352.2 (M+H).

### Example 382: 5-(4-((6-(2-methoxypropan-2-yl)-1,4-dioxan-2-yl)methoxy)phenyl)-2-oxo-6-(trifluoromethyl)-1,2-dihydropyridine-3-carboxamide (382)

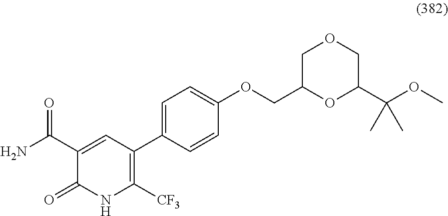 [SMILES: COC(C)(C)C1COCC(COc2ccc(-c3cc(C(N)=O)c(=O)[nH]c3C(F)(F)F)cc2)O1]

5-(4-((6-(2-methoxypropan-2-yl)-1,4-dioxan-2-yl)methoxy)phenyl)-2-oxo-6-(trifluoromethyl)-1,2-dihydropyridine-3-carboxamide (382) was obtained using the procedure described for Example 348, except 2-((4-bromophenoxy)methyl)-6-(difluoromethyl)-1,4-dioxane was replaced with 2-((4-bromophenoxy)methyl)-6-(2-methoxypropan-2-yl)-1,4-dioxane. LCMS Rt=0.77 min, m/z=471.3 (M+H₂O).

### 2-((4-bromophenoxy)methyl)-6-(2-methoxypropan-2-yl)-1,4-dioxane was Obtained Using the Following Procedure

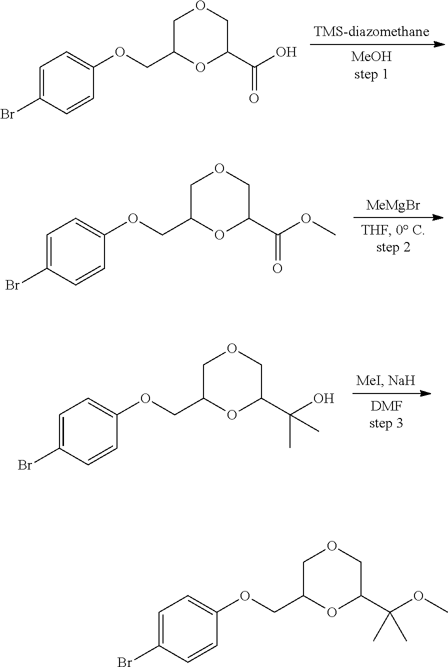 [SMILES: "MeMgBr" ; O=C(O)C1COCC(COc2ccc(Br)cc2)O1 ; COC(=O)C1COCC(COc2ccc(Br)cc2)O1 ; CC(C)(O)C1COCC(COc2ccc(Br)cc2)O1 ; COC(C)(C)C1COCC(COc2ccc(Br)cc2)O1]

Step 1: To a 200 mL flask was added 6-((4-bromophenoxy)methyl)-1,4-dioxane-2-carboxylic acid (756 mg, 2.384 mmol) and dry MeOH (20 mL). Then TMS-diazomethane (7 mL, 14.00 mmol) was added dropwise until bubbling had ceased. The reaction was then continued at RT for 1 hour and then quenched by dropwise addition of acetic acid until bubbling had ceased. The mixture was concentrated and the crude product purified via silica gel chromatography (ISCO, 0 to 40% ethyl acetate/heptane) to give methyl 6-((4-bromophenoxy)methyl)-1,4-dioxane-2-carboxylate. LCMS Rt=0.89 min, m/z=330.9, 332.9 (M+H).

Step 2: To a 100 mL flask was added methyl 6-((4-bromophenoxy)methyl)-1,4-dioxane-2-carboxylate (260 mg, 0.785 mmol) and dry THF (7 mL). The mixture was cooled to 0° C., then a 3M solution of methylmagnesium bromide (0.785 mL, 2.35 mmol) in diethyl ether was added dropwise. The reaction was warmed to RT and stirred for 1 hour. The reaction was quenched at 0° C. with sat. NH₄Cl, extracted with EtOAc twice, dried over sodium sulfate, and concentrated to give crude 2-(6-((4-bromophenoxy)methyl)-1,4-dioxan-2-yl)propan-2-ol which was used in the next step without further purification. LCMS Rt=0.78 min, m/z=313.2, 315.2 (M−H₂O).

Step 3: To a 5 mL microwave vial was added 2-(6-((4-bromophenoxy)methyl)-1,4-dioxan-2-yl)propan-2-ol (140 mg, 0.423 mmol) and dry DMF (3 mL). Dry NaH (32 mg, 1.27 mmol) was added in one portion at RT and stirred for 15 min. Then iodomethane (0.079 ml, 1.27 mmol) was added and the reaction continued at RT for 1 hour. The reaction was quenched with sat. NH₄Cl, extracted with EtOAc twice. The combined organic layers were washed with water, dried over sodium sulfate, and concentrated to give crude product which was purified via silica gel chromatography (ISCO, 0 to 30% ethyl acetate/heptane) to give 2-((4-bromophenoxy)methyl)-6-(2-methoxypropan-2-yl)-1,4-dioxane. LCMS Rt=0.92 min, m/z=313.3, 315.3 (M-CH₃OH).

### Example 383: 5-(4-((6-(3-fluoropentan-3-yl)-1,4-dioxan-2-yl)methoxy)phenyl)-2-oxo-6-(trifluoromethyl)-1,2-dihydropyridine-3-carboxamide (383)

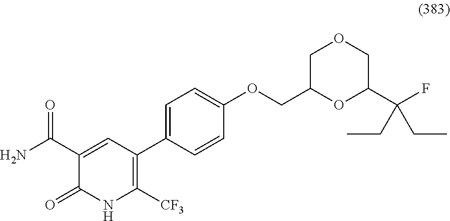 [SMILES: CCC(F)(CC)C1COCC(COc2ccc(-c3cc(C(N)=O)c(=O)[nH]c3C(F)(F)F)cc2)O1]

5-(4-((6-(3-fluoropentan-3-yl)-1,4-dioxan-2-yl)methoxy)phenyl)-2-oxo-6-(trifluoromethyl)-1,2-dihydropyridine-3-carboxamide (383) was obtained using the procedure described for Example 348, except 2-((4-bromophenoxy)methyl)-6-(difluoromethyl)-1,4-dioxane was replaced with 2-((4-bromophenoxy)methyl)-6-(3-fluoropentan-3-yl)-1,4-dioxane. LCMS Rt=4.30, 4.31 min, m/z=487.3 (M+H).

### 2-((4-bromophenoxy)methyl)-6-(3-fluoropentan-3-yl)-1,4-dioxane was Obtained Using the Following Procedure

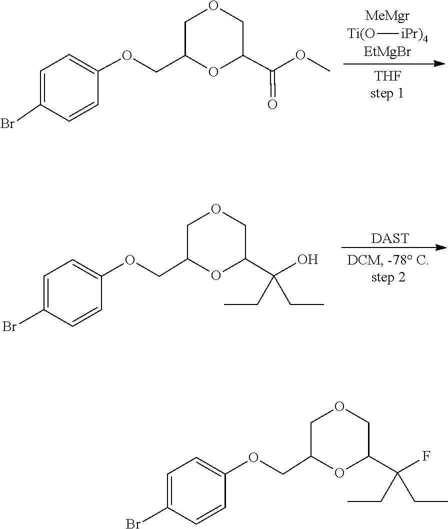

Step 1: An oven-dried 5 mL microwave vial was cooled under N₂, and to it was added dry THF (3 mL), and titanium(IV) isopropoxide (0.085 mL, 0.287 mmol). Then methylmagnesium bromide (3M solution in diethyl ether, 0.15 mL, 0.45 mmol) was added dropwise at RT. The reaction was cooled to 0° C., and methyl 6-((4-bromophenoxy)methyl)-1,4-dioxane-2-carboxylate (95 mg, 0.287 mmol) was added, followed by dropwise addition of ethylmagnesium bromide (1M solution in THF, 0.46 mL, 0.46 mmol) over 2 minutes. The reaction was warmed to RT and stirred for 1 hour. Then it was quenched with sat. NH₄Cl and stirred for 5 min, then filtered, extracted with EtOAc twice, dried over sodium sulfate, and concentrated. The crude was then purified via silica gel chromatography (ISCO, 0 to 100% ethyl acetate/heptane) to give 3-(6-((4-bromophenoxy)methyl)-1,4-dioxan-2-yl)pentan-3-ol. LCMS Rt=0.89 min, m/z=341.4, 341.3 (M−H₂O).

Step 2: A 50 mL flask with give 3-(6-((4-bromophenoxy)methyl)-1,4-dioxan-2-yl)pentan-3-ol (62.7 mg, 0.175 mmol) in dry DCM (2 mL) was cooled to −78° C. under a nitrogen atmosphere. To it was added diethylaminosulfur trifluoride (0.046 mL, 0.349 mmol) at −78° C., and the reaction continued at −78° C. for 2 hours. The reaction was quenched with sat. NaHCO₃ at −78° C., then allowed to warm to RT overnight. The reaction was extracted with DCM, dried over sodium sulfate, and concentrated. The crude was then purified via silica gel chromatography (ISCO, 0 to 100% ethyl acetate/heptane) to give 2-((4-bromophenoxy)methyl)-6-(3-fluoropentan-3-yl)-1,4-dioxane. LCMS Rt=1.04 min, m/z=341.3, 343.3 (M−HF).

### Example 384: 5-(4-((6-(hydroxymethyl)-1,4-dioxan-2-yl)methoxy)phenyl)-2-oxo-6-(trifluoromethyl)-1,2-dihydropyridine-3-carboxamide (384)

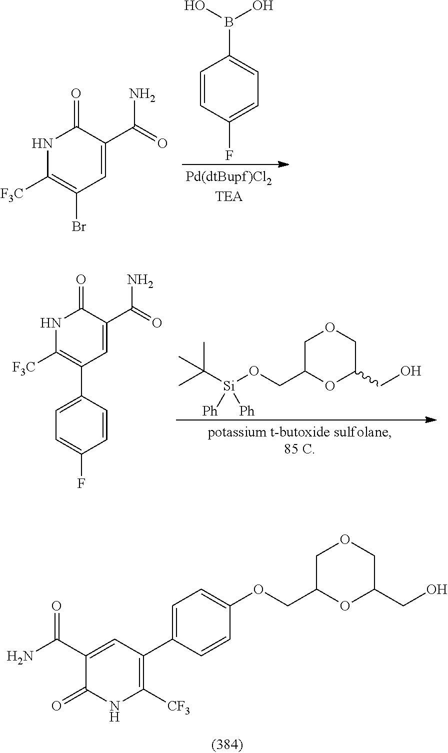

Step 1: To a 40 mL vial with a stirbar was added 5-bromo-2-oxo-6-(trifluoromethyl)-1,2-dihydropyridine-3-carboxamide (1500 mg, 5.26 mmol), (4-fluorophenyl)boronic acid (810 mg, 5.79 mmol) and Pd(dtBupf)Cl₂ (68.6 mg, 0.105 mmol). The vial was sealed with a septa-top cap and purged with vac/N₂ (×3). TPGS-750 2 w % (17.5 mL) was added followed by triethylamine (3.7 mL, 26.3 mmol). The reaction was warmed to 50° C. and stirred vigorously. The reaction mixture was acidified with 2M KHSO₄, diluted with H₂O and filtered through glass wool. The product was removed from the wool using MeOH and the eluent concentrated. The resultant solid was suspended in DCM, filtered, washed with Et₂O and dried under vacuum to give 5-(4-fluorophenyl)-2-oxo-6-(trifluoromethyl)-1,2-dihydropyridine-3-carboxamide which was used without further purification.

Step 2: To an oven dried 4 mL vial with a stirbar was added 5-(4-fluorophenyl)-2-oxo-6-(trifluoromethyl)-1,2-dihydropyridine-3-carboxamide (40 mg, 0.133 mmol), potassium tert-butoxide (44.9 mg, 0.400 mmol) and (6-(((tert-butyldiphenylsilyl)oxy)methyl)-1,4-dioxan-2-yl)methanol (77 mg, 0.200 mmol). The vial was sealed with a septa-top cap and purged with vac/N₂ (×3). Sulfolane (266 μL) was added and the reaction was heated to 85° C. and stirred for 96 h. The reaction was diluted with Et₂O, the layers separated and the organic layer washed with water, then brine, dried over Na₂SO₄, filtered and concentrated to give 5-(4-((6-(hydroxymethyl)-1,4-dioxan-2-yl)methoxy)phenyl)-2-oxo-6-(trifluoromethyl)-1,2-dihydropyridine-3-carboxamide (383). LCMS t=0.59 min, m/z=429.2 [M+H]. ¹H NMR (500 MHz, DMSO-d6) δ 13.63 (br. s, 1H), 8.46 (br. s, 1H), 8.28 (s, 1H), 8.17 (br. s, 1H), 7.27 (d, J=8.3 Hz, 2H), 7.11-6.95 (m, 2H), 4.73 (br. s, 1H), 4.17 (ddd, J=30.2, 10.1, 5.9 Hz, 2H), 4.07-4.01 (m, 1H), 3.78-3.67 (m, 3H), 3.62 (dd, J=11.7, 4.9 Hz, 1H), 3.55-3.46 (m, 4H).

### (6-(((tert-butyidiphenylsilyl)oxy)methyl)-1,4-dioxan-2-yl)methanol was Obtained Using the Following Procedure

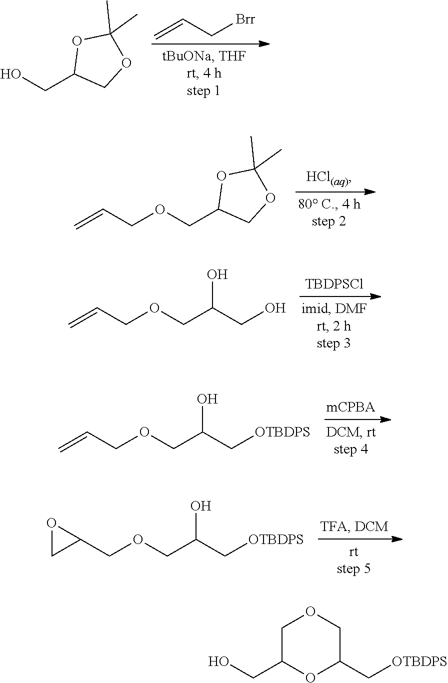

Step 1: To an oven-dried 1 L 3-necked flask with a stirbar and an internal thermometer was added sodium tert-butoxide (11.87 g, 124 mmol) and the vessel was purged with vac/N₂×3. Anhydrous THF (475 mL) was added and the reaction was cooled to 5° C. in an ice bath. (2,2-dimethyl-1,3-dioxolan-4-yl)methanol (11.8 mL, 95 mmol) was added dropwise over 5 mins, during which time the internal temperature rose to 8° C. The reaction was stirred for 10 mins and allyl bromide (10.7 mL, 124 mmol) was added dropwise. The reaction was then warmed to rt and stirred for 3.5 h. The reaction mixture was poured into water and extracted into EA (25 mL×3). The combined organic layers were washed with brine, dried over Na₂SO₄, filtered and concentrated in vacuo to give 4-((allyloxy)methyl)-2,2-dimethyl-1,3-dioxolane which was used without further purification.

Step 2: To a 250 mL flask with a stirbar was added 4-((allyloxy)methyl)-2,2-dimethyl-1,3-dioxolane (16.4 g, 95 mmol) and 2N HCl (50 mL). A reflux condenser was added and the reaction was heated to 50° C. and stirred vigorously overnight. The reaction was concentrated in vacuo and azeotroped with benzene multiple times to remove all water and obtain 3-(allyloxy)propane-1,2-diol. ¹H NMR (500 MHz, Chloroform-d) δ 5.97-5.81 (m, 1H), 5.28 (d, J=17.5 Hz, 1H), 5.21 (d, J=10.2 Hz, 1H), 4.02 (d, J=5.7 Hz, 2H), 3.88 (p, J=5.2 Hz, 1H), 3.72 (dd, J=11.4, 3.9 Hz, 1H), 3.67-3.61 (m, 1H), 3.58-3.47 (m, 2H), 2.38 (br. s, 2H).

Step 3: To an oven dried 250 mL flask with a stirbar was added 3-(allyloxy)propane-1,2-diol (11.16 g, 84.0 mmol) and imidazole (13.8 g, 203 mmol). The flask was purged with vac/N₂×3 and DMF (94 mL) was added before cooling the reaction to 0° C. in an ice bath. Tert-butyldiphenylsilyl chloride (23.9 mL, 93 mmol) was then added dropwise over 15 mins and the reaction was warmed to rt and stirred for 2 h. The reaction was diluted with heptane (1.5 L) and the solution was washed with 1:1 water/brine×2. The organic layer was dried over Na₂SO₄, filtered and concentrated to in vacuo to give 1-(allyloxy)-3-((tert-butyldiphenylsilyl)oxy)propan-2-ol. LCMS t=1.20 min, m/z=393.3 [M+Na].

Step 4: To a 40 mL vial with a stirbar was added 1-(allyloxy)-3-((tert-butyldiphenylsilyl)oxy)propan-2-ol (5.00 g, 13.49 mmol), DCM (27.0 mL) and mCPBA (77 w %, 4.23 g, 18.89 mmol) and the reaction stirred at rt for 2 h. Saturated Na₂S2O₃a was added and the reaction stirred vigorously for 5 mins. The reaction was diluted with Et₂O and washed with Na₂S2O₃₈, NaHCO_{3(aq)}×2, brine and then dried over Na₂SO₄, filtered and concentrated in vacuo to give 1-((tert-butyldiphenylsilyl)oxy)-3-(oxiran-2-ylmethoxy)propan-2-ol which was used without further purification. LCMS t=1.09 min, m/z=409.3 [M+Na]

Step 5: To an oven dried 100 ml RBF with a stirbar was 1-((tert-butyldiphenylsilyl)oxy)-3-(oxiran-2-ylmethoxy)propan-2-ol (1300 mg, 3.36 mmol), DCM (67.3 mL) and TFA (130 μl, 1.68 mmol); the reaction was stirred at rt overnight. Additional TFA (130 μl, 1.62 mmol) was added and the reaction stirred for 5 h. 15% NaOH was added to basify the reaction and the inhomogeneous mixture stirred vigorously. After 30 the reaction was diluted with Et₂O, the layers separated and the organic layer washed with water, then brine, dried over Na₂SO₄ and filtered. SiO₂ was added to the eluent, which was then concentrated in vacuo. The resultant solid was then loaded onto a plug of SiO₂ and washed with heptane. The product was eluted by washing with 1:1 EA/heptane and concentrated to give (6-(((tert-butyldiphenylsilyl)oxy)methyl)-1,4-dioxan-2-yl)methanol. LCMS t=1.09 min, 1.10 min, m/z=427.2 [M+H₂O+Na].

### Example 385: 5-(4-((6-((3-fluoroazetidin-1-yl)methyl)-1,4-dioxan-2-yl)methoxy)phenyl)-2-oxo-6-(trifluoromethyl)-1,2-dihydropyridine-3-carboxamide (385)

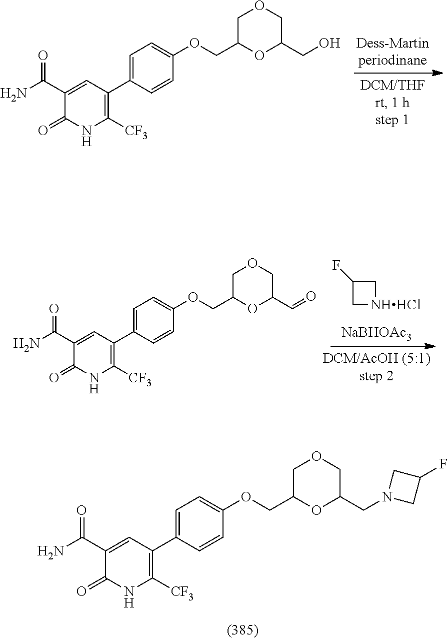

Step 1: To an 8 mL vial was added 5-(4-((6-(hydroxymethyl)-1,4-dioxan-2-yl)methoxy)phenyl)-2-oxo-6-(trifluoromethyl)-1,2-dihydropyridine-3-carboxamide (383) (86 mg, 0.20 mmol) and Dess-Martin Periodinane (102 mg, 0.241 mmol). THF (1.0 mL) and DCM (1.0 mL) were added and the reaction agitated vigorously to give a homogenous solution which was stirred at rt. after 30 mins. The reaction was quenched by addition of a drop of MeOH. After stirring for 5 mins, the reaction was concentrated in vacuo, then suspended in DCM and filtered using a syringe filter. The eluent was concentrated to give 5-(4-((6-formyl-1,4-dioxan-2-yl)methoxy)phenyl)-2-oxo-6-(trifluoromethyl)-1,2-dihydropyridine-3-carboxamide which was used without further purification. LCMS t=0.61 min, m/z=427.4 [M+H].

Step 2: To a 1 mL vial with stirbar was added 3-fluoroazetidine hydrochloride (8.4 mg, 0.075 mmol) and a solution of 5-(4-((6-formyl-1,4-dioxan-2-yl)methoxy)phenyl)-2-oxo-6-(trifluoromethyl)-1,2-dihydropyridine-3-carboxamide (21.3 mg, 0.05 mmol) in DCM (417 μl) and AcOH (83 μl). The reaction was stirred for 10 mins and NaBH(OAc)₃ (31.8 mg, 0.15 mmol) was then added, giving a suspension which was stirred overnight. Purification by reverse phase HPLC followed by lyophilization gave 5-(4-((6-((3-fluoroazetidin-1-yl)methyl)-1,4-dioxan-2-yl)methoxy)phenyl)-2-oxo-6-(trifluoromethyl)-1,2-dihydropyridine-3-carboxamide (385) as a racemic mixture of diastereomers. LCMS t=0.61 min, m/z=486.4 [M+H].

### Example 386: 5-(4-((6-((3,3-difluoroazetidin-1-yl)methyl)-1,4-dioxan-2-yl)methoxy)phenyl)-2-oxo-6-(trifluoromethyl)-1,2-dihydropyridine-3-carboxamide (386)

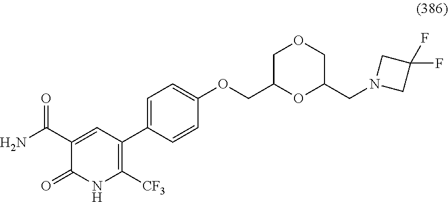 [SMILES: NC(=O)c1cc(-c2ccc(OCC3COCC(CN4CC(F)(F)C4)O3)cc2)c(C(F)(F)F)[nH]c1=O]

5-(4-((6-((3,3-difluoroazetidin-1-yl)methyl)-1,4-dioxan-2-yl)methoxy)phenyl)-2-oxo-6-(trifluoromethyl)-1,2-dihydropyridine-3-carboxamide (386) was obtained using the method described for the synthesis of 5-(4-((6-((3-fluoroazetidin-1-yl)methyl)-1,4-dioxan-2-yl)methoxy)phenyl)-2-oxo-6-(trifluoromethyl)-1,2-dihydropyridine-3-carboxamide (385), except 3-fluoroazetidine hydrochloride was replaced with 3,3-difluoroazetidine. LCMS t=0.62 min, m/z=504.3 [M+H].

### Example 387: 5-(4-((6-((3-methoxyazetidin-1-yl)methyl)-1,4-dioxan-2-yl)methoxy)phenyl)-2-oxo-6-(trifluoromethyl)-1,2-dihydropyridine-3-carboxamide (387)

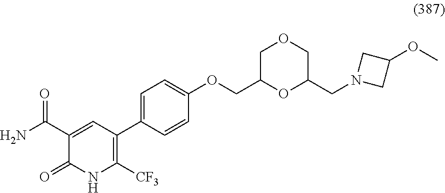 [SMILES: COC1CN(CC2COCC(COc3ccc(-c4cc(C(N)=O)c(=O)[nH]c4C(F)(F)F)cc3)O2)C1]

5-(4-((6-((3-methoxyazetidin-1-yl)methyl)-1,4-dioxan-2-yl)methoxy)phenyl)-2-oxo-6-(trifluoromethyl)-1,2-dihydropyridine-3-carboxamide (387) was obtained using the method described for the synthesis of 5-(4-((6-((3-fluoroazetidin-1-yl)methyl-1,4-dioxan-2-yl)methoxy)phenyl)-2-oxo-6-(trifluoromethyl)-1,2-dihydropyridine-3-carboxamide (385), except 3-fluoroazetidine hydrochloride was replaced with 3-methoxyazetidine LCMS t=0.62, m/z=498.4 [M+H].

### Example 388: 5-(4-((2-fluoro-1,4-dioxan-2-yl)methoxy)phenyl)-2-oxo-6-(trifluoromethyl)-1,2-dihydropyridine-3-carboxamide (388)

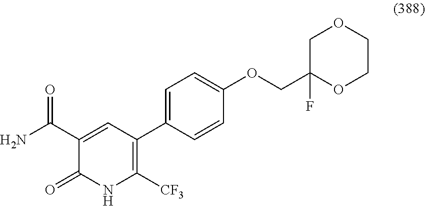 [SMILES: NC(=O)c1cc(-c2ccc(OCC3(F)COCCO3)cc2)c(C(F)(F)F)[nH]c1=O]

5-(4-((2-fluoro-1,4-dioxan-2-yl)methoxy)phenyl)-2-oxo-6-(trifluoromethyl)-1,2-dihydropyridine-3-carboxamide (388) was obtained using the procedure described for Example 348, except 2-((4-bromophenoxy)methyl)-6-(difluoromethyl)-1,4-dioxane was replaced with 2-((4-bromophenoxy)methyl)-2-fluoro-1,4-dioxane. ¹H NMR (400 MHz, DMSO-d6) δ=9.08 (br s, 1H), 8.55 (s, 1H), 7.24 (d, J=8.4 Hz, 2H), 6.98 (d, J=8.8 Hz, 2H), 5.98 (br s, 1H), 4.39-4.26 (m, 1H), 4.18-3.97 (m, 3H), 3.94-3.65 (m, 4H). LCMS: Rt=0.74 min, m/z=417.0 (M+H).

### 2-((4-bromophenoxy)methyl)-2-fluoro-1,4-dioxane was Obtained Using the Following Procedure

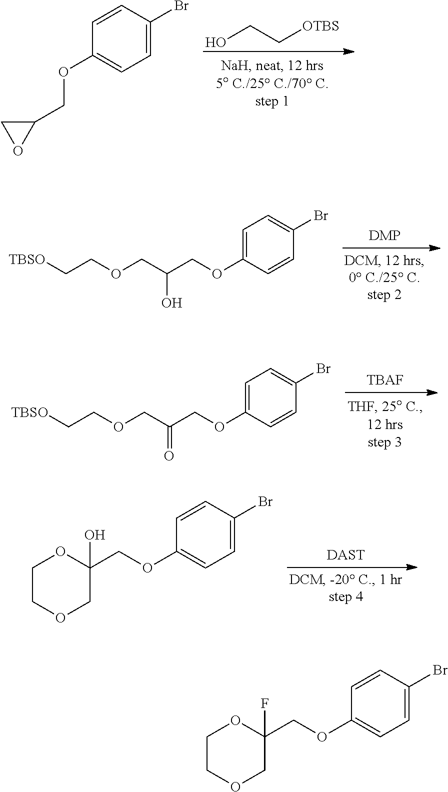

Step 1: To a solution of 2-((tert-butyldimethylsilyl)oxy)ethan-1-ol (61.5 g, 349.3 mmol) was added NaH (5.6 g, 140 mmol) in portions at 5° C. The mixture was stirred at 25° C. for 0.5 h. Then 2-((4-bromophenoxy)methyl)oxirane (16 g, 69.8 mmol) was added to the mixture at 25° C. and the mixture was stirred at 70° C. for 11.5 hrs. The mixture was washed with H₂O (200 mL) and extracted with EA (200 mL×3). The combined organic layer was washed with brine, dried over Na₂SO₄, filtered and concentrated. The residue was purified by column chromatography (PE/EA=10/1) to give 1-(4-bromophenoxy)-3-(2-((tert-butyldimethylsilyloxy)ethoxy)propan-2-ol. ¹H NMR (400 MHz, CDCl3) δ 7.43-7.33 (m, 2H), 6.86-6.74 (m, 2H), 4.20-4.09 (m, 1H), 4.01-3.98 (m, 2H), 3.81-3.76 (m, 2H), 3.74-3.60 (m, 4H), 2.91 (br s, 1H), 0.87 (s, 9H), 0.11 (s, 6H).

Step 2: A solution of 1-(4-bromophenoxy)-3-(2-((tert-butyldimethylsilyl)oxy)ethoxy)propan-2-ol (6.5 g, 16.03 mmol) in DCM (100 mL) was added Dess-Martin periodane (10.2 g, 24.05 mmol) at 0° C. The mixture was stirred 25° C. for 12 hrs. The residue was purified by column (PE) directly to give 1-(4-bromophenoxy)-3-(2-((tert-butyldimethylsilyl)oxy)ethoxy)propan-2-one. LCMS: Rt=1.208 min, [M+H]+=403.2. ¹H NMR (400 MHz, CDCl3) δ 7.46-7.34 (m, 2H), 6.86-6.74 (m, 2H), 4.81 (s, 2H), 4.40 (s, 2H), 3.88-3.78 (m, 2H), 3.71-3.59 (m, 2H), 0.91 (s, 9H), 0.08 (s, 6H).

Step 3: To a solution of 1-(4-bromophenoxy)-3-(2-((tert-butyldimethylsilyl)oxy)ethoxy)propan-2-one (5.5 g, 13.65 mmol) in THF (55 mL) was added TBAF (27.3 mL, 27.3 mmol) at 25° C. The mixture was stirred 25° C. for 12 hrs. The mixture was concentrated. The residue was purified by column (PE/EA=2/1) to give 2-((4-bromophenoxy)methyl)-1,4-dioxan-2-ol. ¹H NMR (400 MHz, CDCl3) δ 7.39 (d, J=9.0 Hz, 2H), 6.83 (d, J=9.0 Hz, 2H), 4.25 (m, 1H), 4.01-3.62 (m, 7H), 3.50 (br s, 1H).

Step 4: To a solution of 2-((4-bromophenoxy)methyl)-1,4-dioxan-2-ol (150 mg, 0.519 mmol) in DCM (2 mL) was added DAST (279 mg, 2.595 mmol) at −20° C. Then the mixture was stirred at −20° C. for 1 h. The mixture was diluted with ethanol (10 mL) and quenched with NaHCO₃ until the pH=8, then the mixture was filtered The filtrate was concentrated in vacuo and the residue purified with Prep-TLC (PE:EA=3:1) to give 2-((4-bromophenoxy)methyl)-2-fluoro-1,4-dioxane. TLC: PE/EA=3:1, Rf=0.8. ¹H NMR (400 MHz, CHLOROFORM-d) δ=7.45-7.33 (m, 2H), 6.89-6.76 (m, 2H), 4.35-4.25 (m, 1H), 4.13-4.04 (m, 1H), 4.03-3.91 (m, 2H), 3.90-3.64 (m, 4H), 3.31-3.10 (m, 1H). 19F NMR (376 MHz, CHLOROFORM-d) δ=−122.76-−128.13 (m, 1F).

### Example 389: 5-(4-((1,4-dioxan-2-yl)difluoromethoxy)phenyl)-2-oxo-6-(trifluoromethyl)-1,2-dihydropyridine-3-carboxamide (389)

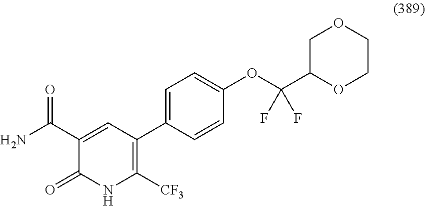 [SMILES: NC(=O)c1cc(-c2ccc(OC(F)(F)C3COCCO3)cc2)c(C(F)(F)F)[nH]c1=O]

5-(4-((1,4-dioxan-2-yl)difluoromethoxy)phenyl)-2-oxo-6-(trifluoromethyl)-1,2-dihydropyridine-3-carboxamide (389) was obtained using the procedure described for Example 348, except 2-((4-bromophenoxy)methyl)-6-(difluoromethyl)-1,4-dioxane was replaced with 2-((4-bromophenoxy)difluoromethyl)-1,4-dioxane. ¹H NMR (400 MHz, DMSO+D2O) δ=8.18 (s, 1H), 8.00 (s, 1H), 7.54 (d, J=8.0 Hz, 2H), 7.37 (d, J=8.0 Hz, 2H), 3.68-3.53 (m, 5H), 3.49-3.37 (m, 2H), 3.27-3.18 (m, 1H), 2.33 (s, 2H). LCMS: Rt=0.76 min, m/z=433.1 (M+H).

### 2-((4-bromophenoxy)difluoromethyl)-1,4-dioxane was Obtained Using the Following Procedure

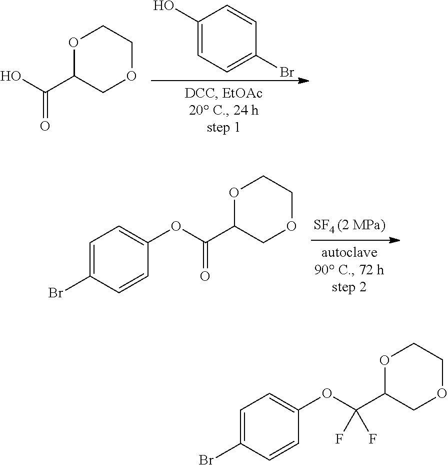 [SMILES: O=C(O)C1COCCO1 ; Oc1ccc(Br)cc1 ; O=C(Oc1ccc(Br)cc1)C1COCCO1 ; FC(F)(Oc1ccc(Br)cc1)C1COCCO1]

Step 1: To a mixture of 1,4-dioxane-2-carboxylic acid (1 g, 7.57 mmol), 4-bromophenol (1.44 g, 8.33 mmol) was added DCC (1.56 g, 7.57 mmol in EA (40 mL). The reaction was stirred at 20° C. for 24 h. The reaction mixture was filtered and concentrated in vacuo. The residue was purified by silica gel column chromatography (PE:EA=20:1 to 10:1) to give 4-bromophenyl 1,4-dioxane-2-carboxylate. TLC: (PE:EA=5:1, Rf=0.4). ¹H NMR (400 MHz, CHLOROFORM-d) δ=7.54-7.48 (m, 2H), 7.04-6.98 (m, 2H), 4.49 (dd, J=3.3, 7.8 Hz, 1H), 4.17-4.04 (m, 3H), 3.93-3.69 (m, 5H).

Step 2: SF4 was bubbled into the autoclave containing 0.6 g of 4-bromophenyl 1,4-dioxane-2-carboxylate at −70° C. for 1 min. Then the autoclave was stirred at 90° C. for 3 day. TLC (PE:EA=5:1, Rf=0.4) showed SM was remaining and a new spot (Rf=0.5) was formed. The mixture was concentrated in vacuo. The residue was purified by column chromatography over silica gel (eluent: PE:EA=5:1) to give 2-((4-bromophenoxy)difluoromethyl)-1,4-dioxane. TLC: (PE:EA=5:1, Rf=0.5). ¹H NMR (400 MHz, CHLOROFORM-d) δ=7.50-7.44 (m, 2H), 7.08 (d, J=8.9 Hz, 2H), 4.09-3.66 (m, 8H).

### Example 390: 5-(4-((cis-7,7-difluoro-2,5-dioxabicyclo[4.1.0]heptan-3-yl)methoxy)phenyl)-2-oxo-6-(trifluoromethyl)-1,2-dihydropyridine-3-carboxamide (390)

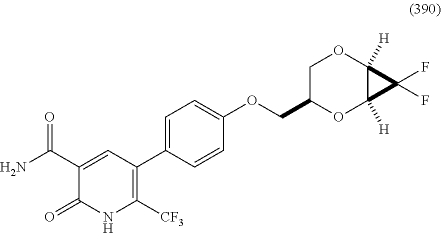

5-(4-((cis-7,7-difluoro-2,5-dioxabicyclo[4.1.0]heptan-3-yl)methoxy)phenyl)-2-oxo-6-(trifluoromethyl)-1,2-dihydropyridine-3-carboxamide (390) was obtained using the procedure described for Example 348, except 2-((4-bromophenoxy)methyl)-6-(difluoromethyl)-1,4-dioxane was replaced with cis-3-((4-bromophenoxy)methyl)-7,7-difluoro-2,5-dioxabicyclo[4.1.0]heptane. LCMS t=0.79, m/z=447.5 [M+H].

### cis-3-((4-bromophenoxy)methyl)-7,7-difluoro-2,5-dioxabicyclo[4.1.0]heptane and trans-3-((4-bromophenoxy)methyl)-7,7-difluoro-2,5-dioxabicyclo[4.1.0]heptane were Obtained Using the Following Procedure

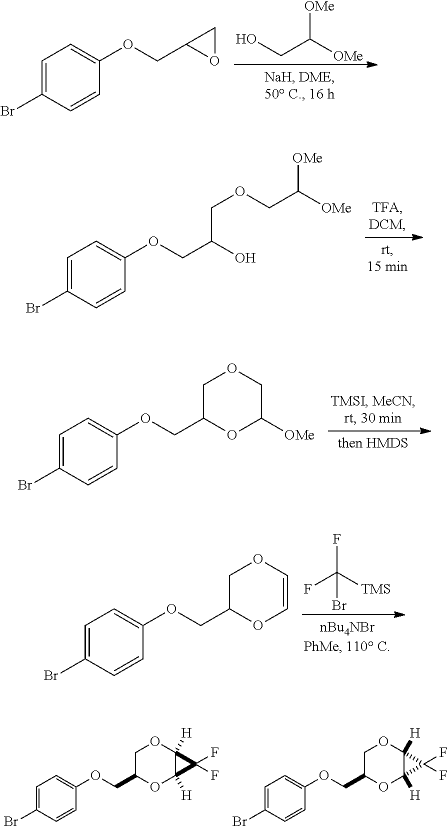

Step 1: To an oven-dried 40 mL vial with a stirbar was added sodium hydride (60% in mineral oil, 262 mg, 6.55 mmol). The vial was sealed with a septa-top cap and purged with vac/N₂ (×3). DME (19 mL) was added, followed by dropwise addition of 2,2-dimethoxyethanol (1158 mg, 10.9 mmol). The reaction was stirred for 20 mins forming a white suspension to which was added 2-((4-bromophenoxy)methyl)oxirane (1000 mg, 4.37 mmol) in a single portion. The reaction was heated to 50° C. and the solution stirred overnight. The reaction mixture was stirred at 50° C. for a further 24 h. The reaction was quenched with MeOH and the mixture filtered through a plug of SiO₂ with EA to give 1-(4-bromophenoxy)-3-(2,2-dimethoxyethoxy)propan-2-ol which was used without further purification. LCMS t=0.80, m/z=357.2, 359.2 [M+Na].

Step 2: To a 4 mL vial with a stirbar was added 1-(4-bromophenoxy)-3-(2,2-dimethoxyethoxy)propan-2-ol (1.26 g, 3.76 mmol), DCM (15.7 ml) and TFA (3.13 ml). The reaction was stirred at rt for 15 min. The reaction was concentrated in vacuo to give 2-((4-bromophenoxy)methyl)-6-methoxy-1,4-dioxane which was used without further purification. LCMS t=0.89, 0.92 m/z=271.1, 273.1 [M−MeO].

Step 3: To a 20 mL vial with a stirbar was added 2-((4-bromophenoxy)methyl)-6-methoxy-1,4-dioxane (1140 mg, 3.76 mmol). The vial was sealed with a septa-top cap and purged with vac/N₂ (×3). MeCN (37.6 mL) was added, followed by TMSI (3.1 mL, 22.6 mmol) and the mixture stirred at rt for 30 min. HMDS (11.8 mL, 56.4 mmol) was added and the reaction stirred at rt for 15 mins. The reaction was quenched by the addition of NaHCO_{3(aq)}. The resulting mixture was extracted into EA×3, dried over Na₂SO₄, filtered and concentrated in vacuo to yield 2-((4-bromophenoxy)methyl)-2,3-dihydro-1,4-dioxine. LCMS t=0.98, m/z=271.1, 273.1 [M+H].

Step 4: To a 5 mL oven dried MW vial with a stirbar was added 2-((4-bromophenoxy)methyl)-2,3-dihydro-1,4-dioxine (50 mg, 0.184 mmol) and TBAB (1.8 mg, 5.5 μmol). The vial was sealed and purged with vac/N₂ (×3). Toluene (0.74 mL) and (bromodifluoromethyl)trimethylsilane (43 μl, 0.28 mmol) were added and the reaction heated to 110° C. and stirred for 4 h. An additional equivalent of (bromodifluoromethyl)trimethylsilane was added and the reaction heated to 100° C. for 3 h. The reaction was concentrated in vacuo and purified by silica gel chromatography (ISCO, 0-30% EA/Hept) to give cis-3-((4-bromophenoxy)methyl)-7,7-difluoro-2,5-dioxabicyclo[4.1.0]heptane and trans-3-((4-bromophenoxy)methyl)-7,7-difluoro-2,5-dioxabicyclo[4.1.0]heptane. LCMS t=1.03, m/z=321.1, 323.1 [M+H].

### Example 391: 5-(4-((trans-7,7-difluoro-2,5-dioxabicyclo[4.1.0]heptan-3-yl)methoxy)phenyl)-2-oxo-6-(trifluoromethyl)-1,2-dihydropyridine-3-carboxamide (391)

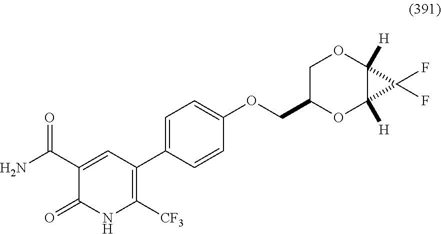 [SMILES: NC(=O)c1cc(-c2ccc(OC[C@H]3CO[C@H]4[C@@H](O3)C4(F)F)cc2)c(C(F)(F)F)[nH]c1=O]

5-(4-((trans-7,7-difluoro-2,5-dioxabicyclo[4.1.0]heptan-3-yl)methoxy)phenyl)-2-oxo-6-(trifluoromethyl)-1,2-dihydropyridine-3-carboxamide (391) was obtained using the procedure described for Example 348, except 2-((4-bromophenoxy)methyl)-6-(difluoromethyl)-1,4-dioxane was replaced with trans-3-((4-bromophenoxy)methyl)-7,7-difluoro-2,5-dioxabicyclo[4.1.0]heptane. LCMS t=0.81, m/z=447.5 [M+H]. ¹H NMR (500 MHz, DMSO-d6) δ 13.63 (br. s, 1H), 8.46 (br. s, 1H), 8.28 (s, 1H), 8.18 (br. s, 1H), 7.33-7.21 (m, 2H), 7.10-7.01 (m, 2H), 4.17-4.02 (m, 5H), 3.91 (dd, J=11.6, 2.7 Hz, 1H), 3.65-3.51 (m, 1H).

### Examples 392: 5-(4-((6-(methoxymethyl)-5-methyl-1,4-dioxan-2-yl)methoxy)phenyl)-2-oxo-6-(trifluoromethyl)-1,2-dihydropyridine-3-carboxamide (Peak 1) (392)

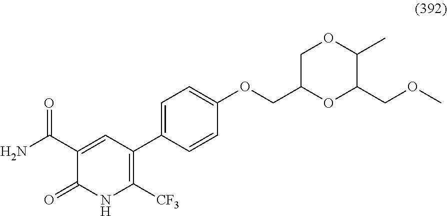 [SMILES: COCC1OC(COc2ccc(-c3cc(C(N)=O)c(=O)[nH]c3C(F)(F)F)cc2)COC1C]

5-(4-((6-methoxy-5-methyl-1,4-dioxan-2-yl)methoxy)phenyl)-2-oxo-6-(trifluoromethyl)-1,2-dihydropyridine-3-carboxamide (peak 1) (392) was obtained using the procedure described for Example 348, except 2-((4-bromophenoxy)methyl)-6-(difluoromethyl)-1,4-dioxane was replaced with 5-((4-bromophenoxy)methyl)-3-(methoxymethyl)-2-methyl-1,4-dioxane. Purification by reverse phase HPLC gave a racemic single diastereomer of undefined stereochemistry. LCMS t=0.77, m/z=457.4 [M+H]. Peak 1: ¹H NMR (500 MHz, DMSO-d6) δ 13.65 (br. s, 1H), 8.46 (br. s, 1H), 8.27 (s, 1H), 8.17 (br. s, 1H), 7.33-7.17 (m, 2H), 7.11-6.96 (m, 2H), 4.07-3.97 (m, 2H), 3.97-3.90 (m, 2H), 3.78 (qd, J=6.6, 3.0, 1H), 3.64 (t, J=11.2 Hz, 1H), 3.57 (dd, J=11.7, 3.3 Hz, 1H), 3.25 (s, 3H), 3.25-3.21 (m, 2H), 1.15 (d, J=6.7 Hz, 3H).

### 5-((4-bromophenoxy)methyl)-3-(methoxymethyl)-2-methyl-1,4-dioxane was Obtained Using the Following Procedure

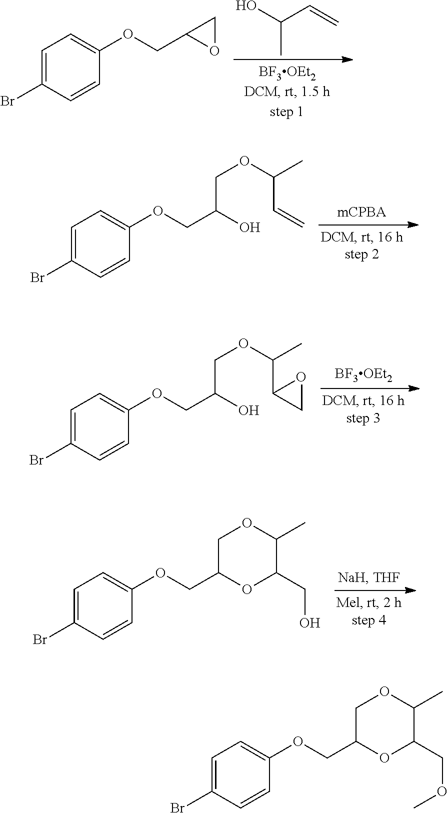

Step 1: To an oven dried 8 mL vial with a stirbar was added 2-((4-bromophenoxy)methyl)oxirane (250 mg, 1.09 mmol). The vial was sealed with a septa-top cap and purged with vac/N₂ (×3). DCM (5.5 mL) was added, followed by but-3-en-2-ol (142 μL, 1.64 mmol). BF₃·OEt₂ (28 μl, 0.22 mmol) was added dropwise at rt and the reaction was stirred at rt for 1.5 h. Solid K₂CO₃ and MeOH were added and the reaction was stirred for 5 mins before filtering through a plug of SiO₂ with EA. The eluent was concentrated in vacuo and purified by silica gel chromatography (ISCO, 0-60% EA/heptane) to give 1-(4-bromophenoxy)-3-(but-3-en-2-yloxy)propan-2-ol. LCMS t=0.96 min, m/z=301.1, 303.2 [M+H].

Step 2: To a 4 mL vial with a stirbar was added 1-(4-bromophenoxy)-3-(but-3-en-2-yloxy)propan-2-ol (162 mg, 0.54 mmol), DCM (1.35 mL) and mCPBA (77 w %, 157 mg, 0.70 mmol) and the reaction stirred overnight at rt. Na₂S₂O_{3(aq)} was added and the reaction stirred vigorously for 5 mins. The reaction was diluted with Et₂O, washed sequentially with Na2S₂O_{3(aq)}, then NaHCO_{3(aq)} ((×2), brine, dried over Na₂SO₄, filtered through a plug of SiO₂ and concentrated in vacuo to give 1-(4-bromophenoxy)-3-(1-(oxiran-2-yl)ethoxy)propan-2-ol which was used without further purification. LCMS t=0.81, 0.82 min, m/z=319.2 [M+H].

Step 3: To an oven-dried 20 mL vial with a stirbar was added 1-(4-bromophenoxy)-3-(1-(oxiran-2-yl)ethoxy)propan-2-ol (144 mg, 0.45 mmol). The vial was sealed with a septa-top cap and purged with vac/N₂ (×3). DCM (9.1 mL) was added, followed by BF₃—OEt₂ (11.5 μl, 0.09 mmol) and the reaction was then stirred at rt overnight. The reaction was quenched by the addition of K₂CO₃ and MeOH and then filtered through a plug of SiO₂ with EA. Purification by silica gel chromatography (ISCO, (0-50% EA/heptane) gave (6-((4-bromophenoxy)methyl)-3-methyl-1,4-dioxan-2-yl)methanol which slowly crystallised under high vacuum. LCMS t=0.78, 0.79 min, m/z=319.2 [M+H].

Step 4: To a 4 mL vial with a stirbar was added (6-((4-bromophenoxy)methyl)-3-methyl-1,4-dioxan-2-yl)methanol (66 mg, 0.21 mmol). The vial was placed under N₂ and THF (1.0 mL) was added, followed by sodium hydride (60% in mineral oil, 12.5 mg, 0.31 mmol). The reaction was stirred for 15 mins before addition of iodomethane (65 μL, 1.04 mmol) and the reaction was then stirred at for 2 h. The reaction was quenched by addition of MeOH and filtered through a plug of SiO₂ with Et₂O. The eluent was concentrated in vacuo to give 5-((4-bromophenoxy)methyl)-3-(methoxymethyl)-2-methyl-1,4-dioxane which was used without further purification. LCMS t=0.94, 0.95 min, m/z=331.2, 333.2 [M+H].

### Examples 393: 5-(4-((6-(methoxymethyl)-5-methyl-1,4-dioxan-2-yl)methoxy)phenyl)-2-oxo-6-(trifluoromethyl)-1,2-dihydropyridine-3-carboxamide (Peak 2) (393)

5-(4-((6-methoxy-5-methyl-1,4-dioxan-2-yl)methoxy)phenyl)-2-oxo-6-(trifluoromethyl)-1,2-dihydropyridine-3-carboxamide (peak 2) (393) was obtained by reverse phase HPLC purification of the product of Example 392 as a racemic single diastereomer of undefined stereochemistry. Peak 2: ¹H NMR (500 MHz, DMSO-d6) δ 13.64 (br. s, 1H), 8.46 (br. s, 1H), 8.28 (s, 1H), 8.17 (br. s, 1H), 7.26 (d, J=8.2 Hz, 2H), 7.03 (d, J=8.2 Hz, 2H), 4.08-3.94 (m, 3H), 3.92-3.86 (m, 1H), 3.81-3.74 (m, 2H), 3.71 (dd, J=10.5, 6.8 Hz, 1H), 3.57 (dd, J=10.5, 4.6 Hz, 1H), 3.48 (t, J=10.6 Hz, 1H), 3.29 (s, 3H), 1.04 (d, J=6.3 Hz, 3H).

### Example 394: 5-(4-(((1S,3S,6R)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide (394)

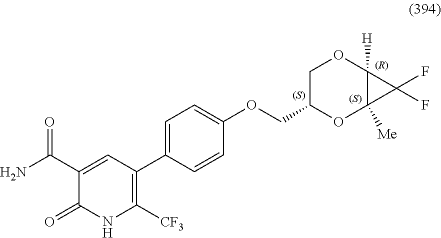

5-(4-(((1S,3S,6R)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide (394) was obtained using step 1 in the synthesis of 5-(4-((6-(hydroxymethyl)-1,4-dioxan-2-yl)methoxy)phenyl)-2-oxo-6-(trifluoromethyl)-1,2-dihydropyridine-3-carboxamide (384), except (4-fluorophenyl)boronic acid was replaced with (1S,3S,6R)-3-((4-bromophenoxy)methyl)-7,7-difluoro-1-methyl-2,5-dioxabicyclo[4.1.0]heptane. LCMS: t=0.87, m/z=461.3 [M+H]. ¹H NMR (500 MHz, DMSO-d6) δ 13.62 (br. s, 1H), 8.44 (br. s, 1H), 8.26 (s, 1H), 8.16 (br. s, 1H), 7.27 (d, J=8.7 Hz, 2H), 7.05 (d, J=8.7 Hz, 2H), 4.09 (d, J=2.3 Hz, 3H), 3.91-3.84 (m, 2H), 3.59-3.51 (m, 1H), 1.37 (t, J=2.2 Hz, 3H).

### (1S,3S,6R)-3-((4-bromophenoxy)methyl)-7,7-difluoro-1-methyl-2,5-dioxabicyclo[4.1.0]heptane and (1R,3S,6S)-3-((4-bromophenoxy)methyl)-7,7-difluoro-1-methyl-2,5-dioxabicyclo[4.1.0]heptane were Obtained Using the Following Procedure

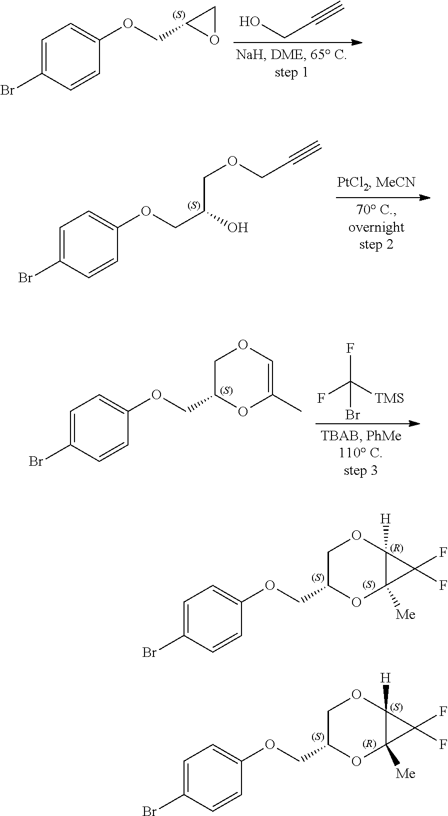

Step 1: To a 40 mL oven dried vial with a stirbar was added sodium hydride (60% in oil, 655 mg, 16.7 mmol) and the vial sealed with a septa top cap and purged with vac/N₂ (×3). DME (24.3 mL) was added, followed by dropwise addition of propargyl alcohol (2.60 mL, 43.7 mmol. The reaction was stirred for 10 mins before the addition of (S)-2-((4-bromophenoxy)methyl)oxirane (2500 mg, 10.9 mmol. The reaction was then heated to 65° C. and stirred for 2.5 h. The reaction was quenched with MeOH and filtered through a plug of SiO₂ with EA. Following concentration in vacuo, mineral oil was removed by filtering through a plug of SiO₂ with heptane and the product was eluted with EA. The eluent was concentrated to give (S)-1-(4-bromophenoxy)-3-(prop-2-yn-1-yloxy)propan-2-ol. LCMS t=0.80, m/z=285.2, 287.2 [M+H].

Step 2: To an oven dried 40 mL vial was added platinum(II) chloride (0.070 g, 0.26 mmol) and (S)-1-(4-bromophenoxy)-3-(prop-2-yn-1-yloxy)propan-2-ol (1.50 g, 5.26 mmol) the reaction was sealed with a septa-top cap and purged with vac/N₂ (×3). Acetonitrile (26.3 mL) was added and the suspension stirred at 70° C. for 5 h. An additional portion of platinum(II) chloride (0.070 g, 0.26 mmol) was added and the reaction was stirred at 70° C. overnight. The reaction was concentrated in vacuo and filtered through a plug of SiO₂ with 15% EA/heptane. Concentration in vacuo gave (S)-2-((4-bromophenoxy)methyl)-6-methyl-2,3-dihydro-1,4-dioxine. LCMS t=1.01, m/z=285.2, 287.2 [M+H].

Step 3: To an oven-dried 20 ml MW vial with a stirbar was added (S)-2-((4-bromophenoxy)methyl)-6-methyl-2,3-dihydro-1,4-dioxine (700 mg, 2.45 mmol) and TBAB (23.7 mg, 0.074 mmol. The vial was sealed and purged with vac/N₂ (×3). Toluene (12.3 ml) and (bromodifluoromethyl)trimethylsilane (1.14 ml, 7.36 mmol) were added and the reaction heated to 110° C. and stirred for 5 h. LCMS shows the formation of the desired mass t=1.03, m/z=335.2, 337.2 [M+H], with some SM remaining. An additional equivalent of carbene reagent was added and the reaction heated to 110° C. for 3 h. After cooling, the reaction was concentrated in vacuo and purified by silica gel chromatography (ISCO, 0-30% EA/Hept) to give two products: (1S,3S,6R)-3-((4-bromophenoxy)methyl)-7,7-difluoro-1-methyl-2,5-dioxabicyclo[4.1.0]heptane and (1R,3S,6S)-3-((4-bromophenoxy)methyl)-7,7-difluoro-1-methyl-2,5-dioxabicyclo[4.1.0]heptane.

### Example 395: 55-(4-(((1R,3S,6S)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide (394)

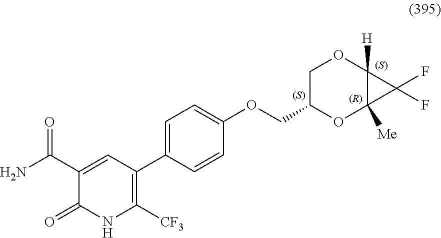

55-(4-(((1R,3S,6S)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide (395) was obtained using the method described in Example 394, except (1S,3S,6R)-3-((4-bromophenoxy)methyl)-7,7-difluoro-1-methyl-2,5-dioxabicyclo[4.1.0]heptane was replaced with (1R,3S,6S)-3-((4-bromophenoxy)methyl)-7,7-difluoro-1-methyl-2,5-dioxabicyclo[4.1.0]heptane. LCMS: t=0.86, m/z=461.3 [M+H]. ¹H NMR (500 MHz, DMSO-d6) δ 13.62 (br. s, 1H), 8.46 (br. s, 1H), 8.26 (s, 1H), 8.15 (br. s, 1H), 7.27 (d, J=8.6 Hz, 2H), 7.03 (d, J=8.6 Hz, 2H), 4.11-4.02 (m, 3H), 3.85-3.78 (m, 2H), 3.58 (ddd, J=10.7, 8.0, 2.3 Hz, 2H), 1.39 (t, J=2.2 Hz, 3H).

### Example 396: 5-(4-(((1R,3R,6S)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide (396)

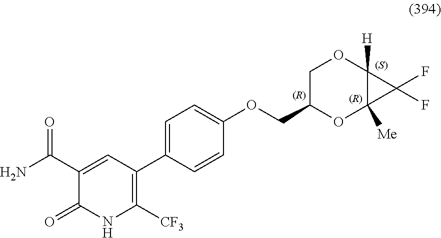

5-(4-(((1R,3R,6S)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide (396) was obtained using the method described in Example 394, except (1S,3S,6R)-3-((4-bromophenoxy)methyl)-7,7-difluoro-1-methyl-2,5-dioxabicyclo[4.1.0]heptane was replaced with (1R,3R,6S)-3-((4-bromophenoxy)methyl)-7,7-difluoro-1-methyl-2,5-dioxabicyclo[4.1.0]heptane. LCMS: t=0.87, m/z=461.3 [M+H]. ¹H NMR (500 MHz, DMSO-d6) δ 13.62 (br. s, 1H), 8.44 (br. s, 1H), 8.26 (s, 1H), 8.16 (br. s, 1H), 7.27 (d, J=8.7 Hz, 2H), 7.05 (d, J=8.7 Hz, 2H), 4.09 (d, J=2.3 Hz, 3H), 3.91-3.84 (m, 2H), 3.59-3.51 (m, 1H), 1.37 (t, J=2.2 Hz, 3H).

(1R,3R,6S)-3-((4-bromophenoxy)methyl)-7,7-difluoro-1-methyl-2,5-dioxabicyclo[4.1.0]heptane was obtained using the method described for the synthesis of (1S,3S,6R)-3-((4-bromophenoxy)methyl)-7,7-difluoro-1-methyl-2,5-dioxabicyclo[4.1.0]heptane except (S)-2-((4-bromophenoxy)methyl)oxirane was replaced with. (R)-2-((4-bromophenoxy)methyl)oxirane.

### Example 397: 5-(4-(((1S,3R,6R)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide (397)

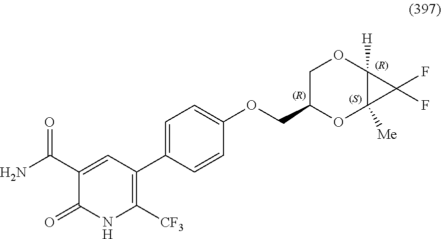

5-(4-(((1S,3R,6R)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide (397) was obtained using the method described in Example 395, except (1R,3S,6S)-3-((4-bromophenoxy)methyl)-7,7-difluoro-1-methyl-2,5-dioxabicyclo[4.1.0]heptane was replaced with (1S,3R,6R)-3-((4-bromophenoxy)methyl)-7,7-difluoro-1-methyl-2,5-dioxabicyclo[4.1.0]heptane. LCMS: t=0.86, m/z=461.3 [M+H]. ¹H NMR (500 MHz, DMSO-d6) δ 13.62 (br. s, 1H), 8.46 (br. s, 1H), 8.26 (s, 1H), 8.15 (br. s, 1H), 7.27 (d, J=8.6 Hz, 2H), 7.03 (d, J=8.6 Hz, 2H), 4.11-4.02 (m, 3H), 3.85-3.78 (m, 2H), 3.58 (ddd, J=10.7, 8.0, 2.3 Hz, 2H), 1.39 (t, J=2.2 Hz, 3H).

(1S,3R,6R)-3-((4-bromophenoxy)methyl)-7,7-difluoro-1-methyl-2,5-dioxabicyclo[4.1.0]heptane was obtained using the method described for the synthesis of (1R,3S,6S)-3-((4-bromophenoxy)methyl)-7,7-difluoro-1-methyl-2,5-dioxabicyclo[4.1.0]heptane, except (S)-2-((4-bromophenoxy)methyl)oxirane was replaced with. (R)-2-((4-bromophenoxy)methyl)oxirane.

### Example 398: 5-(4-(((2S,6S)-6-(methoxymethyl)-1,4-dioxan-2-yl)methoxy)phenyl)-2-oxo-6-(trifluoromethyl)-1,2-dihydropyridine-3-carboxamide (398)

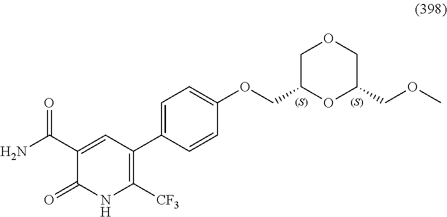

5-(4-(((2S,6S)-6-(methoxymethyl)-1,4-dioxan-2-yl)methoxy)phenyl)-2-oxo-6-(trifluoromethyl)-1,2-dihydropyridine-3-carboxamide (398) was obtained by RP-HPLC from the product obtained using the procedure described for Example 348, except 2-((4-bromophenoxy)methyl)-6-(difluoromethyl)-1,4-dioxane was replaced with a mixture of (2S,6R)-2-((4-bromophenoxy)methyl)-6-(methoxymethyl)-1,4-dioxane and (2S,6S)-2-((4-bromophenoxy)methyl)-6-(methoxymethyl)-1,4-dioxane. ¹H NMR (500 MHz, DMSO-d₆) δ 13.65 (s, 1H), 8.48 (s, 1H), 8.29 (s, 1H), 8.19 (s, 1H), 7.29 (d, J=8.6 Hz, 2H), 7.05 (d, J=8.7 Hz, 2H), 4.07-4.00 (m, 2H), 4.00-3.94 (m, 1H), 3.87 (dd, J=11.3, 2.6 Hz, 1H), 3.81 (dtd, J=10.2, 4.9, 2.6 Hz, 1H), 3.76 (dd, J=11.4, 2.7 Hz, 1H), 3.39 (dd, J=10.7, 5.4 Hz, 1H), 3.35 (s, 1H), 3.33-3.31 (m, 1H), 3.30-3.24 (m, 4H). LCMS Rt=2.78 min, m/z=443.2 (M+H).

### (2S,6S)-2-((4-bromophenoxy)methyl)-6-(methoxymethyl)-1,4-dioxane and (2S,6R)-2-((4-bromophenoxy)methyl)-6-(methoxymethyl)-1,4-dioxane were Obtained Using the Following Procedure

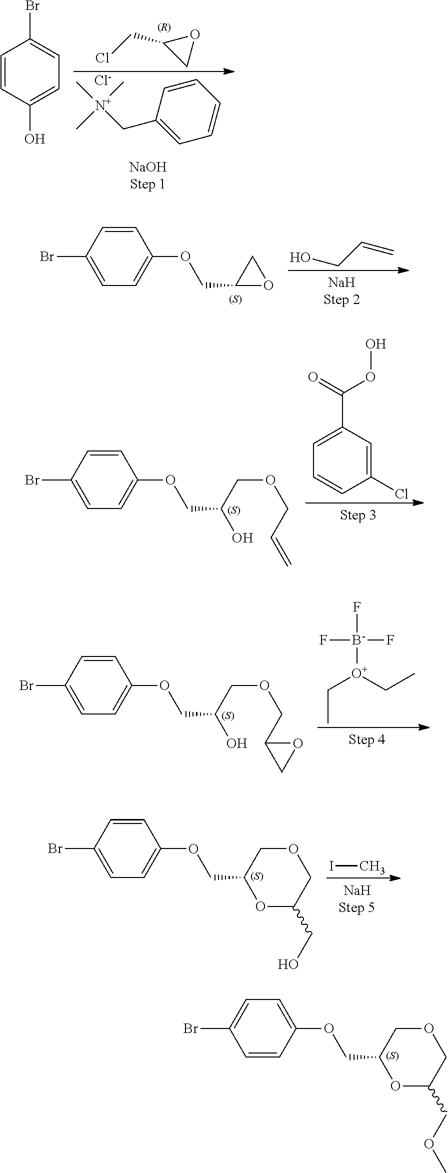

Step 1: To a 500 mL 3-necked flask was added (S)-epichlorohydrin (58.3 g, 630 mmol) and water (10 mL) and the mixture cooled to −5° C. and stirred vigorously. To a separate 250 mL flask was added 4-bromophenol (72.7 g, 420 mmol), benzyltrimethylammonium chloride (1.560 g, 8.40 mmol) and sodium hydroxide (16.80 g, 420 mmol) and water was added and the reaction mixture stirred until homogenous. This mixture was added over 20 mins to the cooled reaction mixture containing flask (S)-epichlorohydrin. The resulting mixture was stirred for 5.5 h at −5° C., warmed to rt and stirred overnight The reaction mixture was diluted with water and extracted into Et2O (×4). The organic layers were combined, washed with brine, dried over Na₂SO₄, filtered through a plug of SiO₂, concentrated and the residual oil was crystallised in the freezer from MeOH/H₂O (˜10%) to give the crystalline hydrate which decomposed to a biphasic liquid upon sitting, removal of the aqueous layer and azeotroping with benzene×2 followed by chilling in the freezer gave (S)-2-((4-bromophenoxy)methyl)oxirane.

Step 2: To a 500 mL flask was added dry DMF (170 ml) and allyl alcohol (29.7 mL, 437 mmol). The flask was cooled to 0° C. and put under a stream of N2, sodium hydride (3.4 g, 135 mmol) was added (2×1.7 g portions). The mixture was stirred at 0° C. until a homogeneous solution had formed (15 min), after which (S)-2-((4-bromophenoxy)methyl)oxirane (20 g, 87 mmol) was added. The mixture was then heated to 40° C. for 1 h. the mixture was quenched with saturated aqueous NH₄Cl and stirred until gas evolution ceased. The mixture was diluted with (1 L) and extracted with EtOAc (2×200 mL). The organic layers were combined, washed with water and brine, dried over Na₂SO₄, filtered and concentrated to give (S)-1-(allyloxy)-3-(4-bromophenoxy)propan-2-ol which was used without further purification.

Step 3: To a 1 L flask was added (S)-1-(allyloxy)-3-(4-bromophenoxy)propan-2-ol (24.81 g, 86 mmol), dry DCM (200 ml) and 3-chloroperoxybenzoic acid (25.2 g, 112 mmol) and the reaction mixture stirred at rt for 17 h. The mixture was quenched with 10% (w/v) aqueous Na₂SO₃, stirred for 1 h and extracted with DCM. The organic layer was washed with saturated aq. NaHCO₃ twice, dried over sodium sulfate and concentrated to give (2S)-1-(4-bromophenoxy)-3-(oxiran-2-ylmethoxy)propan-2-ol which was used directly without further purification. LCMS (t=0.66 min, m/z=303.2 [M+H]+).

Step 4: To a 2 L flask purged with N₂ was added (2S)-1-(4-bromophenoxy)-3-(oxiran-2-ylmethoxy)propan-2-ol (24.88 g, 82 mmol) and dry DCM (1320 mL). Under a stream of N₂, boron trifluoride etherate (2.080 mL, 16.41 mmol) was added, and the reaction stirred at rt under N₂ for 2 h. The mixture was quenched with saturated aq. NaHCO₃, stirred at rt for 10 min and extracted with DCM. The organic layers were combined, dried over sodium sulfate, and concentrated to give crude product. The crude product which was purified by CombiFlash (GOLD column running a gradient of 0 to 60% EtOAc/heptane over 5 minutes) to give diastereomers (1:1) ((2R,6S)-6-((4-bromophenoxy)methyl)-1,4-dioxan-2-yl)methanol and ((2S,6S)-6-((4-bromophenoxy)methyl)-1,4-dioxan-2-yl)methanol.

Step 5: To a 5 mL microwave vial was added (6S)-6-((4-bromophenoxy)methyl)-1,4-dioxan-2-yl)methanol (250 mg, 0.825 mmol), dry DMF (3 mL), and sodium hydride (62.5 mg, 2.474 mmol). The mixture was stirred at rt for 15 min, then iodomethane (0.16 mL, 2.56 mmol) was added, and the reaction continued at rt for 1 h. The reaction was quenched with aqueous NH₄Cl, diluted with EtOAc and extracted with EtOAc twice. The organic layers were combined, washed with water and brine, dried over sodium sulfate, filtered and concentrated to give (2S,6R)-2-((4-bromophenoxy)methyl)-6-(methoxymethyl)-1,4-dioxane and (2S,6S)-2-((4-bromophenoxy)methyl)-6-(methoxymethyl)-1,4-dioxane.

### Example 399: 5-(4-(((2S,6R)-6-(methoxymethyl)-1,4-dioxan-2-yl)methoxy)phenyl)-2-oxo-6-(trifluoromethyl)-1,2-dihydropyridine-3-carboxamide (399)

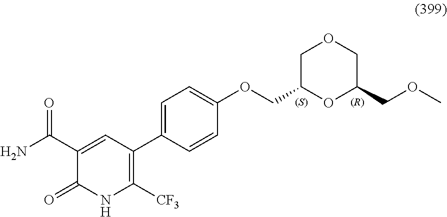

5-(4-(((2S,6R)-6-(methoxymethyl)-1,4-dioxan-2-yl)methoxy)phenyl)-2-oxo-6-(trifluoromethyl)-1,2-dihydropyridine-3-carboxamide (399) was obtained by RP-HPLC of the mixture obtained in Example 398. ¹H NMR (500 MHz, DMSO-d₆) δ 13.68 (s, 1H), 8.48 (s, 1H), 8.29 (s, 1H), 8.19 (s, 1H), 7.29 (d, J=8.6 Hz, 2H), 7.07 (d, J=8.8 Hz, 2H), 4.22 (dd, J=10.2, 6.3 Hz, 1H), 4.17 (dd, J=10.2, 5.5 Hz, 1H), 4.08 (qd, J=5.5, 3.3 Hz, 1H), 3.95 (qd, J=5.9, 3.2 Hz, 1H), 3.75 (dd, J=11.7, 3.3 Hz, 1H), 3.71 (dd, J=11.6, 3.2 Hz, 1H), 3.64 (dd, J=11.7, 5.0 Hz, 1H), 3.53-3.42 (m, 3H), 3.29 (s, 3H). LCMS Rt=2.69 min, m/z=443.1 (M+H).

### Example 400: 5-(4-(((2R,6R)-6-(methoxymethyl)-1,4-dioxan-2-yl)methoxy)phenyl)-2-oxo-6-(trifluoromethyl)-1,2-dihydropyridine-3-carboxamide (Cis, SFC Peak 2) (400)

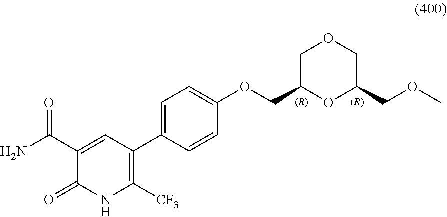

T5-(4-(((2R,6R)-6-(methoxymethyl)-1,4-dioxan-2-yl)methoxy)phenyl)-2-oxo-6-(trifluoromethyl)-1,2-dihydropyridine-3-carboxamide (cis, SFC peak 2) was made from the same procedure used to make example 397, except the mixture of (2S,6R)-2-((4-bromophenoxy)methyl)-6-(methoxymethyl)-1,4-dioxane and (2S,6S)-2-((4-bromophenoxy)methyl)-6-(methoxymethyl)-1,4-dioxane was replaced with a mixture of (2R,6R)-2-((4-bromophenoxy)methyl)-6-(methoxymethyl)-1,4-dioxane and (2R,6S)-2-((4-bromophenoxy)methyl)-6-(methoxymethyl)-1,4-dioxane. ¹H NMR (500 MHz, DMSO-d₆) δ=13.63 (s, 1H), 8.46 (s, 1H), 8.27 (s, 1H), 8.22-8.10 (m, 1H), 7.26 (d, J=8.0 Hz, 2H), 7.02 (d, J=8.0 Hz, 2H), 4.05-3.91 (m, 3H), 3.87-3.70 (m, 3H), 3.33-3.25 (m, 4H), 3.24 (s, 3H). LCMS Rt=0.72 min, m/z=443.2 (M+H). Chiral HPLC: Rt=3.75 min, ee value=100%.

(2R,6R)-2-((4-bromophenoxy)methyl)-6-(methoxymethyl)-1,4-dioxane and (2R,6S)-2-((4-bromophenoxy)methyl)-6-(methoxymethyl)-1,4-dioxane were obtained as described for the preparation of (2S,6S)-2-((4-bromophenoxy)methy-6-(methoxymethyl)-1,4-dioxane and (2S,6R)-2-((4-bromophenoxy)methy-6-(methoxymethyl)-1,4-dioxane, except (S)-epichlorohydrin was replaced with (R)-epichlorohydrin.

### Example 401: 5-(4-(((2R,6S)-6-(methoxymethyl)-1,4-dioxan-2-yl)methoxy)phenyl)-2-oxo-6-(trifluoromethyl)-1,2-dihydropyridine-3-carboxamide (Trans, SFC Peak 1) (401)

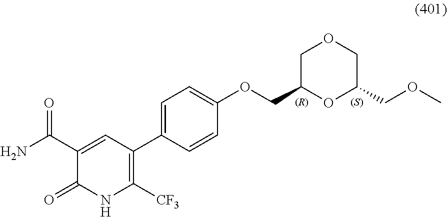

5-(4-(((2R,6S)-6-(methoxymethyl)-1,4-dioxan-2-yl)methoxy)phenyl)-2-oxo-6-(trifluoromethyl)-1,2-dihydropyridine-3-carboxamide (trans, SFC peak 1) (401) was obtained by chiral SFC of the mixture from Example 400. ¹H NMR (500 MHz, DMSO-d₆) δ=8.71-8.49 (s, 1H), 8.25 (s, 1H), 8.19-8.06 (m, 1H), 7.26 (d, J=8.0 Hz, 2H), 7.05 (d, J=8.0 Hz, 2H), 4.23-4.12 (m, 2H), 4.09-4.03 (m, 1H), 3.97-3.90 (m, 1H), 3.76-3.71 (m, 1H), 3.71-3.66 (m, 1H), 3.65-3.60 (m, 1H), 3.48-3.46 (m, 1H), 3.48-3.47 (m, 1H), 3.45-3.43 (m, 2H), 3.27 (s, 3H). LCMS Rt=0.70 min, m/z=443.3 (M+H). Chiral HPLC: Rt=3.37 min, ee value=100%.

### Example 402: 5-(4-(((2S,6R)-6-(isopropoxymethyl)-1,4-dioxan-2-yl)methoxy)phenyl)-2-oxo-6-(trifluoromethyl)-1,2-dihydropyridine-3-carboxamide (402)

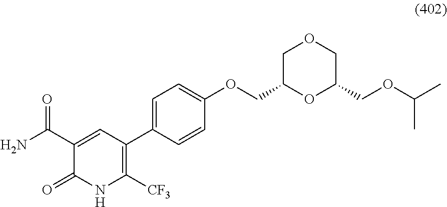 [SMILES: CC(C)OC[C@H]1COC[C@@H](COc2ccc(-c3cc(C(N)=O)c(=O)[nH]c3C(F)(F)F)cc2)O1]

5-(4-(((2S,6R)-6-(isopropoxymethyl)-1,4-dioxan-2-yl)methoxy)phenyl)-2-oxo-6-(trifluoromethyl)-1,2-dihydropyridine-3-carboxamide (402) was obtained using the procedure described for Example 348, except 2-((4-bromophenoxy)methyl)-6-(difluoromethyl)-1,4-dioxane was replaced with (2S)-2-((4-bromophenoxy)methyl)-6-(isopropoxymethyl)-1,4-dioxane. ¹H NMR (500 MHz, DMSO-d₆) δ 13.65 (s, 1H), 8.47 (s, 1H), 8.29 (s, 1H), 8.19 (s, 1H), 7.28 (d, J=8.7 Hz, 2H), 7.05 (d, J=8.8 Hz, 2H), 4.07-3.94 (m, 3H), 3.87 (dd, J=11.3, 2.6 Hz, 1H), 3.76 (tt, J=7.9, 2.6 Hz, 2H), 3.55 (hept, J=6.1 Hz, 1H), 3.42 (dd, J=10.4, 5.1 Hz, 1H), 3.33 (d, J=5.3 Hz, 2H), 3.26 (t, J=11.4 Hz, 1H), 1.09 (d, J=6.1 Hz, 6H). LCMS Rt=3.47 min, m/z=471.1 (M+H).

### (2S)-2-((4-bromophenoxy)methyl)-6-(isopropoxymethyl)-1,4-dioxane was Obtained Using the Following Procedure

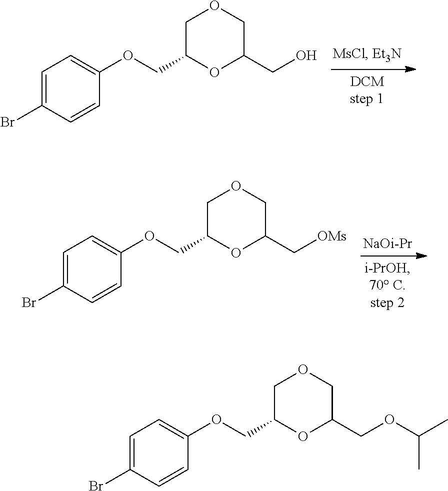 [SMILES: OCC1COC[C@@H](COc2ccc(Br)cc2)O1 ; CS(=O)(=O)OCC1COC[C@@H](COc2ccc(Br)cc2)O1 ; CC(C)OCC1COC[C@@H](COc2ccc(Br)cc2)O1 ; "NaOi-Pr"]

Step 1: To a stirring solution of ((6S)-6-((4-bromophenoxy)methyl)-1,4-dioxan-2-yl)methanol (1 g, 3.30 mmol) in dry DCM (20 mL) was added triethylamine (0.690 mL, 4.95 mmol, followed by dropwise addition of methanesulfonyl chloride (0.283 mL, 3.63 mmol. The reaction was then continued at RT for 30 min. The crude was purified via silica gel chromatography (ISCO, 0 to 100% ethyl acetate/heptane) to give ((6S)-6-((4-bromophenoxy)methyl)-1,4-dioxan-2-yl)methyl methanesulfonate. LCMS Rt=0.78 min, m/z=381.1, 383.2 (M+H).

Step 2: To a 20 ml microwave vial was added ((6S)-6-((4-bromophenoxy)methyl)-1,4-dioxan-2-yl)methyl methanesulfonate (400 mg, 1.049 mmol) and dry 2-propanol (10 mL). The mixture was sonicated to dissolve as much of the mesylate as possible, then sodium isopropoxide (517 mg, 6.30 mmol) was added. The suspension was heated to 70° C. with vigorous stirring for 8 hours. The crude was filtered, concentrated down, and purified via silica gel chromatography (ISCO, 0 to 30% ethyl acetate/heptane) to give (2S)-2-((4-bromophenoxy)methyl)-6-(isopropoxymethyl)-1,4-dioxane. LCMS Rt=0.90 min, m/z=343.4, 345.3 (M+H).

### Example 403: 5-(4-(((2S,6S)-6-(isopropoxymethyl)-1,4-dioxan-2-yl)methoxy)phenyl)-2-oxo-6-(trifluoromethyl)-1,2-dihydropyridine-3-carboxamide (403)

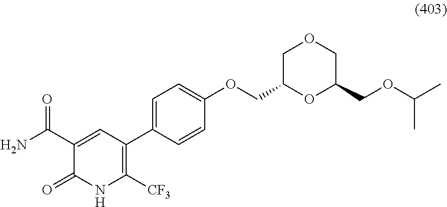 [SMILES: CC(C)OC[C@@H]1COC[C@@H](COc2ccc(-c3cc(C(N)=O)c(=O)[nH]c3C(F)(F)F)cc2)O1]

5-(4-(((2S,6S)-6-(isopropoxymethyl)-1,4-dioxan-2-yl)methoxy)phenyl)-2-oxo-6-(trifluoromethyl)-1,2-dihydropyridine-3-carboxamide (403) by RP-HPLC of the product from Example 402. ¹H NMR (500 MHz, DMSO-d₆) δ 13.65 (s, 1H), 8.48 (s, 1H), 8.29 (s, 1H), 8.18 (s, 1H), 7.29 (d, J=8.6 Hz, 2H), 7.07 (d, J=8.7 Hz, 2H), 4.19 (qd, J=10.3, 6.0 Hz, 2H), 4.10-4.05 (m, 1H), 3.88 (ddt, J=9.3, 6.0, 3.2 Hz, 1H), 3.76 (dd, J=11.7, 3.2 Hz, 1H), 3.71 (dd, J=11.6, 3.2 Hz, 1H), 3.63 (dd, J=11.7, 5.2 Hz, 1H), 3.62-3.52 (m, 1H), 3.54-3.47 (m, 3H), 1.10 (d, J=6.1 Hz, 6H). LCMS Rt=3.39 min, m/z=471.1 (M+H).

### Example 404: 5-(4-(((2S,6S)-6-cyclopropyl-1,4-dioxan-2-yl)methoxy)phenyl)-2-oxo-6-(trifluoromethyl)-1,2-dihydropyridine-3-carboxamide (404)

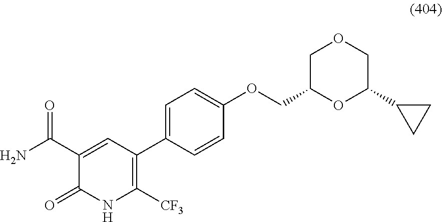 [SMILES: NC(=O)c1cc(-c2ccc(OC[C@@H]3COC[C@H](C4CC4)O3)cc2)c(C(F)(F)F)[nH]c1=O]

5-(4-(((2S,6S)-6-cyclopropyl-1,4-dioxan-2-yl)methoxy)phenyl)-2-oxo-6-(trifluoromethyl)-1,2-dihydropyridine-3-carboxamide (404) was obtained using the procedure described for Example 348, except 2-((4-bromophenoxy)methyl)-6-(difluoromethyl)-1,4-dioxane was replaced with (2S,6S)-2-((4-bromophenoxy)methyl)-6-cyclopropyl-1,4-dioxane. ¹H NMR (500 MHz, DMSO-d₆) δ 13.65 (s, 1H), 8.48 (s, 1H), 8.29 (s, 1H), 8.19 (s, 1H), 7.38-7.19 (m, 2H), 7.11-6.97 (m, 2H), 4.05 (dd, J=10.5, 5.4 Hz, 1H), 4.00 (dd, J=10.4, 4.4 Hz, 1H), 3.89 (ddd, J=10.4, 4.9, 2.8 Hz, 1H), 3.87-3.83 (m, 1H), 3.78 (dd, J=11.4, 2.6 Hz, 1H), 3.32 (d, J=9.8 Hz, 1H), 3.27 (d, J=11.0 Hz, 1H), 2.97 (ddd, J=10.5, 8.2, 2.6 Hz, 1H), 0.76 (qt, J=8.3, 4.9 Hz, 1H), 0.50-0.40 (m, 2H), 0.33 (dtd, J=9.0, 4.6, 2.8 Hz, 1H), 0.30-0.24 (m, 1H). LCMS Rt=0.75 min, m/z=439.3 (M+H).

### (2S,6S)-2-((4-bromophenoxy)methyl)-6-cyclopropyl-1,4-dioxane was Obtained Using the Following Procedure

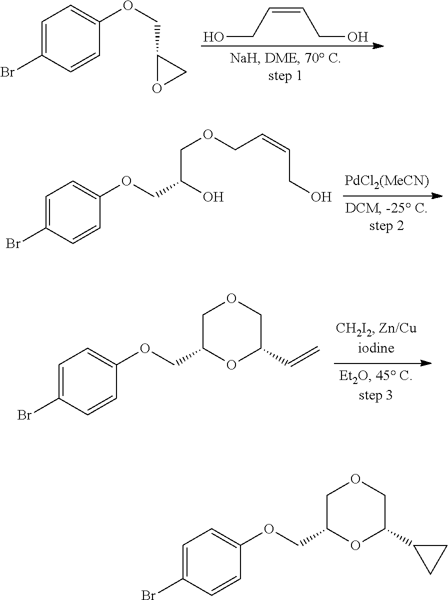 [SMILES: Brc1ccc(OC[C@@H]2CO2)cc1 ; OC/C=C\CO ; OC/C=C\COC[C@H](O)COc1ccc(Br)cc1 ; C=C[C@H]1COC[C@@H](COc2ccc(Br)cc2)O1 ; Brc1ccc(OC[C@@H]2COC[C@H](C3CC3)O2)cc1]

Step 1: To a 500 mL flask was added NaH (1.66 g, 65.7 mmol) and dry DME (140 mL). Then (Z)-but-2-ene-1,4-diol (18 ml, 219 mmol) was added dropwise, and the mixture stirred at RT for 10 min. Then (S)-2-((4-bromophenoxy)methyl)oxirane (10 g, 43.7 mmol) was added, and the mixture heated to 70° C. for 2 hours. The reaction was quenched with sat. NH₄Cl, concentrated, diluted with EtOAc, washed with water twice, then dried over sodium sulfate and concentrated. The crude product was purified via silica gel chromatography twice (ISCO, 0 to 100% ethyl acetate/heptane) to give (S,Z)-4-(3-(4-bromophenoxy)-2-hydroxypropoxy)but-2-en-1-ol. LCMS Rt=0.64 min, m/z=317.2, 319.2 (M+H).

Step 2: To a 500 mL flask was added (S,Z)-4-(3-(4-bromophenoxy)-2-hydroxypropoxy)but-2-en-1-ol (10.9 g, 34.5 mmol) and dry DCM (200 mL). The solution was cooled to −25° C. by means of an immersion chiller, and PdCl₂(MeCN) (0.895 g, 3.45 mmol) was added. The reaction was then stirred at −25° C. for 88 hours. It was quenched with 1M aq. HCl, extracted with DCM, dried over sodium sulfate, and concentrated. The crude was then purified via silica gel chromatography four times (ISCO, 0 to 20% ethyl acetate/heptane) to separate diastereomers and give (2S,6S)-2-((4-bromophenoxy)methyl)-6-vinyl-1,4-dioxane. ¹H NMR (500 MHz, CDCl₃) δ 7.39 (d, J=9.0 Hz, 2H), 6.81 (d, J=9.0 Hz, 2H), 5.77 (ddd, J=17.1, 10.8, 5.9 Hz, 1H), 5.39 (dt, J=17.4, 1.5 Hz, 1H), 5.27 (dt, J=10.7, 1.4 Hz, 1H), 4.22 (dddt, J=10.2, 5.7, 2.8, 1.3 Hz, 1H), 4.11-4.03 (m, 2H), 3.96 (dd, J=11.4, 2.5 Hz, 1H), 3.91 (dd, J=9.4, 5.4 Hz, 1H), 3.79 (dd, J=11.5, 2.8 Hz, 1H), 3.45 (dd, J=11.4, 10.1 Hz, 1H), 3.31 (dd, J=11.6, 10.4 Hz, 1H).

Step 3: To a 250 mL flask was added give (2S,6S)-2-((4-bromophenoxy)methyl)-6-vinyl-1,4-dioxane (4.12 g, 13.8 mmol) and dry Et₂O (70 mL). Then zinc-copper couple (14.41 g, 20 mmol) was added, followed by iodine (10.49 g, 41.3 mmol) and the reaction heated to 45° C. The reaction was stirred until the brown color of iodine had been discharged then diiodomethane (2.2 mL, 27.3 mmol was added, and the reaction continued at 45° C. for 2 hours. Additional diiodomethane (7 mL, 87 mmol) was then added via syringe pump over 6 hours, and the reaction stirred at 45° C. for 19 hours. More zinc-copper couple (3.60 g, 55.1 mmol) was added, and additional diiodomethane (7 mL, 87 mmol) was added via syringe pump over 6 hours. The reaction was stirred at 45° C. for another hour, then it was filtered, and quenched with 1M HCl. The crude was extracted with ether, washed with 10% (w/v) aqueous sodium sulfite, dried over sodium sulfate, and concentrated. The crude was then purified via silica gel chromatography twice (ISCO, 0 to 20% ethyl acetate/heptane) to give (2S,6S)-2-((4-bromophenoxy)methyl)-6-cyclopropyl-1,4-dioxane. LCMS Rt=0.88 min, m/z=313.3, 315.2 (M+H).

### Example 405: 5-(4-(((2S,6S)-6-(methoxymethyl)-6-methyl-1,4-dioxan-2-yl)methoxy)phenyl)-2-oxo-6-(trifluoromethyl)-1,2-dihydropyridine-3-carboxamide (405)

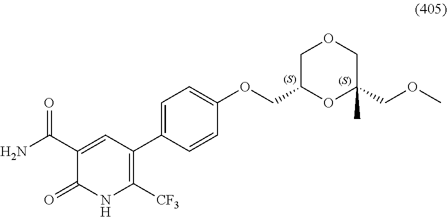

5-(4-(((2S,6S)-6-(methoxymethyl)-6-methyl-1,4-dioxan-2-yl)methoxy)phenyl)-2-oxo-6-(trifluoromethyl)-1,2-dihydropyridine-3-carboxamide (405) was obtained using the method described in steps 8-9 for the preparation of 5-(4-((3-cyclopropyl-6,7-dihydro-4H-[1,2,3]triazolo[5,1-c][1,4]oxazin-6-yl)methoxy)phenyl)-2-oxo-6-(trifluoromethyl)-1,2-dihydropyridine-3-carboxamide (380), except 6-((4-bromophenoxy)methyl)-3-cyclopropyl-6,7-dihydro-4H-[1,2,3]triazolo[5,1-c][1,4]oxazine was replaced with (2S,6S)-6-((4-bromophenoxy)methyl)-2-(methoxymethyl)-2-methyl-1,4-dioxane. LCMS t=0.88 min, m/z=457.5 [M+H]. ¹H NMR (500 MHz, DMSO-d6) δ 13.63 (br. s, 1H), 8.46 (br. s, 1H), 8.27 (s, 1H), 8.17 (br. s, 1H), 7.26 (d, J=8.4 Hz, 2H), 7.03 (d, J=8.4 Hz, 2H), 4.21-4.15 (m, 1H), 4.00-3.91 (m, 2H), 3.88 (dd, J=11.2, 2.8 Hz, 1H), 3.51 (d, J=11.2 Hz, 1H), 3.36 (d, J=11.2 Hz, 2H), 3.31-3.24 (m, 5H), 3.22 (s, 2H).

### (2S,6S)-6-((4-bromophenoxy)methyl)-2-(methoxymethyl)-2-methyl-1,4-dioxane and (2R,6S)-6-((4-bromophenoxy)methyl)-2-(methoxymethyl)-2-methyl-1,4-dioxane were Obtained Using the Following Procedure

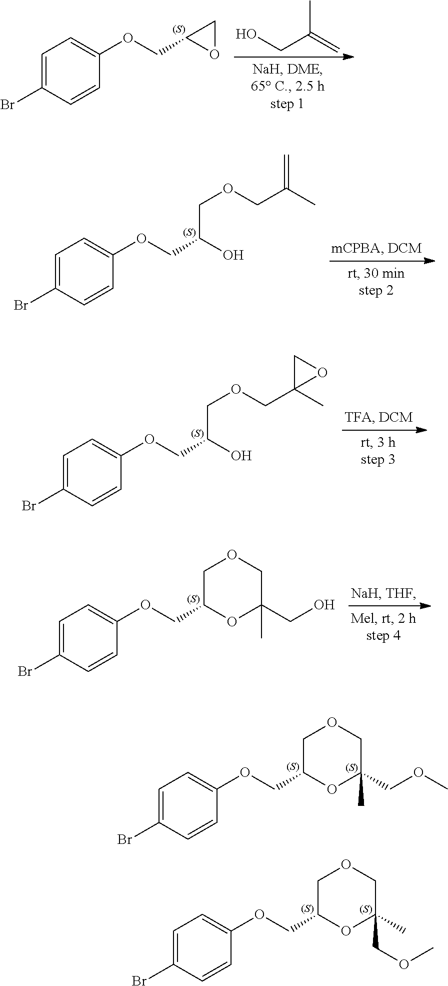

Step 1: To a 40 ml oven-dried vial with a stirbar was added sodium hydride (60% in mineral oil, 655 mg, 16.4 mmol) the vial was sealed with a septa top cap and purged with vac/N₂ (×3). DME (22 mL) was added, followed by dropwise addition of 2-methylprop-2-en-1-ol (3.70 mL, 43.7 mmol) at 0° C. The reaction was stirred for 10 mins before the addition of (S)-2-((4-bromophenoxy)methyl)oxirane (2500 mg, 10.9 mmol). The reaction was then heated to 65° C. and stirred for 2.5 h. The reaction was quenched with MeOH and filtered through a plug of SiO₂ with EA. Following concentration in vacuo the mineral oil was removed by filtering through a plug of SiO₂ with 10% EA/heptane and the product was eluted with Et₂O. Concentration in vacuo gave(S)-1-(4-bromophenoxy)-3-((2-methylallyl)oxy)propan-2-ol. LCMS t=0.92, m/z=301.2, 303.2 [M+H].

Step 2: To a 100 mL RBF with a stirbar was added (S)-1-(4-bromophenoxy)-3-((2-methylallyl)oxy)propan-2-ol (3.0 g, 9.96 mmol), DCM (24.9 ml) and mCPBA (77 w %, 2.90 g, 12.95 mmol) and the mixture stirred at rt for 30 mins. Na₂S₂O_{3(aq)} was added and the reaction mixture stirred vigorously for 5 mins. The reaction mixture was diluted with Et₂O, washed with Na₂S2O₃₈, with NaHCO_{3(aq)} (×2) and brine. The organic layer was dried over Na₂SO₄, filtered through a plug of SiO₂ and concentrated in vacuo to give (2S)-1-(4-bromophenoxy)-3-((2-methyloxiran-2-yl)methoxy)propan-2-ol. LCMS t=0.78 min, m/z=317.2, 319.2 [M+H].

Step 3: To a 100 mL RBF with a stirbar was added (2S)-1-(4-bromophenoxy)-3-((2-methyloxiran-2-yl)methoxy)propan-2-ol (1.80 g, 5.7 mmol), DCM (114 mL) and TFA (0.53 mL, 6.8 mmol) and the reaction stirred at rt for 3 h. L. 20 mL of 2M NaOH_{(aq)} and 20 mL of MeOH were added and the resulting mixture was stirred vigorously for 1 h. The reaction was diluted with brine and extracted with DCM (×3). The combined organic layers were dried over Na₂SO₄, filtered through a plug of SiO₂ and concentrated in vacuo to give ((6S)-6-((4-bromophenoxy)methyl)-2-methyl-1,4-dioxan-2-yl)methanol which was used in the next reaction without further purification. LCMS t=0.77 min, m/z=317.2, 319.2 [M+H] (2× peaks).

Step 4: To a 100 mL RBF with a stirbar was added crude ((6S)-6-((4-bromophenoxy)methyl)-2-methyl-1,4-dioxan-2-yl)methanol (1.40 g, 4.4 mmol) and the flask was purged with N₂ before the addition of THF (22.1 mL), and then sodium hydride (60 w % in mineral oil, 353 mg, 8.8 mmol). The mixture was then stirred for 15 mins before addition of iodomethane (1.38 mL, 22.1 mmol). The mixture was then stirred at rt for 2 h. The reaction mixture was quenched with MeOH and filtered through a plug of SiO₂ with EA and concentrated in vacuo. Purification by silica gel chromatography (ISCO, 0-40% EA/Heptane) followed by separation by chiral chromatography gave two products: (2S,6S)-6-((4-bromophenoxy)methyl)-2-(methoxymethyl)-2-methyl-1,4-dioxane and (2R,6S)-6-((4-bromophenoxy)methyl)-2-(methoxymethyl)-2-methyl-1,4-dioxane. LCMS t=0.95 min, m/z=331.2, 333.2 [M+H].

### Example 406: 5-(4-(((2S,6R)-6-(methoxymethyl)-6-methyl-1,4-dioxan-2-yl)methoxy)phenyl)-2-oxo-6-(trifluoromethyl)-1,2-dihydropyridine-3-carboxamide (406)

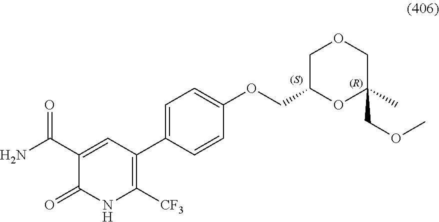

5-(4-(((2S,6R)-6-(methoxymethyl)-6-methyl-1,4-dioxan-2-yl)methoxy)phenyl)-2-oxo-6-(trifluoromethyl)-1,2-dihydropyridine-3-carboxamide (406) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-(methoxymethyl)-2-methyl-1,4-dioxane. LCMS t=0.86 min, m/z=457.5 [M+H]. ¹H NMR (500 MHz, DMSO-d₆) δ 13.6 (br. s, 1H), 8.48 (s, 1H), 8.28 (br. s, 1H), 8.18 (s, 1H), 7.30-7.22 (m, 2H), 7.06-7.00 (m, 2H), 4.18 (dtd, J=10.4, 4.9, 3.0 Hz, 1H), 3.99-3.92 (m, 2H), 3.89 (dd, J=11.3, 3.0 Hz, 1H), 3.67 (d, J=11.3 Hz, 1H), 3.64 (d, J=9.5 Hz, 1H), 3.48 (d, J=9.5 Hz, 1H), 3.32-3.26 (m, 1H), 3.15 (d, J=11.6 Hz, 1H), 1.04 (s, 3H).

### Example 407: 5-(4-(((2R,6R)-6-(methoxymethyl)-6-methyl-1,4-dioxan-2-yl)methoxy)phenyl)-2-oxo-6-(trifluoromethyl)-1,2-dihydropyridine-3-carboxamide (407)

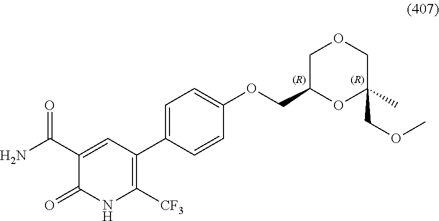

5-(4-(((2R,6R)-6-(methoxymethyl)-6-methyl-1,4-dioxan-2-yl)methoxy)phenyl)-2-oxo-6-(trifluoromethyl)-1,2-dihydropyridine-3-carboxamide (407) was obtained using the method described for Example 405, except (2S,6S)-6-((4-bromophenoxy)methyl)-2-(methoxymethyl)-2-methyl-1,4-dioxane was replaced with (2R,6R)-6-((4-bromophenoxy)methyl)-2-(methoxymethyl)-2-methyl-1,4-dioxane. LCMS t=0.88 min, m/z=457.4 [M+H]. ¹H NMR (500 MHz, DMSO-d6) δ 13.63 (br. s, 1H), 8.46 (br. s, 1H), 8.27 (s, 1H), 8.17 (br. s, 1H), 7.26 (d, J=8.4 Hz, 2H), 7.03 (d, J=8.4 Hz, 2H), 4.21-4.15 (m, 1H), 4.00-3.91 (m, 2H), 3.88 (dd, J=11.2, 2.8 Hz, 1H), 3.51 (d, J=11.2 Hz, 1H), 3.36 (d, J=11.2 Hz, 2H), 3.31-3.24 (m, 5H), 3.22 (s, 2H).

(2R,6R)-6-((4-bromophenoxy)methyl)-2-(methoxymethyl)-2-methyl-1,4-dioxane was obtained using the method described for the preparation of (2S,6S)-6-((4-bromophenoxy)methyl)-2-(methoxymethyl)-2-methyl-1,4-dioxane, except (S)-2-((4-bromophenoxy)methyl)oxirane was replaced with (R)-2-((4-bromophenoxy)methyl)oxirane.

### Example 408: 5-(4-(((2R,6S)-6-(methoxymethyl)-6-methyl-1,4-dioxan-2-yl)methoxy)phenyl)-2-oxo-6-(trifluoromethyl)-1,2-dihydropyridine-3-carboxamide (408)

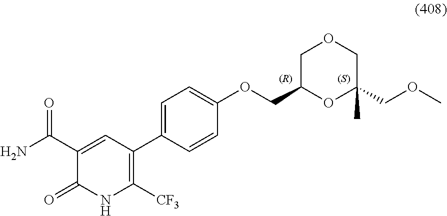

5-(4-(((2R,6S)-6-(methoxymethyl)-6-methyl-1,4-dioxan-2-yl)methoxy)phenyl)-2-oxo-6-(trifluoromethyl)-1,2-dihydropyridine-3-carboxamide (408) was obtained using the method described for Example 405, except (2S,6S)-6-((4-bromophenoxy)methyl)-2-(methoxymethyl)-2-methyl-1,4-dioxane was replaced with (2S,6R)-6-((4-bromophenoxy)methyl)-2-(methoxymethyl)-2-methyl-1,4-dioxane. LCMS t=0.86 min, m/z=457.5 [M+H]. ¹H NMR (500 MHz, DMSO-d₆) δ 13.6 (br. s, 1H), 8.46 (s, 1H), 8.28 (br. s, 1H), 8.18 (s, 1H), 7.30-7.22 (m, 2H), 7.06-7.00 (m, 2H), 4.18 (dtd, J=10.4, 4.9, 3.0 Hz, 1H), 3.99-3.92 (m, 2H), 3.89 (dd, J=11.3, 3.0 Hz, 1H), 3.67 (d, J=11.3 Hz, 1H), 3.64 (d, J=9.5 Hz, 1H), 3.48 (d, J=9.5 Hz, 1H), 3.32-3.26 (m, 1H), 3.15 (d, J=11.6 Hz, 1H), 1.04 (s, 3H).

(2S,6R)-6-((4-bromophenoxy)methyl)-2-(methoxymethyl)-2-methyl-1,4-dioxane was obtained using the method described for the preparation of (2R,6S)-6-((4-bromophenoxy)methyl)-2-(methoxymethyl)-2-methyl-1,4-dioxane, except (S)-2-((4-bromophenoxy)methyl)oxirane was replaced with (R)-2-((4-bromophenoxy)methyl)oxirane

### Example 409: 5-(4-(((2S,6R)-6-(dimethylcarbamoyl)-6-methyl-1,4-dioxan-2-yl)methoxy)phenyl)-2-oxo-6-(trifluoromethyl)-1,2-dihydropyridine-3-carboxamide (409)

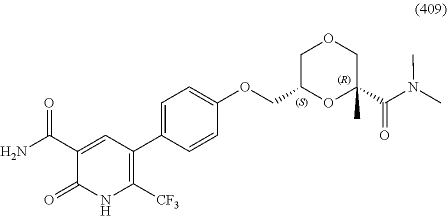

5-(4-(((2S,6R)-6-(dimethylcarbamoyl)-6-methyl-1,4-dioxan-2-yl)methoxy)phenyl)-2-oxo-6-(trifluoromethyl)-1,2-dihydropyridine-3-carboxamide (409) was obtained by RP-HPLC purification of the product obtained using the method described for Example 405, except (2S,6S)-6-((4-bromophenoxy)methyl)-2-(methoxymethyl)-2-methyl-1,4-dioxane was replaced with (6S)-6-((4-bromophenoxy)methyl)-N,N,2-trimethyl-1,4-dioxane-2-carboxamide. ¹H NMR (500 MHz, DMSO-d6) δ 6=9.44 (m, 1H), 8.10 (br s, 1H), 7.75 (br s, 1H), 7.20 (s, 2H), 6.99 (d, J=8.6 Hz, 2H), 4.03 (m, 1H), 4.08 (m, 2H), 3.90 (m, 1H), 3.68 (m, 2H), 3.18 (s, 2H), 2.77 (s, 3H), 1.51 (s, 3H). LCMS t=0.75 min, m/z=484.4 [M+H]. Chiral-HPLC: Rt=1.32 min, ee=100%.

### (6S)-6-((4-bromophenoxy)methyl)-N,N,2-trimethyl-1,4-dioxane-2-carboxamide was Obtained Using the Following Procedure

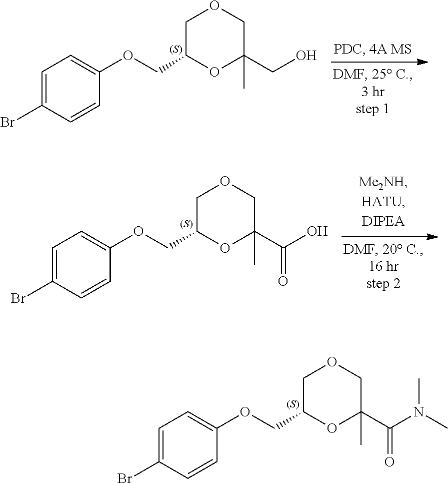

Step 1: To a solution of ((6S)-6-((4-bromophenoxy)methyl)-2-methyl-1,4-dioxan-2-yl)methanol (250 mg, 0.788 mmol) in DMF (4 mL) was added PDC (2.19 g, 5.83 mmol) and MS 4A (750 mg) under N₂ at 25° C. The mixture was stirred at 25° C. for 18 hrs. Water (30 mL) was added and the resulting mixture was filtered. The filtrate was extracted by EA (20 mL×3). The organic layers were combined, filtered, dried over anhydrous Na₂SO₄ and concentrated in vacuo. The residue was purified by reverse phase (FA, ACN: H2O=0.05-0.95) to give (6S)-6-((4-bromophenoxy)methyl)-2-methyl-1,4-dioxane-2-carboxylic acid. LCMS: Rt=0.771 min, [M+Na]*=353.0/355.0. ¹H NMR (400 MHz, CHLOROFORM-d) δ=7.41 (m, 2H), 6.80 (d, J=8.4 Hz, 2H), 4.55 (m, 1H), 4.03 (m, 4H), 3.61 (m, 8H), 1.69 (s, 2H).

Step 2: To a solution of (6S)-6-((4-bromophenoxy)methyl)-2-methyl-1,4-dioxane-2-carboxylic acid (240 mg, 0.725 mmol) in DMF (3 mL) was added HATU (551 mg, 1.45 mmol), DIPEA (281 mg, 2.175 mmol) and dimethylamine (1.1 mL, 2.175 mmol) under N₂ at 20° C. The mixture was stirred at 20° C. for 16 hrs. The mixture was diluted with H₂O (20 mL), extracted with EA (20 mL×3). The organic layers were combined, dried over Na₂SO₄, filtered and concentrated in vacuo. The residue was purified by reverse phase (FA, ACN:H2O=0.05˜0.95) to give I (6S)-6-((4-bromophenoxy)methyl)-N,N,2-trimethyl-1,4-dioxane-2-carboxamide. LCMS: Rt=0.905 min, [M+H]+=358.2. ¹H NMR (400 MHz, CHLOROFORM-d) δ=8.09 (m, 1H), 7.39 (m, 2H), 6.79 (m, 2H), 4.46 (m, 1H), 4.07 (m, 4H), 3.87 (m, 2H), 3.41 (m, 2H), 3.23 (m, 4H), 1.69 (m, 2H), 1.37 (s, 1H).

### Example 410: 5-(4-(((2S,6S)-6-((methoxy-d3)methyl)-6-methyl-1,4-dioxan-2-yl)methoxy)phenyl)-2-oxo-6-(trifluoromethyl)-1,2-dihydropyridine-3-carboxamide (410)

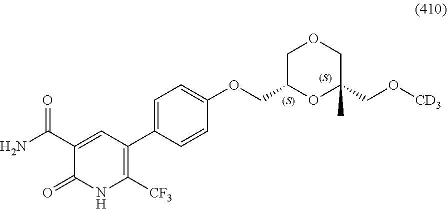

5-(4-(((2S,6S)-6-((methoxy-d3)methyl)-6-methyl-1,4-dioxan-2-yl)methoxy)phenyl)-2-oxo-6-(trifluoromethyl)-1,2-dihydropyridine-3-carboxamide (410) was obtained using the method described for Example 405, except (2S,6S)-6-((4-bromophenoxy)methyl)-2-(methoxymethyl)-2-methyl-1,4-dioxane was replaced with (2S,6S)-6-((4-bromophenoxy)methyl)-2-((methoxy-d3)methyl)-2-methyl-1,4-dioxane. LCMS t=0.75 min, m/z=460.2 [M+H]. ¹H NMR (500 MHz, DMSO-d6) δ 13.64 (br. s, 1H), 8.46 (br. s, 1H), 8.27 (s, 1H), 8.17 (br. s, 1H), 7.26 (d, J=8.4 Hz, 2H), 7.02 (d, J=8.4 Hz, 2H), 4.18 (dtd, J=10.5, 5.0, 3.0 Hz, 1H), 3.99-391 (m, 2H), 3.88 (dd, J=11.2, 3.0 Hz, 1H), 3.51 (d, J=11.2 Hz, 1H), 3.36 (d, J=11.0 Hz, 1H), 3.27 (t, J=11.0 Hz, 1H), 3.22 (s, 2H), 1.29 (s, 3H).

(2S,6S)-6-((4-bromophenoxy)methyl)-2-((methoxy-d3)methyl)-2-methyl-1,4-dioxane was obtained using the method to obtain (2S,6S)-6-((4-bromophenoxy)methyl)-2-(methoxymethyl)-2-methyl-1,4-dioxane except iodomethane was replaced with D3-iodomethane.

### Example 411: 5-(4-(((2S,6S)-6-(cyanomethyl)-6-methyl-1,4-dioxan-2-yl)methoxy)phenyl)-2-oxo-6-(trifluoromethyl)-1,2-dihydropyridine-3-carboxamide (411)

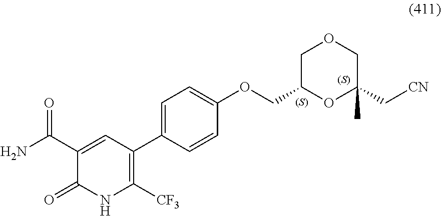

5-(4-(((2S,6S)-6-(cyanomethyl)-6-methyl-1,4-dioxan-2-yl)methoxy)phenyl)-2-oxo-6-(trifluoromethyl)-1,2-dihydropyridine-3-carboxamide (411) was obtained by RP-HPLC purification of the product obtained using the method described for Example 405, except (2S,6S)-6-((4-bromophenoxy)methyl)-2-(methoxymethyl)-2-methyl-1,4-dioxane was replaced with 2-((2S,6S)-6-((4-bromophenoxy)methyl)-2-methyl-1,4-dioxan-2-yl)acetonitrile. ¹H NMR (500 MHz, DMSO-d6) δ=13.63 (br s, 1H), 8.70 (br s, 1H), 8.21 (br s, 1H), 8.04 (br s, 1H), 7.25 (d, J=8.4 Hz, 2H), 7.03 (d, J=8.4 Hz, 2H), 4.22 (m, 1H), 3.99 (m, 3H), 3.60 (m, 1H), 3.31 (m, 2H), 2.83 (m, 2H), 1.40 (s, 3H). LCMS t=0.74 min, m/z=452.3 [M+H]. Chiral-HPLC: Rt=1.58 min, ee=96%.

### 2-((2S,6S)-6-((4-bromophenoxy)methyl)-2-methyl-1,4-dioxan-2-yl)acetonitrile was Obtained Using the Following Procedure

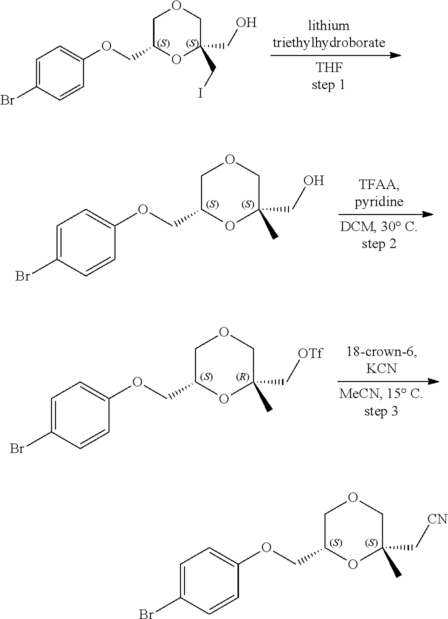

Step 1: To a stirring solution of ((2S,6S)-6-((4-bromophenoxy)methyl)-2-(iodomethyl)-1,4-dioxan-2-ylmethanol (100 mg, 0.226 mmol) in dry THF (1 mL) under N₂ was added lithium triethylhydroborate (0.7 mL, 0.700 mmol) (1M solution in THF) dropwise at rt. The mixture was then heated to 60° C. for 30 min. The mixture was cooled to rt and the reaction quenched with aqueous NaHCO₃. The mixture was acidified with 1M aqueous HCl and extracted with EtOAc. The organic layer was dried over sodium sulfate and concentrated to give ((2S,6S)-6-((4-bromophenoxy)methyl)-2-methyl-1,4-dioxan-2-yl)methanol. LCMS: Rt=0.68 min with weak m/z=317.2 for [M+H]+. Rf=0.45 in 50% EtOAc/heptane

Step 2: To a solution of ((2S,6S)-6-((4-bromophenoxy)methyl)-2-methyl-1,4-dioxan-2-yl)methanol (350 mg, 1.10 mmol) in anhydrous DCM (5 mL) was added successively pyridine (131 mg, 1.66 mmol) and trifluoromethanesulfonic anhydride (623 mg, 2.21 mmol) at 0° C. The reaction mixture was stirred at 30° C. for 20 hrs. The mixture was washed with water (3 mL) and brine (3 mL). The combined aqueous layers were extracted with DCM (3×2 mL). The combined organic layers were dried over anhydrous Na₂SO₄, filtered and concentrated. The residue was purified by silica gel column chromatography (PE/EA=10/1) to give ((2R,6S)-6-((4-bromophenoxy)methyl)-2-methyl-1,4-dioxan-2-yl)methyl trifluoromethanesulfonate. TLC: PE/EA=3/1, Rf=0.7. LCMS: Rt=0.950 min, [M+H]⁺=449.0, 451.0. ¹H NMR (400 MHz, CDCl₃) δ=7.31 (d, J=9.2 Hz, 2H), 6.70 (d, J=8.8 Hz, 2H), 4.30 (m, 1H), 4.21 (m, 2H), 3.93 (m, 2H), 3.76 (m, 1H), 3.54 (m, 1H), 3.46 (m, 1H), 3.32 (m, 1H), 1.38 (s, 3H),

Step 3: To a solution of ((2R,6S)-6-((4-bromophenoxy)methyl)-2-methyl-1,4-dioxan-2-yl)methyl trifluoromethanesulfonate (440 mg, 0.98 mmol) in MeCN (7 mL) was added KCN (128 mg, 1.96 mmol) and 18-crown-6-ether (518 mg, 1.96 mmol) at 15° C. under N₂. The reaction mixture was stirred at 15° C. under N₂ for 17 hrs. The mixture diluted with water (7 mL) and extracted with EA (2×10 mL). The combined organic layers were dried over anhydrous Na₂SO₄, filtered and concentrated to give 2-((2S,6S)-6-((4-bromophenoxy)methyl)-2-methyl-1,4-dioxan-2-yl)acetonitrile. TLC: PE/EA=2/1, Rf=0.5. LCMS: Rt=0.845 min, [M+Na]r=348.0, 350.0. ¹H NMR (400 MHz, CDCl₃) δ=7.39 (d, J=9.2 Hz, 2H), 6.79 (d, J=8.8 Hz, 2H), 4.27 (m, 1H), 4.02 (m, 2H), 3.86 (m, 1H), 3.68 (m, 1H), 3.48 (m, 1H), 3.41 (m, 1H), 2.54 (m, 2H), 1.54 (s, 3H).

### Example 412: 5-(4-(((2S,6R)-6-methyl-6-((methylsulfonyl)methyl)-1,4-dioxan-2-yl)methoxy)phenyl)-2-oxo-6-(trifluoromethyl)-1,2-dihydropyridine-3-carboxamide (412)

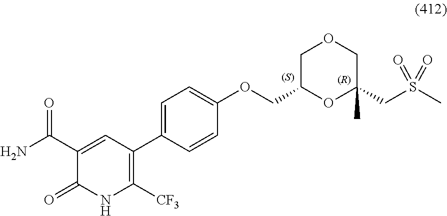

5-(4-(((2S,6R)-6-methyl-6-((methylsulfonyl)methyl)-1,4-dioxan-2-yl)methoxy)phenyl)-2-oxo-6-(trifluoromethyl)-1,2-dihydropyridine-3-carboxamide (412) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-methyl-2-((methylsulfonyl)methyl)-1,4-dioxane. LCMS t=0.69 min, m/z=505.1 [M+H]. ¹H NMR (500 MHz, DMSO-d6) δ 13.65 (br. s, 1H), 8.45 (br. s, 1H), 8.27 (s, 1H), 8.17 (br. s, 1H), 7.27 (d, J=8.3 Hz, 2H), 7.03 (d, J=8.3 Hz, 2H), 4.25-4.18 (m, 1H), 4.00 (d, J=4.7 Hz, 2H), 3.90 (dd, J=11.3, 3.0 Hz, 1H), 3.63 (d, J=11.4 Hz, 1H), 3.54 (d, J=11.4 Hz, 1H), 3.45-3.24 (m, 3H), 3.02 (s, 3H), 1.50 (s, 3H).

### (2R,6S)-6-((4-bromophenoxy)methyl)-2-methyl-2-((methylsulfonyl)methyl)-1,4-dioxane was Obtained Using the Following Procedure

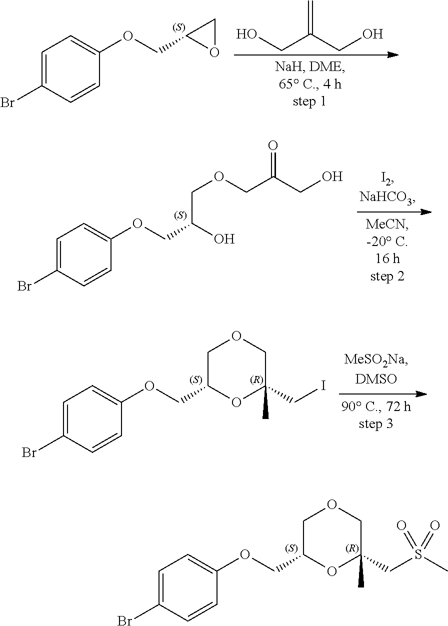

Step 1: To a 500 mL flame-dried 3 necked flask with a stirbar was added 2-methylenepropane-1,3-diol (45.6 ml, 559 mmol) and the vessel was purged with vac/N₂ (×3). DME (186 mL) was added and the reaction cooled to 0° C. Sodium hydride (60% in mineral oil, 4.92 g, 123 mmol) was added in 3 portions over 15 mins and the reaction was stirred at rt for a further 15 mins. A dropping funnel was attached and a solution of (S)-2-((4-bromophenoxy)methyl)oxirane (25.6 g, 112 mmol) in DME (60 mL) was charged to it. The reaction was heated to 60° C. (internal temp) and the solution of epoxide was added dropwise over 1 h. Following completion of addition, the reaction was stirred at 60° C. for a further 3 h. The reaction was quenched with H₂O, extracted with EA (×4), dried over Na₂SO₄, filtered and concentrated in vacuo. Purification by silica gel chromatography (ISCO, 0-100% EA/Heptane) gave (S)-2-((3-(4-bromophenoxy)-2-hydroxypropoxy)methyl)prop-2-en-1-ol. LCMS t=0.69 min, m/z=317.1, 319.1 [M+H].

Step 2: To a 1 L oven-dried 3-necked flask with overhead stirrer was added NaHCO_{3(aq)} (19.7 g, 235 mmol) and iodine (59.5 g, 235 mmol). The reaction was purged with vac/N₂ (×3). Acetonitrile (521 mL) was added and the reaction was stirred at rt for 10 mins before cooling to −20° C. A solution of (S)-2-((3-(4-bromophenoxy)-2-hydroxypropoxy)methyl)prop-2-en-1-ol (24.8 g, 78 mmol) in Et₂O (20 mL) was added and the mixture stirred at −20° C. overnight. The reaction was quenched with Na₂S2O₃ and extracted with EA×4. The combined organic layers were washed with brine, dried over Na₂SO₄, filtered and concentrated in vacuo to give crude product. An initial recrystallization from −3:1 EtOH/H2O gives >95% pure material in a 92:8 dr which was then recrystallised via layer diffusion crystallization between DCM and heptane to give ((2S,6S)-6-((4-bromophenoxy)methyl)-2-(iodomethyl)-1,4-dioxan-2-yl)methanol. LCMS: t=0.89 min, m/z=443.3, 445.3 [M+H].

Step 3: To an 8 ml vial was added ((2S,6S)-6-((4-bromophenoxy)methyl)-2-(iodomethyl)-1,4-dioxan-2-yl)methanol (85 mg, 0.20 mmol) and sodium methanesulfinate (102 mg, 1.0 mmol). The vial was sealed with a septa-top cap and purged with vac/N₂ (×3). DMSO (0.66 mL) was added and the reaction was heated to 90° C. and stirred for 72 h. H₂O and brine were added and the reaction was extracted with EA×4. The combined organic layers were dried over Na₂SO₄, filtered through a plug of silica with EA and concentrated in vacuo. Et₂O was added and crystallisation gave (2R,6S)-6-((4-bromophenoxy)methyl)-2-methyl-2-((methylsulfonyl)methyl)-1,4-dioxane. LCMS t=0.81 min, m/z=379.0, 381.0 [M+H].

### Example 413: 5-(4-(((2S,6R)-6-((cyclopropylsulfonyl)methyl)-6-methyl-1,4-dioxan-2-yl)methoxy)phenyl)-2-oxo-6-(trifluoromethyl)-1,2-dihydropyridine-3-carboxamide (413)

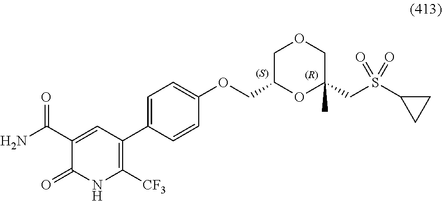

5-(4-(((2S,6R)-6-((cyclopropylsulfonyl)methyl)-6-methyl-1,4-dioxan-2-yl)methoxy)phenyl)-2-oxo-6-(trifluoromethyl)-1,2-dihydropyridine-3-carboxamide (413) was obtained using the method described for Example 412, except (2R,6S)-6-((4-bromophenoxy)methyl)-2-methyl-2-((methylsulfonyl)methyl)-1,4-dioxane was replaced with (2R,6S)-6-((4-bromophenoxy)methyl)-2-((cyclopropylsulfonylmethyl)-2-methyl-1,4-dioxane. LCMS t=0.73 min, m/z=531.2 [M+H]. ¹H NMR (500 MHz, DMSO-d6) δ 13.63 (br. s, 1H), 8.45 (br. s, 1H), 8.27 (s, 1H), 8.16 (br. s, 1H), 7.26 (d, J=8.3 Hz, 2H), 7.01 (d, J=8.3 Hz, 2H), 4.28-4.17 (m, 1H), 4.00 (d, J=4.4 Hz, 2H), 3.94-3.86 (m, 1H), 3.66-3.54 (m, 2H), 3.41-3.28 (m, 1H), 2.81 (tt, J=8.2, 5.0 Hz, 1H), 1.53 (s, 3H), 1.02-0.94 (m, 2H), 0.91-0.81 (m, 2H).

### Example 414: (S)-5-(4-((6,6-bis(methoxymethyl)-1,4-dioxan-2-yl)methoxy)phenyl)-2-oxo-6-(trifluoromethyl)-1,2-dihydropyridine-3-carboxamide (414)

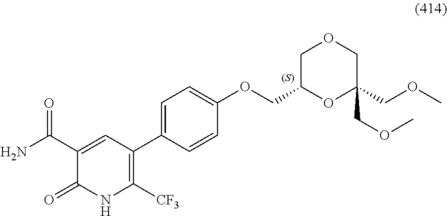

(S)-5-(4-((6,6-bis(methoxymethyl)-1,4-dioxan-2-yl)methoxy)phenyl)-2-oxo-6-(trifluoromethyl)-1,2-dihydropyridine-3-carboxamide (414) was obtained using the method described for Example 403, except (2S,6S)-6-((4-bromophenoxy)methyl)-2-(methoxymethyl)-2-methyl-1,4-dioxane was replaced with (S)-6-((4-bromophenoxy)methyl)-2,2-bis(methoxymethyl)-1,4-dioxane. LCMS t=0.75 min, m/z=487.2 [M+H]. ¹H NMR (500 MHz, DMSO-d6) δ 13.65 (br. s, 1H), 8.59 (br. s, 1H), 8.13 (s, 1H), 7.79 (br. s, 1H), 7.20 (d, J=8.5 Hz, 2H), 7.04-6.86 (m, 2H), 3.91 (d, J=4.6 Hz, 2H), 3.81 (dd, J=11.4, 2.9 Hz, 1H), 3.63 (d, J=10.3 Hz, 1H), 3.58 (d, J=11.4 Hz, 1H), 3.50 (d, J=10.3 Hz, 1H), 3.33 (dd, J=11.6, 9.7 Hz, 2H), 3.28 (d, J=10.3 Hz, 1H), 3.25 (s, 3H), 3.20 (d, J=10.3 Hz, 1H), 3.19 (s, 3H), 1.96 (s, 3H).

### (S)-6-((4-bromophenoxy)methyl)-2,2-bis(methoxymethyl)-1,4-dioxane was Obtained Using the Following Procedure

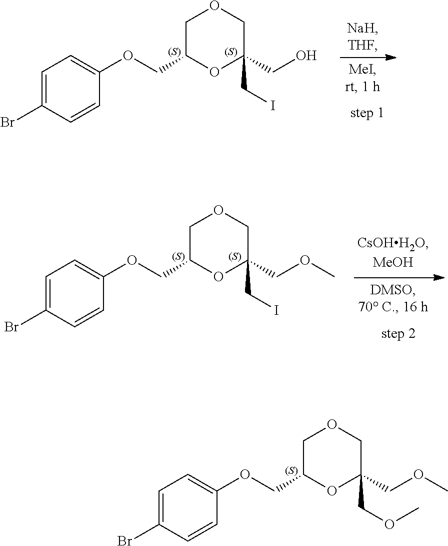

Step 1: To an oven-dried 4 mL vial with a stirbar was added ((2S,6S)-6-((4-bromophenoxy)methyl)-2-(iodomethyl)-1,4-dioxan-2-yl)methanol (100 mg, 0.23 mmol) and sodium hydride (60% in mineral oil, 11.7 mg, 0.29 mmol). The reaction vial was sealed with a septa-top cap and purged with vac/N₂ (×3). THF (1.1 mL) was added and the reaction mixture was stirred at rt for 10 mins. Iodomethane (71 μL, 1.13 mmol) was then added and the reaction mixture stirred at rt for 1 h. The reaction was quenched with methanol, filtered through a plug of SiO₂ with Et₂O and concentrated in vacuo to give (2S,6S)-6-((4-bromophenoxy)methyl)-2-(iodomethyl)-2-(methoxymethyl)-1,4-dioxane which was used without further purification. LCMS t=1.06 min, m/z=457.1, 459.1 [M+H].

Step 2: To an oven-dried 4 mL vial with a stirbar was added (2S,6S)-6-((4-bromophenoxy)methyl)-2-(iodomethyl)-2-(methoxymethyl)-1,4-dioxane (103 mg, 0.23 mmol) and cesium hydroxide monohydrate (150 mg, 0.90 mmol). The vial was sealed with a septa-top cap and purged with vac/N₂ (×3). DMSO (1.1 mL) was added, followed by methanol (73 μL, 1.80 mmol) and the reaction mixture heated to 70° C. and stirred overnight. The reaction mixture was cooled to rt, quenched with NH₄Cl(aq) and extracted into Et₂O (×4). The combined organic layers were dried over Na₂SO₄, filtered and concentrated in vacuo. The residue was purified by silica gel chromatography (ISCO, 0-70% EA/heptane) to give (S)-6-((4-bromophenoxy)methyl)-2,2-bis(methoxymethyl)-1,4-dioxane. LCMS t=0.90 min, m/z=361.1, 363.1 [M+H].

### Example 415: (S)-5-(4-((2,5,8-trioxaspiro[3.5]nonan-6-yl)methoxy)phenyl)-2-oxo-6-(trifluoromethyl)-1,2-dihydropyridine-3-carboxamide (415)

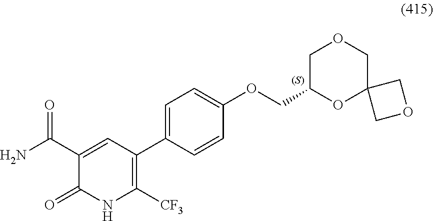

(S)-5-(4-((2,5,8-trioxaspiro[3.5]nonan-6-yl)methoxy)phenyl)-2-oxo-6-(trifluoromethyl)-1,2-dihydropyridine-3-carboxamide (415) was obtained using the method described for Example 405, except (2S,6S)-6-((4-bromophenoxy)methyl)-2-(methoxymethyl)-2-methyl-1,4-dioxane was replaced with (S)-6-((4-bromophenoxy)methyl)-2,5,8-trioxaspiro[3.5]nonane. LCMS t=0.65 min, m/z=441.2 [M+H]. ¹H NMR (500 MHz, DMSO-d₆) δ 13.62 (br. s, 1H), 8.44 (br. s, 1H), 8.26 (s, 1H), 8.15 (br. s, 1H), 7.26 (d, J=8.5 Hz, 2H), 7.03 (d, J=8.5 Hz, 2H), 6.50 (br. s, 1H), 4.58 (dd, J=6.7, 1.7 Hz, 1H), 4.53 (d, J=6.7 Hz, 1H), 4.32 (d, J=7.2 Hz, 1H), 4.30 (d, J=7.2 Hz, 1H), 4.08 (d, J=11.7 Hz, 1H), 4.06-3.99 (m, 2H), 3.96-3.88 (m, 1H), 3.81 (dd, J=11.7, 2.7 Hz, 1H), 3.49-3.38 (m, 2H).

### (S)-6-((4-bromophenoxy)methyl)-2,5,8-trioxaspiro[3.5]nonane was Obtained Using the Following Procedure

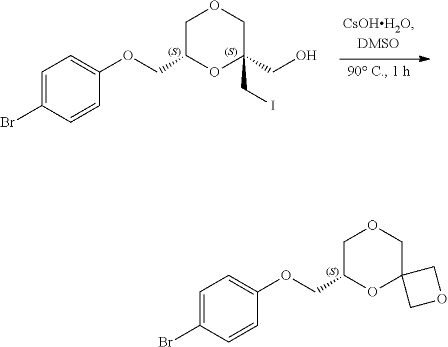

To a 4 mL vial with a stirbar was added ((2S,6S)-6-((4-bromophenoxy)methyl)-2-(iodomethyl)-1,4-dioxan-2-yl)methanol (100 mg, 0.23 mmol) and cesium hydroxide monohydrate (49 mg, 0.29 mmol). The vial was sealed with a septa-top cap and purged with vac/N₂ (×3). DMSO (2.2 mL) was added and the reaction mixture stirred at 90° C. for 1 h. The reaction was quenched with 1M HCl, extracted with Et₂O (×3), dried over Na₂SO₄, filtered through a plug of SiO₂ and concentrated in vacuo. Purification of the residue by reverse phase HPLC, followed by lyophilisation, gave (S)-6-((4-bromophenoxy)methyl)-2,5,8-trioxaspiro[3.5]nonane. LCMS t=0.80 min, m/z=315.1, 317.1 [M+H].

### Example 416: (S)-5-(4-((1,4,8,11-tetraoxaspiro[5.6]dodecan-2-yl)methoxy)phenyl)-2-oxo-6-(trifluoromethyl)-1,2-dihydropyridine-3-carboxamide (416)

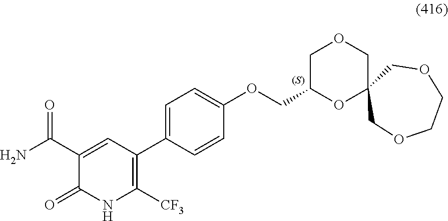

(S)-5-(4-((1,4,8,11-tetraoxaspiro[5.6]dodecan-2-yl)methoxy)phenyl)-2-oxo-6-(trifluoromethyl)-1,2-dihydropyridine-3-carboxamide (416) was obtained using the method described for Example 405, except (2S,6S)-6-((4-bromophenoxy)methyl)-2-(methoxymethyl)-2-methyl-1,4-dioxane was replaced with (S)-2-((4-bromophenoxy)methyl)-1,4,8,11-tetraoxaspiro[5.6]dodecane. LCMS t=0.70 min, m/z=485.2 [M+H].

### (S)-2-((4-bromophenoxy)methyl)-1,4,8,11-tetraoxaspiro[5.6]dodecane was Obtained Using the Following Procedure

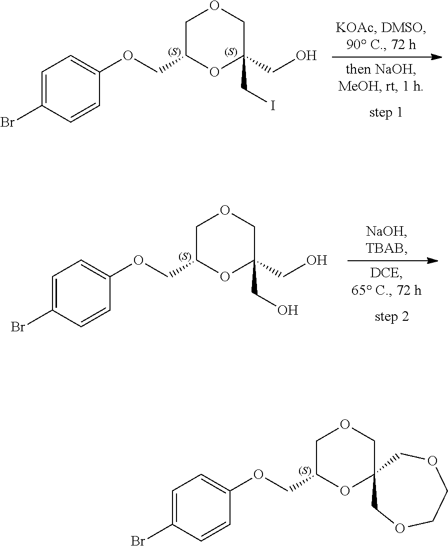

Step 1: To an oven-dried 4 mL vial with a stirbar was added ((2S,6S)-6-((4-bromophenoxy)methyl)-2-(iodomethyl)-1,4-dioxan-2-yl)methanol (0.14 g, 0.32 mmol) and potassium acetate (0.155 g, 1.58 mmol). The vial was sealed with a septa-top cap and purged with vac/N₂ (×3). DMSO (1.6 mL) was added and the reaction mixture heated to 90° C. and stirred for 72 h. The reaction was quenched with H₂O and brine, extracted into EA (×4), dried over Na₂SO₄, filtered through a plug of silica with EA and concentrated in vacuo. The residue was dissolved in MeOH (1.5 mL) and sodium hydroxide (36 mg, 0.90 mmol) was added. The reaction mixture then stirred at rt for 1 h. The reaction mixture was concentrated and the residue was redissolved in 1 M HCl and extracted into EA. The combined organic layers were dried over Na₂SO₄, filtered through a plug of silica and concentrated to give (S)-(6-((4-bromophenoxy)methyl)-1,4-dioxane-2,2-diyl)dimethanol which was used without further purification. LCMS t=0.65 min, m/z=333.1, 335.1 [M+H].

Step 2: To a 10 mL MW vial with a stirbar was added (S)-(6-((4-bromophenoxy)methyl)-1,4-dioxane-2,2-diyl)dimethanol (100 mg, 0.300 mmol), sodium hydroxide (720 mg, 18.0 mmol) and TBAB (19.4 mg, 0.06 mmol). The vial was sealed and purged with vac/N₂×3. DCE (1.5 mL) and an equal amount of water was added and the reaction heated to 65° C. and stirred vigorously overnight. An additional amount of DCE (1.5 mL) was added and the reaction was stirred at 65° C. for a further 48 h. The reaction was then diluted with water and extracted in Et₂O (×4). The combined organic layers were dried over Na₂SO₄, filtered and concentrated in vacuo. Purification of the residue by reverse phase HPLC gave (S)-2-((4-bromophenoxy)methyl)-1,4,8,11-tetraoxaspiro[5.6]dodecane. LCMS t=0.85 min, m/z=359.1, 361.1 [M+H].

### Example 417: 5-(4-(((2S,6S)-6-(fluoromethyl)-6-(methoxymethyl)-1,4-dioxan-2-yl)methoxy)phenyl)-2-oxo-6-(trifluoromethyl)-1,2-dihydropyridine-3-carboxamide (417)

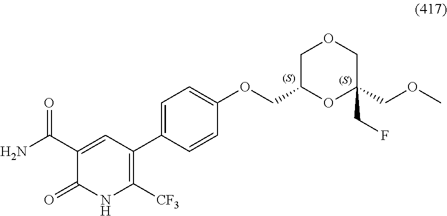

5-(4-(((2S,6S)-6-(fluoromethyl)-6-(methoxymethyl)-1,4-dioxan-2-yl)methoxy)phenyl)-2-oxo-6-(trifluoromethyl)-1,2-dihydropyridine-3-carboxamide (417) was obtained using the method described for Example 405, except (2S,6S)-6-((4-bromophenoxy)methyl)-2-(methoxymethyl)-2-methyl-1,4-dioxane was replaced with (2S,6S)-6-((4-bromophenoxy)methyl)-2-(fluoromethyl)-2-(methoxymethyl)-1,4-dioxane. LCMS t=0.76 min, m/z=475.2 [M+H]. ¹H NMR (500 MHz, DMSO-d₆) δ 13.65 (br. s, 1H), 8.46 (br. s, 1H), 8.27 (s, 1H), 8.17 (br. s, 1H), 7.26 (d, J=8.3 Hz, 2H), 7.03 (d, J=8.3 Hz, 2H), 4.83 (dd, J=48.2, 9.9 Hz, 1H), 4.60 (dd, J=46.0, 10.0 Hz, 1H), 4.31-4.22 (m, 1H), 4.04-3.95 (m, 2H), 3.92 (dd, J=11.4, 3.0 Hz, 1H), 3.70 (d, J=12.1 Hz, 1H), 3.43 (dd, J=12.1, 6.4 Hz, 1H), 3.37 (dd, J=12.5, 10.3 Hz, 2H), 3.25 (dd, J=9.9, 1.9 Hz, 1H).

### (2S,6S)-6-((4-bromophenoxy)methyl)-2-(fluoromethyl)-2-(methoxymethyl)-1,4-dioxane was Obtained Using the Following Procedure

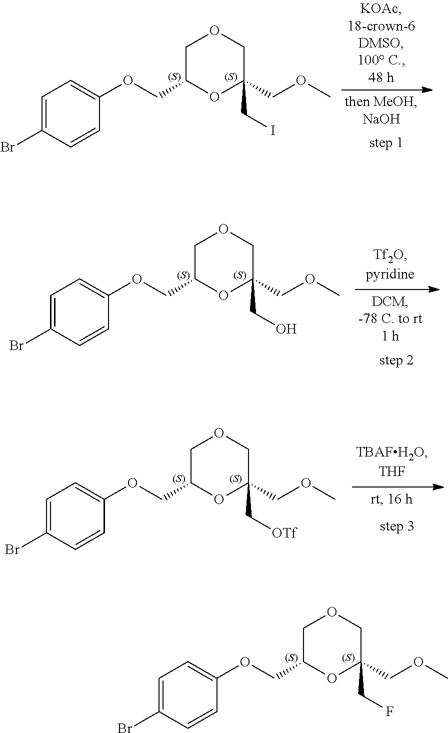

Step 1: To an oven-dried 4 mL vial with a stirbar was added (2S,6S)-6-((4-bromophenoxy)methyl)-2-(iodomethyl)-2-(methoxymethyl)-1,4-dioxane (see Example 412) (560 mg, 1.22 mmol), 18-crown-6 (97 mg, 0.37 mmol) and potassium acetate (601 mg, 6.1 mmol). The vial was sealed with a septa-top cap and purged with vac/N₂ (×3). DMSO (8.2 mL) was added and the reaction mixture was heated to 100° C. and stirred for 48 h. The reaction was diluted with EA (250 mL), and washed sequentially with H₂O, 1:1 H₂O/Brine and then brine. The organic layer was dried over Na₂SO₄, filtered through a plug of SiO₂ with EA, concentrated in vacuo. The residue was dissolved in MeOH (6.1 mL) and sodium hydroxide (294 mg, 7.35 mmol) was added. The vial was sealed and stirred at rt for 30 mins. The reaction was quenched with AcOH, diluted with EA and filtered through a plug of SiO₂. The concentrated eluent was then purified by silica gel chromatography (ISCO, 0-100% EA/Heptane) to give ((2S,6S)-6-((4-bromophenoxy)methyl)-2-(methoxymethyl)-1,4-dioxan-2-yl)methanol. LCMS t=0.75, m/z=347.1, 349.1 [M+H].

Step 2: To an oven-dried 8 mL vial with a stirbar was added ((2S,6S)-6-((4-bromophenoxy)methyl)-2-(methoxymethyl)-1,4-dioxan-2-yl)methanol (130 mg, 0.37 mmol). The vial was sealed with a septa-top cap and purged with vac/N₂ (×3). DCM (3.7 mL) and pyridine (61 μL, 0.75 mmol) were added and the reaction was cooled to −78° C. Tf₂O (89 μl, 0.52 mmol) was added dropwise and the reaction mixture was then warmed to rt and stirred for 1 h. NaHCO_{3(aq)} was added to quench the reaction, and the mixture was then extracted into Et₂O (×3). The combined organic layers were dried over Na₂SO₄, filtered and concentrated in vacuo to give ((2S,6S)-6-((4-bromophenoxy)methyl)-2-(methoxymethyl)-1,4-dioxan-2-yl)methyl trifluoromethanesulfonatel which was used without further purification. LCMS t=1.07 min, m/z=479.1, 481.0 [M+H].

Step 3: To an oven-dried 4 mL vial with a stirbar was added ((2S,6S)-6-((4-bromophenoxy)methyl)-2-(methoxymethyl)-1,4-dioxan-2-yl)methyl trifluoromethanesulfonate (177 mg, 0.37 mmol) and the vial was sealed with a septa-top cap and purged with vac/N₂ (×3). TBAF·3H₂O (1M in THF, 740 μl, 0.74 mmol) was added and the reaction mixture stirred at rt overnight. The reaction mixture was diluted with Et₂O, SiO₂ was added and the mixture stirred vigorously for 5 mins before filtering through a plug of SiO₂ with Et₂O. The filtrate was concentrated in vacuo to give (2S,6S)-6-((4-bromophenoxy)methyl)-2-(fluoromethyl)-2-(methoxymethyl)-1,4-dioxane which was used without further purification. LCSM t=0.90 min, m/z=349.1, 351.1 [M+H].

### Example 418: 5-(4-(((2S,6S)-6-(chloromethyl)-6-(methoxymethyl)-1,4-dioxan-2-yl)methoxy)phenyl)-2-oxo-6-(trifluoromethyl)-1,2-dihydropyridine-3-carboxamide (418)

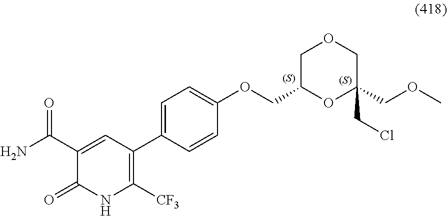

5-(4-(((2S,6S)-6-(chloromethyl)-6-(methoxymethyl)-1,4-dioxan-2-yl)methoxy)phenyl)-2-oxo-6-(trifluoromethyl)-1,2-dihydropyridine-3-carboxamide (418) was obtained using the method described for Example 417, except (2S,6S)-6-((4-bromophenoxy)methyl)-2-(fluoromethyl)-2-(methoxymethyl)-1,4-dioxane was replaced with (2S,6S)-6-((4-bromophenoxy)methyl)-2-(chloromethyl)-2-(methoxymethyl)-1,4-dioxane. LCMS t=0.82 min, m/z=491.2 [M+H]. ¹H NMR (500 MHz, DMSO-d₆) δ 13.61 (br. s, 1H), 8.46 (br. s, 1H), 8.27 (s, 1H), 8.17 (br. s, 1H), 7.26 (d, J=8.4 Hz, 2H), 7.03 (d, J=8.4 Hz, 2H), 4.22-4.15 (m, 1H), 4.15 (d, J=12.0 Hz, 1H), 4.04-3.96 (m, 2H), 3.92 (dd, J=11.4, 2.9 Hz, 1H), 3.84 (d, J=11.9 Hz, 1H), 3.72 (d, J=12.0 Hz, 1H), 3.49 (d, J=12.0 Hz, 1H), 3.41-3.36 (m, 1H), 3.25 (d, J=9.8 Hz, 1H).

(2S,6S)-6-((4-bromophenoxy)methyl)-2-(chloromethyl)-2-(methoxymethyl)-1,4-dioxane was obtained using the procedure used in the synthesis of (2S,6S)-6-((4-bromophenoxy)methyl)-2-(fluoromethyl)-2-(methoxymethyl)-1,4-dioxane, except tetrabutylammonium chloride (TBAF) was replaced with tetrabutylammonium chloride (TBAC).

### Example 419: 5-(4-(((2S,6S)-6-(fluoromethyl)-6-methyl-1,4-dioxan-2-yl)methoxy)phenyl)-2-oxo-6-(trifluoromethyl)-1,2-dihydropyridine-3-carboxamide (419)

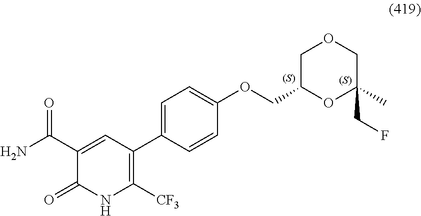

5-(4-(((2S,6S)-6-(fluoromethyl)-6-methyl-1,4-dioxan-2-yl)methoxy)phenyl)-2-oxo-6-(trifluoromethyl)-1,2-dihydropyridine-3-carboxamide (419) was obtained using the method described for Example 405, except (2S,6S)-6-((4-bromophenoxy)methyl)-2-(methoxymethyl)-2-methyl-1,4-dioxane was replaced with (2S,6S)-6-((4-bromophenoxy)methyl)-2-(fluoromethyl)-2-methyl-1,4-dioxane. LCMS t=0.76 min, m/z=445.3 [M+H]. ¹H NMR (400 MHz, DMSO-d6) δ=13.64 (br s, 1H), 8.51 (br s, 1H), 8.26 (s, 1H), 8.16 (br s, 1H), 7.27 (d, J=8.8 Hz, 2H), 7.03 (d, J=8.8 Hz, 2H), 4.77 (m, 2H), 4.52 (m, 1H), 3.98 (m, 2H), 3.95 (m, 1H), 3.72 (m 1H), 3.36 (m, 2H), 3.33 (m, 1H), 1.08 (s, 3H).

### Example 420: 5-(4-(((2S,6R)-6-(fluoromethyl)-6-methyl-1,4-dioxan-2-yl)methoxy)phenyl)-2-oxo-6-(trifluoromethyl)-1,2-dihydropyridine-3-carboxamide (420)

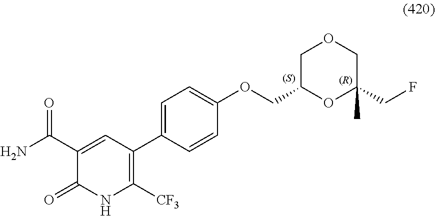

5-(4-(((2S,6R)-6-(fluoromethyl)-6-methyl-1,4-dioxan-2-yl)methoxy)phenyl)-2-oxo-6-(trifluoromethyl)-1,2-dihydropyridine-3-carboxamide (420) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-(fluoromethyl)-2-methyl-1,4-dioxane. LCMS t=0.77 min, m/z=445.0 [M+H]. ¹H NMR (400 MHz, DMSO-d6) δ=13.65 (br s, 1H), 8.49 (br s, 1H), 8.26 (s, 1H), 8.17 (br s, 1H), 7.27 (d, J=8.4 Hz, 2H), 7.04 (d, J=8.8 Hz, 2H), 4.33 (s, 1H), 4.21 (s, 1H), 3.98 (m, 2H), 3.96 (m, 1H), 3.58 (d, J=11.2 Hz, 1H), 3.43 (d, J=11.2 Hz, 1H), 3.33 (m, 2H), 1.29 (d, J=2.0 Hz, 3H).

### Example 421: 5-(4-(((2S,6R)-6-((3-fluoroazetidin-1-yl)methyl)-6-methyl-1,4-dioxan-2-ylmethoxy)phenyl)-2-oxo-6-(trifluoromethyl)-1,2-dihydropyridine-3-carboxamide (421)

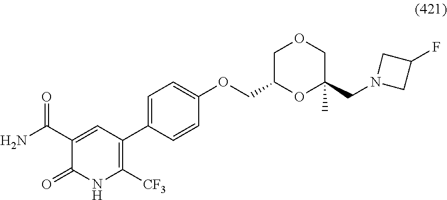 [SMILES: C[C@@]1(CN2CC(F)C2)COC[C@@H](COc2ccc(-c3cc(C(N)=O)c(=O)[nH]c3C(F)(F)F)cc2)O1]

5-(4-(((2S,6R)-6-((3-fluoroazetidin-1-yl)methyl)-6-methyl-1,4-dioxan-2-ylmethoxy)phenyl)-2-oxo-6-(trifluoromethyl)-1,2-dihydropyridine-3-carboxamide (421) was obtained using the method described for Example 405, except (2S,6S)-6-((4-bromophenoxy)methyl)-2-(methoxymethyl)-2-methyl-1,4-dioxane was replaced with 1-(((2R,6S)-6-((4-bromophenoxy)methyl)-2-methyl-1,4-dioxan-2-yl)methyl)-3-fluoroazetidine. ¹H NMR: (400 MHz DMSO-d6), δ=8.65 (s, 1H), 8.23 (s, 1H), 8.08 (s, 1H), 7.25 (d, J=8.8 Hz, 2H), 7.00 (d, J=8.4 Hz, 2H), 5.28-5.02 (m, 1H), 4.26-4.16 (m, 1H), 3.97-3.82 (m, 3H), 3.79-3.57 (m, 5H), 3.33-3.20 (m, 5H), 3.17-3.08 (m, 2H), 2.95-2.73 (m, 3H), 1.01 (s, 3H). LCMS: Rt=0.58 min, m/z=500.1 (M+H). Chiral HPLC: Rt=2.31 min, ee value=100%.

### 1-(((2R,6S)-6-((4-bromophenoxy)methyl)-2-methyl-1,4-dioxan-2-yl)methyl)-3-fluoroazetidine was Obtained Using the Following Procedure

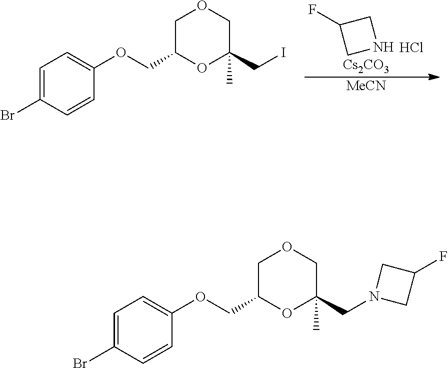

To a solution of (2S,6S)-6-((4-bromophenoxy)methyl)-2-(Iodomethyl)-2-methyl-1,4-dioxane (2.0 g, 4.68 mmol) in MeCN (20 mL) were added 3-fluoroazetidine hydrochloride (1.04 g, 9.37 mmol) and Cs₂CO₃ (4.58 g, 14.05 mmol) at 25° C. and the mixture was stirred at 120° C. for 6 days. The reaction mixture was poured into water (40 mL) and the resulting mixture extracted with EA (20 mL×3). The combined organic layers were dried over anhydrous Na₂SO₄, filtered and concentrated. The crude product was purified by silica gel column chromatography (PE/EA=50/1 to 5/1) to give 1-(((2R,6S)-6-((4-bromophenoxy)methyl)-2-methyl-1,4-dioxan-2-yl)methyl)-3-fluoroazetidine. ¹H NMR (400 MHz, CDCl₃) δ=7.41-7.34 (m, 2H), 6.81-6.72 (m, 2H), 5.18 (m, 1H), 5.04 (m, 1H), 4.29-4.19 (m, 1H), 3.99-3.90 (m, 2H), 3.85-3.65 (m, 4H), 3.37-3.18 (m, 4H), 2.95-2.78 (m, 2H), 1.09 (s, 3H).

### Example 422: 5-(4-(((2S,6R)-6-((3,3-difluoroazetidin-1-yl)methyl)-6-methyl-1,4-dioxan-2-yl)methoxy)phenyl)-2-oxo-6-(trifluoromethyl)-1,2-dihydropyridine-3-carboxamide (422)

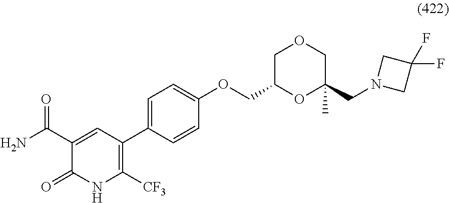 [SMILES: C[C@@]1(CN2CC(F)(F)C2)COC[C@@H](COc2ccc(-c3cc(C(N)=O)c(=O)[nH]c3C(F)(F)F)cc2)O1]

5-(4-(((2S,6R)-6-((3,3-difluoroazetidin-1-yl)methyl)-6-methyl-1,4-dioxan-2-yl)methoxy)phenyl)-2-oxo-6-(trifluoromethyl)-1,2-dihydropyridine-3-carboxamide (422) was obtained using the method described for Example 405, except (2S,6S)-6-((4-bromophenoxy)methyl)-2-(methoxymethyl)-2-methyl-1,4-dioxane was replaced with 1-(((2R,6S)-6-((4-bromophenoxy)methyl)-2-methyl-1,4-dioxan-2-yl)methyl)-3,3-difluoroazetidine. ¹H NMR: (400 MHz, DMSO-d6), δ=8.72 (br s, 1H), 8.20 (s, 1H), 8.01 (br s, 1H), 7.25 (d, J=8.8 Hz, 2H), 7.00 (d, J=8.8 Hz, 2H), 4.24 (m, 1H), 3.94 (m, 2H), 3.86 (m, 1H), 3.68-3.62 (m, 5H), 3.27-3.25 (m, 3H), 3.16 (m, 1H), 3.16 (m, 1H), 2.81 (m, 1H), 1.03 (s, 3H). LCMS: Rt=0.77 min, m/z=518.4 (M+H). Chiral HPLC: Rt=1.67 min, ee value=100%.

1-(((2R,6S)-6-((4-bromophenoxy)methyl)-2-methyl-1,4-dioxan-2-yl)methyl)-3,3-difluoroazetidine was obtained using the method in the synthesis of 1-(((2R,6S)-6-((4-bromophenoxy)methyl)-2-methyl-1,4-dioxan-2-yl)methyl)-3-fluoroazetidine, except 3-fluoroazetidine hydrochloride was replaced with 2,2-difluoroazetidine hydrochloride.

### Example 423: 5-(4-(((2S,6S)-6-((dimethylamino)methyl)-6-methyl-1,4-dioxan-2-yl)methoxy)phenyl)-2-oxo-6-(trifluoromethyl)-1,2-dihydropyridine-3-carboxamide (423)

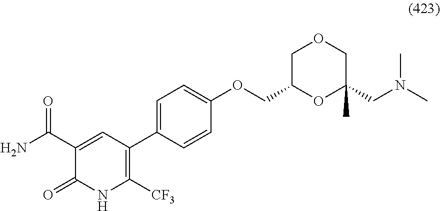 [SMILES: CN(C)C[C@@]1(C)COC[C@@H](COc2ccc(-c3cc(C(N)=O)c(=O)[nH]c3C(F)(F)F)cc2)O1]

5-(4-(((2S,6S)-6-((dimethylamino)methyl)-6-methyl-1,4-dioxan-2-yl)methoxy)phenyl)-2-oxo-6-(trifluoromethyl)-1,2-dihydropyridine-3-carboxamide (423) was obtained using the method described for Example 405, except (2S,6S)-6-((4-bromophenoxy)methyl)-2-(methoxymethyl)-2-methyl-1,4-dioxane was replaced with 1-((2S,6S)-6-((4-bromophenoxy)methyl)-2-methyl-1,4-dioxan-2-yl)-N,N-dimethylmethanamine. ¹H NMR (400 MHz, DMSO-d6) δ=9.29 (br s, 1H), 8.10 (s, 1H), 7.76 (br s, 1H), 7.20 (d, J=8.6 Hz, 2H), 6.98 (d, J=8.6 Hz, 2H), 4.14 (m, 1H), 3.95 (m, 2H), 3.89 (m, 2H), 3.46 (m, 2H), 3.31 (m, 1H), 2.57 (s, 1H), 2.44 (s, 6H), 1.34 (s, 3H). LCMS: Rt=0.78 min, m/z=470.4 (M+H). Chiral HPLC: Rt=3.303 min, de value=95%.

### 1-((2S,6S)-6-((4-bromophenoxy)methyl)-2-methyl-1,4-dioxan-2-yl)-N,N-dimethylmethanamine was Obtained Using the Following Procedure

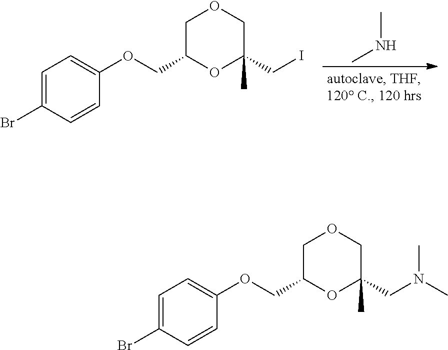

A solution of (2R,6S)-6-((4-bromophenoxy)methyl)-2-(iodomethyl)-2-methyl-1,4-dioxane (2.0 g, 4.68 mmol) and dimethylamine in THF (2M, 14.5 mL) was heated in an 250-mL autoclave at 120° C. for 5 days. The reaction mixture was concentrated in vacuum and the residue purified by silica gel column chromatography (PE-PE/EA=1/1) to give 1-((2S,6S)-6-((4-bromophenoxy)methyl)-2-methyl-1,4-dioxan-2-yl)-N,N-dimethylmethanamine. LCMS: Rt=0.61 min, m/z=344.0, 348.0 (M+H).

### Example 424: 5-(4-(((2S,6R)-6-((dimethylamino)methyl)-6-methyl-1,4-dioxan-2-yl)methoxy)phenyl)-2-oxo-6-(trifluoromethyl)-1,2-dihydropyridine-3-carboxamide (424)

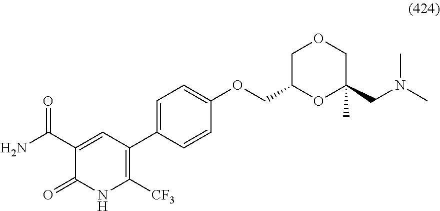 [SMILES: CN(C)C[C@]1(C)COC[C@@H](COc2ccc(-c3cc(C(N)=O)c(=O)[nH]c3C(F)(F)F)cc2)O1]

5-(4-(((2S,6R)-6-((dimethylamino)methyl)-6-methyl-1,4-dioxan-2-yl)methoxy)phenyl)-2-oxo-6-(trifluoromethyl)-1,2-dihydropyridine-3-carboxamide (424) was obtained using the method described for Example 405, except (2S,6S)-6-((4-bromophenoxy)methyl)-2-(methoxymethyl)-2-methyl-1,4-dioxane was replaced with 1-((2R,6S)-6-((4-bromophenoxy)methyl)-2-methyl-1,4-dioxan-2-yl)-N,N-dimethylmethanamine. ¹H NMR (400 MHz, CDCl3) δ=9.48 (brs, 1H), 8.06 (s, 1H), 7.65 (brs, 1H), 7.20 (d, J=8.0 Hz, 2H), 6.96 (d, J=8.0 Hz, 2H), 4.35 (m, 1H), 3.97 (m, 3H), 3.66 (m, 1H), 3.29 (m, 3H), 2.83 (m, 1H), 2.55 (s, 6H), 1.16 (s, 3H). LCMS: Rt=0.61 min, m/z=470.1 (M+H). Chiral HPLC: Rt=3.178 min, de value=>99%.

### Example 425: 5-(4-(((2S,6R)-6-((difluoromethoxy)methyl)-6-methyl-1,4-dioxan-2-yl)methoxy)phenyl)-2-oxo-6-(trifluoromethyl)-1,2-dihydropyridine-3-carboxamide (425)

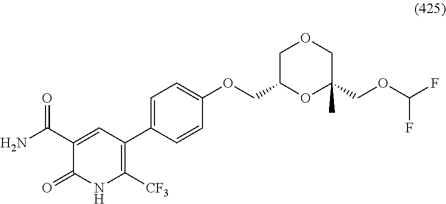 [SMILES: C[C@]1(COC(F)F)COC[C@@H](COc2ccc(-c3cc(C(N)=O)c(=O)[nH]c3C(F)(F)F)cc2)O1]

5-(4-(((2S,6R)-6-((difluoromethoxy)methyl)-6-methyl-1,4-dioxan-2-yl)methoxy)phenyl)-2-oxo-6-(trifluoromethyl)-1,2-dihydropyridine-3-carboxamide (425) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-((difluoromethoxy)methyl)-2-methyl-1,4-dioxane. ¹H NMR: (400 MHz, DMSO-d6), δ=13.6 (br s, 1H), 8.53 (br s, 1H), 8.25 (s, 1H), 5 8.15 (br s, 1H), 7.26 (d, J=8.6 Hz, 2H), 7.03 (d, J=8.8 Hz, 2H), 6.68 (m, 1H), 4.19 (m, 1H), 3.99 (m, 2H), 3.91 (m, 1H), 3.75 (m, 2H), 3.56 (m, 1H), 3.40 (m, 1H), 3.29 (m, 1H), 1.31 (s, 3H). LCMS: Rt=0.89 min, m/z=493.0 (M+H). Chiral HPLC: Rt=2.03 min, de value=99.14%.

### (2R,6S)-6-((4-bromophenoxy)methyl)-2-((difluoromethoxy)methyl)-2-methyl-1,4-dioxane was Obtained Using the Following Procedure

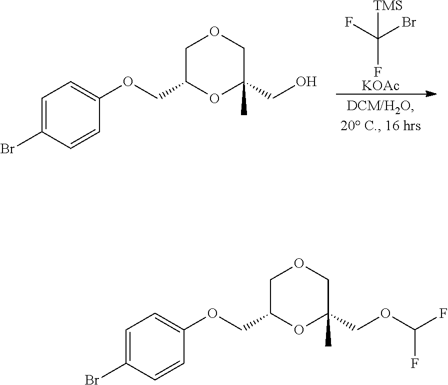 [SMILES: C[C@]1(CO)COC[C@@H](COc2ccc(Br)cc2)O1 ; C[Si](C)(C)C(F)(F)Br ; C[C@]1(COC(F)F)COC[C@@H](COc2ccc(Br)cc2)O1 ; "KOAc"]

To a solution of ((2S,6S)-6-((4-bromophenoxy)methyl)-2-methyl-1,4-dioxan-2-yl)methanol (800 mg, 2.52 mmol) and KOAc (990 mg, 10.09 mmol) in 1:1 DCM/H₂O (7 mL) was added (bromodifluoromethyl)trimethylsilane (1025 mg, 5.04 mmol) at 0° C. The reaction mixture was warmed to 20° C. and stirred at 20° C. for 16 hrs. The mixture was diluted with water (10 mL) and extracted with DCM (15 mL×2). The combined organic layers were washed with brine, dried over anhydrous Na₂SO₄, filtered and concentrated. The residue was purified by column chromatography (PE/EA=100/1-20/1) to give (2R,6S)-6-((4-bromophenoxy)methyl)-2-((difluoromethoxy)methyl)-2-methyl-1,4-dioxane. ¹H NMR (400 MHz, CDCl₃) δ=7.38 (d, J=9.2 Hz, 2H), 6.78 (d, J=8.8 Hz, 2H), 6.44 (m, 1H), 4.26 (m, 1H), 3.99 (m, 2H), 3.84 (m, 1H), 3.75 (m, 2H), 3.62 (m, 1H), 3.50 (m, 1H), 3.39 (m, 1H), 1.42 (s, 3H).

### Example 426: 5-(4-(((2S,6S)-6-(2-methoxyethyl)-6-methyl-1,4-dioxan-2-yl)methoxy)phenyl)-2-oxo-6-(trifluoromethyl)-1,2-dihydropyridine-3-carboxamide (426)

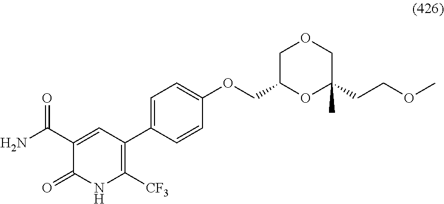 [SMILES: COCC[C@@]1(C)COC[C@@H](COc2ccc(-c3cc(C(N)=O)c(=O)[nH]c3C(F)(F)F)cc2)O1]

5-(4-(((2S,6S)-6-(2-methoxyethyl)-6-methyl-1,4-dioxan-2-yl)methoxy)phenyl)-2-oxo-6-(trifluoromethyl)-1,2-dihydropyridine-3-carboxamide (426) was obtained using the method described for Example 405, except (2S,6S)-6-((4-bromophenoxy)methyl)-2-(methoxymethyl)-2-methyl-1,4-dioxane was replaced with (2S,6S)-6-((4-bromophenoxy)methyl)-2-(2-methoxyethyl)-2-methyl-1,4-dioxane. ¹H NMR (500 MHz, DMSO-d₆) δ 13.64 (s, 1H), 8.47 (s, 1H), 8.29 (s, 1H), 8.19 (s, 1H), 7.28 (d, J=8.5 Hz, 2H), 7.05 (d, J=8.6 Hz, 2H), 4.17 (ddd, J=10.5, 7.9, 4.6 Hz, 1H), 3.97 (qd, J=10.4, 4.9 Hz, 2H), 3.89 (dd, J=11.2, 3.0 Hz, 1H), 3.51 (d, J=11.3 Hz, 1H), 3.44 (td, J=6.8, 2.9 Hz, 2H), 3.27 (dt, J=11.1, 5.3 Hz, 2H), 3.22 (s, 3H), 1.72-1.57 (m, 2H), 1.31 (s, 3H). LCMS Rt=3.30 min, m/z=471.2 (M+H).

### (2S,6S)-6-((4-bromophenoxy)methyl)-2-(2-methoxyethyl)-2-methyl-1,4-dioxane was Obtained Using the Following Procedure

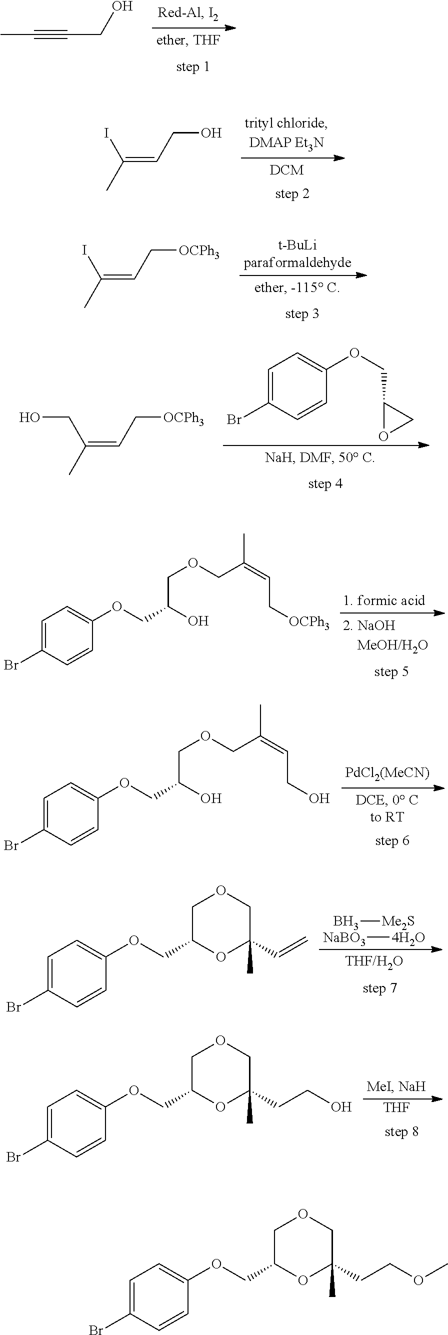

Step 1: An oven-dried 1000 mL flask was cooled under N₂ and then charged with dry ether (120 mL) and 2-butyne-1-ol (9.25 mL, 124 mmol). The solution was cooled to 0° C., and Red-Al (53 mL, 157 mmol) (60 wt % solution in toluene) was added dropwise over 20 min. The reaction mixture was warmed to RT and stirred for 15 hours. The reaction mixture was then cooled to 0° C. and a THF solution (120 mL) of iodine (40.8 g, 161 mmol) was added. The reaction mixture was warmed to RT, stirred for 30 min and the reaction quenched with 10% aqueous sodium sulphite. 1M Rochelle's salt solution (150 mL) was added and the suspension vigorously stirred for 30 min. The crude mixture was extracted with EtOAc twice, dried over sodium sulfate, and concentrated. The crude was then purified via silica gel chromatography (ISCO, 0 to 30% ethyl acetate/heptane) to give (Z)-3-iodobut-2-en-1-ol (17.52 g, 88 mmol, 72% yield). ¹H NMR (500 MHz, CDCl₃) δ 5.80 (t, J=6.2 Hz, 1H), 4.19 (t, J=6.1 Hz, 2H), 2.56 (s, 3H), 1.59 (t, J=6.0 Hz, 1H).

Step 2: To a 500 mL flask was dissolved (Z)-3-iodobut-2-en-1-ol (17.5 g, 88 mmol) and trityl chloride (30.5 g, 106 mmol) in dry DCM (150 mL). The mixture was cooled to 0° C., then triethylamine (18.5 mL, 133 mmol) was added, followed by DMAP (1.080 g, 8.84 mmol). The reaction was warmed to RT and stirred for 16 hours. The reaction was filtered, washed with 1M HCl and sat. NaHCO₃, dried over sodium sulfate, and concentrated. The crude was then purified via silica gel chromatography twice (ISCO, 0 to 10% ethyl acetate/heptane) to give (Z)-(((3-iodobut-2-en-1-yl)oxy)methanetriyl)tribenzene. LCMS Rt=1.20 min, m/z=441.1 (M+H).

Step 3: To an oven-dried 500 mL flask cooled under N₂ was added (Z)-(((3-iodobut-2-en-1-yl)oxy)methanetriyl)tribenzene (22.4 g, 50.8 mmol) in dry Et₂O (160 mL). After cooling to around −115° C. with a liquid N₂/Et₂O bath, tert-butyllithium (66 mL, 112 mmol) was then added dropwise over 30 minutes. The reaction was kept at −115° C. for 1 hour, then solid paraformaldehyde (4.58 g, 152 mmol) was added, and the reaction slowly warmed to RT over 1 hour, and then stirred at RT for 2 hours. The reaction was quenched with sat. NH₄Cl, extracted with EtOAc, dried over sodium sulfate, and concentrated. The crude was then purified via silica gel chromatography (ISCO, 0 to 30% ethyl acetate/heptane) to give (Z)-2-methyl-4-(trityloxy)but-2-en-1-ol. LCMS Rt=0.98 min, m/z=367.3 (M+Na).

Step 4: To a 500 mL flask was added (S)-2-((4-bromophenoxy)methyl)oxirane (3.6 g, 15.7 mmol) and (Z)-2-methyl-4-(trityloxy)but-2-en-1-ol (10.8 g, 31.4 mmol) in dry DMF (32 mL). After purging with N₂ for a few minutes, the mixture was cooled to 0° C., and sodium hydride (0.794 g, 31.4 mmol) was added in one portion. The flask was then capped with a rubber septum, stirred at RT for 15 min and then heated to 50° C. for 1 hour. The reaction was diluted with 1M HCl (35 mL) and water (120 mL), extracted with EtOAc, washed with brine, dried over sodium sulfate, and concentrated to give crude (S,Z)-1-(4-bromophenoxy)-3-((2-methyl-4-(trityloxy)but-2-en-1-yl)oxy)propan-2-ol which was used without further purification. LCMS Rt=1.17 min, m/z=595.2 (M+Na).

Step 5: Crude (S,Z)-1-(4-bromophenoxy)-3-((2-methyl-4-(trityloxy)but-2-en-1-yl)oxy)propan-2-ol (9.01 g, 15.7 mmol) was added to a 250 mL flask followed by formic acid (20 mL, 530 mmol) and the mixture was stirred at RT for 30 min. The crude was filtered, concentrated down, and azeotroped several times from DCM. This gave a mixture of around 1:1 (S,Z)-4-(3-(4-bromophenoxy)-2-hydroxypropoxy)-3-methylbut-2-en-1-ol and its formate ester. The crude mixture was dissolved in MeOH (30 mL) and water (15 mL), then powdered NaOH (3.14 g, 79 mmol) was added and the reaction stirred at RT for 30 min. The reaction was filtered, washed with MeOH, and concentrated down. The residue was redissolved in DCM, and acidified to pH 4 with 1M HCl. The aqueous was extracted with DCM, dried over sodium sulfate, and concentrated. The crude was then purified via silica gel chromatography (ISCO, 0 to 100% ethyl acetate/heptane) to give (S,Z)-4-(3-(4-bromophenoxy)-2-hydroxypropoxy)-3-methylbut-2-en-1-ol. LCMS Rt=0.67 min, m/z=353.3, 355.3 (M+Na).

Step 6: To a 250 mL flask was added a solution of (S,Z)-4-(3-(4-bromophenoxy)-2-hydroxypropoxy)-3-methylbut-2-en-1-ol (3.35 g, 10.1 mmol) in dry 1,2-dichloroethane (50 mL). The mixture was cooled to 0° C., then PdCl₂(MeCN) (0.263 g, 1.01 mmol) was added, and the reaction stirred at 0° C. for 1 hour. The reaction was then warmed to RT and stirred for 30 min. The reaction was concentrated down, then purified via silica gel chromatography (ISCO, 0 to 20% ethyl acetate/heptane) to give (2S,6S)-6-((4-bromophenoxy)methyl)-2-methyl-2-vinyl-1,4-dioxane. ¹H NMR (500 MHz, CDCl₃) δ 7.39 (d, J=8.7 Hz, 2H), 6.81 (d, J=8.7 Hz, 2H), 5.83 (dd, J=17.5, 11.0 Hz, 1H), 5.36-5.28 (m, 1H), 5.22-5.15 (m, 1H), 4.31 (ddt, J=10.4, 6.5, 3.4 Hz, 1H), 4.06-3.97 (m, 2H), 3.87 (dd, J=9.8, 6.3 Hz, 1H), 3.56 (d, J=11.3 Hz, 1H), 3.42-3.33 (m, 2H), 1.50 (s, 3H).

Step 7: To a 100 mL flask was added (2S,6S)-6-((4-bromophenoxy)methyl)-2-methyl-2-vinyl-1,4-dioxane (600 mg, 1.92 mmol) and dry THF (4 mL). The flask was purged with N₂, then borane-methyl sulfide complex (6.5 mL, 6.50 mmol, 1M solution in 2-methylTHF) was added dropwise, and the mixture then stirred at RT for 2 hours. Water (5 mL) was carefully added dropwise, then sodium perborate tetrahydrate (3 g, 19.5 mmol) was added, and the reaction continued at RT for 2 hours. The reaction was filtered then concentrated down. The crude was purified via silica gel chromatography (ISCO, 0 to 60% ethyl acetate/heptane) to give 2-((2S,6S)-6-((4-bromophenoxy)methyl)-2-methyl-1,4-dioxan-2-yl)ethan-1-ol. LCMS Rt=0.72 min, m/z=331.3, 333.3 (M+H).

Step 8: To a microwave vial was 2-((2S,6S)-6-((4-bromophenoxy)methyl)-2-methyl-1,4-dioxan-2-yl)ethan-1-ol (37 mg, 0.111 mmol) and dry THF (1 mL). Then sodium hydride (8 mg, 0.332 mmol) was added. The mixture was stirred at RT for 5 min, following which iodomethane (0.035 mL, 0.553 mmol) was added, and the reaction was continued at RT for 68 hours. The reaction was then diluted with 1M HCl, extracted with EtOAc, dried over sodium sulfate, and concentrated to give (2S,6S)-6-((4-bromophenoxy)methyl)-2-(2-methoxyethyl)-2-methyl-1,4-dioxane which was use without further purification. LCMS Rt=0.87 min, m/z=345.3, 347.3 (M+H).

### Example 427: 5-(4-(((2S,6R)-6-methyl-6-((oxetan-3-yloxy)methyl)-1,4-dioxan-2-yl)methoxy)phenyl)-2-oxo-6-(trifluoromethyl)-1,2-dihydropyridine-3-carboxamide (427)

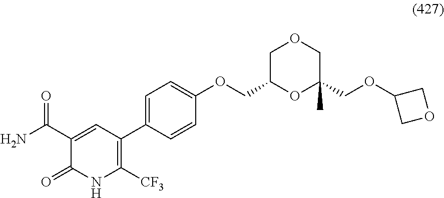 [SMILES: C[C@]1(COC2COC2)COC[C@@H](COc2ccc(-c3cc(C(N)=O)c(=O)[nH]c3C(F)(F)F)cc2)O1]

5-(4-(((2S,6R)-6-methyl-6-((oxetan-3-yloxy)methyl)-1,4-dioxan-2-yl)methoxy)phenyl)-2-oxo-6-(trifluoromethyl)-1,2-dihydropyridine-3-carboxamide (427) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-methyl-2-((oxetan-3-yloxy)methyl)-1,4-dioxane. ¹H NMR (500 MHz, DMSO-d₆) δ 13.64 (s, 1H), 8.70 (s, 1H), 8.23 (s, 1H), 8.05 (s, 1H), 7.26 (d, J=8.7 Hz, 2H), 7.03 (d, J=8.7 Hz, 2H), 4.64 (td, J=6.5, 2.2 Hz, 2H), 4.58-4.52 (m, 1H), 4.44 (ddd, J=15.3, 6.8, 5.1 Hz, 2H), 4.19 (dtd, J=10.5, 4.9, 2.9 Hz, 1H), 4.01-3.93 (m, 2H), 3.90 (dd, J=11.3, 2.9 Hz, 1H), 3.54 (d, J=11.3 Hz, 1H), 3.40 (d, J=11.3 Hz, 1H), 3.32-3.30 (m, 1H), 3.28 (dd, J=9.7, 5.2 Hz, 2H), 1.31 (s, 3H). LCMS Rt=2.98 min, m/z=499.1 (M+H).

### (2R,6S)-6-((4-bromophenoxy)methyl)-2-methyl-2-((oxetan-3-yloxy)methyl)-1,4-dioxane was Obtained Using the Following Procedure

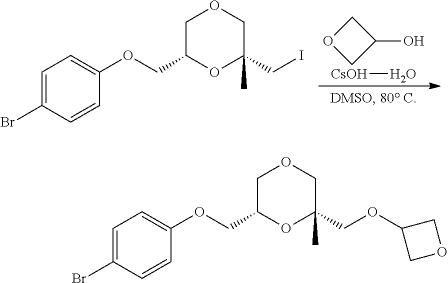

To a microwave vial was added (2R,6S)-6-((4-bromophenoxy)methyl)-2-(iodomethyl)-2-methyl-1,4-dioxane (250 mg, 0.585 mmol) in dry DMSO (3 mL), followed by cesium hydroxide monohydrate (300 mg, 1.80 mmol) and oxetan-3-ol (0.2 mL, 3.15 mmol. The reaction mixture was then heated to 80° C. for 2 hours. The reaction was diluted with water (15 mL), extracted with ether and washed with brine, dried over sodium sulfate, and concentrated. The crude product was then purified via silica gel chromatography (ISCO, 0 to 50% ethyl acetate/heptane) to give (2R,6S)-6-((4-bromophenoxy)methyl)-2-methyl-2-((oxetan-3-yloxy)methyl)-1,4-dioxane. LCMS Rt=0.78 min, m/z=373.2, 375.2 (M+H).

### Example 428: 5-(4-(((2S,6S)-6-(ethoxymethyl)-6-methyl-1,4-dioxan-2-yl)methoxy)phenyl)-2-oxo-6-(trifluoromethyl)-1,2-dihydropyridine-3-carboxamide (428)

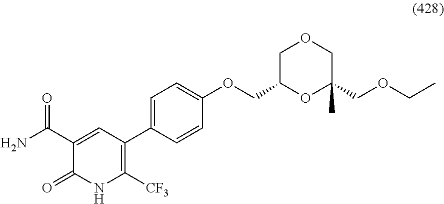 [SMILES: CCOC[C@@]1(C)COC[C@@H](COc2ccc(-c3cc(C(N)=O)c(=O)[nH]c3C(F)(F)F)cc2)O1]

5-(4-(((2S,6S)-6-(ethoxymethyl)-6-methyl-1,4-dioxan-2-yl)methoxy)phenyl)-2-oxo-6-(trifluoromethyl)-1,2-dihydropyridine-3-carboxamide (428) was obtained using the method described for Example 405, except (2S,6S)-6-((4-bromophenoxy)methyl)-2-(methoxymethyl)-2-methyl-1,4-dioxane was replaced with (2S,6S)-6-((4-bromophenoxy)methyl)-2-(ethoxymethyl)-2-methyl-1,4-dioxane. ¹H NMR (500 MHz, DMSO-d₆) δ 13.64 (s, 1H), 8.47 (s, 1H), 8.29 (s, 1H), 8.19 (s, 1H), 7.28 (d, J=8.7 Hz, 2H), 7.04 (d, J=8.7 Hz, 2H), 4.23-4.16 (m, 1H), 4.01-3.93 (m, 2H), 3.90 (dd, J=11.3, 2.9 Hz, 1H), 3.54 (d, J=11.2 Hz, 1H), 3.51-3.41 (m, 2H), 3.37 (d, J=11.9 Hz, 1H), 3.31 (d, J=11.1 Hz, 1H), 3.27 (s, 2H), 1.31 (s, 3H), 1.12 (t, J=7.0 Hz, 3H). LCMS Rt=3.49 min, m/z=471.2 (M+H).

(2S,6S)-6-((4-bromophenoxy)methyl)-2-(ethoxymethyl)-2-methyl-1,4-dioxane was obtained using the method for the synthesis of (2R,6S)-6-((4-bromophenoxy)methyl)-2-methyl-2-((oxetan-3-yloxy)methyl)-1,4-dioxane, except oxetan-3-ol was replaced with ethanol.

### Example 429: 5-(4-(((2S,6R)-6-methyl-6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide (429)

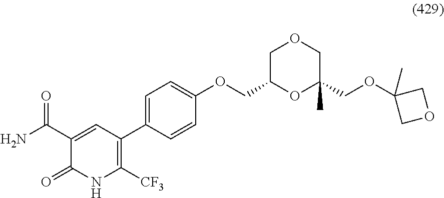 [SMILES: CC1(OC[C@@]2(C)COC[C@@H](COc3ccc(-c4cc(C(N)=O)c(=O)[nH]c4C(F)(F)F)cc3)O2)COC1]

5-(4-(((2S,6R)-6-methyl-6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide (429) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-methyl-2-(((3-methyl)oxetan-3-yl)oxy)methyl)-1,4-dioxane. ¹H NMR (500 MHz, DMSO-d₆) δ 13.65 (s, 1H), 8.47 (s, 1H), 8.29 (s, 1H), 8.19 (s, 1H), 7.28 (d, J=8.4 Hz, 2H), 7.05 (d, J=8.6 Hz, 2H), 4.51 (d, J=6.5 Hz, 2H), 4.26 (d, J=6.4 Hz, 2H), 4.22 (dd, J=10.1, 4.3 Hz, 1H), 4.01-3.94 (m, 2H), 3.91 (dd, J=11.3, 2.9 Hz, 1H), 3.58 (d, J=11.2 Hz, 1H), 3.39 (d, J=11.3 Hz, 1H), 3.30 (d, J=11.1 Hz, 1H), 3.24 (s, 2H), 1.45 (s, 3H), 1.35 (s, 3H). LCMS Rt=3.21 min, m/z=535.3 (M+H).

(2R,6S)-6-((4-bromophenoxy)methyl)-2-methyl-2-(((3-methyl)oxetan-3-yl)oxy)methyl)-1,4-dioxane was obtained using the method for the synthesis of (2R,6S)-6-((4-bromophenoxy)methyl)-2-methyl-2-((oxetan-3-yloxy)methyl)-1,4-dioxane, except oxetan-3-ol was replaced with 3-methyl)oxetan-3-ol.

### Example 430: 5-(4-(((2S,6S)-6-((2-methoxyethoxy)methyl)-6-methyl-1,4-dioxan-2-yl)methoxy)phenyl)-2-oxo-6-(trifluoromethyl)-1,2-dihydropyridine-3-carboxamide (430)

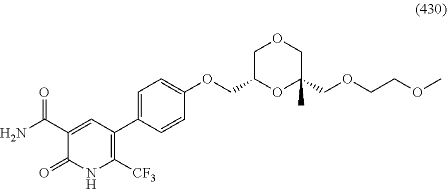 [SMILES: COCCOC[C@@]1(C)COC[C@@H](COc2ccc(-c3cc(C(N)=O)c(=O)[nH]c3C(F)(F)F)cc2)O1]

5-(4-(((2S,6S)-6-((2-methoxyethoxy)methyl)-6-methyl-1,4-dioxan-2-yl)methoxy)phenyl)-2-oxo-6-(trifluoromethyl)-1,2-dihydropyridine-3-carboxamide (430) was obtained using the method described for Example 405, except (2S,6S)-6-((4-bromophenoxy)methyl)-2-(methoxymethyl)-2-methyl-1,4-dioxane was replaced with (2S,6S)-6-((4-bromophenoxy)methyl)-2-((2-methoxyethoxy)methyl)-2-methyl-1,4-dioxane. ¹H NMR (500 MHz, DMSO-d₆) δ 13.66 (s, 1H), 8.47 (s, 1H), 8.29 (s, 1H), 8.19 (s, 1H), 7.28 (d, J=8.5 Hz, 2H), 7.04 (d, J=8.7 Hz, 2H), 4.19 (ddd, J=10.3, 5.3, 3.1 Hz, 1H), 3.97 (qd, J=10.4, 4.9 Hz, 2H), 3.90 (dd, J=11.3, 3.0 Hz, 1H), 3.61-3.51 (m, 3H), 3.45 (t, J=4.7 Hz, 2H), 3.38 (d, J=11.3 Hz, 1H), 3.32 (s, 2H), 3.30 (s, 1H), 3.26 (s, 3H), 1.31 (s, 3H). LCMS Rt=3.11 min, m/z=510.3 (M+H).

(2S,6S)-6-((4-bromophenoxy)methyl)-2-((2-methoxyethoxy)methyl)-2-methyl-1,4-dioxane was obtained using the method for the synthesis of (2R,6S)-6-((4-bromophenoxy)methyl)-2-methyl-2-((oxetan-3-yloxy)methyl)-1,4-dioxane, except oxetan-3-ol was replaced with 2-methoxyethanol.

### Example 431: 5-(4-(((2S,6R)-6-((2,2-difluoroethoxy)methyl)-6-methyl-1,4-dioxan-2-yl)methoxy)phenyl)-2-oxo-6-(trifluoromethyl)-1,2-dihydropyridine-3-carboxamide (431)

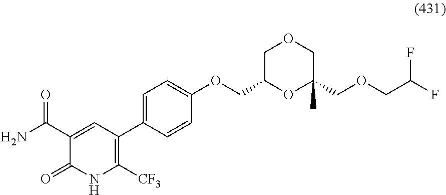 [SMILES: C[C@]1(COCC(F)F)COC[C@@H](COc2ccc(-c3cc(C(N)=O)c(=O)[nH]c3C(F)(F)F)cc2)O1]

5-(4-(((2S,6R)-6-((2,2-difluoroethoxy)methyl)-6-methyl-1,4-dioxan-2-yl)methoxy)phenyl)-2-oxo-6-(trifluoromethyl)-1,2-dihydropyridine-3-carboxamide (431) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-((2,2-difluoroethoxy)methyl)-2-methyl-1,4-dioxane. ¹H NMR (500 MHz, DMSO-d₆) δ 13.64 (s, 1H), 8.51 (s, 1H), 8.28 (s, 1H), 8.17 (s, 1H), 7.28 (d, J=8.5 Hz, 2H), 7.04 (d, J=8.6 Hz, 2H), 6.17 (tt, J=55.0, 3.8 Hz, 1H), 4.20 (dq, J=10.4, 4.4 Hz, 1H), 3.99 (h, J=5.4 Hz, 2H), 3.90 (dd, J=11.3, 2.9 Hz, 1H), 3.85-3.67 (m, 2H), 3.53 (d, J=11.2 Hz, 1H), 3.48 (d, J=10.6 Hz, 1H), 3.44 (d, J=10.5 Hz, 1H), 3.41 (d, J=11.3 Hz, 1H), 3.30 (d, J=11.1 Hz, 1H), 1.31 (s, 3H). LCMS Rt=4.22 min, m/z=507.3 (M+H).

(2R,6S)-6-((4-bromophenoxy)methyl)-2-((2,2-difluoroethoxy)methyl)-2-methyl-1,4-dioxane was obtained using the method for the synthesis of (2R,6S)-6-((4-bromophenoxy)methyl)-2-methyl-2-((oxetan-3-yloxy)methyl)-1,4-dioxane, except oxetan-3-ol was replaced with 2,2-difluoroethanol.

### Example 432: 5-(4-(((2S,6R)-6-methyl-6-((2,2,2-trifluoroethoxy)methyl)-1,4-dioxan-2-yl)methoxy)phenyl)-2-oxo-6-(trifluoromethyl)-1,2-dihydropyridine-3-carboxamide (432)

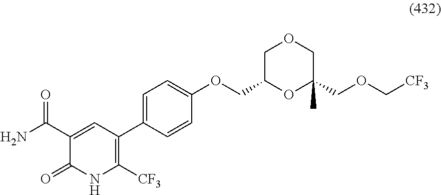 [SMILES: C[C@]1(COCC(F)(F)F)COC[C@@H](COc2ccc(-c3cc(C(N)=O)c(=O)[nH]c3C(F)(F)F)cc2)O1]

5-(4-(((2S,6R)-6-methyl-6-((2,2,2-trifluoroethoxy)methyl)-1,4-dioxan-2-yl)methoxy)phenyl)-2-oxo-6-(trifluoromethyl)-1,2-dihydropyridine-3-carboxamide (432) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-methyl-2-((2,2,2-trifluoroethoxy)methyl)-1,4-dioxane. ¹H NMR (500 MHz, DMSO-d₆) δ 13.65 (s, 1H), 8.47 (s, 1H), 8.29 (s, 1H), 8.19 (s, 1H), 7.28 (d, J=8.3 Hz, 2H), 7.04 (d, J=8.4 Hz, 2H), 4.25-4.06 (m, 3H), 4.04-3.95 (m, 2H), 3.90 (dd, J=11.3, 3.0 Hz, 1H), 3.57-3.49 (m, 3H), 3.42 (d, J=11.3 Hz, 1H), 3.32 (t, J=11.1 Hz, 1H), 1.31 (s, 3H). LCMS Rt=3.89 min, m/z=525.3 (M+H).

(2R,6S)-6-((4-bromophenoxy)methyl)-2-methyl-2-((2,2,2-trifluoroethoxy)methyl)-1,4-dioxane was obtained using the method for the synthesis of (2R,6S)-6-((4-bromophenoxy)methyl)-2-methyl-2-((oxetan-3-yloxy)methyl)-1,4-dioxane, except oxetan-3-ol was replaced with trifluoroethanol.

### Example 433: 5-(4-(((2S,6S)-6-(hydroxymethyl)-6-methyl-1,4-dioxan-2-yl)methoxy)phenyl)-2-oxo-6-(trifluoromethyl)-1,2-dihydropyridine-3-carboxamide (433)

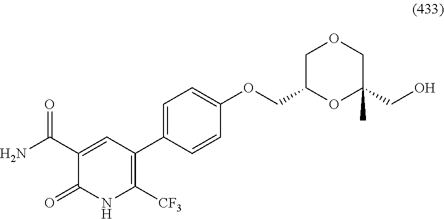 [SMILES: C[C@]1(CO)COC[C@@H](COc2ccc(-c3cc(C(N)=O)c(=O)[nH]c3C(F)(F)F)cc2)O1]

5-(4-(((2S,6S)-6-(hydroxymethyl)-6-methyl-1,4-dioxan-2-yl)methoxy)phenyl)-2-oxo-6-(trifluoromethyl)-1,2-dihydropyridine-3-carboxamide (433) was obtained using the method described for Example 405, except (2S,6S)-6-((4-bromophenoxy)methyl)-2-(methoxymethyl)-2-methyl-1,4-dioxane was replaced with ((2S,6S)-6-((4-bromophenoxy)methyl)-2-methyl-1,4-dioxan-2-ylmethanol. ¹H NMR (500 MHz, DMSO-d6) δ 13.64 (s, 1H), 8.47 (s, 1H), 8.29 (s, 1H), 8.19 (s, 1H), 7.28 (d, J=8.5 Hz, 2H), 7.04 (d, J=8.6 Hz, 2H), 4.19 (ddd, J=10.5, 8.0, 4.5 Hz, 1H), 3.96 (qd, J=10.3, 5.0 Hz, 2H), 3.89 (dd, J=11.2, 3.0 Hz, 1H), 3.54 (d, J=11.3 Hz, 1H), 3.35 (d, J=11.4 Hz, 1H), 3.28 (q, J=11.0 Hz, 3H), 1.28 (s, 3H). LCMS Rt=2.37 min, m/z=443.3 (M+H).

### Example 434: 5-(4-(((2S,6S)-6-(2,2-difluoroethyl)-6-methyl-1,4-dioxan-2-yl)methoxy)phenyl)-2-oxo-6-(trifluoromethyl)-1,2-dihydropyridine-3-carboxamide (434)

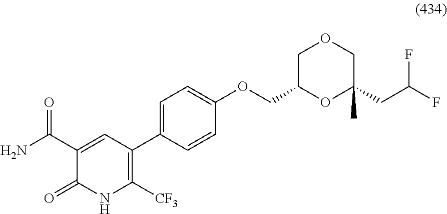 [SMILES: C[C@]1(CC(F)F)COC[C@@H](COc2ccc(-c3cc(C(N)=O)c(=O)[nH]c3C(F)(F)F)cc2)O1]

5-(4-(((2S,6S)-6-(2,2-difluoroethyl)-6-methyl-1,4-dioxan-2-yl)methoxy)phenyl)-2-oxo-6-(trifluoromethyl)-1,2-dihydropyridine-3-carboxamide (434) was obtained using the method described for Example 405, except (2S,6S)-6-((4-bromophenoxy)methyl)-2-(methoxymethyl)-2-methyl-1,4-dioxane was replaced with (2S,6S)-6-((4-bromophenoxy)methyl)-2-(2,2-difluoroethyl)-2-methyl-1,4-dioxane. ¹H NMR (500 MHz, DMSO-d₆) δ 13.62 (s, 1H), 8.48 (s, 1H), 8.28 (s, 1H), 8.18 (s, 1H), 7.28 (d, J=8.6 Hz, 2H), 7.05 (d, J=8.6 Hz, 2H), 6.19 (tt, J=56.2, 4.6 Hz, 1H), 4.21 (ddd, J=10.5, 7.9, 4.6 Hz, 1H), 4.05-3.96 (m, 2H), 3.91 (dd, J=11.3, 3.0 Hz, 1H), 3.54 (d, J=11.3 Hz, 1H), 3.33-3.29 (m, 2H), 2.02 (tt, J=18.4, 4.1 Hz, 2H), 1.38 (s, 3H). LCMS Rt=3.68 min, m/z=477.0 (M+H).

### (2S,6S)-6-((4-bromophenoxy)methyl)-2-(2,2-difluoroethyl)-2-methyl-1,4-dioxane was Obtained Using the Following Procedure

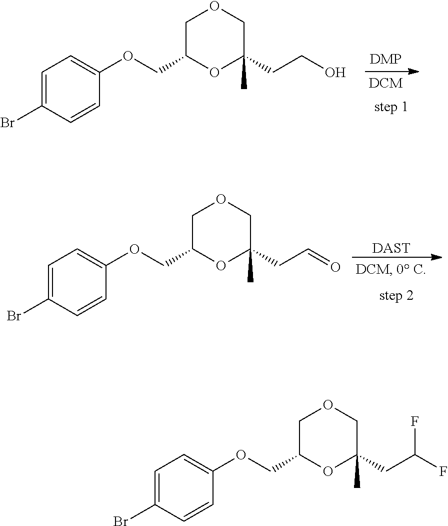

Step 1: To a 100 mL flask was added 2-((2S,6S)-6-((4-bromophenoxy)methyl)-2-methyl-1,4-dioxan-2-yl)ethan-1-ol (336 mg, 1.01 mmol) and dry DCM (5 mL), followed by Dess-Martin periodinane (516 mg, 1.22 mmol) The mixture was stirred at RT for 2 hours. The reaction was quenched with a 1:1 mixture of 1M sodium thiosulfate and sat. NaHCO₃ and then stirred at RT for 20 min. The crude was extracted with DCM, dried over sodium sulfate, and concentrated. The residue was purified via silica gel chromatography (ISCO, 0 to 50% ethyl acetate/heptane) to give 2-((2S,6S)-6-((4-bromophenoxy)methyl)-2-methyl-1,4-dioxan-2-ylacetaldehyde. LCMS Rt=0.78 min, m/z=329.3, 331.3 (M+H).

Step 2: To a stirring solution of 2-((2S,6S)-6-((4-bromophenoxy)methyl)-2-methyl-1,4-dioxan-2-yl)acetaldehyde (135 mg, 0.410 mmol) in dry DCM (3 mL) cooled to 0° C. was added DAST (0.17 mL, 1.29 mmol) dropwise. The reaction was continued at 0° C. for 1 hour. The reaction was quenched with sat. NaHCO₃, extracted with DCM, dried over sodium sulfate, and concentrated to give crude (2S,6S)-6-((4-bromophenoxy)methyl)-2-(2,2-difluoroethyl)-2-methyl-1,4-dioxane (135 mg) that was used without further purification. ¹H NMR (500 MHz, CDCl₃) δ 7.40 (d, J=9.0 Hz, 2H), 6.79 (d, J=8.9 Hz, 2H), 6.06 (tt, J=56.4, 4.6 Hz, 1H), 4.26 (dtd, J=10.7, 5.2, 3.1 Hz, 1H), 3.97 (ddd, J=19.1, 10.6, 3.8 Hz, 2H), 3.83 (dd, J=9.8, 5.9 Hz, 1H), 3.55 (d, J=11.4 Hz, 1H), 3.45-3.32 (m, 2H), 1.99 (ddt, J=19.6, 15.8, 4.7 Hz, 2H), 1.47 (s, 3H).

### Example 435: 5-(4-(((2S,6S)-6-(2-fluoroethyl)-6-methyl-1,4-dioxan-2-yl)methoxy)phenyl)-2-oxo-6-(trifluoromethyl)-1,2-dihydropyridine-3-carboxamide (435)

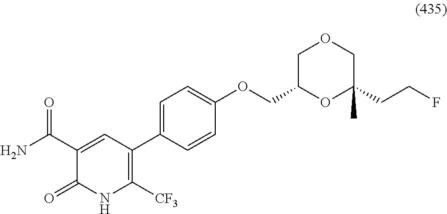 [SMILES: C[C@]1(CCF)COC[C@@H](COc2ccc(-c3cc(C(N)=O)c(=O)[nH]c3C(F)(F)F)cc2)O1]

5-(4-(((2S,6S)-6-(2-fluoroethyl)-6-methyl-1,4-dioxan-2-yl)methoxy)phenyl)-2-oxo-6-(trifluoromethyl)-1,2-dihydropyridine-3-carboxamide (435) was obtained using the method described for Example 405, except (2S,6S)-6-((4-bromophenoxy)methyl)-2-(methoxymethyl)-2-methyl-1,4-dioxane was replaced with (2S,6S)-6-((4-bromophenoxy)methyl)-2-(2-fluoroethyl)-2-methyl-1,4-dioxane. ¹H NMR (500 MHz, DMSO-d₆) δ 13.65 (s, 1H), 8.47 (s, 1H), 8.29 (s, 1H), 8.19 (s, 1H), 7.28 (d, J=8.6 Hz, 2H), 7.05 (d, J=8.6 Hz, 2H), 4.64 (t, J=6.1 Hz, 1H), 4.55 (t, J=6.1 Hz, 1H), 4.19 (dq, J=10.6, 4.6 Hz, 1H), 4.03-3.95 (m, 2H), 3.90 (dd, J=11.3, 3.0 Hz, 1H), 3.53 (d, J=11.3 Hz, 1H), 3.29 (dt, J=11.1, 5.4 Hz, 2H), 1.92-1.72 (m, 2H), 1.34 (s, 3H). LCMS Rt=4.01 min, m/z=459.0 (M+H).

### (2S,6S)-6-((4-bromophenoxy)methyl)-2-(2-fluoroethyl)-2-methyl-1,4-dioxane was Obtained Using the Following Procedure

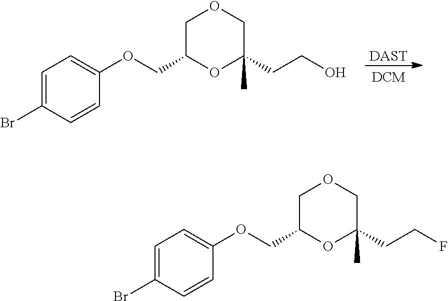

To a stirring solution of 2-((2S,6S)-6-((4-bromophenoxy)methyl)-2-methyl-1,4-dioxan-2-ylethan-1-ol (54 mg, 0.162 mmol) in dry DCM (1 mL) was added DAST (0.043 mL, 0.324 mmol) and the mixture stirred at RT for 1 hour. The reaction was quenched with sat. NaHCO₃ and stirred until gas evolution ceased. The mixture was concentrated and purified via silica gel chromatography (ISCO, 0 to 25% ethyl acetate/heptane) to give (2S,6S)-6-((4-bromophenoxy)methyl)-2-(2-fluoroethyl)-2-methyl-1,4-dioxane. LCMS Rt=0.84 min, m/z=313.2, 315.2 (M−HF).

### Example 436: 5-(4-(((2S,6R)-6-((2-fluoroethoxy)methyl)-6-methyl-1,4-dioxan-2-yl)methoxy)phenyl)-2-oxo-6-(trifluoromethyl)-1,2-dihydropyridine-3-carboxamide (436)

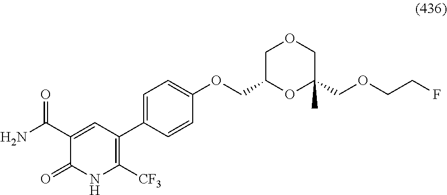 [SMILES: C[C@]1(COCCF)COC[C@@H](COc2ccc(-c3cc(C(N)=O)c(=O)[nH]c3C(F)(F)F)cc2)O1]

5-(4-(((2S,6R)-6-((2-fluoroethoxy)methyl)-6-methyl-1,4-dioxan-2-yl)methoxy)phenyl)-2-oxo-6-(trifluoromethyl)-1,2-dihydropyridine-3-carboxamide (436) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-((2-fluoroethoxy)methyl)-2-methyl-1,4-dioxane. ¹H NMR (500 MHz, DMSO-d6) δ 8.47 (s, 1H), 8.29 (s, 1H), 8.19 (s, 1H), 7.28 (d, J=8.7 Hz, 2H), 7.05 (d, J=8.8 Hz, 2H), 4.58 (t, J=4.0 Hz, 1H), 4.49 (t, J=4.0 Hz, 1H), 4.20 (dtd, J=10.4, 4.7, 2.8 Hz, 1H), 4.02-3.93 (m, 2H), 3.90 (dd, J=11.3, 2.9 Hz, 1H), 3.79-3.61 (m, 2H), 3.55 (d, J=11.3 Hz, 1H), 3.42-3.38 (m, 3H), 3.33-3.27 (m, 1H), 1.32 (s, 3H). LCMS Rt=3.21 min, m/z=489.3 (M+H).

### (2R,6S)-6-((4-bromophenoxy)methyl)-2-((2-fluoroethoxy)methyl)-2-methyl-1,4-dioxane was Obtained Using the Following Procedure

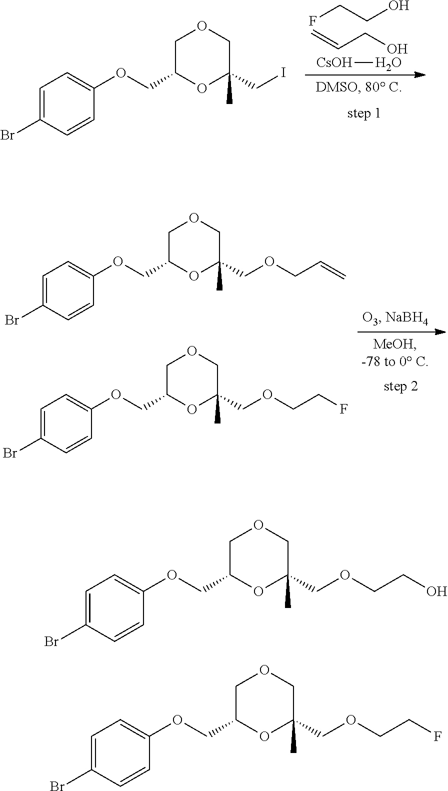

Step 1: To a microwave vial was added (2R,6S)-6-((4-bromophenoxy)methyl)-2-(iodomethyl)-2-methyl-1,4-dioxane (100 mg, 0.234 mmol) in dry DMSO (1 mL), followed by cesium hydroxide monohydrate (117 mg, 0.702 mmol) and 2-fluoroethanol (0.069 mL, 1.17 mmol). The reaction was then heated to 80° C. for 1 hour. Then allyl alcohol (0.191 mL, 2.81 mmol) and additional cesium hydroxide monohydrate (235 mg, 1.40 mmol) were added and the reaction continued at 80° C. for 1 hour. Additional allyl alcohol (0.096 mL, 1.40 mmol) and cesium hydroxide monohydrate (117 mg, 0.702 mmol) were added, and the reaction continued at 80° C. for 5 hours. The reaction was diluted with water, extracted with ether and washed with brine, dried over sodium sulfate and concentrated to give a crude mixture of (2S,6S)-2-((allyloxy)methyl)-6-((4-bromophenoxy)methyl)-2-methyl-1,4-dioxane and (2R,6S)-6-((4-bromophenoxy)methyl)-2-((2-fluoroethoxy)methyl)-2-methy-1,4-dioxane that was used directly in the next step without further purification. LCMS Rt=0.68 min, m/z=353.3, 355.3 (M+H) for (2S,6S)-2-((allyloxy)methyl)-6-((4-bromophenoxy)methyl)-2-methyl-1,4-dioxane; LCMS Rt=0.82 min, m/z=363.3, 365.3 (M+H) for (2R,6S)-6-((4-bromophenoxy)methyl)-2-((2-fluoroethoxy)methyl)-2-methyl-1,4-dioxane.

Step 2: A 50 mL flask was charged with the crude mixture of (2S,6S)-2-((allyloxy)methyl))-6-((4-bromophenoxy)methyl)-2-methyl-1,4-dioxane and (2R,6S)-6-((4-bromophenoxy)methyl)-2-((2-fluoroethoxy)methyl)-2-methyl-1,4-dioxane (84 mg) and dry MeOH (2 mL) and cooled to −78° C. Ozone was then bubbled through for 10 minutes at −78° C. Ozone generation was stopped, O2 was bubbled through for a minute, followed by bubbling with N₂ for another minute. The flask was warmed to 0° C., and sodium borohydride (36 mg, 0.951 mmol) was added in one portion, and the reaction stirred at 0° C. for 10 min. The crude was purified via silica gel chromatography (ISCO, 0 to 60% ethyl acetate/heptane) to give 2-(((2S,6S)-6-((4-bromophenoxy)methyl)-2-methyl-1,4-dioxan-2-yl)methoxy)ethan-1-ol and (2R,6S)-6-((4-bromophenoxy)methyl)-2-((2-fluoroethoxy)methyl)-2-methyl-1,4-dioxane. LCMS Rt=0.70 min, m/z=361.2, 363.2 (M+H) for 2-(((2S,6S)-6-((4-bromophenoxy)methyl)-2-methyl-1,4-dioxan-2-yl)methoxy)ethan-1-ol; LCMS Rt=0.82 min, m/z=363.2, 365.2 (M+H) for (2R,6S)-6-((4-bromophenoxy)methyl)-2-((2-fluoroethoxy)methyl)-2-methyl-1,4-dioxane.

(2R,6S)-6-((4-bromophenoxy)methyl)-2-((2-fluoroethoxy)methyl)-2-methyl-1,4-dioxane was also obtained using the method for the synthesis of (2S,6S)-6-((4-bromophenoxy)methyl)-2-(2-fluoroethyl)-2-methyl-1,4-dioxane, except 2-((2S,6S)-6-((4-bromophenoxy)methyl)-2-methyl-1,4-dioxan-2-yl)ethan-1-ol was replaced with 2-(((2S,6S)-6-((4-bromophenoxy)methyl)-2-methyl-1,4-dioxan-2-yl)methoxy)ethan-1-ol. LCMS Rt=0.82 min, m/z=363.2, 365.2 (M+H).

### Example 437: 5-(4-(((2S,6R)-6-cyclopropyl-6-methyl-1,4-dioxan-2-yl)methoxy)phenyl)-2-oxo-6-(trifluoromethyl)-1,2-dihydropyridine-3-carboxamide (437)

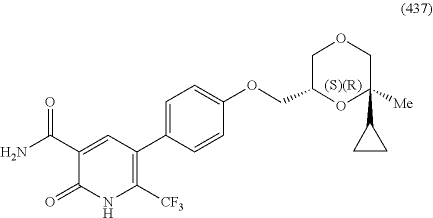 [SMILES: C[C@@]1(C2CC2)COC[C@@H](COc2ccc(-c3cc(C(N)=O)c(=O)[nH]c3C(F)(F)F)cc2)O1]

5-(4-(((2S,6R)-6-cyclopropyl-6-methyl-1,4-dioxan-2-yl)methoxy)phenyl)-2-oxo-6-(trifluoromethyl)-1,2-dihydropyridine-3-carboxamide (437) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-cyclopropyl-2-methyl-1,4-dioxane. ¹H NMR (400 MHz, DMSO-d6) δ=9.00 (br s, 1H), 8.15 (br s, 1H), 7.72 (m, 1H), 7.23 (d, J=8.0 Hz, 2H), 7.01 (d, J=8.4 Hz, 2H), 4.45 (m, 1H), 3.97 (m, 3H), 3.54 (m, 1H), 3.23 (m, 2H), 1.45 (m, 1H), 0.80 (s, 3H), 0.63 (m, 1H), 0.54 (m, 1H), 0.46 (m, 1H), 0.30 (m, 1H). LCMS: Rt=0.91 min, m/z=453.1 (M+H). Chiral HPLC: Rt=3.12 min, ee value=100%.

### (2R,6S)-6-((4-bromophenoxy)methyl)-2-cyclopropyl-2-methyl-1,4-dioxane was Obtained Using the Following Procedure

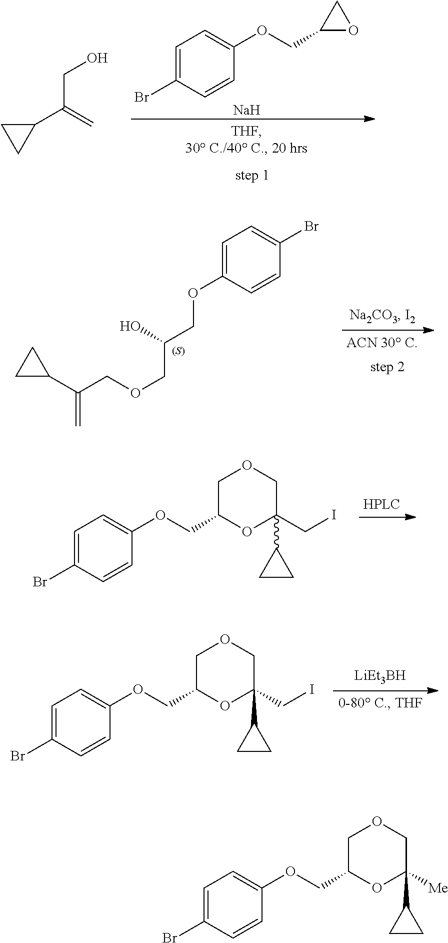

Step 1: A solution of 2-cyclopropylprop-2-en-1-ol (900 mg, 9.17 mmol) in dry THF (2 mL) was added to a suspension of NaH (459 mg, 11.48 mmol, 60% of purity) in dry THF (5 mL) at 30° C., and the mixture stirred at 30° C. for 1 hr. Then a solution of (S)-2-((4-bromophenoxy)methyl)oxirane (1.05 g, 4.59 mmol) in dry THF (5 mL) was added and the mixture stirred at 40° C. for 19 hrs. The reaction was quenched with water (100 mL) and the mixture extracted with EA (50 mL×3). The organic layers were combined, dried over anhydrous Na₂SO₄, filtered and concentrated to give a residue which was purified by Prep-HPLC to give (S)-1-(4-bromophenoxy)-3-((2-cyclopropylallyl)oxy)propan-2-ol. LCMS: Rt.=0.88 min, (M+H)=327.0/328.9. ¹H NMR (400 MHz, CDCl₃) δ=7.39-7.35 (m, 2H), 6.82-6.79 (m, 2H), 4.90-4.79 (m, 2H), 4.19 (m, 1H), 4.02-4.00 (m, 4H), 3.61-3.58 (m, 2H), 1.40 (m, 1H), 0.68-0.66 (i, 2H), 0.50-0.48 (i, 2H).

Step 2: To a solution of (S)-1-(4-bromophenoxy)-3-((2-cyclopropylallyl)oxy)propan-2-ol (700 mg, 2.14 mmol), NaHCO₃ (539 mg, 6.42 mmol, in MeCN (7 mL) was added I₂ (1.6 g, 6.42 mmol). Then the mixture was stirred at 30° C. for 16 hrs. The reaction mixture was quenched with saturated aq·Na₂SO₃ (100 mL) and extracted with EA (50 mL×2). The combined organic layers were dried over anhydrous Na₂SO₄, filtered and concentrated to give crude product which was purified by Prep-HPLC to give (2R,6S)-6-((4-bromophenoxy)methyl)-2-cyclopropyl-2-(iodomethyl)-1,4-dioxane (Peak 1) and (2S,6S)-((4-bromophenoxy)methyl)-2-cyclopropyl-2-(iodomethyl)-1,4-dioxane (Peak 2).

Peak 1: LCMS: Rt.=1.26 min, (M+H)=453.0/454.9. ¹H NMR (400 MHz, CDCl₃) δ=7.39 (m, 2H), 6.83 (m, 2H), 4.54 (m, 1H), 4.02 (m, 1H), 3.93 (m, 2H), 3.51 (m, 3H), 3.19 (m, 1H), 3.08 (m, 1H), 1.16 (m, 1H), 1.06 (m, 1H), 0.74 (m, 1H), 0.60 (m, 1H), 0.23 (m, 1H).

Peak 2: LCMS: Rt.=1.29 min, (M+H)=453.0/454.9. ¹H NMR (400 MHz, CDCl₃) δ=7.39 (m, 2H), 6.79 (m, 2H), 4.07 (m, 1H), 3.95 (m, 3H), 3.81 (m, 2H), 3.75 (m, 1H), 3.61 (m, 1H), 3.41 (m, 1H), 3.28 (m, 1H), 0.90 (m, 1H), 0.68 (m, 1H), 0.45 (m, 2H), 0.32 (m, 1H).

Step 3: A solution of lithium triethylborohydride in THF (1.0 M, 2.43 ml, 2.43 mmol) was added drop-wise to a solution of (2R,6S)-6-((4-bromophenoxy)methyl)-2-cyclopropyl-2-(iodomethyl)-1,4-dioxane (220 mg, 0.486 mmol) in THF (5.6 mL) for 0.5 hrs at 0° C. The ice bath was removed at the completion of the addition, and the reaction mixture was allowed to warm to room temperature over 20 minutes. The mixture was then heated at 80° C. for 1 hr and allowed to cool to room temperature. After careful addition of saturated aqueous sodium bicarbonate solution (20 mL), the mixture was extracted with EA (3×20 mL). The combined organic layers were dried over sodium sulfate, filtered, and concentrated in vacuo. The residue was purified by Prep-TLC (PE:EA=4:1) to give (2R,6S)-6-((4-bromophenoxy)methyl)-2-cyclopropyl-2-methyl-1,4-dioxane. TLC:PE:EA=5:1, Rf=0.7. LCMS: Rt=1.11 min, m/z=327.0 (M+H).

### Example 438: 5-(4-(((2S,6R)-6-cyclopropyl-6-(methoxymethyl)-1,4-dioxan-2-yl)methoxy)phenyl)-2-oxo-6-(trifluoromethyl)-1,2-dihydropyridine-3-carboxamide (438)

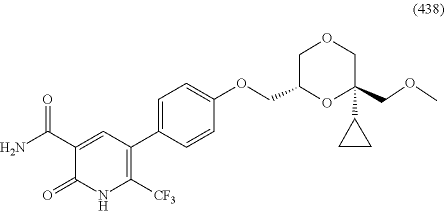 [SMILES: COC[C@]1(C2CC2)COC[C@@H](COc2ccc(-c3cc(C(N)=O)c(=O)[nH]c3C(F)(F)F)cc2)O1]

5-(4-(((2S,6R)-6-cyclopropyl-6-(methoxymethyl)-1,4-dioxan-2-yl)methoxy)phenyl)-2-oxo-6-(trifluoromethyl)-1,2-dihydropyridine-3-carboxamide (438) was obtained using the method described for Example 405, except (2S,6S)-6-((4-bromophenoxy)methyl)-2-(methoxymethyl)-2-methyl-1,4-dioxane was replaced with (2R,6S)-6-((4-bromophenoxy)methyl)-2-cyclopropyl-2-(methoxymethyl)-1,4-dioxane. ¹H NMR (400 MHz, DMSO-d6) δ=8.47 (br s, 1H), 7.96 (s, 1H), 7.79 (br s, 1H), 7.00 (d, J=8.4 Hz, 2H), 6.76 (d, J=8.4 Hz, 2H), 3.85 (m, 1H), 3.67 (m, 2H), 3.61 (m, 1H), 3.55 (m, 2H), 3.08 (s, 3H), 3.06 (m, 2H), 2.93 (m, 1H), 0.68 (m, 1H), 0.17 (m, 2H), 0.02 (m, 2H). LCMS: Rt=0.92 min, m/z=483.2 (M+H). Chiral HPLC: Rt=2.94 min, de value=100%.

### (2R,6S)-6-((4-bromophenoxy)methyl)-2-cyclopropyl-2-(methoxymethyl)-1,4-dioxane was Obtained Using the Following Procedure

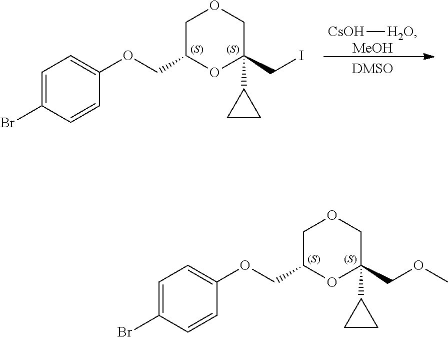

To a solution of (2S,6S)-6-((4-bromophenoxy)methyl)-2-cyclopropyl-2-(iodomethyl)-1,4-dioxane (300 mg, 0.66 mmol) in DMSO (3 mL) was added MeOH (106 mg, 3.30 mmol) and CsOH—H₂O (337 g, 1.98 mmol) at 25° C. The reaction mixture was stirred at 80° C. for 2 hrs. The combined mixture was washed with H₂O (10 ml) and extracted with EA (10 mL×2). The organic layer were combined, washed with brine (10 mL×2), dried over anhydrous Na2SO4, filtered and concentrated. The residue was purified by column chromatography (PE/EA=10/1) to give (2R,6S)-6-((4-bromophenoxy)methyl)-2-cyclopropyl-2-(methoxymethyl)-1,4-dioxane. LCMS: Rt=0.897, 0.924 min, m/z=357.0, 359.0 (M+H).

### Example 439: 5-(4-(((2S,6S)-6-cyclopropyl-6-(methoxymethyl)-1,4-dioxan-2-yl)methoxy)phenyl)-2-oxo-6-(trifluoromethyl)-1,2-dihydropyridine-3-carboxamide (439)

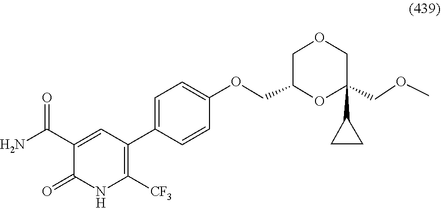 [SMILES: COC[C@@]1(C2CC2)COC[C@@H](COc2ccc(-c3cc(C(N)=O)c(=O)[nH]c3C(F)(F)F)cc2)O1]

5-(4-(((2S,6S)-6-cyclopropyl-6-(methoxymethyl)-1,4-dioxan-2-yl)methoxy)phenyl)-2-oxo-6-(trifluoromethyl)-1,2-dihydropyridine-3-carboxamide (439) was obtained using the method described for Example 405, except (2S,6S)-6-((4-bromophenoxy)methyl)-2-(methoxymethyl)-2-methyl-1,4-dioxane was replaced with (2S,6S)-6-((4-bromophenoxy)methyl)-2-cyclopropyl-2-(methoxymethyl)-1,4-dioxane. ¹H NMR (400 MHz, DMSO-d6) δ=8.86 (br s, 1H), 8.20 (s, 1H), 7.98 (br s, 1H), 7.25 (d, J=8.4 Hz, 2H), 7.03 (d, J=8.4 Hz, 2H), 4.45 (m, 1H), 3.99 (m, 2H), 3.43 (m, 1H), 3.39 (m, 2H), 3.13 (s, 3H), 3.10 (m, 2H), 1.07 (m, 1H), 0.80 (m, 1H), 0.57 (m, 1H), 0.43 (m, 1H), 0.33 (m, 1H), 0.31 (m, 1H). LCMS: Rt=0.90 min, m/z=483.3 (M+H). Chiral HPLC: Rt=3.26 min, de value=98.94%.

(2S,6S)-6-((4-bromophenoxy)methyl)-2-cyclopropyl-2-(methoxymethyl)-1,4-dioxane was obtained using the procedure for the synthesis of (2R,6S)-6-((4-bromophenoxy)methyl)-2-cyclopropyl-2-(methoxymethyl)-1,4-dioxane, except (2S,6S)-6-((4-bromophenoxy)methyl)-2-cyclopropyl-2-(iodomethyl)-1,4-dioxane was replaced with (2R,6S)-6-((4-bromophenoxy)methyl)-2-cyclopropyl-2-(iodomethyl)-1,4-dioxane.

### Example 440: 5-(4-(((5R,7S)-2,6,9-trioxaspiro[4.5]decan-7-yl)methoxy)phenyl)-2-oxo-6-(trifluoromethyl)-1,2-dihydropyridine-3-carboxamide (440)

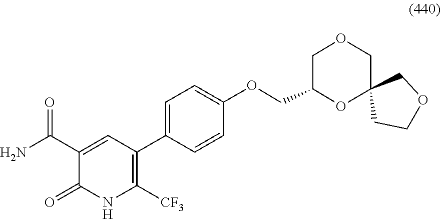 [SMILES: NC(=O)c1cc(-c2ccc(OC[C@@H]3COC[C@]4(CCOC4)O3)cc2)c(C(F)(F)F)[nH]c1=O]

5-(4-(((5R,7S)-2,6,9-trioxaspiro[4.5]decan-7-yl)methoxy)phenyl)-2-oxo-6-(trifluoromethyl)-1,2-dihydropyridine-3-carboxamide (440) was obtained after chiral SFC separation of the product obtained using the method described for Example 405, except (2S,6S)-6-((4-bromophenoxy)methyl)-2-(methoxymethyl)-2-methyl-1,4-dioxane was replaced with (7S)-7-((4-bromophenoxy)methyl)-2,6,9-trioxaspiro[4.5]decane. ¹H NMR: (400 MHz, DMSO-d6), δ=8.88 (s, 1H), 8.19 (s, 1H), 7.97 (s, 1H), 7.24 (d, J=8.4 Hz, 2H), 7.02 (d, J=8.4 Hz, 2H), 4.07 (m, 3H), 3.94 (m, 2H), 3.84 (m, 3H), 3.68 (m, 1H), 3.48 (m, 2H), 1.89-1.70 (m, 2H). LCMS: Rt=0.84 min, m/z=455 (M+H). Chiral HPLC: Rt=1.67 min, de value=100%, AS-3-MeOH(DEA)-5-40-3 mL-35T.

### (7S)-7-((4-bromophenoxy)methyl)-2,6,9-trioxaspiro[4.5]decane was Obtained Using the Following Procedure

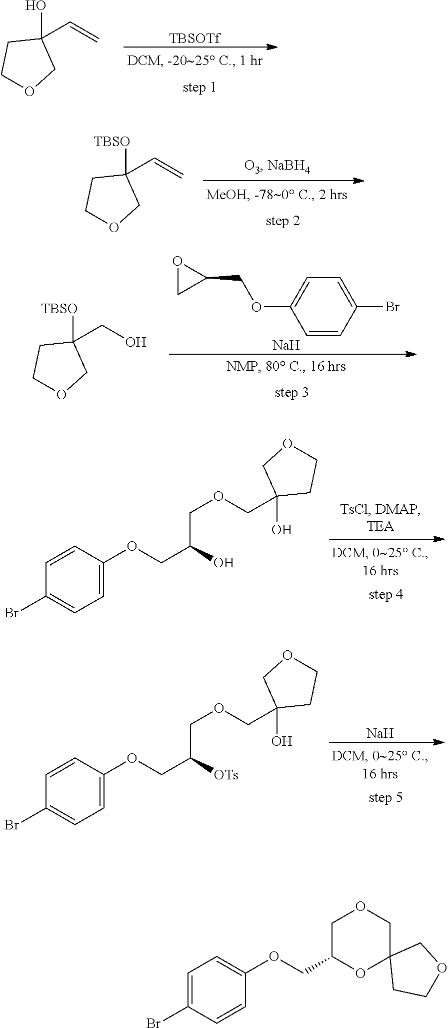

Step 1: To a solution of 3-vinyltetrahydrofuran-3-ol (10 g, 88 mmol) and 2,6-lutidine (28 g, 264 mmol) in DCM (100 mL) was added TBSOTf (46 g, 176 mmol) under N₂ at −20° C. Then the mixture was stirred at 25° C. for 1 hr. The reaction mixture was poured into water (200 mL). The resulting mixture was extracted with DCM (200 mL×2). The combined organic layers were dried over anhydrous Na₂SO₄, filtered and concentrated to give crude product which was purified by column chromatography (PE:EA=10:1) to give tert-butyldimethyl((3-vinyltetrahydrofuran-3-yl)oxy)silane. ¹H NMR (400 MHz, CDCl₃) δ=6.01 (m, 1H), 5.32 (m, 1H), 5.14 (m, 1H), 4.08 (m, 1H), 3.99 (m, 1H), 3.82 (m, 1H), 3.72 (m, 1H), 2.13 (m, 2H), 0.91 (s, 9H), 0.11 (m, 6H).

Step 2: To a solution of tert-butyldimethyl((3-vinyltetrahydrofuran-3-yl)oxy)silane (18 g, 78.9 mmol) in MeOH (10 mL) was stirred at −78° C. under 03 (15 psi) for 1 hr. The mixture was warmed at 0° C. then added NaBH4 (3.6 mg, 94.7 mmol) and stirred for 1 hr. The reaction mixture was concentrated to give crude product which was purified by column chromatography (PE:EA=10:1˜5:1) to give (3-((tert-butyldimethylsilyl)oxy)tetrahydrofuran-3-yl)methanol. ¹H NMR (400 MHz, CDCl₃) δ=4.05 (m, 1H), 3.90 (m, 1H), 3.77 (m, 1H), 3.67 (s, 1H), 2.74 (s, 1H), 2.00 (m, 2H), 0.93 (s, 10H), 0.10 (s, 6H).

Step 3: NaH (484 mg, 11.6 mmol) was added to a solution of (3-((tert-butyldimethylsilyl)oxy)tetrahydrofuran-3-yl)methanol (4.5 g, 19.4 mmol) in NMP (50 mL) at 0° C. and the mixture stirred at 0° C. for 1 hr. (R)-2-((4-bromophenoxy) methyl)oxirane (2.2 g, 9.7 mmol) was added and the mixture stirred at 80° C. for 16 hrs. The reaction mixture was poured into water (50 mL) and the resulting mixture extracted with EA (50 mL×2). The combined organic layers were dried over anhydrous Na₂SO₄, concentrated to give crude product which was purified by reverse phase column (FA) to give 3-(((R)-3-(4-bromophenoxy)-2-hydroxypropoxy)methyl)tetrahydrofuran-3-ol. LCMS: Rt=0.69 min, m/z=347.0, 349.0 (M+H).

Step 4: TsCl (1.3 g, 6.9 mmol) was added to a solution of 3-(((R)-3-(4-bromophenoxy)-2-hydroxypropoxy)methyl)tetrahydrofuran-3-ol (1.2 g, 3.5 mmol), TEA (1.0 g, 10.4 mmol), DMAP (210 mg, 1.7 mmol) in DCM (12 mL) at 0° C. and the mixture stirred at 25° C. for 72 hrs. The reaction mixture was poured into water (20 mL) and the resulting mixture extracted with DCM (20 mL×2). The combined organic layers were dried over anhydrous Na₂SO₄, filtered and concentrated to give crude product which was purified by reverse phase column (FA) to give (2R)-1-(4-bromophenoxy)-3-((3-hydroxytetrahydrofuran-3-yl)methoxy)propan-2-yl 4-methylbenzenesulfonate. ¹H NMR (400 MHz, CDCl₃) δ=7.82 (m, 2H), 7.39 (m, 4H), 6.69 (m, 2H), 4.99 (m, 1H), 4.13 (m, 2H), 4.03 (m, 1H), 3.89 (m, 1H), 3.85 (m, 2H), 3.72 (m, 4H), 2.47 (s, 3H), 1.98 (m, 2H).

Step 5: NaH (15 mg, 0.36 mmol) was added to a solution of (2R)-1-(4-bromophenoxy)-3-((3-hydroxytetrahydrofuran-3-yl)methoxy)propan-2-yl 4-methylbenzenesulfonate (120 mg, 0.24 mmol) in DMF (1 mL) at 0° C. and the mixture stirred at 25° C. for 16 hrs. The reaction mixture was poured into water (5 mL) and the resulting mixture extracted with EA (5 mL×2). The combined organic layers were dried over anhydrous Na₂SO₄, filtered and concentrated to give crude product which was purified by reverse phase column to give (7S)-7-((4-bromophenoxy)methyl)-2,6,9-trioxaspiro[4.5]decane. ¹H NMR (400 MHz, CDCl₃) δ=7.28 (m, 2H), 6.70 (m, 2H), 3.97 (m, 6H), 2.13 (m, 1H), 1.72 (m, 1H), 1.60 (m, 1H).

### Biological Assays and Data

The activity of a compound disclosed herein may be assessed by the following in vitro and in vivo methods. Using the test assays described herein, provided compounds exhibit inhibitory efficacy in accordance with Table 1.

### Influenza Virus Neuraminidase Assay (NA Assay)

For influenza NA assays, MDCK cells were plated in Phenol Red-free DMEM (Gibco) supplemented with 2 mM L-Glutamine, 1% sodium pyruvate (Cellgro, Manassas, VA) and 0.1% BSA at cell densities of 1.8×10⁴ cells/well in 384-well format. Compounds were added to the cells 2 hours pre-infection. Infections were performed at MOI 0.005 and the plates were incubated at 37° C., 5% CO₂ for 48 hours. Following incubation, neuraminidase activity was evaluated with the NA assay kit (ThermoFisher, Carlsbad, CA). For cell toxicity measurement, CellTiter-Glo® (Promega, Madison, WI) was added to treated cells according to manufacturer's instructions.

### Influenza Virus Minigenome Assays (RNP Assay)

For influenza A virus minigenome reporter assays, 293T cells were transfected with expression vectors encoding PB2, PB1, PA, NP proteins and an influenza A Luciferase reporter plasmid. Cells were harvested in Dulbecco's modified Eagle's medium (DMEM) minus phenol red, supplemented with 10% heat inactivated FBS (fetal bovine serum), 1% sodium pyruvate and 1% L-glutamine (Cellgro, Manassas, VA). The five plasmids were co-transfected with Fugene 6 transfection reagent (Promega, Madison, WI) with a 1:3 ratio DNA (μg):Fugene 6 (μl), in OptiMEM® (Gibco, Carlsbad, CA). Transfections were performed at cell densities of 1.8×10⁴ cells/well in 384-well format. Compounds were added 2 hours post-transfection, and plates were incubated at 37° C., 5% CO₂ for 48 hours. Following incubation, cells were lysed and luciferase production quantified by addition of Britelite Plus® (Perkin-Elmer, Waltham, MA). For cell toxicity measurement, CellTiter-Glo® (Promega, Madison, WI) was added to treated cells following manufacturer's instructions.

TABLE 1
Activity of Selected Compounds on multiple
flu strains (NA assay except for Ex 19A).

| | | RNP_{—} | NA | RNP_{—} | NA | NA_{—} | RNP_{—} |
|---|---|---|---|---|---|---|---|
| Example | Synthesis | Alaska_{—} | Alaska | CAL_{—} | CAL | PR8 | Hubei |
| Number | Scheme | H3N2 | H3N2 | H1N1 | H1N1 | H1N1 | B |
| 348 | D | +++++ | nd | ++++ | nd | nd | +++ |
| 84 | C | ++++ | ++++ | ++ | ++ | nd | + |
| 164 | C | ++++++ | ++++++ | nd | +++++ | nd | nd |
| 104 | C | nd | +++++ | nd | +++++ | nd | nd |
| 30 | B | +++++ | +++++ | ++++ | +++++ | nd | +++ |
| 99 | C | +++++ | ++++++ | +++++ | +++++ | nd | + |
| 126 | C | +++++ | +++++ | ++++ | ++++ | nd | + |
| 127 | C | nd | ++++ | nd | ++++ | nd | nd |
| 128 | C | nd | ++++ | nd | + | nd | nd |
| 105 | C | nd | +++++ | nd | ++++ | nd | nd |
| 120 | C | nd | ++++++ | nd | +++++ | nd | nd |
| 106 | C | nd | +++++ | nd | ++++ | nd | nd |
| 97 | C | +++ | ++++ | ++ | ++++ | nd | + |
| 85 | C | ++++ | ++++ | + | + | nd | + |
| 150 | C | nd | ++++ | nd | + | nd | nd |
| 129 | C | nd | +++++ | nd | ++++ | nd | nd |
| 77 | C | ++++ | +++++ | ++ | +++ | nd | ++ |
| 107 | C | +++++ | +++++ | ++++ | ++++ | nd | + |
| 121 | C | +++++ | +++++ | nd | ++++ | nd | nd |
| 94 | C | +++++ | +++++ | +++++ | +++++ | nd | + |
| 130 | C | nd | ++++ | nd | ++++ | nd | nd |
| 78 | C | ++++ | +++++ | +++++ | ++++++ | nd | + |
| 11 | A | nd | +++++ | nd | ++++ | nd | nd |
| 79 | C | ++++ | +++++ | ++++ | +++++ | nd | +++ |
| 131 | C | nd | +++++ | nd | +++ | nd | nd |
| 132 | C | nd | ++++ | nd | +++ | nd | nd |
| 91 | C | ++++ | ++++ | + | + | nd | + |
| 133 | C | nd | ++++ | nd | ++ | nd | nd |
| 360 | D | +++++ | nd | +++++ | nd | nd | +++ |
| 134 | C | +++++ | +++++ | ++++ | ++++ | nd | + |
| 108 | C | nd | +++++ | nd | +++++ | nd | nd |
| 16 | A | nd | nd | + | nd | ++++ | + |
| 1 | A | +++++ | +++++ | ++++ | ++++ | +++++ | + |
| 5 | A | ++++ | nd | + | nd | +++ | + |
| 18 | A | nd | nd | ++ | nd | ++++ | ++ |
| 21 | A | nd | nd | nd | nd | +++ | nd |
| 24 | A | nd | nd | nd | nd | +++ | nd |
| 23 | A | nd | nd | nd | nd | +++ | nd |
| 26 | A | nd | nd | nd | nd | +++ | nd |
| 2 | A | +++++ | +++++ | + | ++ | +++++ | + |
| 25 | A | nd | nd | nd | nd | +++ | nd |
| 4 | A | ++++ | +++++ | ++++ | ++++ | +++++ | +++ |
| 17 | A | nd | nd | ++ | nd | ++++ | ++ |
| 14 | A | nd | nd | + | nd | ++++ | + |
| 8 | A | +++ | nd | + | nd | ++ | nd |
| 15 | A | nd | nd | + | nd | ++++ | + |
| 20 | A | nd | nd | + | nd | ++++ | + |
| 12 | A | nd | +++ | nd | + | ++ | nd |
| 22 | A | nd | nd | nd | nd | +++ | nd |
| 13 | A | nd | nd | + | nd | ++++ | + |
| 19 | A | nd | nd | nd | nd | ++++ | nd |
| 40 | B | ++++ | nd | + | nd | nd | + |
| 44 | B | ++++ | nd | ++ | nd | nd | + |
| 29 | B | +++++ | +++++ | +++ | ++++ | nd | ++ |
| 6 | A | ++++ | nd | +++ | nd | nd | ++++ |
| 38 | B | ++++ | nd | + | nd | nd | + |
| 27 | B | +++++ | +++++ | ++++ | ++++ | nd | ++ |
| 36 | B | ++++ | ++++ | ++ | +++ | nd | ++ |
| 42 | B | ++++ | ++++ | ++ | ++ | nd | ++ |
| 47 | B | +++ | nd | ++ | nd | nd | + |
| 35 | B | ++++ | nd | ++ | nd | nd | + |
| 31 | B | +++++ | nd | ++++ | nd | nd | ++++ |
| 46 | B | ++++ | nd | + | nd | nd | + |
| 45 | B | ++++ | nd | + | nd | nd | + |
| 34 | B | ++++ | nd | +++ | nd | nd | + |
| 49 | B | +++ | nd | + | nd | nd | + |
| 43 | B | ++++ | ++++ | ++ | ++ | nd | ++++ |
| 37 | B | ++++ | nd | + | nd | nd | + |
| 28 | B | +++++ | nd | ++++ | nd | nd | ++++ |
| 41 | B | ++++ | nd | + | nd | nd | + |
| 33 | B | ++++ | nd | ++++ | nd | nd | ++++ |
| 3 | A | +++++ | ++++ | + | + | nd | + |
| 54 | C | +++++ | +++++ | +++++ | +++++ | nd | ++ |
| 55 | C | ++ | nd | + | nd | nd | + |
| 56 | C | ++++ | +++++ | ++++ | ++++ | nd | + |
| 57 | C | +++++ | nd | ++++ | nd | nd | +++ |
| 58 | C | ++ | +++ | ++++ | ++++ | nd | +++ |
| 59 | C | ++++ | nd | ++ | nd | nd | +++ |
| 60 | C | ++++ | nd | + | nd | nd | + |
| 61 | C | ++++ | nd | +++ | nd | nd | nd |
| 62 | C | ++++ | nd | + | nd | nd | nd |
| 63 | C | ++++ | nd | +++ | nd | nd | nd |
| 64 | C | ++ | nd | + | nd | nd | nd |
| 65 | C | +++ | nd | + | nd | nd | nd |
| 66 | C | +++++ | +++++ | ++++ | +++++ | nd | +++++ |
| 67 | C | +++ | nd | + | nd | nd | nd |
| 68 | C | +++ | nd | + | nd | nd | nd |
| 69 | C | ++ | nd | + | nd | nd | nd |
| 70 | C | +++++ | +++++ | +++ | ++++ | nd | + |
| 71 | C | +++++ | +++++ | +++++ | ++++++ | nd | + |
| 72 | C | +++++ | ++++++ | ++++ | +++++ | nd | + |
| 73 | C | +++++ | +++++ | ++++ | +++++ | nd | + |
| 9 | A | +++ | nd | + | nd | nd | + |
| 74 | C | ++++ | +++++ | ++++ | +++++ | nd | ++ |
| 151 | C | nd | ++++ | nd | + | nd | nd |
| 178 | C | nd | ++++++ | nd | +++++ | nd | nd |
| 135 | C | nd | +++++ | nd | +++++ | nd | nd |
| 136 | C | nd | ++++ | nd | ++++ | nd | nd |
| 92 | C | ++++ | ++++ | ++ | +++ | nd | + |
| 86 | C | +++++ | +++++ | ++ | ++ | nd | +++ |
| 109 | C | ++++ | ++++ | ++++ | +++++ | nd | + |
| 80 | C | ++++ | +++++ | +++ | ++++ | nd | ++ |
| 137 | C | nd | ++++ | nd | + | nd | nd |
| 138 | C | nd | ++++ | nd | ++ | nd | nd |
| 139 | C | nd | +++++ | nd | ++++ | nd | nd |
| 140 | C | nd | ++++ | nd | +++ | nd | nd |
| 87 | C | ++++ | +++++ | +++ | +++ | nd | ++++ |
| 100 | C | ++++ | +++++ | +++++ | +++++ | nd | +++ |
| 93 | C | +++++ | +++++ | +++++ | +++++ | nd | ++ |
| 110 | C | +++++ | +++++ | ++++ | ++++ | nd | +++ |
| 111 | C | nd | +++++ | nd | ++++ | nd | nd |
| 10 | A | +++ | nd | + | nd | nd | + |
| 76 | C | ++++ | +++++ | ++++ | +++++ | nd | ++ |
| 124 | C | nd | +++++ | nd | +++++ | nd | nd |
| 103 | C | nd | ++++ | nd | + | nd | nd |
| 159 | C | ++++++ | ++++++ | +++++ | +++++ | nd | nd |
| 125 | C | +++++ | +++++ | ++++ | +++++ | nd | nd |
| 48 | B | +++ | +++ | ++ | ++ | nd | + |
| 122 | C | nd | +++++ | nd | ++ | nd | nd |
| 10 | C | +++ | ++++ | +++ | ++++ | nd | + |
| 14 | C | +++++ | ++++ | ++ | ++ | nd | nd |
| 39 | B | ++++ | +++++ | ++++ | +++++ | nd | ++ |
| 142 | C | nd | ++++ | nd | ++ | nd | nd |
| 112 | C | nd | +++++ | nd | ++++++ | nd | nd |
| 88 | C | +++ | +++ | + | + | nd | +++ |
| 143 | C | +++++ | +++++ | ++++ | ++++ | nd | ++ |
| 81 | C | +++++ | ++++++ | ++++ | +++++ | nd | ++ |
| 82 | C | ++++++ | +++++ | +++++ | +++++ | nd | + |
| 144 | C | nd | ++++ | nd | +++ | nd | nd |
| 95 | C | ++++ | +++++ | +++ | +++++ | nd | + |
| 75 | C | ++++ | nd | ++ | nd | nd | + |
| 145 | C | nd | +++ | nd | + | nd | nd |
| 102 | C | +++++ | ++++++ | ++++ | +++++ | nd | +++ |
| 7 | A | +++ | nd | + | nd | nd | + |
| 113 | C | nd | ++++ | nd | +++ | nd | nd |
| 89 | C | +++++ | +++++ | ++++ | +++++ | nd | + |
| 98 | C | +++++ | +++++ | +++++ | ++++++ | nd | ++++ |
| 146 | C | nd | +++++ | nd | +++++ | nd | nd |
| 147 | C | nd | ++++ | nd | ++ | nd | nd |
| 148 | C | nd | ++++ | nd | +++ | nd | nd |
| 114 | C | nd | +++++ | nd | ++++ | nd | nd |
| 90 | C | ++++ | ++++ | ++ | ++ | nd | + |
| 83 | C | ++++ | nd | ++ | nd | nd | + |
| 115 | C | ++++ | +++++ | ++ | ++++ | nd | nd |
| 116 | C | nd | ++++ | nd | +++++ | nd | nd |
| 117 | C | +++++ | +++++ | +++++ | ++++++ | nd | nd |
| 32 | B | +++++ | +++++ | ++++ | +++++ | nd | + |
| 118 | C | ++++ | ++++ | ++ | +++ | nd | nd |
| 149 | C | nd | ++++ | nd | ++ | nd | nd |
| 96 | C | +++++ | +++++ | ++++ | ++++ | nd | + |
| 389 | Z | +++++ | nd | ++++ | nd | nd | +++ |
| 208 | C | +++++ | nd | ++++ | nd | nd | +++ |
| 123 | C | +++++ | ++++++ | +++++ | +++++ | nd | nd |
| 357 | D | ++++++ | nd | +++++ | nd | nd | +++ |
| 119 | C | nd | +++++ | nd | +++++ | nd | nd |
| 207 | C | +++++ | +++++ | ++++ | +++++ | nd | +++ |
| 50 | Z | +++++ | +++++ | +++ | ++++ | nd | + |
| 51 | Z | +++++ | nd | ++ | nd | nd | +++ |
| 52 | Z | ++++ | nd | ++ | nd | nd | nd |
| 152 | C | nd | +++ | nd | ++ | nd | nd |
| 153 | C | nd | +++++ | nd | ++++ | nd | nd |
| 154 | C | nd | +++++ | nd | +++ | nd | nd |
| 155 | C | nd | +++++ | nd | ++++ | nd | nd |
| 156 | C | nd | ++++ | nd | + | nd | nd |
| 157 | C | nd | +++++ | nd | +++ | nd | nd |
| 158 | C | nd | ++++++ | nd | ++++ | nd | nd |
| 160 | C | nd | +++++ | nd | ++ | nd | nd |
| 161 | C | ++++++ | ++++++ | +++++ | +++++ | nd | ++ |
| 162 | C | nd | +++++ | nd | ++ | nd | nd |
| 163 | C | nd | +++++ | nd | ++++ | nd | nd |
| 165 | C | nd | +++++ | nd | ++++ | nd | nd |
| 166 | C | nd | +++++ | nd | ++++ | nd | nd |
| 167 | C | +++++ | nd | +++ | nd | nd | nd |
| 168 | C | +++++ | nd | ++++ | nd | nd | nd |
| 169 | C | +++++ | nd | + | nd | nd | nd |
| 170 | C | +++++ | nd | ++++ | nd | nd | nd |
| 171 | C | +++++ | nd | ++++ | nd | nd | nd |
| 172 | C | ++++++ | nd | +++++ | nd | nd | nd |
| 173 | C | +++++ | nd | ++++ | nd | nd | nd |
| 174 | C | nd | +++++ | nd | ++++ | nd | nd |
| 175 | C | +++++ | nd | ++++ | nd | nd | + |
| 176 | C | ++++++ | nd | ++++ | nd | nd | + |
| 177 | Z | +++++ | +++++ | ++++ | ++++ | nd | + |
| 258 | C | nd | ++++ | nd | + | nd | nd |
| 179 | C | nd | +++++ | nd | ++++ | nd | nd |
| 180 | C | nd | +++++ | ++++ | ++++ | nd | nd |
| 181 | C | ++++ | +++ | + | ++ | nd | + |
| 182 | C | +++++ | +++++ | ++++ | ++++ | nd | nd |
| 183 | C | ++++ | nd | ++ | nd | nd | nd |
| 184 | C | +++++ | nd | ++++ | nd | nd | ++ |
| 185 | C | ++++ | nd | ++++ | nd | nd | ++ |
| 186 | C | ++++ | ++++ | ++++ | ++++ | nd | + |
| 187 | C | ++++ | +++++ | +++ | ++++ | nd | nd |
| 188 | C | +++ | ++++ | + | + | nd | nd |
| 189 | C | ++++ | ++++ | ++ | ++ | nd | + |
| 190 | C | +++++ | +++++ | ++ | +++ | nd | nd |
| 191 | C | +++++ | +++++ | ++ | + | nd | + |
| 192 | C | +++++ | +++++ | +++ | ++ | nd | + |
| 193 | C | ++++ | +++++ | ++ | ++ | nd | + |
| 194 | C | +++++ | +++++ | +++ | ++ | nd | + |
| 195 | C | ++++ | +++ | + | + | nd | + |
| 196 | C | +++++ | +++++ | ++ | ++ | nd | + |
| 197 | C | +++++ | nd | ++ | nd | nd | nd |
| 198 | C | +++++ | nd | +++ | nd | nd | + |
| 199 | C | nd | ++++ | nd | + | nd | nd |
| 200 | C | nd | ++++ | nd | + | nd | nd |
| 201 | C | nd | ++++ | nd | ++ | nd | nd |
| 202 | C | +++++ | nd | ++++ | nd | nd | ++ |
| 203 | C | +++++ | nd | +++ | nd | nd | + |
| 204 | C | ++++ | +++ | + | + | nd | nd |
| 205 | C | ++++ | ++++ | ++ | ++ | nd | nd |
| 206 | C | +++++ | +++++ | +++ | +++ | nd | + |
| 317 | C | +++++ | nd | +++++ | nd | nd | +++ |
| 209 | C | +++++ | nd | ++++ | nd | nd | +++ |
| 210 | C | +++++ | nd | +++ | nd | nd | +++ |
| 211 | C | +++++ | nd | ++++ | nd | nd | ++++ |
| 212 | C | nd | ++++++ | nd | +++++ | nd | nd |
| 213 | C | +++++ | ++++++ | +++++ | +++++ | nd | nd |
| 214 | C | +++++ | nd | +++ | nd | nd | nd |
| 215 | C | nd | +++++ | nd | +++ | nd | nd |
| 216 | C | +++++ | ++++++ | +++++ | +++++ | nd | nd |
| 217 | C | +++++ | nd | +++ | nd | nd | + |
| 218 | C | +++++ | nd | +++ | nd | nd | + |
| 219 | C | +++++ | nd | +++ | nd | nd | + |
| 220 | C | +++++ | nd | +++ | nd | nd | + |
| 221 | C | +++++ | nd | ++++ | nd | nd | ++ |
| 222 | C | +++++ | nd | +++ | nd | nd | + |
| 223 | C | +++++ | +++++ | ++++ | ++++ | nd | + |
| 224 | C | +++++ | +++++ | +++ | +++ | nd | + |
| 225 | C | +++++ | +++++ | ++++ | ++++ | nd | + |
| 226 | C | +++++ | +++++ | ++++ | ++++ | nd | + |
| 227 | C | +++++ | +++++ | ++++ | ++++ | nd | + |
| 228 | C | +++++ | +++++ | ++++ | ++++ | nd | ++ |
| 229 | C | +++++ | ++++ | ++++ | +++ | nd | + |
| 230 | C | nd | ++++ | ++ | +++ | nd | nd |
| 231 | C | +++++ | +++++ | +++ | ++++ | nd | + |
| 232 | C | +++++ | +++++ | ++++ | +++++ | nd | ++ |
| 233 | C | +++ | nd | + | nd | nd | + |
| 234 | C | ++++ | nd | + | nd | nd | + |
| 235 | C | ++++ | nd | + | nd | nd | + |
| 236 | C | nd | +++++ | nd | +++++ | nd | +++ |
| 237 | C | +++++ | ++++++ | +++++ | +++++ | nd | + |
| 238 | C | +++++ | +++++ | +++++ | +++++ | nd | + |
| 239 | C | +++++ | +++++ | ++++ | ++++ | nd | ++ |
| 240 | C | ++++ | +++++ | +++ | +++ | nd | nd |
| 241 | C | ++++ | ++++ | + | + | nd | nd |
| 242 | C | +++++ | +++++ | +++ | ++++ | nd | nd |
| 244 | C | +++ | ++ | + | + | nd | nd |
| 245 | C | ++++ | +++++ | ++ | ++ | nd | nd |
| 243 | C | +++ | nd | +++ | nd | nd | ++++ |
| 246 | C | ++ | ++ | + | + | nd | nd |
| 247 | C | +++++ | +++++ | ++++ | ++++ | nd | + |
| 248 | C | +++++ | nd | ++++ | nd | nd | +++ |
| 249 | C | +++++ | nd | ++++ | nd | nd | +++ |
| 250 | C | ++++ | ++++ | nd | + | nd | + |
| 251 | C | ++++ | ++++ | + | + | nd | nd |
| 252 | C | ++++ | +++++ | ++ | ++++ | nd | nd |
| 253 | C | ++++ | +++++ | +++ | ++++ | nd | nd |
| 254 | C | nd | +++++ | nd | ++++ | nd | + |
| 255 | C | nd | +++++ | nd | +++ | nd | nd |
| 256 | C | nd | +++++ | nd | +++ | nd | Nd |
| 257 | C | ++++ | nd | ++ | nd | nd | + |
| 259 | C | nd | +++++ | nd | +++ | nd | nd |
| 260 | C | nd | ++++ | nd | + | nd | nd |
| 261 | C | +++++ | nd | ++++ | nd | nd | nd |
| 262 | C | +++++ | nd | +++ | nd | nd | + |
| 263 | C | +++++ | nd | ++++ | nd | nd | + |
| 264 | C | +++++ | nd | +++ | nd | nd | nd |
| 265 | C | +++++ | nd | ++++ | nd | nd | + |
| 266 | C | +++++ | nd | ++++ | nd | nd | + |
| 267 | C | +++++ | nd | +++ | nd | nd | + |
| 268 | C | +++++ | nd | ++ | nd | nd | + |
| 269 | C | +++++ | nd | ++++ | nd | nd | + |
| 270 | C | ++++ | nd | ++ | nd | nd | + |
| 271 | C | +++++ | nd | +++ | nd | nd | + |
| 272 | C | +++++ | nd | +++ | nd | nd | + |
| 273 | C | +++++ | nd | ++++ | nd | nd | ++ |
| 274 | C | +++++ | nd | ++++ | nd | nd | nd |
| 275 | C | ++++ | nd | +++ | nd | nd | nd |
| 276 | C | +++++ | nd | +++ | nd | nd | nd |
| 277 | C | ++++ | nd | +++ | nd | nd | nd |
| 278 | C | +++++ | nd | +++++ | nd | nd | nd |
| 279 | C | +++++ | nd | +++++ | nd | nd | nd |
| 280 | C | ++++ | nd | ++++ | nd | nd | nd |
| 281 | C | +++++ | nd | ++++ | nd | nd | nd |
| 282 | C | +++++ | nd | ++++ | nd | nd | nd |
| 283 | C | ++++++ | nd | +++++ | nd | nd | +++ |
| 284 | C | ++++++ | nd | +++++ | nd | nd | nd |
| 285 | C | ++++ | nd | ++ | nd | nd | nd |
| 286 | C | +++++ | nd | ++ | nd | nd | + |
| 287 | C | ++++ | nd | +++ | nd | nd | nd |
| 288 | C | +++++ | nd | ++ | nd | nd | nd |
| 289 | C | +++++ | nd | +++ | nd | nd | +++ |
| 290 | C | +++++ | nd | ++++ | nd | nd | +++ |
| 291 | C | +++++ | nd | +++ | nd | nd | +++ |
| 292 | C | +++++ | nd | +++ | nd | nd | +++ |
| 293 | C | +++++ | nd | ++++ | nd | nd | +++ |
| 294 | C | ++++ | nd | +++ | nd | nd | +++ |
| 295 | C | ++++ | nd | +++ | nd | nd | +++ |
| 296 | C | +++++ | nd | +++ | nd | nd | +++ |
| 297 | C | ++++ | nd | ++ | nd | nd | nd |
| 298 | C | ++++ | nd | ++ | nd | nd | nd |
| 299 | C | +++ | nd | ++ | nd | nd | nd |
| 300 | C | +++++ | nd | ++++ | nd | nd | ++ |
| 301 | C | +++ | nd | ++ | nd | nd | nd |
| 302 | C | ++++ | nd | ++ | nd | nd | + |
| 303 | C | ++++ | nd | +++ | nd | nd | ++ |
| 304 | C | +++++ | nd | +++ | nd | nd | nd |
| 305 | C | +++++ | nd | +++ | nd | nd | + |
| 306 | C | +++ | nd | ++ | nd | nd | +++ |
| 307 | C | ++++ | nd | +++ | nd | nd | nd |
| 308 | C | ++++ | nd | +++ | nd | nd | nd |
| 309 | C | +++ | nd | ++ | nd | nd | nd |
| 310 | C | ++++ | nd | ++ | nd | nd | nd |
| 311 | C | ++++ | nd | ++ | nd | nd | nd |
| 312 | C | +++++ | nd | ++++ | nd | nd | +++ |
| 313 | C | +++++ | nd | ++++ | nd | nd | ++ |
| 314 | C | +++++ | nd | ++++ | nd | nd | ++ |
| 315 | C | +++++ | nd | ++++ | nd | nd | ++ |
| 316 | C | +++++ | nd | +++++ | nd | nd | nd |
| 318 | C | +++++ | nd | ++++ | nd | nd | +++ |
| 319 | C | +++++ | nd | ++++ | nd | nd | +++ |
| 320 | C | +++++ | nd | ++++ | nd | nd | +++ |
| 321 | C | +++++ | nd | ++++ | nd | nd | +++ |
| 322 | C | +++++ | nd | ++++ | nd | nd | + |
| 323 | C | +++++ | nd | ++++ | nd | nd | ++ |
| 324 | C | +++++ | nd | ++++ | nd | nd | |
| 325 | C | +++++ | ++++++ | +++++ | +++++ | nd | ++ |
| 327 | C | ++++++ | nd | +++++ | nd | nd | +++ |
| 328 | C | +++++ | ++++++ | +++++ | ++++++ | nd | ++ |
| 329 | C | ++++++ | ++++++ | +++++ | +++++ | nd | + |
| 330 | C | ++++++ | ++++++ | +++++ | ++++++ | nd | + |
| 331 | C | +++++ | nd | ++++ | nd | nd | ++ |
| 332 | C | +++++ | nd | +++++ | nd | nd | nd |
| 333 | C | +++++ | nd | ++++ | nd | nd | ++++ |
| 335 | C | ++++++ | ++++++ | +++++ | ++++++ | nd | +++ |
| 334 | | nd | nd | nd | nd | nd | nd |
| 336 | Z | +++++ | nd | ++++ | nd | nd | +++ |
| 337 | Z | +++++ | nd | +++++ | nd | nd | +++ |
| 338 | C | +++++ | nd | ++++ | nd | nd | ++ |
| 339 | C | +++++ | nd | ++++ | nd | nd | nd |
| 340 | C | +++++ | nd | ++++ | nd | nd | +++ |
| 341 | C | +++++ | nd | ++++ | nd | nd | +++ |
| 342 | C | +++++ | nd | +++ | nd | nd | +++ |
| 343 | C | +++++ | nd | ++++ | nd | nd | +++ |
| 344 | C | +++++ | nd | ++++ | nd | nd | +++ |
| 345 | C | +++++ | nd | ++++ | nd | nd | +++ |
| 346 | C | +++++ | nd | +++ | nd | nd | +++ |
| 347 | C | +++++ | nd | +++ | nd | nd | +++ |
| 349 | D | ++++++ | nd | +++++ | nd | nd | +++ |
| 350 | | nd | nd | ns | nd | nd | nd |
| 351 | D | +++++ | ++++++ | +++++ | +++++ | nd | nd |
| 352 | D | +++++ | ++++++ | ++++ | +++++ | nd | nd |
| 353 | D | +++++ | nd | +++++ | nd | nd | +++ |
| 354 | D | +++++ | nd | ++++ | nd | nd | +++ |
| 355 | D | ++++ | nd | +++ | nd | nd | +++ |
| 356 | D | +++++ | nd | +++++ | nd | nd | +++ |
| 358 | D | +++++ | nd | +++++ | nd | nd | +++ |
| 359 | D | +++++ | nd | ++++ | nd | nd | +++ |
| 362 | D | +++++ | nd | +++++ | nd | nd | +++ |
| 361 | D | +++++ | nd | +++++ | nd | nd | +++ |
| 363 | D | +++++ | nd | +++++ | nd | nd | +++ |
| 364 | D | +++++ | nd | +++++ | nd | nd | +++ |
| 365 | D | +++++ | nd | +++++ | nd | nd | +++ |
| 366 | D | +++++ | nd | +++++ | nd | nd | +++ |
| 367 | D | +++++ | nd | +++++ | nd | nd | +++ |
| 368 | D | +++++ | nd | ++++ | nd | nd | +++ |
| 369 | D | +++++ | nd | ++++ | nd | nd | +++ |
| 370 | D | +++++ | nd | ++++ | nd | nd | +++ |
| 371 | D | +++++ | nd | ++++ | nd | nd | +++ |
| 372 | D | +++++ | nd | +++++ | nd | nd | ++++ |
| 373 | D | +++++ | nd | ++++ | nd | nd | +++ |
| 374 | D | +++++ | nd | ++++ | nd | nd | +++ |
| 375 | D | +++++ | nd | ++++ | nd | nd | +++ |
| 376 | D | ++++ | nd | +++ | nd | nd | +++ |
| 377 | D | +++++ | nd | +++ | nd | nd | +++ |
| 378 | D | +++++ | nd | ++++ | nd | nd | +++ |
| 379 | D | +++++ | nd | ++++ | nd | nd | +++ |
| 380 | Z | ++++ | nd | +++ | nd | nd | +++ |
| 381 | Z | +++++ | nd | +++ | nd | nd | +++ |
| 382 | D | +++++ | nd | +++++ | nd | nd | +++ |
| 383 | | nd | nd | nd | nd | nd | nd |
| 384 | | ++++ | nd | +++ | nd | nd | +++ |
| 385 | D | +++++ | nd | +++ | nd | nd | nd |
| 386 | D | +++++ | nd | ++++ | nd | nd | nd |
| 386 | D | ++++ | nd | +++ | nd | nd | nd |
| 388 | Z | +++++ | nd | ++++ | nd | nd | +++ |
| 53 | Z | ++++ | nd | ++ | nd | nd | nd |
| 390 | Z | +++++ | nd | ++++ | nd | nd | ++++ |
| 391 | Z | +++++ | ++++++ | +++++ | ++++++ | nd | ++++ |
| 392 | D | +++++ | nd | +++ | nd | nd | +++ |
| 393 | D | ++++++ | nd | +++++ | nd | nd | +++ |
| 394 | Z | +++++ | ++++++ | +++++ | ++++++ | nd | +++ |
| 395 | Z | +++++ | ++++++ | ++++ | +++++ | nd | +++ |
| 396 | Z | +++++ | nd | ++++ | nd | nd | +++ |
| 397 | Z | +++++ | nd | ++++ | nd | nd | +++ |
| 398 | E | ++++++ | ++++++ | +++++ | ++++++ | nd | +++ |
| 399 | E | ++++++ | nd | ++++ | nd | nd | +++ |
| 400 | F | +++++ | nd | +++++ | nd | nd | +++ |
| 401 | F | +++++ | nd | ++++ | nd | nd | +++ |
| 402 | E | +++++ | nd | +++++ | nd | nd | +++ |
| 432 | E | +++++ | nd | +++++ | nd | nd | +++ |
| 404 | E | ++++++ | ++++++ | +++++ | ++++++ | nd | ++ |
| 405 | E | ++++++ | +++++++ | ++++++ | +++++++ | nd | +++ |
| 406 | E | ++++++ | +++++++ | +++++ | ++++++ | nd | +++ |
| 407 | F | +++++ | nd | +++++ | nd | nd | +++ |
| 408 | F | +++++ | ++++++ | +++++ | +++++ | nd | +++ |
| 409 | G | +++++ | nd | ++++ | nd | nd | +++ |
| 410 | G | ++++++ | nd | ++++++ | nd | nd | +++ |
| 411 | G | +++++ | nd | +++++ | nd | nd | +++ |
| 412 | G | ++++ | nd | +++ | nd | nd | +++ |
| 413 | G | +++++ | nd | ++++ | nd | nd | +++ |
| 414 | G | ++++++ | nd | +++++ | nd | nd | +++ |
| 415 | G | +++++ | nd | ++++ | nd | nd | +++ |
| 416 | G | ++++++ | nd | +++++ | nd | nd | +++ |
| 417 | G | ++++++ | nd | +++++ | nd | nd | +++ |
| 418 | G | ++++++ | nd | +++++ | nd | nd | +++ |
| 419 | G | ++++++ | nd | +++++ | nd | nd | +++ |
| 420 | G | ++++++ | nd | +++++ | nd | nd | +++ |
| 421 | G | ++++++ | nd | ++++ | nd | nd | +++ |
| 422 | G | ++++++ | nd | +++++ | nd | nd | +++ |
| 423 | G | +++++ | nd | ++++ | nd | nd | +++ |
| 424 | G | +++++ | nd | ++++ | nd | nd | +++ |
| 425 | G | ++++++ | nd | +++++ | nd | nd | +++ |
| 426 | Z | ++++++ | nd | ++++++ | nd | nd | +++ |
| 427 | G | ++++++ | nd | +++++ | nd | nd | +++ |
| 428 | G | ++++++ | nd | ++++++ | nd | nd | +++ |
| 429 | G | ++++++ | nd | +++++ | nd | nd | +++ |
| 430 | G | ++++++ | nd | +++++ | nd | nd | +++ |
| 431 | G | ++++++ | nd | +++++ | nd | nd | +++ |
| 432 | G | ++++++ | nd | +++++ | nd | nd | +++ |
| 433 | G | +++++ | nd | +++++ | nd | nd | +++ |
| 434 | Z | ++++++ | nd | +++++ | nd | nd | +++ |
| 435 | Z | ++++++ | nd | +++++ | nd | nd | +++ |
| 436 | G | ++++++ | nd | +++++ | nd | nd | +++ |
| 437 | Z | ++++++ | nd | +++++ | nd | nd | +++ |
| 438 | G | ++++++ | nd | +++++ | nd | nd | +++ |
| 439 | G | ++++++ | nd | +++++ | nd | nd | +++ |
| 440 | Z | ++++++ | nd | +++++ | nd | nd | +++ |

+ indicates IC₅₀ values in the range including 25 (μM) to 50 (μM)
++ indicates IC₅₀ values in the range including 10 (μM) to less than 25 (μM)
+++ indicates IC₅₀ values in the range including 5 (μM) to less than 10 (μM)
++++ indicates IC₅₀ values in the range including 1 (μM) to less than 5 μM)
+++++ indicates IC₅₀ values in the range including 0.1 (μM) to less than 1 μM)
++++++indicates IC₅₀ values in the range including 0.01 (μM) to less than 0.1 μM)
+++++++ indicates IC₅₀ values < 0.01 (μM)
nd indicates not determined

1. A compound of Formula (I), or a stereoisomer thereof or a pharmaceutically acceptable salt thereof, 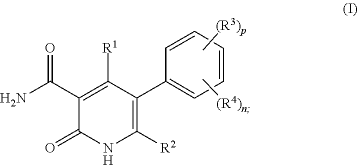
wherein:
R¹ is H or halo;
R² is —C₁-C₈haloalkyl;
R³ is L₁R⁵ or L₂R;
R⁴ is halo, CN, C₁-C₈alkyl, C₁-C₈alkoxy or C₃-C₈cycloalkyl;
L₁ is a bond, —CH₂—, —(CH₂)ₘ—, —OCH₂—, —O—, —CH₂O—, —O(CH₂)ₘ—, —CH₂OCH₂—, —CH(R⁷)—, —OCH(R⁷)—, —CH(R¹⁰)—, —OCH(R¹⁰)—, —CF₂—, —CF₂CH₂—, —OCF₂—, —OCH₂C(═O)—, —OCH₂C(═O)NH—, —OCH₂CH(OH)CH₂—, —CH₂OCH₂C(R⁷)₂—, —C(R⁷)—, —OC(R⁷)₂—, —OCH(R¹⁰)CH₂—, —NH— or —NH(CH₂)ₘ—;
L₂ is a bond, —CH₂—, —(CH₂)ₘ—, —OCH₂—, —CH₂O—, —O(CH₂)ₘ—, —CH₂OCH₂—, —CH(R⁷)—, —OCH(R⁷)—, —CH(R¹⁰)—, —OCH(R¹⁰)—, —C(R⁷)₂—, —OC(R⁷)₂—, —CF₂—, —CF₂CH₂—, —OCF₂—, —OCH₂C(═O)NH—, —OCH₂CH(OH)CH₂—, —CH₂OCH₂C(R⁷)₂—, —NH(CH₂)ₘ—, —OCH(R¹⁰)CH₂— or —OCH₂C(═O)—;
L₃ is a bond, —CH₂—, —(CH₂)ₘ—, —OCH₂—, —O—, —NH—, —NH(CH₂)ₘ—, —OCH₂C(═O)—, —OCH₂C(═O)NH—, —OCH₂CH(OH)CH₂—, —CH₂OCH₂—, —CH₂O—, —O(CH₂)ₘ—, —CH(R⁷)—, —OCH(R⁷)—, —CH(R¹⁰)—, —OCH(R¹⁰)—, —CH₂OCH₂C(R⁷)₂—, —CF₂—, —CF₂CH₂—, —OCF₂—, —C(R⁷)₂—, —OC(R⁷)₂—, or —OCH(R¹⁰)CH₂—;
L₄ is a bond, —CH₂—, —(CH₂)ₘ—, —C(R⁷)₂—, —OC(R⁷)₂, —CF₂—, —OCH₂—, —O(CH₂)ₘ—, —CH₂O—, —CH₂OCH₂—, —CH₂OCH₂C(R⁷)₂—, —NH(CH₂)ₘ—, —OCH₂C(═O)—, —OCH₂C(═O)NH—, —OCH₂CH(OH)CH₂—, —CF₂CH₂—, —OCF₂—, —CH(R¹⁰)—, —OCH(R¹⁰)—, —OCH(R⁷)—, —OC(R⁷)₂—, —CH(R⁷)—, or —OCH(R¹⁰)CH₂;
R⁵ is selected from the group consisting of
i) phenyl substituted with 0-3 groups independently selected from R⁹;
ii) C₃-C₈cycloalkyl substituted with 0-3 groups independently selected from R⁹;
iii) a 4-8 membered monocyclic heterocycloalkyl group having 1 to 3 ring members independently selected from N, NR⁷, O, S, C═O or S(═O)₂, substituted with 0-3 groups independently selected from R⁹;
iv) a 9-12 membered heterocyclyl group having 1 to 4 ring members independently selected from N, NR⁷, O, or S, substituted with 0-3 groups independently selected from R⁹;
v) a 7-8 membered bridged-bicyclic heterocycloalkyl group having 1 to 2 ring members independently selected from N, NR⁷, O, S, C═O or S(═O)₂, substituted with 0-3 groups independently selected from R⁹;
vi) a 6-8 membered fused bicyclic heterocycloalkyl group having 1 to 2 ring members independently selected from N, NR⁷, O, S, C═O or S(═O)₂, substituted with 0-3 groups independently selected from R⁹;
vii) a 5-6 membered heteroaryl having 1 to 3 ring members independently selected from N, NR⁷, O or S, substituted with 0-3 groups independently selected from R⁹
and
viii) a 9-10 membered heteroaryl having 1 to 4 ring members independently selected from N, NR⁷, N⁺O⁻, O, and S, substituted with 0-3 groups independently selected from R⁹;
R⁶ is —CN, —OH, —NR⁷R⁸, —NR⁷C(═O)R⁸, —NR⁷S(═O)₂R⁸, —C(═O)NR⁷R⁸, —S(═O)₂R⁸, halo, C₁-C₈haloalkoxy, C₁-C₈-alkoxy, —C₁-C₈alkyl or —C₂-C₈alkylene;
each R⁷ is independently selected from H and —C₁-C₈alkyl;
R⁸ is H or —C₁-C₈alkyl;
each R⁹ is independently selected from —C₁-C₈alkyl, —C₁-C₈-alkoxy, C₁-C₈haloalkyl, spiro attached C₃-C₈cycloalkyl, R¹⁰, —OH, CN, halo, —C(═O)R⁷, —NR⁷R⁸, —C(═O)NR⁷R⁸, —NR⁷C(═O)R⁸, —NR⁷C(═O)OR⁸, —C(═O)OR⁸, —NR⁷S(═O)₂R⁸, —S(═O)₂R⁸, —S(═O)₂R¹⁰, NO₂, CN, L₃R¹¹, L₄R¹² and a spiro attached 4-8 membered monocyclic heterocycloalkyl group having 1 to 2 ring members independently selected from N, NR⁷, O, S
R¹⁰ is a C₃-C₈cycloalkyl, a 4-8 membered monocyclic heterocycloalkyl group having 1 to 2 ring members independently selected from N, NR⁷, O or S, or a 5-6 membered heteroaryl having 1 to 2 ring members independently selected from N, NR⁷, O, or S;
each R¹¹ is independently selected from the group consisting of
i) phenyl substituted with 0-3 groups independently selected from R¹³;
ii) C₃-C₈cycloalkyl substituted with 0-3 groups independently selected from R¹³;
iii) a 4-8 membered monocyclic heterocycloalkyl group having 1 to 3 ring members independently selected from N, NR⁷, O, S, C═O or S(═O)₂ substituted with 0-3 groups independently selected from R¹³;
iv) a 9-12 membered heterocyclyl having 1 to 4 ring members independently selected from N, NR⁷, O, or S substituted with 0-3 groups independently selected from R¹³;
v) a 7-8 membered bridged-bicyclic heterocycloalkyl group having 1 to 2 ring members independently selected from N, NR⁷, O, S, C═O or S(═O)₂, substituted with 0-3 groups independently selected from R¹³
vi) a 6-8 membered fused bicyclic heterocycloalkyl group having 1 to 2 ring members independently selected from N, NR⁷, O, S, C═O or S(═O)₂, substituted with 0-3 groups independently selected from R¹³;
vii) a 5-6 membered heteroaryl having 1 to 3 ring members independently selected from N, NR⁷, O or S substituted with 0-3 groups independently selected from R¹³
and
viii) a 9-10 membered heteroaryl having 1 to 4 ring members independently selected from N, NR⁷, N⁺O⁻, O, or S substituted with 0-3 groups independently selected from R¹³;
each R¹² is independently selected from —CN, —CD₃, —OCD₃, —OH, —NR⁷R⁸, —NR⁷C(═O)R⁸, —NR⁷S(═O)₂R⁸, —C(═O)NR⁷R⁸, —S(═O)₂R⁸, —S(═O)₂R¹⁰, halo, C₁-C₈haloalkoxy, C₁-C₈-alkoxy, C₁-C₈haloalkyl or —C₁-C₈alkyl;
each R¹³ is independently selected from —C₁-C₈alkyl, spiro attached C₃-C₈cycloalkyl, —C₁-C₈-alkoxy, —OH, halo, —C(═O)R⁷, —NR⁷R⁸, —C(═O)NR⁷R⁸, —NR⁷C(═O)R⁸, —NR⁷S(═O)₂R⁸, —S(═O)₂R⁸, NO₂, CN or C₁-C₈haloalkyl;
each m is independently selected from 1, 2, 3 or 4;
each n is independently selected from 0, 1, 2 or 3; and
each p is independently selected from 0, 1, 2 or 3.

2. The compound of claim 1 having the Formula (II), or a stereoisomer thereof or a pharmaceutically acceptable salt thereof,

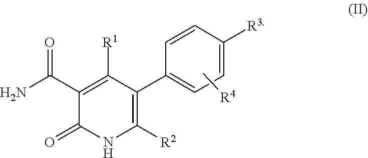

3. The compound of claim 1 or claim 2, wherein
R¹ is H;
R² is —C₁-C₈haloalkyl;
R³ is L₁R⁵ or L₂R;
R⁴ is halo, CN, C₁-C₈alkyl, C₁-C₈alkoxy or C₃-C₈cycloalkyl;
L₁ is a —CH₂—, —OCH₂—, —O—, —CH₂O—, —O(CH₂)ₘ—, —CH(R⁷)—, —OCH(R⁷)—, —CH(R¹⁰)—, —OCH(R¹⁰)—, —CF₂CH₂—, —OCF₂—, —OCH₂C(═O)—, —OCH₂C(═O)NH—, or —OCH₂CH(OH)CH₂—;
L₂ is a bond, —CH₂—, —OCH₂—, —O(CH₂)ₘ—, —CH(R⁷)—, —OCH(R¹⁰)—, —OCH(R¹⁰)CH₂— or —OCH₂C(═O)—;
L₃ is a bond, —CH₂—, —OCH₂—, —O—, —NH—, —NH(CH₂)ₘ—, —CH₂OCH₂— or —CH₂O—;
L₄ is CH₂—, —(CH₂)ₘ—, —C(R⁷)₂—, —CF₂—, —OCH₂—, —O(CH₂)ₘ—, —CH₂O—, —CH₂OCH₂—, —CH₂OCH₂C(R⁷)₂— or —NH(CH₂)ₘ—;
R⁵ is selected from the group consisting of
i) phenyl substituted with 0-3 groups independently selected from R⁹;
ii) C₃-C₈cycloalkyl substituted with 0-3 groups independently selected from R⁹;
iii) a 4-8 membered monocyclic heterocycloalkyl group having 1 to 3 ring members independently selected from N, NR⁷, O, S, C═O or S(═O)₂, substituted with 0-3 groups independently selected from R⁹;
iv) a 9-12 membered heterocyclyl group having 1 to 4 ring members independently selected from N, NR⁷, O, or S, substituted with 0-3 groups independently selected from R⁹;
v) a 7-8 membered bridged-bicyclic heterocycloalkyl group having 1 to 2 ring members independently selected from N, NR⁷, O or S, substituted with 0-3 groups independently selected from R⁹;
vi) a 6-8 membered fused bicyclic heterocycloalkyl group having 1 to 2 ring members independently selected from N, NR⁷, O and S, substituted with 0-3 groups independently selected from R⁹;
vii) a 5-8 membered heteroaryl having 1 to 3 ring members independently selected from N, NR⁷, O or S, substituted with 0-3 groups independently selected from R⁹
and
viii) a 9-10 membered heteroaryl having 1 to 4 ring members independently selected from N, NR⁷, N⁺O⁻, O, or S, substituted with 0-3 groups independently selected from R⁹;
R⁶ is —CN, —OH, —NR⁷R⁸, —NR⁷C(═O)R⁸, —NR⁷S(═O)₂R⁸, —C(═O)NR⁷R⁸, —S(═O)₂R⁸, halo, C₁-C₈haloalkoxy, C₁-C₈-alkoxy, —C₁-C₈alkyl or —C₂-C₈alkylene;
each R⁷ is independently selected from H or —C₁-C₈alkyl;
R⁸ is H or —C₁-C₈alkyl;
each R⁹ is independently selected from —C₁-C₈alkyl, —C₁-C₈-alkoxy, C₁-C₈haloalkyl, spiro attached C₃-C₈cycloalkyl, R¹⁰, —OH, CN, halo, —C(═O)R⁷, —NR⁷R⁸, —C(═O)NR⁷R⁸, —NR⁷C(═O)R⁸, —C(═O)OR⁸, —NR⁷S(═O)₂R⁸, —S(═O)₂R⁸, —S(═O)₂R¹⁰, NO₂, CN, L₃R¹¹, L₄R¹² and a spiro attached 4-8 membered monocyclic heterocycloalkyl group having 1 to 2 ring members independently selected from N, NR⁷, O, or S;
R¹⁰ is a C₃-C₈cycloalkyl, a 4-8 membered monocyclic heterocycloalkyl group having 1 to 2 ring members independently selected from N, NR⁷, O or S, or a 5-8 membered heteroaryl having 1 to 2 ring members independently selected from N, NR⁷, O, or S;
each R¹¹ is independently selected from the group consisting of
i) phenyl substituted with 0-3 groups independently selected from R¹³;
ii) C₃-C₈cycloalkyl substituted with 0-3 groups independently selected from R¹³;
iii) a 4-8 membered monocyclic heterocycloalkyl group having 1 to 3 ring members independently selected from N, NR⁷, O or S, substituted with 0-3 groups independently selected from R¹³;
and
iv) a 5-8 membered heteroaryl having 1 to 3 ring members independently selected from N, NR⁷, O or S substituted with 0-3 groups independently selected from R¹³;
each R¹² is independently selected from —CN, —CD₃, —OCD₃, —OH, —NR⁷R⁸, —NR⁷C(═O)R⁸, —NR⁷S(═O)₂R⁸, —C(═O)NR⁷R⁸, —S(═O)₂R⁸, —S(═O)₂R¹⁰, halo, C₁-C₈haloalkoxy, C₁-C₈haloalkyl, —C₁-C₈alkyl or C₁-C₈-alkoxy;
each R¹³ is independently selected from —C₁-C₈alkyl, —C₁-C₈-alkoxy, —OH or halo;
each m is independently selected from 1, 2 or 3,
and
each p is independently selected from 0 or 1.

4. The compound of any one of claims 1 to 3 having the Formula (III), or a stereoisomer thereof or a pharmaceutically acceptable salt thereof,

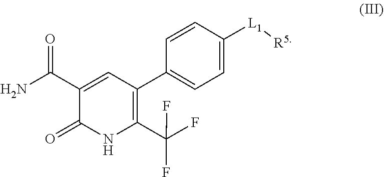

5. The compound of any one of claims 1 to 4 having the Formula (IV), or a stereoisomer thereof or a pharmaceutically acceptable salt thereof,

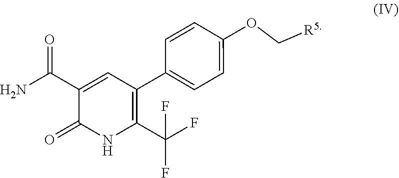

6. The compound of any one of claims 1 to 5, or a stereoisomer thereof or a pharmaceutically acceptable salt thereof, wherein
R⁵ is selected from the group consisting of
i) phenyl substituted with 0-3 groups independently selected from R⁹;
ii) cyclopropyl, cyclobutyl or cyclohexyl, each of which is substituted with 0-3 groups independently selected from R⁹;
iii) morpholinyl, pyrrolidinyl, thiomorpholinyl, oxetanyl, oxazepanyl, azetidinyl, pyrrolidinyl, piperidinyl, oxomorpholinyl, dioxidothiomorpholinyl, tetrahydropyranyl, tetrahydrofuranyl, dioxanyl, dioxepanyl, oxopyrrolidinyl, or oxooxazolidinyl, each of which is substituted with 0-3 groups independently selected from R⁹;
iv) benzo[d][1,3]dioxolyl, benzo[b][1,4]dioxepinyl, tetrahydrobenzo[d]isoxazolyl, dihydrobenzofuranyl, tetrahydropyrano[3,4-c]pyrazolyl, chromanyl or 6,7-dihydro-4H-[1,2,3]triazolo[5,1-c][1,4]oxazinyl each of which is substituted with 0-3 groups independently selected from R⁹;
v) 8-oxa-3-azabicyclo[3.2.1]octanyl, oxabicyclo[2.2.1]heptanyl, or 3,8-dioxabicyclo[3.2.1]octanyl, each of which is substituted with 0-3 groups independently selected from R⁹;
vi) 3-azabicyclo[3.1.0]hexanyl or 2,5-dioxabicyclo[4.1.0]heptanyl, each of which is substituted with 0-3 groups independently selected from R⁹;
vii) oxazolyl, isoxazolyl, pyrazolyl, thiophenyl, thiazolyl, or thiadiazolyl, imidazolyl, pyridinyl, pyrimidinyl, pyridazinyl or pyrazinyl, each of which is substituted with 0-3 groups independently selected from R⁹,
and
viii) benzo[c][1,2,5]oxadiazolyl, indazolyl, tetrazolo[1,5-a]pyridinyl, imidazo[1,2-a]pyridinyl, imidazo[4,5-b]pyridinyl, triazolo[4,3-a]pyrimidinyl, quinoxalinyl, quinolinyl, or isoquinolinyl, each of which is substituted with 0-3 groups independently selected from R⁹.

7. The compound of any one of claims 1 to 6 having the Formula (V), or a stereoisomer thereof or a pharmaceutically acceptable salt thereof,

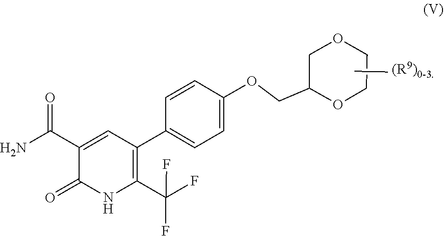

8. The compound of any one of claims 1 to 7 having the Formula (V-a), Formula (V-b) or Formula (V-c), or a stereoisomer thereof or a pharmaceutically acceptable salt thereof,

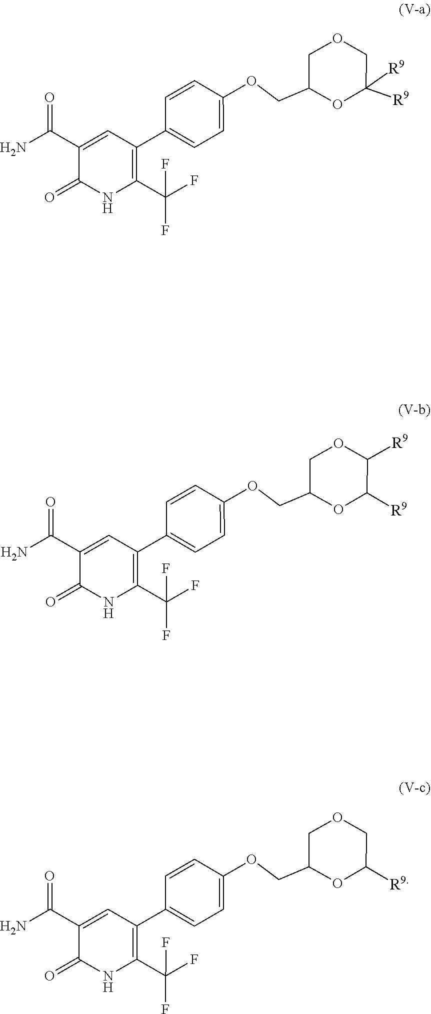

9. The compound of any one of claims 1 to 8 having the Formula (V-a1) or Formula (V-a2), or a pharmaceutically acceptable salt thereof,

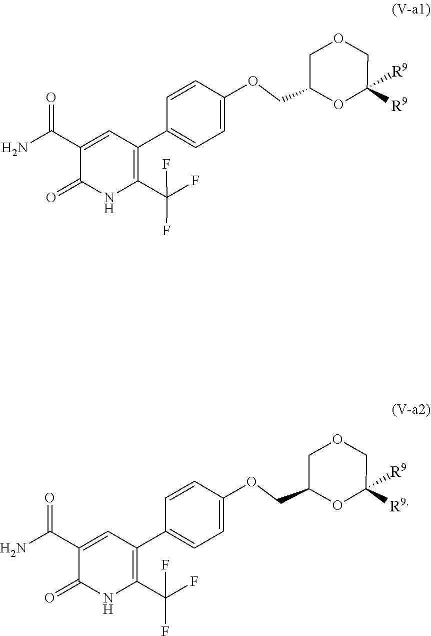

10. The compound of any one of claims 1 to 9, wherein each R⁹ is independently selected from methyl, ethyl, isopropyl, neopentyl, methoxy, ethoxy, propoxy, isopropoxy, —CH₂F, —CHF₂, —CF₃, R¹⁰, —OH, CN, F, C₁, Br, —C(═O)CH₃, —NH₂, —NHCH₃, —NHCH₂CH₃, —NHCH₂CH₂CH₃, —NH(CH₂)₆CH₃, —N(CH₃)₂, —C(═O)NH₂, —C(═O)N(CH₃)₂, —C(═O)NHCH₃, —NHC(═O)CH₃, —NHC(═O)OC(CH₃)₃, —C(═O)OC(CH₃)₃, —NHS(═O)₂CH₃, —S(═O)₂CH₃, —S(═O)₂CH(CH₃)₂, —S(═O)₂CH₂CH(CH₃)₂, —S(═O)₂-cyclopropyl, NO₂, CN, L₃R¹¹, L₄R¹², cyclopropyl, cyclopentyl, spiro attached cyclopropyl, spiro attached oxetanyl, spiro attached dioxepanyl or spiro attached tetrahydrofuranyl.

11. The compound of any one of claims 1 to 9, wherein each R⁹ is independently selected from —C₁-C₈alkyl, —C₁-C₈alkoxy, halo, C₁-C₈haloalkyl, spiro attached C₃-C₈cycloalkyl, R¹⁰, L₃R¹¹, L₄R¹² or a spiro attached 4-8 membered monocyclic heterocycloalkyl group having 1 to 2 ring members independently selected from N, NR⁷, O, or S.

12. The compound of any one of claims 1 to 11, wherein each R⁹ is independently selected from methyl, F, methoxy, —CH₂F, —CHF₂, L₃R¹¹, L₄R¹², —C(═O)N(CH₃)₂, cyclopropyl, cyclopentyl, spiro attached cyclopropyl, spiro attached oxetanyl, spiro attached dioxepanyl or spiro attached tetrahydrofuranyl.

13. The compound of any one of claims 1 to 12, wherein each R⁹ is independently selected from methyl, L₃R¹¹ or L₄RU.

14. The compound of any one of claims 1 to 13, wherein each R⁹ is independently selected from methyl or L₃R¹¹, and wherein
L₃ is a bond, —CH₂—, —OCH₂—, —O—, —NH—, —NH(CH₂)ₘ—, —CH₂OCH₂—, —CH₂O—, —O(CH₂)ₘ—, —CH(R⁷)—, —OCH(R⁷)—, —CH(R¹⁰)—, —CF₂CH₂—, —C(R⁷)₂—, or —OC(R₇)₂—;
R¹¹ is phenyl, tetrahydrofuranyl, cyclobutyl, cyclopropyl, cyclopentyl, oxetanyl, azetidinyl, pyrrolyl or pyrazolyl, each of which is substituted with 0 to 2 R¹³ groups, each of which is substituted with 0 to 2 R¹³ groups,
and
each R¹³ is independently selected from F, OH, methoxy or methyl.

15. The compound of any one of claims 1 to 14, wherein each R⁹ is independently selected from methyl or L₃R¹¹, and wherein
L₃ is bond, —CH₂, —OCH₂—, —O—, —NH—, —NHCH₂—, —CH₂OCH₂— or —CH₂O—;
R¹¹ is phenyl, tetrahydrofuranyl, cyclobutyl, cyclopropyl, cyclopentyl, oxetanyl, azetidinyl, pyrrolyl or pyrazolyl, each of which is substituted with 0 to 2 R¹³ groups,
and
each R¹³ is independently selected from F, OH, methoxy or methyl.

16. The compound of any one of claims 1 to 13, wherein each R⁹ is independently selected from methyl or L₄R¹², and wherein
L₄ is —OCH2CH₂—, —NHCH₂CH₂—, —CH₂—, —CH₂O—, —CH₂CH₂—, —C(CH₃)₂—, —C(CH₂CH₃)₂—, —CF₂—, —CH₂OCH₂—, —CH₂OCH₂CH₂— or —CH₂OCH₂C(CH₃)₂—;
and
R¹² is OH, CN, CD₃, OCD₃, F, C₁, —OCH₂F, —OCHF₂, OCF₃, OCH₂CH₂F, OCH₂CHF₂, OCH₂CHF₃, —CH₂F, CHF₂, CF₃, —CH₂CH₂F, —CH₂CHF₂, —CH₂CHF₃, methoxy, ethoxy, isopropoxy, CN, methyl, ethyl, isopropyl, —S(═O)₂CH₃, —S(═O)₂-cyclopropyl or —N(CH₃)₂.

17. The compound of any one of claims 1 to 13 having the Formula (V-b1), Formula (V-b2), Formula (V-b3) or Formula (V-b4), or a pharmaceutically acceptable salt thereof,

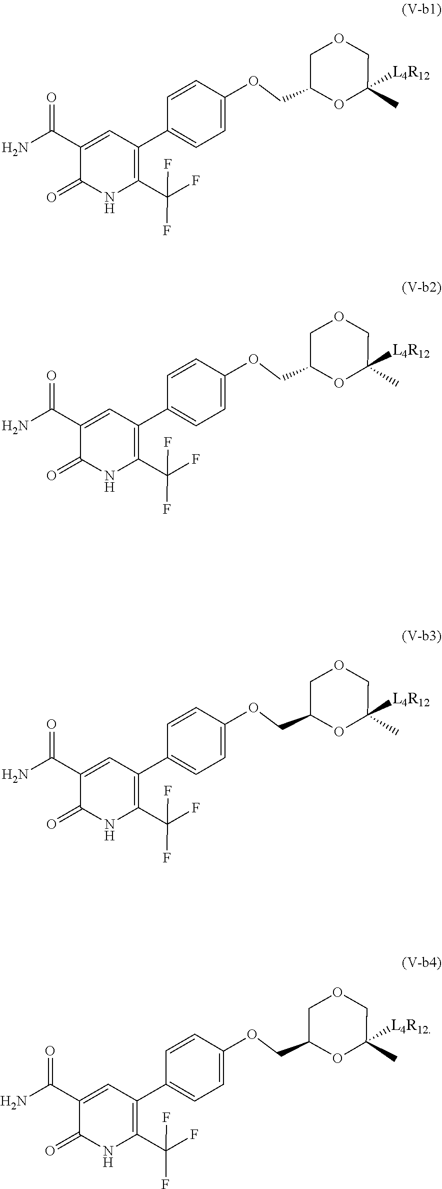

18. The compound of any one of claims 1 to 13 having the Formula (V-c1), Formula (V-c2), Formula (V-c3) or Formula (V-c4), or a pharmaceutically acceptable salt thereof,

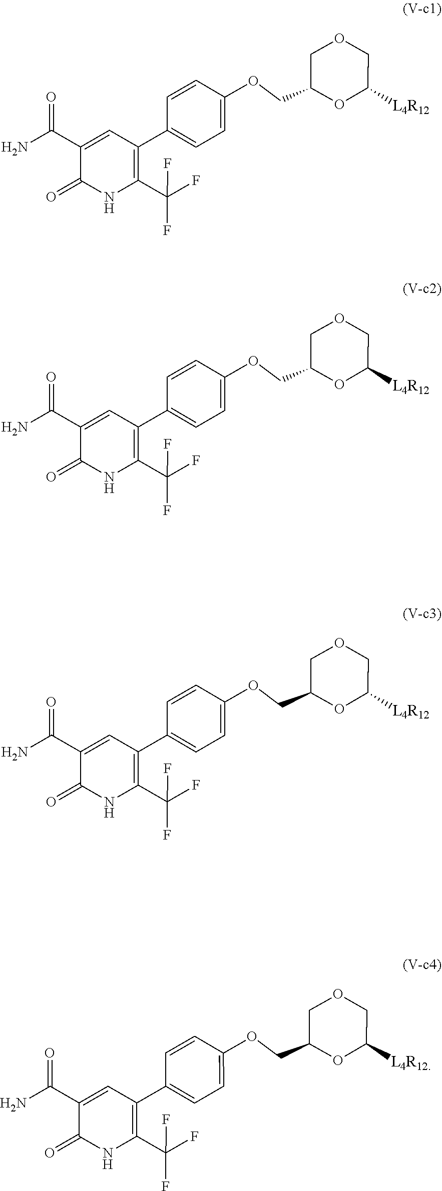

19. The compound of any one of claims 1 to 18, wherein
L₄ is —OCH₂CH₂—, —NHCH₂CH₂—, —CH₂—, —CH₂O—, —CH₂CH₂—, —C(CH₃)₂—, —C(CH₂CH₃)₂—, —CF₂—, —CH₂OCH₂—, —CH₂OCH₂CH₂— or —CH₂OCH₂C(CH₃)₂—;
R¹² is OH, CN, CD₃, OCD₃, F, Cl, —OCH₂F, —OCHF₂, OCF₃, OCH₂CH₂F, OCH₂CHF₂, OCH₂CHF₃, —CH₂F, CHF₂, CF₃, —CH₂CH₂F, —CH₂CHF₂, —CH₂CHF₃, methoxy, ethoxy, isopropoxy, CN, methyl, ethyl, isopropyl, —S(═O)₂CH₃, —S(═O)₂-cyclopropyl or —N(CH₃)₂.

20. The compound of any one of claims 1 to 19, wherein L₄ is a bond, —CH₂—, —(CH₂)ₘ—, —C(R⁷)₂—, —CF₂—, —OCH₂—, —O(CH₂)ₘ—, —CH₂O—, —CH₂OCH₂—, or —CH₂OCH₂C(R⁷)₂—.

21. The compound of any one of claims 1 to 20, wherein L₄ is a bond, —CH₂—, —(CH₂)ₘ—, —C(CH₃)₂—, —C(CH₂CH₃)₂—, —CF₂—, —CH₂O—, —CH₂OCH₂—, —CH₂OCH₂C(CH₃)₂— or —CH₂OCH₂CH₂.

22. The compound of any one of claims 1 to 21, wherein L₄ is —CH₂—, or —CH₂O—.

23. The compound of any one of claims 1 to 22 having the Formula (V-d1), Formula (V-d2), Formula (V-d3) or Formula (V-d4) or a pharmaceutically acceptable salt thereof,

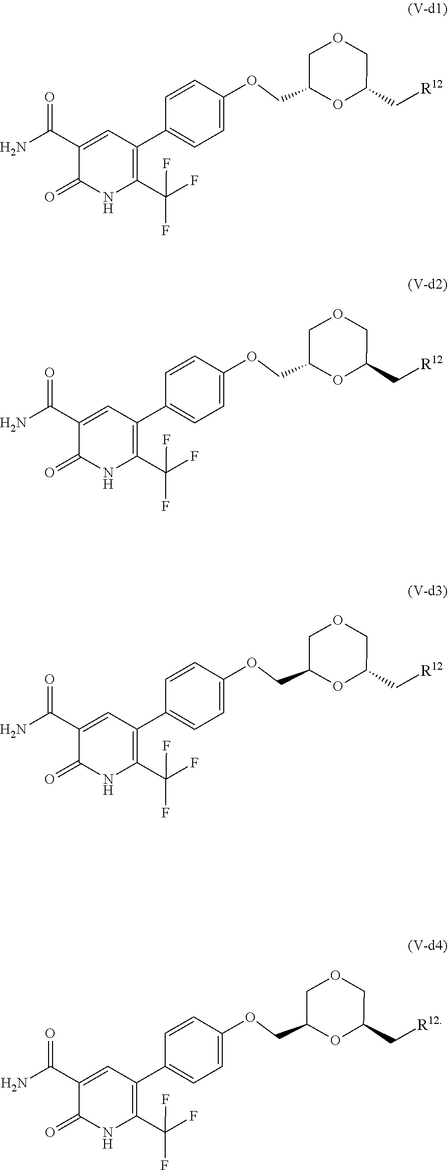

24. The compound of any one of claims 1 to 13 having the Formula (V-e1), Formula (V-e2), Formula (V-e3) or Formula (V-e4), or a pharmaceutically acceptable salt thereof,

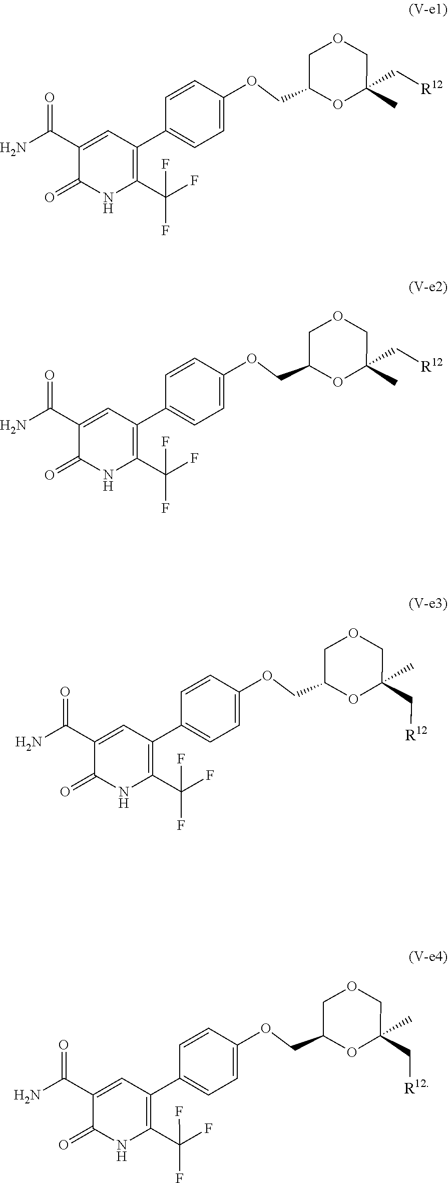

25. The compound of any one of claims 1 to 24, wherein R¹² is CN, CD₃, OCD₃, OH, N(CH₃)₂, —S(O)₂CH₃, —S(O)₂-cyclopropyl, F, Cl, —OCHF₂, —OCHF₂, OCF₃, CH₂F, CHF₂, CF₃, -methyl, methoxy, ethoxy or isopropoxy.

26. The compound of any one of claims 1 to 25, wherein R¹² is methoxy, ethoxy or isopropoxy.

27. A compound of claim 1, wherein the compound is:
5-(4-(morpholinomethyl)phenyl)-2-oxo-6-(trifluoromethyl)-1,2-dihydropyridine-3-carboxamide;
5-(4-(hydroxymethyl)phenyl)-2-oxo-6-(trifluoromethyl)-1,2-dihydropyridine-3-carboxamide;
5-(4-hydroxyphenyl)-2-oxo-6-(trifluoromethyl)-1,2-dihydropyridine-3-carboxamide;
5-(4-(benzyloxy)phenyl)-2-oxo-6-(trifluoromethyl)-1,2-dihydropyridine-3-carboxamide;
2-oxo-5-(4-(pyrrolidin-1-ylmethyl)phenyl)-6-(trifluoromethyl)-1,2-dihydropyridine-3-carboxamide;
2-oxo-5-(4-phenoxyphenyl)-6-(trifluoromethyl)-1,2-dihydropyridine-3-carboxamide;
5-(3-aminophenyl)-2-oxo-6-(trifluoromethyl)-1,2-dihydropyridine-3-carboxamide;
5-(3-cyanophenyl)-2-oxo-6-(trifluoromethyl)-1,2-dihydropyridine-3-carboxamide;
5-(3-hydroxyphenyl)-2-oxo-6-(trifluoromethyl)-1,2-dihydropyridine-3-carboxamide;
5-(3-formamidophenyl)-2-oxo-6-(trifluoromethyl)-1,2-dihydropyridine-3-carboxamide;
5-(4-(methylsulfonamido)phenyl)-2-oxo-6-(trifluoromethyl)-1,2-dihydropyridine-3-carboxamide;
5-(4-(difluoromethoxy)phenyl)-2-oxo-6-(trifluoromethyl)-1,2-dihydropyridine-3-carboxamide;
5-(4-(methoxymethoxy)phenyl)-2-oxo-6-(trifluoromethyl)-1,2-dihydropyridine-3-carboxamide;
5-(3-fluoro-4-methoxyphenyl)-2-oxo-6-(trifluoromethyl)-1,2-dihydropyridine-3-carboxamide;
5-(4-(methoxymethyl)phenyl)-2-oxo-6-(trifluoromethyl)-1,2-dihydropyridine-3-carboxamide;
2-oxo-5-phenyl-6-(trifluoromethyl)-1,2-dihydropyridine-3-carboxamide;
5-(4-isopropoxyphenyl)-2-oxo-6-(trifluoromethyl)-1,2-dihydropyridine-3-carboxamide;
5-(3-chloro-4-methoxyphenyl)-2-oxo-6-(trifluoromethyl)-1,2-dihydropyridine-3-carboxamide;
5-(4-(methylcarbamoyl)phenyl)-2-oxo-6-(trifluoromethyl)-1,2-dihydropyridine-3-carboxamide;
5-(4-(cyanomethyl)phenyl)-2-oxo-6-(trifluoromethyl)-1,2-dihydropyridine-3-carboxamide;
5-(4-(3-(dimethylamino)propoxy)phenyl)-2-oxo-6-(trifluoromethyl)-1,2-dihydropyridine-3-carboxamide;
5-(3-(cyanomethyl)phenyl)-2-oxo-6-(trifluoromethyl)-1,2-dihydropyridine-3-carboxamide;
5-(3-(morpholinomethyl)phenyl)-2-oxo-6-(trifluoromethyl)-1,2-dihydropyridine-3-carboxamide;
2-oxo-5-(m-tolyl)-6-(trifluoromethyl)-1,2-dihydropyridine-3-carboxamide;
5-(3-(hydroxymethyl)phenyl)-2-oxo-6-(trifluoromethyl)-1,2-dihydropyridine-3-carboxamide;
2-oxo-5-(p-tolyl)-6-(trifluoromethyl)-1,2-dihydropyridine-3-carboxamide;
5-(4-((3-methylmorpholino)methyl)phenyl)-2-oxo-6-(trifluoromethyl)-1,2-dihydropyridine-3-carboxamide;
2-oxo-5-(4-(thiomorpholinomethyl)phenyl)-6-(trifluoromethyl)-1,2-dihydropyridine-3-carboxamide;
5-(4-((2-methylmorpholino)methyl)phenyl)-2-oxo-6-(trifluoromethyl)-1,2-dihydropyridine-3-carboxamide;
5-(4-((7-oxa-4-azaspiro[2.5]octan-4-yl)methyl)phenyl)-2-oxo-6-(trifluoromethyl)-1,2-dihydropyridine-3-carboxamide;
5-(4-(((1R,5S)-8-oxa-3-azabicyclo[3.2.1]octan-3-yl)methyl)phenyl)-2-oxo-6-(trifluoromethyl)-1,2-dihydropyridine-3-carboxamide;
2-oxo-5-(4-((3-(pyridin-3-yl)morpholino)methyl)phenyl)-6-(trifluoromethyl)-1,2-dihydropyridine-3-carboxamide;
5-(4-(1-morpholinoethyl)phenyl)-2-oxo-6-(trifluoromethyl)-1,2-dihydropyridine-3-carboxamide;
5-(4-((1,4-oxazepan-4-yl)methyl)phenyl)-2-oxo-6-(trifluoromethyl)-1,2-dihydropyridine-3-carboxamide;
5-(4-(((2S,6R)-2,8-dimethylmorpholino)methyl)phenyl)-2-oxo-6-(trifluoromethyl)-1,2-dihydropyridine-3-carboxamide;
5-(3-fluoro-4-(morpholinomethyl)phenyl)-2-oxo-6-(trifluoromethyl)-1,2-dihydropyridine-3-carboxamide;
5-(4-(1-hydroxyethyl)phenyl)-2-oxo-6-(trifluoromethyl)-1,2-dihydropyridine-3-carboxamide;
5-(4-((3-methoxyazetidin-1-yl)methyl)phenyl)-2-oxo-6-(trifluoromethyl)-1,2-dihydropyridine-3-carboxamide;
5-(4-((2,2-dimethylmorpholino)methyl)phenyl)-2-oxo-6-(trifluoromethyl)-1,2-dihydropyridine-3-carboxamide;
(S)-5-(4-((3-methoxypyrrolidin-1-yl)methyl)phenyl)-2-oxo-6-(trifluoromethyl)-1,2-dihydropyridine-3-carboxamide;
(R)-5-(4-((3-methoxypyrrolidin-1-yl)methyl)phenyl)-2-oxo-6-(trifluoromethyl)-1,2-dihydropyridine-3-carboxamide;
5-(2-fluoro-4-(morpholinomethyl)phenyl)-2-oxo-6-(trifluoromethyl)-1,2-dihydropyridine-3-carboxamide;
5-(3-methyl-4-(morpholinomethyl)phenyl)-2-oxo-6-(trifluoromethyl)-1,2-dihydropyridine-3-carboxamide;
2-oxo-5-(4-(piperidin-1-ylmethyl)phenyl)-6-(trifluoromethyl)-1,2-dihydropyridine-3-carboxamide;
5-(4-((dimethylamino)methyl)phenyl)-2-oxo-6-(trifluoromethyl)-1,2-dihydropyridine-3-carboxamide;
2-oxo-5-(4-((3-oxomorpholino)methyl)phenyl)-6-(trifluoromethyl)-1,2-dihydropyridine-3-carboxamide;
5-(4-((1,1-dioxidothiomorpholino)methyl)phenyl)-2-oxo-6-(trifluoromethyl)-1,2-dihydropyridine-3-carboxamide;
5-(4-(morpholino(oxazol-5-yl)methyl)phenyl)-2-oxo-6-(trifluoromethyl)-1,2-dihydropyridine-3-carboxamide;
5-(4-(methylsulfonamidomethyl)phenyl)-2-oxo-6-(trifluoromethyl)-1,2-dihydropyridine-3-carboxamide;
5-(4-((4-hydroxytetrahydro-2H-pyran-4-yl)methyl)phenyl)-2-oxo-6-(trifluoromethyl)-1,2-dihydropyridine-3-carboxamide;
5-(4-((4-fluorotetrahydro-2H-pyran-4-yl)methyl)phenyl)-2-oxo-6-(trifluoromethyl)-1,2-dihydropyridine-3-carboxamide;
2-oxo-5-(4-((tetrahydro-2H-pyran-4-yl)methyl)phenyl)-6-(trifluoromethyl)-1,2-dihydropyridine-3-carboxamide;
5-(4-(2-(1,4-dioxan-2-yl)-1,1-difluoroethyl)phenyl)-2-oxo-6-(trifluoromethyl)-1,2-dihydropyridine-3-carboxamide;
5-(4-((3-nitrobenzyl)oxy)phenyl)-2-oxo-6-(trifluoromethyl)-1,2-dihydropyridine-3-carboxamide;
5-(4-((1-acetylpiperidin-4-yl)methoxy)phenyl)-2-oxo-6-(trifluoromethyl)-1,2-dihydropyridine-3-carboxamide;
2-oxo-5-(4-((tetrahydro-2H-pyran-4-yl)methoxy)phenyl)-6-(trifluoromethyl)-1,2-dihydropyridine-3-carboxamide;
5-(4-((3-aminobenzyl)oxy)phenyl)-2-oxo-6-(trifluoromethyl)-1,2-dihydropyridine-3-carboxamide;
5-(4-(cyclohexylmethoxy)phenyl)-2-oxo-6-(trifluoromethyl)-1,2-dihydropyridine-3-carboxamide;
5-(4-((4-methoxybenzyl)oxy)phenyl)-2-oxo-6-(trifluoromethyl)-1,2-dihydropyridine-3-carboxamide;
5-(4-((4-(dimethylcarbamoyl)benzyl)oxy)phenyl)-2-oxo-6-(trifluoromethyl)-1,2-dihydropyridine-3-carboxamide;
5-(4-((3-methoxybenzyl)oxy)phenyl)-2-oxo-6-(trifluoromethyl)-1,2-dihydropyridine-3-carboxamide;
5-(4-((3-(methylcarbamoyl)benzyl)oxy)phenyl)-2-oxo-6-(trifluoromethyl)-1,2-dihydropyridine-3-carboxamide;
2-oxo-5-(4-((tetrahydro-2H-pyran-4-yl)oxy)phenyl)-6-(trifluoromethyl)-1,2-dihydropyridine-3-carboxamide;
5-(4-((3-(methylsulfonamido)benzyl)oxy)phenyl)-2-oxo-6-(trifluoromethyl)-1,2-dihydropyridine-3-carboxamide;
5-(4-((3-carbamoylbenzyl)oxy)phenyl)-2-oxo-6-(trifluoromethyl)-1,2-dihydropyridine-3-carboxamide;
2-oxo-5-(4-((3-(propylamino)benzyl)oxy)phenyl)-6-(trifluoromethyl)-1,2-dihydropyridine-3-carboxamide;
5-(4-((3-(dimethylcarbamoyl)benzyl)oxy)phenyl)-2-oxo-6-(trifluoromethyl)-1,2-dihydropyridine-3-carboxamide;
5-(4-((3-acetamidobenzyl)oxy)phenyl)-2-oxo-6-(trifluoromethyl)-1,2-dihydropyridine-3-carboxamide;
5-(4-((4-(methylcarbamoyl)benzyl)oxy)phenyl)-2-oxo-6-(trifluoromethyl)-1,2-dihydropyridine-3-carboxamide;
2-oxo-5-(4-(pyridin-4-ylmethoxy)phenyl)-6-(trifluoromethyl)-1,2-dihydropyridine-3-carboxamide;
5-(4-(benzo[c][1,2,5]oxadiazol-5-ylmethoxy)phenyl)-2-oxo-6-(trifluoromethyl)-1,2-dihydropyridine-3-carboxamide;
2-oxo-5-(4-(pyridin-3-ylmethoxy)phenyl)-6-(trifluoromethyl)-1,2-dihydropyridine-3-carboxamide;
2-oxo-5-(4-(pyridin-2-ylmethoxy)phenyl)-6-(trifluoromethyl)-1,2-dihydropyridine-3-carboxamide;
5-(4-(benzo[c][1,2,5]oxadiazol-4-ylmethoxy)phenyl)-2-oxo-6-(trifluoromethyl)-1,2-dihydropyridine-3-carboxamide;
5-(4-((3-(methylsulfonyl)benzyl)oxy)phenyl)-2-oxo-6-(trifluoromethyl)-1,2-dihydropyridine-3-carboxamide;
5-(4-((2-nitrobenzyl)oxy)phenyl)-2-oxo-6-(trifluoromethyl)-1,2-dihydropyridine-3-carboxamide;
5-(4-((4-cyanobenzyl)oxy)phenyl)-2-oxo-6-(trifluoromethyl)-1,2-dihydropyridine-3-carboxamide;
5-(4-((3-cyanobenzyl)oxy)phenyl)-2-oxo-6-(trifluoromethyl)-1,2-dihydropyridine-3-carboxamide;
2-oxo-6-(trifluoromethyl)-5-(4-((3-(trifluoromethylbenzyl)oxy)phenyl)-1,2-dihydropyridine-3-carboxamide;
(R or S)-2-oxo-5-(4-(1-(pyridin-3-yl)ethoxy)phenyl)-6-(trifluoromethyl)-1,2-dihydropyridine-3-carboxamide;
2-oxo-5-(4-(pyrimidin-5-ylmethoxy)phenyl)-6-(trifluoromethyl)-1,2-dihydropyridine-3-carboxamide;
5-(4-((1-methylpiperidin-4-yl)methoxy)phenyl)-2-oxo-6-(trifluoromethyl)-1,2-dihydropyridine-3-carboxamide;
2-oxo-5-(4-(pyridazin-4-ylmethoxy)phenyl)-6-(trifluoromethyl)-1,2-dihydropyridine-3-carboxamide;
5-(4-((1H-indazol-6-yl)methoxy)phenyl)-2-oxo-6-(trifluoromethyl)-1,2-dihydropyridine-3-carboxamide;
5-(4-((1H-indol-6-yl)methoxy)phenyl)-2-oxo-6-(trifluoromethyl)-1,2-dihydropyridine-3-carboxamide;
2-oxo-6-(trifluoromethyl)-5-(4-((4-(trifluoromethylbenzyl)oxy)phenyl)-1,2-dihydropyridine-3-carboxamide;
2-oxo-5-(4-((5-oxopyrrolidin-3-yl)methoxy)phenyl)-6-(trifluoromethyl)-1,2-dihydropyridine-3-carboxamide;
5-(4-((2-cyanobenzyl)oxy)phenyl)-2-oxo-6-(trifluoromethyl)-1,2-dihydropyridine-3-carboxamide;
2-oxo-5-(4-(tetrazolo[1,5-a]pyridin-7-ylmethoxy)phenyl)-6-(trifluoromethyl)-1,2-dihydropyridine-3-carboxamide;
5-(4-(imidazo[1,2-a]pyridin-7-ylmethoxy)phenyl)-2-oxo-6-(trifluoromethyl)-1,2-dihydropyridine-3-carboxamide;
5-(4-((1-methylpiperidin-3-yl)methoxy)phenyl)-2-oxo-6-(trifluoromethyl)-1,2-dihydropyridine-3-carboxamide;
5-(4-((4-fluoro-3-nitrobenzyl)oxy)phenyl)-2-oxo-6-(trifluoromethyl)-1,2-dihydropyridine-3-carboxamide;
8-((4-(5-carbamoyl-6-oxo-2-(trifluoromethyl)-1,8-dihydropyridin-3-yl)phenoxy)methyl)benzo[c][1,2,5]oxadiazole 1-oxide;
(R or S)-2-oxo-5-(4-(1-(pyrimidin-5-yl)ethoxy)phenyl)-6-(trifluoromethyl)-1,2-dihydropyridine-3-carboxamide;
(R or S)-5-(4-(1-(3-cyanophenyl)ethoxy)phenyl)-2-oxo-6-(trifluoromethyl)-1,2-dihydropyridine-3-carboxamide;
5-(4-((5-cyanopyridin-3-yl)methoxy)phenyl)-2-oxo-6-(trifluoromethyl)-1,2-dihydropyridine-3-carboxamide;
5-(4-(1-(benzo[c][1,2,5]oxadiazol-5-yl)ethoxy)phenyl)-2-oxo-6-(trifluoromethyl)-1,2-dihydropyridine-3-carboxamide;
2-oxo-5-(4-((tetrahydro-2H-pyran-3-yl)methoxy)phenyl)-6-(trifluoromethyl)-1,2-dihydropyridine-3-carboxamide;
5-(4-([1,2,4]triazolo[4,3-a]pyrimidin-6-ylmethoxy)phenyl)-2-oxo-6-(trifluoromethyl)-1,2-dihydropyridine-3-carboxamide;
5-(4-((5-chloro-2-fluoropyridin-3-yl)methoxy)phenyl)-2-oxo-6-(trifluoromethyl)-1,2-dihydropyridine-3-carboxamide;
5-(4-((5-chloropyridin-2-yl)methoxy)phenyl)-2-oxo-6-(trifluoromethyl)-1,2-dihydropyridine-3-carboxamide;
5-(4-((2-methoxypyridin-4-yl)methoxy)phenyl)-2-oxo-6-(trifluoromethyl)-1,2-dihydropyridine-3-carboxamide;
5-(4-((3,5-dimethylisoxazol-4-yl)methoxy)phenyl)-2-oxo-6-(trifluoromethyl)-1,2-dihydropyridine-3-carboxamide;
5-(4-((2-chloro-5-nitrobenzyl)oxy)phenyl)-2-oxo-6-(trifluoromethyl)-1,2-dihydropyridine-3-carboxamide;
5-(4-((1-methyl-3-(trifluoromethyl)-1H-pyrazol-4-yl)methoxy)phenyl)-2-oxo-6-(trifluoromethyl)-1,2-dihydropyridine-3-carboxamide;
2-oxo-5-(4-(thiophen-3-ylmethoxy)phenyl)-6-(trifluoromethyl)-1,2-dihydropyridine-3-carboxamide;
2-oxo-6-(trifluoromethyl)-5-(4-((8-(trifluoromethyl)pyridin-3-yl)methoxy)phenyl)-1,2-dihydropyridine-3-carboxamide;
5-(4-((3,4-difluorobenzyl)oxy)phenyl)-2-oxo-6-(trifluoromethyl)-1,2-dihydropyridine-3-carboxamide;
5-(4-((4-methoxy-3,5-dimethylpyridin-2-yl)methoxy)phenyl)-2-oxo-6-(trifluoromethyl)-1,2-dihydropyridine-3-carboxamide;
5-(4-((3-fluoropyridin-2-yl)methoxy)phenyl)-2-oxo-6-(trifluoromethyl)-1,2-dihydropyridine-3-carboxamide;
2-oxo-5-(4-(thiophen-2-ylmethoxy)phenyl)-6-(trifluoromethyl)-1,2-dihydropyridine-3-carboxamide;
5-(4-((2,5-difluorobenzyl)oxy)phenyl)-2-oxo-6-(trifluoromethyl)-1,2-dihydropyridine-3-carboxamide;
5-(4-((4-chloropyridin-2-yl)methoxy)phenyl)-2-oxo-6-(trifluoromethyl)-1,2-dihydropyridine-3-carboxamide;
5-(4-((2,4-dimethylthiazol-5-yl)methoxy)phenyl)-2-oxo-6-(trifluoromethyl)-1,2-dihydropyridine-3-carboxamide;
5-(4-((2-chloropyridin-3-yl)methoxy)phenyl)-2-oxo-6-(trifluoromethyl)-1,2-dihydropyridine-3-carboxamide;
2-oxo-5-(4-((5-(propylamino)pyridin-3-yl)methoxy)phenyl)-6-(trifluoromethyl)-1,2-dihydropyridine-3-carboxamide;
2-oxo-5-(4-(pyrimidin-2-ylmethoxy)phenyl)-6-(trifluoromethyl)-1,2-dihydropyridine-3-carboxamide;
5-(4-((2-morpholinopyrimidin-5-yl)methoxy)phenyl)-2-oxo-6-(trifluoromethyl)-1,2-dihydropyridine-3-carboxamide;
2-oxo-5-(4-(pyrazin-2-ylmethoxy)phenyl)-6-(trifluoromethyl)-1,2-dihydropyridine-3-carboxamide;
(R or S)-5-(4-(1-(5-cyanopyridin-3-yl)ethoxy)phenyl)-2-oxo-6-(trifluoromethyl)-1,2-dihydropyridine-3-carboxamide;
5-(4-((1-methyl-1H-pyrazol-5-yl)methoxy)phenyl)-2-oxo-6-(trifluoromethyl)-1,2-dihydropyridine-3-carboxamide;
5-(4-((4-methoxypyridin-2-yl)methoxy)phenyl)-2-oxo-6-(trifluoromethyl)-1,2-dihydropyridine-3-carboxamide;
5-(4-((8-morpholinopyridin-3-yl)methoxy)phenyl)-2-oxo-6-(trifluoromethyl)-1,2-dihydropyridine-3-carboxamide;
5-(4-((2-fluoropyridin-3-yl)methoxy)phenyl)-2-oxo-6-(trifluoromethyl)-1,2-dihydropyridine-3-carboxamide;
5-(4-((5-fluoro-2-methoxypyridin-4-yl)methoxy)phenyl)-2-oxo-6-(trifluoromethyl)-1,2-dihydropyridine-3-carboxamide;
2-oxo-5-(4-(quinoxalin-6-ylmethoxy)phenyl)-6-(trifluoromethyl)-1,2-dihydropyridine-3-carboxamide;
5-(4-((1-methyl-1H-indazol-4-yl)methoxy)phenyl)-2-oxo-6-(trifluoromethyl)-1,2-dihydropyridine-3-carboxamide;
5-(4-((4-(methylsulfonyl)benzyl)oxy)phenyl)-2-oxo-6-(trifluoromethyl)-1,2-dihydropyridine-3-carboxamide;
2-oxo-5-(4-(thiazol-5-ylmethoxy)phenyl)-6-(trifluoromethyl)-1,2-dihydropyridine-3-carboxamide;
5-(4-((4,8-dimethylpyridin-2-yl)methoxy)phenyl)-2-oxo-6-(trifluoromethyl)-1,2-dihydropyridine-3-carboxamide;
5-(4-((2-methoxypyridin-3-yl)methoxy)phenyl)-2-oxo-6-(trifluoromethyl)-1,2-dihydropyridine-3-carboxamide;
5-(4-(isoquinolin-7-ylmethoxy)phenyl)-2-oxo-6-(trifluoromethyl)-1,2-dihydropyridine-3-carboxamide;
2-oxo-5-(4-(quinolin-7-ylmethoxy)phenyl)-6-(trifluoromethyl)-1,2-dihydropyridine-3-carboxamide;
5-(4-((3-fluoropyridin-4-yl)methoxy)phenyl)-2-oxo-6-(trifluoromethyl)-1,2-dihydropyridine-3-carboxamide;
5-(4-((4,8-dimethylpyrimidin-2-yl)methoxy)phenyl)-2-oxo-6-(trifluoromethyl)-1,2-dihydropyridine-3-carboxamide;
5-(4-((1-methyl-1H-imidazol-2-yl)methoxy)phenyl)-2-oxo-6-(trifluoromethyl)-1,2-dihydropyridine-3-carboxamide;
5-(4-((2-morpholinopyridin-3-yl)methoxy)phenyl)-2-oxo-6-(trifluoromethyl)-1,2-dihydropyridine-3-carboxamide;
2-oxo-5-(4-(thiazol-2-ylmethoxy)phenyl)-6-(trifluoromethyl)-1,2-dihydropyridine-3-carboxamide;
5-(4-((3,4-dimethoxypyridin-2-yl)methoxy)phenyl)-2-oxo-6-(trifluoromethyl)-1,2-dihydropyridine-3-carboxamide;
5-(4-((1-methyl-1H-indazol-5-yl)methoxy)phenyl)-2-oxo-6-(trifluoromethyl)-1,2-dihydropyridine-3-carboxamide;
5-(4-((5-methoxypyridin-3-yl)methoxy)phenyl)-2-oxo-6-(trifluoromethyl)-1,2-dihydropyridine-3-carboxamide;
5-(4-((2,8-dimethylpyridin-4-yl)methoxy)phenyl)-2-oxo-6-(trifluoromethyl)-1,2-dihydropyridine-3-carboxamide;
5-(4-((8-chlorobenzo[d][1,3]dioxol-5-yl)methoxy)phenyl)-2-oxo-6-(trifluoromethyl)-1,2-dihydropyridine-3-carboxamide;
5-(4-((3,4-dihydro-2H-benzo[b][1,4]dioxepin-7-yl)methoxy)phenyl)-2-oxo-6-(trifluoromethyl)-1,2-dihydropyridine-3-carboxamide;
5-(4-((2-hydroxypyrimidin-5-yl)methoxy)phenyl)-2-oxo-6-(trifluoromethyl)-1,2-dihydropyridine-3-carboxamide;
5-(4-((2-(oxetan-3-ylmethoxy)pyrimidin-5-yl)methoxy)phenyl)-2-oxo-6-(trifluoromethyl)-1,2-dihydropyridine-3-carboxamide;
5-(4-((2-(2-hydroxyethoxy)pyrimidin-5-yl)methoxy)phenyl)-2-oxo-6-(trifluoromethyl)-1,2-dihydropyridine-3-carboxamide;
2-oxo-5-(4-((2-propoxypyrimidin-5-yl)methoxy)phenyl)-6-(trifluoromethyl)-1,2-dihydropyridine-3-carboxamide;
2-oxo-5-(4-((2-((tetrahydrofuran-3-yl)oxy)pyrimidin-5-yl)methoxy)phenyl)-6-(trifluoromethyl)-1,2-dihydropyridine-3-carboxamide;
5-(4-((2-(cyclopentyloxy)pyrimidin-5-yl)methoxy)phenyl)-2-oxo-6-(trifluoromethyl)-1,2-dihydropyridine-3-carboxamide;
5-(4-((2-(oxetan-2-ylmethoxy)pyrimidin-5-yl)methoxy)phenyl)-2-oxo-6-(trifluoromethyl)-1,2-dihydropyridine-3-carboxamide;
2-oxo-5-(4-((2-((tetrahydrofuran-3-yl)methoxy)pyrimidin-5-yl)methoxy)phenyl)-6-(trifluoromethyl)-1,2-dihydropyridine-3-carboxamide;
5-(4-((2-(methylamino)pyrimidin-5-yl)methoxy)phenyl)-2-oxo-6-(trifluoromethyl)-1,2-dihydropyridine-3-carboxamide;
5-(4-((2-(cyclopentylamino)pyrimidin-5-yl)methoxy)phenyl)-2-oxo-6-(trifluoromethyl)-1,2-dihydropyridine-3-carboxamide;
5-(4-((2-(dimethylamino)pyrimidin-5-yl)methoxy)phenyl)-2-oxo-6-(trifluoromethyl)-1,2-dihydropyridine-3-carboxamide;
5-(4-((2-((cyclopropylmethyl)amino)pyrimidin-5-yl)methoxy)phenyl)-2-oxo-6-(trifluoromethyl)-1,2-dihydropyridine-3-carboxamide;
5-(4-((2-((2-hydroxyethylamino)pyrimidin-5-yl)methoxy)phenyl)-2-oxo-6-(trifluoromethyl)-1,2-dihydropyridine-3-carboxamide;
5-(4-((2-(cyclopropylamino)pyrimidin-5-yl)methoxy)phenyl)-2-oxo-6-(trifluoromethyl)-1,2-dihydropyridine-3-carboxamide;
5-(4-((2-(heptylamino)pyrimidin-5-yl)methoxy)phenyl)-2-oxo-6-(trifluoromethyl)-1,2-dihydropyridine-3-carboxamide;
5-(4-((2-(oxetan-3-ylamino)pyrimidin-5-yl)methoxy)phenyl)-2-oxo-6-(trifluoromethyl)-1,2-dihydropyridine-3-carboxamide;
2-oxo-5-(4-((2-((tetrahydrofuran-3-yl)amino)pyrimidin-5-yl)methoxy)phenyl)-6-(trifluoromethyl)-1,2-dihydropyridine-3-carboxamide;
5-(4-((6-(cyclopentylamino)pyridin-3-yl)methoxy)phenyl)-2-oxo-6-(trifluoromethyl)-1,2-dihydropyridine-3-carboxamide;
5-(4-((6-((cyclopropylmethylamino)pyridin-3-yl)methoxy)phenyl)-2-oxo-6-(trifluoromethyl)-1,2-dihydropyridine-3-carboxamide;
5-(4-((6-(3,3-difluoroazetidin-1-yl)pyridin-3-yl)methoxy)phenyl)-2-oxo-6-(trifluoromethyl)-1,2-dihydropyridine-3-carboxamide;
5-(4-((6-(3-methoxyazetidin-1-yl)pyridin-3-yl)methoxy)phenyl)-2-oxo-6-(trifluoromethyl)-1,2-dihydropyridine-3-carboxamide;
5-(4-((2-cyclopropylpyrimidin-5-yl)methoxy)phenyl)-2-oxo-6-(trifluoromethyl)-1,2-dihydropyridine-3-carboxamide;
5-(4-((2-(cyclopentylmethyl)pyrimidin-5-yl)methoxy)phenyl)-2-oxo-6-(trifluoromethyl)-1,2-dihydropyridine-3-carboxamide;
5-(4-((2-neopentylpyrimidin-5-yl)methoxy)phenyl)-2-oxo-6-(trifluoromethyl)-1,2-dihydropyridine-3-carboxamide;
5-(4-((4-(cyclopentylamino)pyrimidin-5-yl)methoxy)phenyl)-2-oxo-6-(trifluoromethyl)-1,2-dihydropyridine-3-carboxamide;
5-(4-((3-(cyclopropylmethyl)-3H-imidazo[4,5-b]pyridin-6-yl)methoxy)phenyl)-2-oxo-6-(trifluoromethyl)-1,2-dihydropyridine-3-carboxamide;
5-(4-((8-chloropyridin-3-yl)methoxy)phenyl)-2-oxo-6-(trifluoromethyl)-1,2-dihydropyridine-3-carboxamide;
2-oxo-5-(4-((pyridin-3-yloxy)methyl)phenyl)-6-(trifluoromethyl)-1,2-dihydropyridine-3-carboxamide;
5-(4-((5-(furan-3-yl)pyridin-3-yl)methoxy)phenyl)-2-oxo-6-(trifluoromethyl)-1,2-dihydropyridine-3-carboxamide;
5-(4-((5-morpholinopyridin-3-yl)methoxy)phenyl)-2-oxo-6-(trifluoromethyl)-1,2-dihydropyridine-3-carboxamide;
2-oxo-5-(4-((5-(tetrahydrofuran-3-yl)pyridin-3-yl)methoxy)phenyl)-6-(trifluoromethyl)-1,2-dihydropyridine-3-carboxamide;
5-(4-((5-(3-hydroxyoxetan-3-yl)pyridin-3-yl)methoxy)phenyl)-2-oxo-6-(trifluoromethyl)-1,2-dihydropyridine-3-carboxamide;
5-(4-((5-(3-fluorooxetan-3-yl)pyridin-3-yl)methoxy)phenyl)-2-oxo-6-(trifluoromethyl)-1,2-dihydropyridine-3-carboxamide;
2-oxo-5-(4-((3-(pyridin-3-yl)oxetan-3-yl)oxy)phenyl)-6-(trifluoromethyl)-1,2-dihydropyridine-3-carboxamide;
5-(4-(cyclopropyl(pyridin-3-yl)methoxy)phenyl)-2-oxo-6-(trifluoromethyl)-1,2-dihydropyridine-3-carboxamide;
2-oxo-5-(4-((1-(pyridin-3-yl)pent-4-en-1-yl)oxy)phenyl)-6-(trifluoromethyl)-1,2-dihydropyridine-3-carboxamide;
5-(4-(2-methoxy-1-(pyridin-3-yl)ethoxy)phenyl)-2-oxo-6-(trifluoromethyl)-1,2-dihydropyridine-3-carboxamide;
5-(4-(2-hydroxy-1-(pyrazin-2-yl)ethoxy)phenyl)-2-oxo-6-(trifluoromethyl)-1,2-dihydropyridine-3-carboxamide;
5-(4-((4-acetylmorpholin-2-yl)methoxy)phenyl)-2-oxo-6-(trifluoromethyl)-1,2-dihydropyridine-3-carboxamide;
5-(4-(morpholin-2-ylmethoxy)phenyl)-2-oxo-6-(trifluoromethyl)-1,2-dihydropyridine-3-carboxamide;
tert-butyl 2-((4-(5-carbamoyl-6-oxo-2-(trifluoromethyl)-1,8-dihydropyridin-3-yl)phenoxy)methyl)morpholine-4-carboxylate;
5-(4-((4-(isopropylsulfonyl)morpholin-2-yl)methoxy)phenyl)-2-oxo-6-(trifluoromethyl)-1,2-dihydropyridine-3-carboxamide;
5-(4-((4-(2,2-difluoroethyl)morpholin-2-yl)methoxy)phenyl)-2-oxo-6-(trifluoromethyl)-1,2-dihydropyridine-3-carboxamide;
5-(4-((4-(2-methoxyethyl)morpholin-2-yl)methoxy)phenyl)-2-oxo-6-(trifluoromethyl)-1,2-dihydropyridine-3-carboxamide;
5-(4-((4-(cyclopropylmethyl)morpholin-2-yl)methoxy)phenyl)-2-oxo-6-(trifluoromethyl)-1,2-dihydropyridine-3-carboxamide;
5-(4-((1,4-oxazepan-2-yl)methoxy)phenyl)-2-oxo-6-(trifluoromethyl)-1,2-dihydropyridine-3-carboxamide;
5-(4-((4-(oxetan-3-yl)-1,4-oxazepan-2-yl)methoxy)phenyl)-2-oxo-6-(trifluoromethyl)-1,2-dihydropyridine-3-carboxamide;
5-(4-((4-(isopropylsulfonyl)-1,4-oxazepan-2-yl)methoxy)phenyl)-2-oxo-6-(trifluoromethyl)-1,2-dihydropyridine-3-carboxamide;
5-(4-((4-(isobutylsulfonyl)-1,4-oxazepan-2-yl)methoxy)phenyl)-2-oxo-6-(trifluoromethyl)-1,2-dihydropyridine-3-carboxamide;
5-(4-(2-morpholino-2-oxoethoxy)phenyl)-2-oxo-6-(trifluoromethyl)-1,2-dihydropyridine-3-carboxamide;
5-(4-(2-(butylamino)-2-oxoethoxy)phenyl-2-oxo-6-(trifluoromethyl)-1,2-dihydropyridine-3-carboxamide;
2-oxo-5-(4-(2-oxo-2-(pyridin-3-ylamino)ethoxy)phenyl)-6-(trifluoromethyl)-1,2-dihydropyridine-3-carboxamide;
5-(4-((4-methoxytetrahydro-2H-pyran-2-yl)methoxy)phenyl)-2-oxo-6-(trifluoromethyl)-1,2-dihydropyridine-3-carboxamide;
5-(4-(((2S,5S)-5-(hydroxymethyl)-1,4-dioxan-2-yl)methoxy)phenyl)-2-oxo-6-(trifluoromethyl)-1,2-dihydropyridine-3-carboxamide;
5-(4-((2,8-dioxa-5-azaspiro[3.5]nonan-6-yl)methoxy)phenyl)-2-oxo-6-(trifluoromethyl)-1,2-dihydropyridine-3-carboxamide;
5-(4-((5-methyl-2,8-dioxa-5-azaspiro[3.5]nonan-6-yl)methoxy)phenyl)-2-oxo-6-(trifluoromethyl)-1,2-dihydropyridine-3-carboxamide;
2-oxo-6-(trifluoromethyl)-5-(4-((4,5,5-trimethylmorpholin-3-yl)methoxy)phenyl)-1,2-dihydropyridine-3-carboxamide;
5-(4-(((5S)-4,5-dimethylmorpholin-3-yl)methoxy)phenyl)-2-oxo-6-(trifluoromethyl)-1,2-dihydropyridine-3-carboxamide;
5-(4-(((3S,5S)-5-cyclopropyl-4-methylmorpholin-3-yl)methoxy)phenyl)-2-oxo-6-(trifluoromethyl)-1,2-dihydropyridine-3-carboxamide;
5-(4-(((3R,5S)-5-cyclopropyl-4-methylmorpholin-3-yl)methoxy)phenyl)-2-oxo-6-(trifluoromethyl)-1,2-dihydropyridine-3-carboxamide;
5-(4-(((5S)-5-isopropyl-4-methylmorpholin-3-yl)methoxy)phenyl)-2-oxo-6-(trifluoromethyl)-1,2-dihydropyridine-3-carboxamide;
5-(4-((5-bromopyridin-3-yl)methoxy)phenyl)-2-oxo-6-(trifluoromethyl)-1,2-dihydropyridine-3-carboxamide;
5-(4-((5-chloropyridin-3-yl)methoxy)phenyl)-2-oxo-6-(trifluoromethyl)-1,2-dihydropyridine-3-carboxamide;
2-oxo-5-(4-(pyridazin-3-ylmethoxy)phenyl)-6-(trifluoromethyl)-1,2-dihydropyridine-3-carboxamide;
2-oxo-5-(4-(pyrimidin-4-ylmethoxy)phenyl)-6-(trifluoromethyl)-1,2-dihydropyridine-3-carboxamide;
5-(4-((8-chloropyrazin-2-yl)methoxy)phenyl)-2-oxo-6-(trifluoromethyl)-1,2-dihydropyridine-3-carboxamide;
5-(4-((1-ethyl-1H-pyrazol-5-yl)methoxy)phenyl)-2-oxo-6-(trifluoromethyl)-1,2-dihydropyridine-3-carboxamide;
5-(4-((1-(2-methoxyethyl)-1H-pyrazol-4-yl)methoxy)phenyl)-2-oxo-6-(trifluoromethyl)-1,2-dihydropyridine-3-carboxamide;
5-(4-((1-(2-methoxyethyl)-1H-pyrazol-5-yl)methoxy)phenyl)-2-oxo-6-(trifluoromethyl)-1,2-dihydropyridine-3-carboxamide;
5-(4-((1-methyl-1H-pyrazol-4-yl)methoxy)phenyl)-2-oxo-6-(trifluoromethyl)-1,2-dihydropyridine-3-carboxamide;
5-(4-((1-(cyclopropylmethyl)-1H-pyrazol-4-yl)methoxy)phenyl)-2-oxo-6-(trifluoromethyl)-1,2-dihydropyridine-3-carboxamide;
5-(4-((1-ethyl-1H-pyrazol-4-yl)methoxy)phenyl)-2-oxo-6-(trifluoromethyl)-1,2-dihydropyridine-3-carboxamide;
5-(4-(isoxazol-3-ylmethoxy)phenyl)-2-oxo-6-(trifluoromethyl)-1,2-dihydropyridine-3-carboxamide;
5-(4-((5-methylisoxazol-3-yl)methoxy)phenyl)-2-oxo-6-(trifluoromethyl)-1,2-dihydropyridine-3-carboxamide;
5-(4-(oxetan-2-ylmethoxy)phenyl)-2-oxo-6-(trifluoromethyl)-1,2-dihydropyridine-3-carboxamide;
5-(4-((3-fluorooxetan-3-yl)methoxy)phenyl)-2-oxo-6-(trifluoromethyl)-1,2-dihydropyridine-3-carboxamide;
5-(4-((3-methyl)oxetan-3-yl)methoxy)phenyl)-2-oxo-6-(trifluoromethyl)-1,2-dihydropyridine-3-carboxamide;
5-(4-((3-ethyl)oxetan-3-yl)methoxy)phenyl)-2-oxo-6-(trifluoromethyl)-1,2-dihydropyridine-3-carboxamide;
5-(4-((3-(cyanomethyl)oxetan-3-yl)methoxy)phenyl)-2-oxo-6-(trifluoromethyl)-1,2-dihydropyridine-3-carboxamide;
5-(4-((2-oxaspiro[3.3]heptan-6-yl)oxy)phenyl)-2-oxo-6-(trifluoromethyl)-1,2-dihydropyridine-3-carboxamide;
2-oxo-5-(4-((tetrahydrofuran-3-yl)methoxy)phenyl)-6-(trifluoromethyl)-1,2-dihydropyridine-3-carboxamide;
2-oxo-5-(4-((tetrahydrofuran-2-yl)methoxy)phenyl)-6-(trifluoromethyl)-1,2-dihydropyridine-3-carboxamide;
5-(4-((3-methyl-2-oxooxazolidin-5-yl)methoxy)phenyl)-2-oxo-6-(trifluoromethyl)-1,2-dihydropyridine-3-carboxamide;
5-(4-((3-ethyl-2-oxooxazolidin-5-yl)methoxy)phenyl)-2-oxo-6-(trifluoromethyl)-1,2-dihydropyridine-3-carboxamide;
5-(4-((3-methyl-2-oxooxazolidin-4-yl)methoxy)phenyl)-2-oxo-6-(trifluoromethyl)-1,2-dihydropyridine-3-carboxamide;
2-oxo-5-(4-((tetrahydro-2H-pyran-2-yl)methoxy)phenyl)-6-(trifluoromethyl)-1,2-dihydropyridine-3-carboxamide;
5-(4-((1,4-dioxan-2-yl)methoxy)phenyl)-2-oxo-6-(trifluoromethyl)-1,2-dihydropyridine-3-carboxamide;
5-(4-((1,4-dioxan-2-yl)methoxy)-3-fluorophenyl)-2-oxo-6-(trifluoromethyl)-1,2-dihydropyridine-3-carboxamide;
5-(4-((1,4-dioxan-2-yl)methoxy)-3-chlorophenyl)-2-oxo-6-(trifluoromethyl)-1,2-dihydropyridine-3-carboxamide;
5-(4-((1,4-dioxan-2-yl)methoxy)-3-cyanophenyl)-2-oxo-6-(trifluoromethyl)-1,2-dihydropyridine-3-carboxamide;
5-(4-((1,4-dioxan-2-yl)methoxy)-3-methylphenyl)-2-oxo-6-(trifluoromethyl)-1,2-dihydropyridine-3-carboxamide;
5-(4-((1,4-dioxan-2-yl)methoxy)-3-cyclopropylphenyl)-2-oxo-6-(trifluoromethyl)-1,2-dihydropyridine-3-carboxamide;
5-(4-((1,4-dioxan-2-yl)methoxy)-3-methoxyphenyl)-2-oxo-6-(trifluoromethyl)-1,2-dihydropyridine-3-carboxamide;
5-(4-((1,4-dioxan-2-yl)methoxy)-2-fluorophenyl)-2-oxo-6-(trifluoromethyl)-1,2-dihydropyridine-3-carboxamide;
5-(4-(morpholin-3-ylmethoxy)phenyl)-2-oxo-6-(trifluoromethyl)-1,2-dihydropyridine-3-carboxamide;
5-(4-((4-methylmorpholin-3-yl)methoxy)phenyl)-2-oxo-6-(trifluoromethyl)-1,2-dihydropyridine-3-carboxamide;
5-(4-((4-cyclopropylmorpholin-3-yl)methoxy)phenyl)-2-oxo-6-(trifluoromethyl)-1,2-dihydropyridine-3-carboxamide;
5-(4-((4-ethylmorpholin-3-yl)methoxy)phenyl)-2-oxo-6-(trifluoromethyl)-1,2-dihydropyridine-3-carboxamide;
2-oxo-5-(4-((5-oxomorpholin-3-yl)methoxy)phenyl)-6-(trifluoromethyl)-1,2-dihydropyridine-3-carboxamide;
5-(4-((4-methyl-5-oxomorpholin-3-yl)methoxy)phenyl)-2-oxo-6-(trifluoromethyl)-1,2-dihydropyridine-3-carboxamide;
5-(4-((1-(methylsulfonyl)cyclopropyl)methoxy)phenyl)-2-oxo-6-(trifluoromethyl)-1,2-dihydropyridine-3-carboxamide;
5-(4-((1-(cyclopropylsulfonyl)cyclopropyl)methoxy)phenyl)-2-oxo-6-(trifluoromethyl)-1,2-dihydropyridine-3-carboxamide;
5-(4-(2-methoxyethoxy)phenyl)-2-oxo-6-(trifluoromethyl)-1,2-dihydropyridine-3-carboxamide;
5-(4-(2-hydroxyethoxy)phenyl)-2-oxo-6-(trifluoromethyl)-1,2-dihydropyridine-3-carboxamide;
5-(4-(2-(methylsulfonyl)ethoxy)phenyl)-2-oxo-6-(trifluoromethyl)-1,2-dihydropyridine-3-carboxamide;
2-oxo-5-(4-(2-(2-oxooxazolidin-3-yl)ethoxy)phenyl)-6-(trifluoromethyl)-1,2-dihydropyridine-3-carboxamide;
5-(4-(2-(1H-imidazol-1-yl)ethoxy)phenyl)-2-oxo-6-(trifluoromethyl)-1,2-dihydropyridine-3-carboxamide;
5-(4-(2-morpholinoethoxy)phenyl)-2-oxo-6-(trifluoromethyl)-1,2-dihydropyridine-3-carboxamide;
5-(4-(2-hydroxy-3-morpholinopropoxy)phenyl)-2-oxo-6-(trifluoromethyl)-1,2-dihydropyridine-3-carboxamide;
5-(4-((1,3-dimethyl-1H-pyrazol-5-yl)methoxy)phenyl)-2-oxo-6-(trifluoromethyl)-1,2-dihydropyridine-3-carboxamide;
5-(4-((3-ethyl-1-methyl-1H-pyrazol-5-yl)methoxy)phenyl)-2-oxo-6-(trifluoromethyl)-1,2-dihydropyridine-3-carboxamide;
5-(4-((3-cyclopropyl-1-methyl-1H-pyrazol-5-yl)methoxy)phenyl)-2-oxo-6-(trifluoromethyl)-1,2-dihydropyridine-3-carboxamide;
2-oxo-6-(trifluoromethyl)-5-(4-((1,3,4-trimethyl-1H-pyrazol-5-yl)methoxy)phenyl)-1,2-dihydropyridine-3-carboxamide;
5-(4-((1-(cyclopropylmethyl)-1H-pyrazol-5-yl)methoxy)phenyl)-2-oxo-6-(trifluoromethyl)-1,2-dihydropyridine-3-carboxamide;
5-(4-(oxazol-4-ylmethoxy)phenyl)-2-oxo-6-(trifluoromethyl)-1,2-dihydropyridine-3-carboxamide;
5-(4-(oxazol-5-ylmethoxy)phenyl)-2-oxo-6-(trifluoromethyl)-1,2-dihydropyridine-3-carboxamide;
5-(4-((5-cyclopropylisoxazol-3-yl)methoxy)phenyl)-2-oxo-6-(trifluoromethyl)-1,2-dihydropyridine-3-carboxamide;
5-(4-((5-methylisoxazol-4-yl)methoxy)phenyl)-2-oxo-6-(trifluoromethyl)-1,2-dihydropyridine-3-carboxamide;
5-(4-((5-methyl-3-(pyridin-4-yl)isoxazol-4-yl)methoxy)phenyl)-2-oxo-6-(trifluoromethyl)-1,2-dihydropyridine-3-carboxamide;
5-(4-((2-methylthiazol-5-yl)methoxy)phenyl)-2-oxo-6-(trifluoromethyl)-1,2-dihydropyridine-3-carboxamide;
5-(4-((4-methylthiazol-5-yl)methoxy)phenyl)-2-oxo-6-(trifluoromethyl)-1,2-dihydropyridine-3-carboxamide;
5-(4-((4-methyl-1,2,3-thiadiazol-5-yl)methoxy)phenyl)-2-oxo-6-(trifluoromethyl)-1,2-dihydropyridine-3-carboxamide;
5-(4-((4-methylpyridin-3-yl)methoxy)phenyl)-2-oxo-6-(trifluoromethyl)-1,2-dihydropyridine-3-carboxamide;
5-(4-((4-cyclopropylpyridin-3-yl)methoxy)phenyl)-2-oxo-6-(trifluoromethyl)-1,2-dihydropyridine-3-carboxamide;
5-(4-((4-methoxypyridin-3-yl)methoxy)phenyl)-2-oxo-6-(trifluoromethyl)-1,2-dihydropyridine-3-carboxamide;
5-(4-((4-ethoxypyridin-3-yl)methoxy)phenyl)-2-oxo-6-(trifluoromethyl)-1,2-dihydropyridine-3-carboxamide;
5-(4-((5-methylpyridin-3-yl)methoxy)phenyl)-2-oxo-6-(trifluoromethyl)-1,2-dihydropyridine-3-carboxamide;
5-(4-((5-cyclopropylpyridin-3-yl)methoxy)phenyl)-2-oxo-6-(trifluoromethyl)-1,2-dihydropyridine-3-carboxamide;
5-(4-((4-isopropoxypyridin-3-yl)methoxy)phenyl)-2-oxo-6-(trifluoromethyl)-1,2-dihydropyridine-3-carboxamide;
5-(4-((4-methylpyrimidin-5-yl)methoxy)phenyl)-2-oxo-6-(trifluoromethyl)-1,2-dihydropyridine-3-carboxamide;
5-(4-((4-cyclopropylpyrimidin-5-yl)methoxy)phenyl)-2-oxo-6-(trifluoromethyl)-1,2-dihydropyridine-3-carboxamide;
5-(4-((8-methylpyrazin-2-yl)methoxy)phenyl)-2-oxo-6-(trifluoromethyl)-1,2-dihydropyridine-3-carboxamide;
5-(4-((8-cyclopropylpyrazin-2-yl)methoxy)phenyl)-2-oxo-6-(trifluoromethyl)-1,2-dihydropyridine-3-carboxamide;
5-(4-(imidazo[1,2-a]pyridin-2-ylmethoxy)phenyl)-2-oxo-6-(trifluoromethyl)-1,2-dihydropyridine-3-carboxamide;
5-(4-(imidazo[1,2-a]pyridin-3-ylmethoxy)phenyl)-2-oxo-6-(trifluoromethyl)-1,2-dihydropyridine-3-carboxamide;
2-oxo-5-(4-((4,5,6,7-tetrahydrobenzo[d]isoxazol-3-yl)methoxy)phenyl)-6-(trifluoromethyl)-1,2-dihydropyridine-3-carboxamide;
5-(4-((2,3-dihydrobenzofuran-2-yl)methoxy)phenyl)-2-oxo-6-(trifluoromethyl)-1,2-dihydropyridine-3-carboxamide;
5-(4-((1-methyl-1,4,5,7-tetrahydropyrano[3,4-c]pyrazol-4-yl)methoxy)phenyl)-2-oxo-6-(trifluoromethyl)-1,2-dihydropyridine-3-carboxamide;
5-(4-((2-methyl-2,4,5,7-tetrahydropyrano[3,4-c]pyrazol-4-yl)methoxy)phenyl)-2-oxo-6-(trifluoromethyl)-1,2-dihydropyridine-3-carboxamide;
5-(4-((1-isopropyl-1,4,5,7-tetrahydropyrano[3,4-c]pyrazol-4-yl)methoxy)phenyl)-2-oxo-6-(trifluoromethyl)-1,2-dihydropyridine-3-carboxamide;
5-(4-((2-isopropyl-2,4,5,7-tetrahydropyrano[3,4-c]pyrazol-4-yl)methoxy)phenyl)-2-oxo-6-(trifluoromethyl)-1,2-dihydropyridine-3-carboxamide;
5-(4-(chroman-2-ylmethoxy)phenyl)-2-oxo-6-(trifluoromethyl)-1,2-dihydropyridine-3-carboxamide;
tert-butyl 3-((4-(5-carbamoyl-6-oxo-2-(trifluoromethyl)-1,6-dihydropyridin-3-yl)phenoxy)methyl)azetidine-1-carboxylate;
tert-butyl ((1r,3r)-3-((4-(5-carbamoyl-6-oxo-2-(trifluoromethyl)-1,6-dihydropyridin-3-yl)phenoxy)methyl)cyclobutyl)carbamate;
(S)-5-(4-((5,5-dimethyltetrahydrofuran-2-yl)methoxy)phenyl)-2-oxo-6-(trifluoromethyl)-1,2-dihydropyridine-3-carboxamide;
tert-butyl 6-((4-(5-carbamoyl-6-oxo-2-(trifluoromethyl)-1,6-dihydropyridin-3-yl)phenoxy)methyl)-3-azabicyclo[3.1.0]hexane-3-carboxylate;
tert-butyl 3-((4-(5-carbamoyl-6-oxo-2-(trifluoromethyl)-1,6-dihydropyridin-3-yl)phenoxy)methyl)pyrrolidine-1-carboxylate;
tert-butyl 3-((4-(5-carbamoyl-6-oxo-2-(trifluoromethyl)-1,6-dihydropyridin-3-yl)phenoxy)methyl)-4-methylpyrrolidine-1-carboxylate;
5-(4-((1-benzyl-3-cyanopyrrolidin-3-yl)methoxy)phenyl)-2-oxo-6-(trifluoromethyl)-1,2-dihydropyridine-3-carboxamide;
tert-butyl (2S,4R)-2-((4-(5-carbamoyl-6-oxo-2-(trifluoromethyl)-1,6-dihydropyridin-3-ylphenoxy)methyl)-4-fluoropyrrolidine-1-carboxylate;
tert-butyl (2S,4S)-2-((4-(5-carbamoyl-6-oxo-2-(trifluoromethyl)-1,6-dihydropyridin-3-ylphenoxy)methyl)-4-fluoropyrrolidine-1-carboxylate;
5-(4-((3-isopropyl-2-oxooxazolidin-5-yl)methoxy)phenyl)-2-oxo-6-(trifluoromethyl)-1,2-dihydropyridine-3-carboxamide;
5-(4-((3-(cyclopropylmethyl)-2-oxooxazolidin-5-yl)methoxy)phenyl)-2-oxo-6-(trifluoromethyl)-1,2-dihydropyridine-3-carboxamide;
5-(4-((3-ethyl-2-oxooxazolidin-4-yl)methoxy)phenyl)-2-oxo-6-(trifluoromethyl)-1,2-dihydropyridine-3-carboxamide;
5-(4-((3-isopropyl-2-oxooxazolidin-4-yl)methoxy)phenyl)-2-oxo-6-(trifluoromethyl)-1,2-dihydropyridine-3-carboxamide;
5-(4-((3-(cyclopropylmethyl)-2-oxooxazolidin-4-yl)methoxy)phenyl)-2-oxo-6-(trifluoromethyl)-1,2-dihydropyridine-3-carboxamide;
5-(4-((7-oxabicyclo[2.2.1]heptan-2-yl)methoxy)phenyl)-2-oxo-6-(trifluoromethyl)-1,2-dihydropyridine-3-carboxamide;
5-(4-((3,8-dioxabicyclo[3.2.1]octan-2-yl)methoxy)phenyl)-2-oxo-6-(trifluoromethyl)-1,2-dihydropyridine-3-carboxamide;
5-(4-((1,4-dioxepan-6-yl)methoxy)phenyl)-2-oxo-6-(trifluoromethyl)-1,2-dihydropyridine-3-carboxamide;
5-(4-((6-methyl-1,4-dioxepan-6-yl)methoxy)phenyl)-2-oxo-6-(trifluoromethyl)-1,2-dihydropyridine-3-carboxamide;
5-(4-((5-methyl-1,4-dioxan-2-yl)methoxy)phenyl)-2-oxo-6-(trifluoromethyl)-1,2-dihydropyridine-3-carboxamide;
5-(4-(((2R,6R)-6-cyclopropyl-1,4-dioxan-2-yl)methoxy)phenyl)-2-oxo-6-(trifluoromethyl)-1,2-dihydropyridine-3-carboxamide;
5-(4-(((2R,6S)-6-cyclopropyl-1,4-dioxan-2-yl)methoxy)phenyl)-2-oxo-6-(trifluoromethyl)-1,2-dihydropyridine-3-carboxamide;
5-(4-(((2R,6R)-6-methyl-1,4-dioxan-2-yl)methoxy)phenyl)-2-oxo-6-(trifluoromethyl)-1,2-dihydropyridine-3-carboxamide;
5-(4-(((2S,6R)-6-methyl-1,4-dioxan-2-yl)methoxy)phenyl)-2-oxo-6-(trifluoromethyl)-1,2-dihydropyridine-3-carboxamide;
5-(4-(((2S,6S)-6-methyl-1,4-dioxan-2-yl)methoxy)phenyl)-2-oxo-6-(trifluoromethyl)-1,2-dihydropyridine-3-carboxamide;
5-(4-(((2R,6S)-6-methyl-1,4-dioxan-2-yl)methoxy)phenyl)-2-oxo-6-(trifluoromethyl)-1,2-dihydropyridine-3-carboxamide;
5-(4-((6-cyclopentyl-1,4-dioxan-2-yl)methoxy)phenyl)-2-oxo-6-(trifluoromethyl)-1,2-dihydropyridine-3-carboxamide;
5-(4-((4,7-dioxaspiro[2.5]octan-5-yl)methoxy)phenyl)-2-oxo-6-(trifluoromethyl)-1,2-dihydropyridine-3-carboxamide;
5-(4-((5,5-difluorotetrahydro-2H-pyran-3-yl)methoxy)phenyl)-2-oxo-6-(trifluoromethyl)-1,2-dihydropyridine-3-carboxamide;
5-(4-((4,7-dioxaspiro[2.5]octan-6-yl)methoxy)phenyl)-2-oxo-6-(trifluoromethyl)-1,2-dihydropyridine-3-carboxamide;
5-(4-((5,5-dimethyl-1,4-dioxan-2-yl)methoxy)phenyl)-2-oxo-6-(trifluoromethyl)-1,2-dihydropyridine-3-carboxamide;
5-(4-((5-(fluoromethyl)-1,4-dioxan-2-yl)methoxy)phenyl)-2-oxo-6-(trifluoromethyl)-1,2-dihydropyridine-3-carboxamide;
5-(4-((5-(difluoromethyl)-1,4-dioxan-2-yl)methoxy)phenyl)-2-oxo-6-(trifluoromethyl)-1,2-dihydropyridine-3-carboxamide;
5-(4-((6-(difluoromethyl)-1,4-dioxan-2-yl)methoxy)phenyl)-2-oxo-6-(trifluoromethyl)-1,2-dihydropyridine-3-carboxamide;
5-(4-((6-(fluoromethyl)-1,4-dioxan-2-yl)methoxy)phenyl)-2-oxo-6-(trifluoromethyl)-1,2-dihydropyridine-3-carboxamide;
5-(4-((6-(1-methoxycyclopropyl)-1,4-dioxan-2-yl)methoxy)phenyl)-2-oxo-6-(trifluoromethyl)-1,2-dihydropyridine-3-carboxamide;
5-(4-((6-(2-hydroxypropan-2-yl)-1,4-dioxan-2-yl)methoxy)phenyl)-2-oxo-6-(trifluoromethyl)-1,2-dihydropyridine-3-carboxamide;
5-(4-((6-(1-methylcyclopropyl)-1,4-dioxan-2-yl)methoxy)phenyl)-2-oxo-6-(trifluoromethyl)-1,2-dihydropyridine-3-carboxamide;
5-(4-((6-(1,1-difluoroethyl)-1,4-dioxan-2-yl)methoxy)phenyl)-2-oxo-6-(trifluoromethyl)-1,2-dihydropyridine-3-carboxamide;
5-(4-((6-(ethoxymethyl)-1,4-dioxan-2-yl)methoxy)phenyl)-2-oxo-6-(trifluoromethyl)-1,2-dihydropyridine-3-carboxamide;
5-(4-((6-((cyclopropylmethoxy)methyl)-1,4-dioxan-2-yl)methoxy)phenyl)-2-oxo-6-(trifluoromethyl)-1,2-dihydropyridine-3-carboxamide;
5-(4-((6-(((1-fluorocyclopropyl)methoxy)methyl)-1,4-dioxan-2-yl)methoxy)phenyl)-2-oxo-6-(trifluoromethyl)-1,2-dihydropyridine-3-carboxamide;
5-(4-((6-((3,3-difluorocyclobutoxy)methyl)-1,4-dioxan-2-yl)methoxy)phenyl)-2-oxo-6-(trifluoromethyl)-1,2-dihydropyridine-3-carboxamide;
5-(4-((6-((2,2-difluoroethoxy)methyl)-1,4-dioxan-2-yl)methoxy)phenyl)-2-oxo-6-(trifluoromethyl)-1,2-dihydropyridine-3-carboxamide;
5-(4-((6-((2-methoxy-2-methylpropoxy)methyl)-1,4-dioxan-2-yl)methoxy)phenyl)-2-oxo-6-(trifluoromethyl)-1,2-dihydropyridine-3-carboxamide;
2-oxo-5-(4-((6-((trifluoromethoxy)methyl)-1,4-dioxan-2-yl)methoxy)phenyl)-6-(trifluoromethyl)-1,2-dihydropyridine-3-carboxamide;
5-(4-((6-((1H-pyrrol-1-yl)methyl)-1,4-dioxan-2-yl)methoxy)phenyl)-2-oxo-6-(trifluoromethyl)-1,2-dihydropyridine-3-carboxamide;
5-(4-((6-((1H-pyrazol-1-yl)methyl)-1,4-dioxan-2-yl)methoxy)phenyl)-2-oxo-6-(trifluoromethyl)-1,2-dihydropyridine-3-carboxamide;
5-(4-((6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide;
5-(4-((3-cyclopropyl-6,7-dihydro-4H-[1,2,3]triazolo[5,1-c][1,4]oxazin-6-yl)methoxy)phenyl)-2-oxo-6-(trifluoromethyl)-1,2-dihydropyridine-3-carboxamide;
5-(4-((3-cyclopropyl-6,7-dihydro-4H-[1,2,3]triazolo[5,1-c][1,4]oxazin-7-yl)methoxy)phenyl)-2-oxo-6-(trifluoromethyl)-1,2-dihydropyridine-3-carboxamide;
5-(4-((6-(2-methoxypropan-2-yl)-1,4-dioxan-2-yl)methoxy)phenyl)-2-oxo-6-(trifluoromethyl)-1,2-dihydropyridine-3-carboxamide;
5-(4-((6-(3-fluoropentan-3-yl)-1,4-dioxan-2-yl)methoxy)phenyl)-2-oxo-6-(trifluoromethyl)-1,2-dihydropyridine-3-carboxamide;
5-(4-((6-(hydroxymethyl)-1,4-dioxan-2-yl)methoxy)phenyl)-2-oxo-6-(trifluoromethyl)-1,2-dihydropyridine-3-carboxamide;
5-(4-((6-((3-fluoroazetidin-1-ylmethyl)-1,4-dioxan-2-yl)methoxy)phenyl)-2-oxo-6-(trifluoromethyl)-1,2-dihydropyridine-3-carboxamide;
5-(4-((6-((3,3-difluoroazetidin-1-yl)methyl)-1,4-dioxan-2-yl)methoxy)phenyl)-2-oxo-6-(trifluoromethyl)-1,2-dihydropyridine-3-carboxamide;
5-(4-((6-((3-methoxyazetidin-1-yl)methyl)-1,4-dioxan-2-yl)methoxy)phenyl)-2-oxo-6-(trifluoromethyl)-1,2-dihydropyridine-3-carboxamide;
5-(4-((2-fluoro-1,4-dioxan-2-yl)methoxy)phenyl)-2-oxo-6-(trifluoromethyl)-1,2-dihydropyridine-3-carboxamide;
5-(4-((1,4-dioxan-2-yl)difluoromethoxy)phenyl)-2-oxo-6-(trifluoromethyl)-1,2-dihydropyridine-3-carboxamide;
5-(4-(((1S,3R,6R)-7,7-difluoro-2,5-dioxabicyclo[4.1.0]heptan-3-yl)methoxy)phenyl)-2-oxo-6-(trifluoromethyl)-1,2-dihydropyridine-3-carboxamide;
aka: 5-(4-((cis-7,7-difluoro-2,5-dioxabicyclo[4.1.0]heptan-3-yl)methoxy)phenyl)-2-oxo-6-(trifluoromethyl)-1,2-dihydropyridine-3-carboxamide;
5-(4-(((1R,3R,6S)-7,7-difluoro-2,5-dioxabicyclo[4.1.0]heptan-3-yl)methoxy)phenyl)-2-oxo-6-(trifluoromethyl)-1,2-dihydropyridine-3-carboxamide;
aka: 5-(4-((trans-7,7-difluoro-2,5-dioxabicyclo[4.1.0]heptan-3-yl)methoxy)phenyl)-2-oxo-6-(trifluoromethyl)-1,2-dihydropyridine-3-carboxamide;
5-(4-((6-(methoxymethyl)-5-methyl-1,4-dioxan-2-yl)methoxy)phenyl)-2-oxo-6-(trifluoromethyl)-1,2-dihydropyridine-3-carboxamide;
5-(4-(((1S,3S,6R)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide;
5-(4-(((1R,3S,6S)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide;
5-(4-(((1R,3R,6S)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide;
5-(4-(((1S,3R,6R)-7,7-difluoro-1-methyl-2,5-dioxabicyclo[4.1.0]heptan-3-yl)methoxy)phenyl)-2-oxo-6-(trifluoromethyl)-1,2-dihydropyridine-3-carboxamide;
5-(4-(((2S,6S)-6-(methoxymethyl)-1,4-dioxan-2-yl)methoxy)phenyl)-2-oxo-6-(trifluoromethyl)-1,2-dihydropyridine-3-carboxamide;
5-(4-(((2S,6R)-6-(methoxymethyl)-1,4-dioxan-2-yl)methoxy)phenyl)-2-oxo-6-(trifluoromethyl)-1,2-dihydropyridine-3-carboxamide;
5-(4-(((2R,6R)-6-(methoxymethyl)-1,4-dioxan-2-yl)methoxy)phenyl)-2-oxo-6-(trifluoromethyl)-1,2-dihydropyridine-3-carboxamide;
5-(4-(((2R,6S)-6-(methoxymethyl)-1,4-dioxan-2-yl)methoxy)phenyl)-2-oxo-6-(trifluoromethyl)-1,2-dihydropyridine-3-carboxamide;
5-(4-(((2S,6R)-6-(isopropoxymethyl)-1,4-dioxan-2-yl)methoxy)phenyl)-2-oxo-6-(trifluoromethyl)-1,2-dihydropyridine-3-carboxamide;
5-(4-(((2S,6S)-6-(isopropoxymethyl)-1,4-dioxan-2-yl)methoxy)phenyl)-2-oxo-6-(trifluoromethyl)-1,2-dihydropyridine-3-carboxamide;
5-(4-(((2S,6S)-6-cyclopropyl-1,4-dioxan-2-yl)methoxy)phenyl)-2-oxo-6-(trifluoromethyl)-1,2-dihydropyridine-3-carboxamide;
5-(4-(((2S,6S)-6-(methoxymethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-(methoxymethyl)-6-methyl-1,4-dioxan-2-yl)methoxy)phenyl)-2-oxo-6-(trifluoromethyl)-1,2-dihydropyridine-3-carboxamide;
5-(4-(((2R,6R)-6-(methoxymethyl)-6-methyl-1,4-dioxan-2-yl)methoxy)phenyl)-2-oxo-6-(trifluoromethyl)-1,2-dihydropyridine-3-carboxamide;
5-(4-(((2R,6S)-6-(methoxymethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-(dimethylcarbamoyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-((methoxy-d3)methyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-(cyanomethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-methyl-6-((methylsulfonyl)methyl)-1,4-dioxan-2-yl)methoxy)phenyl)-2-oxo-6-(trifluoromethyl)-1,2-dihydropyridine-3-carboxamide;
5-(4-(((2S,6R)-6-((cyclopropylsulfonyl)methyl)-6-methyl-1,4-dioxan-2-yl)methoxy)phenyl)-2-oxo-6-(trifluoromethyl-1,2-dihydropyridine-3-carboxamide;
(S)-5-(4-((6,6-bis(methoxymethyl)-1,4-dioxan-2-yl)methoxy)phenyl)-2-oxo-6-(trifluoromethyl)-1,2-dihydropyridine-3-carboxamide;
(S)-5-(4-((2,5,8-trioxaspiro[3.5]nonan-6-yl)methoxy)phenyl)-2-oxo-6-(trifluoromethyl)-1,2-dihydropyridine-3-carboxamide;
(S)-5-(4-((1,4,8,11-tetraoxaspiro[5.6]dodecan-2-yl)methoxy)phenyl)-2-oxo-6-(trifluoromethyl)-1,2-dihydropyridine-3-carboxamide;
5-(4-(((2S,6S)-6-(fluoromethyl)-6-(methoxymethyl)-1,4-dioxan-2-yl)methoxy)phenyl)-2-oxo-6-(trifluoromethyl)-1,2-dihydropyridine-3-carboxamide;
5-(4-(((2S,6S)-6-(chloromethyl)-6-(methoxymethyl)-1,4-dioxan-2-yl)methoxy)phenyl)-2-oxo-6-(trifluoromethyl)-1,2-dihydropyridine-3-carboxamide;
5-(4-(((2S,6S)-6-(fluoromethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-(fluoromethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((3-fluoroazetidin-1-yl)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((3,3-difluoroazetidin-1-yl)methyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-((dimethylamino)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((dimethylamino)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((difluoromethoxy)methyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-(2-methoxyethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-methyl-6-((oxetan-3-yloxy)methyl)-1,4-dioxan-2-yl)methoxy)phenyl)-2-oxo-6-(trifluoromethyl)-1,2-dihydropyridine-3-carboxamide;
5-(4-(((2S,6S)-6-(ethoxymethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-methyl-6-(((3-methyl)oxetan-3-yl)oxy)methyl)-1,4-dioxan-2-yl)methoxy)phenyl)-2-oxo-6-(trifluoromethyl)-1,2-dihydropyridine-3-carboxamide;
5-(4-(((2S,6S)-6-((2-methoxyethoxy)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((2,2-difluoroethoxy)methyl)-6-methyl-1,4-dioxan-2-yl)methoxy)phenyl)-2-oxo-6-(trifluoromethyl-1,2-dihydropyridine-3-carboxamide;
5-(4-(((2S,6R)-6-methyl-6-((2,2,2-trifluoroethoxy)methyl)-1,4-dioxan-2-yl)methoxy)phenyl)-2-oxo-6-(trifluoromethyl)-1,2-dihydropyridine-3-carboxamide;
5-(4-(((2S,6S)-6-(hydroxymethyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-(2,2-difluoroethyl)-6-methyl-1,4-dioxan-2-yl)methoxy)phenyl)-2-oxo-6-(trifluoromethyl)-1,2-dihydropyridine-3-carboxamide;
5-(4-(((2S,6S)-6-(2-fluoroethyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-((2-fluoroethoxy)methyl)-6-methyl-1,4-dioxan-2-yl)methoxy)phenyl)-2-oxo-6-(trifluoromethyl)-1,2-dihydropyridine-3-carboxamide;
5-(4-(((2S,6R)-6-cyclopropyl-6-methyl-1,4-dioxan-2-yl)methoxy)phenyl)-2-oxo-6-(trifluoromethyl)-1,2-dihydropyridine-3-carboxamide;
5-(4-(((2S,6R)-6-cyclopropyl-6-(methoxymethyl)-1,4-dioxan-2-yl)methoxy)phenyl)-2-oxo-6-(trifluoromethyl)-1,2-dihydropyridine-3-carboxamide;
5-(4-(((2S,6S)-6-cyclopropyl-6-(methoxymethyl)-1,4-dioxan-2-yl)methoxy)phenyl)-2-oxo-6-(trifluoromethyl)-1,2-dihydropyridine-3-carboxamide,
and/or
5-(4-(((5R,7S)-2,6,9-trioxaspiro[4.5]decan-7-yl)methoxy)phenyl)-2-oxo-6-(trifluoromethyl)-1,2-dihydropyridine-3-carboxamide.

28. A pharmaceutical composition comprising a therapeutically effective amount of a compound of claim 1, a stereoisomer thereof or a pharmaceutically acceptable salt thereof, and a pharmaceutically acceptable carrier.

29. A method of inhibiting influenza virus nucleoprotein (NP), which comprises contacting NP with a compound of claim 1, a stereoisomer thereof or a pharmaceutically acceptable salt thereof.

30. A method of preventing or treating influenza, which comprises administering to a subject a therapeutically effective amount of a compound of claim 1, a stereoisomer thereof or a pharmaceutically acceptable salt thereof.

31. A pharmaceutical composition comprising a compound of any of claims 1 to 27, a stereoisomer thereof or a pharmaceutically acceptable salt thereof, and one or more pharmaceutically acceptable carriers.

32. A combination comprising a therapeutically effective amount of a compound according to any one of claims 1 to 27, a stereoisomer thereof or a pharmaceutically acceptable salt thereof, and one or more therapeutically active co-agents.

33. A method of treating influenza, comprising administering to a subject in need thereof a therapeutically effective amount of a compound of any of claims 1-27, a stereoisomer thereof or a pharmaceutically acceptable salt thereof.

34. A compound according to any one of claims 1 to 27, a stereoisomer thereof or a pharmaceutically acceptable salt thereof, for use in the treatment of influenza.

35. Use of a compound according to any one of claims 1 to 27, a stereoisomer thereof or a pharmaceutically acceptable salt thereof, in the manufacture of a medicament for the treatment of influenza.
